SEQUENCE LISTING (2 sequences)

SEQ ID NO: 1 - RNA, 23 nt, synthetic construct
  source 1..23: mol_type = other RNA
  modified_base 1: mod_base = OTHER; note = 5'-vinylphosphonate modification
  modified_base 2: mod_base = OTHER; note = 2'-F sugar modification
  modified_base 3: mod_base = um
  modified_base 4: mod_base = cm
  modified_base 5: mod_base = gm
  modified_base 6: mod_base = OTHER; note = 2'-F sugar modification
  modified_base 7: mod_base = OTHER; note = 2'-O-methyl sugar modification
  modified_base 8: mod_base = OTHER; note = 2'-F sugar modification
  modified_base 9: mod_base = OTHER; note = 2'-F sugar modification
  modified_base 10: mod_base = OTHER; note = 2'-O-methyl sugar modification
  modified_base 11: mod_base = OTHER; note = 2'-O-methyl sugar modification
  modified_base 12: mod_base = um
  modified_base 13: mod_base = cm
  modified_base 14: mod_base = OTHER; note = 2'-F sugar modification
  modified_base 15: mod_base = OTHER; note = 2'-O-methyl sugar modification
  modified_base 16: mod_base = OTHER; note = 2'-F sugar modification
  modified_base 17: mod_base = cm
  modified_base 17: mod_base = OTHER; note = 2'-O-methyl sugar modification
  modified_base 19: mod_base = gm
  modified_base 20: mod_base = um
  modified_base 21: mod_base = OTHER; note = 2'-O-methyl sugar modification
  modified_base 22: mod_base = gm
  modified_base 23: mod_base = cm
  modified_base 1..3: mod_base = OTHER; note = Phosphorothioate internucleoside linkage
  modified_base 21..23: mod_base = OTHER; note = Phosphorothioate internucleoside linkage
tttcgaatcaatccaacagtagc

SEQ ID NO: 2 - RNA, 22 nt, synthetic construct
  source 1..22: mol_type = other RNA
  modified_base 1: mod_base = um
  modified_base 2: mod_base = OTHER; note = 2'-O-methyl sugar modification
  modified_base 3: mod_base = cm
  modified_base 4: mod_base = um
  modified_base 5: mod_base = gm
  modified_base 6: mod_base = um
  modified_base 7: mod_base = OTHER; note = 2'-F sugar modification
  modified_base 8: mod_base = gm
  modified_base 9: mod_base = OTHER; note = 2'-F sugar modification
  modified_base 10: mod_base = OTHER; note = 2'-F sugar modification
  modified_base 11: mod_base = OTHER; note = 2'-F sugar modification
  modified_base 12: mod_base = um
  modified_base 13: mod_base = gm
  modified_base 14: mod_base = OTHER; note = 2'-O-methyl sugar modification
  modified_base 15: mod_base = um
  modified_base 16: mod_base = um
  modified_base 17: mod_base = cm
  modified_base 18: mod_base = gm
  modified_base 19: mod_base = OTHER; note = 2'-O-methyl sugar modification
  modified_base 20: mod_base = OTHER; note = 2'-O-methyl sugar modification
  modified_base 21: mod_base = OTHER; note = 2'-O-methyl sugar modification
  modified_base 22: mod_base = OTHER; note = Inverted abasic deoxyribose
  modified_base 1..3: mod_base = OTHER; note = Phosphorothioate internucleoside linkage
  modified_base 21..22: mod_base = OTHER; note = Phosphorothioate internucleoside linkage
tactgttggattgattcgaaan

(19) United States
(12) Patent Application Publication
(10) Pub. No.: US 2026/0000775 A1
(43) Pub. Date: Jan. 1, 2026

(54) a4ß1/7 INTEGRIN LIGAND CONJUGATED COMPOUNDS AND USES THEREOF

(71) Applicant: ADARx Pharmaceuticals, Inc., San Diego, CA (US)

(72) Inventors: Zhen Li, San Diego, CA (US); Rui Zhu, San Diego, CA (US); Mehdi Michel Djamel Numa, San Diego, CA (US); Mihai Azimioara, San Diego, CA (US)

(73) Assignee: ADARx Pharmaceuticals, Inc., San Diego, CA (US)

(21) Appl. No.: 18/852,103

(22) PCT Filed: Apr. 4, 2023
(86) PCT No.: PCT/US2023/017482

§ 371 Date: Sep. 27, 2024

Related U.S. Application Data
(60) Provisional application No. 63/327,334, filed on Apr. 4, 2022.

(51) Int. Cl. A61K 47/64; C07K 14/705

(52) U.S. Cl. CPC A61K 47/6425; C07K 14/7055

(57) ABSTRACT

Provided herein are α₄β_{1/7} integrin receptor ligand-containing compounds, methods of delivering said compounds, and methods of treating diseases, disorders, and symptoms (e.g., central nervous system diseases, disorders, and symptoms) in a subject using said compounds.

### CROSS-REFERENCE TO RELATED APPLICATIONS

This application is a National Stage filing under 35 U.S.C. 371 of International PCT Application Serial No. PCT/US2023/017482, filed Apr. 4, 2023, which claims the benefit of priority under 35 U.S.C. § 119(e) of U.S. Provisional Application No. 63/327,334, filed Apr. 4, 2022. The disclosure of the prior application is considered part of and is incorporated by reference in its entirety in the disclosure of this application.

### REFERENCE TO AN ELECTRONIC SEQUENCE LISTING

The contents of the electronic sequence listing (A127870012US01-SEQ-TNG.xml; Size: 25,819 bytes; and Date of Creation: Apr. 24, 2025) are herein incorporated by reference in its entirety.

### BACKGROUND

In the use of compounds in therapeutic, prophylactic, or diagnostic applications, it is often desirable that the compounds be delivered to a specific location (for example, to desired cell(s)) to enhance the therapeutic or prophylactic effect or to be advantageous for diagnostic purposes. This is frequently the case when attempting to deliver a therapeutic compound in vivo. Further, being able to efficiently deliver a compound to a specific location can limit or potentially eliminate unintended consequences (such as off-target effects) that may be caused by administration of the compound. One strategy to facilitate delivery of a compound, such as a therapeutic, prophylactic, or diagnostic compound, to a desired location in vivo, is by linking or attaching the compound to a targeting ligand.

One class of compounds that can be targeted using targeting ligands are oligomeric compounds such as, for example, proteins, peptides, antibodies, and oligonucleotides. Oligomeric compounds that include nucleotide sequences (e.g., oligonucleotides) at least partially complementary to a target nucleic acid have been shown to alter the function and activity of the target both in vitro and in vivo. When delivered to a cell containing a target nucleic acid (such as mRNA or pre-mRNA), oligonucleotides have been shown to modulate the expression or activity of the target nucleic acid. In certain instances, the oligonucleotide can reduce the expression of the gene by inhibiting translation of the nucleic acid target and/or triggering the degradation of the target nucleic acid.

If the target nucleic acid is mRNA, one mechanism by which an oligonucleotide can modulate the expression of the mRNA target is through RNA interference. RNA interference is a biological process by which RNA or RNA-like molecules (such as chemically modified RNA molecules) are able to silence gene expression, at least in part, through the RNA-induced silencing Complex (RISC) pathway. Additionally, oligonucleotides can modulate the expression of a target nucleic acid, such as a target mRNA, through an RNase recruitment mechanism, microRNA mechanisms, occupancy-based mechanisms, and editing mechanisms.

Oligonucleotides may be single-stranded or double-stranded. Oligonucleotides may comprise DNA, RNA, and RNA-like molecules, which can also include modified nucleosides including one or more modified sugars, modified nucleobases, and modified internucleoside linkages.

Another class of compounds that can be targeted using targeting ligands are small molecule compounds. The small molecule compounds (e.g., an organic compound having a molecular weight of ca. 1000 daltons or less) are typically shown to alter the function and/or activity of the target such that disease and/or disease symptoms are modulated or ameliorated or are typically useful as a diagnostic marker when localized to the target. More efficient delivery of a compound to a specific location can limit or potentially eliminate unintended consequences (such as off-target effects) that may be caused by administration of the compound and provide improved localization of a diagnostic compound.

### SUMMARY

Embodiments provided herein are directed to compounds (e.g., any of those delineated herein) and methods for targeting cells expressing α₄β_{1/7} integrin receptor and/or α₄β_{1/7} integrin receptor (referred to herein collectively as “α₄β₁₇ integrin receptor”). Certain embodiments provided herein are directed to compounds and methods for delivering an agent to cells expressing α₄β₁₇ integrin receptor. In certain embodiments, the cell is in the brain. In certain embodiments, the cell is in the frontal cortex. In certain embodiments, the cell is in the striatum. In certain embodiments, the cell is in the cerebellum. In certain embodiments, the cell is in the brain stem. In certain embodiments, the cell is in the hippocampus. In certain embodiments, the cell is in the spinal cord. In certain embodiments, the agent is a therapeutic compound. In certain embodiments, delivery of the agent is for the treatment of diseases, disorders, and symptoms in a subject. In certain embodiments, the agent is a diagnostic compound. In certain embodiments, a compound comprises an α₄β_{1/7} integrin receptor ligand and one or more linker moieties for attachment to a therapeutic, prophylactic, or diagnostic agent. In certain embodiments, a compound comprises an α₄β_{1/7} integrin receptor ligand, one or more linker moieties, and a therapeutic agent. In certain embodiments, the therapeutic agent is selected from a small molecule or an oligomeric compound. In certain embodiments, the oligomeric compound is a protein, a peptide, an antibody, an oligonucleotide, or a combination thereof. In certain embodiments, the α₄β_{1/7} integrin receptor ligand is an α₄β_{1/7} integrin receptor agonist. In certain embodiments, the α₄β_{1/7} integrin receptor ligand is an α₄β_{1/7} integrin receptor antagonist. In certain embodiments, the α₄β_{1/7} integrin receptor ligand is a small molecule, an aptamer, a peptide, or an antibody. In certain embodiments, the α₄β_{1/7} integrin receptor ligand is any of those delineated herein, or a derivative or prodrug thereof.

In certain embodiments, contacting a cell expressing α₄β_{1/7} integrin receptor, such as a brain cell, with a compound provided herein, delivers the agent to the cell. In certain embodiments, contacting a cell expressing α₄β_{1/7} integrin receptor, such as a brain cell, with a compound provided herein, treats a disease, disorder, or symptom in a subject. In certain embodiments, a compound comprising an α₄β_{1/7} integrin receptor ligand selectively or preferentially targets a cell expressing α₄β_{1/7} integrin receptor compared to a cell not expressing α₄β_{1/7} integrin receptor. In certain embodiments, a compound comprising an α₄β_{1/7} integrin receptor ligand selectively or preferentially targets a cell expressing α₄β_{1/7} integrin receptor compared to a compound not comprising an α₄β_{1/7} integrin receptor ligand.

Certain embodiments provided herein are directed to compounds and methods for modulating expression of a nucleic acid target in cells expressing an α₄β_{1/7} integrin receptor. In certain embodiments, the cell is in the brain. In certain embodiments, the cell is in the frontal cortex. In certain embodiments, the cell is in the striatum. In certain embodiments, the cell is in the cerebellum. In certain embodiments, the cell is in the brain stem. In certain embodiments, the cell is in the hippocampus. In certain embodiments, the cell is in the spinal cord. In certain embodiments, contacting a cell expressing an α₄β_{1/7} integrin receptor, such as a brain cell, with a compound provided herein, modulates the expression or activity of a nucleic acid target in the cell. In certain embodiments, a compound comprises an α₄β_{1/7} integrin receptor ligand, one or more linker moieties, and an oligonucleotide.

It is understood that the embodiments provided herein with respect to preferred variable selections can be taken alone or in combination with one or more embodiments, or other preferred variable selections provided herein, as if each combination were explicitly listed herein.

In one aspect, the present disclosure provides compounds, and stereoisomers, tautomers, prodrugs, and salts thereof, comprising the structure of Formula (I′):

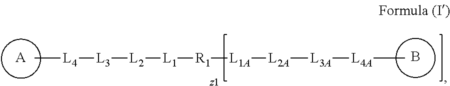

wherein:
each of

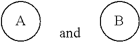

and is independently an α₄ in integrin ligand;
each of L₁, L₂, L₃, L₄, L_{1A}, L_{2A}, L_{3A}, and L_{4A} is independently a linker, a bond, or absent;
R¹ comprises one or more oligonucleotides, protecting groups, small molecules, proteins, antibodies, and/or peptides; and
z1 is 0 or 1.

In some embodiments, the compound, or a stereoisomer, tautomer, prodrug, or salt thereof, comprises the structure of Formula (I”):

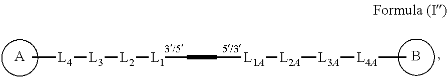

wherein:
[symbol] is an oligonucleotide, and wherein L₁, L₂, L₃, L₄, L_{1A}, L_{2A}, L_{3A}, and L_{4A} are as defined herein.

In some embodiments, the compound, or a stereoisomer, tautomer, prodrug, or salt thereof, comprises the structure of Formula (I):

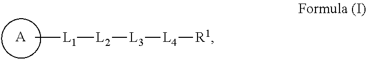

wherein R¹, L₁, L₂, L₃, and L₄ are as defined herein.

In some embodiments, the α₄β_{1/7} integrin ligand is an α₄β_{1/7} integrin agonist. In some embodiments, the α₄β_{1/7} integrin ligand is an α₄β_{1/7} integrin antagonist. In certain embodiments, the α₄β_{1/7} integrin ligand is selected from the group consisting of:

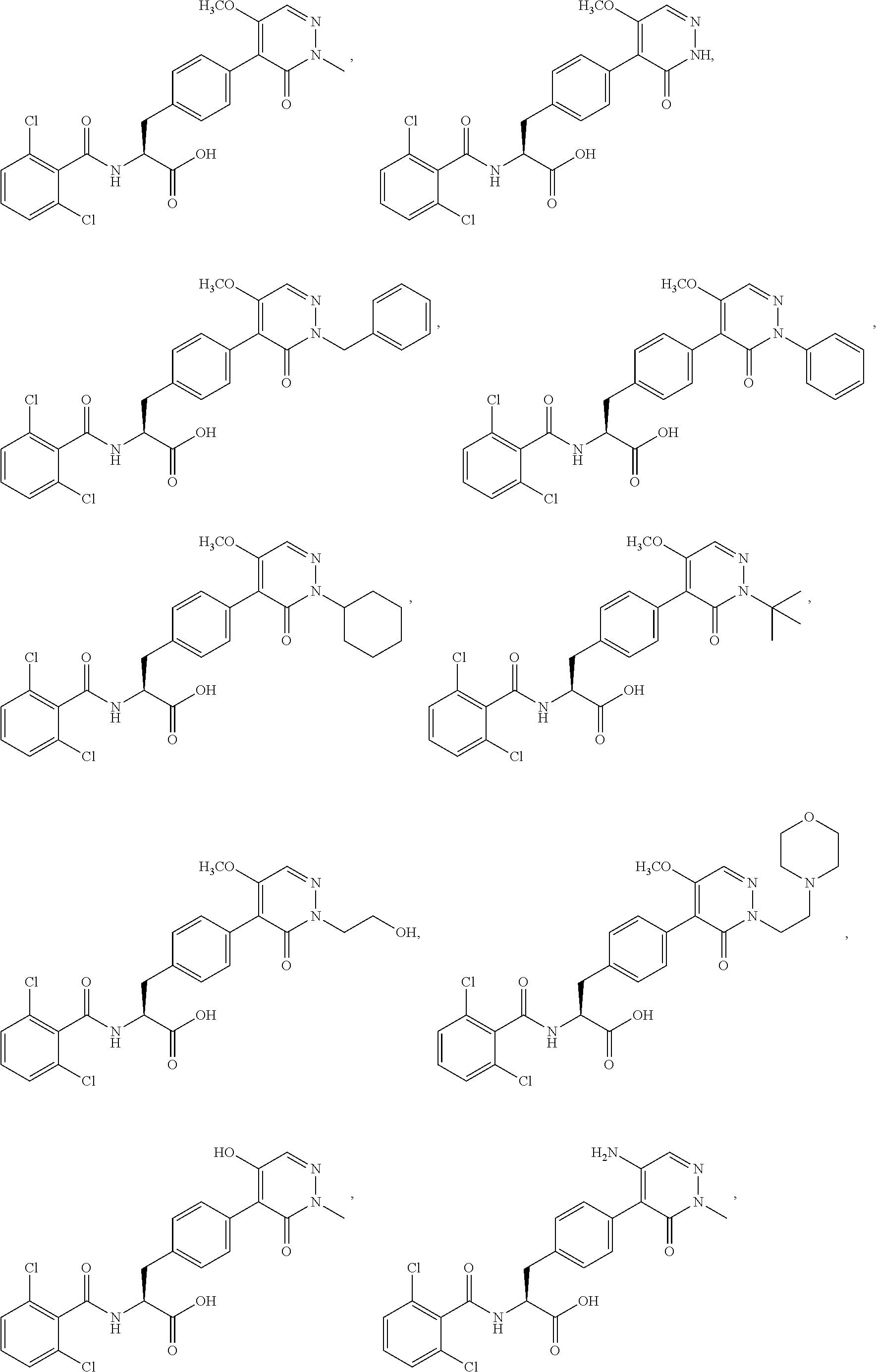

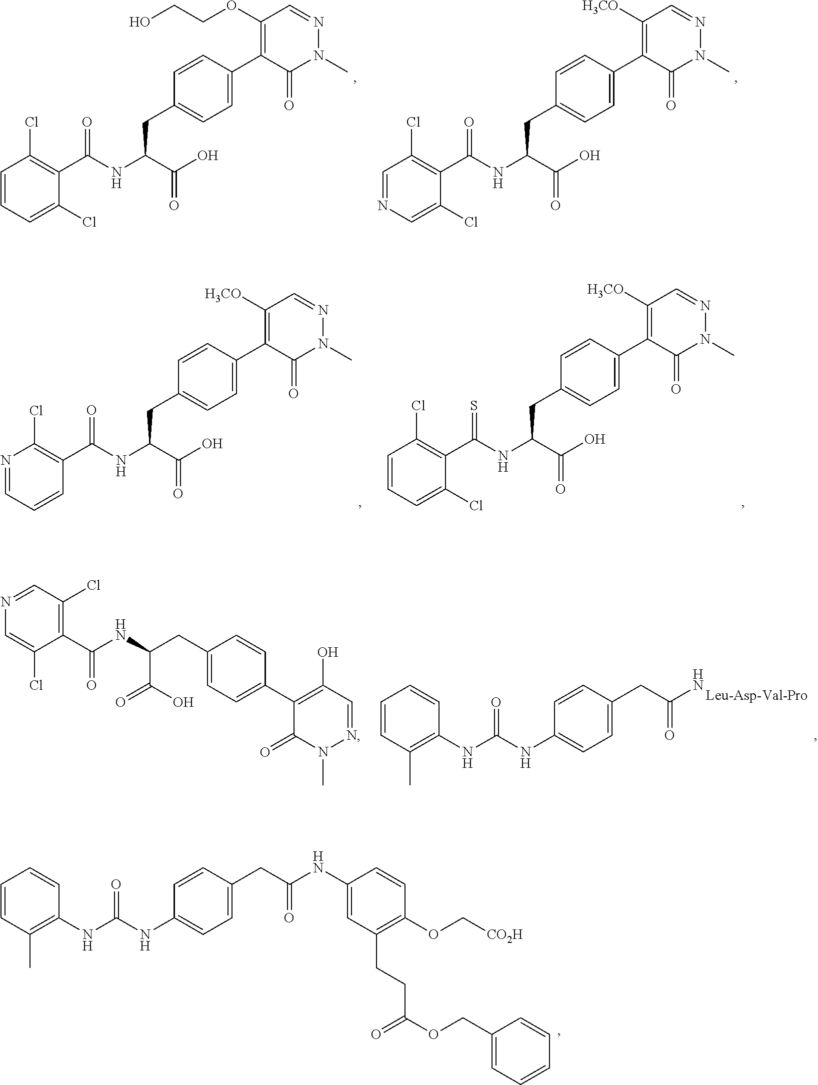

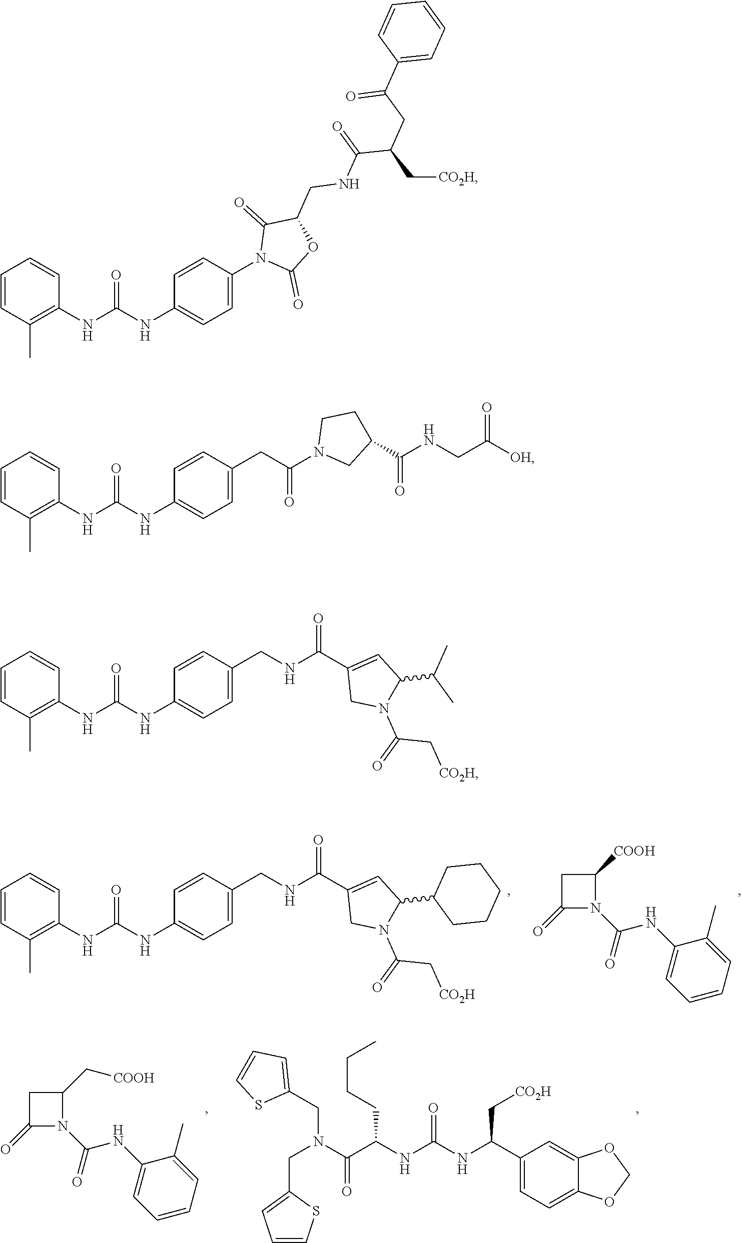

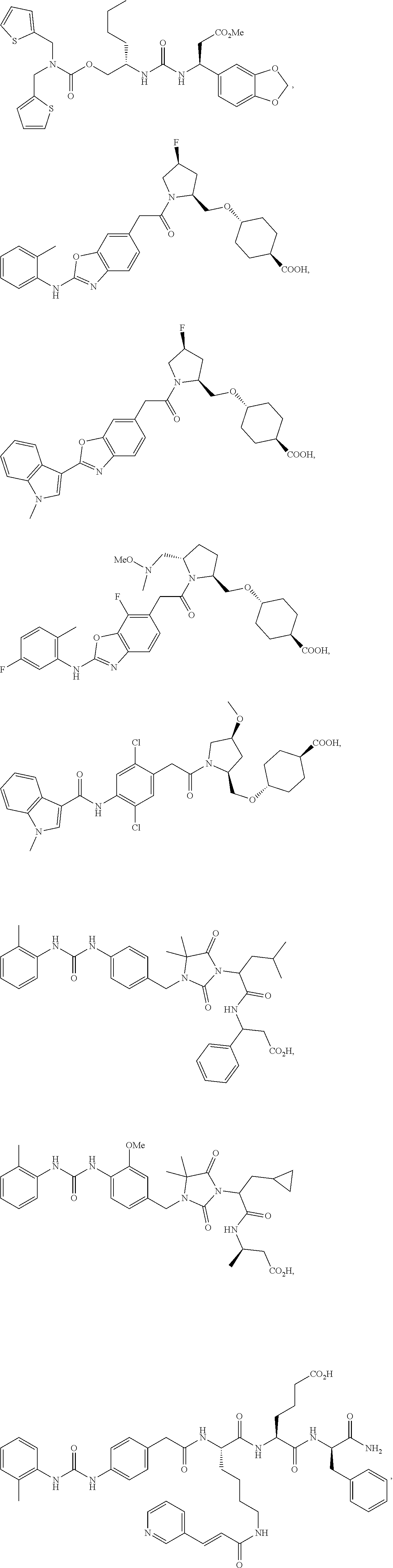

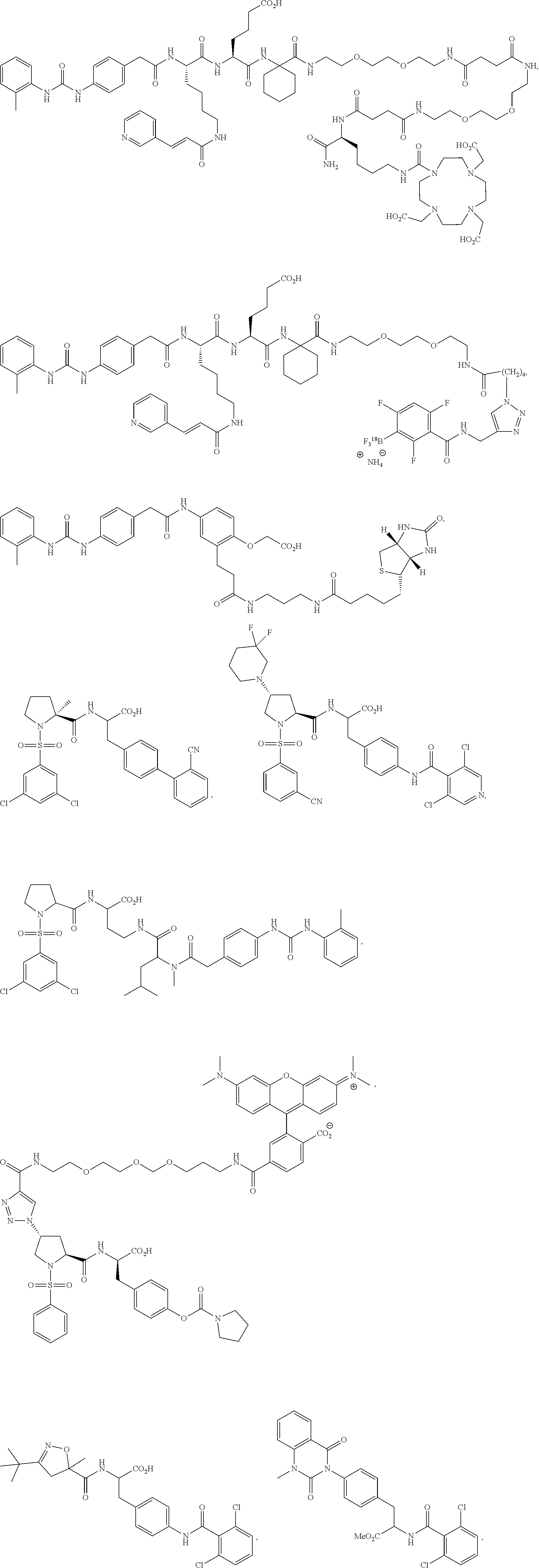

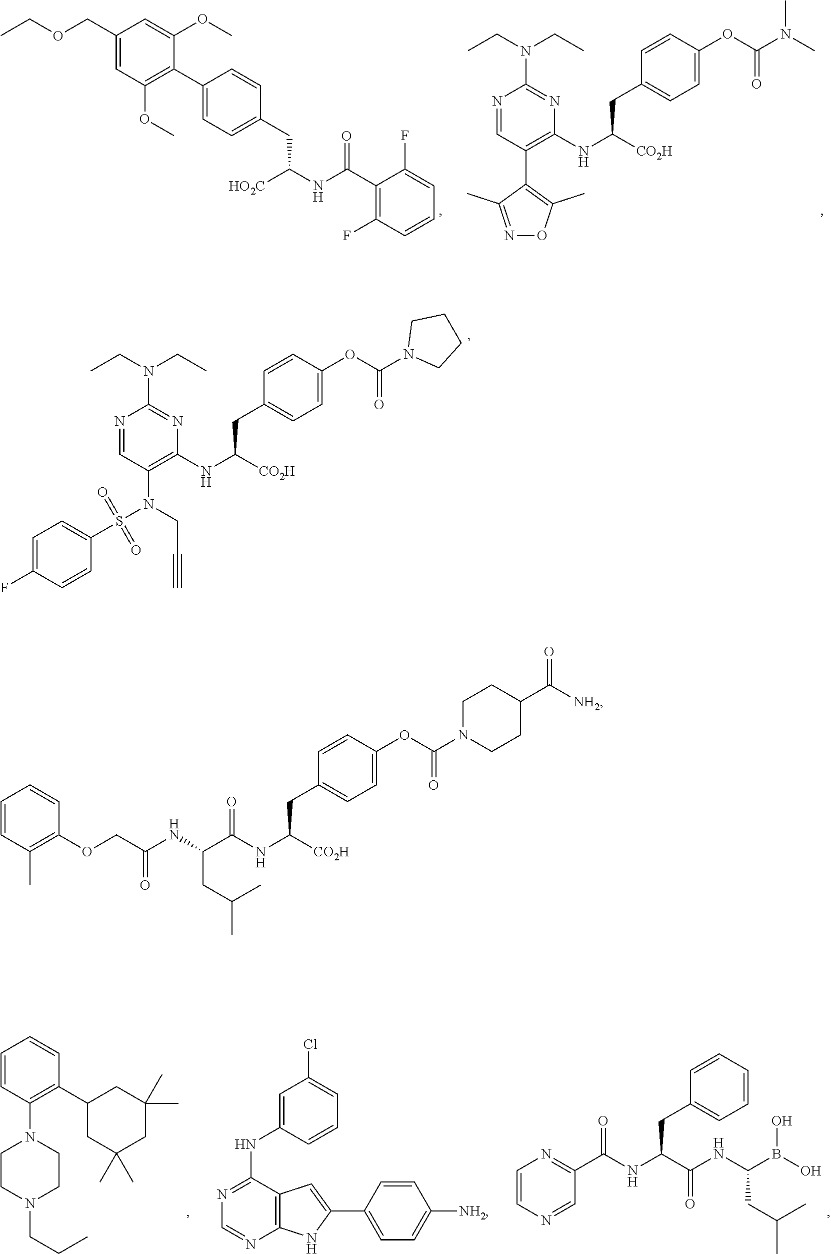

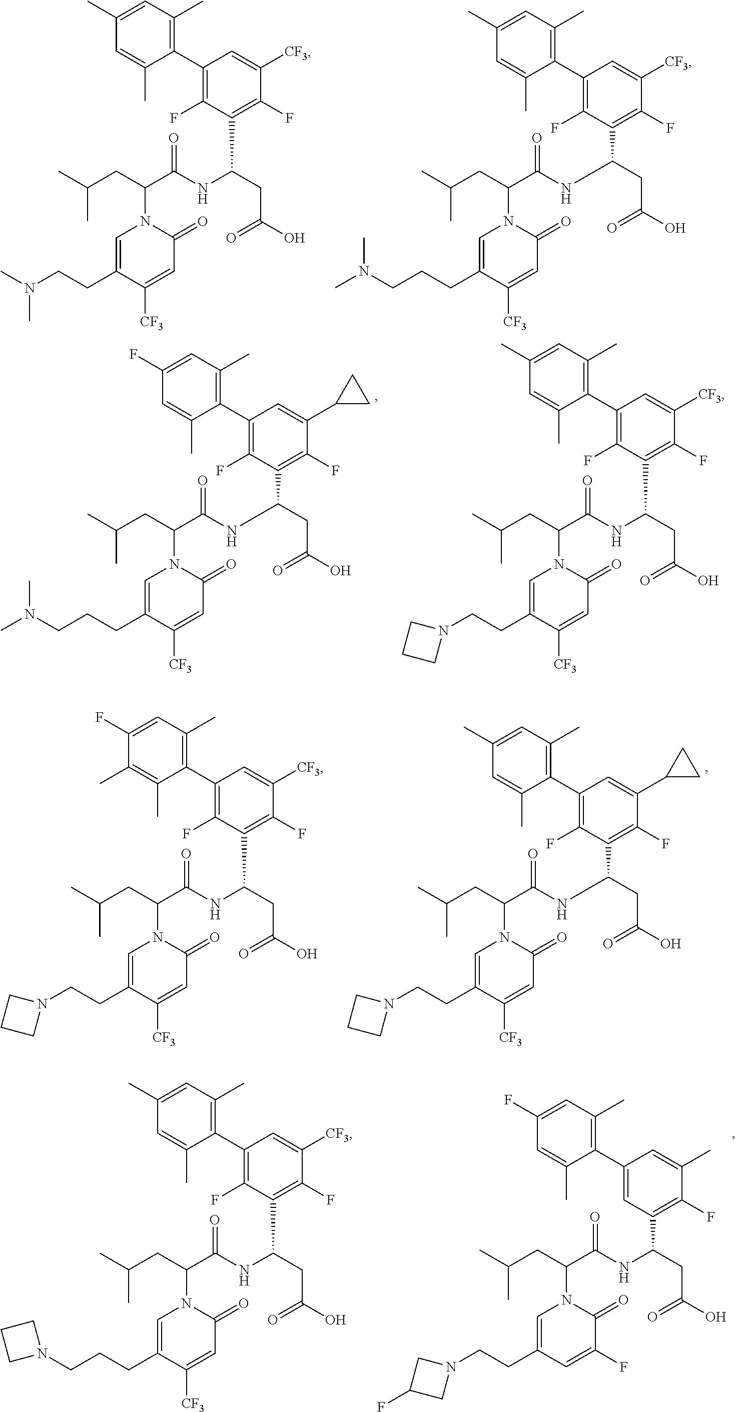

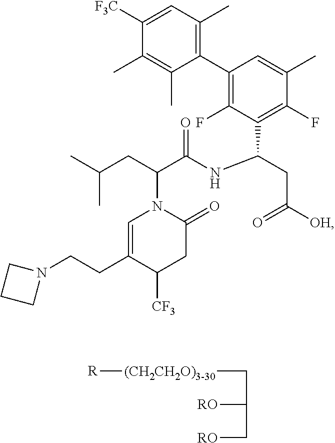

wherein each instance of R is independently

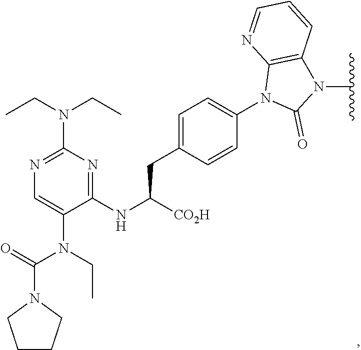 [SMILES: *n1c(=O)n(-c2ccc(C[C@H](Nc3nc(N(CC)CC)ncc3N(CC)C(=O)N3CCCC3)C(=O)O)cc2)c2ncccc21]

an anti-α₄β_{1/7} integrin antibody, and derivatives thereof.

In some embodiments, the compound, or a stereoisomer, tautomer, prodrug, or salt thereof, wherein the compound comprises the structure of Formula (II′):

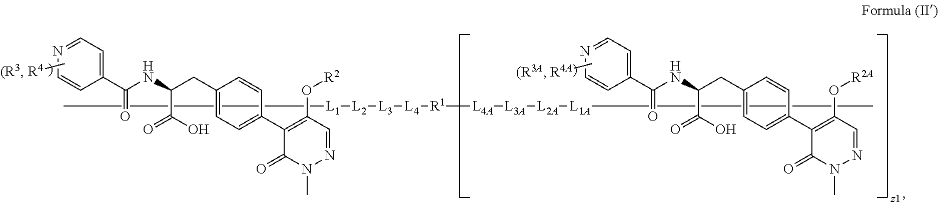

wherein
R² and R^{2A} are each independently H, polyethylene glycol (PEG), optionally substituted heteroalkyl, or optionally substituted heteroaryl;
R³, R^{3A}, R⁴, and R^{4A} are each independently H, halogen, optionally substituted alkyl, or optionally substituted —O-alkyl; and
R¹, z1, L₁, L₂, L₃, L₄, L_{1A}, L_{2A}, L_{3A}, and L_{4A} are as defined herein..

In some embodiments, the compound, or a stereoisomer, tautomer, prodrug, or salt thereof, comprises the structure of Formula (II″):

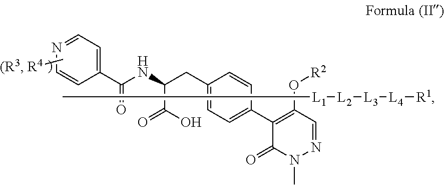

wherein L₁, L₂, L₃, L₄, R¹, R², R³, and R⁴ are as defined herein.

In some embodiments, the compound, or a stereoisomer, tautomer, prodrug, or salt thereof, comprises the structure of Formula (II″-a):

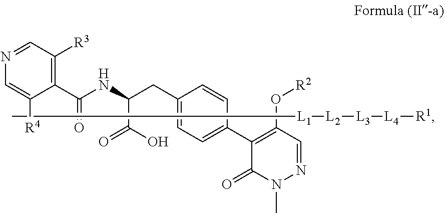

wherein L₁, L₂, L₃, L₄, R¹, R², R³, and R⁴ are as defined herein.

In some embodiments, the compound, or a stereoisomer, tautomer, prodrug, or salt thereof, wherein the compound comprises the structure of Formula (II″-a-1):

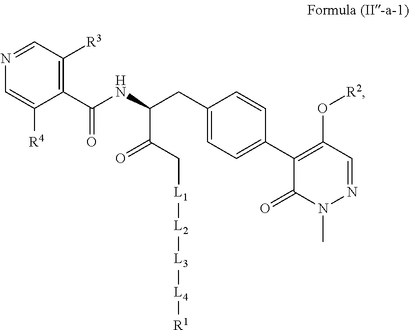

wherein L₁, L₂, L₃, L₄, R¹, R², R³, and R⁴ are as defined herein.

In some embodiments, the compound, or a stereoisomer, tautomer, prodrug, or salt thereof, comprises the structure of Formula (II″-a-2):

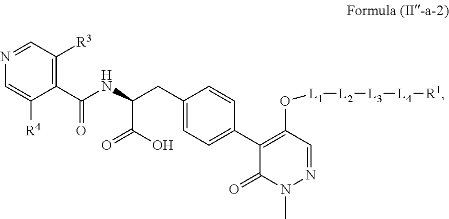

wherein L₁, L₂, L₃, L₄, R¹, R³, and R⁴ are as defined herein.

In some embodiments, the α₄β_{1/7} integrin ligand comprises the structure

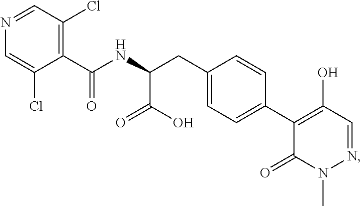 [SMILES: Cn1ncc(O)c(-c2ccc(C[C@H](NC(=O)c3c(Cl)cncc3Cl)C(=O)O)cc2)c1=O]

or a derivative thereof.

In some embodiments, the compound, or a stereoisomer, tautomer, prodrug, or salt thereof, comprises the structure of Formula (II):

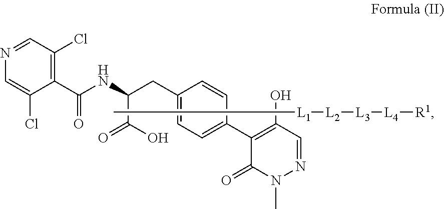

wherein R¹, L₁, L₂, L₃, and L₄ are as defined herein.

In some embodiments, the compound, or a stereoisomer, tautomer, prodrug, or salt thereof, comprises the structure of Formula (II-a):

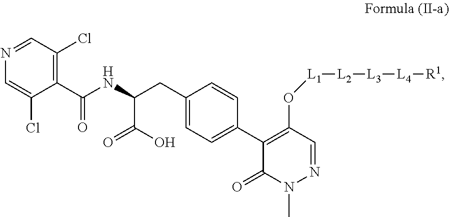

wherein R¹, L₁, L₂, L₃, and L₄ are as defined herein.

In some embodiments, the compound, or a stereoisomer, tautomer, prodrug, or salt thereof, comprises the structure of Formula (III′):

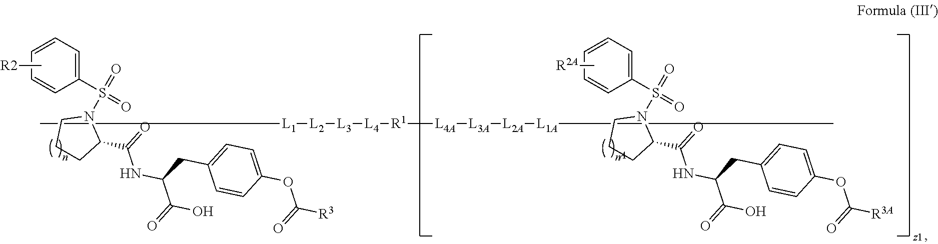

wherein
R² and R^{2A} are each independently H, halogen, polyethylene glycol (PEG), optionally substituted alkyl, optionally substituted heteroalkyl, optionally substituted heteroaryl, optionally substituted —O-alkyl, or optionally substituted cycloalkyl;
R³ and R^{3A} are each independently optionally substituted heteroalkyl or optionally substituted heterocyclyl;
n and nA are each independently 1, 2, or 3;
z1 is 0 or 1; and
R¹, L₁, L₂, L₃, L₄, L_{1A}, L_{2A}, L_{3A}, and L_{4A} are as defined herein.

In some embodiments, the compound, or a stereoisomer, tautomer, prodrug, or salt thereof, comprises the structure of Formula (III):

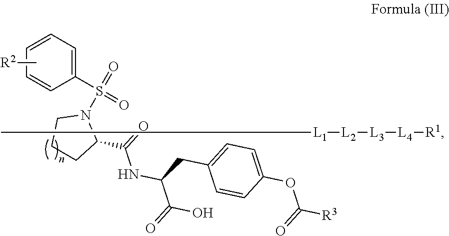

wherein n, R¹, R², R³, L₁, L₂, L₃, and L₄ are as defined herein.

In some embodiments, the compound, or a stereoisomer, tautomer, prodrug, or salt thereof, comprises the structure of Formula (III-a):

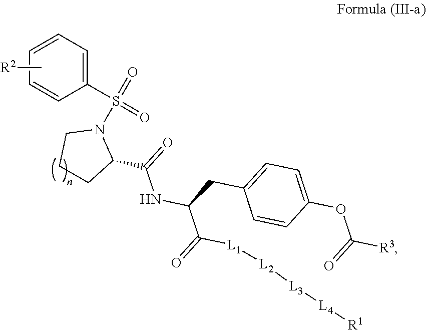

wherein n, R¹, R², R³, L₁, L₂, L₃, and L₄ are as defined herein.

In some embodiments, the compound, or a stereoisomer, tautomer, prodrug, or salt thereof, comprises the structure of Formula (III-b):

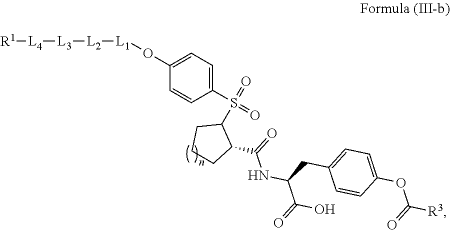

wherein n, R¹, R³, L₁, L₂, L₃, and L₄ are as defined herein.

In some embodiments, the compound, or a stereoisomer, tautomer, prodrug, or salt thereof, comprises the structure of Formula (IV′):

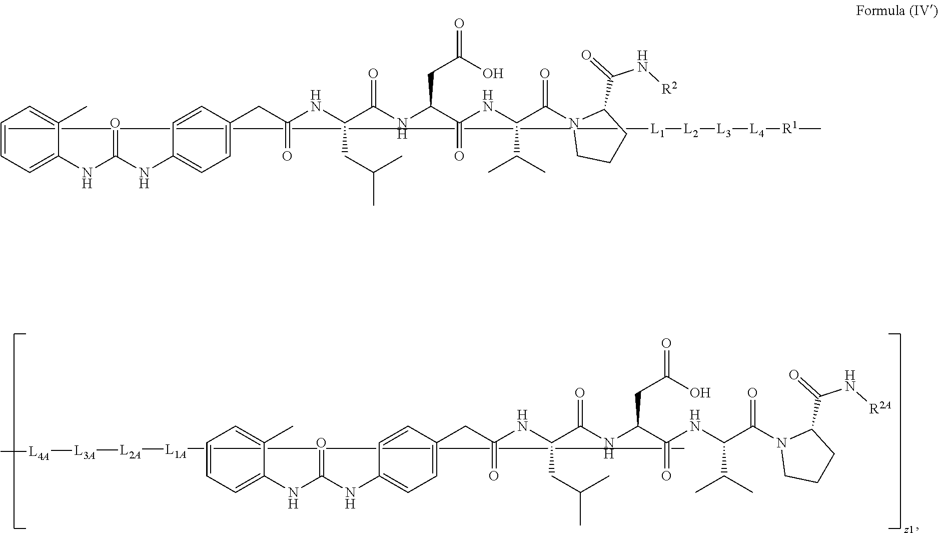

wherein
R² and R^{2A} are each independently H, —OH, —NH₂, —NHR³, —OR³, or absent;
each instance of R³ is independently H, polyethylene glycol, optionally substituted alkyl, optionally substituted heteroalkyl, optionally substituted cycloalkyl, or optionally substituted heteroaryl; and
z1, R¹, L₁, L₂, L₃, L₄, L_{1A}, L_{2A}, L_{3A}, and L_{4A} are as defined herein.

In some embodiments, the compound, or a stereoisomer, tautomer, prodrug, or salt thereof, comprises the structure of Formula (IV):

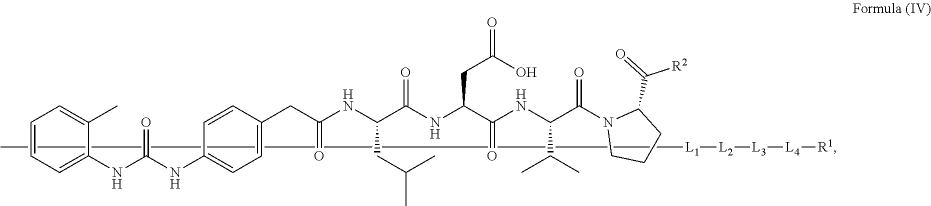

wherein R¹, R², L₁, L₂, L₃, and L₄ are as defined herein.

In some embodiments, the compound, or a stereoisomer, tautomer, prodrug, or salt thereof, comprises the structure of Formula (IV-a):

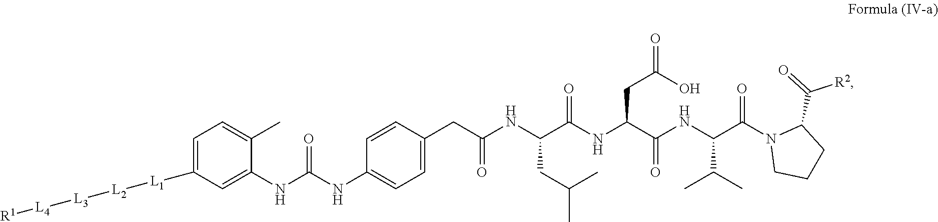

wherein R¹, R², L₁, L₂, L₃, and L₄ are as defined herein.

In some embodiments, the compound, or a stereoisomer, tautomer, prodrug, or salt thereof, comprises the structure of Formula (IV-b):

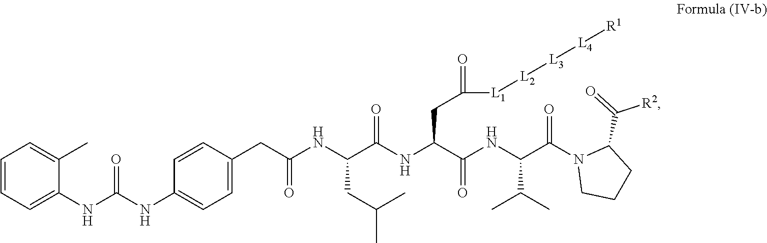

wherein R¹, R², L₁, L₂, L₃, and L₄ are as defined herein.

In some embodiments, the compound, or a stereoisomer, tautomer, prodrug, or salt thereof, comprises the structure of Formula (IV-c):

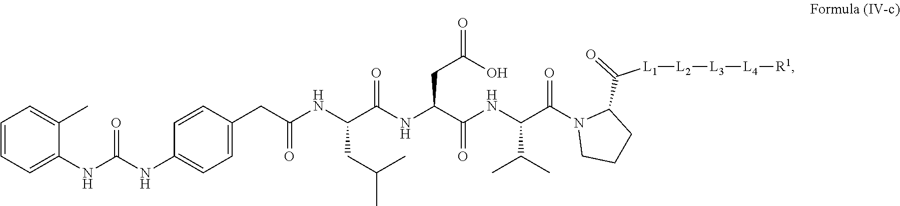

wherein R¹, L₁, L₂, L₃, and L₄ are as defined herein.

In some embodiments, the compound, or a stereoisomer, tautomer, prodrug, or salt thereof, comprises the structure of Formula (V′):

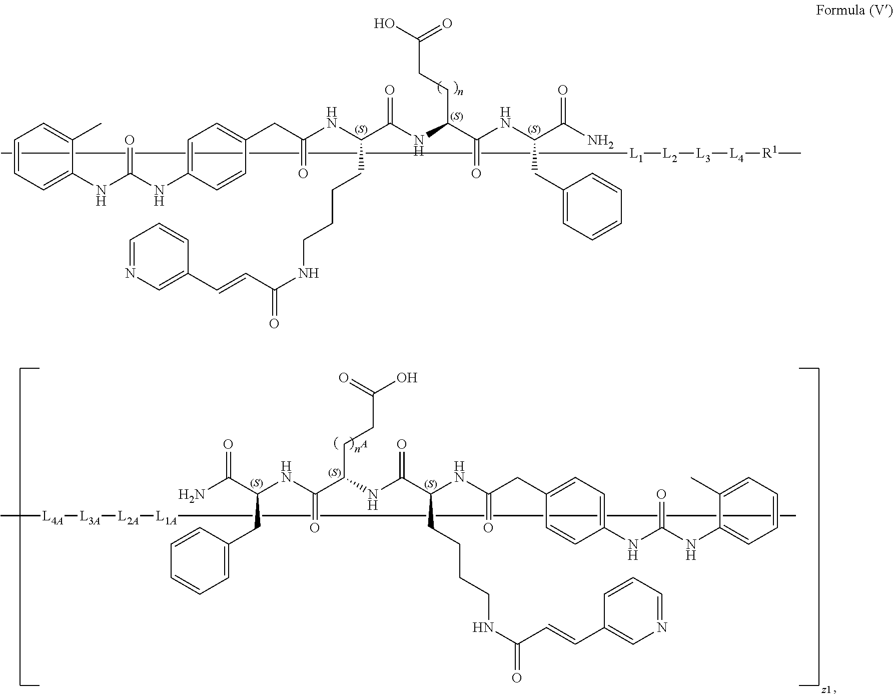

wherein
n and nA are each independently 0, 1, 2, or 3; and
z¹, R¹, L₁, L₂, L₃, L₄, L_{1A}, L_{2A}, L_{3A}, and L_{4A} are as defined herein.

In some embodiments, the compound, or a stereoisomer, tautomer, prodrug, or salt thereof, comprises the structure of Formula (V′-a):

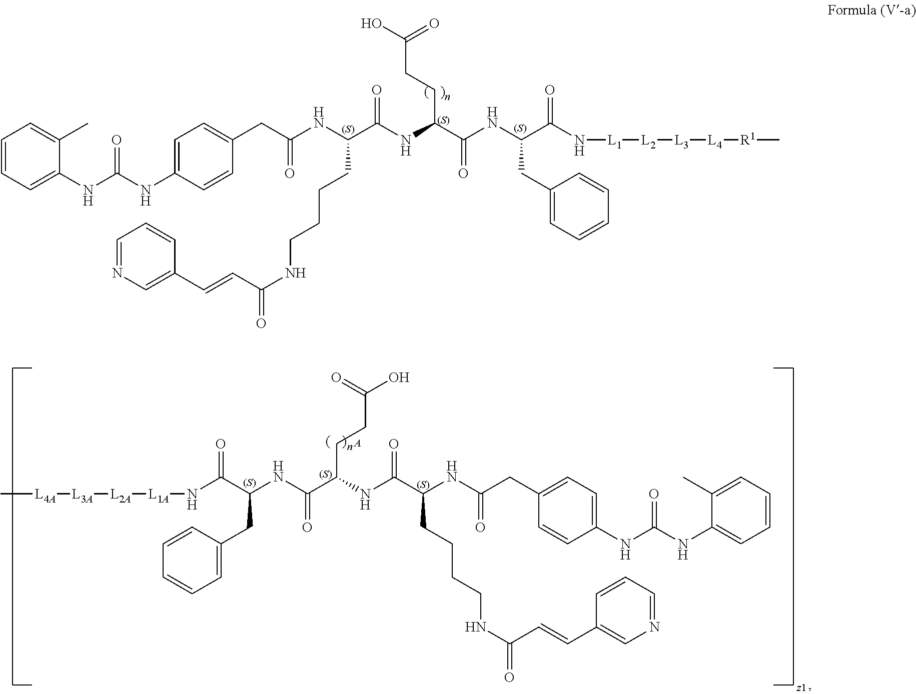

wherein R¹, n, z¹, L₁, L₂, L₃, L₄, L_{1A}, L_{2A}, L_{3A}, and L_{4A} are as defined herein.

In some embodiments, the compound, or a stereoisomer, tautomer, prodrug, or salt thereof, comprises the structure of Formula (V):

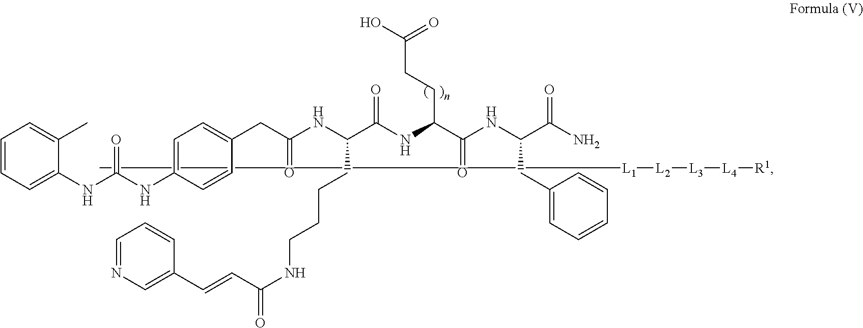

wherein R¹, n, L₁, L₂, L₃, and L₄ are as defined herein.

In some embodiments, the compound, or a stereoisomer, tautomer, prodrug, or salt thereof, comprises the structure of Formula (V-a):

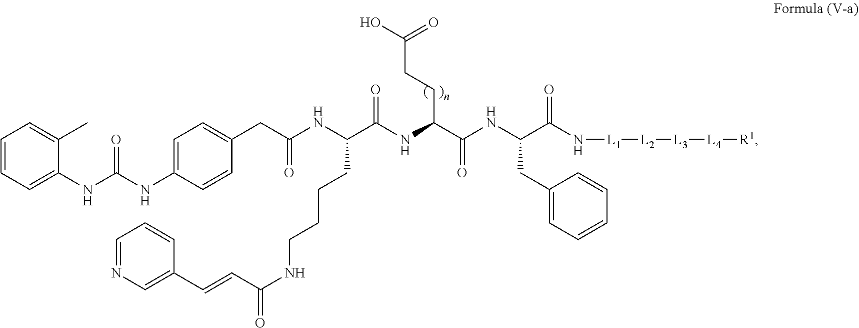

wherein R¹, n, L₁, L₂, L₃, and L₄ are as defined herein.

In some embodiments, the compound, or a stereoisomer, tautomer, prodrug, or salt thereof, comprises the structure of Formula (V-b):

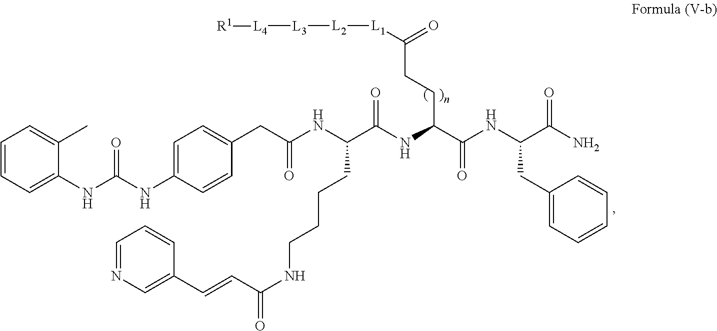

wherein R¹, n, L₁, L₂, L₃, and L₄ are as defined herein.

In some embodiments, the compound, or a stereoisomer, tautomer, prodrug, or salt thereof, comprises the structure of Formula (V-c):

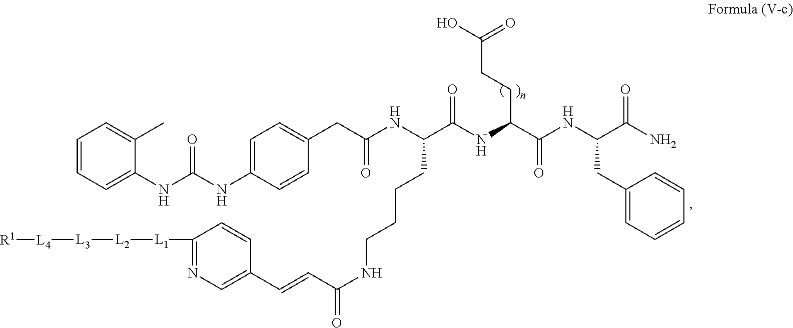

wherein R¹, n, L₁, L₂, L₃, and L₄ are as defined herein.

In some embodiments, the compound, or a stereoisomer, tautomer, prodrug, or salt thereof, comprises the structure of Formula (V-d):

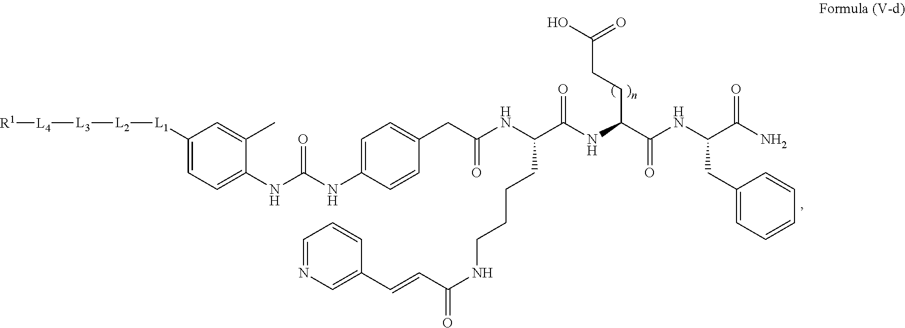

wherein R¹, n, L₁, L₂, L₃, and L₄ are as defined herein.

In some embodiments, the compound, or a stereoisomer, tautomer, prodrug, or salt thereof, comprises the structure of Formula (V-e):

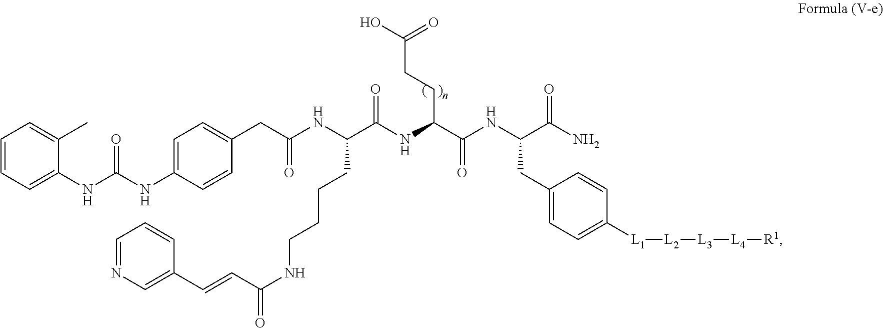

wherein R¹, n, L₁, L₂, L₃, and L₄ are as defined herein.

In some embodiments, the compound, or a stereoisomer, tautomer, prodrug, or salt thereof, wherein the compound comprises the structure of Formula (VI′):

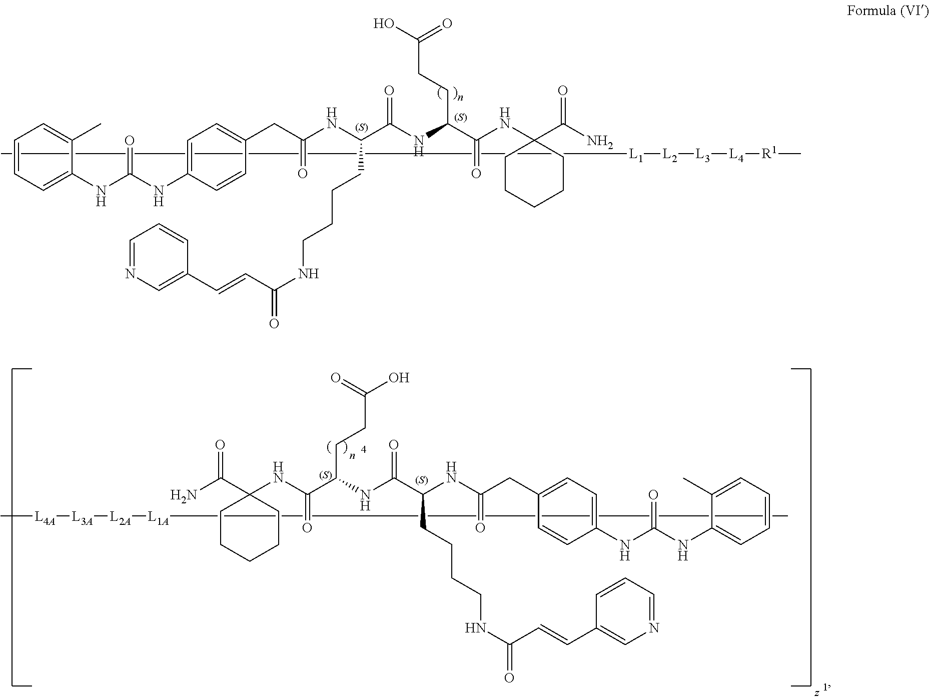

wherein
n and nA are each independently 0, 1, 2, or 3; and
z1, R¹, L₁, L₂, L₃, L₄, L_{1A}, L_{2A}, L_{3A}, and L_{4A} are as defined herein.

In some embodiments, the compound, or a stereoisomer, tautomer, prodrug, or salt thereof, comprises the structure of Formula (VI′-a):

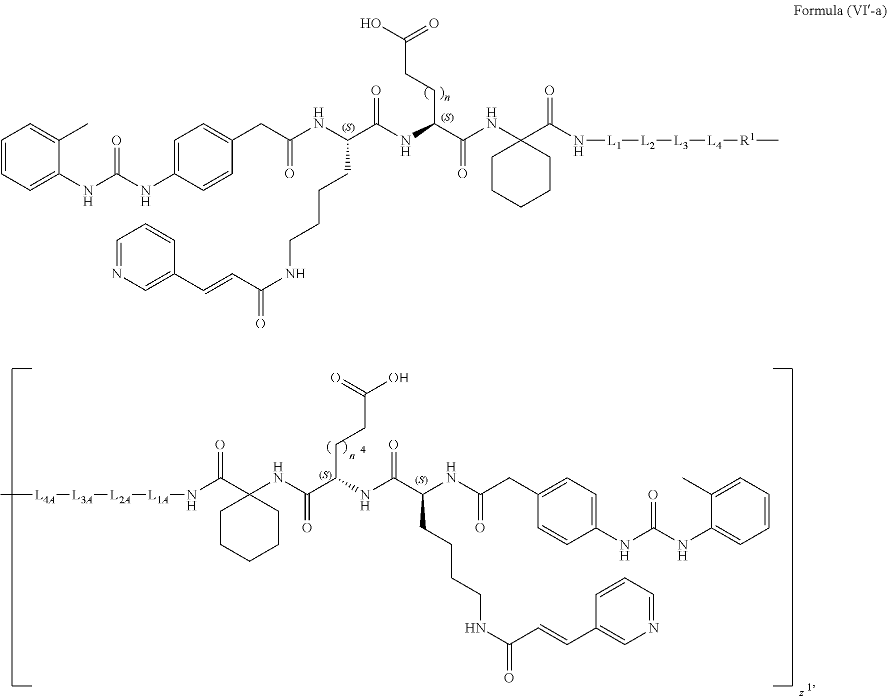

wherein R¹, n, n^{A}, z1, L₁, L₂, L₃, L₄, L_{1A}, L₂A, L₃A, and L_{4A} are as defined herein.

In some embodiments, the compound, or a stereoisomer, tautomer, prodrug, or salt thereof, comprises the structure of Formula (VI):

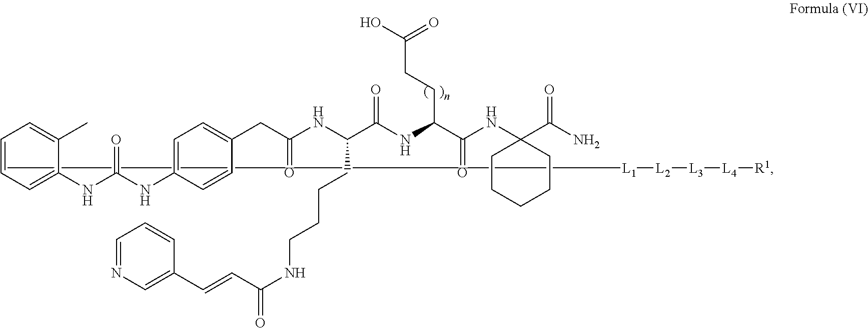

wherein R¹, n, L₁, L₂, L₃, and L₄ are as defined herein.

In some embodiments, the compound, or a stereoisomer, tautomer, prodrug, or salt thereof, comprises the structure of Formula (VI-a):

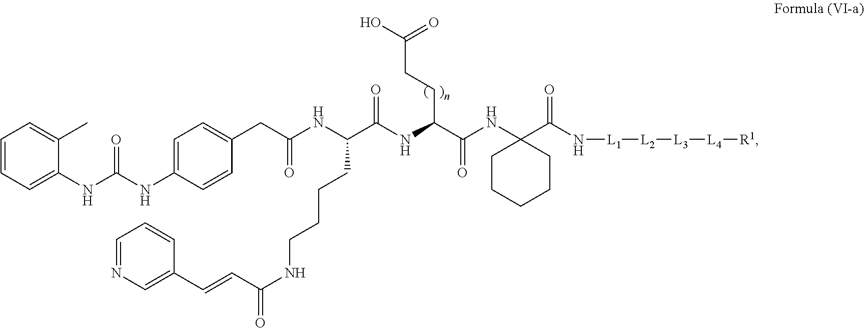

wherein R¹, n, L₁, L₂, L₃, and L₄ are as defined herein.

In some embodiments, the compound, or a stereoisomer, tautomer, prodrug, or salt thereof, comprises the structure of Formula (VI-b):

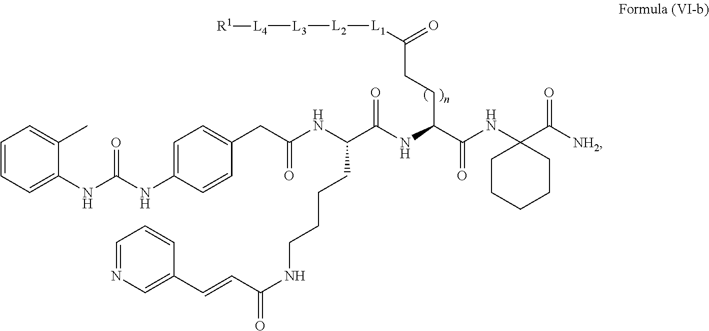

wherein R¹, n, L₁, L₂, L₃, and L₄ are as defined herein.

In some embodiments, the compound, or a stereoisomer, tautomer, prodrug, or salt thereof, comprises the structure of Formula (VI-c):

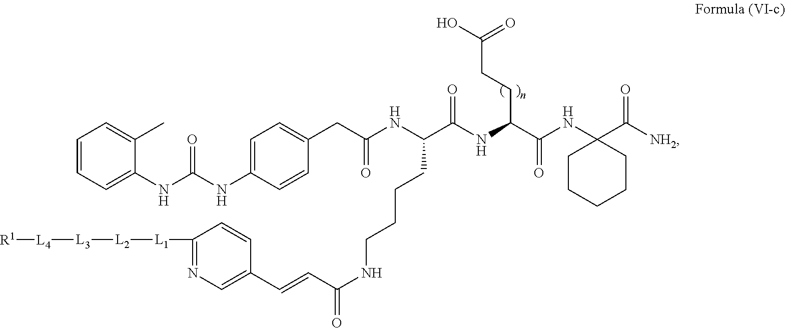

wherein R¹, n, L₁, L₂, L₃, and L₄ are as defined herein.

In some embodiments, the compound, or a stereoisomer, tautomer, prodrug, or salt thereof, comprises the structure of Formula (VI-d):

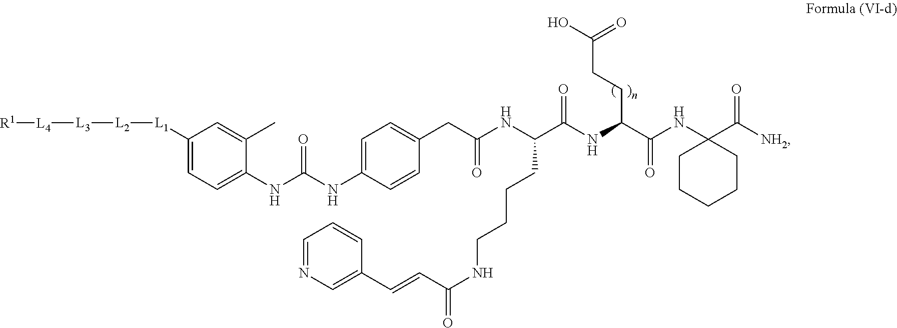

wherein R¹, n, L₁, L₂, L₃, and L₄ are as defined herein.

In some embodiments, the compound, or a stereoisomer, tautomer, prodrug, or salt thereof, comprises the structure of Formula (VII′):

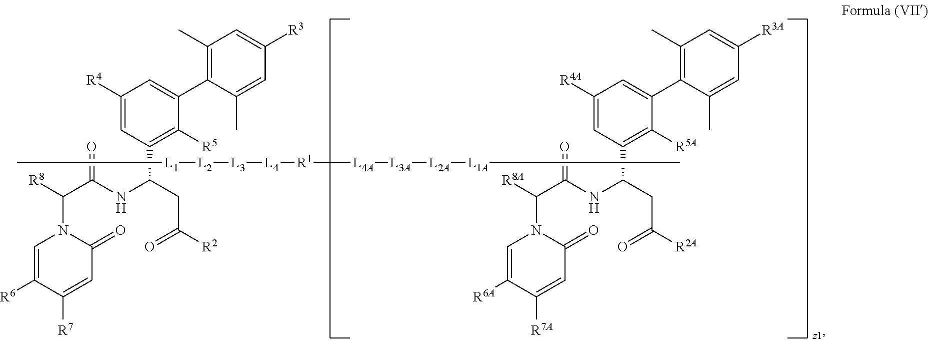

wherein
R², R^{2A}, R³, R^{3A}, R⁴ R^{4A} R⁵, and R^{5A} are each independently H, halogen, optionally substituted alkyl, optionally substituted —O-alkyl, cycloalkyl, or absent;
R⁸ and R^{8A} are each independently optionally substituted C₁-C₅ alkyl, optionally substituted C₁-C₅ alkylene-(C₃-C₆)-cycloalkyl, or optionally substituted (C₁-C₄)-alkylene-(C₁-C₄)-alkoxy;
R⁶, R^{6A}, R⁷, and R^{7A} are each independently H, halogen, alkyl, optionally substituted alkyl, optionally substituted heteroalkyl,

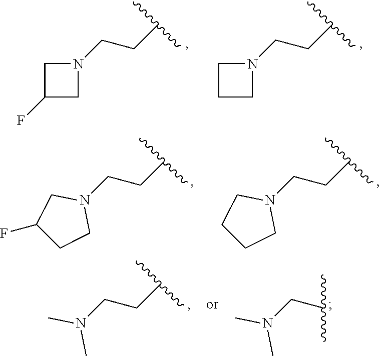

and
R¹, z¹, L₁, L₂, L₃, L₄, L_{4A}, L_{2A}, L_{3A}, and L_{4A} are as defined herein.

In some embodiments, the compound, or a stereoisomer, tautomer, prodrug, or salt thereof, comprises the structure of Formula (VII′-a):

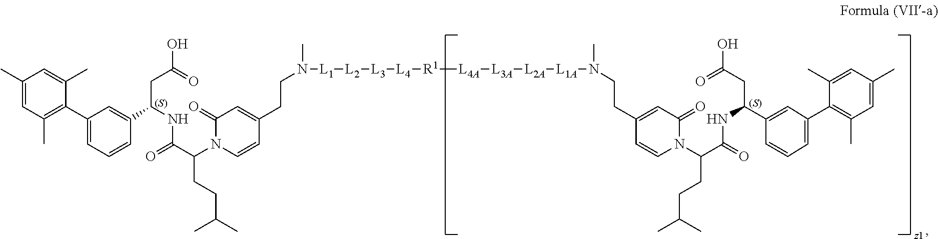

wherein R¹, z¹, L₁, L₂, L₃, L₄, L_{4A}, L_{2A}, L_{3A}, and L_{4A} are as defined herein.

In some embodiments, the compound, or a stereoisomer, tautomer, prodrug, or salt thereof, comprises the structure of Formula (VII′-a-1):

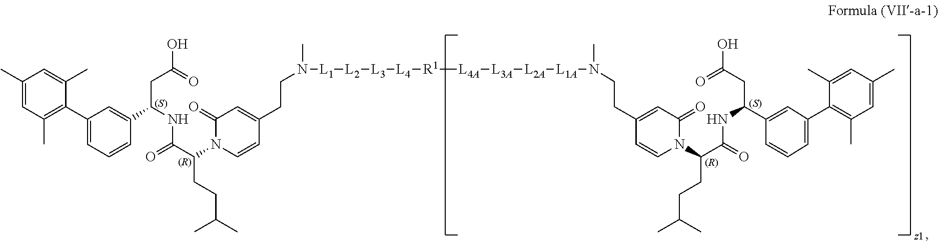

wherein R¹, z¹, L₁, L₂, L₃, L₄, L_{4A}, L_{2A}, L_{3A}, and L_{4A} are as defined herein.

In some embodiments, the compound, or a stereoisomer, tautomer, prodrug, or salt thereof, comprises the structure of Formula (VII′-a-2):

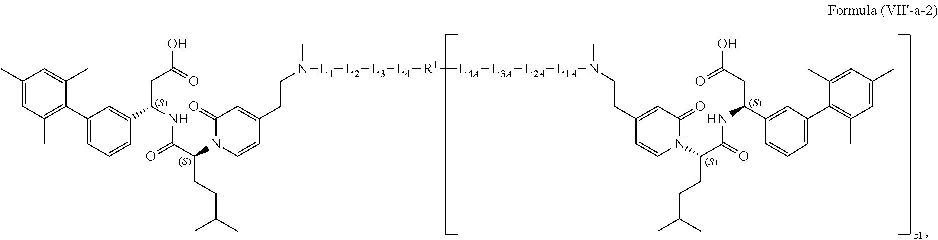

wherein R¹, z¹, L₁, L₂, L₃, L₄, L_{1A}, L_{2A}, L_{3A}, and L_{4A} are as defined herein.

In some embodiments, the compound, or a stereoisomer, tautomer, prodrug, or salt thereof, comprises the structure of Formula (VII):

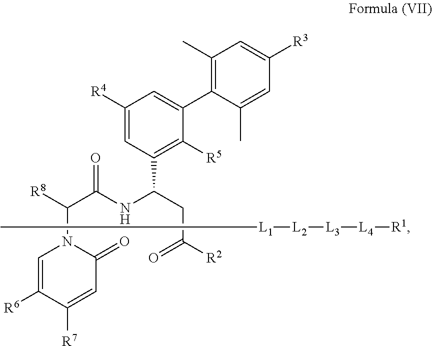

wherein R¹, R², R³, R⁴, R⁵, R⁶, R⁷, R⁸, L₁, L₂, L₃, and L₄ are as defined herein.

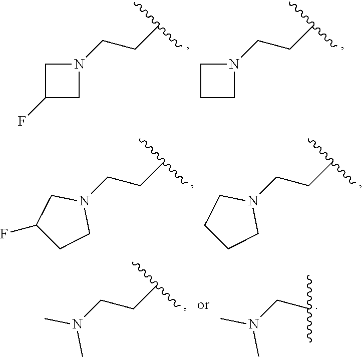

In certain embodiments, R⁶ is F, CF₃, or CH₃, and R⁷ is

In certain embodiments, R⁷ is F,

CF₃, or CH₃, and R⁶ is

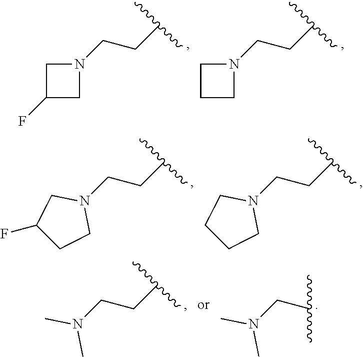

In some embodiments, the compound, or a stereoisomer, tautomer, prodrug, or salt thereof, comprises the structure of Formula (VII-a):

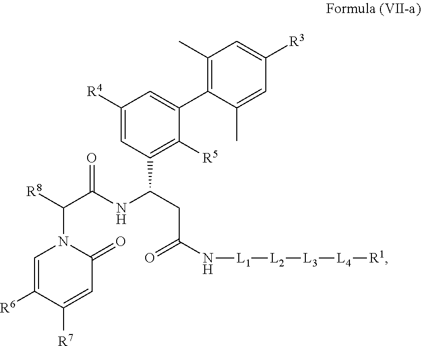

wherein R¹, R³, R⁴,R⁵, R⁶, R⁷, R⁸, L₁, L₂, L₃, and L₄ are as defined herein.

In some embodiments, the compound, or a stereoisomer, tautomer, prodrug, or salt thereof, comprises the structure of Formula (VII-b):

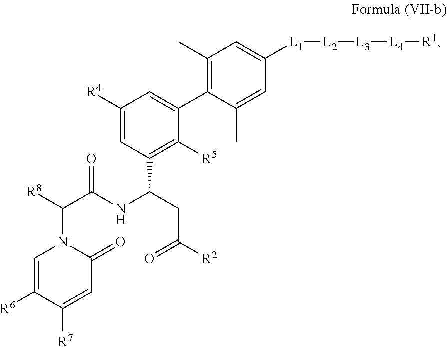

wherein R¹, R², R⁴, R⁵, R⁶, R⁷, R⁸, L₁, L₂, L₃, and L₄ are as defined herein.

In some embodiments, the compound, or a stereoisomer, tautomer, prodrug, or salt thereof, comprises the structure of Formula (VII-c):

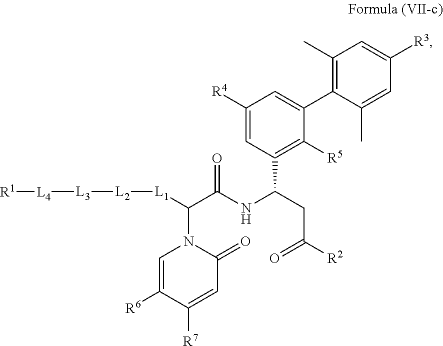

wherein R¹, R², R³, R⁴, R⁵, R⁶, R⁷, L₁, L₂, L₃, and L₄ are as defined herein.

In some embodiments, the compound, or a stereoisomer, tautomer, prodrug, or salt thereof, comprises the structure of Formula (VII-c-1):

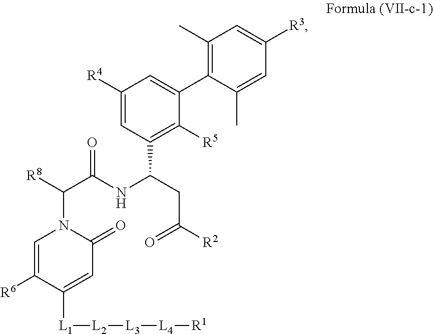

wherein R¹, R², R³, R⁴, R⁵, R⁶, R⁸, L₁, L₂, L₃, and L₄ are as defined herein.

In some embodiments, the compound, or a stereoisomer, tautomer, prodrug, or salt thereof, comprises the structure of Formula (VII-c-2):

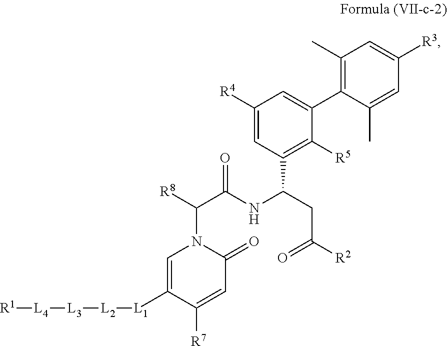

wherein R¹, R², R³, R⁴, R⁵, R⁷, R⁸, L₁, L₂, L₃, and L₄ are as defined herein.

In some embodiments, the compound, or a stereoisomer, tautomer, prodrug, or salt thereof, comprises the structure of Formula (VII-d):

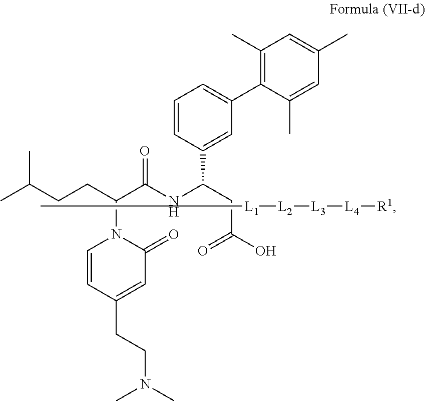

wherein R¹, L₁, L₂, L₃, and L₄ are as defined herein.

In some embodiments, the compound, or a stereoisomer, tautomer, prodrug, or salt thereof, comprises the structure of Formula (VII-d-1):

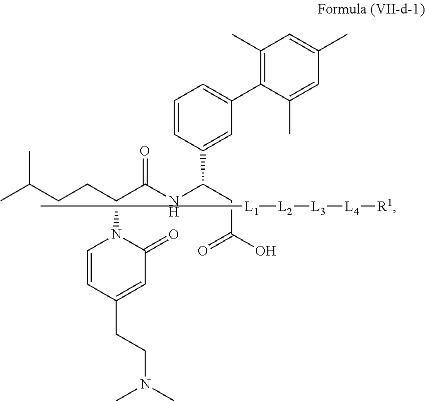

wherein R¹, L₁, L₂, L₃, and L₄ are as defined herein.

In some embodiments, the compound, or a stereoisomer, tautomer, prodrug, or salt thereof, comprises the structure of Formula (VII-d-2):

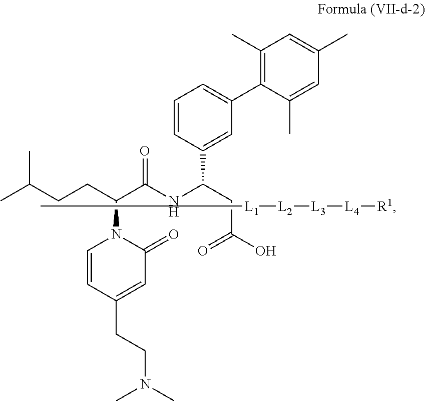

wherein R¹, L₁, L₂, L₃, and L₄ are as defined herein.

In some embodiments, the compound, or a stereoisomer, tautomer, prodrug, or salt thereof, comprises the structure of Formula (VII-d-3):

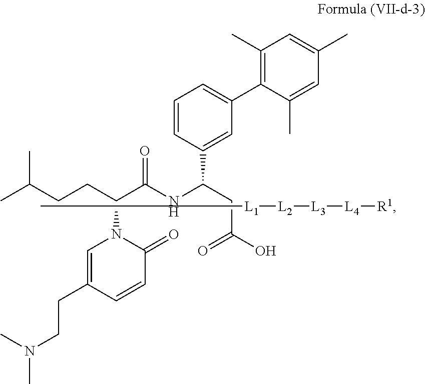

wherein R¹, L₁, L₂, L₃, and L₄ are as defined herein.

In some embodiments, the compound, or a stereoisomer, tautomer, prodrug, or salt thereof, comprises the structure of Formula (VII-d-4):

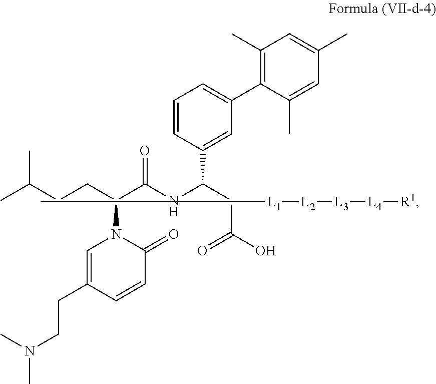

wherein R¹, L₁, L₂, L₃, and L₄ are as defined herein.

In some embodiments, the compound, or a stereoisomer, tautomer, prodrug, or salt thereof, comprises the structure of Formula (VII-d-5):

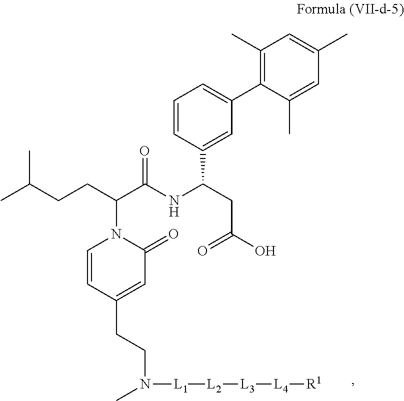

wherein R¹, L₁, L₂, L₃, and L₄ are as defined herein.

In some embodiments, the compound, or a stereoisomer, tautomer, prodrug, or salt thereof, comprises the structure of Formula (VII-d-6):

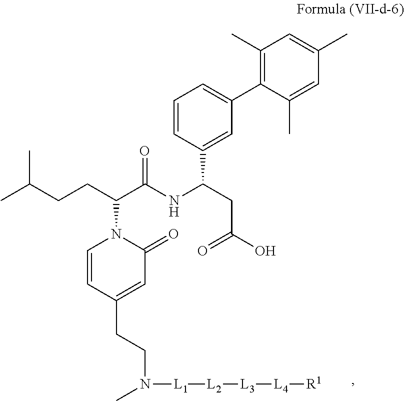

wherein R¹, L₁, L₂, L₃, and L₄ are as defined herein.

In some embodiments, the compound, or a stereoisomer, tautomer, prodrug, or salt thereof, comprises the structure of Formula (VII-d-7):

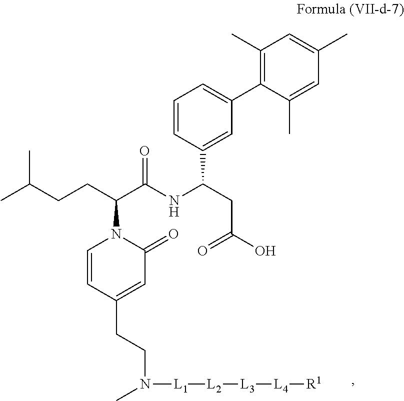

wherein R¹, L₁, L₂, L₃, and L₄ are as defined herein.

In some embodiments, the compound, or a stereoisomer, tautomer, prodrug, or salt thereof, comprises the structure of Formula (VII-d-8):

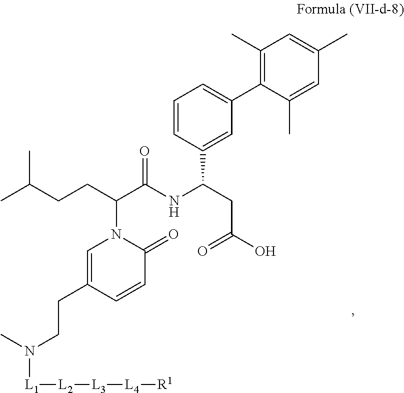

wherein R¹, L₁, L₂, L₃, and L₄ are as defined herein.

In some embodiments, the compound, or a stereoisomer, tautomer, prodrug, or salt thereof, comprises the structure of Formula (VII-d-9):

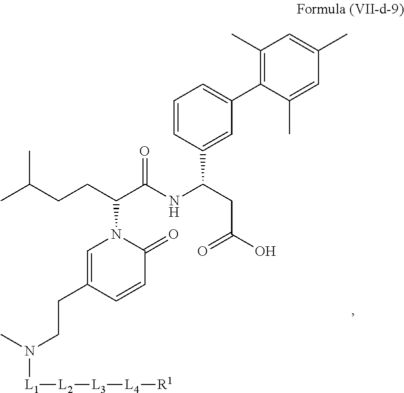

wherein R¹, L₁, L₂, L₃, and L₄ are as defined herein.

In some embodiments, the compound, or a stereoisomer, tautomer, prodrug, or salt thereof, comprises the structure of Formula (VII-d-10):

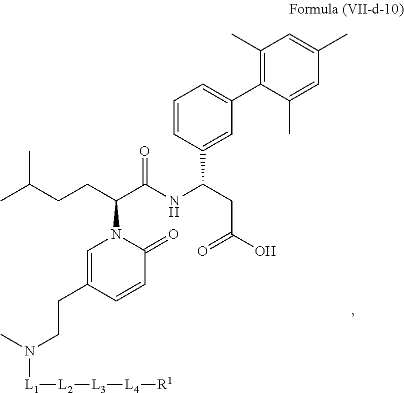

wherein R¹, L₁, L₂, L₃, and L₄ are as defined herein.

In some embodiments, the compound, or a stereoisomer, tautomer, prodrug, or salt thereof, comprises the structure of Formula (VIII′):

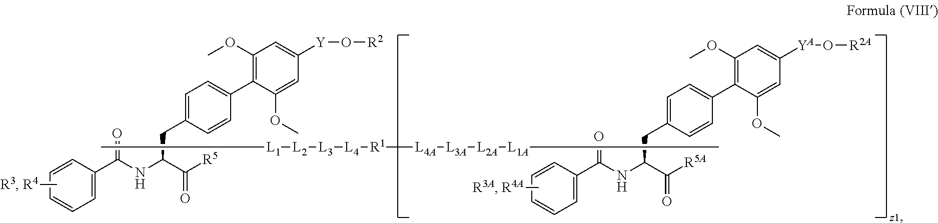

wherein
R² and R^{2A} are each independently H, polyethylene glycol, optionally substituted alkyl, optionally substituted heteroalkyl, optionally substituted cycloalkyl, optionally substituted heteroaryl, or absent;
R³, R^{3A}, R⁴, and R^{4A}, are each independently H, halogen, optionally substituted alkyl, or optionally substituted —O-alkyl;
R⁵ and R^{5A} are each independently —OH or absent;
Y and Y^{A} are each independently —CH₂— or —(CH₂)₂—; and
R¹, z1, L₁, L₂, L₃, L₄, L_{1A}, L_{2A}, L_{3A}, and L_{4A} are as defined herein.

In some embodiments, the compound, or a stereoisomer, tautomer, prodrug, or salt thereof, comprises the structure of Formula (VIII′-a):

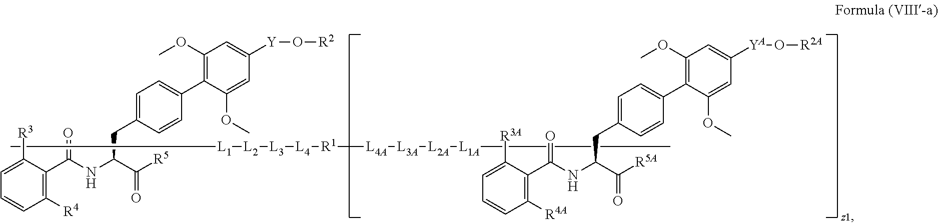

wherein R¹, R², R³, R⁴, R⁵, Y, R²A, R^{3A}, R^{4A}, R^{5A}, Y^{A}, z1, L₁, L₂, L₃, L₄, L_{1A}, L_{2A}, L_{3A}, and L_{4A} are as defined herein.

In some embodiments, the compound, or a stereoisomer, tautomer, prodrug, or salt thereof, comprises the structure of Formula (VIII):

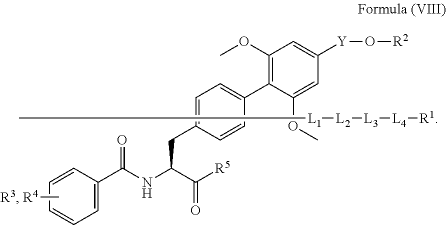

wherein R¹, R², R³, R⁴, R⁵, Y, L₁, L₂, L₃, and L₄, are as defined herein.

In some embodiments, the compound, or a stereoisomer, tautomer, prodrug, or salt thereof, comprises the structure of Formula (VIII-a):

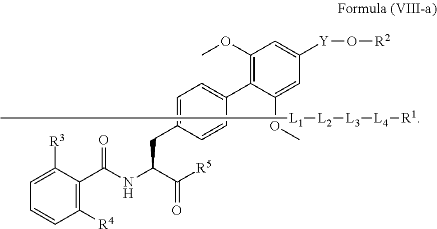

wherein R¹, R², R³, R⁴, Y, L₁, L₂, L₃, and L₄, are as defined herein.

In some embodiments, the compound, or a stereoisomer, tautomer, prodrug, or salt thereof, comprises the structure of Formula (VIII-a-1):

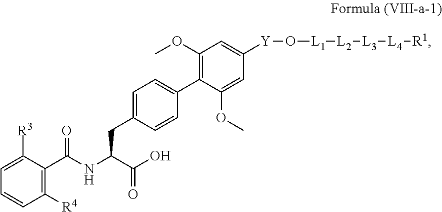

wherein R¹, R², R³, R⁴, Y, L₁, L₂, L₃, and L₄, are as defined herein.

In some embodiments, the compound, or a stereoisomer, tautomer, prodrug, or salt thereof, comprises the structure of Formula (VIII-a-2):

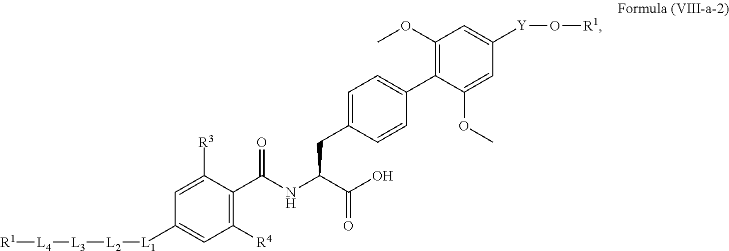

wherein R¹, R², R³, R⁴, Y, L₁, L₂, L₃, and L₄, are as defined herein.

In some embodiments, the compound comprises the structure of Formula (VIII-a-3):

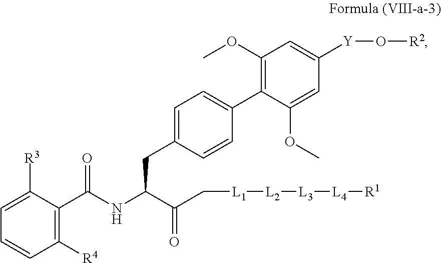

wherein R¹, R², R³, R⁴, Y, L₁, L₂, L₃, and L₄ are as defined herein.

In some embodiments, the compound, or a stereoisomer, tautomer, prodrug, or salt thereof, comprises the structure of Formula (IX′):

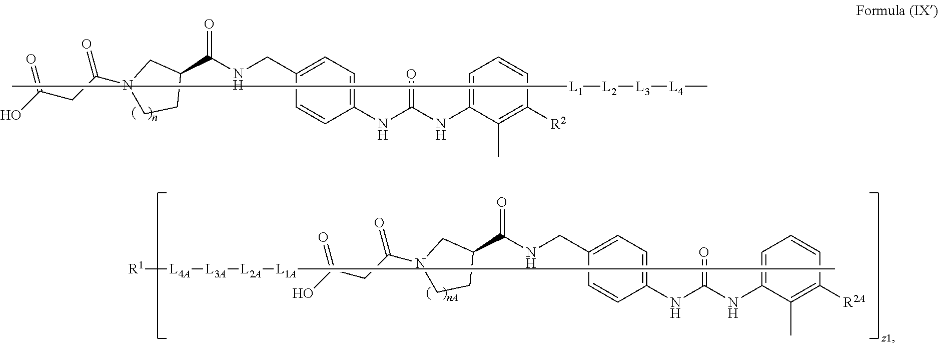

wherein
each of R² and R^{2A} is independently H, —OH, —NH₂, —NHR³, —OR³, or —CONHR³;
each instance of R³ is independently H, polyethylene glycol, optionally substituted alkyl, optionally substituted heteroalkyl, optionally substituted cycloalkyl, or optionally substituted heteroaryl;
each of n and n^{A} is independently 1 or 2; and
R¹, z1, L₁, L₂, L₃, L₄, L_{1A}, L_{2A}, L_{3A}, and L_{4A} are as defined herein.

In some embodiments, the compound, or a stereoisomer, tautomer, prodrug, or salt thereof, comprises the structure of Formula (IX):

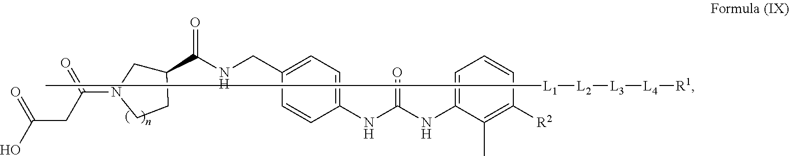

wherein R¹, R², n, L₁, L₂, L₃, and L₄ are as defined herein.

In some embodiments, the compound, or a stereoisomer, tautomer, prodrug, or salt thereof, comprises the structure of Formula (IX-a):

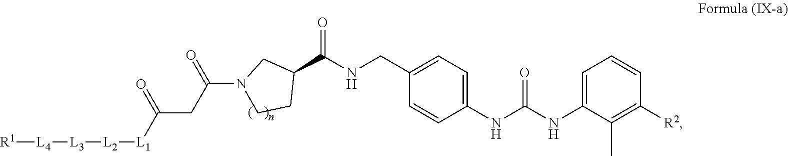

wherein R¹, R², n, L₁, L₂, L₃, and L₄ are as defined herein.

In some embodiments, the compound comprises the structure of Formula (IX-b):

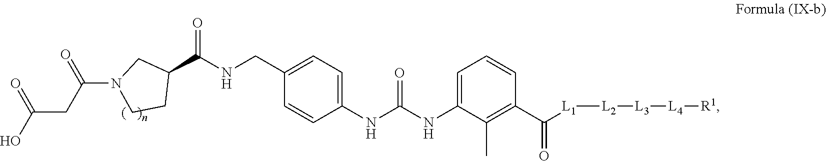

wherein R¹, n, L₁, L₂, L₃, and L₄ are as defined herein.

In some embodiments, the compound, or a stereoisomer, tautomer, prodrug, or salt thereof, comprises the structure of Formula (X′):

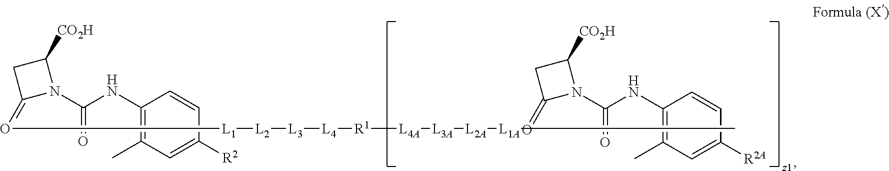

wherein
R² and R^{2A} are each independently H, —CH₂OR³, —(CH₂)₂OR³, —CH₂NHCOR³, or —OR³; and each instance of R³ is independently H, polyethylene glycol, optionally substituted alkyl, optionally substituted heteroalkyl, optionally substituted cycloalkyl, or optionally substituted heteroaryl; and
R¹, z¹, L₁, L₂, L₃, L₄, L_{1A}, L_{2A}, L_{3A}, and L_{4A} are as defined herein.

In some embodiments, the compound, or a stereoisomer, tautomer, prodrug, or salt thereof, comprises the structure of Formula (X):

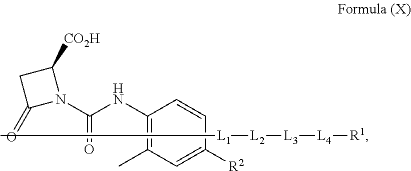

wherein R¹, R², L₁, L₂, L₃, and L₄ are as defined herein.

In some embodiments, the compound, or a stereoisomer, tautomer, prodrug, or salt thereof, comprises the structure of Formula (X-a):

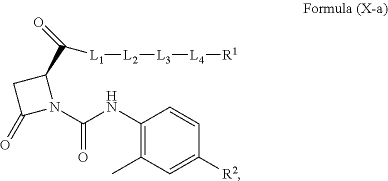

wherein R¹, R², L₁, L₂, L₃, and L₄ are as defined herein.

In some embodiments, the compound, or a stereoisomer, tautomer, prodrug, or salt thereof, comprises the structure of Formula (X-b):

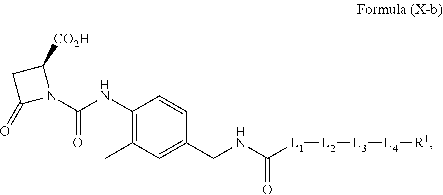

wherein R¹, L₁, L₂, L₃, and L₄ are as defined herein.

In some embodiments, the compound, or a stereoisomer, tautomer, prodrug, or salt thereof, comprises the structure of Formula (XI′):

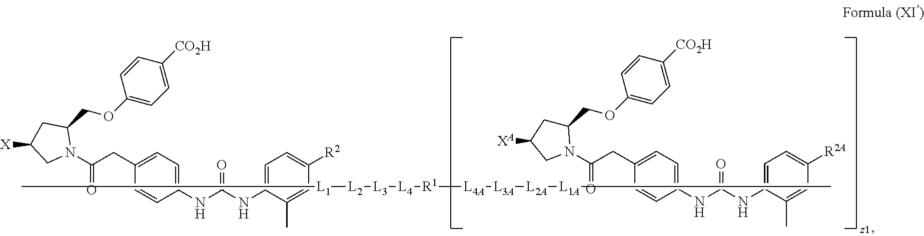

wherein
each of R² and R^{2A} is independently H, —CONHR³, —CH₂OR³, —(CH₂)₂OR³, —CH₂NHCOR³, or —OR³;
each instance of R³ is independently H, polyethylene glycol, optionally substituted alkyl, optionally substituted heteroalkyl, optionally substituted cycloalkyl, or optionally substituted heteroaryl;
each of X and X^{A} are independently H or halogen; and
R¹, z¹, L₁, L₂, L₃, L₄, L_{1A}, L_{2A}, L_{3A}, and L_{4A} are as defined herein.

In some embodiments, the compound, or a stereoisomer, tautomer, prodrug, or salt thereof, comprises the structure of Formula (XI):

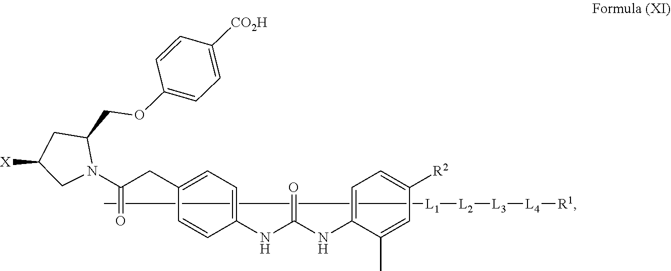

wherein R¹, R², X, L₁, L₂, L₃, and L₄ are as defined herein.

In some embodiments, the compound, or a stereoisomer, tautomer, prodrug, or salt thereof, comprises the structure of Formula (XI-a):

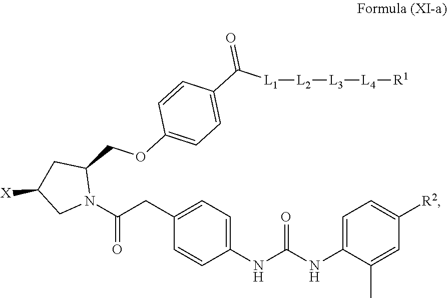

wherein R¹, R², X, L₁, L₂, L₃, and L₄ are as defined herein.

The compound, or a stereoisomer, tautomer, prodrug, or salt thereof, wherein the compound comprises the structure of Formula (XI-b):

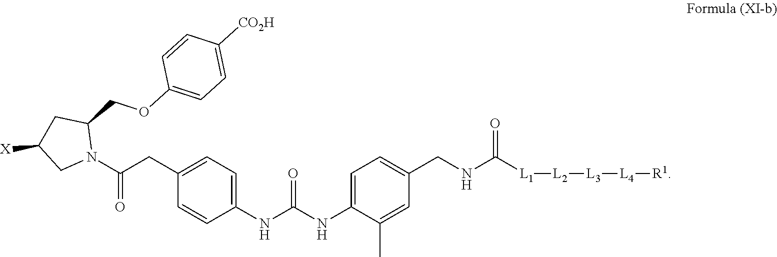

wherein R¹, X, L₁, L₂, L₃, and L₄ are as defined herein.

In some embodiments, the compound, or a stereoisomer, tautomer, prodrug, or salt thereof, comprises the structure of Formula (XII′):

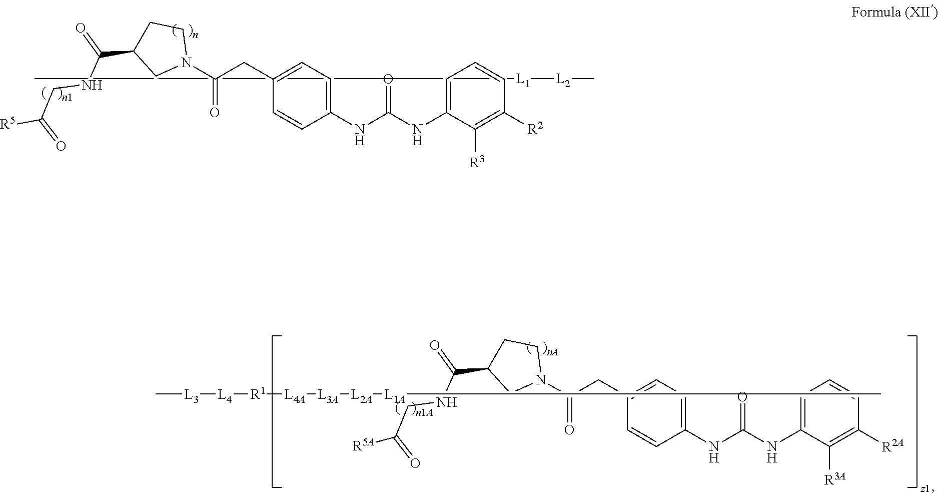

wherein
each of R² and R^{2A} is independently H, —CONHR⁴, —CH₂OR⁴, —(CH₂)₂OR⁴, —CH₂NHCOR⁴, or —OR⁴;
each of R³ and R^{3A} is independently H, optionally substituted alkyl, or optionally substituted cycloalkyl;
each instance of R⁴ is independently H, polyethylene glycol, optionally substituted alkyl, optionally substituted heteroalkyl, optionally substituted cycloalkyl, or optionally substituted heteroaryl;
each of R⁵ and R^{5A} is independently —OH or absent;
each instance of n and n^{A} is independently 0, 1, 2, or 3;
each instance of n1 and n^{1A} is independently 1, 2, or 3; and
R¹, z¹, L₁, L₂, L₃, L₄, L₁A, L_{2A}, L_{3A}, and L_{4A} are as defined herein.

In some embodiments, the compound, or a stereoisomer, tautomer, prodrug, or salt thereof, comprises the structure of Formula (XII):

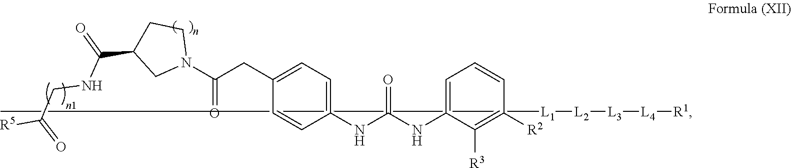

wherein R¹, R², R³, R⁴, R⁵, n, n1, L₁, L₂, L₃, and L₄ are as defined herein.

In some embodiments, the compound, or a stereoisomer, tautomer, prodrug, or salt thereof, comprises the structure of Formula (XII-a):

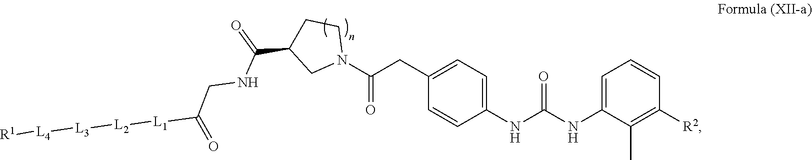

wherein R¹, R², R⁴, n, L₁, L₂, L₃, and L₄ are as defined herein.

In some embodiments, the compound, or a stereoisomer, tautomer, prodrug, or salt thereof, comprises the structure of Formula (XII-b):

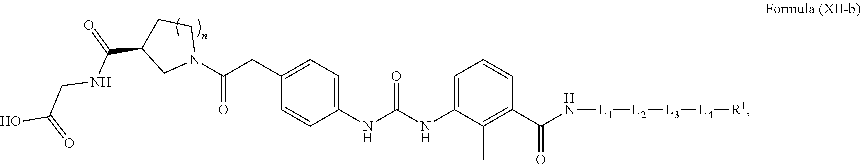

wherein R¹, n, L₁, L₂, L₃, and L₄ are as defined herein.

In some embodiments, the compound, or a stereoisomer, tautomer, prodrug, or salt thereof, comprises the structure of Formula (XIII′):

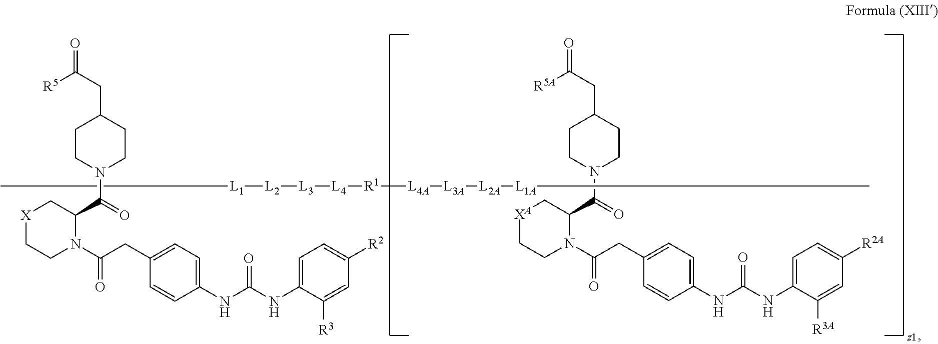

wherein
each of R² and R^{2A} is independently H, —CONHR⁴, —CH₂OR⁴, —(CH₂)₂OR⁴, —CH₂NHCOR⁴, or —OR⁴;
each of R³ and R^{3A} is independently H, optionally substituted alkyl, or optionally substituted cycloalkyl;
each instance of R⁴ is independently H, polyethylene glycol, optionally substituted alkyl, optionally substituted heteroalkyl, optionally substituted cycloalkyl, or optionally substituted heteroaryl;
each of R⁵ and R^{5A} is independently —OH or absent;
each of X and X^{A} is independently H, optionally substituted CH₂, optionally substituted NH, or cycloalkyl; and
R¹, z1, L₁, L₂, L₃, L₄, L_{1A}, L_{2A}, L_{3A}, and L_{4A} are as defined herein.

In some embodiments, the compound, or a stereoisomer, tautomer, prodrug, or salt thereof, comprises the structure of Formula (XIII):

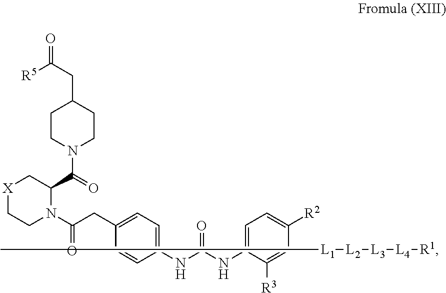

wherein R¹, R², R³, R⁴, R⁵, X, L₁, L₂, L₃, and L₄ are as defined herein.

In some embodiments, the compound, or a stereoisomer, tautomer, prodrug, or salt thereof, comprises the structure of Formula (XIII-a):

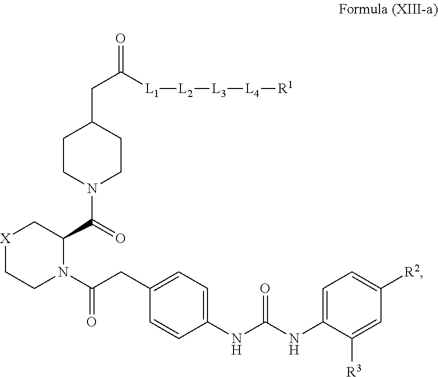

wherein R¹, R², R³, R⁴, X, L₁, L₂, L₃, and L₄ are as defined herein.

In some embodiments, the compound, or a stereoisomer, tautomer, prodrug, or salt thereof, comprises the structure of Formula (XIII-b):

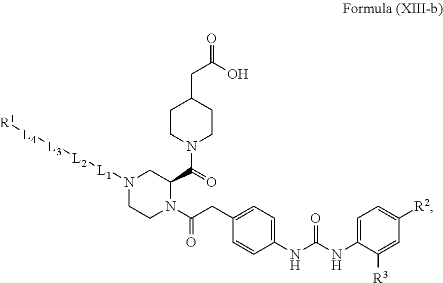

wherein R¹, R², R³, R⁴, X, L₁, L₂, L₃, and L₄ are as defined herein.

In some embodiments, the compound, or a stereoisomer, tautomer, prodrug, or salt thereof, comprises the structure of Formula (XIII-c):

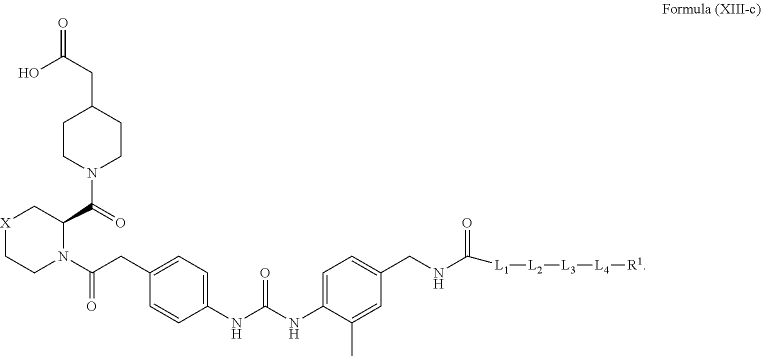

wherein R¹, X, L₁, L₂, L₃, and L₄ are as defined herein.

In some embodiments, the compound, or a stereoisomer, tautomer, prodrug, or salt thereof, comprises the structure of Formula (XIV′):

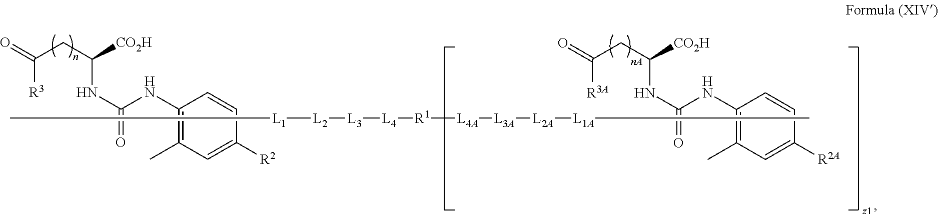

wherein
each of R² and R^{2A} is independently H, —CH₂OR⁴, —(CH₂)₂OR⁴, —CH₂NHCOR⁴, or —OR⁴;
each of R³ and R^{3A} is independently H, —OH, —NH₂, —NHR⁵, or —OR⁵;
each instance of R⁴ is independently H, polyethylene glycol, optionally substituted alkyl, optionally substituted heteroalkyl, optionally substituted cycloalkyl, or optionally substituted heteroaryl;
each instance of R⁵ is independently H, polyethylene glycol, optionally substituted alkyl, optionally substituted heteroalkyl, optionally substituted cycloalkyl, or optionally substituted heteroaryl;
each of n and n^{A} is independently 1, 2, or 3; R¹, z1, L₁, L₂, L₃, L₄, L_{4A}, L_{2A}, L_{3A}, and L_{4A} are as defined herein.

In some embodiments, the compound, or a stereoisomer, tautomer, prodrug, or salt thereof, comprises the structure of Formula (XIV):

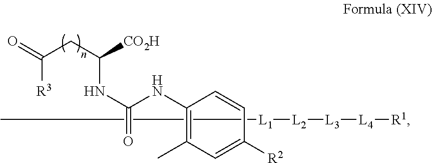

wherein R¹, R², R³, R⁴, R⁵, n, L₁, L₂, L₃, and L₄ are as defined herein.

In some embodiments, the compound, or a stereoisomer, tautomer, prodrug, or salt thereof, comprises the structure of Formula (XIV-a):

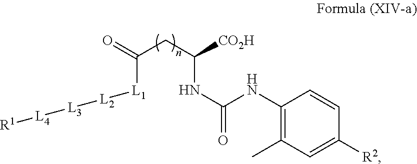

wherein R¹, R², n, L₁, L₂, L₃, and L₄ are as defined herein.

In some embodiments, the compound, or a stereoisomer, tautomer, prodrug, or salt thereof, comprises the structure of Formula (XIV-b):

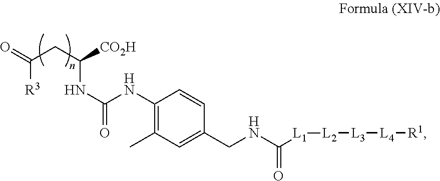

wherein R¹, R³, n, L₁, L₂, L₃, and L₄ are as defined herein.

In some embodiments, each of L₁, L₂, L₃, L₄, L_{1A}, L_{2A}, L_{3A}, and L_{4A} is independently absent, a bond, an optionally substituted alkyl linker, an optionally substituted polyethylene glycol (PEG) linker, an optionally substituted heteroalkyl linker, an optionally substituted heteroaryl linker, an optionally substituted saturated or partially unsaturated heterocycloalkyl linker, oxygen, optionally substituted nitrogen, an amide, a phosphodiester bond, or a phosphorothioate bond.

In certain embodiments, L₁ and/or L_{1A} is a bond.

In some embodiments, L₂ and/or L_{2A} is an optionally substituted PEG linker. In some embodiments, the PEG linker is two, three, four, five, six, seven, eight, nine, or ten PEG units in length. In certain embodiments, L₂ and/or L_{2A} comprises the structure

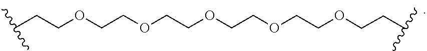 [SMILES: *CCOCCOCCOCCOCCOCC*]

In some embodiments, L₃ and/or L_{3A} is an optionally substituted heteroaryl linker. In some embodiments, L₃ and/or L_{3A} is an optionally substituted partially unsaturated heteroaryl linker. In

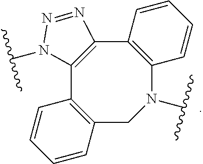 [SMILES: *N1Cc2ccccc2-c2c(nnn2*)-c2ccccc21]

certain embodiments, L₃ and/or L_{3A} comprises the structure

In some embodiments, L₄ and/or L_{4A} is an optionally substituted heteroalkyl linker. In some embodiments, the heteroalkyl linker is substituted with one or more ═O substituents. In some embodiments, the heteroalkyl linker comprises two substituents joined together to form an optionally substituted carbocyclyl ring. In certain embodiments, L₄ and/or L_{4A} comprises the structure

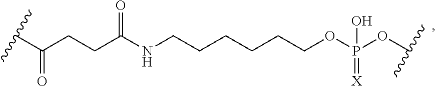

wherein X is O or S. In certain embodiments, L₄ and/or L_{4A} comprises the structure

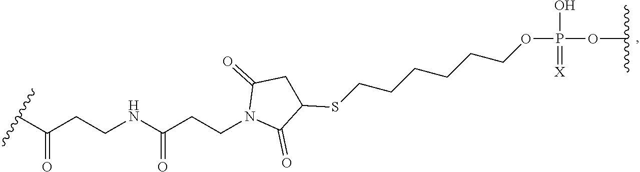

wherein X is O or S.

In certain embodiments, L₁, L₂, L₃, and L₄ and/or L_{1A}, L_{2A}, L_{3A}, and L_{4A} together comprise the structure

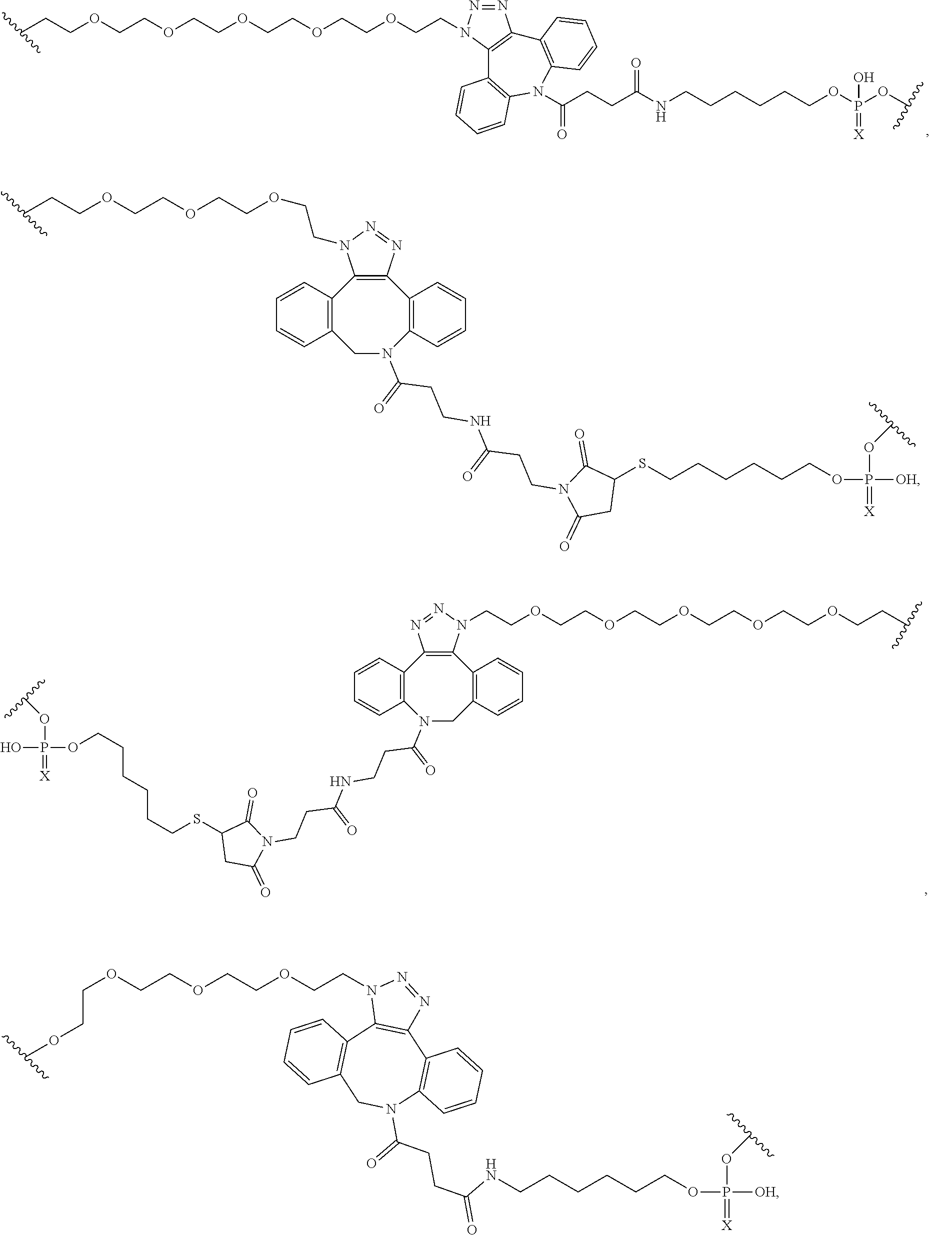

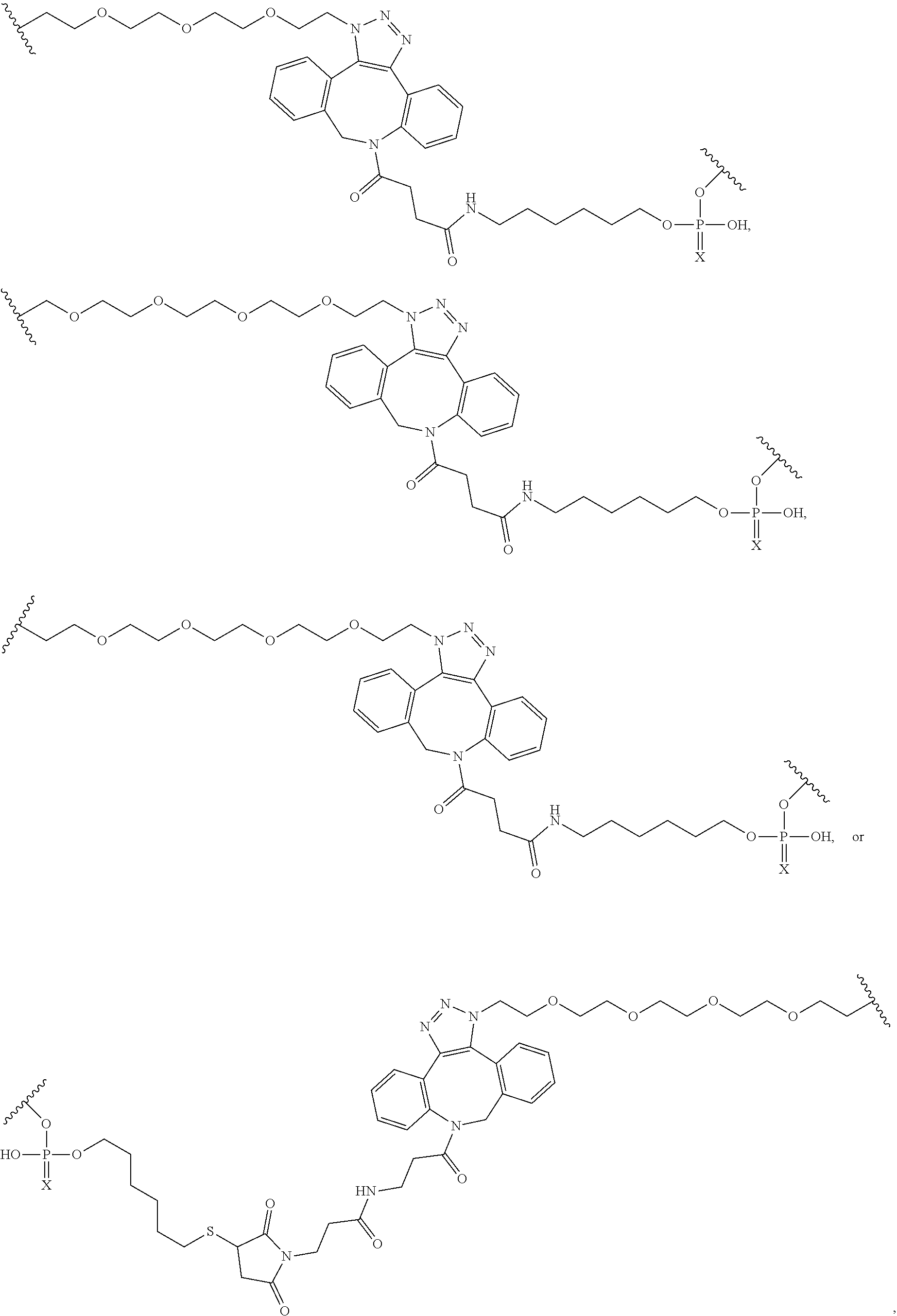

wherein X is O or S.

In certain embodiments, the compound comprises the structure:

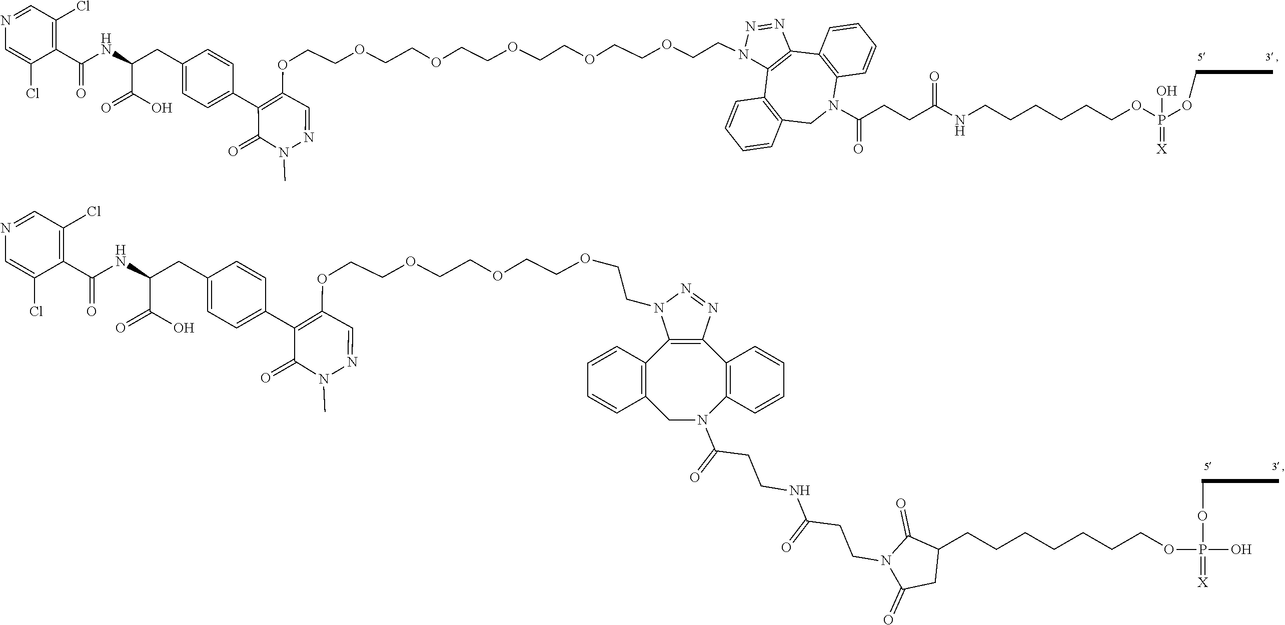

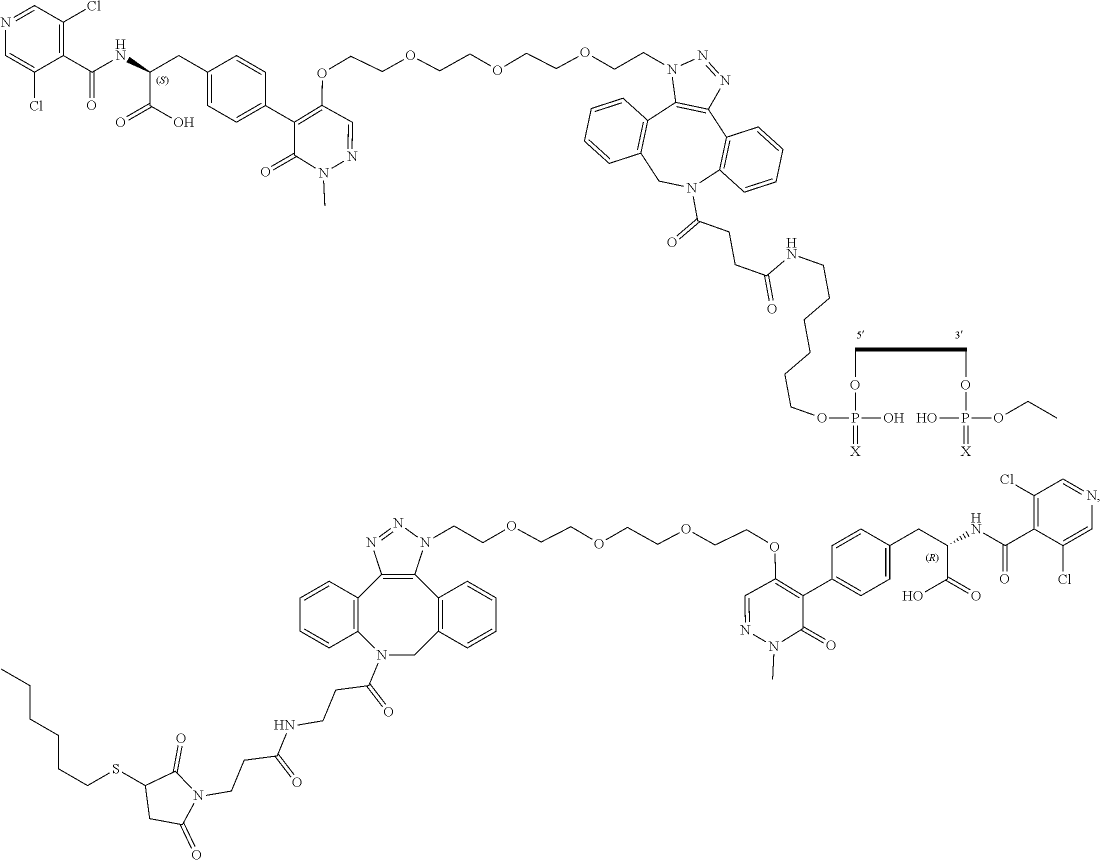

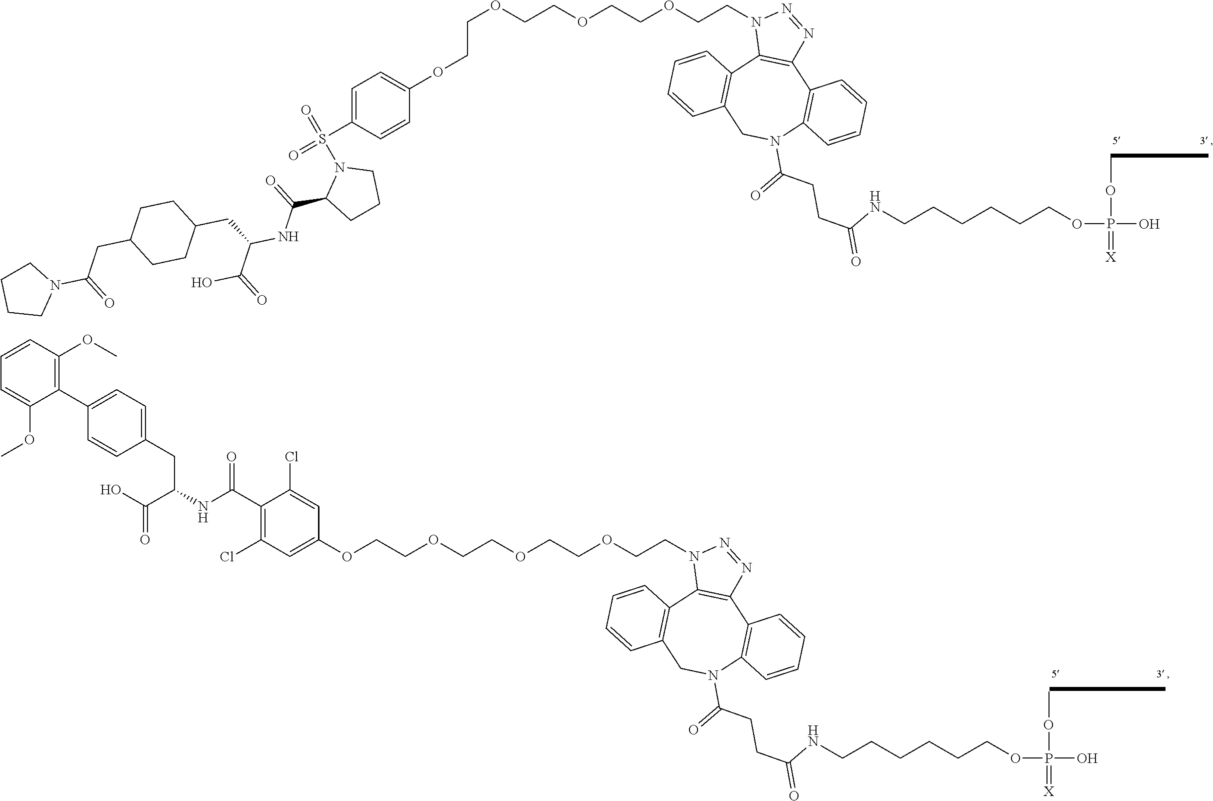

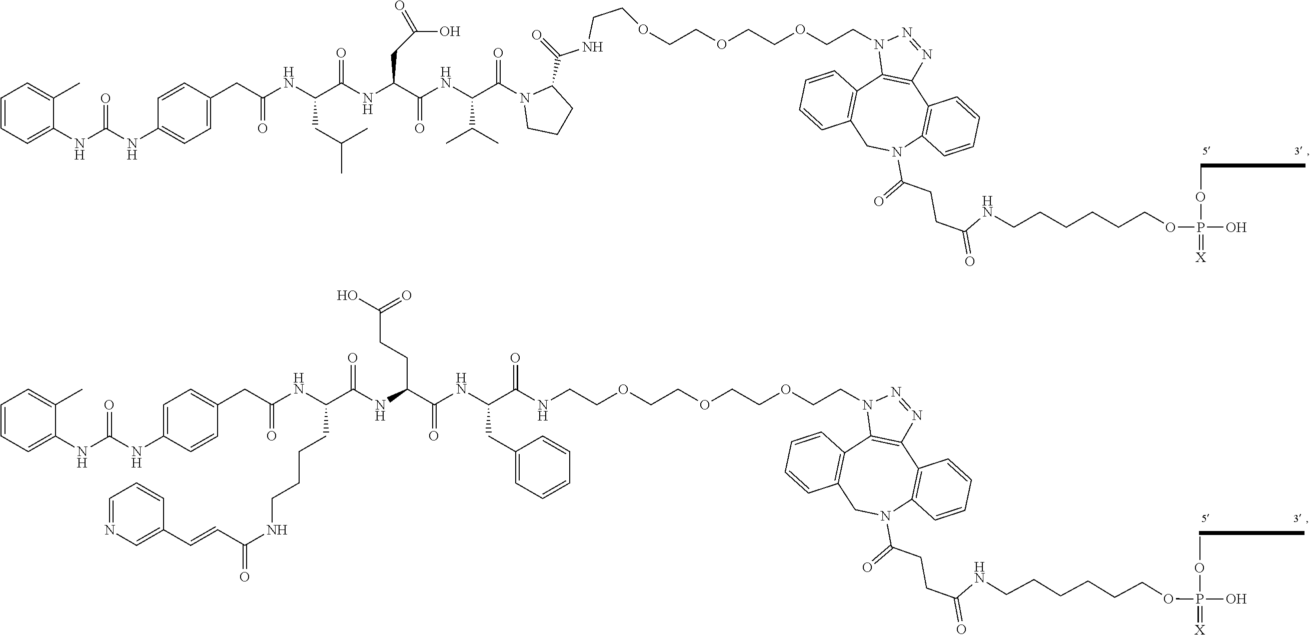

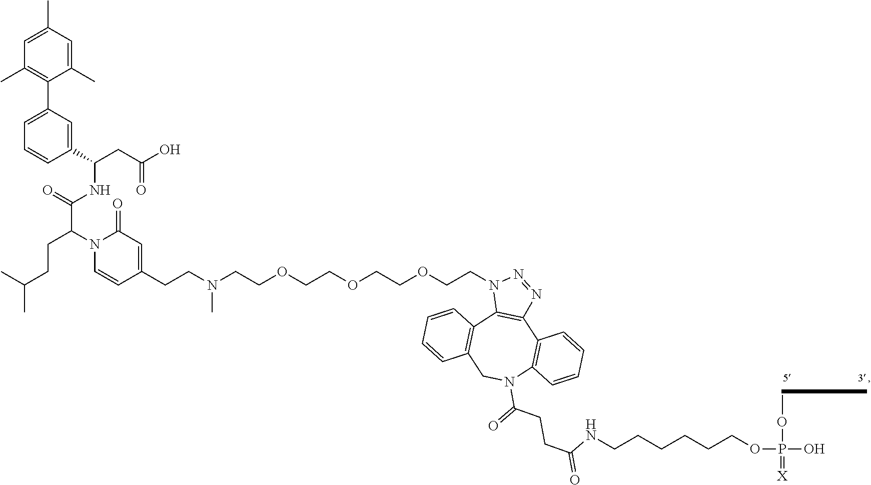

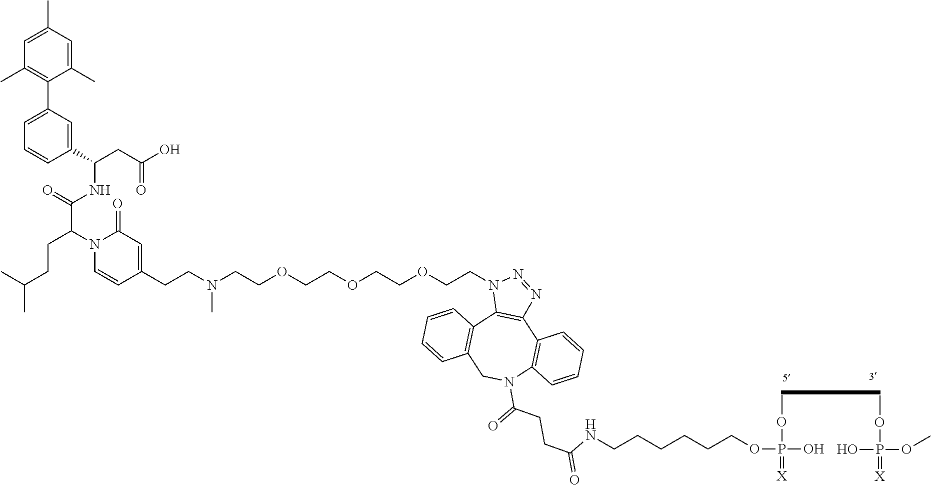

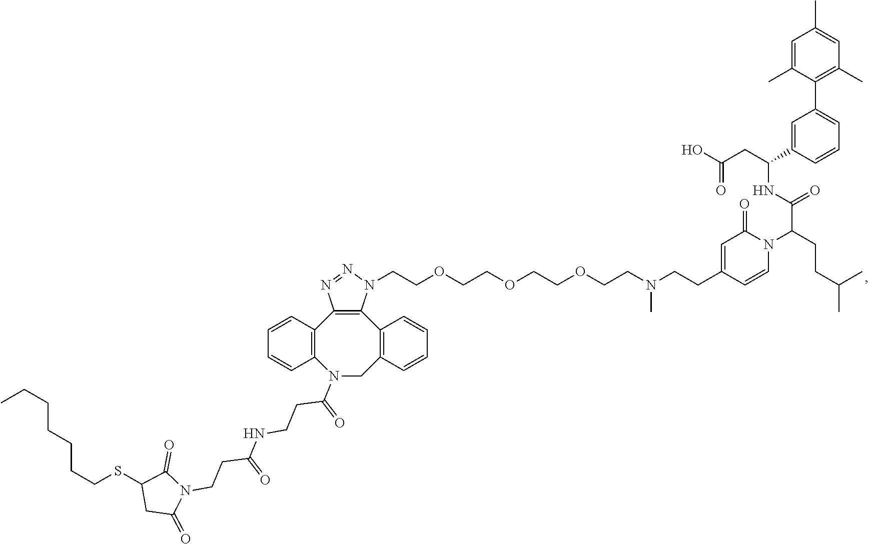

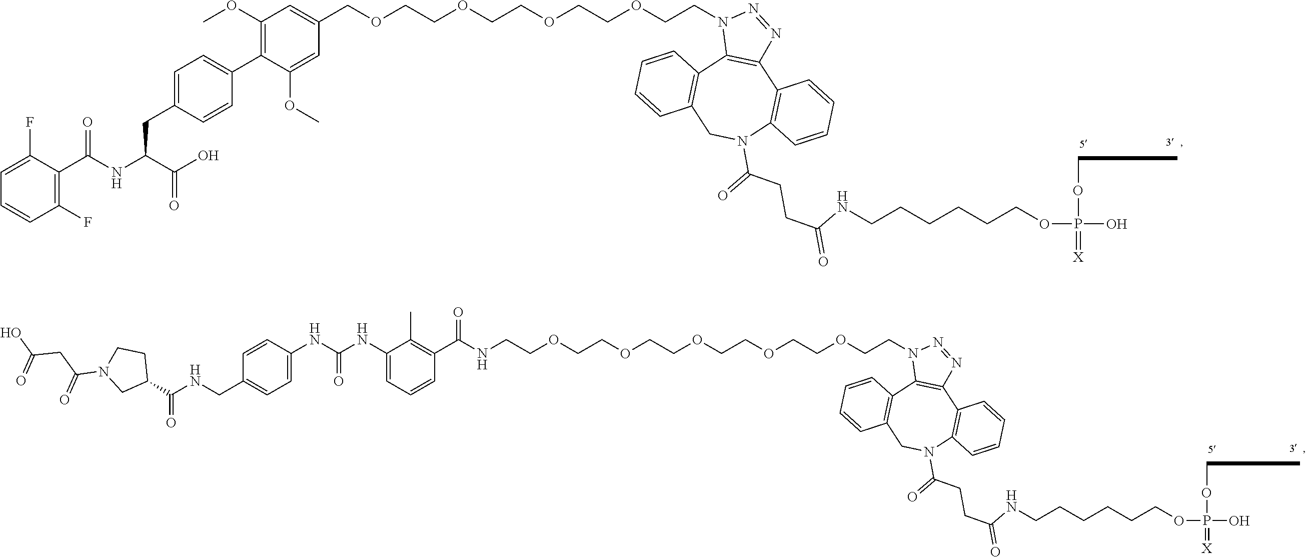

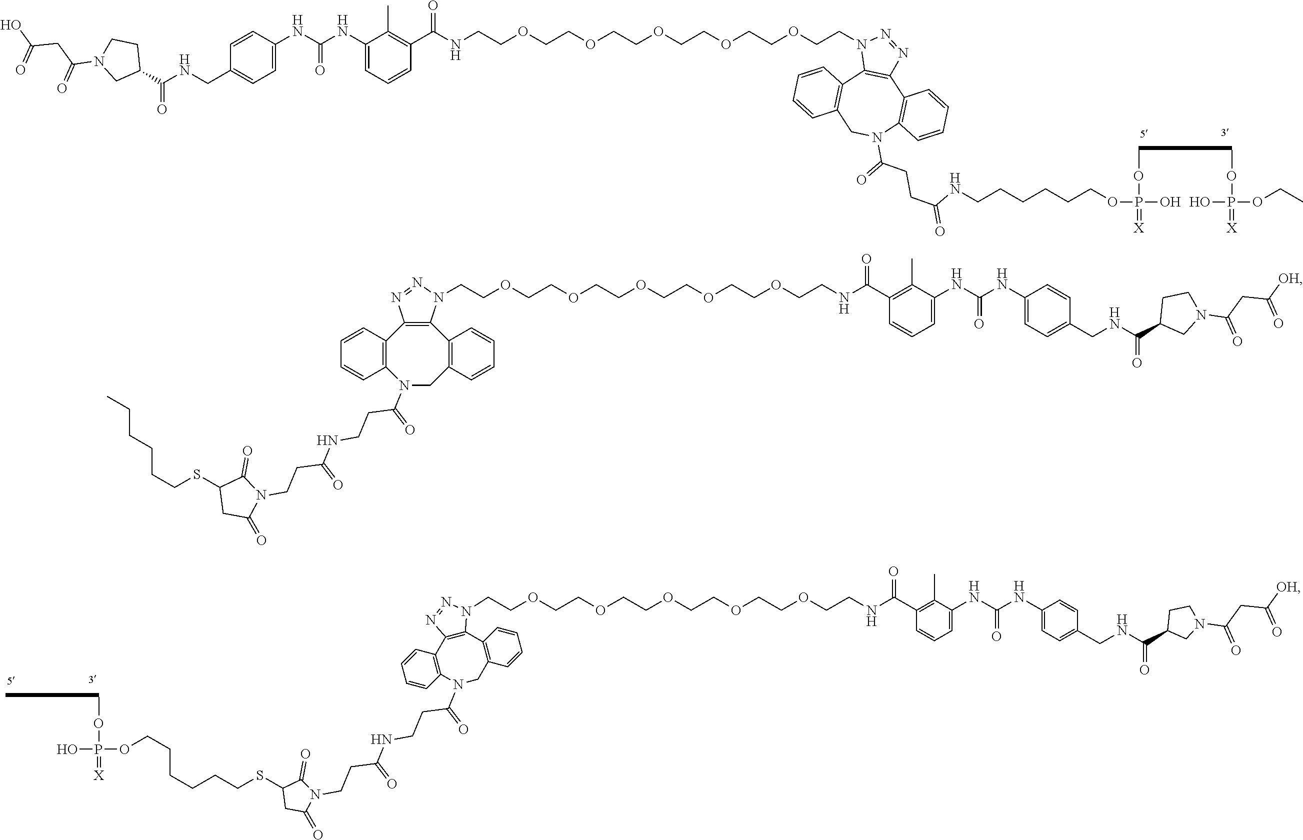

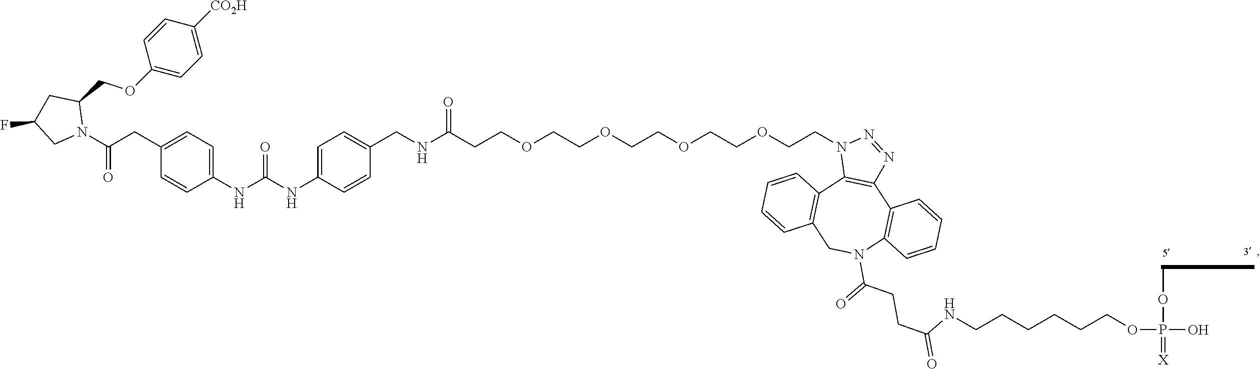

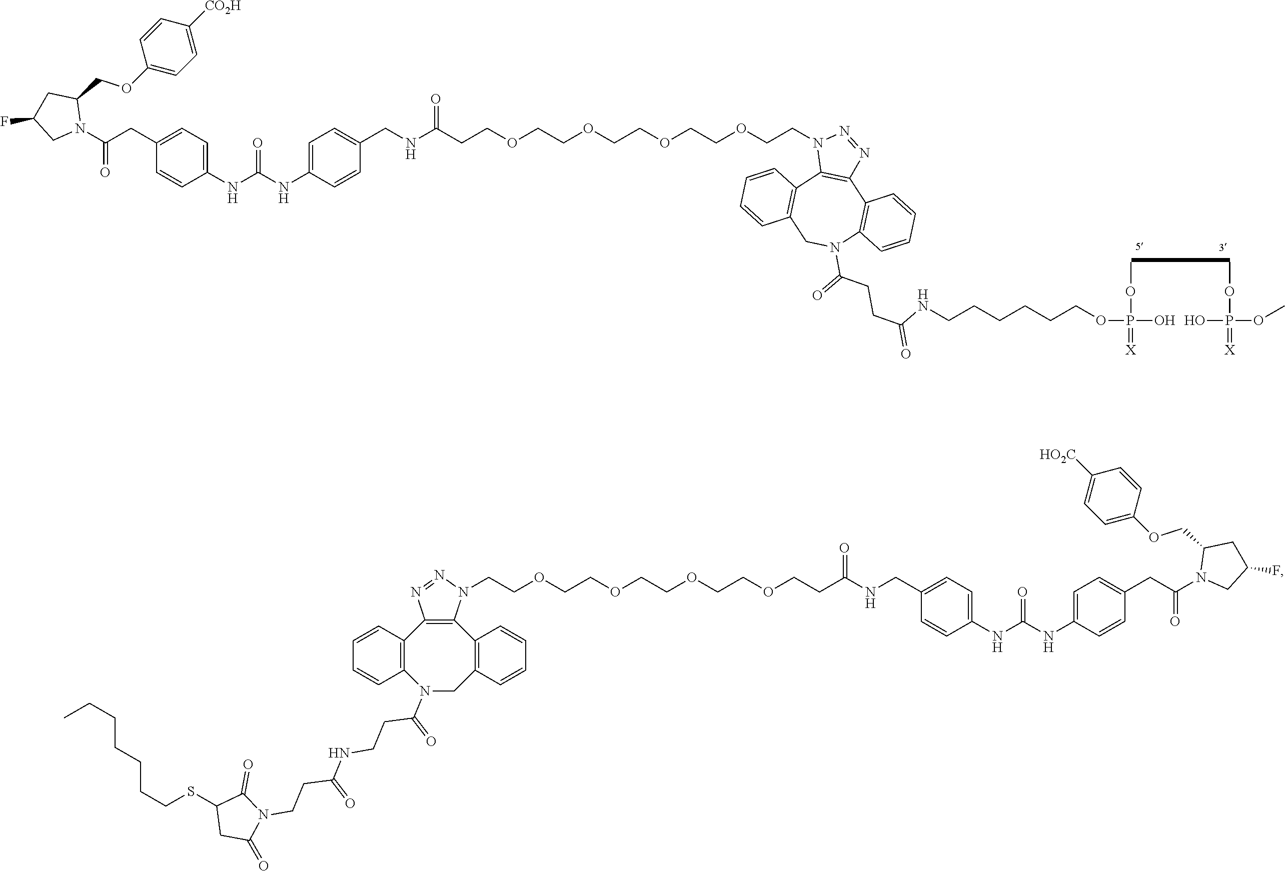

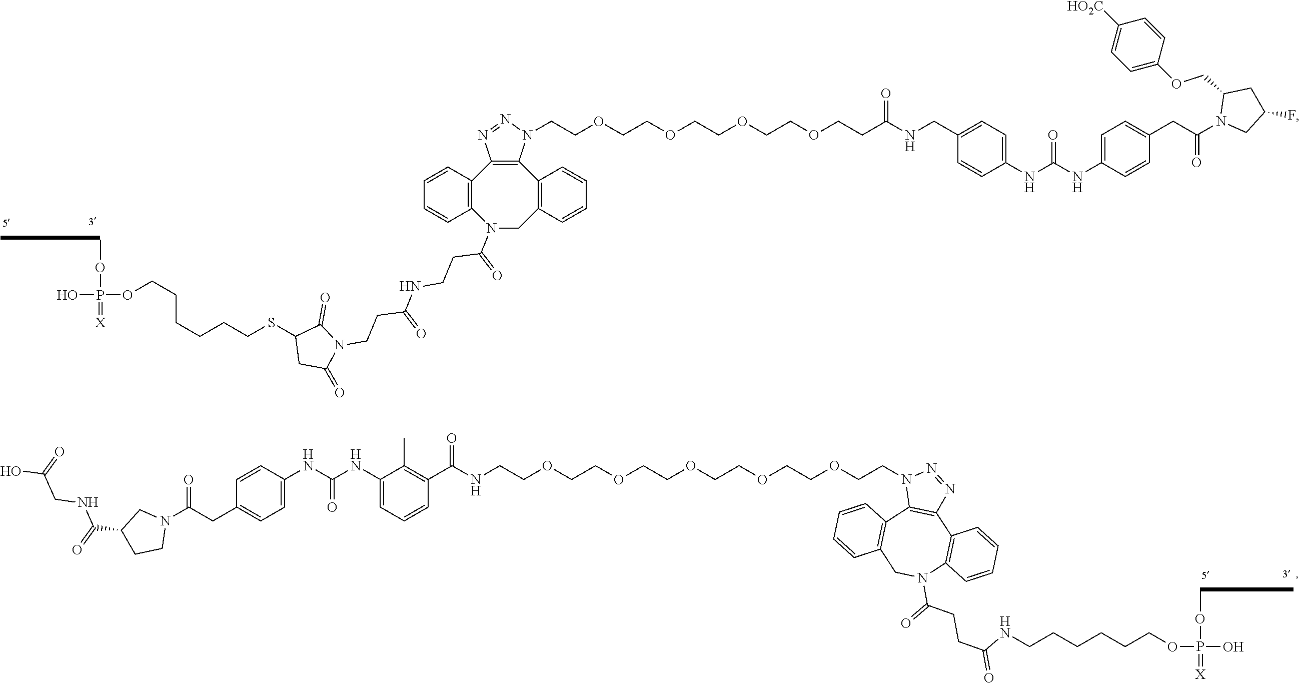

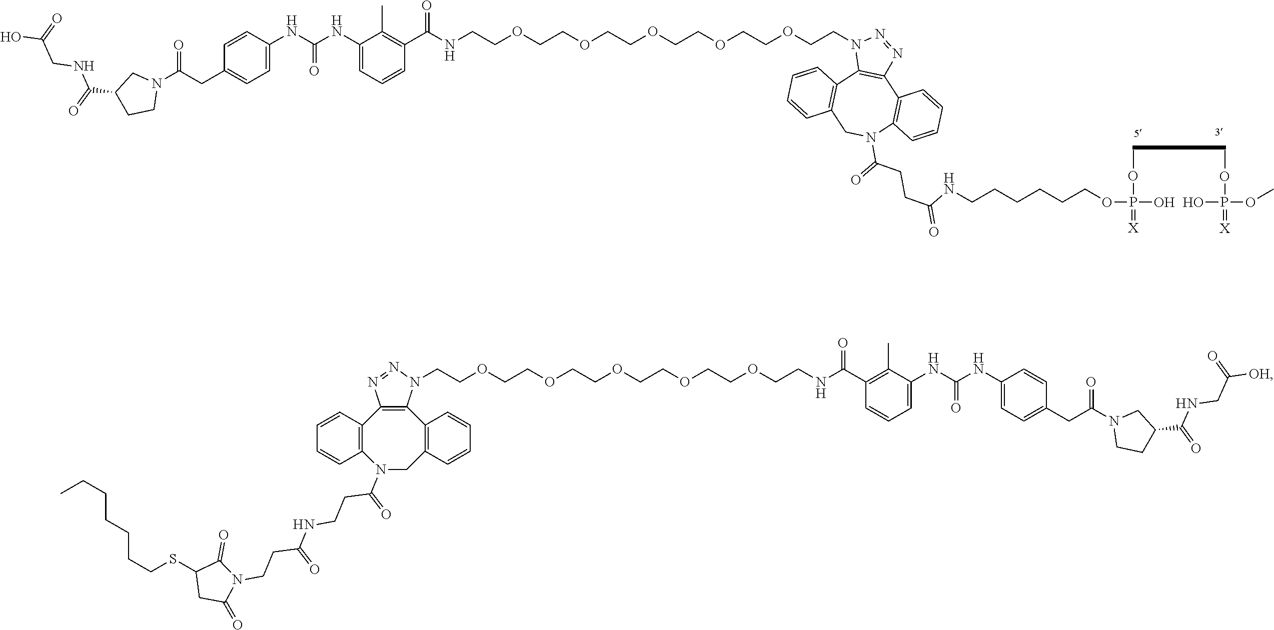

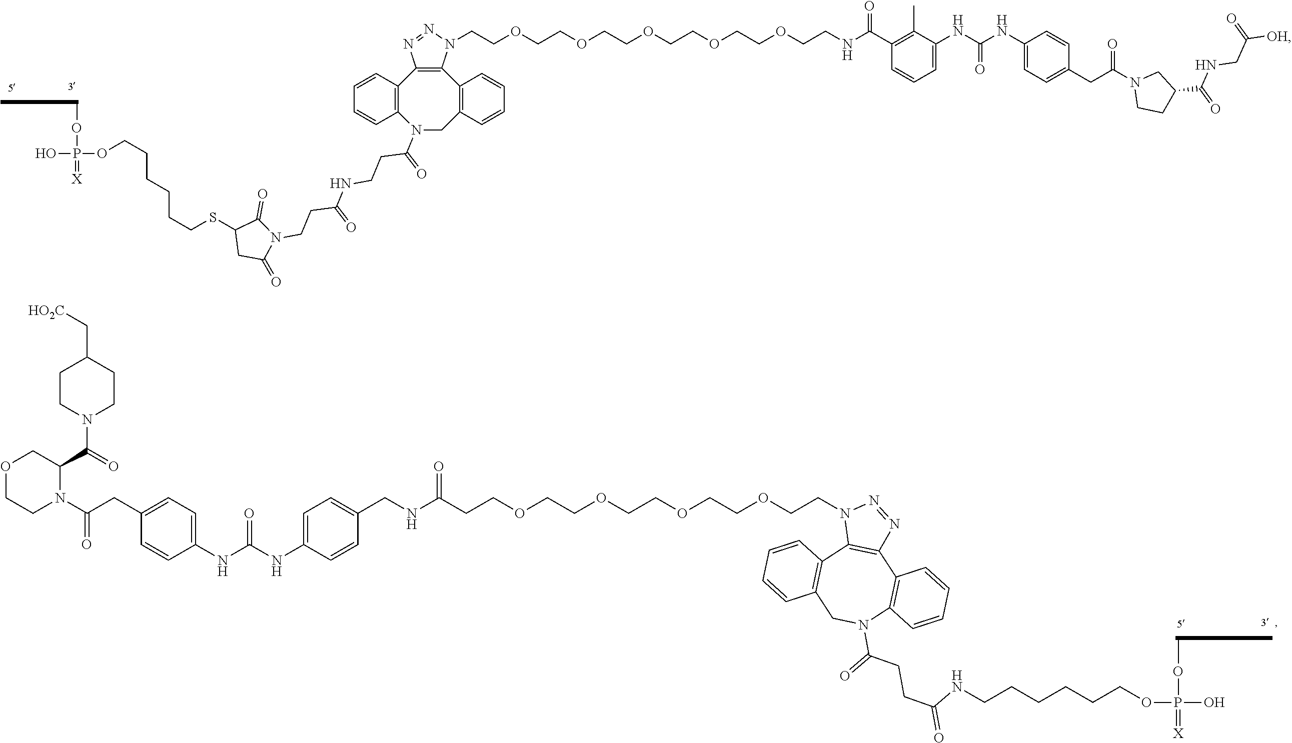

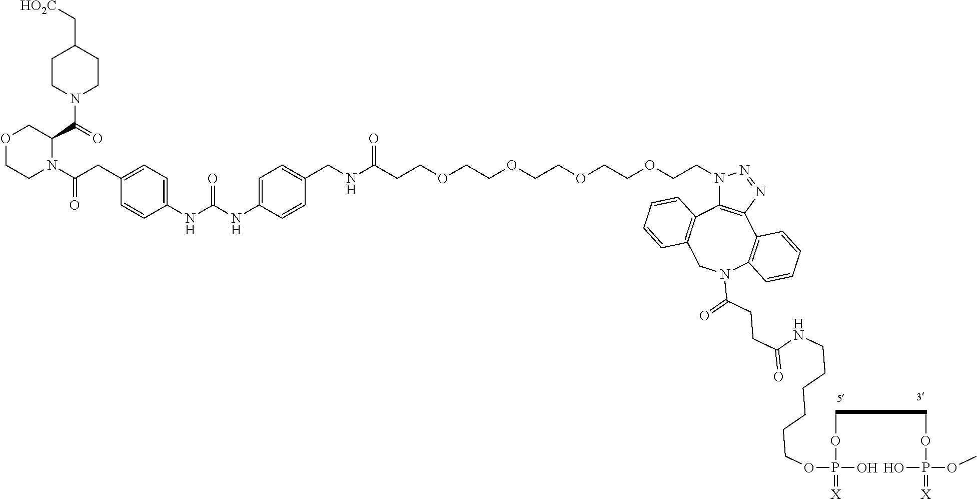

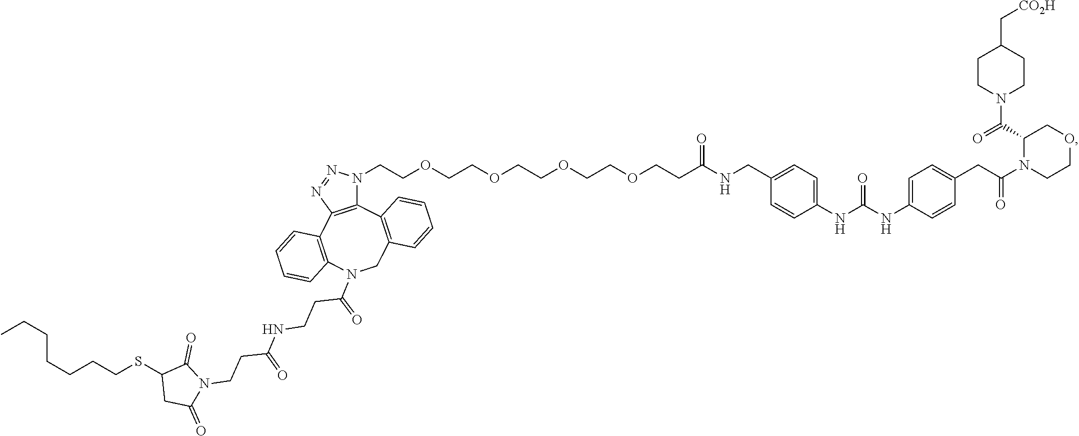

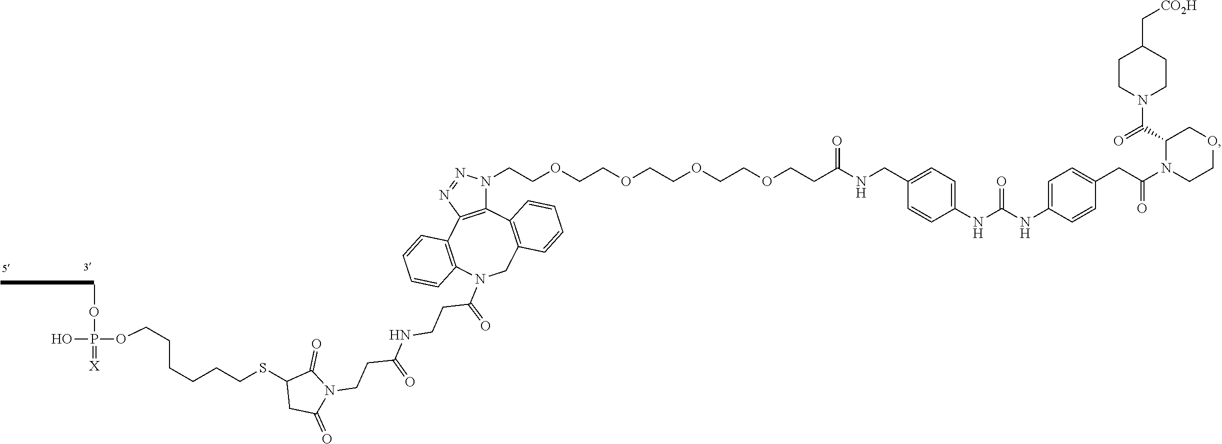

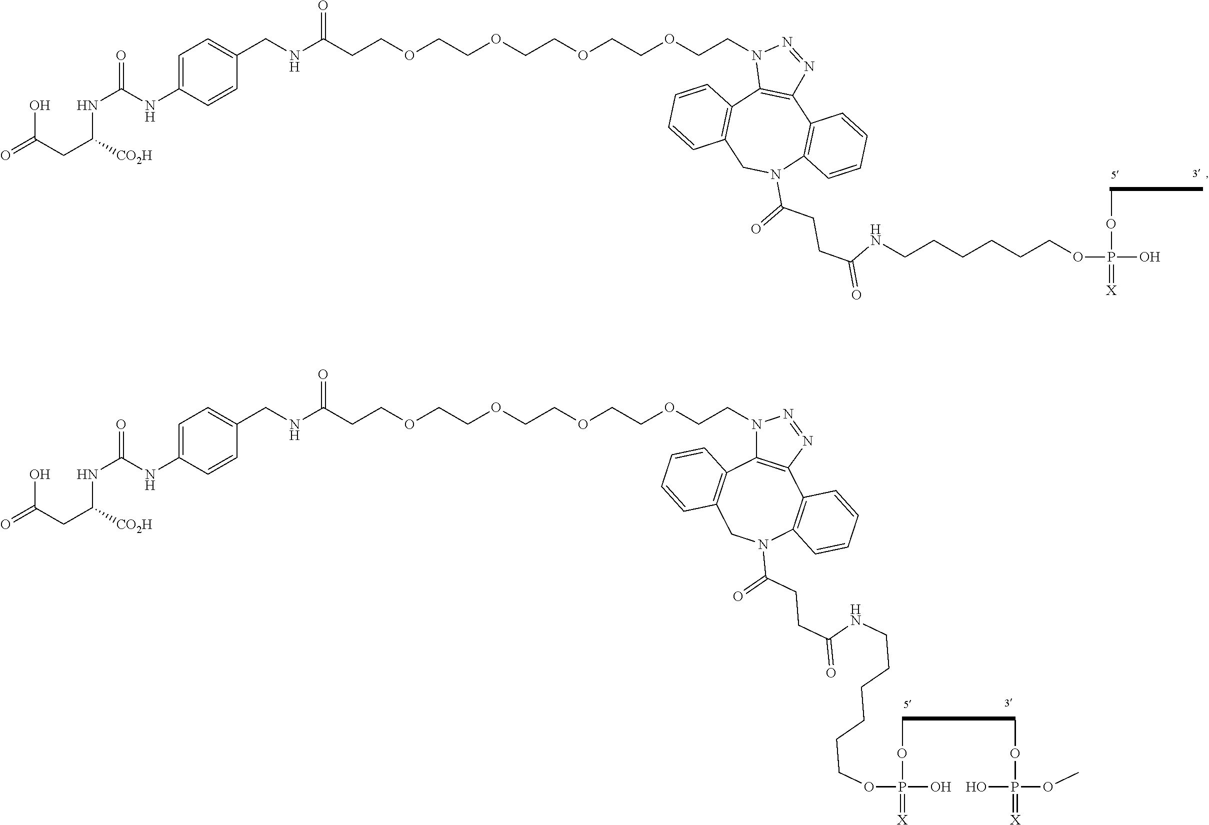

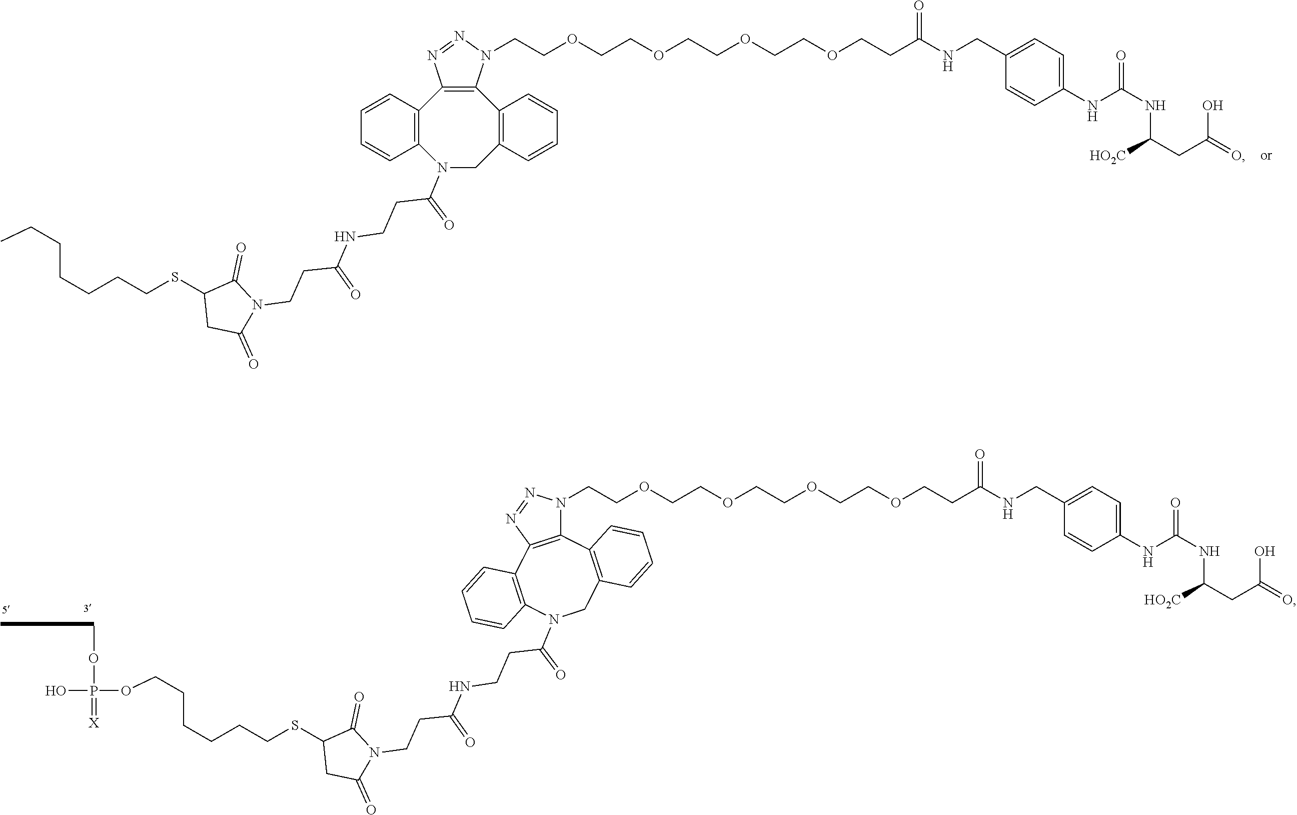

wherein X is O or S.

In certain embodiments, X is O. In certain embodiments, X is S.

In some embodiments, R¹ comprises an oligonucleotide. In some embodiments, the oligonucleotide is attached at its 5′ end. In some embodiments, the oligonucleotide is attached at its 3′ end. In some embodiments, the oligonucleotide is attached at an internal position on the oligonucleotide. In certain embodiments, the internal position is an internucleoside linkage. In some embodiments, R¹ comprises an oligonucleotide conjugated to one or more additional α₄β_{1/7} ligands. In some embodiments, the oligonucleotide is conjugated to two, three, four, five, or more than five additional α₄β_{1/7} ligands. In certain embodiments, the additional α₄β_{1/7} ligands are conjugated to the oligonucleotide at the 5′ end of the oligonucleotide, the 3′ end of the oligonucleotide, one or more internal positions on the oligonucleotide, or any combination thereof. In certain embodiments, the oligonucleotide is a modified oligonucleotide.

In another aspect, the present disclosure provides compositions comprising any of the compounds, or a stereoisomer, tautomer, prodrug, or salt thereof, disclosed herein, and a pharmaceutically acceptable excipient.

In another aspect, the present disclosure provides methods for delivering a therapeutic oligonucleotide to the brain of a subject, comprising administration of any of the compounds, or a stereoisomer, tautomer, prodrug, or salt thereof, described herein, or any of the compositions described herein, to the subject. In some embodiments, the therapeutic oligonucleotide is delivered to one or more brain regions selected from the group consisting of the striatum, the cerebellum, the brain stem, the hippocampus, the frontal cortex, and the spinal cord.

In another aspect, the present disclosure provides methods for treating or ameliorating a disease, disorder, or symptom thereof in a subject, comprising administration of any of the compounds, or a stereoisomer, tautomer, prodrug, or salt thereof, disclosed herein, or any of the compositions disclosed herein, to the subject. In some embodiments, the disease, disorder, or symptom thereof is a central nervous system (CNS) disease, disorder, or symptom thereof. In certain embodiments, the disease, disorder, or symptom thereof is Alzheimer's disease, or a symptom thereof. In certain embodiments, the compound, or a stereoisomer, tautomer, prodrug, or salt thereof, is administered to the subject intrathecally.

In another aspect, the present disclosure provides methods for making any of the compounds, or a stereoisomer, tautomer, prodrug, or salt thereof, disclosed herein, comprising one or more compounds and chemical transformations described herein, including Examples 1-13.

### BRIEF DESCRIPTION OF THE DRAWINGS

FIG. 1 shows a ¹H NMR of compound 2 from Example 1.

FIG. 2 shows a ¹H NMR of compound 5 from Example 1.

FIG. 3 shows a ¹H NMR of compound 6 from Example 1.

FIG. 4 shows a ¹H NMR of compound 7 from Example 1.

FIG. 5 shows a ¹H NMR of compound 8 from Example 1.

FIG. 6 shows a ¹H NMR of compound 10 from Example 1.

FIGS. 7A-7C show characterization of compound 11 from Example 1. ¹H NMR (FIG. 7A), LC/MS (FIG. 7B), and mass spectrometry data (FIG. 7C) are shown.

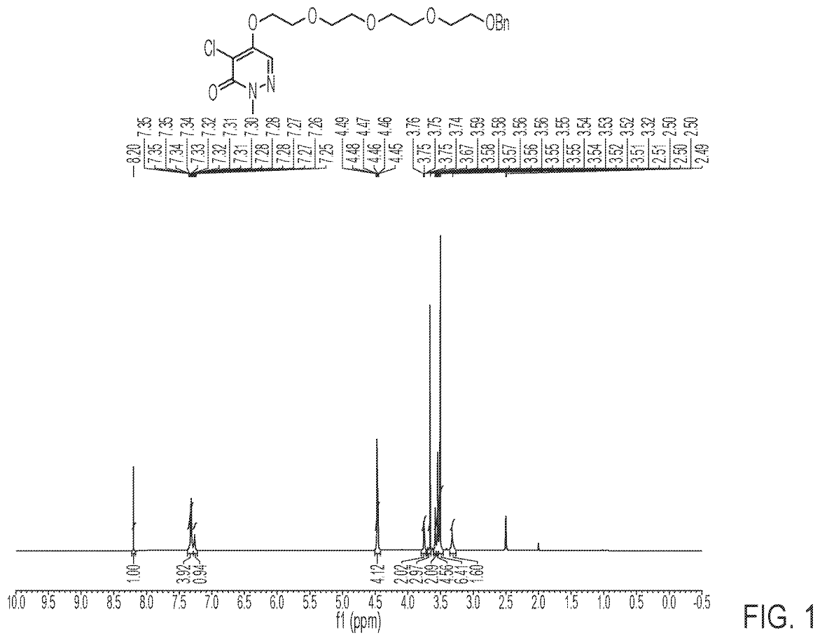

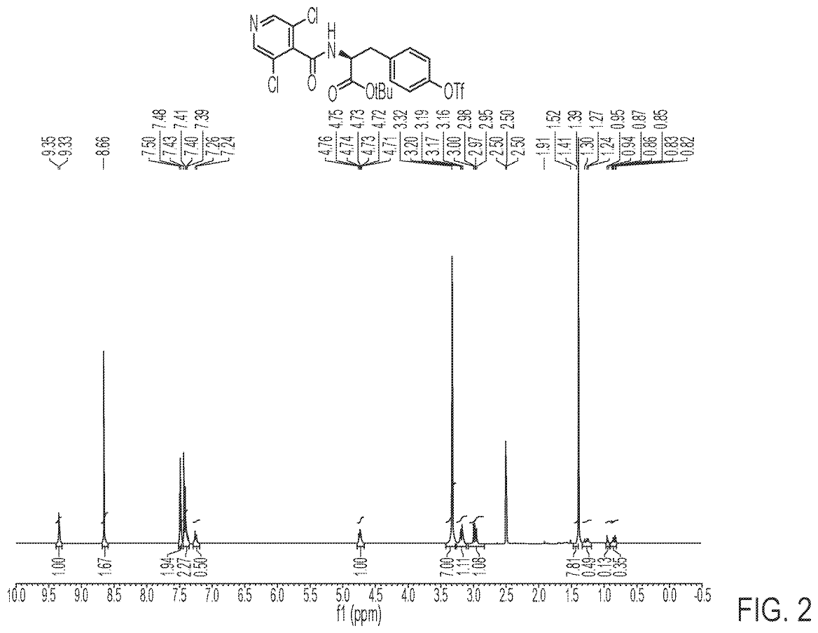

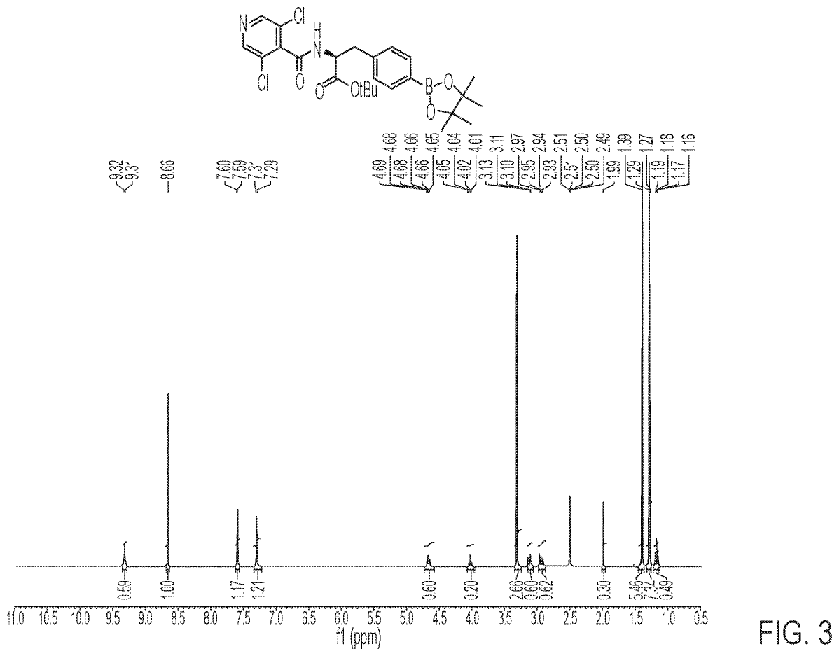

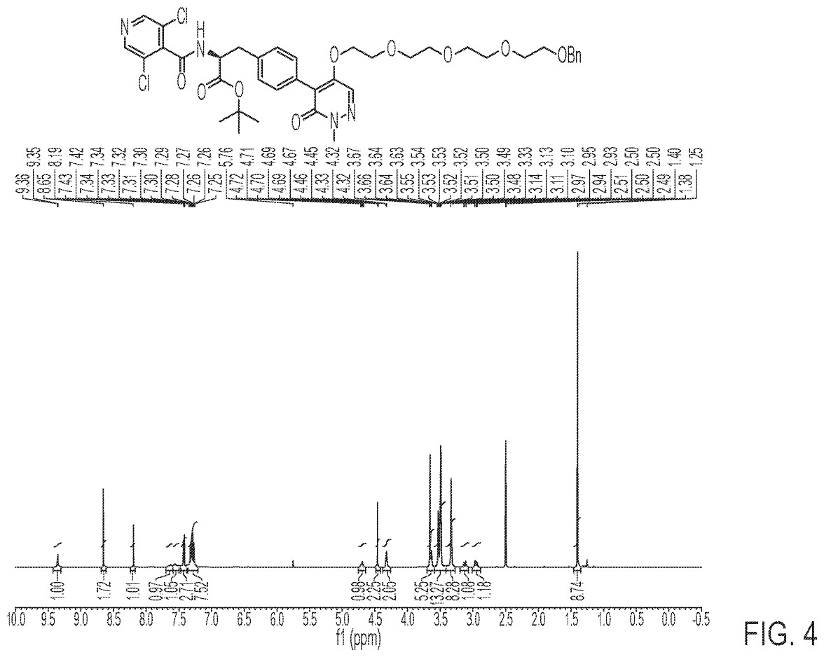

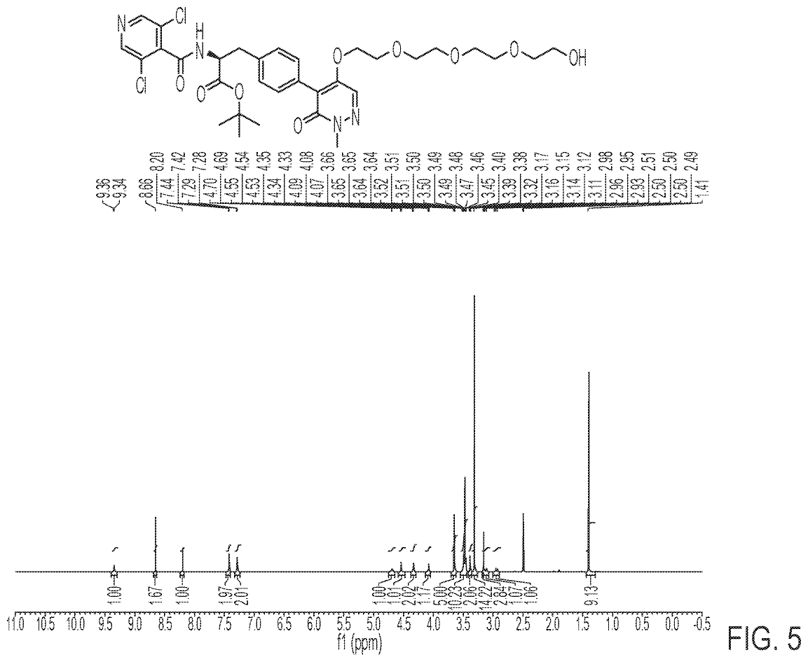

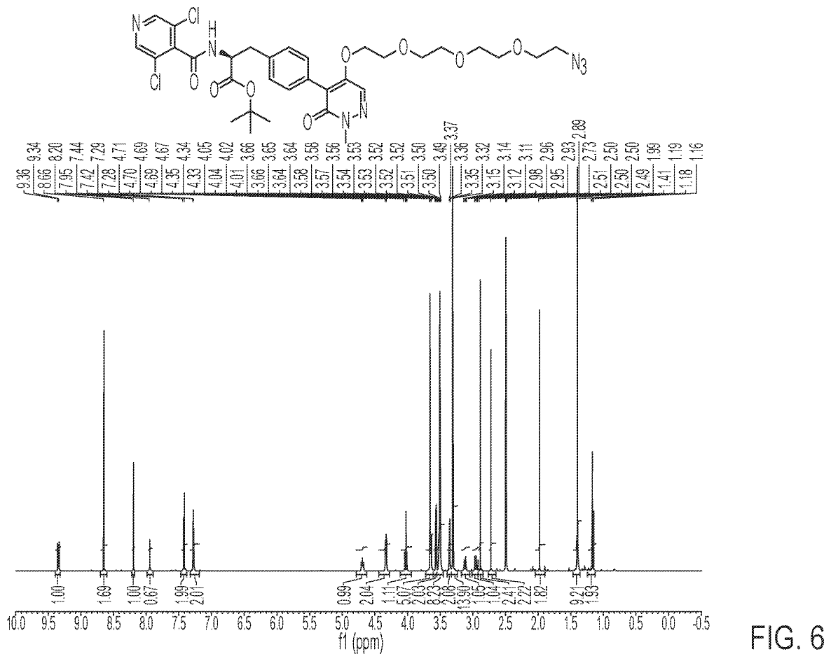

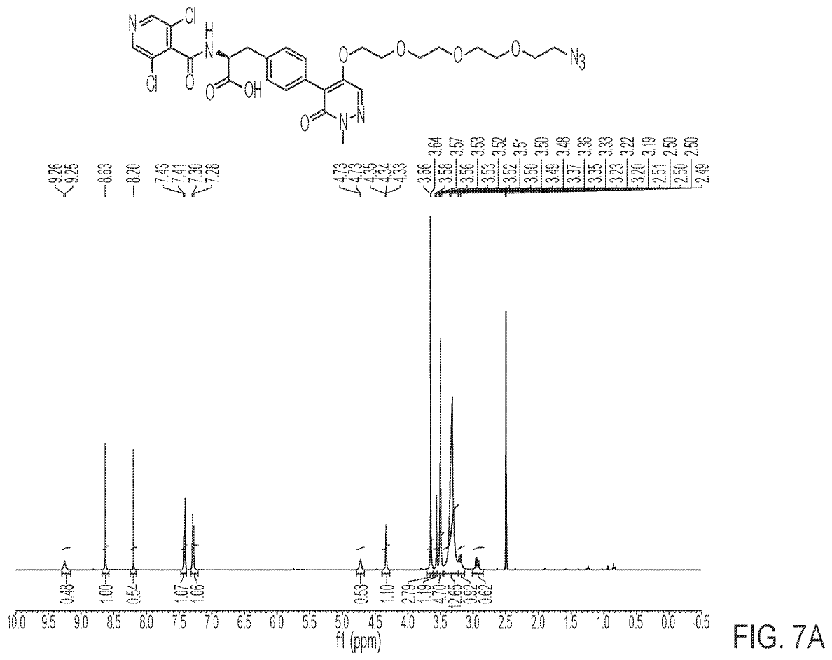

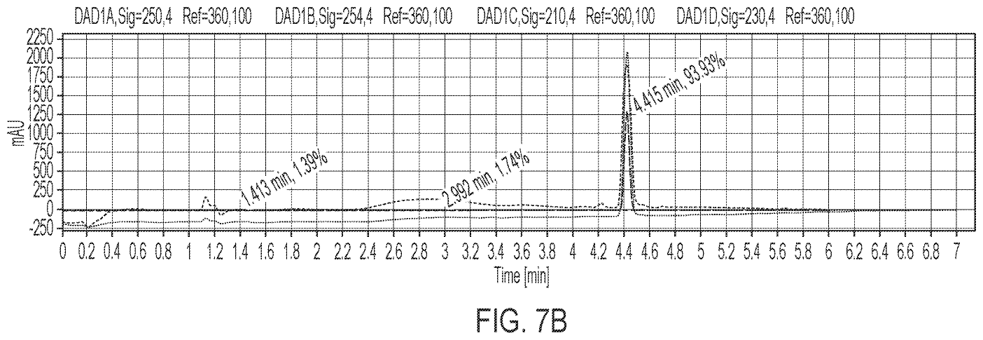

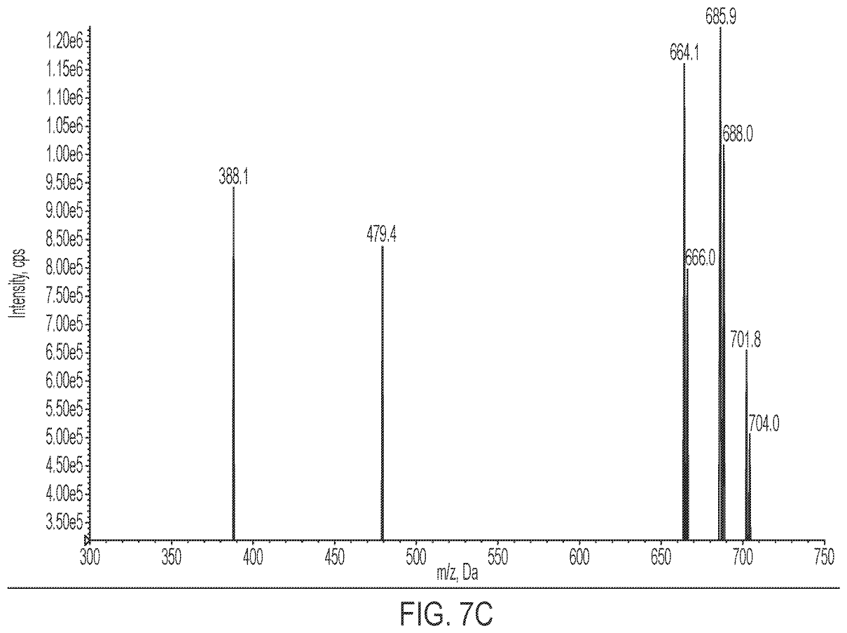

### DETAILED DESCRIPTION

### Definitions

It is to be understood that both the foregoing general description and the following detailed description are exemplary and explanatory only and are not restrictive of the embodiments, as claimed. Herein, the use of the singular includes the plural unless specifically stated otherwise. As used herein, the use of “or” means “and/or” unless stated otherwise. Furthermore, the use of the term “including” as well as other forms, such as “includes” and “included,” is not limiting. The section headings used herein are for organizational purposes only and are not to be construed as limiting the subject matter described.

Unless otherwise indicated, the following terms have the following meanings:

As used herein, the term “treating” a disorder encompasses ameliorating, mitigating and/or managing the disorder and/or conditions that may cause the disorder. The terms “treating” and “treatment” refer to a method of alleviating or abating a disease and/or its attendant symptoms. In accordance with the present disclosure, “treating” includes blocking, inhibiting, attenuating, protecting against, modulating, reversing the effects of, and reducing the occurrence of, e.g., the harmful effects of a disorder. As used herein, “inhibiting” encompasses preventing, reducing, and halting progression.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high-performance liquid chromatography (HPLC). Particularly, in certain embodiments, the compound is at least 85% pure, more preferably at least 90% pure, more preferably at least 95% pure, and most preferably at least 99% pure.

The term “administration” or “administering” includes routes of introducing the compound(s) to a subject to perform their intended function. Examples of routes of administration which can be used include injection (subcutaneously, intravenously, parenterally, intraperitoneally, intrathecally), topical, oral, inhalation, rectal, and transdermal.

The term “effective amount” includes an amount effective, at dosages and for periods of time necessary, to achieve the desired result. An effective amount of compound may vary according to factors such as the disease state, age, and weight of the subject, and the ability of the compound to elicit a desired response in the subject. Dosage regimens may be adjusted to provide the optimum therapeutic response. An effective amount is also one in which any non-tolerable or detrimental effects (e.g., side effects) of the compound are outweighed by the therapeutically beneficial effects.

The phrases “systemic administration,” “administered systemically,” “peripheral administration” and “administered peripherally” as used herein mean the administration of a compound(s), oligonucleotide(s), drug, or other material, such that it enters the patient's circulatory system and, thus, is subject to metabolism and other like processes.

The term “therapeutically effective amount” refers to the amount of the compound being administered sufficient to prevent development of or alleviate to some extent one or more of the symptoms of the condition or disorder being treated.

A therapeutically effective amount of compound (i.e., an effective dosage) may range from about 0.005 g/kg to about 200 mg/kg, preferably about 0.01 mg/kg to about 200 mg/kg, and more preferably about 0.015 mg/kg to about 30 mg/kg of body weight. In other embodiments, the therapeutically effect amount may range from about 1.0 pM to about 10 M. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a compound can include a single treatment or, preferably, can include a series of treatments. In one example, a subject is treated with a compound in the range of between about 0.005 g/kg to about 200 mg/kg of body weight, daily, weekly, monthly, quarterly, or yearly. In another example, a subject may be treated daily, weekly, monthly, quarterly, or yearly for several years in the setting of a chronic condition or illness. It will also be appreciated that the effective dosage of a compound used for treatment may increase or decrease over the course of a particular treatment.

The term “chiral” refers to molecules that have the property of non-superimposability of the mirror image partner, while the term “achiral” refers to molecules that are superimposable on their mirror image partner.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)-or (S)- or, as (D)- or (L)-for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)-and (L)-isomers may be prepared using chiral synthons or chiral reagents or resolved using conventional techniques. When the compounds described herein contain olefinic bonds or other centers of geometric asymmetry, and unless specified otherwise, it is intended that the compounds include both E and Z geometric isomers.

The term “tautomer,” as used herein, refers to one of two or more structural isomers which exist in equilibrium, and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure (i.e., the R and S configurations for each asymmetric center). Therefore, single stereochemical isomers as well as enantiomeric and diastereomeric mixtures of the present compounds are within the scope of the disclosure.

As used herein, “chirally enriched population” means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom. Chirally enriched populations of molecules having multiple chiral centers within each molecule may contain one or more stereorandom chiral centers. In certain embodiments, the molecules are modified oligonucleotides. In certain embodiments, the molecules are compounds comprising modified oligonucleotides.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

As used herein, “stereorandom chiral center” in the context of a population of molecules of identical molecular formula means a chiral center having a random stereochemical configuration. For example, in a population of molecules comprising a stereorandom chiral center, the number of molecules having the (S) configuration of the stereorandom chiral center may be but is not necessarily the same as the number of molecules having the (R) configuration of the stereorandom chiral center. The stereochemical configuration of a chiral center is considered random when it is the result of a synthetic method that is not designed to control the stereochemical configuration. In certain embodiments, a stereorandom chiral center is a stereorandom phosphorothioate internucleoside linkage.

The term “diastereomers” refers to stereoisomers with two or more centers of dissymmetry and whose molecules are not mirror images of one another.

The term “enantiomers” refers to two stereoisomers of a compound that are non-superimposable mirror images of one another. An equimolar mixture of two enantiomers is called a “racemic mixture” or a “racemate.”

The term “isomers” or “stereoisomers” refers to compounds that have identical chemical constitution but differ with regard to the arrangement of the atoms or groups in space.

The term “prodrug” is meant to indicate a compound that may be converted under physiological conditions or by solvolysis to a biologically active form of the compound (e.g., biologically active form of a nucleic acid) or analogue thereof as described herein. Thus, the term “prodrug” refers to a precursor of a biologically active compound (e.g., nucleic acid) or analogue thereof that is pharmaceutically acceptable. A prodrug may be inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. The term “prodrug” is also meant to include any covalently bonded carriers, which release the active compound in vivo when such prodrug is administered to a mammalian subject. Prodrugs of an active compound, as described herein, may be prepared by modifying functional groups present in the active compound in such a way that the modifications are cleaved, either in routine manipulation or in vivo, to the parent active compound. Prodrugs include compounds wherein a hydroxy, amino, or mercapto group is bonded to any group that, when the prodrug of the active compound is administered to a mammalian subject, cleaves to form a free hydroxy, free amino, or free mercapto group, respectively. Examples of suitable prodrugs include, but are not limited to, glutathione, acyloxy, thioacyloxy, 2-carboalkoxyethyl, disulfide, thiaminal, and enol ester derivatives of a phosphorus atom-modified nucleic acid. The term “pro-oligonucleotide” or “pronucleotide” or “nucleic acid prodrug” refers to an oligonucleotide which has been modified to be a prodrug of the oligonucleotide. Phosphonate and phosphate prodrugs can be found, for example, in Wiener et al., “Prodrugs or phosphonates and phosphates: crossing the membrane” Top. Curr. Chem. 2015, 360:115-160, the entirety of which is herein incorporated by reference. Prodrugs that are converted to active forms through other mechanisms in vivo are also included. In aspects, the compounds of the present disclosure are prodrugs of any of the formulae herein.

The term “prodrug” includes compounds with moieties that can be metabolized in vivo. Generally, the prodrugs are metabolized in vivo by esterases or by other mechanisms to active drugs. Examples of prodrugs and their uses are well known in the art (see, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branched or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Preferred prodrug moieties are propionoic acid esters and acyl esters. Prodrugs that are converted to active forms through other mechanisms in vivo are also included. In aspects, the compounds of the present disclosure are prodrugs of any of the formulae herein.

The term “subject” refers to animals such as mammals, including, but not limited to, primates (e.g., humans), cows, sheep, goats, horses, dogs, cats, rabbits, rats, mice, and the like. In certain embodiments, the subject is a human.

The terms “a,” “an,” and “the” refer to “one or more” when used in this application, including the claims. Thus, for example, reference to “a sample” includes a plurality of samples, unless the context clearly is to the contrary (e.g., a plurality of samples), and so forth.

Throughout this specification and the claims, the words “comprise,” “comprises,” and “comprising” are used in a non-exclusive sense, except where the context requires otherwise.

As used herein, the term “about,” when referring to a value, is meant to encompass variations of, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

As used herein, the term “alkyl,” by itself or as part of another substituent, means, unless otherwise stated, a straight-chained (i.e., unbranched) or branched carbon chain (or carbon), or combination thereof, which may be fully saturated, mono-, (e.g., alkene or alkenyl) or polyunsaturated (e.g., alkyne or alkynyl) and can include mono-, di-, and multivalent radicals, having the number of carbon atoms designated. For example, C₁-C₂₄ means 1 to 24 carbon atoms. A specified number of carbon atoms within this range includes, for example, C₁-C₂₀ alkyl (having 1-20 carbon atoms), C₁-C₁2 alkyl (having 1-12 carbon atoms) and C₁-C₄ alkyl (having 1-4 carbon atoms).

The term “alkenyl” refers to an unsaturated hydrocarbon chain that may be a straight chain or branched chain, containing 2 to 12 carbon atoms and at least one carbon-carbon double bond. Alkenyl groups may be optionally substituted with one or more substituents.

The term “alkynyl” refers to an unsaturated hydrocarbon chain that may be a straight chain or branched chain, containing the 2 to 12 carbon atoms and at least one carbon-carbon triple bond. Alkynyl groups may be optionally substituted with one or more substituents.

The term “lower alkyl” refers to a C₁-C₆ alkyl chain. Examples of alkyl groups include methyl, ethyl, n-propyl, isopropyl, tert-butyl, and n-pentyl. Alkyl groups may be optionally substituted with one or more substituents.

The term “haloalkyl” refers to an alkyl group that is substituted by one or more halo substituents. Examples of haloalkyl groups include fluoromethyl, difluoromethyl, trifluoromethyl, bromomethyl, chloromethyl, and 2,2,2-trifluoroethyl. The term “arylalkenyl” refers to an unsaturated hydrocarbon chain that may be a straight chain or branched chain, containing 2 to 12 carbon atoms and at least one carbon-carbon double bond wherein one or more of the sp² hybridized carbons of the alkenyl unit attaches to an aryl moiety. Alkenyl groups may be optionally substituted with one or more substituents.

The term “arylalkynyl” refers to an unsaturated hydrocarbon chain that may be a straight chain or branched chain, containing 2 to 12 carbon atoms and at least one carbon-carbon triple bond wherein one or more of the sp hybridized carbons of the alkynyl unit attaches to an aryl moiety. Alkynyl groups may be optionally substituted with one or more substituents.

The sp²—or sp-hybridized carbons of an alkenyl group and an alkynyl group, respectively, may optionally be the point of attachment of the alkenyl or alkynyl groups.

The term “alkoxy” refers to an —O-alkyl substituent.

As used herein, the terms “halogen,” hal,” or “halo” mean —F, —Cl, —Br or —I.

The term “alkylthio” refers to an —S-alkyl substituent.

The term “alkoxyalkyl” refers to an -alkyl-O-alkyl substituent.

The term “haloalkoxy” refers to an —O-alkyl that is substituted by one or more halo substituents. Examples of haloalkoxy groups include trifluoromethoxy, and 2,2,2-trifluoroethoxy.

The term “haloalkoxyalkyl” refers to an -alkyl-O-alkyl ‘where the alkyl’ is substituted by one or more halo substituents.

The term “haloalkylaminocarbonyl” refers to a —C(O)-amino-alkyl where the alkyl is substituted by one or more halo substituents.

The term “haloalkylthio” refers to an —S-alkyl that is substituted by one or more halo substituents. Examples of haloalkylthio groups include trifluoromethylthio, and 2,2,2-trifluoroethylthio.

The term “haloalkylcarbonyl” refers to an —C(O)-alkyl that is substituted by one or more halo substituents. An example of a haloalkylcarbonyl group includes trifluoroacetyl.

The term “cycloalkyl” refers to a hydrocarbon 3-8 membered monocyclic or 7-14 membered bicyclic ring system having at least one saturated ring or having at least one non-aromatic ring, wherein the non-aromatic ring may have some degree of unsaturation. Cycloalkyl groups may be optionally substituted with one or more substituents. In one embodiment, 0, 1, 2, 3, or 4 atoms of each ring of a cycloalkyl group may be substituted by a substituent. Representative examples of cycloalkyl group include cyclopropyl, cyclopentyl, cyclohexyl, cyclobutyl, cycloheptyl, cyclopentenyl, cyclopentadienyl, cyclohexenyl, cyclohexadienyl, and the like.

The term “cycloalkoxy” refers to an —O-cycloalkyl substituent.

The term “cycloalkoxyalkyl” refers to an -alkyl-O-cycloalkyl substituent.

The term “cycloalkylalkoxy” refers to an —O-alkyl-cycloalkyl substituent.

The term “cycloalkylaminocarbonyl” refers to an —C(O)—NH-cycloalkyl substituent.

The term “aryl” refers to a hydrocarbon monocyclic, bicyclic, or tricyclic aromatic ring system. Aryl groups may be optionally substituted with one or more substituents. In one embodiment, 0, 1, 2, 3, 4, 5 or 6 atoms of each ring of an aryl group may be substituted by a substituent. Examples of aryl groups include phenyl, naphthyl, anthracenyl, fluorenyl, indenyl, azulenyl, and the like.

The term “aryloxy” refers to an —O-aryl substituent.

The term “arylalkoxy” refers to an —O-alkyl-aryl substituent.

The term “arylalkylthio” refers to an —S-alkyl-aryl substituent.

The term “arylthioalkyl” refers to an -alkyl-S -aryl substituent.

The term “arylalkylaminocarbonyl” refers to a —C(O)-amino-alkyl-aryl substituent.

The term “arylalkylsulfonyl” refers to an —S(O)₂-alkyl-aryl substituent.

The term “arylalkylsulfinyl” refers to an —S(O)-alkyl-aryl substituent.

The term “aryloxyalkyl” refers to an -alkyl-O-aryl substituent.

The term “alkylaryl” refers to an -aryl-alkyl substituent.

The term “arylalkyl” refers to an -alkyl-aryl substituent.

The term “heteroalkyl” by itself or in combination with another term, means, unless otherwise stated, a stable straight or branched chain, or combinations thereof, including at least one carbon atom and at least one heteroatom (e.g., O, N, P, Si, and/or S), and wherein the nitrogen and sulfur atoms may optionally be oxidized, and the nitrogen heteroatom may optionally be quaternized. The heteroatom(s) (e.g., O, N, P, Si, and/or S) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, CH₂—CH₂—NH—CH₃, CH₂—CH₂—N(CH₃)—CH₃, CH₂—S—CH₂—CH₃, CH₂—CH₂, S(O)—CH₃, CH₂—CH₂—S(O)₂—CH₃, CH═CHO—CH₃, Si(CH₃)₃, CH₂ CH═N OCH₃, CH═CH N(CH₃)—CH₃, O CH₃, 0 CH₂—CH₃, and CN. Up to two or three heteroatoms may be consecutive, such as, for example, CH₂—NH—OCH₃ and CH₂—O—Si(CH₃)₃. A heteroalkyl moiety may include one heteroatom (e.g., O, N, S, Si, B, or P). A heteroalkyl moiety may include two optionally different heteroatoms (e.g., O, N, S, Si, B, and/or P). A heteroalkyl moiety may include three optionally different heteroatoms (e.g., O, N, S, Si, B, and/or P). A heteroalkyl moiety may include four optionally different heteroatoms (e.g., O, N, S, Si, B, and/or P). A heteroalkyl moiety may include five optionally different heteroatoms (e.g., O, N, S, Si, B, and/or P). A heteroalkyl moiety may include up to 8 or more optionally different heteroatoms (e.g., O, N, S, Si, B, and/or P).

Similarly, the term “heteroalkylene,” by itself or as part of another substituent, means, unless otherwise stated, a divalent radical derived from heteroalkyl, as exemplified, but not limited by, CH₂—CH₂—S—CH₂—CH₂— and CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula C(O)₂R′ represents both C(O)₂R‘ and R’C(O)₂-. As described above, heteroalkyl groups, as used herein, include those groups that are attached to the remainder of the molecule through a heteroatom, such as C(O)R′, C(O)NR′, NR′R¹, OR′, SR′, and/or SO₂R′. Where “heteroalkyl” is recited, followed by recitations of specific heteroalkyl groups, such as NR′R″ or the like, it will be understood that the terms heteroalkyl and NR′R″ are not redundant or mutually exclusive. Rather, the specific heteroalkyl groups are recited to add clarity. Thus, the term “heteroalkyl” should not be interpreted herein as excluding specific heteroalkyl groups, such as NR′R″ or the like.

The term “alkylene,” by itself or as part of another substituent, means, unless otherwise stated, a divalent radical derived from an alkyl, as exemplified, but not limited by, CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. “Cycloalkyl” is also meant to refer to bicyclic and polycyclic hydrocarbon rings such as, for example, bicyclo[2.2.1]heptane, bicyclo[2.2.2]octane, etc.

The term “heteroaryl” refers to an aromatic 5-8 membered monocyclic, 8-12 membered bicyclic, or 11-14 membered tricyclic ring system having 1-4 ring heteroatoms if monocyclic, 1-6 heteroatoms if bicyclic, or 1-9 heteroatoms if tricyclic, said heteroatoms selected from O, N, or S, and the remainder ring atoms being carbon (with appropriate hydrogen atoms unless otherwise indicated). Heteroaryl groups may be optionally substituted with one or more substituents. In one embodiment, 0, 1, 2, 3, or 4 atoms of each ring of a heteroaryl group may be substituted by a substituent. Heteroaryl groups may be fully unsaturated, or they may be partially unsaturated and partially saturated. Examples of heteroaryl groups include pyridyl, furanyl, thienyl, pyrrolyl, oxazolyl, oxadiazolyl, imidazolyl thiazolyl, isoxazolyl, quinolinyl, pyrazolyl, isothiazolyl, pyridazinyl, pyrimidinyl, pyrazinyl, triazinyl, isoquinolinyl, indazolyl, and the like.

The term “heteroarylalkyl” refers to an -alkyl-heteroaryl substituent.

The term “heteroaryloxy” refers to an —O-heteroaryl substituent.

The term “heteroarylalkoxy” refers to an —O-alkyl-heteroaryl substituent.

The term “heteroaryloxyalkyl” refers to an -alkyl-O-heteroaryl substituent.

The term “nitrogen-containing heteroaryl” refers to a heteroaryl group having 1-4 ring nitrogen heteroatoms if monocyclic, 1-6 ring nitrogen heteroatoms if bicyclic, or 1-9 ring nitrogen heteroatoms if tricyclic.

The term “heterocycloalkyl” refers to a nonaromatic 3-8 membered monocyclic, 7-12 membered bicyclic, or 10-14 membered tricyclic ring system comprising 1-3 heteroatoms if monocyclic, 1-6 heteroatoms if bicyclic, or 1-9 heteroatoms if tricyclic, said heteroatoms selected from O, N, S, B, P or Si, wherein the nonaromatic ring system is completely saturated. Heterocycloalkyl groups may be optionally substituted with one or more substituents. In one embodiment, 0, 1, 2, 3, or 4 atoms of each ring of a heterocycloalkyl group may be substituted by a substituent. Representative heterocycloalkyl groups include piperidinyl, piperazinyl, tetrahydropyranyl, morpholinyl, thiomorpholinyl, 1,3-dioxolane, tetrahydrofuranyl, tetrahydrothienyl, thiirenyl, and the like.

The term “heterocycloalkylalkyl” refers to an -alkyl-heterocycloalkyl substituent.

The term “alkylamino” refers to an amino substituent which is further substituted with one or two alkyl groups. The term “aminoalkyl” refers to an alkyl substituent which is further substituted with one or more amino groups. The term “hydroxyalkyl” or “hydroxylalkyl” refers to an alkyl substituent which is further substituted with one or more hydroxyl groups. The alkyl or aryl portion of alkylamino, aminoalkyl, mercaptoalkyl, hydroxyalkyl, mercaptoalkoxy, sulfonylalkyl, sulfonylaryl, alkylcarbonyl, and alkylcarbonylalkyl may be optionally substituted with one or more substituents.

The symbol “-“denotes the point of attachment of a chemical moiety to the remainder of a molecule or chemical formula.

The term “nucleobase” refers to nitrogen-containing biological compounds that form nucleosides. They include purine bases and pyrimidine bases. Five nucleobases-adenine (A), cytosine (C), guanine (G), thymine (T), and uracil (U)-are referred to as primary or canonical nucleobases. When a nucleobase is listed in a formula definition, it refers to that moiety covalently bonded to the recited formula.

The term “modified nucleobase” refers to derivatives of a nucleobase. Examples of modified nucleobases include, but are not limited to, xanthine, hypoxanthine,7-methylguanine, 5,6-dihydrouracil, 5-methylcytosine, 5-hydroxymethylcytosine, purine, 2,6-diaminopurine, and 6,8-diaminopurine. When a modified nucleobase is listed in a formula definition, it refers to that moiety covalently bonded to the recited formula.

The term “substituent” and “substituent group” means an atom or group that replaces the atom or group of a named parent compound. For example, a substituent of a modified nucleoside is an atom or group that differs from the atom or group found in a naturally occurring nucleoside (e.g., a modified 2′-substituent is any atom or group at the 2′-position of a nucleoside other than H or OH). Substituent groups can be protected or unprotected. Substituents may also be further substituted with other substituent groups and may be attached directly or via a linking group such as an alkyl or hydrocarbyl group to the parent compound. Similarly, as used herein, “substituent” in reference to a chemical functional group means an atom or group of atoms that differs from the atom or group of atoms normally present in the named functional group. In certain embodiments, substituents on any group (such as, for example, alkyl, alkenyl, alkynyl, aryl, aralkyl, heteroaryl, heteroaralkyl, cycloalkyl, heterocycloalkyl) can be at any atom of that group, wherein any group that can be substituted (such as, for example, alkyl, alkenyl, alkynyl, aryl, aralkyl, heteroaryl, heteroaralkyl, cycloalkyl, heterocycloalkyl) can be optionally substituted with one or more substituents (which may be the same or different), each replacing a hydrogen atom. Examples of suitable substituents include, but are not limited to alkyl, alkenyl, alkynyl, cycloalkyl, heterocycloalkyl, aralkyl, heteroaralkyl, aryl, heteroaryl, halogen, haloalkyl, cyano, nitro, alkoxy, aryloxy, hydroxyl, hydroxylalkyl, oxo (i.e., carbonyl), carboxyl, formyl, alkylcarbonyl, alkylcarbonylalkyl, alkoxycarbonyl, alkylcarbonyloxy, aryloxycarbonyl, heteroaryloxy, heteroaryloxycarbonyl, thio, mercapto, mercaptoalkyl, arylsulfonyl, amino, aminoalkyl, dialkylamino, alkylcarbonylamino, alkylaminocarbonyl, alkoxycarbonylamino, alkylamino, arylamino, diarylamino, alkylcarbonyl, or arylamino-substituted aryl; arylalkylamino, aralkylaminocarbonyl, amido, alkylaminosulfonyl, arylaminosulfonyl, dialkylaminosulfonyl, alkylsulfonylamino, arylsulfonylamino, imino, carboxamido, carbamido, carbamyl, thioureido, thiocyanato, sulfoamido, sulfonylalkyl, sulfonylaryl, mercaptoalkoxy, N-hydroxyamidinyl, or N′-aryl, N″-hydroxyamidinyl. In certain embodiments, substituents on any group include alkyl, alkenyl, alkynyl, cycloalkyl, heterocycloalkyl, aralkyl, heteroaralkyl, aryl, heteroaryl, halogen, haloalkyl, cyano, nitro, alkoxy, aryloxy, hydroxyl, hydroxylalkyl, oxo (i.e., carbonyl), carboxyl, formyl, alkylcarbonyl, alkylcarbonylalkyl, alkoxycarbonyl, alkylcarbonyloxy, thiocarbonyl, thio, mercapto, mercaptoalkyl, arylsulfonyl, amino, aminoalkyl, dialkylamino, alkylcarbonylamino, alkylaminocarbonyl, alkoxycarbonylamino, alkylamino, arylamino, diarylamino, alkylcarbonyl, or arylamino-substituted aryl; arylalkylamino, aralkylaminocarbonyl, or amido. In certain embodiments, substituents on any group include alkyl, halogen, haloalkyl, cyano, nitro, alkoxy, hydroxyl, hydroxylalkyl, carboxyl, formyl, alkylcarbonyl, alkoxycarbonyl, alkylcarbonyloxy, thio, mercapto, mercaptoalkyl, amino, aminoalkyl, dialkylamino, alkylcarbonylamino, alkylaminocarbonyl, or alkylamino.

The term “protecting group” or “protecting moiety” refers to a substituent that is commonly employed to block or protect a particular functionality while reacting other functional groups on the compound, a derivative thereof, or a conjugate thereof, and includes a nitrogen protecting group when attached to a nitrogen atom, or an oxygen protecting group when attached to an oxygen atom. Nitrogen and oxygen protecting groups are well known in the art and include those described in detail in Protecting Groups in Organic Synthesis, T. W. Greene and P. G. M. Wuts, 3rd edition, John Wiley & Sons, 1999, incorporated herein by reference.

In certain embodiments, the substituent present on a nitrogen atom is a nitrogen protecting group (also referred to as an amino protecting group). Nitrogen protecting groups include, but are not limited to, —OH, —OR^{aa}, —N(R^{cc})₂, —C(═O)R¹, —C(═O)N(R^{ee}), —CO₂R^{aa}, —SO₂R^{aa}, —C(═NR^{cc})R¹, —C(═NR^{cc})OR^{aa}, —C(═NR^{cc})N(R^{cc})₂, —SO₂N(R^{cc} )₂, —SO₂R¹, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 R^{dd} groups, and wherein each R^{aa}, R^{bb}, and R^{cc} is independently alkyl, cycloalkyl, aryl, or heteroaryl, each of which may be optionally substituted with 1-3 independent R^{dd}, and each R^{dd} is independently alkyl, alkenyl, alkynyl, cycloalkyl, heterocycloalkyl, aralkyl, heteroaralkyl, aryl, heteroaryl, halogen, haloalkyl, cyano, nitro, alkoxy, aryloxy, hydroxyl, hydroxylalkyl, oxo (i.e., carbonyl), carboxyl, formyl, alkylcarbonyl, alkylcarbonylalkyl, alkoxycarbonyl, alkylcarbonyloxy, aryloxycarbonyl, heteroaryloxy, heteroaryloxycarbonyl, thio, mercapto, mercaptoalkyl, arylsulfonyl, amino, aminoalkyl, dialkylamino, alkylcarbonylamino, alkylaminocarbonyl, alkoxycarbonylamino, alkylamino, arylamino, diarylamino, alkylcarbonyl, or arylamino-substituted aryl; arylalkylamino, aralkylaminocarbonyl, amido, alkylaminosulfonyl, arylaminosulfonyl, dialkylaminosulfonyl, alkylsulfonylamino, arylsulfonylamino, imino, carbamido, carbamyl, thioureido, thiocyanato, sulfoamido, sulfonylalkyl, sulfonylaryl, or mercaptoalkoxy. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Amide nitrogen protecting groups (e.g., —C(═O)R^{a}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine, o-nitrobenzamide, and o-(benzoyloxymethyl)benzamide.

Carbamate nitrogen protecting groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Sulfonamide nitrogen protecting groups (e.g., —S(═O)₂R′) include, but are not limited to, p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), 0-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N-(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to as a hydroxyl protecting group). Oxygen protecting groups include, but are not limited to, —R^{aa}, —N(R^{bb})₂, —C(═O)SR^{aa}, —C(═O)R^{aa}, —CO₂R′, —C(═O)N(R^{bb})₂, —C(═NR^{bb})R^{aa}, —C(═NR^{bb})OR—, —C(═NR^{bb})N(R^{bb})₂, —S(═O)R^{aa}, —SO₂R, —Si(R^{aa})₃, —P(R^{cc})₂, —P(R^{aa})₃, —P(═O)₂R¹, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), t-butyl carbonate (BOC), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a thiol protecting group). Sulfur protecting groups include, but are not limited to, —R^{aa}, —N(R^{bb})₂, —C(═O)SR^{aa}, —C(═O)R^{aa}—, —CO₂R′, —C(═O)N(R^{bb})₂, —C(═NR^{bb})R-, —C(=NR^{bb})OR—, —C(═NR^{bb})N(R^{bb})₂, —S(═O)R^{aa}, —SO₂R′, —Si(R^{aa})₃, —P(R^{cc})₂, —P(R^{cc})₃, —P(═O)₂R¹, —P(═O)(R^{cc})₂, —P(═O)(OR^{cc})₂, —P(═O)₂N(R^{bb})₂, and —P(═O)(NR^{bb})₂, wherein R¹, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

The term “antisense oligonucleotide” or “antisense strand” means an oligonucleotide which includes a region that is complementary to a target nucleic acid.

The term “composition” or “pharmaceutical composition” means a mixture of substances suitable for administering to a subject. For example, a composition may comprise one or more compounds or salt thereof and a sterile aqueous solution.

The term “nucleic acid” refers to molecules composed of linked monomeric nucleotides or nucleosides. A nucleic acid includes, but is not limited to, ribonucleic acids (RNA), deoxyribonucleic acids (DNA), single-stranded nucleic acids, and double-stranded nucleic acids.

The term “nucleobase sequence” means the order of contiguous nucleobases in a nucleic acid or oligonucleotide independent of any sugar or internucleoside linkage.

The term “nucleoside” means a compound comprising a nucleobase and a sugar moiety. The nucleobase and sugar moiety are each, independently, unmodified or modified. “Modified nucleoside” means a nucleoside comprising a modified nucleobase and/or a modified sugar moiety. Modified nucleosides include abasic nucleosides, which lack a nucleobase.

The term “oligomeric compound” means a polymer of linked subunits. With reference to a protein, peptide, polypeptide, or antibody, “subunit” refers to an amino acid or peptide bond. With reference to an oligonucleotide, “subunit” refers to a nucleotide, nucleoside, nucleobase, or sugar, or a modified nucleotide, nucleoside, nucleobase, or sugar as provided herein.

The term “oligonucleotide” means a polymer of linked nucleosides (e.g., polynucleotide, nucleic acid, polymer of nucleotides), each of which can be modified or unmodified, independent from one another. Without limitation, an oligonucleotide may be comprised of ribonucleic acids (e.g., comprised of ribonucleosides), deoxyribonucleic acids (e.g., comprised of deoxyribonucleosides), modified nucleic acids (e.g., comprised of modified nucleobases, sugars, and/or phosphate groups), or a combination thereof. Examples of oligonucleotide compounds include single-stranded and double-stranded compounds, such as oligonucleotides, antisense oligonucleotides, interfering RNA compounds (RNAi compounds), microRNA (miRNA) targeting oligonucleotides, miRNA mimics, occupancy-based compounds (e.g., mRNA processing or translation blocking compounds and splicing compounds) and editing compounds (e.g., ADAR recruiting molecules, ADAR targeting molecules, single-stranded guide nucleic acids, or a combination thereof). RNAi compounds include double-stranded compounds (e.g., short-interfering RNA (siRNA) and double-stranded RNA (dsRNA)) and single-stranded compounds (e.g., single-stranded siRNA (ssRNA), single-stranded RNAi (ssRNAi), short hairpin RNA (shRNA), and microRNA mimics) which work at least in part through the RNA-induced silencing complex (RISC) pathway resulting in sequence specific degradation and/or sequestration of a target nucleic acid through a process known as RNA interference (RNAi). The term “RNAi compound” is meant to be equivalent to other terms used to describe nucleic acid compounds that are capable of mediating sequence-specific RNA interference, for example, interfering RNA (iRNA), iRNA agent, RNAi agent, small interfering RNA, short interfering RNA, short interfering oligonucleotide, short interfering nucleic acid, short interfering modified oligonucleotide, chemically modified siRNA, and others. Additionally, the term “RNAi” is meant to be equivalent to other terms used to describe sequence-specific RNA interference.

The term “target nucleic acid,” “target RNA,” and “nucleic acid target” all mean a nucleic acid capable of being targeted by compounds described herein.

The term “therapeutic compound” includes any pharmaceutical agent or compound that provides a therapeutic benefit to a subject. Therapeutic compounds include nucleic acids, oligomeric compounds, oligonucleotides, proteins, peptides, antibodies, small molecules, and other such agents.

“Target region” means a portion of a target nucleic acid to which one or more compounds is targeted.

“Targeting moiety” means a conjugate group that provides an enhanced affinity for a selected target, e.g., molecule, cell or cell type, compartment, e.g., a cellular or organ compartment, tissue, organ, or region of the body, as, e.g., compared to a compound absent such a moiety.

“Terminal group” means a chemical group or group of atoms that is covalently linked to a terminus of an oligonucleotide.

“Derivative” means a molecule or compound described herein that has been transformed by one chemical reaction.

The term “ligand” refers to a substance that binds to or otherwise interacts with a protein, nucleic acid, or other biological molecule. In some embodiments, a ligand is a small molecule. In some embodiments, a ligand binds to a protein (e.g., a receptor). In certain embodiments, a ligand binds to an α₄β_{1/7} integrin receptor.

The term α₄β_{1/7} in integrin receptor” refers to heterodimeric integrin receptors formed by association of integrin alpha 4 and integrin beta 1 (i.e., the α₄β_{1/7} integrin receptor) and integrin alpha 4 and integrin beta 7 (i.e., the α₄β_{1/7} integrin receptor). In certain embodiments, the α₄β_{1/7} integrin receptor ligand has a higher binding affinity for α₄β_{1/7} integrin receptor than α₄β_{1/7} integrin receptor. In certain embodiments, the α₄β₁₇ integrin receptor ligand has a higher binding affinity for α₄β_{1/7} integrin receptor than α₄β_{1/7} integrin receptor.

The term “sense oligonucleotide” or “sense strand” means the strand of a double-stranded compound that includes a region that is substantially complementary to a region of the antisense strand of the double-stranded compound.

The terms “microRNA” and “miRNA,” as may be used interchangeably herein, refer to short (e.g., about 20 to about 24 nucleotides in length) non-coding ribonucleic acids (RNAs) that are involved in post-transcriptional regulation of gene expression in multicellular organisms by affecting both the stability and translation of mRNAs. miRNAs are transcribed by RNA polymerase II as part of capped and polyadenylated primary transcripts (pri-miRNAs) that can be either protein-coding or non-coding. The primary transcript is cleaved by the Drosha ribonuclease III enzyme to produce a stem-loop precursor miRNA (pre-miRNA) approximately 70 nucleotides in length, which is further processed in the RNAi pathway. As part of this pathway, the pre-miRNA is cleaved by the cytoplasmic Dicer ribonuclease to generate the mature miRNA and antisense miRNA star (miRNA*) products. The mature miRNA is incorporated into an RNA-induced silencing complex (RISC), which recognizes target mRNAs through imperfect base pairing (i.e., partial complementarity) with the miRNA and most commonly results in translational inhibition or destabilization of the target mRNA. This mechanism is most often seen through the binding of the miRNA on the 3′ untranslated region (UTR) of the target mRNA, which can decrease gene expression by either inhibiting translation (for example, by blocking the access of ribosomes for translation) or directly causing degradation of the transcript. The term (i.e., miRNA) may be used herein to refer to any form of the subject miRNA (e.g., precursor, primary, and/or mature miRNA).

The terms “small interfering RNA” “short interfering RNA” and “siRNA,” as may be used interchangeably herein, refer to RNA molecules that present as non-coding double-stranded RNA (dsRNA) molecules of about 20 to about 24 nucleotides in length and are useful in RNA interference (RNAi). siRNA are often found with phosphorylated 5′ ends and hydroxylated 3′ ends, which 3′ ends typically have a 2-nucleotide overhang beyond the 5′ end of the anti-parallel strand (e.g., complementary strand of the dsRNA molecule). siRNA can interfere with the expression of specific genes through binding of target sequences (e.g., target nucleic acid sequences) to which they are complementary and promoting (e.g., facilitating, triggering, initiating) degradation of the mRNA, thereby preventing (e.g., inhibiting, silencing, interfering with) translation. After integration and separation into the RISC complex, siRNAs base-pair (e.g., full complementarity) to their target mRNA and cleave it, thereby preventing it from being used as a translation template. As discussed herein above, also part of the RNAi pathway, a miRNA-loaded RISC complex scans cytoplasmic mRNAs for potential complementarity (e.g., partial complementarity).

The term “ADAR recruiting molecule,” as may be used herein, refers to a nucleic acid that is configured to increase the concentration of Adenosine Deaminase Acting on Ribonucleic Acid (ADAR) enzyme in a locality around the nucleic acid. In some embodiments, an increased concentration is relative to the concentration in a given locality absent the ADAR recruiting molecule. In some embodiments, an ADAR recruiting molecule comprises a double-stranded RNA duplex.

The term “ADAR targeting molecule,” as may be used herein, refers to a nucleic acid that is configured to direct an ADAR molecule to a desirable location (e.g., locality). As used herein, the term “direct” refers to increasing the concentration of ADAR in the desirable location as compared to the concentration absent the ADAR targeting molecule. In some embodiments, the ADAR targeting molecule can be configured to control the desirable location by altering the sequence and/or properties of the nucleic acid (e.g., by modifications to the nucleobase, sugar, internucleoside linkage, or other component). In some embodiments, an ADAR targeting molecule comprises an ADAR recruiting molecule and a single-stranded guide nucleic acid. In some embodiments, an ADAR targeting molecule comprises a double-stranded RNA duplex and a single-stranded guide nucleic acid.

The term “single-stranded guide nucleic acid” or “guide RNA” as may be used herein, refers to a nucleic acid of a single strand, which comprises a specific sequence that is at least partially complementary to a target sequence. In some embodiments, the target sequence is at, adjacent to, or in proximity to, a locality where it is desirable to modulate ADAR concentration. In some embodiments, the level of complementarity is sufficient to facilitate binding (e.g., annealing) of the single-stranded guide nucleic acid to the target sequence.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

The term “isotopic variant” refers to a therapeutic agent (e.g., a compound and/or modified oligonucleotide disclosed herein) that contains an unnatural proportion of an isotope at one or more of the atoms that constitute such a therapeutic agent. In certain embodiments, an “isotopic variant” of a therapeutic agent contains unnatural proportions of one or more isotopes, including, but not limited to, hydrogen (H), deuterium (²H), tritium (3H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (140), oxygen-15 (150), oxygen-16 (160), oxygen-17 (17), oxygen-18 (180), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵C₁), chlorine-36 (³⁶C₁), chlorine-37 (³⁷C₁), bromine-79 (⁷⁹Br), bromine-81 (⁸1Br), iodine 123 (¹²³I), iodine-125 (¹²5I), iodine-127 (¹²⁷1) iodine-129 (¹²91) and iodine-131 (¹³¹1) In certain embodiments, an “isotopic variant” of a therapeutic agent contains unnatural proportions of one or more isotopes, including, but not limited to, hydrogen (H), deuterium (²H), tritium (3H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (140), oxygen-15 (150), oxygen-16 (160), oxygen-17 (17), oxygen-18 (¹80), fluorine-17 (¹⁷F), fluorine-18 (¹⁸F), phosphorus-31 (³¹P), phosphorus-32 (³²p), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵C₁), chlorine-36 (³⁶C₁), chlorine-37 (³⁷C₁), bromine-79 (⁷⁹Br), bromine-81 (⁸¹Br), iodine 123 (¹²3I), iodine-125 (¹²5I), iodine-127 (¹²7I), iodine-129 (¹²⁹1) and iodine-131 (1311)

It will be understood that, in a therapeutic agent (e.g., a compound and/or modified oligonucleotide disclosed herein), any hydrogen can be ²H, for example, or any carbon can be ¹³C, for example, or any nitrogen can be ¹⁵N, for example, or any oxygen can be ¹⁸O, for example, where feasible according to the judgment of one of skill. In certain embodiments, an “isotopic variant” of a therapeutic agent contains unnatural proportions of deuterium (D).

“Modified oligonucleotide” means an oligonucleotide, wherein at least one sugar, nucleobase, or internucleoside linkage is modified.

“Nucleobase sequence” means the order of contiguous nucleobases in a nucleic acid or oligonucleotide independent of any sugar or internucleoside linkage.

The term “oligomeric duplex” means a duplex formed by two oligomeric compounds having complementary nucleobase sequences. Each oligomeric compound of an oligomeric duplex may be referred to as a “duplexed oligomeric compound.” The oligonucleotides of each oligomeric compound of an oligomeric duplex may include non-complementary overhanging nucleosides. In some embodiments, the terms “duplexed oligomeric compound” and “modified oligonucleotide” are used interchangeably. In other embodiments, the terms “oligomeric duplex” and “compound” are used interchangeably.

“Phosphorothioate linkage” means a modified phosphate linkage in which one of the non-bridging oxygen atoms is replaced with a sulfur atom.

The terms “RNA interference compound,” “RNAi compound,” and/or “iRNA agent” mean a compound that acts, at least in part, through an RNA-induced silencing complex (RISC) pathway or Ago2, but not through RNase H, to modulate a target nucleic acid and/or protein encoded by a target nucleic acid. RNAi compounds include, but are not limited to double-stranded siRNA, single-stranded siRNA, and microRNA, including microRNA mimics.

### Certain Embodiments

In certain embodiment, a compound comprises an α₄β_{1/7} ligand and one or more linker moieties. In certain embodiments, the compound is selected from any of the formulae provided herein. In certain embodiments, the one or more linker moieties (L₁, L₂, L₃, L₄, L_{1A}, L_{2A}, L_{3A}, L_{4A}, etc.) links the α₄β_{1/7} ligand to a therapeutic, prophylactic, or diagnostic agent. In certain embodiments, the compound further comprises one or more therapeutic, prophylactic, or diagnostic agents. In certain embodiments, a therapeutic, prophylactic, or diagnostic agent is a small molecule, or an oligomeric compound. In certain embodiments, the oligomeric compound comprises a protein, a peptide, an antibody, an oligonucleotide, or a combination thereof.

In certain embodiments, an oligomeric compound is any of those described herein. In certain embodiments, the oligomeric compound is about 10-50 subunits in length. In certain embodiments the oligomeric compound is an oligonucleotide. In certain embodiments, an oligonucleotide is any of those described herein. In certain embodiments, the oligonucleotide is 8 to 80 linked nucleosides in length, 12-50 linked nucleosides in length, 12-30 linked nucleosides in length, or 15-30 linked nucleosides in length.

In certain embodiments, the oligonucleotide is a modified oligonucleotide comprising at least one modified internucleoside linkage, at least one modified sugar, or at least one modified nucleobase.

In certain embodiments, the oligonucleotide is single-stranded. In certain embodiments, the oligonucleotide is double-stranded. In certain embodiments, the oligonucleotide is double-stranded over a portion of its length. In certain embodiments, the oligonucleotide comprises ribonucleic acids (e.g., comprised of ribonucleosides), deoxyribonucleic acids (e.g., comprised of deoxyribonucleosides), or a combination thereof. In certain embodiments, the oligonucleotide is a small interfering RNA (siRNA), a microRNA (miRNA) antagonist, a miRNA mimic, an ADAR recruiting molecule, an ADAR targeting molecule, a guide RNA, an antisense oligonucleotide, a short hairpin RNA (shRNA), or combinations thereof.

In some embodiments, a linker is a bond. In some embodiments, a linker is an optionally substituted PEG linker. In some embodiments, a linker is two, three, four, five, six, seven, eight, nine, or ten PEG units in length. In certain embodiments, a linker comprises the structure

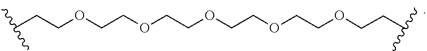 [SMILES: *CCOCCOCCOCCOCCOCC*]

In some embodiments, a linker is an optionally substituted heteroaryl linker. In some embodiments, a linker is an optionally substituted partially unsaturated heteroaryl linker. In some embodiments, a linker comprises the structure

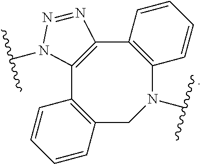 [SMILES: *N1Cc2ccccc2-c2c(nnn2*)-c2ccccc21]

In some embodiments, a linker is an optionally substituted heteroalkyl linker. In some embodiments, a linker is substituted with one or more ═O substituents. In some embodiments, a linker comprises two substituents joined together to form an optionally substituted carbocyclyl ring. In certain embodiments, a linker comprises the structure

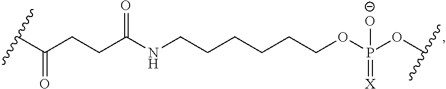

wherein X is O or S. In certain embodiments, a linker comprises the structure

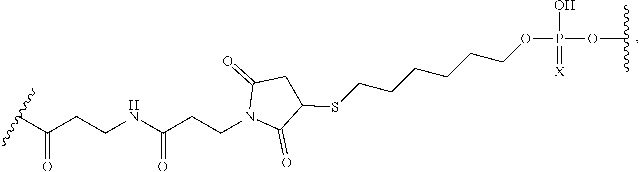

wherein X is O or S.

In some embodiments, a linker comprises the structure

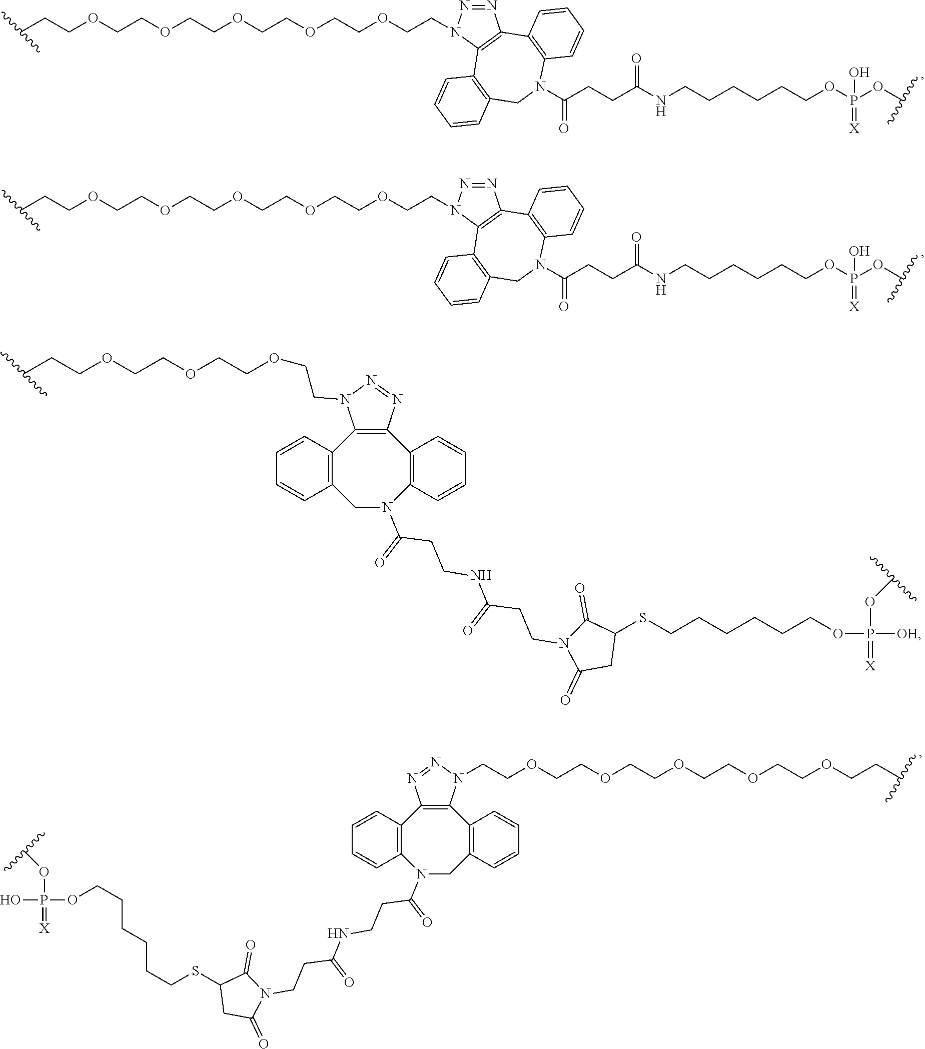

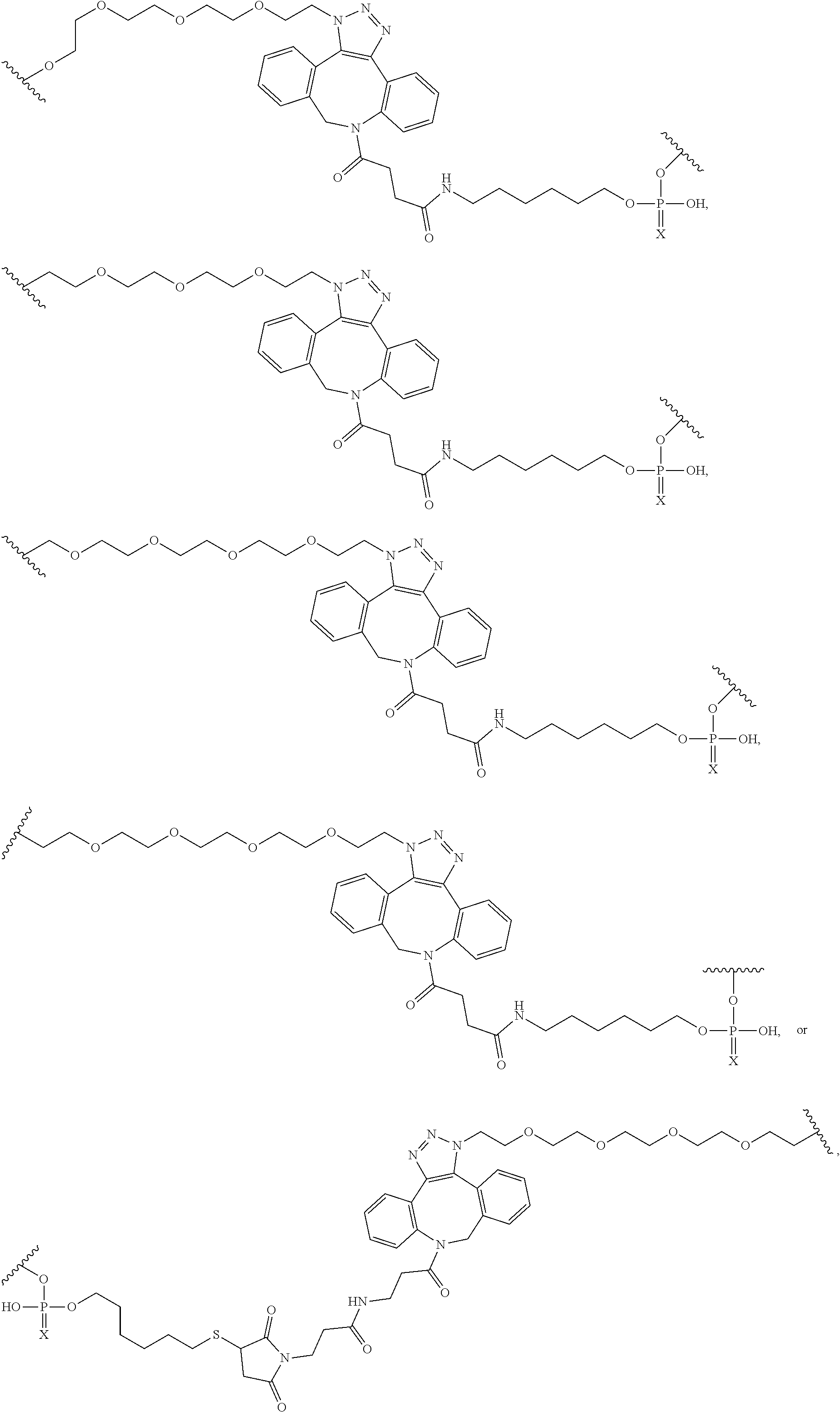

In certain embodiments, a compound comprises or consists of one of the structure:

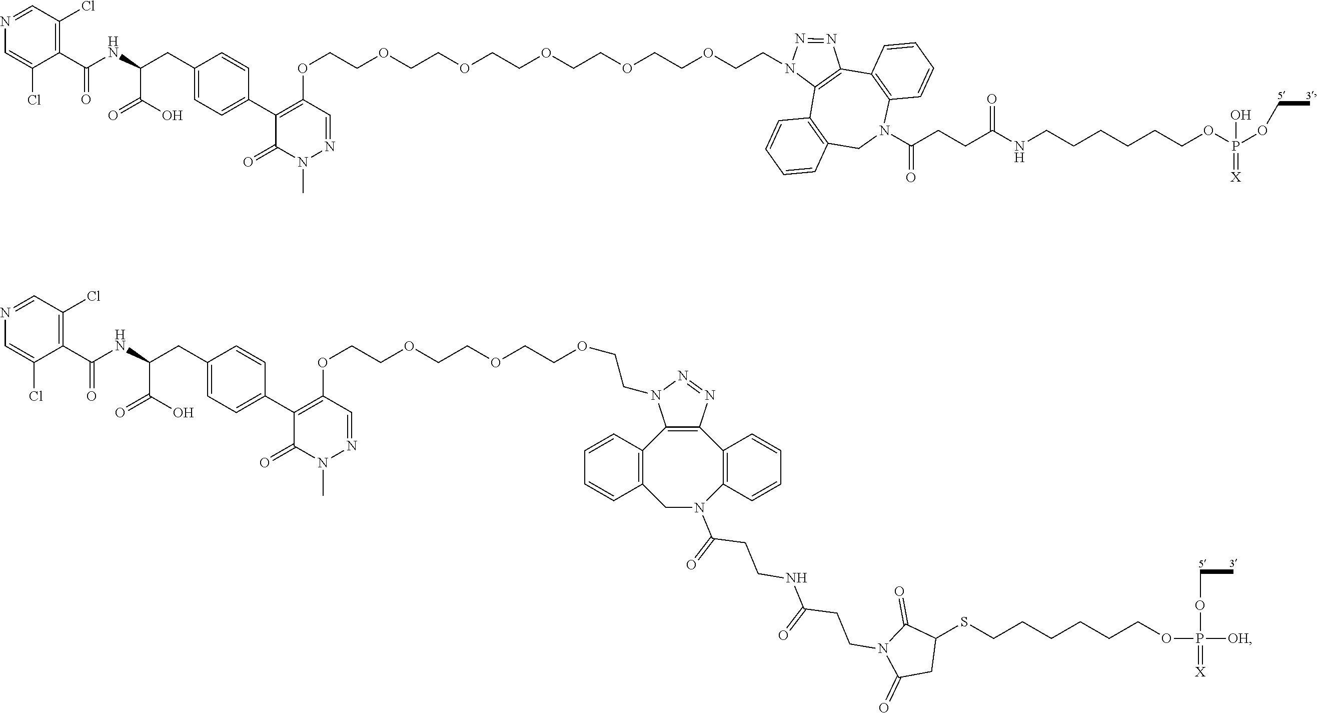

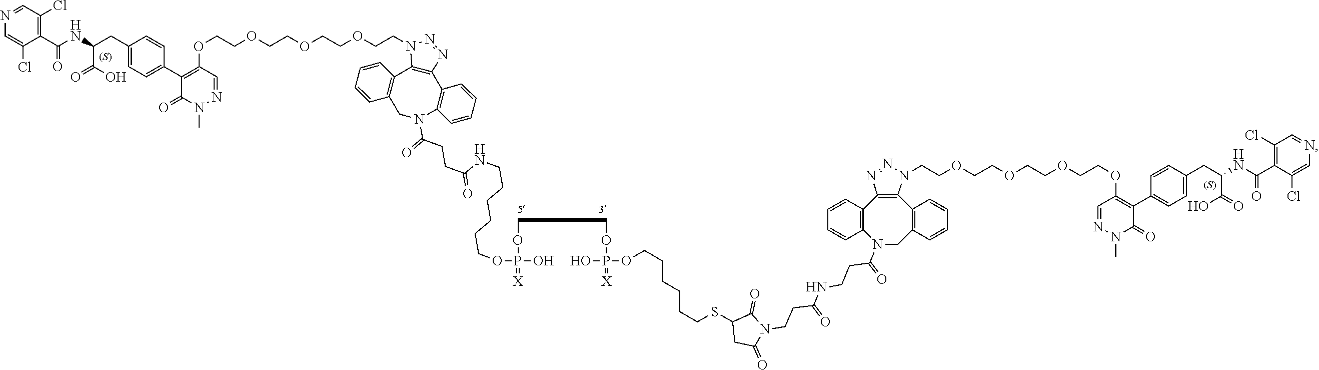

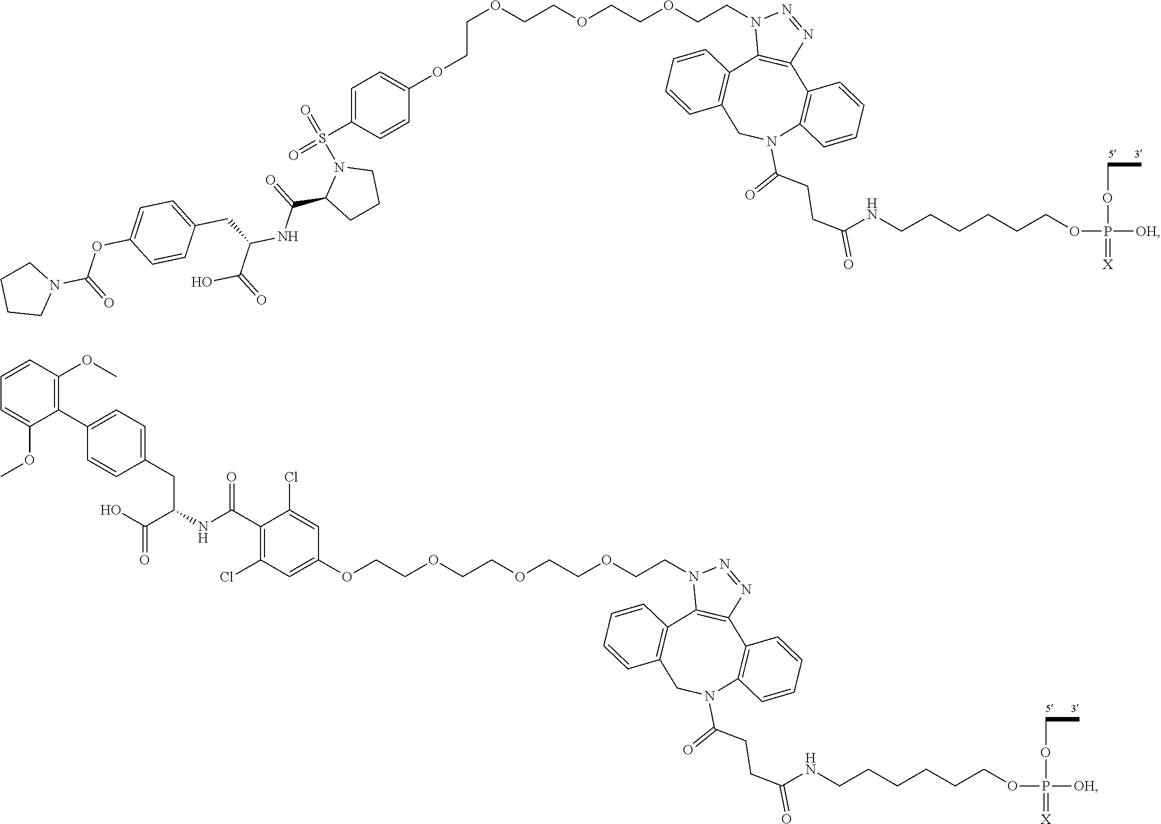

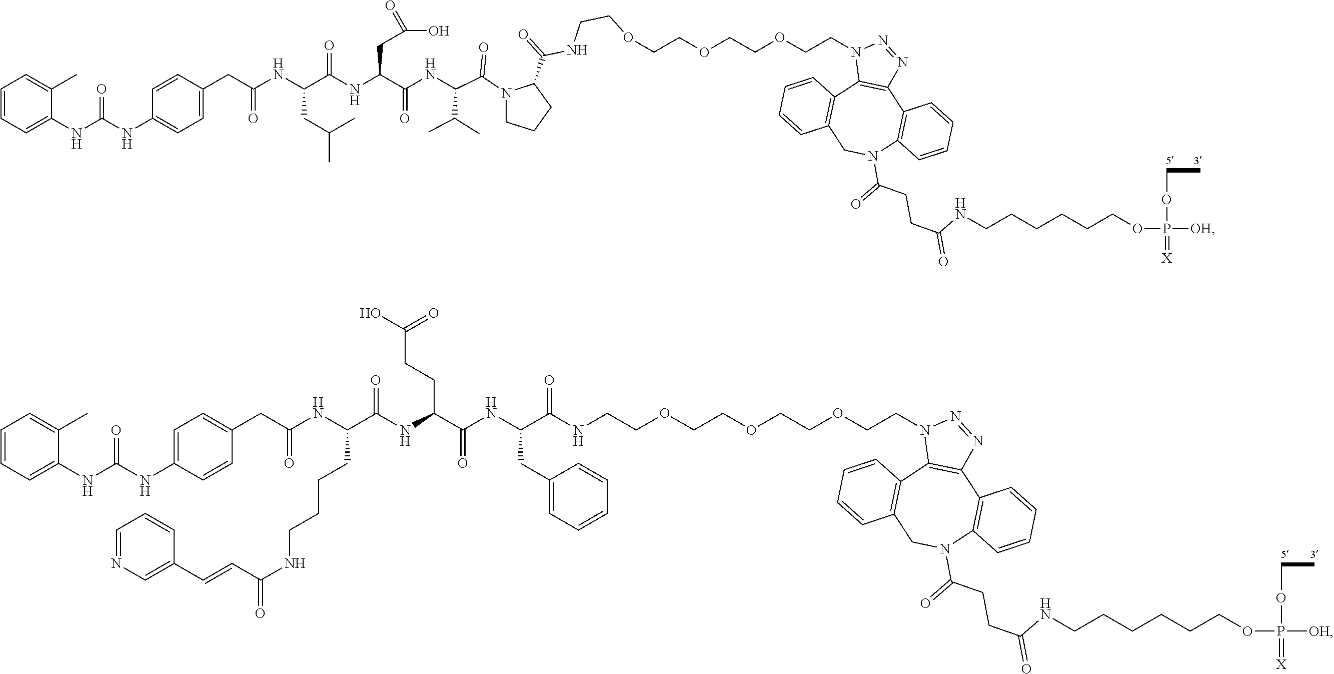

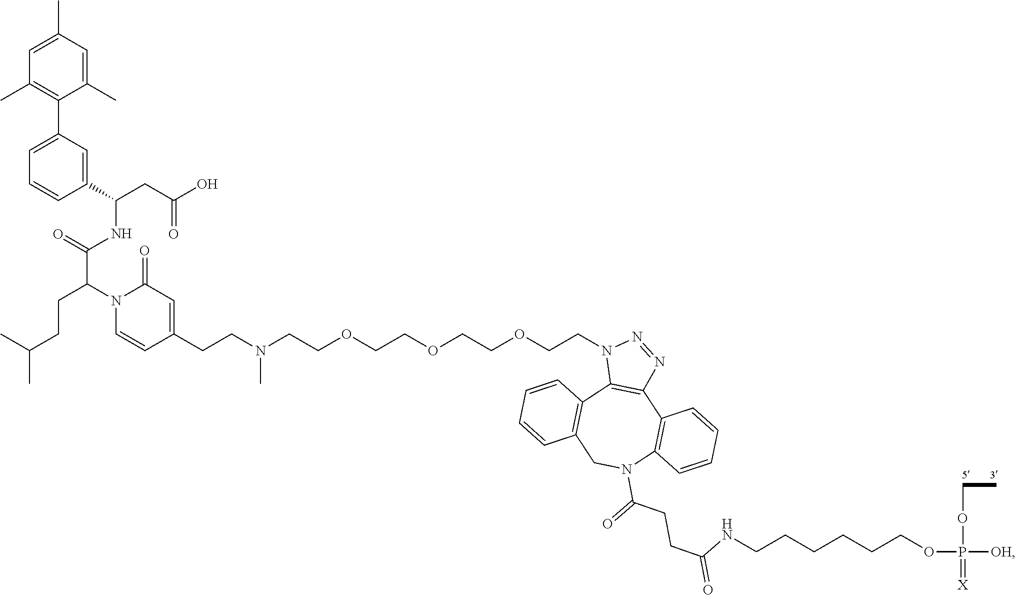

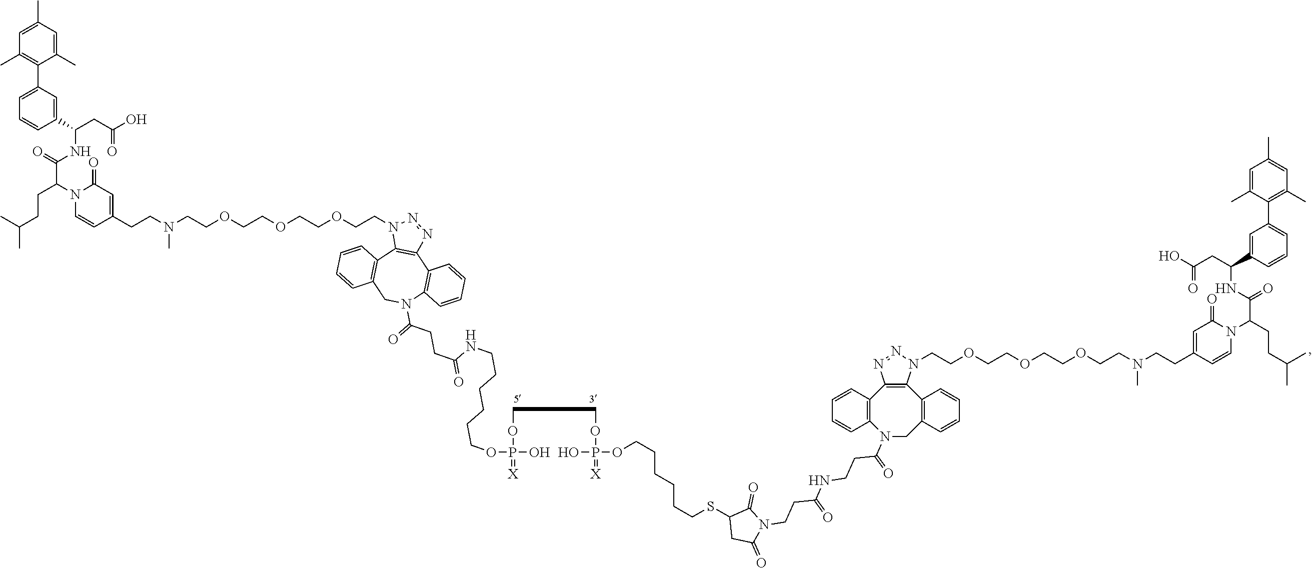

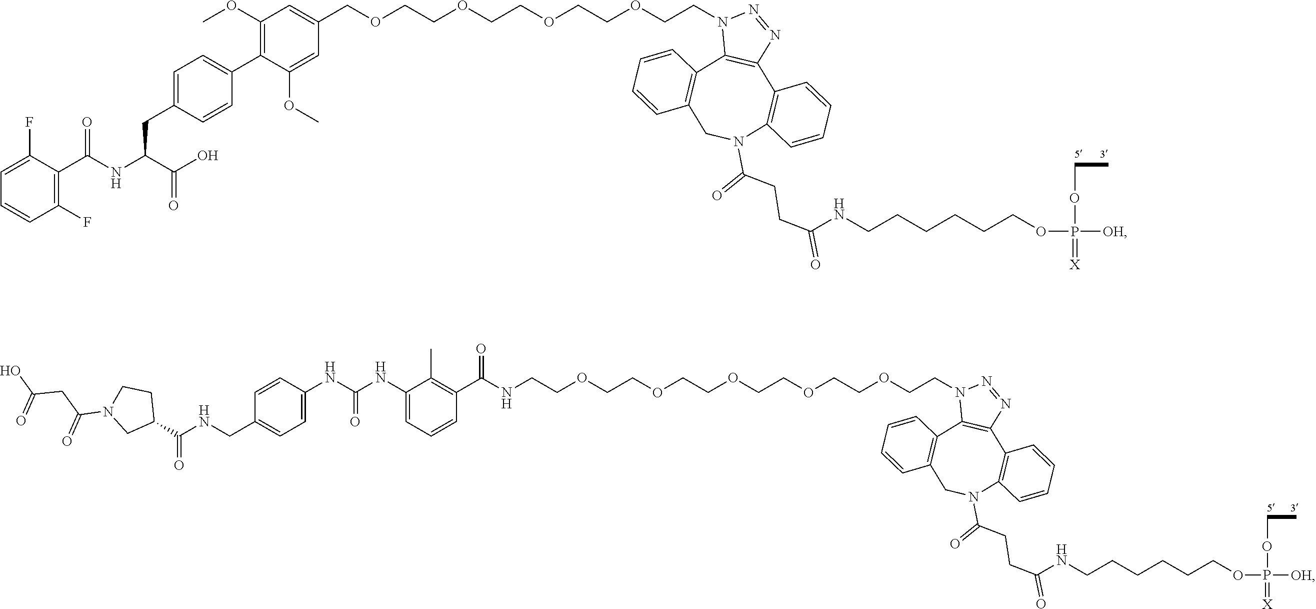

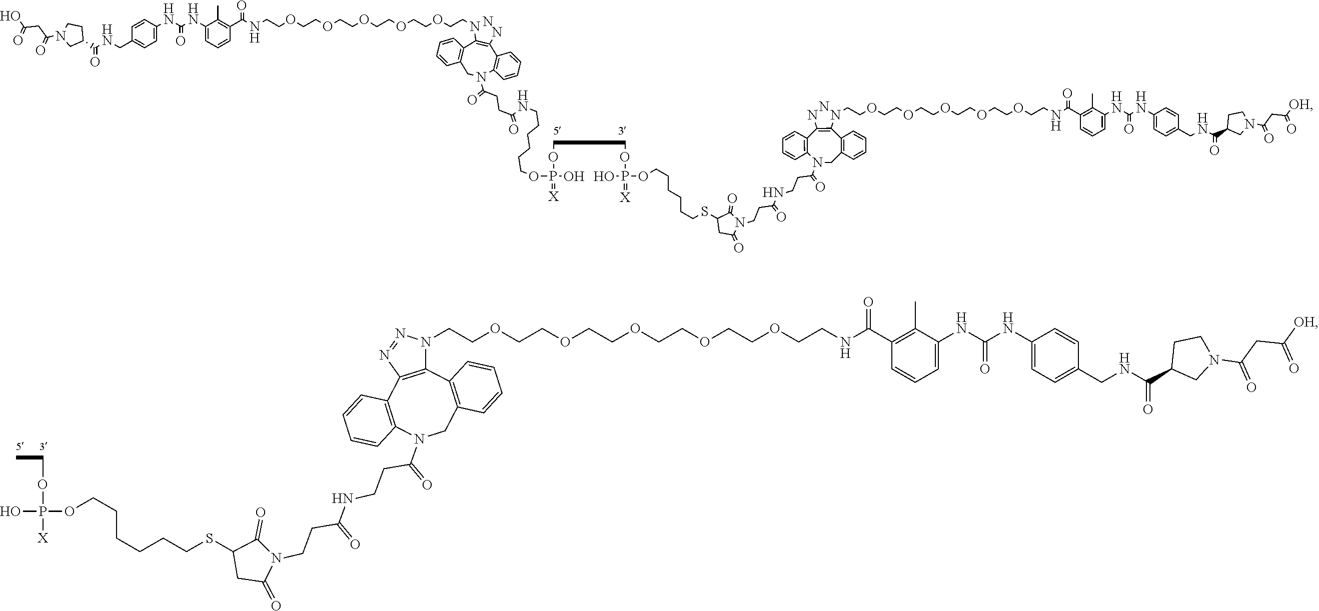

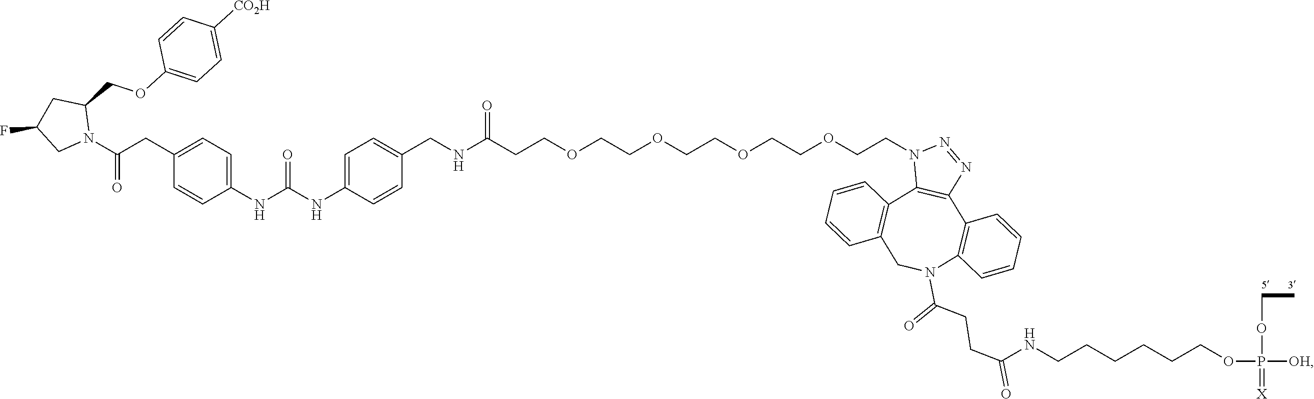

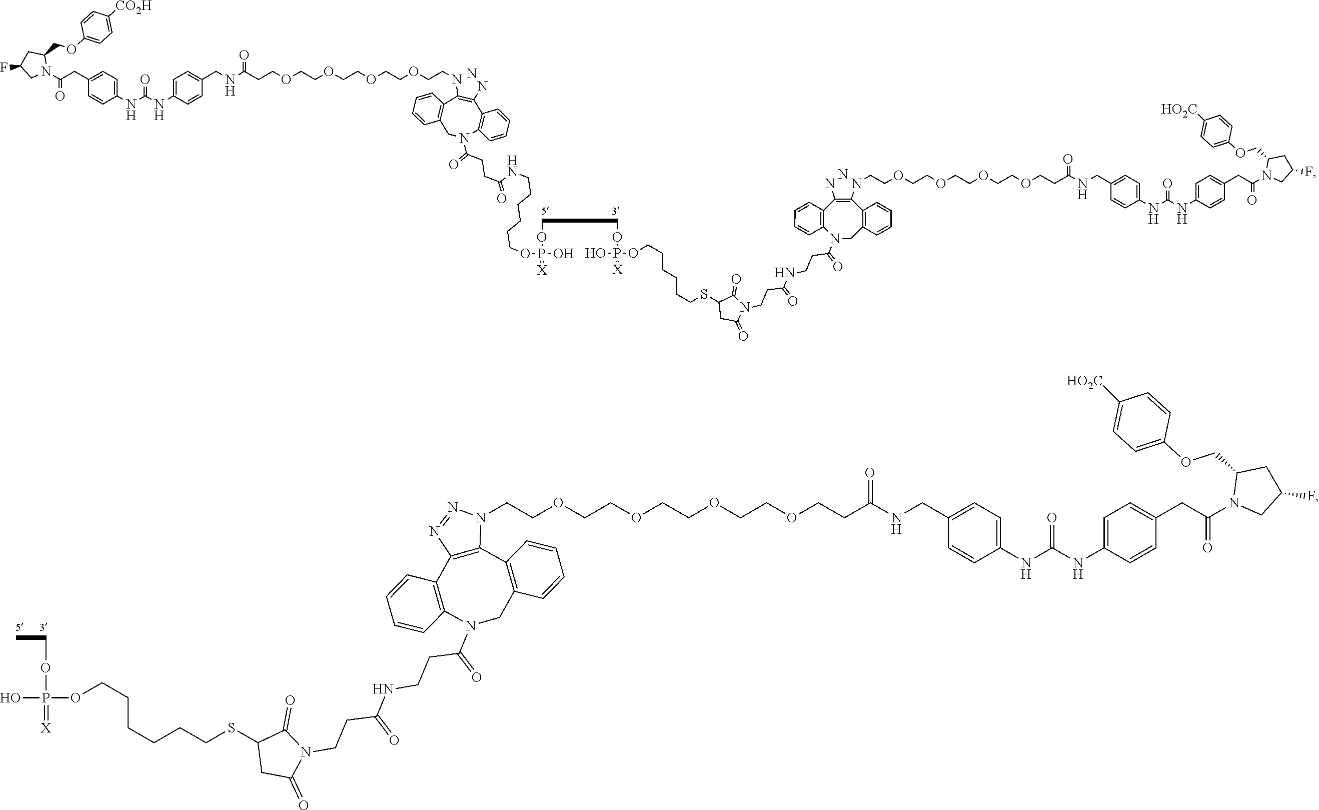

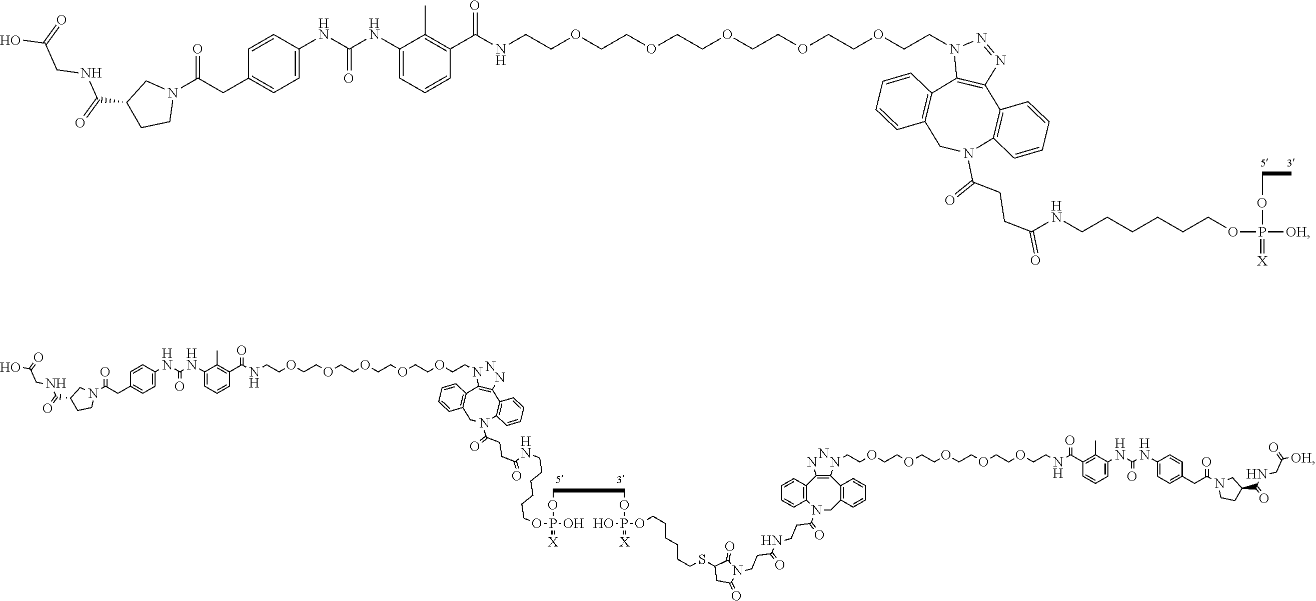

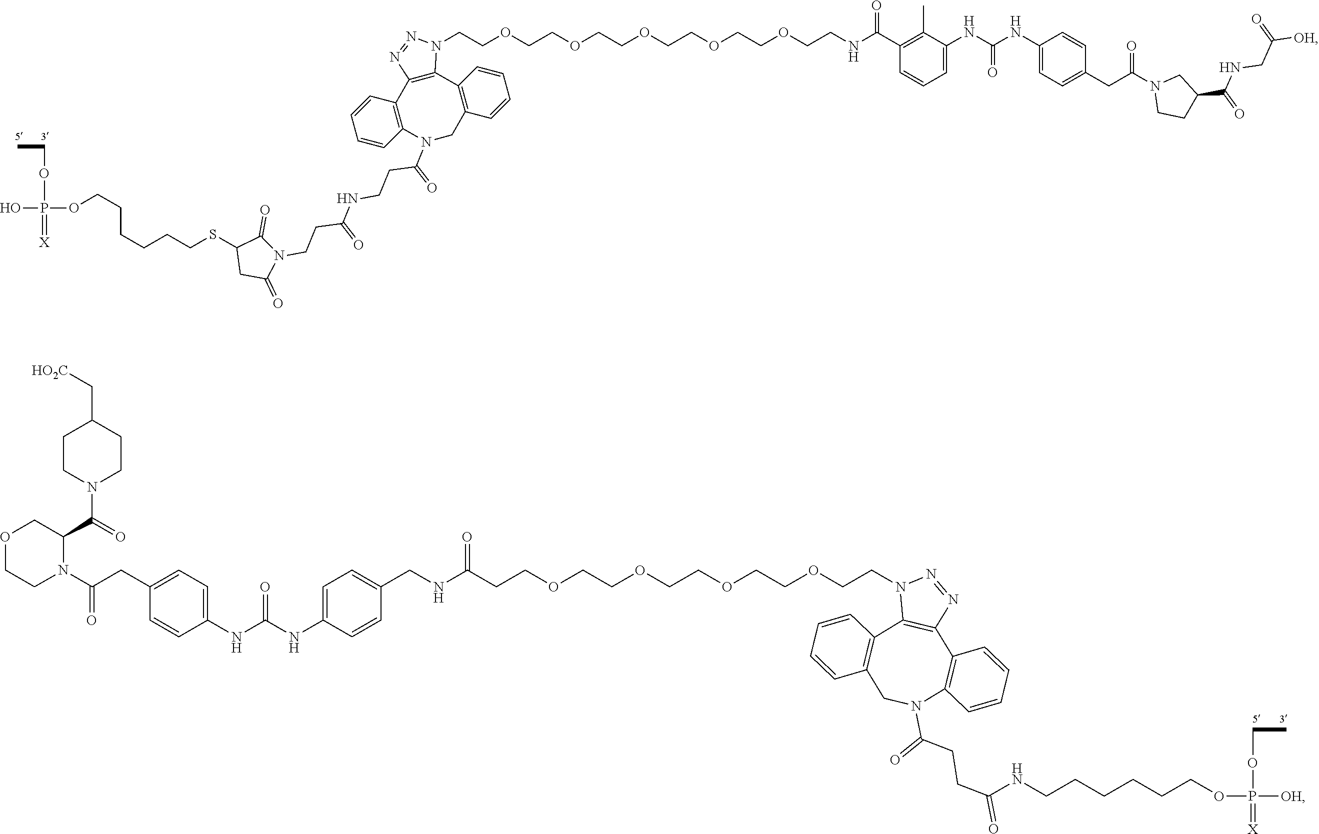

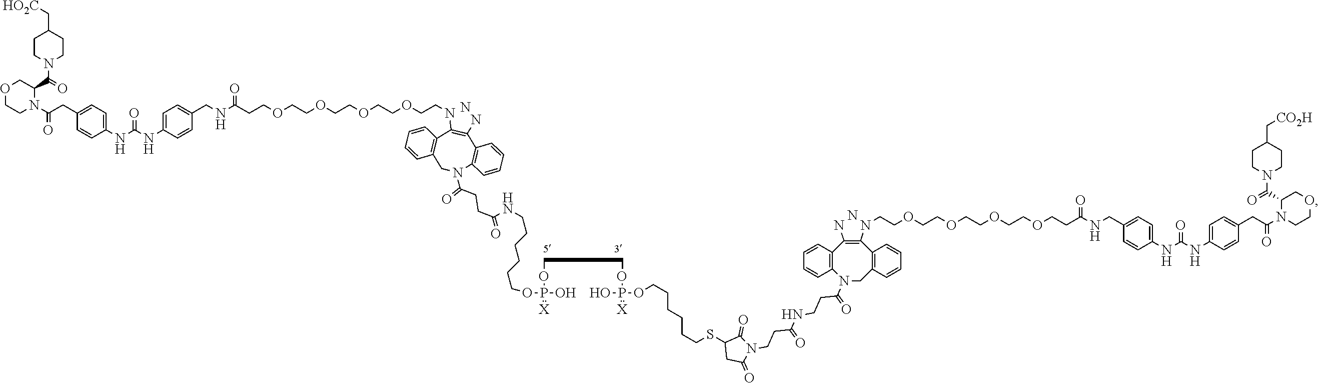

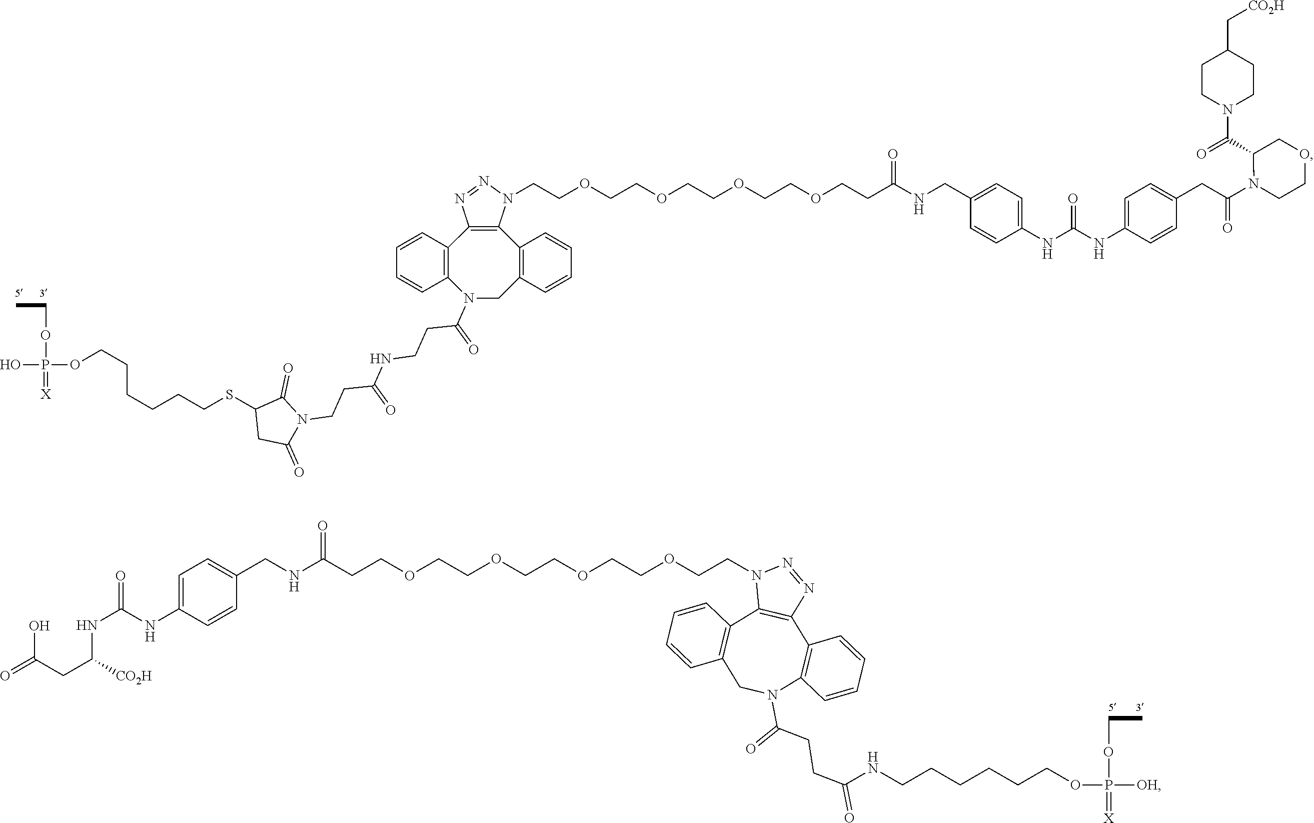

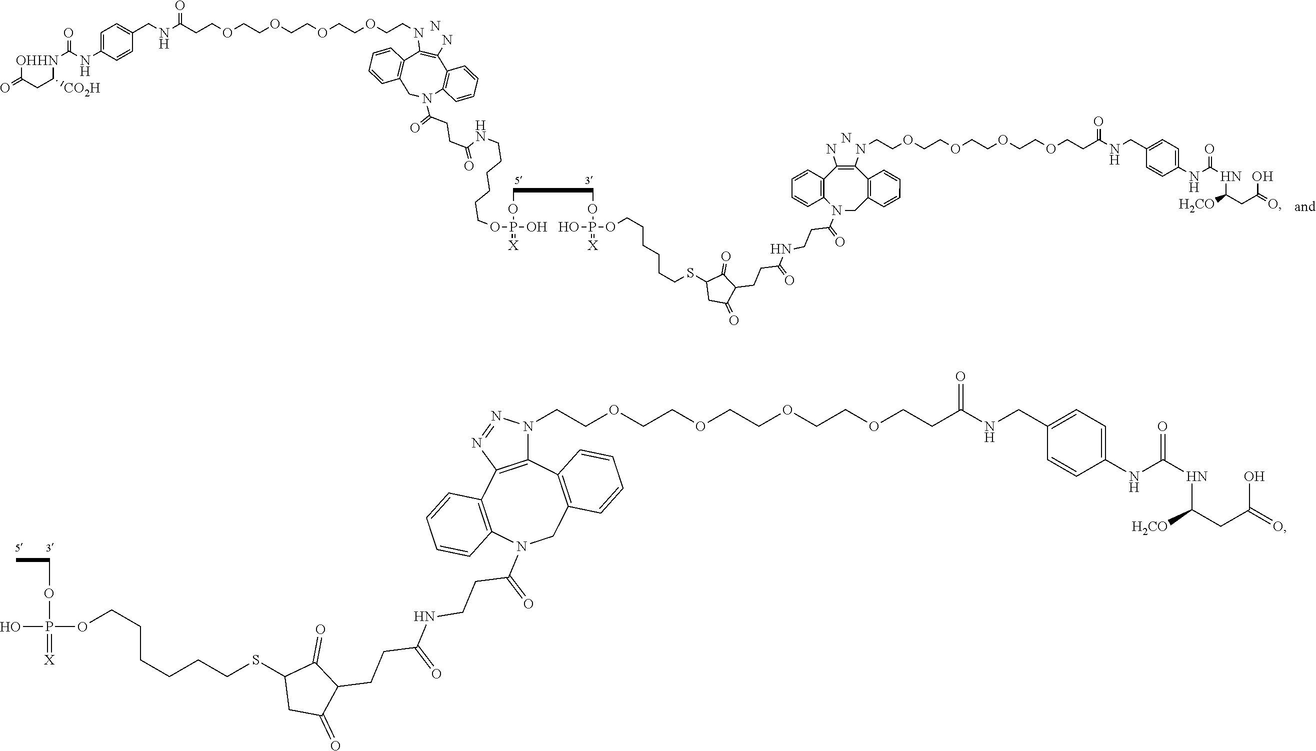

or a salt thereof, wherein X is O or S.

In some embodiments, X is O. In some embodiments, X is S.

In some embodiments, R¹ comprises an oligonucleotide. In some embodiments, the oligonucleotide is attached at its 5′ end. In some embodiments, the oligonucleotide is attached at its 3′ end. In some embodiments, the oligonucleotide is attached at an internal position on the oligonucleotide. In some embodiments the internal position is at an internucleoside linkage. In some embodiments, R¹ comprises an oligonucleotide conjugated to one or more additional α₄β_{1/7} integrin receptor ligands. In some embodiments, the oligonucleotide is conjugated to two, three, four, five, or more than five additional α₄β_{1/7} integrin receptor ligands. In certain embodiments, the additional α₄β_{1/7} integrin receptor ligands are conjugated to the oligonucleotide at the 5′ end of the oligonucleotide, the 3′ end of the oligonucleotide, one or more internal positions on the oligonucleotide, or any combination thereof. In certain embodiments, the oligonucleotide is a modified oligonucleotide.

Certain embodiments provide a composition comprising a compound of any embodiment herein, and a pharmaceutically acceptable carrier or excipient.

Certain embodiments provide a composition comprising a compound of any embodiment herein, for use in therapy.

In certain embodiments, a method for delivering an agent to cell comprises contacting the cell with the compound of any of the embodiments herein, thereby delivering the agent to the cell. In certain embodiments, the cell expresses α₄β₁₇ integrin receptor on the surface of the cell. In certain embodiments, the cell is a brain cell. In certain embodiments the cell is a cell of the frontal cortex. In certain embodiments, the cell is a cell of the striatum. In certain embodiments, the cell is a cell of the cerebellum. In certain embodiments, the cell is a cell of the brain stem. In certain embodiments, the cell is a cell of the hippocampus. In certain embodiments, the cell is a cell of the spinal cord. In certain embodiments, the agent is a therapeutic agent or diagnostic agent. In certain embodiments, the cell is in an animal.

In certain embodiments, a method of modulating the expression of a nucleic acid target in a cell comprises contacting the cell with the compound of any of the embodiments herein, thereby modulating expression of the nucleic acid target in the cell. In certain embodiments, the cell expresses α₄β_{1/7} integrin receptor on the surface of the cell. In certain embodiments, the cell is a brain cell. In certain embodiments the cell is a cell of the frontal cortex. In certain embodiments, the cell is a cell of the striatum. In certain embodiments, the cell is a cell of the cerebellum. In certain embodiments, the cell is a cell of the brain stem. In certain embodiments, the cell is a cell of the hippocampus. In certain embodiments, the cell is a cell of the spinal cord. In certain embodiments, the agent is a therapeutic agent or a diagnostic agent. In certain embodiments, contacting the cell with the compound of any of the embodiments herein inhibits expression of the nucleic acid target. In certain embodiments, the nucleic acid target is pre-mRNA, mRNA, non-coding RNA, or miRNA. In certain embodiments, the cell is in an animal.

In certain embodiments, a method of modulating the expression of a nucleic acid target in a subject comprises administering to the subject any of the compounds or compositions provided herein, thereby modulating expression of the nucleic acid target in the subject. In certain embodiments, the expression of the nucleic acid is modulated in a cell of the subject that expresses α₄β_{1/7} integrin receptor on the surface of the cell. In certain embodiments, the expression of the nucleic acid is modulated in a brain cell. In certain embodiments, the cell expressing α₄β_{1/7} integrin receptor on its surface is a brain cell. In certain embodiments, the brain cell is a cell of the frontal cortex. In certain embodiments, the brain cell is a cell of the striatum. In certain embodiments, the brain cell is a cell of the cerebellum. In certain embodiments, the brain cell is a cell of the brain stem. In certain embodiments, the brain cell is a cell of the hippocampus. In certain embodiments, the brain cell is a cell of the spinal cord. In certain embodiments, the nucleic acid target is pre-mRNA, mRNA, non-coding RNA, or miRNA. In certain embodiments, the compound is administered to the subject intrathecally.

In certain embodiments, a method of treating or ameliorating a disease, disorder, or symptom thereof in a subject, comprises administering to the subject any of the compounds or compositions provided herein, thereby treating, preventing, or ameliorating a disease, disorder, or symptom in the subject. In certain embodiments, the disease, disorder, or symptom thereof is a central nervous system (CNS) disease, disorder, or symptom thereof. In certain embodiments, the disease, disorder, or symptom thereof is Alzheimer's disease, or a symptom thereof. In certain embodiments, the compound is administered to the subject intrathecally. In certain embodiments, the compound or composition is administered to the subject in a therapeutically effective amount.

In certain embodiments, a compound comprising an α₄β_{1/7} integrin receptor ligand selectively or preferentially targets a cell expressing α₄β_{1/7} integrin receptor compared to a cell not expressing α₄β₁₇ integrin receptor. In certain embodiments, a compound comprising an α₄β₁₇ integrin receptor ligand selectively or preferentially targets a cell expressing α₄β_{1/7} integrin receptor compared to a compound not comprising an α₄β_{1/7} integrin receptor ligand.

Also provided herewith is the use of a compound as described herein for the manufacture of a medicament in the treatment of a disease or disorder.

In another aspect, the present disclosure provides methods for making any of the compounds provided herein, comprising one or more compounds and chemical transformations described herein, including Examples 1-13.

### Certain Compounds Comprising an Oligonucleotide

In certain embodiment, compounds described herein comprise oligonucleotides. In certain embodiments, an oligonucleotide has a nucleobase sequence that is at least partially complementary to a target nucleic acid sequence (e.g., an expressed target nucleic acid within a cell). In some embodiments, the oligonucleotide, upon delivery to a cell expressing a target nucleic acid, is able to modify the expression of the underlying gene. In some embodiments, the oligonucleotide, upon delivery to a cell expressing a target nucleic acid, is able to inhibit the expression of the underlying gene. The gene expression can be modified or inhibited in vitro or in vivo. In certain embodiments, an oligonucleotide comprises one or more ribonucleic acids (e.g., one or more ribonucleosides), deoxyribonucleic acids (e.g., one or more deoxyribonucleosides), modified nucleic acids (e.g., one or more modified nucleobases, sugars, and/or internucleoside linkages), or a combination thereof. In some embodiments, an oligonucleotide comprises a ribonucleic acid (RNA). In some embodiments, an oligonucleotide comprises a deoxyribonucleic acid (DNA). In some embodiments, an oligonucleotide comprises a modification (e.g., modified nucleobase, modified sugar, or modified internucleoside linkage).

In certain embodiments, an oligonucleotide is single-stranded. In some embodiments, a single-stranded oligonucleotide is single-stranded RNA (ssRNA), ssDNA, or a ssRNA/DNA hybrid (e.g., a single-stranded oligonucleotide comprised of both ribonucleosides (modified or unmodified) and deoxyribonucleosides (modified or unmodified))). In some embodiments, an oligonucleotide is double-stranded (e.g., comprised of two single-stranded nucleic acids). Such double-stranded oligonucleotides comprise a first oligonucleotide having a region complementary to a target nucleic acid and a second oligonucleotide having a region complementary to the first oligonucleotide. The first and second oligonucleotides can be independently modified. In certain embodiments, the first oligonucleotide is linked to one or more α₄β_{1/7} integrin receptor ligands. In certain embodiments, the second oligonucleotide is linked to one or more α₄β_{1/7} integrin receptor ligands.

In some embodiments, an oligonucleotide is at least 2 (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, or more) nucleotides in length. In some embodiments, an oligonucleotide is at least 5 nucleotides in length. In some embodiments, an oligonucleotide is at least 10 nucleotides in length. In some embodiments, an oligonucleotide is at least 15 nucleotides in length. In some embodiments, an oligonucleotide is at least 16 nucleotides in length. In some embodiments, an oligonucleotide is at least 17 nucleotides in length. In some embodiments, an oligonucleotide is at least 18 nucleotides in length. In some embodiments, an oligonucleotide is at least 19 nucleotides in length. In some embodiments, an oligonucleotide is at least 20 nucleotides in length. In some embodiments, an oligonucleotide is at least 21 nucleotides in length. In some embodiments, an oligonucleotide is at least 22 nucleotides in length. In some embodiments, an oligonucleotide is at least 23 nucleotides in length. In some embodiments, an oligonucleotide is at least 24 nucleotides in length. In some embodiments, an oligonucleotide is at least 25 nucleotides in length. In some embodiments, an oligonucleotide is at least 26 nucleotides in length. In some embodiments, an oligonucleotide is at least 27 nucleotides in length. In some embodiments, an oligonucleotide is at least 28 nucleotides in length. In some embodiments, an oligonucleotide is at least 29 nucleotides in length. In some embodiments, an oligonucleotide is at least 30 nucleotides in length. In some embodiments, an oligonucleotide is at least 40 nucleotides in length. In some embodiments, an oligonucleotide is at least 50 nucleotides in length. In some embodiments, an oligonucleotide is at least 60 nucleotides in length. In some embodiments, an oligonucleotide is at least 70 nucleotides in length. In some embodiments, an oligonucleotide is at least 80 nucleotides in length. In some embodiments, an oligonucleotide is at least 90 nucleotides in length. In some embodiments, an oligonucleotide is at least 100 nucleotides in length. In some embodiments, an oligonucleotide is at least 150 nucleotides in length.

In some embodiments, an oligonucleotide is less than or equal to 150 (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110,111,112,113,114,115,116,117,118,119,120,121,122,123,124,125,126,127,128, 129, 130, 131, 132, 133, 134, 135, 136, 137, 138, 139, 140, 141, 142, 143, 144, 145, 146, 147, 148, 149, 150) nucleotides in length. In some embodiments, an oligonucleotide is less than or equal to 150 nucleotides in length. In some embodiments, an oligonucleotide is less than or equal to 100 nucleotides in length. In some embodiments, an oligonucleotide is less than or equal to 90 nucleotides in length. In some embodiments, an oligonucleotide is less than or equal to 80 nucleotides in length. In some embodiments, an oligonucleotide is less than or equal to 70 nucleotides in length. In some embodiments, an oligonucleotide is less than or equal to 60 nucleotides in length. In some embodiments, an oligonucleotide is less than or equal to 50 nucleotides in length. In some embodiments, an oligonucleotide is less than or equal to 40 nucleotides in length. In some embodiments, an oligonucleotide is less than or equal to 30 nucleotides in length. In some embodiments, an oligonucleotide is less than or equal to 29 nucleotides in length. In some embodiments, an oligonucleotide is less than or equal to 28 nucleotides in length. In some embodiments, an oligonucleotide is less than or equal to 27 nucleotides in length. In some embodiments, an oligonucleotide is less than or equal to 26 nucleotides in length. In some embodiments, an oligonucleotide is less than or equal to 25 nucleotides in length. In some embodiments, an oligonucleotide is less than or equal to 24 nucleotides in length. In some embodiments, an oligonucleotide is less than or equal to 23 nucleotides in length. In some embodiments, an oligonucleotide is less than or equal to 22 nucleotides in length. In some embodiments, an oligonucleotide is less than or equal to 21 nucleotides in length. In some embodiments, an oligonucleotide is less than or equal to 20 nucleotides in length. In some embodiments, an oligonucleotide is less than or equal to 19 nucleotides in length. In some embodiments, an oligonucleotide is less than or equal to 18 nucleotides in length. In some embodiments, an oligonucleotide is less than or equal to 17 nucleotides in length. In some embodiments, an oligonucleotide is less than or equal to 16 nucleotides in length. In some embodiments, an oligonucleotide is less than or equal to 15 nucleotides in length. In some embodiments, an oligonucleotide is less than or equal to 10 nucleotides in length. In some embodiments, an oligonucleotide is less than or equal to 5 nucleotides in length.

In some embodiments, an oligonucleotide is about 5 nucleotides in length to about 150 nucleotides in length. In some embodiments, an oligonucleotide is about 10 nucleotides in length to about 100 nucleotides in length. In some embodiments, an oligonucleotide is about 20 nucleotides in length to about 90 nucleotides in length. In some embodiments, an oligonucleotide is about 30 nucleotides in length to about 80 nucleotides in length. In some embodiments, an oligonucleotide is about 40 nucleotides in length to about 70 nucleotides in length. In some embodiments, an oligonucleotide is about 50 nucleotides in length to about 60 nucleotides in length.

In some embodiments, an oligonucleotide is a therapeutic oligonucleotide. A therapeutic oligonucleotide may comprise, for example, without limitation, a small interfering RNA (siRNA), a microRNA (miRNA) antagonist, a miRNA mimic, an ADAR recruiting molecule, an ADAR targeting molecule, a guide RNA, an antisense oligonucleotide, a short hairpin RNA (shRNA), or combinations thereof.

In certain embodiments, a miRNA is a precursor, primary, and/or mature miRNA.

In certain embodiments, an oligonucleotide comprises or consists of an antisense oligonucleotide. In certain embodiments, an antisense oligonucleotide is complementary to an mRNA. In certain embodiments, an antisense oligonucleotide is complementary to a pre-mRNA. In certain embodiments, an antisense oligonucleotide blocks translation and promotes degradation of the mRNA transcript. In certain embodiments, an antisense oligonucleotide recruits RNase H and promotes degradation of the mRNA transcript. In certain embodiments, an antisense oligonucleotide targets miRNA, inhibiting the miRNA from modulating mRNA expression and promoting degradation of the miRNA.

### Certain Modifications

In certain aspects, the disclosure relates to compounds that comprise oligonucleotides. In certain embodiments, oligonucleotides may be unmodified RNA or DNA, or may be modified. In certain embodiments, the oligonucleotides are modified oligonucleotides. In certain embodiments, the modified oligonucleotides comprise at least one modified sugar, modified nucleobase, or modified internucleoside linkage relative to an unmodified RNA or DNA. In certain embodiments, an oligonucleotide has a modified nucleoside. A modified nucleoside may comprise a modified sugar, a modified nucleobase, or both a modified sugar and a modified nucleobase. Modified oligonucleotides may also include end modifications, e.g., 5′-end modifications and 3′-end modifications.

### Sugar Modifications and Motifs

In certain embodiments, a modified sugar is a substituted furanosyl sugar or non-bicyclic modified sugar. In certain embodiments, a modified sugar is a bicyclic or tricyclic modified sugar. In certain embodiments, a modified sugar is a sugar surrogate. A sugar surrogate may comprise one or more substitutions described herein.

In certain embodiments, a modified sugar is a substituted furanosyl or non-bicyclic modified sugar. In certain embodiments, the furanosyl sugar is a ribosyl sugar. In certain embodiments, the furanosyl sugar comprises one or more substituent groups, including, but not limited to, substituent groups at the 2′, 3′, 4′, and 5′ positions.

In certain embodiments, substituents at the 2′ position include, but are not limited to, F and OCH₃ (“OMe”, “O-methyl” or “methoxy”). In certain embodiments, substituent groups at the 2′ position suitable for non-bicyclic modified sugars include, but are not limited to, halo, allyl, amino, azido, SH, CN, OCN, CF₃, OCF₃, F, Cl, Br, SCH₃, SOCH₃, SO₂CH₃, ONO₂, NO₂, N₃, and NH₂. In certain embodiments, substituent groups at the 2′ position include, but are not limited to, O—(C₁-C₁₀) alkoxy, alkoxyalkyl, O-alkyl, S-alkyl, N-alkyl, O-alkenyl, S-alkenyl, N-alkenyl, O-alkynyl, S-alkynyl, N-alkynyl, O-alkyl-O-alkyl, alkynyl, wherein the alkyl, alkenyl and alkynyl can be substituted or unsubstituted C₁ to C₁₀ alkyl or C₂ to C₁₀ alkenyl and alkynyl. In certain embodiments, substituent groups at the 2′ position include, but are not limited to, alkaryl, aralkyl, O-alkaryl, and O-aralkyl. In certain embodiments, these 2′ substituent groups can be further substituted with one or more substituent groups independently selected from hydroxyl, alkoxy, carboxy, benzyl, phenyl, nitro (NO₂), thiol, thioalkoxy, thioalkyl, halogen, alkyl, aryl, alkenyl, and alkynyl. In certain embodiments, substituent groups at the 2′ position include, but are not limited to, O[(CH₂),O]ₘCH₃, O(CH₂),OCH₃, O(CH₂),CH₃, O(CH₂),ONH₂, O(CH₂),NH₂, O(CH₂).SCH₃, and O(CH₂),ON[(CH₂)·CH₃)]₂, where n and m are independently from 1 to about 10. In certain embodiments, substituent groups at the 2′ position include, but are not limited to, OCH₂CH₂OCH₃ (“MOE”), O(CH₂)₂ON(CH₃)₂(“DMAOE”), O(CH₂)₂O(CH₂)₂N(CH₃)₂ (“DMAEOE”), and OCH₂C(═O)—N(H)CH₃ (“NMA”).

In certain embodiments, substituent groups at the 4′ position suitable for non-bicyclic modified sugars include, but are not limited to, alkoxy (e.g., methoxy), alkyl, and those described in Manoharan et al., WO 2015/106128. In certain embodiments, substituent groups at the 5′ position suitable for non-bicyclic modified sugars include, but are not limited to, methyl (“Me”) (R or S), vinyl, and methoxy. In certain embodiments, one or more sugars comprise a 5′-vinylphosphonate modification. In certain embodiments, substituents described herein for the 2′, 4′, and 5′ position can be added to other specific positions on the sugar. In certain embodiments, such substituents may be added to the 3′ position of the sugar on the 3′ terminal nucleoside or the 5′ position of the 5′ terminal nucleoside. In certain embodiments, a non-bicyclic modified sugar may comprise more than one non-bridging sugar substituent. In certain such embodiments, non-bicyclic modified sugar substituents include, but are not limited to, 5′-Me-2′—F, 5′-Me-2′-OMe (including both R and S isomers). In certain embodiments, modified sugar substituents include those described in Migawa et al., WO 2008/101157 and Rajeev et al., US2013/0203836. In certain embodiments, substituent groups at the 5′ position suitable for non-bicyclic modified sugars include, but are not limited to, methyl (“Me” or “CH₃”) (R or S), vinyl, and methoxy. In certain embodiments, the 5′ modification is a 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-guanosine cap (7-methylated or non-methylated) (7m-G-O-5′-(HO)(O)P—O-(HO)(O)P—O—P(HO)(O)—O-5′); 5′adenosine cap (Appp), and any modified or unmodified nucleotide cap structure (N-O-5′(HO)(O)P—O—(HO)(O)P—O—P(HO)(O)—O-5′); 5′-monothiophosphate (phosphorothioate; (HO)₂(S)P—O-5′); 5′-monodithiophosphate (phosphorodithioate; (HO)(HS)(S)P—O-5′), 5′phosphorothiolate ((HO)₂(O)P-S-5′); any additional combination of oxygen/sulfur replaced monophosphate, diphosphate, and triphosphates (e.g., 5′-alpha-thiotriphosphate, 5′-gammathiotriphosphate, etc.), 5′-phosphoramidates ((HO)₂(O)P-NH-5′, (HO)(NH₂)(O)P—O-5′), 5′alkylphosphonates (R=alkyl=methyl, ethyl, isopropyl, propyl, etc., e.g., RP(OH)(O)—O-5′-, 5′alkenylphosphonates (i.e., vinyl, substituted vinyl), (OH)₂(O)P-5′-CH₂—), 5′alkyletherphosphonates (R=alkylether=methoxymethyl (MeOCH2-), ethoxymethyl, etc., e.g., RP(OH)(O)—O-5′-). In certain embodiments, one or more sugars comprise a 5′-vinylphosphonate modification. In certain embodiments the 5′ modification is at the terminus of an oligonucleotide. In certain embodiments the 5′ modification is at the terminus of an antisense oligonucleotide.

In certain embodiments, a modified sugar is a bicyclic sugar. A bicyclic sugar is a modified sugar comprising two rings, wherein the second ring is formed via a bridge connecting two of the atoms in the first ring, thereby forming a bicyclic structure. In certain embodiments, a bicyclic sugar comprises a bridging substituent that bridges two atoms of the furanosyl ring to form a second ring. In certain embodiments, a bicyclic sugar does not comprise a furanosyl moiety. A “bicyclic nucleoside” (“BNA”) is a nucleoside having a bicyclic sugar. In certain embodiments, the bicyclic sugar comprises a bridge between the 4′ and 2′ furanose ring atoms. In certain embodiments, the bicyclic sugar comprises a bridge between the 5′ and 3′ furanose ring atoms. In certain such embodiments, the furanose ring is a ribose ring. In certain embodiments, 4′ to 2′ bridging substituents include, but are not limited to, 4′-CH₂—2′, 4′—(CH₂)₂—2′, 4′—(CH₂)₃-2′, 4′-CH₂—O-2′ (“LNA”), 4′-CH₂—S-2′, 4′—(CH₂)₂—O-2′ (“ENA”), 4′-CH(CH₃)—O-2′ (“constrained ethyl” or “cEt” when in the S configuration), 4′-CH₂O—CH₂—2′, 4′-CH₂—N(R)-2′, 4′—CH(CH₂OCH₃)—O-2′ (“constrained MOE” or “cMOE”) and analogs thereof (e.g., U.S. Pat. No. 7,399,845), 4′-C(CH₃)(CH₃)—O-2′ and analogs thereof (e.g., U.S. Pat. No. 8,278,283), 4′-CH₂—N(OCH₃)-2′ and analogs thereof (e.g., U.S. Pat. No. 8,278,425), 4′-CH₂—O—N(CH₃)-2′ (e.g., U.S. Patent Publication No. 2004/0171570), 4′-CH₂—N(R)—O-2′, wherein R is H, C₁-C₁2 alkyl, or a protecting group (e.g., U.S. Pat. No. 7,427,672), 4′-CH₂—C(H)(CH₃)-2′ (e.g., Chattopadhyaya et al., J. Org. Chem., 2009, 74, 118-134), and 4′-CH₂—C(═CH₂)-2′ and analogs thereof (e.g., U.S. Pat. No. 8,278,426). The entire contents of each of the foregoing are hereby incorporated herein by reference. Additional representative U.S. Patents and U.S. Patent Publications that teach the preparation of bicyclic nucleic acid nucleotides include, but are not limited to, the following: U.S. Pat. Nos. 6,268,490; 6,525,191; 6,670,461; 6,770,748; 6,794,499; 6,998,484; 7,053,207; 7,034,133;7,084,125; 7,399,845; 7,427,672; 7,569,686; 7,741,457; 8,022,193; 8,030,467; 8,278,425; 8,278,426; 8,278,283; US 2008/0039618; and US 2009/0012281, US 2013/0190383; and WO 2013/036868, the entire contents of each of which are hereby incorporated herein by reference. Any of the foregoing bicyclic nucleosides can be prepared having one or more stereochemical sugar configurations including, for example, a-L-ribofuranose and 3-D-ribofuranose (see, e.g., WO 99/14226). Specified bicyclic nucleosides herein are in the 3-D configuration, unless otherwise specified.

In certain embodiments, a modified sugar is a sugar surrogate. In certain embodiments, a sugar surrogate has the oxygen atom replaced, e.g., with a sulfur, carbon or nitrogen atom. In certain such embodiments, the sugar surrogate may also comprise bridging and/or non-bridging substituents as described herein. In certain embodiments, sugar surrogates comprise rings having other than 5 atoms. In certain such embodiments, the sugar surrogate comprises a cyclobutyl moiety in place of the pentofuranosyl sugar. In certain embodiments, the sugar surrogate comprises a six membered ring in place of the pentofuranosyl sugar. In certain embodiments, the sugar surrogate comprises a tetrahydropyran (“THP”) in place of the pentofuranosyl sugar. In certain embodiments, the sugar surrogate comprises a morpholino in place of the pentofuranosyl sugar. Representative U.S. patents that teach the preparation of such modified sugar structures include, but are not limited to, U.S. Pat. Nos. 4,981,957; 5,118,800; 5,166,315; 5,185,444; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,700,920; 7,875,733; 7,939,677, 8,088,904; 8,440,803; and 9,005,906, the entire contents of each of the foregoing are hereby incorporated herein by reference.

In some embodiments, sugar surrogates comprise acyclic moieties. In certain embodiments, the sugar surrogate is an unlocked nucleic acid (“UNA”). A UNA is unlocked acyclic nucleic acid, wherein any of the bonds of the sugar has been removed, forming an unlocked “sugar” residue. In one example, UNA also encompasses a monomer where the bonds between C₁′-C₄′ have been removed (i.e., the covalent carbon-oxygen-carbon bond between the C₁′ and C₄′ carbons). In another example, the C2′-C3′ bond (i.e., the covalent carbon-carbon bond between the C2′ and C3′ carbons) of the sugar has been removed. Representative U.S. publications that teach the preparation of UNA include, but are not limited to, U.S. Pat. No. 8,314,227; and U.S. Patent Publication Nos. 2013/0096289; 2013/0011922; and 2011/0313020, the entire contents of each of which are hereby incorporated herein by reference. In certain embodiments, sugar surrogates comprise peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., US2013/130378, the entire contents of which is incorporated herein by reference. Many other bicyclic and tricyclic sugar and sugar surrogate ring systems are known in the art that can be used in modified nucleosides.

In certain aspects, the disclosure relates to compounds comprising at least one oligonucleotide, wherein the nucleosides of such oligonucleotides comprise one or more types of modified sugars and/or unmodified sugars arranged along the oligonucleotide or region thereof in a defined pattern or “sugar motif”. In certain instances, such sugar motifs include, but are not limited to, any of the patterns of sugar modifications described herein.

In certain embodiments, an oligonucleotide comprises a gapmer sugar motif. A gapmer oligonucleotide comprises or consists of a region having two external “wing” regions and a central or internal “gap” region. The gap and wing regions form a contiguous sequence of nucleosides, wherein the majority of nucleoside sugars of each of the wings differ from the majority of nucleoside sugars of the gap. In certain embodiments, the wing regions comprise a majority of modified sugars, and the gap comprises a majority of unmodified sugars. In certain embodiments, the nucleosides of the gap are deoxynucleosides. Compounds with a gapmer sugar motif are described in, for example, U.S. Pat. No. 8,790,919, the contents of which is incorporated herein by reference.

In certain embodiments, one or both oligonucleotides of a double-stranded compound comprise a triplet sugar motif. An oligonucleotide with a triplet sugar motif comprises three identical sugar modifications on three consecutive nucleosides. In certain embodiments, the triplet is at or near the cleavage site of the oligonucleotide. In certain embodiments, an oligonucleotide of a double-stranded compound may contain more than one triplet sugar motif. In certain embodiments, the identical sugar modification of the triplet sugar motif is a 2′-F modification. Compounds with a triplet sugar motif are disclosed, for example, in U.S. Pat. No. 10,668,170, the contents of which is incorporated herein by reference.

In certain embodiments, one or both oligonucleotides of a double-stranded compound comprise a quadruplet sugar motif. An oligonucleotide with a quadruplet sugar motif comprises four identical sugar modifications on four consecutive nucleosides. In certain embodiments, the quadruplet is at or near the cleavage site. In certain embodiments, an oligonucleotide of a double-stranded compound may contain more than one quadruplet sugar motif. In certain embodiments, the identical sugar modification of the quadruplet sugar motif is a 2′-F modification. For a double-stranded compound having a duplex region of 19-23 nucleotides in length, the cleavage site of the antisense oligonucleotide is typically around the 10, 11, and 12 positions from the 5′-end. In certain embodiments, the quadruplet sugar motif is at the 8, 9, 10, 11 positions; the 9, 10, 11, 12 positions; the 10, 11, 12, 13 positions; the 11, 12, 13, 14 positions; or the 12, 13, 14, 15 positions of the sense oligonucleotide, counting from the first nucleoside of the 5′-end of the sense oligonucleotide, or, the count starting from the first paired nucleotide within the duplex region from the 5′-end of the sense oligonucleotide. In certain embodiments, the quadruplet sugar motif is at the 8, 9, 10, 11 positions; the 9, 10, 11, 12 positions; the 10, 11, 12, 13 positions; the 11, 12, 13, 14 positions; or the 12, 13, 14, 15 positions of the antisense oligonucleotide, counting from the first nucleoside of the 5′-end of the antisense oligonucleotide, or, the count starting from the first paired nucleotide within the duplex region from the 5′-end of the antisense oligonucleotide. The cleavage site may change according to the length of the duplex region of the double-stranded compound and may change the position of the quadruplet accordingly.

In certain embodiments, an oligonucleotide comprises an alternating sugar motif. In certain embodiments, one or both oligonucleotides of a double-stranded compound comprise an alternating sugar motif. An oligonucleotide with an alternating sugar motif comprises at least two different sugar modifications, wherein one or more consecutive nucleosides comprising a first sugar modification alternates with one or more consecutive nucleosides comprising a second sugar modification, and one or more consecutive nucleosides comprising a third sugar modification, etc. For example, if A, B, and C each represent one type of modification to the nucleoside, the alternating motif can be “ABABABABABAB . . . ,” “AABBAABBAABB . . . ,” “AABAABAABAAB “AAABAAABAAAB . . . ,” “AAABBBAAABBB . . . ,” or “ABCABCABCABC . . . ” etc. In certain embodiments, the alternating sugar motif is repeated for at least 2, 3, 4, 5, 6, 7, 8, 9, 10, 11, 12, 13, 14, 15, 16, 17, 18, 19, 20, 21, 22, or 23 contiguous nucleobases along an oligonucleotide. In certain embodiments, the alternating sugar motif is comprised of two different sugar modifications. In certain embodiments, the alternating sugar motif comprises 2′—OMe and 2′-F sugar modifications.

In certain embodiments, each nucleoside of an oligonucleotide is independently modified with one or more sugar modifications provided herein. In certain embodiments, each oligonucleotide of a double-stranded compound independently has one or more sugar motifs provided herein. In certain embodiments, an oligonucleotide containing a sugar motif is fully modified in that each nucleoside other than the nucleosides comprising the sugar motif comprises a sugar modification.

### Nucleobase Modifications and Motifs

In certain embodiments, modified oligonucleotides comprise one or more nucleosides comprising a modified nucleobase. In certain embodiments, modified oligonucleotides comprise one or more nucleosides that do not comprise a nucleobase, referred to as an abasic nucleoside.

In certain embodiments, modified nucleobases are selected from: 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 2-aminopropyladenine, 5-hydroxymethyl cytosine, 5-methylcytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (C—C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly, 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size expanded bases, and fluorinated bases. Further modified nucleobases include tricyclic pyrimidines, such as 1,3-diazaphenoxazine-2-one, 1,3-diazaphenothiazine-2-one, and 9-(2-aminoethoxy)-1,3-diazaphenoxazine-2-one (G-clamp). Modified nucleobases may also include those in which the purine or pyrimidine base is replaced with other heterocycles, for example, 7-deaza-adenine, 7-deazaguanosine, 2-aminopyridine and 2-pyridone.

Further nucleobases include those disclosed in U.S. Pat. No. 3,687,808; Modified Nucleosides in Biochemistry, Biotechnology and Medicine, Herdewijn, P. ed. Wiley-VCH, 2008; The Concise Encyclopedia Of Polymer Science And Engineering, pages 858-859; Kroschwitz, J. L., Ed., John Wiley & Sons, 1990, 858-859; Englisch et al., Angewandte Chemie, International Edition, 1991, 30, 613; Sanghvi, Y. S., Chapter 15, dsRNA Research and Applications, pages 289-302; Antisense Research and Applications, Crooke, S. T. and Lebleu, B., Eds., CRC Press, 1993, 273-288; Antisense Drug Technology, Crooke S.T., Ed., CRC Press, 2008, 163-166 and 442-443 (Chapters 6 and 15), each of which are incorporated herein by reference.

Publications that teach the preparation of certain of the above noted modified nucleobases, as well as other modified nucleobases, include without limitation, U.S. Patent Application Publication Nos. 2003/0158403 and 2003/0175906; U.S. Pat. Nos. 4,845,205; 5,130,302; 5,134,066; 5,175,273; 5,367,066; 5,432,272; 5,434,257; 5,457,187; 5,459,255; 5,484,908; 5,502,177; 5,525,711; 5,552,540; 5,587,469; 5,594,121; 5,596,091; 5,614,617; 5,645,985; 5,681,941; 5,811,534; 5,750,692; 5,948,903; 5,587,470; 5,457,191; 5,763,588; 5,830,653; 5,808,027; 6,005,096. 6,015,886; 6,147,200; 6,166,197; 6,166,199; 6,222,025; 6,235,887; 6,380,368; 6,528,640; 6,639,062; 6,617,438; 7,045,610; 7,427,672; and 7,495,088, the contents of each of which are incorporated herein by reference.

In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines.

In certain embodiments, modified oligonucleotides comprise a block of modified nucleobases. In certain such embodiments, the block is at the 3′-end of the oligonucleotide. In certain embodiments, the block is within 3 nucleosides of the 3′-end of the oligonucleotide. In certain embodiments, the block is at the 5′-end of the oligonucleotide. In certain embodiments, the block is within 3 nucleosides of the 5′-end of the oligonucleotide.

### Internucleoside Linkage Modifications and Motifs

A 3′ to 5′ phosphodiester linkage is the naturally occurring internucleoside linkage of RNA and DNA. In certain embodiments, an oligonucleotide has one or more modified, i.e., non-naturally occurring, internucleoside linkages. Certain non-naturally occurring internucleoside linkages may impart desirable properties such as, for example, enhanced cellular uptake, enhanced affinity for target nucleic acids, and increased stability in the presence of nucleases. Representative phosphorus-containing modified internucleoside linkages include, but are not limited to, phosphotriesters, alkylphosphonates (e.g., methylphosphonates), phosphoramidates, and phosphorothioates (“P=S”), and phosphorodithioates (“HS-P=S”). Representative non-phosphorus containing internucleoside linking groups include, but are not limited to, methylenemethylimino (—CH₂—N(CH₃)—O—CH₂), thiodiester, thionocarbamate (—O—C(═O)(NH)—S—); siloxane (—O—SiH₂—O-); and N,N′-dimethylhydrazine (—CH₂—N((CH₃)—N((CH₃)—). Methods of preparation of phosphorous-containing and non-phosphorous-containing internucleoside linkages are well known to those skilled in the art. Neutral internucleoside linkages include, without limitation, phosphotriesters, methylphosphonates, MMI (3′—CH₂—N(CH₃)—O-5′), amide-3 (3′—CH₂— C(=O)—N(H)-5′), amide-4 (3′—CH₂—N(H)-C(═O)-5′), formacetal (3′—O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See, for example: Carbohydrate Modifications in Antisense Research; Y. S. Sanghvi and P.D. Cook, Eds., ACS Symposium Series 580; Chapters 3 and 4, 40-65). Further neutral internucleoside linkages include nonionic linkages comprising mixed N, O, S and CH₂ component parts.

In certain embodiments, an oligonucleotide comprises at least one modified internucleoside linkage. A modified internucleoside linkage may be placed at any position of an oligonucleotide. For double-stranded compounds, a modified internucleoside linkage may be placed within the sense oligonucleotide, antisense oligonucleotide, or both oligonucleotides of the double-stranded compound.

In certain embodiments, the internucleoside linkage modification may occur on every nucleoside of an oligonucleotide. In certain embodiments, internucleoside linkage modifications may occur in an alternating pattern along an oligonucleotide. In certain embodiments, essentially each internucleoside linking group is a phosphate internucleoside linkage (P=0). In certain embodiments, each internucleoside linking group of a modified oligonucleotide is a phosphorothioate (P=S). In certain embodiments, each internucleoside linking group of a modified oligonucleotide is independently selected from a phosphorothioate and phosphate internucleoside linkage. In certain embodiments, the pattern of the internucleoside linkage modification on each oligonucleotide of a double-stranded compound is the same. In certain embodiments, the pattern of the internucleoside linkage modification on each oligonucleotide of a double-stranded compound is different. In certain embodiments, a double-stranded compound comprises 6-8 modified internucleoside linkages. In certain embodiments, the 6-8 modified internucleoside linkages are phosphorothioate internucleoside linkages or alkylphosphonate internucleoside linkages. In certain embodiments, the sense oligonucleotide comprises at least two modified internucleoside linkages at either or both the 5′-end and the 3′-end. In certain such embodiments, the modified internucleoside linkages are phosphorothioate internucleoside linkages or alkylphosphonate internucleoside linkages. In certain embodiments, the antisense oligonucleotide comprises at least two modified internucleoside linkages at either or both the 5′-end and the 3′-end. In certain such embodiments, the modified internucleoside linkages are phosphorothioate internucleoside linkages or alkylphosphonate internucleoside linkages.

In certain embodiments, a double-stranded compound comprises an overhang region. In certain embodiments, a double-stranded compound comprises a phosphorothioate or alkylphosphonate internucleoside linkage modification in the overhang region. In certain embodiments, a double-stranded compound comprises a phosphorothioate or alkylphosphonate internucleotide linkage linking the overhang nucleotide with a paired nucleotide that is next to the overhang nucleotide. For instance, there may be at least two phosphorothioate internucleoside linkages between the terminal three nucleosides, in which two of the three nucleosides are overhang nucleosides, and the third is a paired nucleoside next to the overhang nucleoside. These terminal three nucleosides may be at the 3′-end of the antisense oligonucleotide, the 3′-end of the sense oligonucleotide, the 5′-end of the antisense oligonucleotide, or the 5′-end of the antisense oligonucleotide.

In certain embodiments, modified oligonucleotides comprise one or more internucleoside linkages having chiral centers. Representative chiral internucleoside linkages include, but are not limited to, alkylphosphonates and phosphorothioates. Modified oligonucleotides comprising internucleoside linkages having chiral centers can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. As is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration.

α₄β_{1/7} Integrin Receptor Ligands

In some embodiments, the compounds provided herein comprise an α₄β₁₇ integrin receptor ligand. In some embodiments, an α₄β_{1/7} integrin receptor ligand is useful for directing a therapeutic, prophylactic, or diagnostic agent. In certain embodiments, a therapeutic agent is an oligonucleotide (e.g., a therapeutic oligonucleotide). In some embodiments, an α₄β_{1/7} integrin receptor ligand directs an oligonucleotide to a locality. In some embodiments, an α₄β_{1/7} integrin receptor ligand targets tissues. In some embodiments, the tissue is brain tissue. In some embodiments, an α₄β_{1/7} integrin receptor ligand targets a cell receptor. In some embodiments, a cell receptor is an α₄β₁₇ integrin receptor. In some embodiments, an α₄β_{1/7} integrin receptor is in the brain. In some embodiments, an α₄β_{1/7} integrin receptor is in the frontal cortex. In some embodiments, an α₄β_{1/7} integrin receptor is in the striatum. In some embodiments, an α₄β_{1/7} integrin receptor is in the cerebellum. In some embodiments, an α₄β_{1/7} integrin receptor is in the brain stem. In some embodiments, an α₄β_{1/7} integrin receptor is in the hippocampus. In some embodiments, an α₄β_{1/7} integrin receptor is in the spinal cord.

The use of any α₄β_{1/7} integrin receptor ligand in the compounds provided herein is contemplated by the present disclosure. α₄β_{1/7} integrin receptor ligands are known in the art, and a person of ordinary skill in the art would be capable of identifying additional α₄β_{1/7} integrin receptor ligands for use in the compounds described herein beyond those explicitly provided by the present disclosure. The present disclosure also contemplates the use of derivatives and prodrugs of any α₄β_{1/7} integrin receptor ligand provided herein or known in the art in the presently described compounds, and a person of ordinary skill in the art would know how to make such derivatives and prodrugs.

In some embodiments, an α₄β_{1/7} integrin receptor ligand is an α₄β_{1/7} integrin receptor agonist. In some embodiments, an α₄β_{1/7} integrin receptor ligand is an α₄β₁₇ integrin receptor antagonist. In some embodiments, an α₄β_{1/7} integrin receptor ligand is any of those disclosed in International Patent Application Publication No. WO 2019/246455, which is incorporated herein by reference. In some embodiments, an α₄β_{1/7} integrin receptor ligand is any of those disclosed in Baiula, M. et al. Novel Ligands Targeting α₄β_{1/7} Integrin: Therapeutic Applications and Perspectives. Front. Chem. 2019, 7, 489, which is incorporated herein by reference. Exemplary a(4Pin1 integrin receptor ligands for use in the present disclosure include, but are not limited to, any of the following α₄β_{1/7} in integrin receptor ligands, and derivatives thereof:

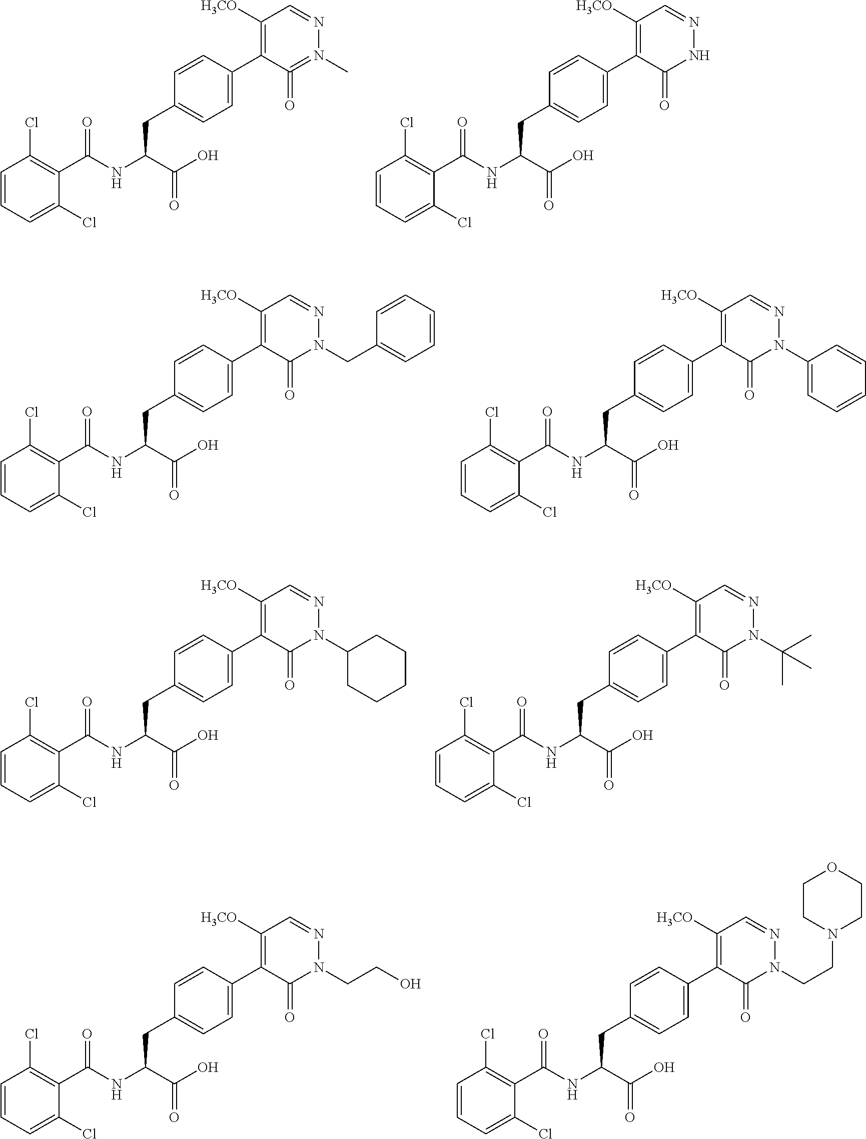

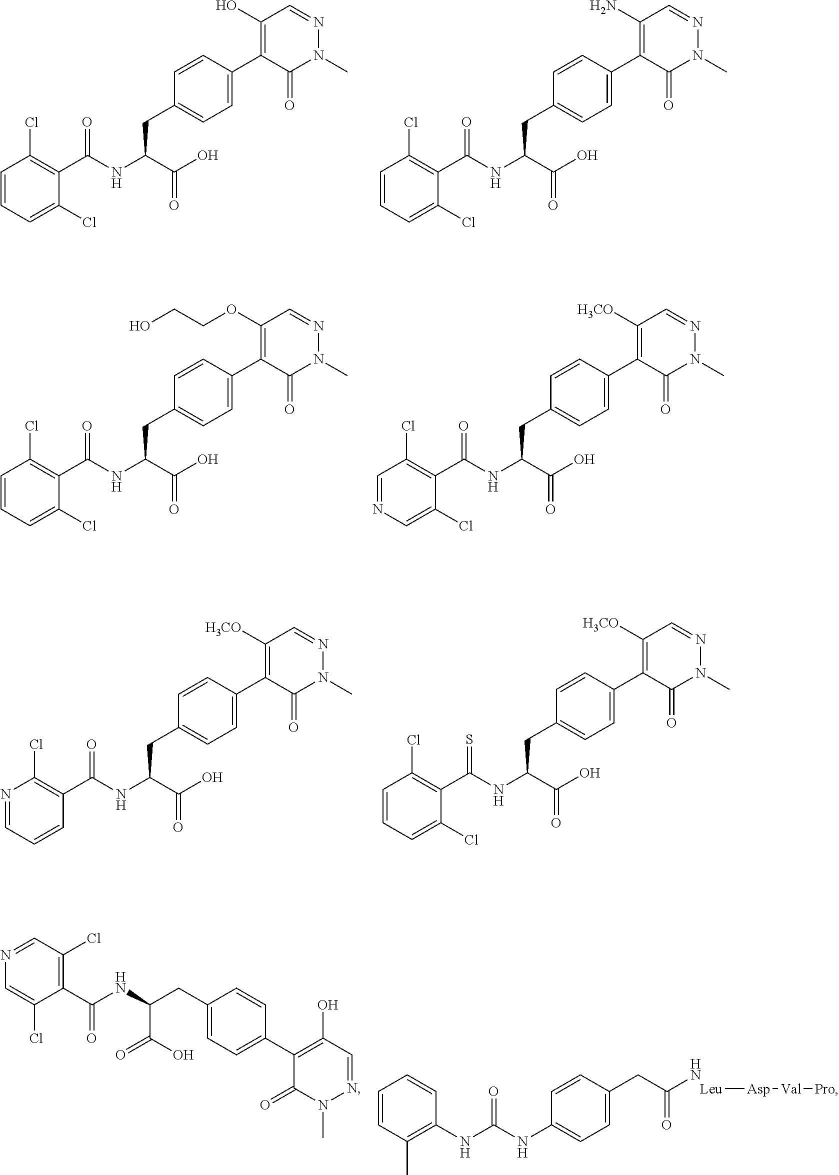

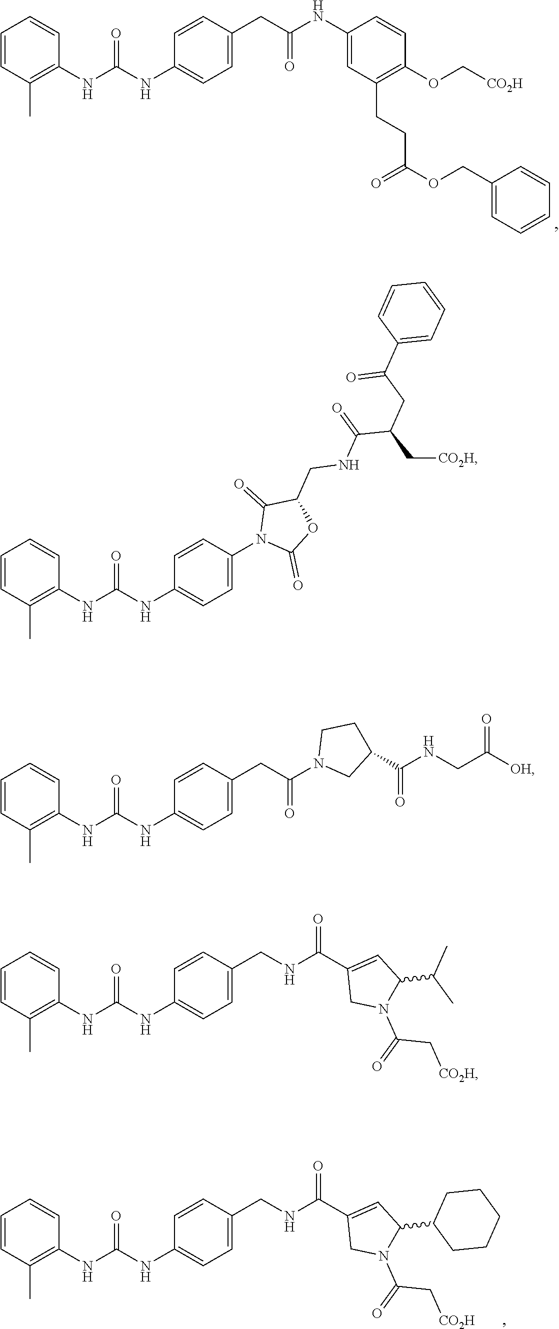 [SMILES: Cc1ccccc1NC(=O)Nc1ccc(CC(=O)Nc2ccc(OCC(=O)O)c(CCC(=O)OCc3ccccc3)c2)cc1 ; Cc1ccccc1NC(=O)Nc1ccc(N2C(=O)O[C@@H](CNC(=O)[C@H](CC(=O)O)CC(=O)c3ccccc3)C2=O)cc1 ; Cc1ccccc1NC(=O)Nc1ccc(CC(=O)N2CC[C@H](C(=O)NCC(=O)O)C2)cc1 ; Cc1ccccc1NC(=O)Nc1ccc(CNC(=O)C2=CC(C(C)C)N(C(=O)CC(=O)O)C2)cc1 ; Cc1ccccc1NC(=O)Nc1ccc(CNC(=O)C2=CC(C3CCCCC3)N(C(=O)CC(=O)O)C2)cc1]

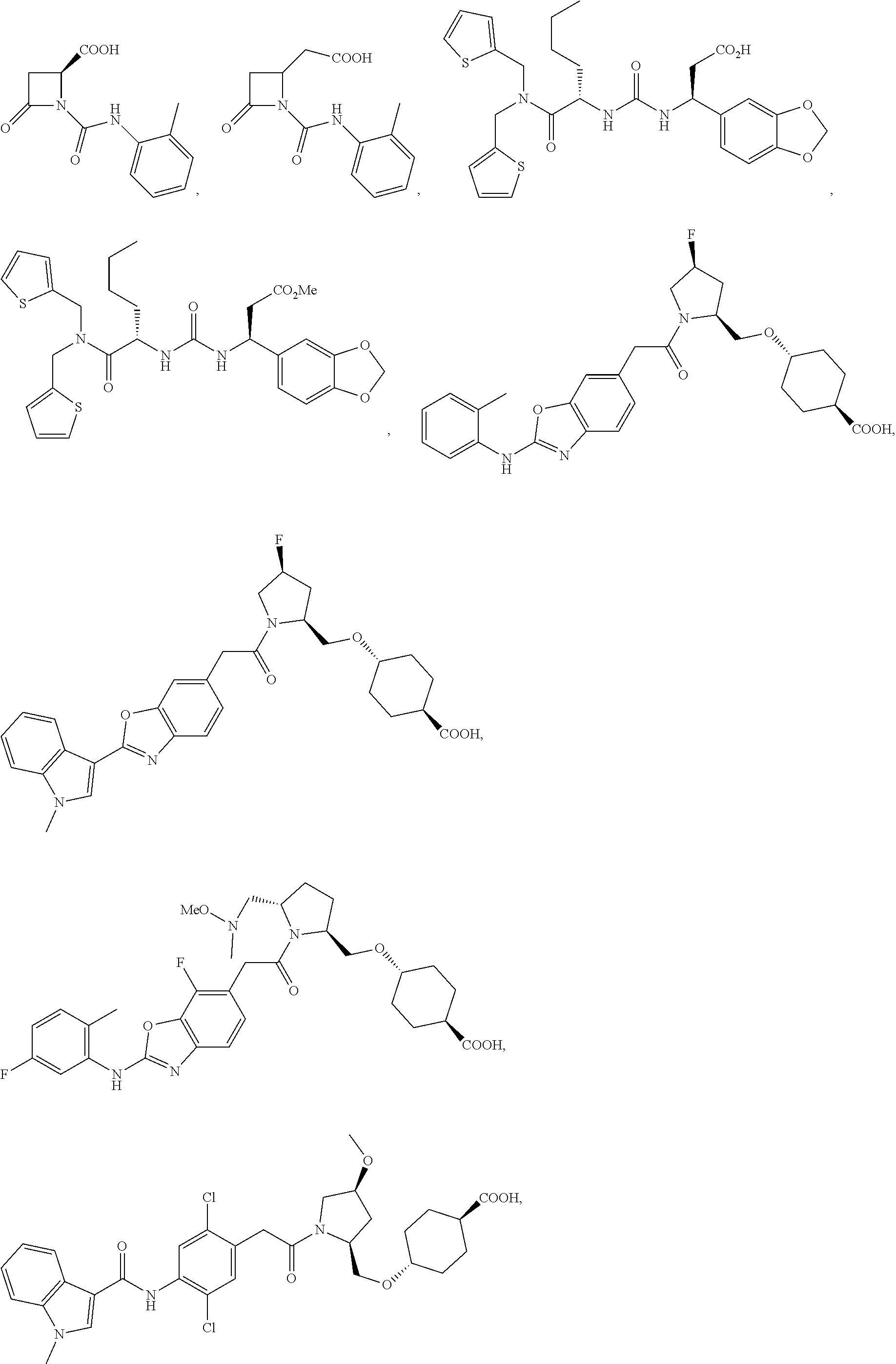

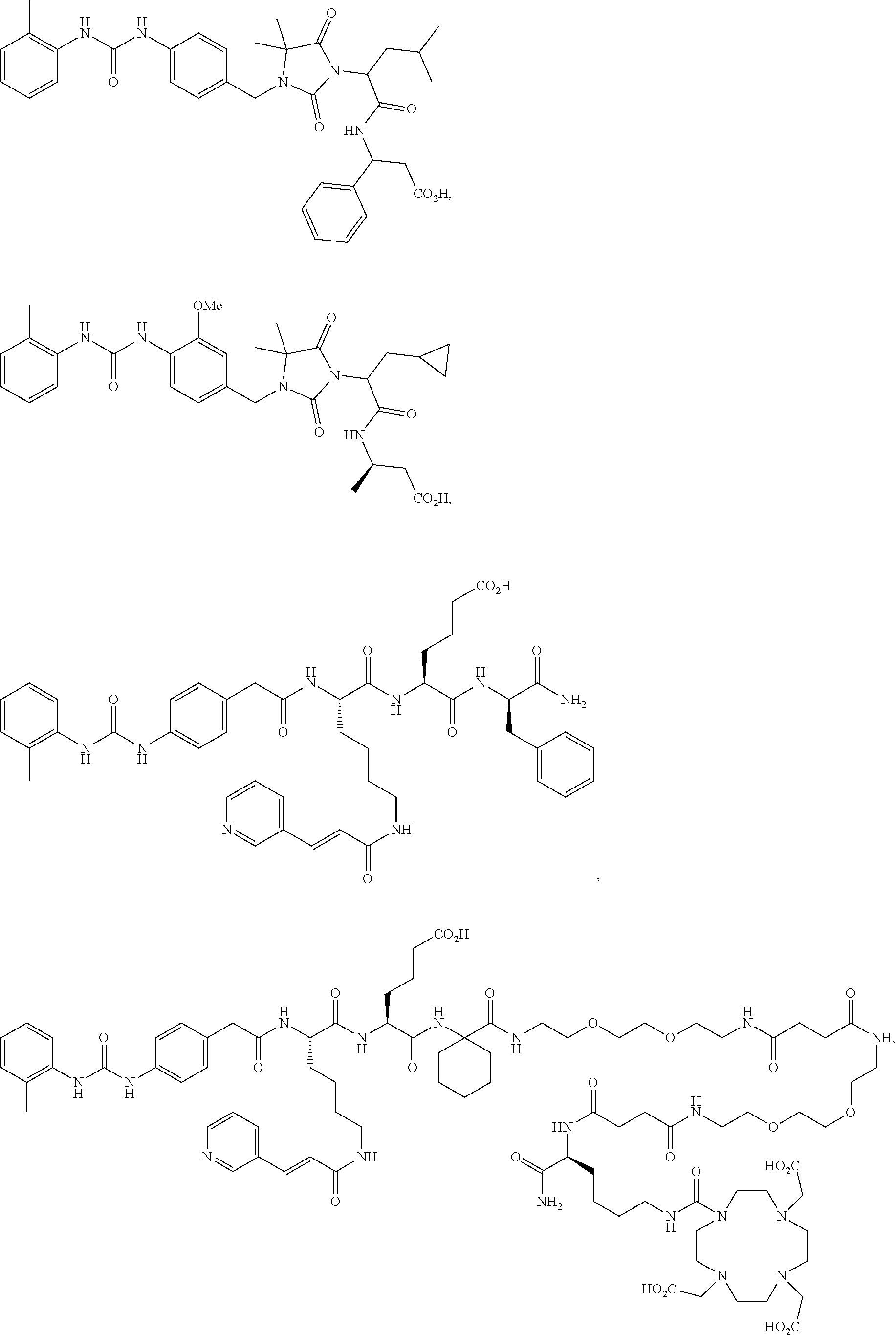

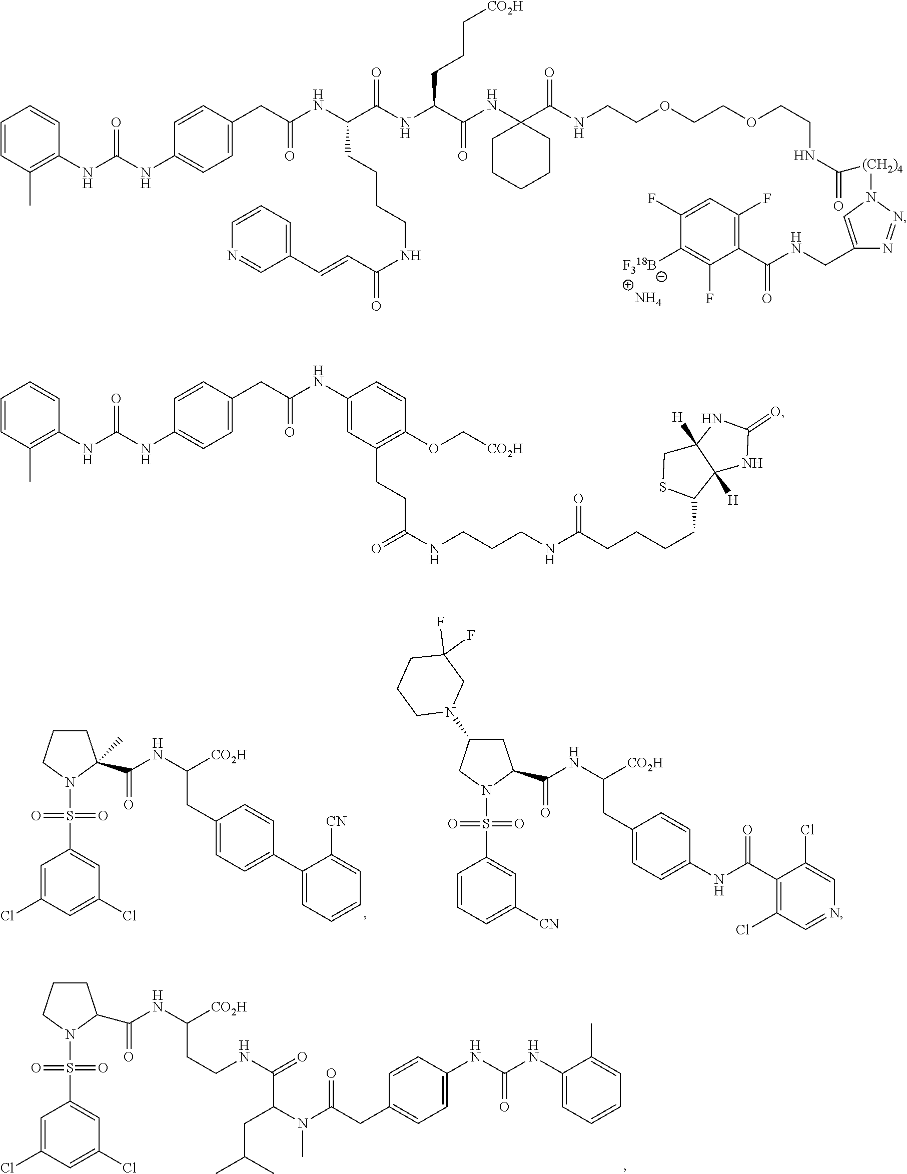

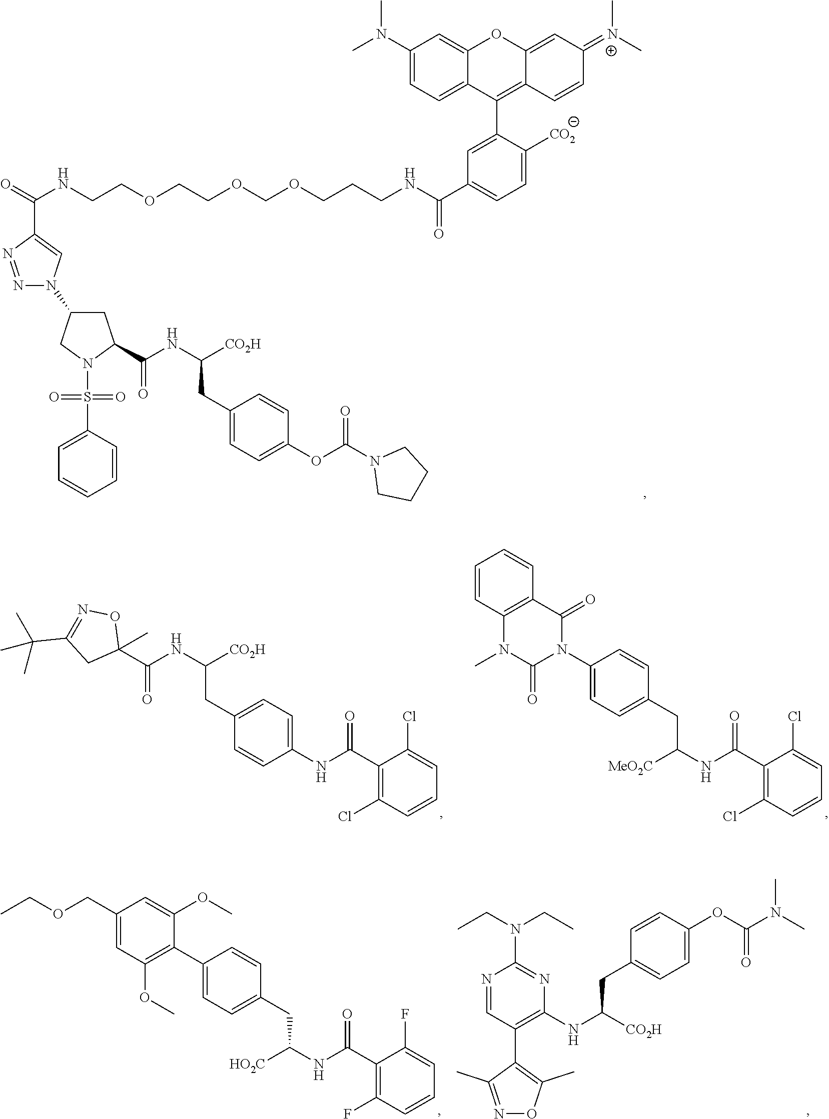

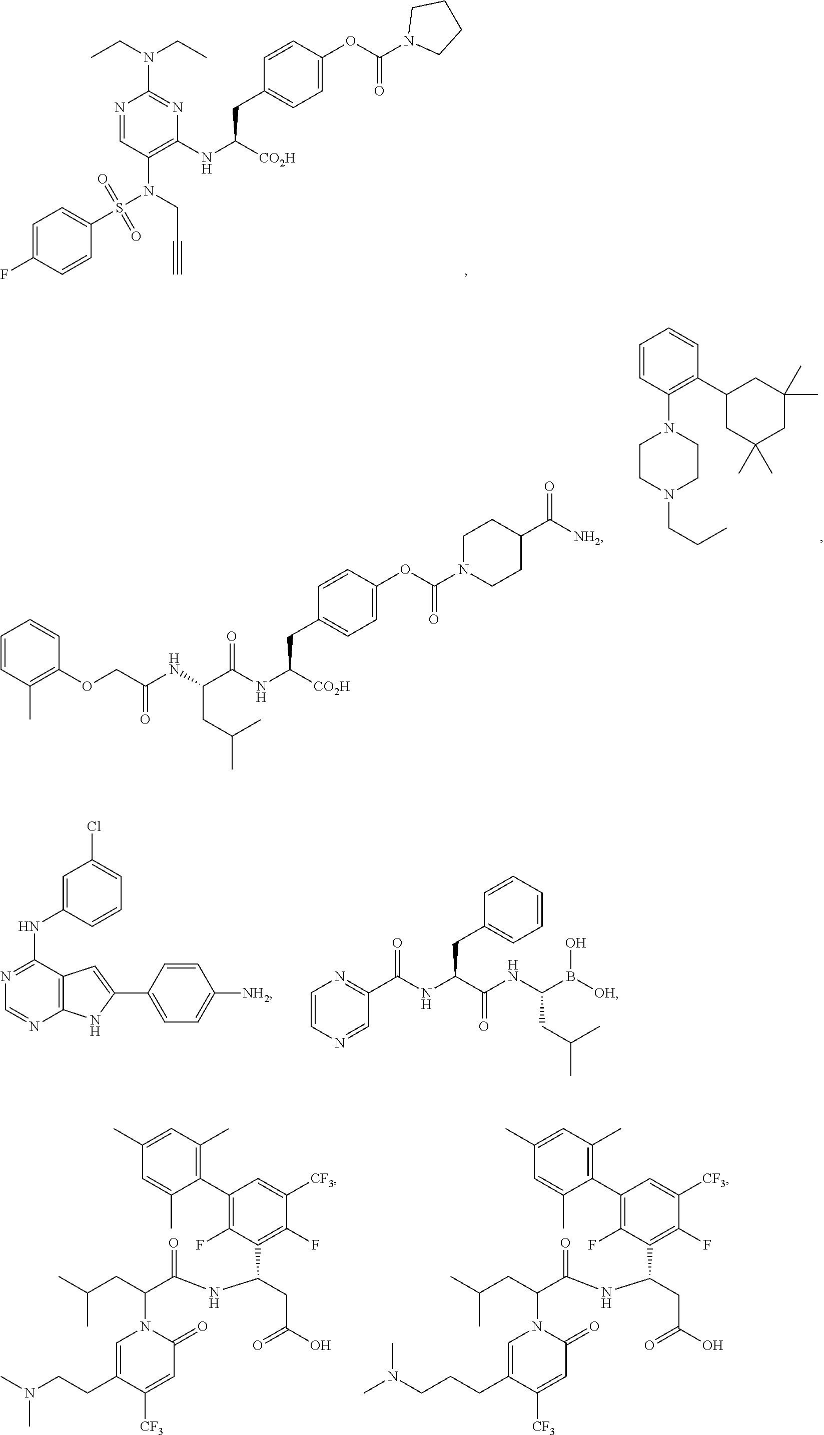

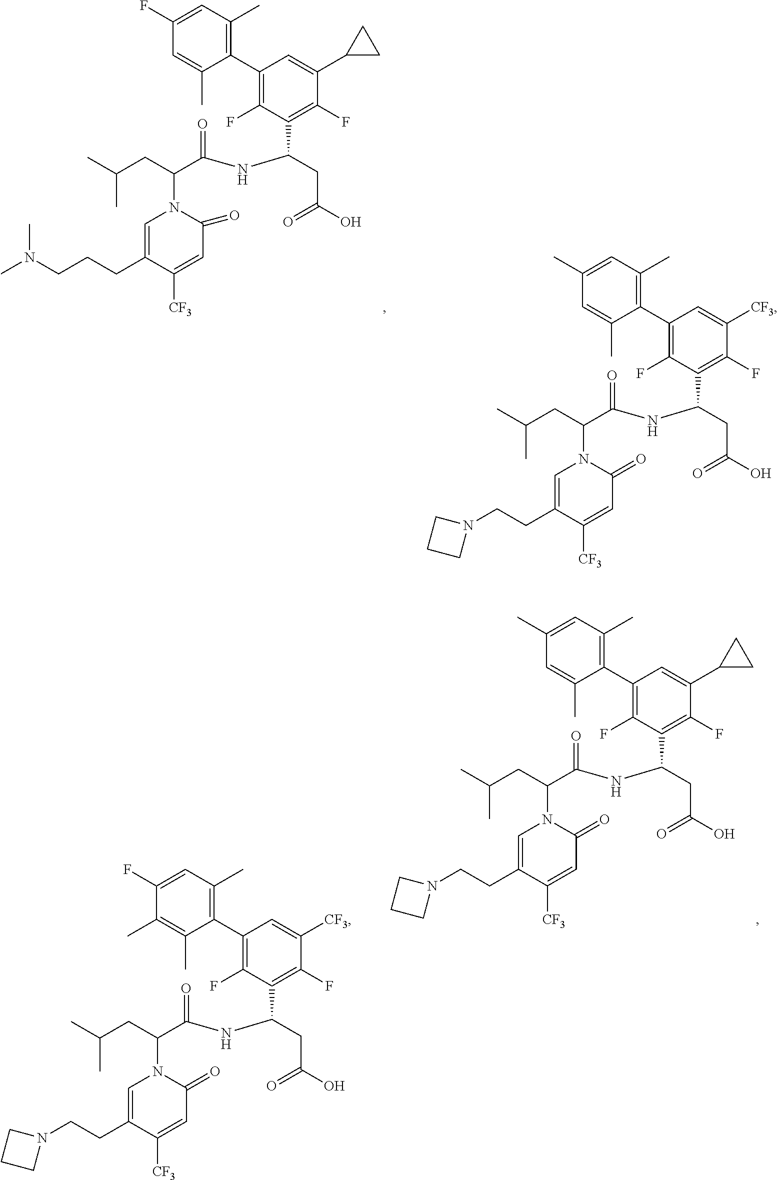 [SMILES: Cc1cc(F)cc(C)c1-c1cc(C2CC2)c(F)c([C@H](CC(=O)O)NC(=O)C(CC(C)C)n2cc(CCCN(C)C)c(C(F)(F)F)cc2=O)c1F ; Cc1cc(C)c(-c2cc(C(F)(F)F)c(F)c([C@H](CC(=O)O)NC(=O)C(CC(C)C)n3cc(CCN4CCC4)c(C(F)(F)F)cc3=O)c2F)c(C)c1 ; Cc1cc(F)c(C)c(C)c1-c1cc(C(F)(F)F)c(F)c([C@H](CC(=O)O)NC(=O)C(CC(C)C)n2cc(CCN3CCC3)c(C(F)(F)F)cc2=O)c1F ; Cc1cc(C)c(-c2cc(C3CC3)c(F)c([C@H](CC(=O)O)NC(=O)C(CC(C)C)n3cc(CCN4CCC4)c(C(F)(F)F)cc3=O)c2F)c(C)c1]

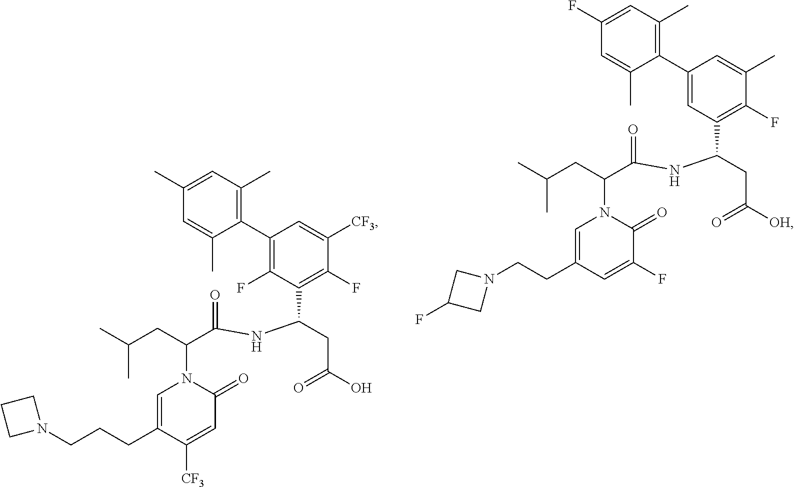 [SMILES: Cc1cc(C)c(-c2cc(C(F)(F)F)c(F)c([C@H](CC(=O)O)NC(=O)C(CC(C)C)n3cc(CCCN4CCC4)c(C(F)(F)F)cc3=O)c2F)c(C)c1 ; Cc1cc(-c2c(C)cc(F)cc2C)cc([C@H](CC(=O)O)NC(=O)C(CC(C)C)n2cc(CCN3CC(F)C3)cc(F)c2=O)c1F]

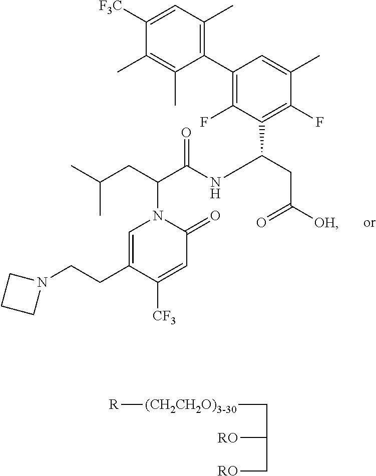

wherein each instance of R is

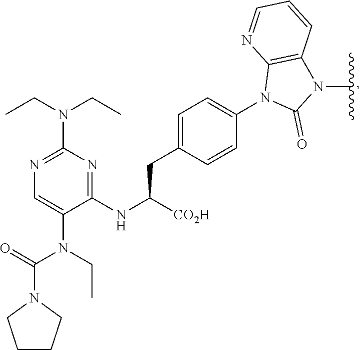 [SMILES: *n1c(=O)n(-c2ccc(C[C@H](Nc3nc(N(CC)CC)ncc3N(CC)C(=O)N3CCCC3)C(=O)O)cc2)c2ncccc21]

In certain embodiments, an α₄β_{1/7} integrin receptor ligand is

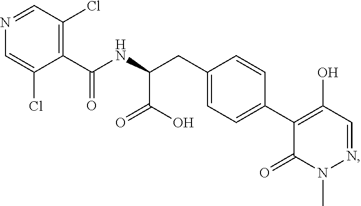 [SMILES: Cn1ncc(O)c(-c2ccc(C[C@H](NC(=O)c3c(Cl)cncc3Cl)C(=O)O)cc2)c1=O]

or a derivative thereof.

In some embodiments, an α₄₁in integrin receptor ligand is an anti-α₄β_{1/7} integrin receptor antibody. In certain embodiments, an α₄β_{1/7} integrin receptor ligand is an anti-α₄β_{1/7} integrin receptor antibody fragment, or an anti-α₄β_{1/7} integrin receptor antibody variant. An “anti-α₄β_{1/7} integrin receptor antibody” refers to an immune system protein that recognizes, binds to, or otherwise interacts with an α₄β_{1/7} integrin receptor.

In certain embodiments, an α₄β_{1/7} integrin receptor ligand is conjugated (e.g., linked, connected, attached, associated with) to and one or more agent moieties. In certain embodiments, the agent moiety is a therapeutic, prophylactic, diagnostic, or imaging agent. In certain embodiments, the agent is a small molecule or oligomeric compound. In certain embodiments, the agent moiety is a protein, a peptide, an antibody, an oligonucleotide, a small molecule, a large molecule, or a combination thereof.

In some embodiments, more than one α₄β_{1/7} integrin receptor ligand is conjugated to an agent moiety. In some embodiments, at least two α₄β_{1/7} integrin receptor ligands (e.g., 2, 3, 4, 5, 6, 7, 8, 9, 10, or more α₄β₁₇ integrin receptor ligands) are conjugated to an agent moiety. In some embodiments, two α₄β_{1/7} integrin receptor ligands are conjugated to an agent moiety. In some embodiments, three α₄β_{1/7} integrin receptor ligands are conjugated to an agent moiety. In some embodiments, four α₄β_{1/7} integrin receptor ligands are conjugated to an agent moiety. In some embodiments, five α₄β_{1/7} integrin receptor ligands are conjugated to an agent moiety. In some embodiments, more than five α₄β_{1/7} integrin receptor ligands are conjugated to an agent moiety. In some embodiments, at least 1 to about 5 α₄β_{1/7} integrin receptor ligands are conjugated to an agent moiety. In some embodiments, at least 1 to about 4 α₄β_{1/7} integrin receptor ligands are conjugated to an agent moiety. In some embodiments, at least 1 to about 3 α₄β_{1/7} integrin receptor ligands are conjugated to an agent moiety. In some embodiments, at least 1 to about 2 α₄β_{1/7} integrin receptor ligands are conjugated to an agent moiety.

When an agent moiety is conjugated to multiple α₄β_{1/7} integrin receptor ligands, all of the α₄β_{1/7} integrin receptor ligands may be conjugated at or near the same position on the agent moiety, or the α₄β_{1/7} integrin receptor ligands may be conjugated to multiple different positions on the agent moiety.

In some embodiments, an oligonucleotide is conjugated (e.g., connected, attached, associated with) to an α₄β_{1/7} integrin receptor ligand through either a 5′ end and/or a 3′ end of the oligonucleotide, or at an internal position in an oligonucleotide (i.e., at a nucleotide on the oligonucleotide other than the 5′ or 3′ nucleotide). In some embodiments, an oligonucleotide is conjugated to an α₄β_{1/7} integrin receptor ligand through the 5′ end of the oligonucleotide. In some embodiments, an oligonucleotide is conjugated to an α₄β_{1/7} integrin receptor ligand through the 3′ end of the oligonucleotide. In some embodiments, an oligonucleotide is conjugated to α₄β_{1/7} integrin receptor ligands through both the 5′ end and the 3′ end of the oligonucleotide. In some embodiments, an oligonucleotide is conjugated to an α₄β_{1/7} integrin receptor ligand at an internal position within the oligonucleotide (e.g., in an “internally-modified oligonucleotide”).

In some embodiments, an oligonucleotide is conjugated to more than one α₄β_{1/7} integrin receptor ligand. In some embodiments, an oligonucleotide is conjugated to at least two α₄β_{1/7} integrin receptor ligands (e.g., 2, 3, 4, 5, 6, 7, 8, 9, 10, or more α₄β_{1/7} integrin receptor ligands). In some embodiments, an oligonucleotide is conjugated to two α₄β_{1/7} integrin receptor ligands. In some embodiments, an oligonucleotide is conjugated to three α₄β_{1/7} integrin receptor ligands. In some embodiments, an oligonucleotide is conjugated to four α₄β_{1/7} integrin receptor ligands. In some embodiments, an oligonucleotide is conjugated to five α₄β_{1/7} integrin receptor ligands. In some embodiments, an oligonucleotide is conjugated to more than five α₄β_{1/7} integrin receptor ligands. In some embodiments, an oligonucleotide is conjugated to at least 1 to about 5 α₄β_{1/7} integrin receptor ligands. In some embodiments, an oligonucleotide is conjugated to at least 1 to about 4 α₄β_{1/7} integrin receptor ligands. In some embodiments, an oligonucleotide is conjugated to at least 1 to about 3 α₄β_{1/7} integrin receptor ligands. In some embodiments, an oligonucleotide is conjugated to at least 1 to about 2 α₄β_{1/7} integrin receptor ligands.

When an oligonucleotide is conjugated to multiple α₄β₁₇ integrin receptor ligands, all of the α₄β_{1/7} integrin receptor ligands may be conjugated at or near the same position on the oligonucleotide, or the α₄β_{1/7} integrin receptor ligands may be conjugated to multiple different positions on the oligonucleotide. In some embodiments, multiple α₄β_{1/7} integrin receptor ligands (i.e., two, three, four, five, or more α₄β_{1/7} integrin receptor ligands) are conjugated at the 5′ end of the oligonucleotide. In some embodiments, multiple α₄β_{1/7} integrin receptor ligands (i.e., two, three, four, five, or more α₄β_{1/7} integrin receptor ligands) are conjugated at the 3′ end of the oligonucleotide. In some embodiments, multiple α₄β_{1/7} integrin receptor ligands (i.e., two, three, four, five, or more α₄β₁₇ integrin receptor ligands) are conjugated at one or more internal positions of the oligonucleotide. In some embodiments, an oligonucleotide is conjugated to one or more α₄β_{1/7} integrin receptor ligands at the 5′ end of the oligonucleotide and/or one or more α₄β_{1/7} integrin receptor ligands at the 3′ end of the oligonucleotide and/or one or more α₄β_{1/7} integrin receptor ligands at an internal position, or multiple internal positions, of the oligonucleotide. Linkers

In certain embodiments, conjugates of the compound formulae described herein are provided. In certain embodiments, the conjugates comprise an α₄β_{1/7} integrin receptor ligand covalently coupled to an agent moiety. In certain embodiments, the conjugates provided herein comprise one or more linker moieties. In certain embodiments, the one or more linker moieties link an α₄β_{1/7} integrin receptor ligand to an agent moiety. In certain embodiments, the agent moiety is a protein, peptide, antibody, nucleic acid, small molecule, large molecule, therapeutic, prophylactic, diagnostic, or imaging agent. In some embodiments, a compound is conjugated to an oligonucleotide. In certain embodiment an α₄β_{1/7} integrin receptor ligand is conjugated to an oligonucleotide. In certain embodiments, a compound comprises one or more α₄β_{1/7} integrin receptor ligands, one or more linker moieties, and one or more agent moieties, wherein the α₄β_{1/7} integrin receptor ligands are conjugated (e.g., linked, connected, attached, associated with) to the one of more agent moieties through one or more linker moieties.

Conjugates as disclosed herein can be manufactured using any available method. When associating compounds provided herein with agent moieties (e.g., an α₄β_{1/7} integrin receptor ligand with an oligonucleotide), the moieties may be linked directly or indirectly (e.g., through a linker moiety; that is, the linker is covalently bonded to each of the oligonucleotide and the α₄β_{1/7} integrin receptor ligand; in some formulae herein “-Lₙ-” wherein n is a number (e.g., L₁, L₂, L₃, L₄, L_{1A}, L_{2A}, L_{3A}, L_{4A})). For example, the oligonucleotide and α₄β_{1/7} integrin receptor ligand may be directly associated with one another, e.g., by one or more covalent bonds, or may be associated by means of one or more linkers. A “linker” refers to any chemical moiety (e.g., a combination of atoms having appropriate valency according to known chemistry principles) used to conjugate two components of the compounds provided herein (e.g., an α₄β₁₇ integrin receptor ligand and an oligonucleotide) to one another. Each of the two components may be connected to any portion of any of the linkers provided herein. In some embodiments, one component of the compounds provided herein (e.g., an α₄β_{1/7} integrin receptor ligand or an oligonucleotide) is connected by a bond to one end of a linker, and the other component is connected by a bond to the other end of the linker. In some embodiments, one or both components of the compounds provided herein may be connected by a bond to an internal position within any of the linkers described herein. For example, in the context of an “alkyl linker,” an α₄β_{1/7} integrin receptor ligand may be joined by a bond to a carbon at one end of the alkyl linker, and an oligonucleotide may be joined by a bond to a carbon at the other end of the alkyl linker. In some embodiments, a linker is a bond (including, e.g., phosphodiester and phosphorothioate bonds). In some embodiments, a linker is an optionally substituted alkyl linker (i.e., an alkyl chain is used to join two moieties, which may each be conjugated to opposite ends of the alkyl linker, or one or both moieties may be conjugated to an internal carbon on the alkyl linker). In some embodiments, a linker is an optionally substituted polyethylene glycol (PEG) linker (i.e., a PEG chain is used to join two moieties, which may each be conjugated to opposite ends of the PEG linker, or one or both moieties may be conjugated to an internal position on the PEG linker). In some embodiments, a linker is an optionally substituted heteroalkyl linker (i.e., a heteroalkyl chain is used to join two moieties, which may each be conjugated to opposite ends of the heteroalkyl linker, or one or both moieties may be conjugated to an internal position on the heteroalkyl linker). In some embodiments, a linker is an optionally substituted heteroaryl linker (i.e., a heteroaryl group is used to join two moieties, which may each be conjugated to any position on the heteroaryl group).

In certain embodiments, the compounds provided herein comprise one or more linking groups. In certain embodiments, each of L₁, L₂, L₃, and L₄ comprises a linking group. In certain embodiments, each of L₁, L₂, L₃, and L₄ comprises a linking group. In certain embodiments, each of L₁, L₂, L₃, L₄, L_{1A}, L_{2A}, L_{3A}, and L_{4A}, comprises a linking group. In certain embodiments, a linking group is covalently bound to an α₄β_{1/7} integrin receptor ligand. In certain embodiments, a linking group is covalently bound to an oligonucleotide. In certain embodiments, a linking group is covalently bound to a cleavable moiety. In certain embodiments, a linking group comprises a cleavable bond. In certain embodiments, a linking group does not comprise a cleavable moiety. In certain embodiments, a linking group comprises a covalent attachment to a solid support. In certain embodiments, a linking group includes multiple positions for attachment of α₄β_{1/7} integrin receptor ligands.

In certain embodiments, a linking group comprises a chain structure, such as a hydrocarbyl chain, or an oligomer of repeating units or combination of such repeating units. In certain embodiments, a linking group comprises 1 to 50 repeating units, 1 to 40 repeating units, 1 to 25 repeating units, 1 to 20 repeating units, 1 to 15 repeating units, 1 to 10 repeating units, or 1 to 5 repeating units. In certain embodiments, a linking group is 1 to 50 atoms long, 1 to 40 atoms long, 1 to 25 atoms long, 1 to 20 atoms long, 1 to 15 atoms long, 1 to 10 atoms long, or 1 to 5 atoms long.

In certain embodiments, a linking group contains carbon atoms. In certain embodiments, a linking group contains heteroatoms (e.g., nitrogen, oxygen, sulfur, etc.). In certain embodiments, a linking group forms amide linkages, ester linkages, or disulfide linkages. In certain embodiments, a linking group forms hydrazone linkages, oxime linkages, imine linkages, guanidine linkages, urea linkages, carbamate linkages, unsaturated alkyl linkages, sulfonamide linkages or 4-8 membered hetero cyclic linkages. In certain embodiments, a linking group comprises one or more groups selected from alkyl, amino, oxo, amide, disulfide, polyethylene glycol, ether, thioether, and hydroxylamino. In certain embodiments, a linking group comprises at least one phosphorus group. In certain embodiments, a linking group comprises at least one phosphate group. In certain embodiments, a linking group includes at least one neutral linking group. In certain embodiments, a linking group is substituted with various substituents including, but not limited to, hydrogen atoms, alkyl, alkenyl, alkynyl, amino, alkylamino, dialkylamino, trialkylamino, hydroxyl, alkoxy, halogen, aryl, heterocyclic, aromatic heterocyclic, cyano, amide, carbamoyl, carboxylic acid, ester, thioether, alkylthioether, thiol, and ureido groups. As would be appreciated by one of skill in this art, each of these groups may in turn be substituted.

In certain embodiments, a linking group includes, but is not limited to, substituted or unsubstituted C1-C10 alkyl, substituted or unsubstituted C2-C10 alkenyl, or substituted or unsubstituted C2-C10 alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl, and alkynyl. In certain embodiments, a linking group is an aliphatic or heteroaliphatic. For example, the linking group can be a polyalkyl linking group. The linking group can be a polyether linking group. The linking group can be a polyethylene linking group, such as PEG.

In certain embodiments, the linking group is a short peptide chain. In certain embodiments, a linking group comprises 1 to 40 amino acids, 1 to 25 amino acids, 1 to 20 amino acids, 1 to 15 amino acids, 1 to 10 amino acids, or 1 to 5 amino acids.

In certain embodiments, a linking group comprises linker-nucleosides. In certain embodiments, a linking group comprises 1 to 40 linker-nucleosides, 1 to 25 linker-nucleosides, 1 to 20 linker-nucleosides, 1 to 15 linker-nucleosides, 1 to 10 linker-nucleosides, or 1 to 5 linker-nucleosides. In certain embodiments, such linker-nucleosides may be modified or unmodified nucleosides. It is typically desirable for linker-nucleosides to be cleaved from the compound after it reaches a target tissue. Accordingly, linker-nucleosides herein can be linked to one another and to the remainder of the compound through cleavable bonds. Herein, linker-nucleosides are not considered to be part of an oligonucleotide payload. Accordingly, in embodiments in which a compound comprises an oligonucleotide consisting of a specified number or range of linked nucleosides and/or a specified percent complementarity to a reference nucleic acid, and the compound also comprises an α₄β_{1/7} integrin receptor ligand comprising a linking group comprising linker-nucleosides, those linker-nucleosides are not counted toward the length of the oligonucleotide and are not used in determining the percent complementarity of the oligonucleotide for the reference nucleic acid.

In certain embodiments, the linking group includes a protein binding group. In certain embodiments, the protein binding group is a lipid such as, for example, including but not limited to cholesterol, cholic acid, adamantane acetic acid, 1-pyrene butyric acid, dihydrotestosterone, 1,3-Bis- O(hexadecyl)glycerol, geranyloxyhexyl group, hexadecylglycerol, borneol, menthol, 1,3-propanediol, heptadecyl group, palmitic acid, myristic acid, 03-(oleoyl)lithocholic acid, 03-(oleoyl)cholenic acid, dimethoxytrityl, or phenoxazine), a vitamin (e.g., folate, vitamin A, vitamin E, biotin, pyridoxal), a peptide, a carbohydrate (e.g., monosaccharide, disaccharide, trisaccharide, tetrasaccharide, oligosaccharide, polysaccharide), an endosomolytic component, a steroid (e.g., uvaol, hecigenin, diosgenin), a terpene (e.g., triterpene, e.g., sarsasapogenin, friedelin, epifriedelanol derivatized lithocholic acid), or a cationic lipid. In certain embodiments, the protein binding group is a C16 to C22 long chain saturated or unsaturated fatty acid, cholesterol, cholic acid, vitamin E, adamantane or 1-pentafluoropropyl.

In certain embodiments, a linking group includes, but is not limited to, pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA).

In certain embodiments, a linking group includes, without limitation, those linking groups described in the following references: U.S. Pat. Nos. 5,994,517; 6,300,319; 6,660,720; 6,906,182; 7,262,177; 7,491,805; 8,106,022; 7,723,509; 9,127,276; U.S. 2006/0148740; U.S. 2011/0123520; WO 2013/033230; WO 2012/037254, Biessen et al., J. Med. Chem. 1995, 38, 1846-1852; Lee et al., Bioorganic & Medicinal Chemistry 2011,19, 2494-2500; Rensen et al., J. Biol. Chem. 2001, 276, 37577-37584; Rensen et al., J. Med. Chem. 2004, 47, 5798-5808; Sliedregt et al., J. Med. Chem. 1999, 42, 609-618; Valentijn et al., Tetrahedron, 1997, 53, 759-770; Lee, Carbohydr. Res. 1978, 67, 509-514; Connolly et al., J. Biol. Chem. 1982, 257, 939-945; Pavia et al., Int. J. Pep. Protein Res. 1983, 22, 539-548; Lee et al., Biochem. 1984, 23, 4255-4261; Lee et al., Glycoconjugate J. 1987, 4, 317-328; Toyokuni et al., Tetrahedron Lett. 1990, 31, 2673-2676; Biessen et al., J. Med. Chem. 1995, 38, 1538-1546; Valentijn et al., Tetrahedron, 1997, 53, 759-770; Kim et al., Tetrahedron Lett. 1997, 38, 3487-3490; Lee et al., Bioconjug. Chem. 1997, 8, 762-765; Kato et al., Glycobiol. 2001, 11, 821-829; Rensen et al., J. Biol. Chem. 2001, 276, 37577-37584; Lee et al., Methods Enzymol. 2003, 362, 38-43; Westerlind et al., Glycoconj. J. 2004, 21, 227-241; Lee et al., Bioorg. Med. Chem. Lett. 2006, 16(19), 5132-5135; Maierhofer et al., Bioorg. Med. Chem. 2007, 15, 7661-7676; Khorev et al., Bioorg. Med. Chem. 2008, 16, 5216-5231; Lee et al., Bioorg. Med. Chem. 2011, 19, 2494-2500; Kornilova et al., Analyt. Biochem. 2012, 425, 43-46; Pujol et al., Angew. Chemie Int. Ed. Engl. 2012, 51, 7445-7448; Biessen et al., J. Med. Chem. 1995, 38, 1846-1852; Sliedregt et al., J. Med. Chem. 1999, 42, 609-618; Rensen et al., J. Med. Chem. 2004, 47, 5798-5808; Rensen et al., Arterioscler. Thromh. Vase. Biol. 2006, 26, 169-175; van Rossenberg et al., Gene Ther. 2004, 11, 457-464; Sato et al., J. Am. Chem. Soc. 2004, 126, 14013-14022; Lee et al., J. Org. Chem. 2012, 77, 7564-7571; Biessen et al., FASEB J. 2000, 14, 1784-1792; Rajur et al., Bioconjug. Chem. 1997, 8, 935-940; Duff et al., Methods Enzymol. 2000, 313, 297-321; Maier et al., Bioconjug. Chem. 2003, 14, 18-29; Jayaprakash et al., Org. Lett. 2010, 12, 5410-5413; Manoharan, Antisense Nucleic Acid Drug Dev. 2002, 12, 103-128; Merwin et al., Bioconjug. Chem. 1994, 5, 612-620; Tomiya et al., Bioorg. Med. Chem., 2013, 21, 5275-5281; International Applications WO 1998/013381; WO 2011/038356; WO 1997/046098; WO 2008/098788; WO 2004/101619; WO 2012/037254; WO 2011/120053; WO 2011/100131; WO 2011/163121; WO 2012/177947; WO 2013/033230; WO 2013/075035; WO 2012/083185; WO 2012/083046; WO 2009/082607; WO 2009/134487; WO 2010/144740; WO 2010/148013; WO 1997/020563; WO 2010/088537; WO 2002/043771; WO 2010/129709; WO 2012/068187; WO 2009/126933; WO 2004/024757; WO 2010/054406; WO 2012/089352; WO 2012/089602; WO 2013/166121; WO 2013/165816; U.S. Pat. Nos. 4,751,219; 7,582,744; 8,552,163; 8,137,695; 6,908,903; 6,383,812; 7,262,177; 6,525,031; 5,994,517; 6,660,720; 6,300,319; 7,723,509; 8,106,022; 7,491,805; 7,491,805; 8,541,548; 8,344,125; 8,313,772; 8,349,308; 8,450,467; 8,501,930; 8,158,601; 7,262,177; 6,906,182; 6,620,916; 8,435,491; 8,404,862; 7,851,615; U.S. Patent Application Publications Nos. U.S. 2011/0097264; U.S. 2011/0097265; U.S. 2013/0004427; U.S. 2003/0119724; U.S. 2011/0207799; U.S. 2012/0035115; U.S. 2012/0230938; U.S. 2005/0164235; U.S. 2006/0183886; U.S. 2012/0136042; U.S. 2012/0095075; U.S. 2013/0109817; U.S. 2006/0148740; U.S. 2008/0206869; U.S. 2012/0165393; U.S. 2012/0101148; U.S. 2013/0121954; U.S. 2011/0123520; U.S. 2003/0077829; U.S. 2008/0108801; and U.S. 2009/0203132; each of which is incorporated herein by reference.

In certain embodiments, L₁, L₂, L₃, and L₄ (or L₁, L₂, L₃, L₄, L_{1A}, L_{2A}, L_{3A}, and L_{4A}) independently comprise or together comprise a structure selected from among:

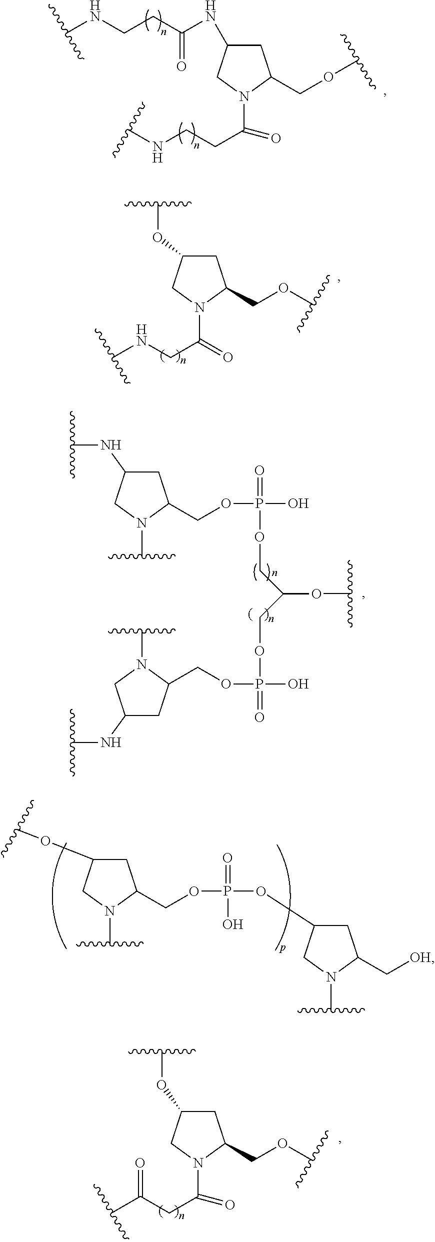

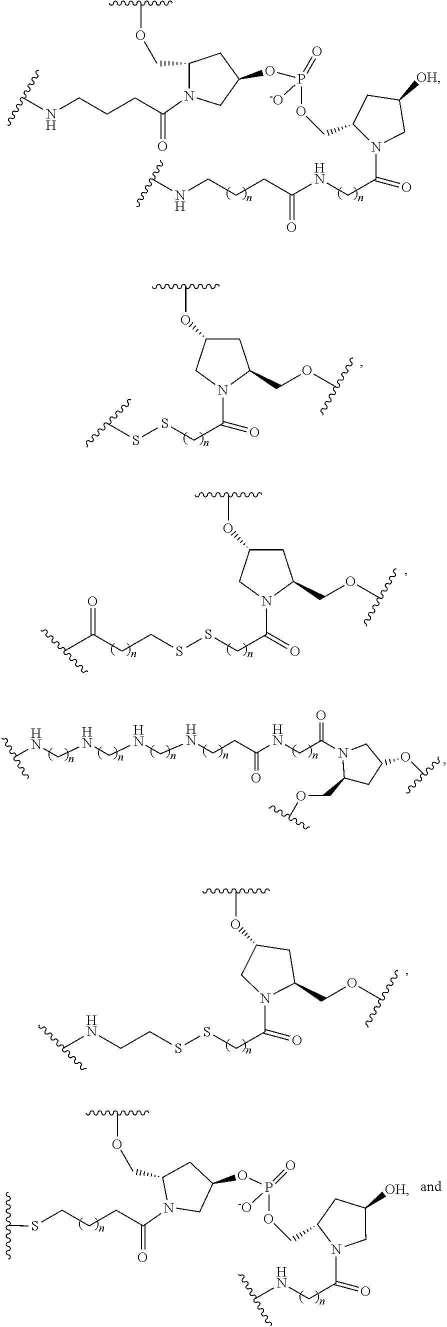

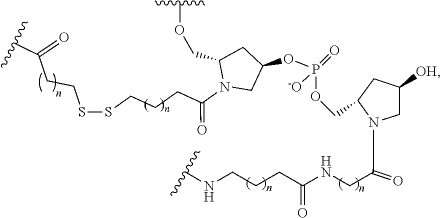

wherein each n is, independently, from 1 to 20; and p is from 1 to 6.

In certain embodiments, L₁, L₂, L₃, and L₄ (or L₁, L₂, L₃, L₄, L_{1A}, L_{2A}, L_{3A}, and L_{4A}) independently comprise or together comprise the structure selected from among:

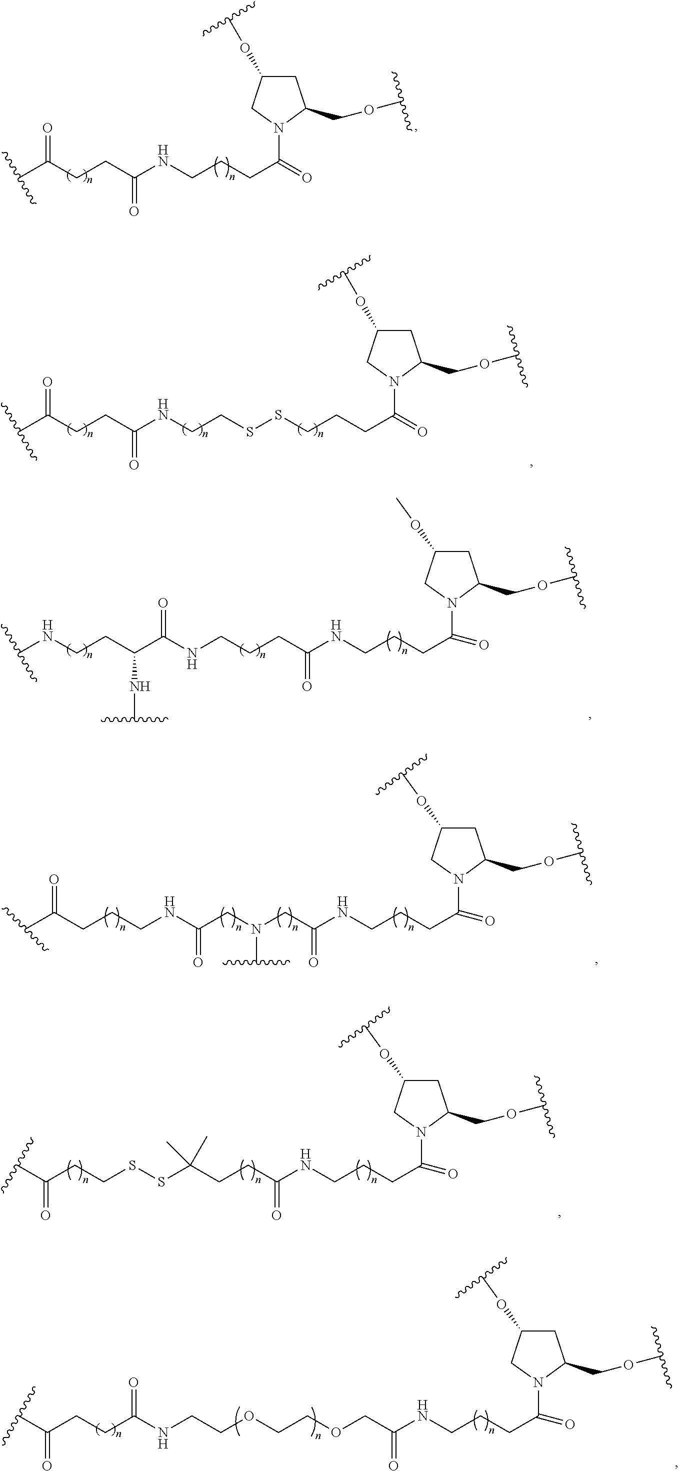

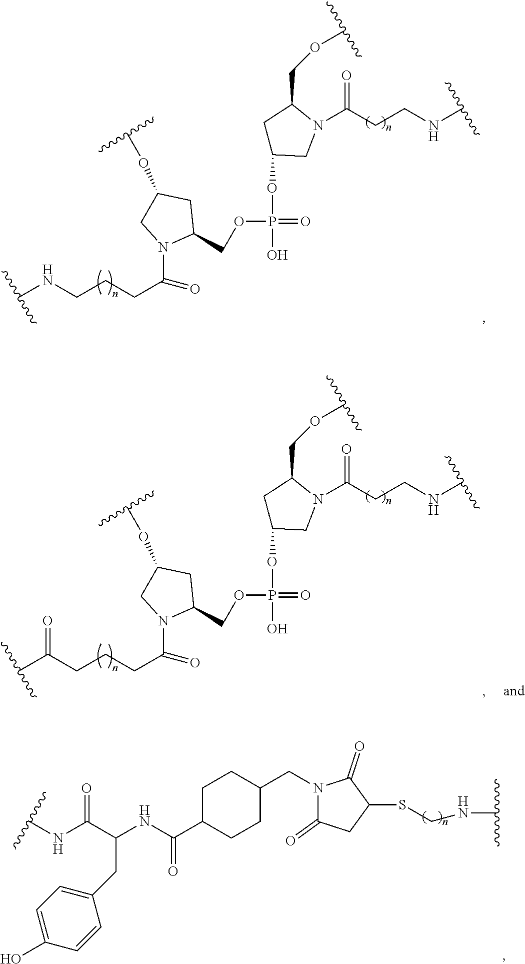

wherein each nis, independently, from 1to 20.

In certain embodiments, L₁, L₂, L₃, and L₄ (or L₁, L₂, L₃, L₄, L_{1A}, L_{2A}, L_{3A}, and L_{4A}) independently comprise or together comprise the structure selected from among:

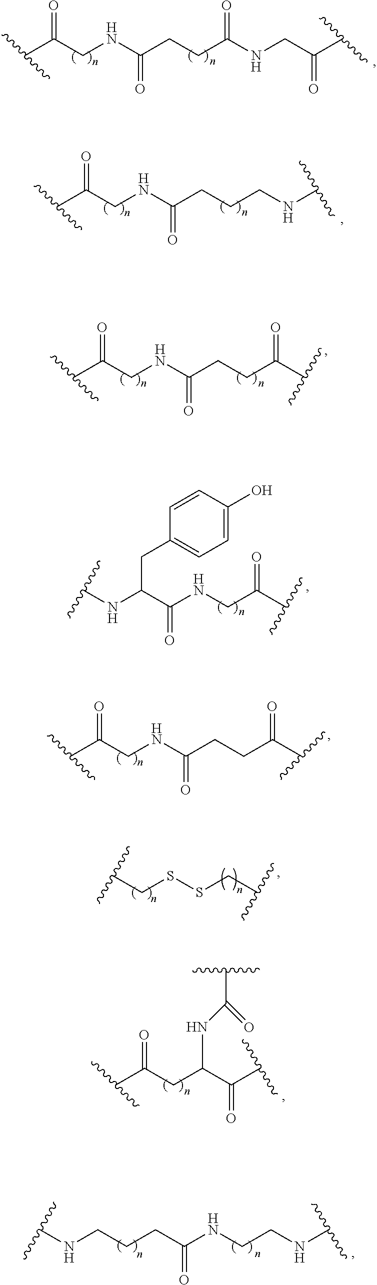

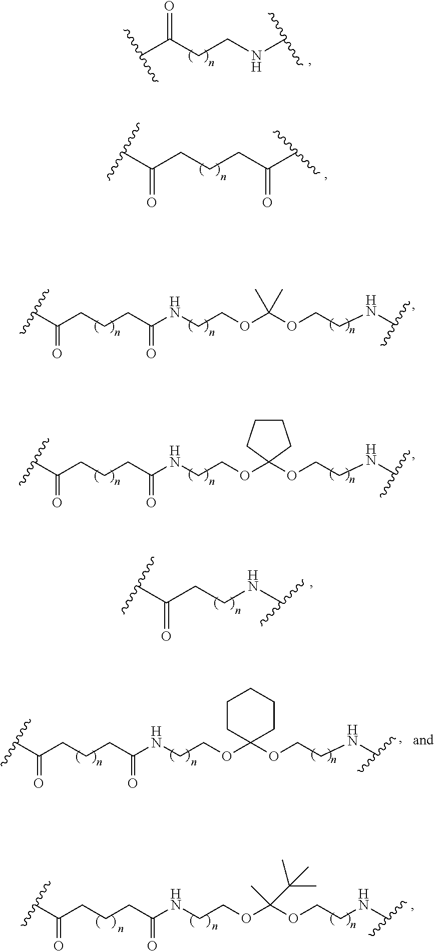

wherein each n is, independently, from 1 to 20.

In certain embodiments, L₁, L₂, L₃, and L₄ (or L₁, L₂, L₃, L₄, L_{1A}, L_{2A}, L_{3A}, and L_{4A}) independently comprise or together comprise the structure selected from among:

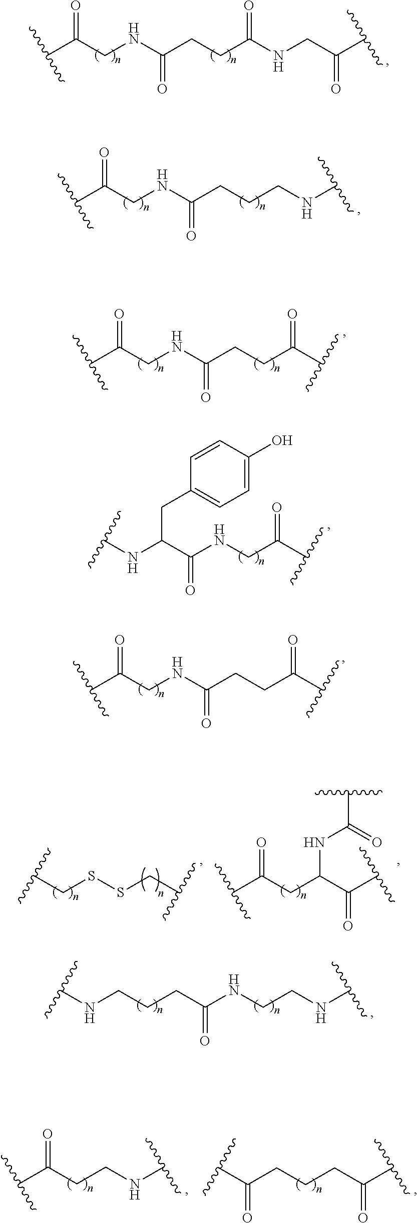

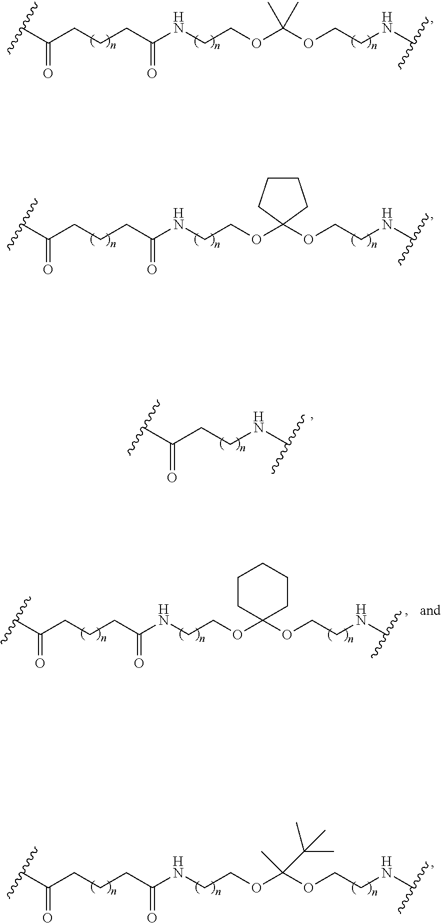

wherein each n is, independently, from 1 to 20.

In certain embodiments, L₁, L₂, L₃, and L₄ (or L₁, L₂, L₃, L₄, L_{1A}, L_{2A}, L_{3A}, and L_{4A}) independently comprise or together comprise the structure selected from among:

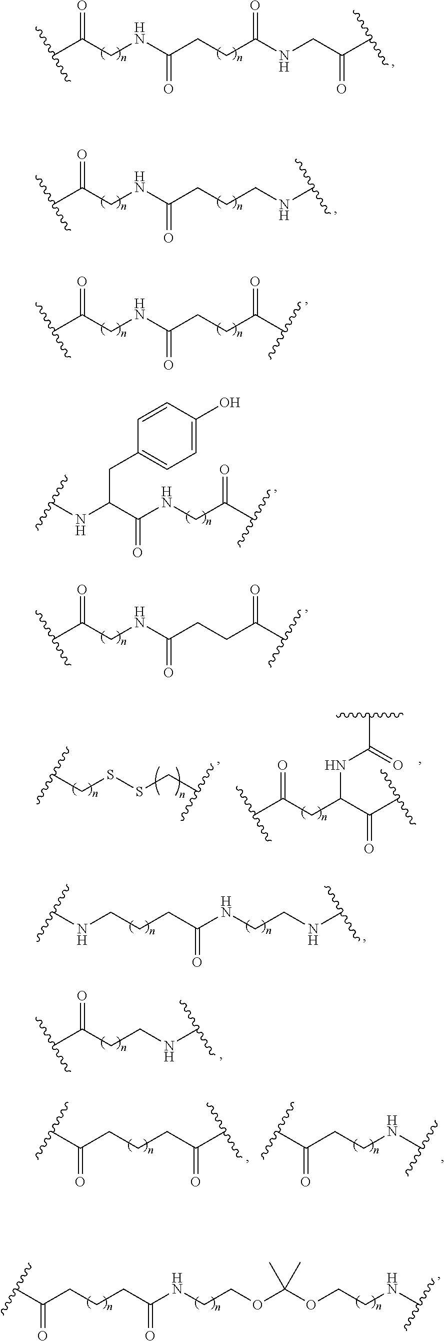

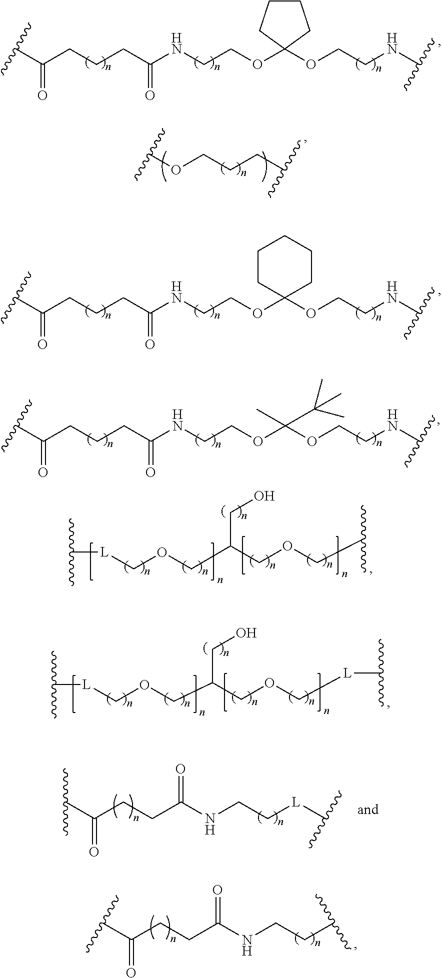

wherein each L is, independently, a phosphorous linking group; and each n is, independently, from 1 to 20.

In certain embodiments, L₁, L₂, L₃, and L₄ (or L₁, L₂, L₃, L₄, L_{1A}, L_{2A}, L_{3A}, and L_{4A}) independently comprise or together comprise the structure selected from among:

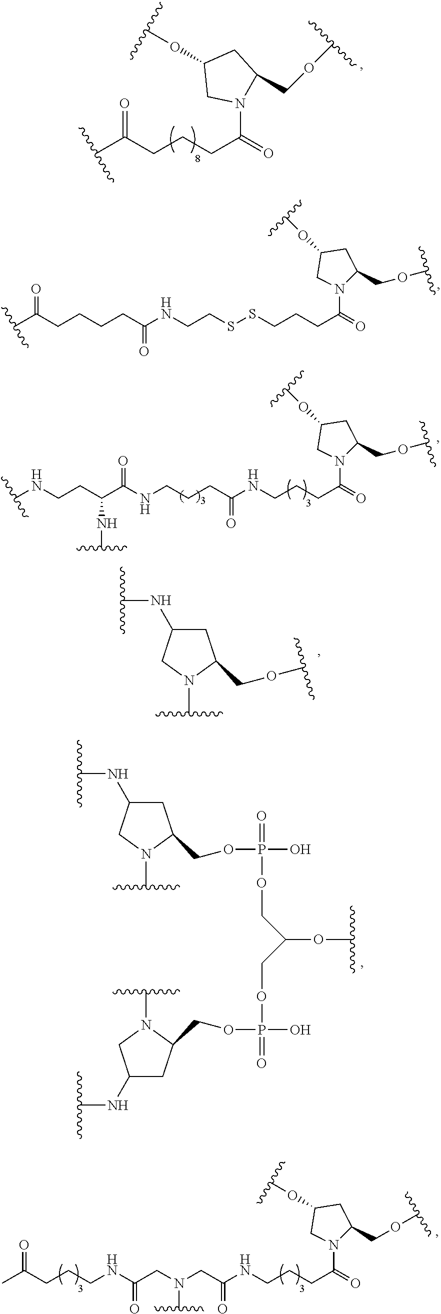

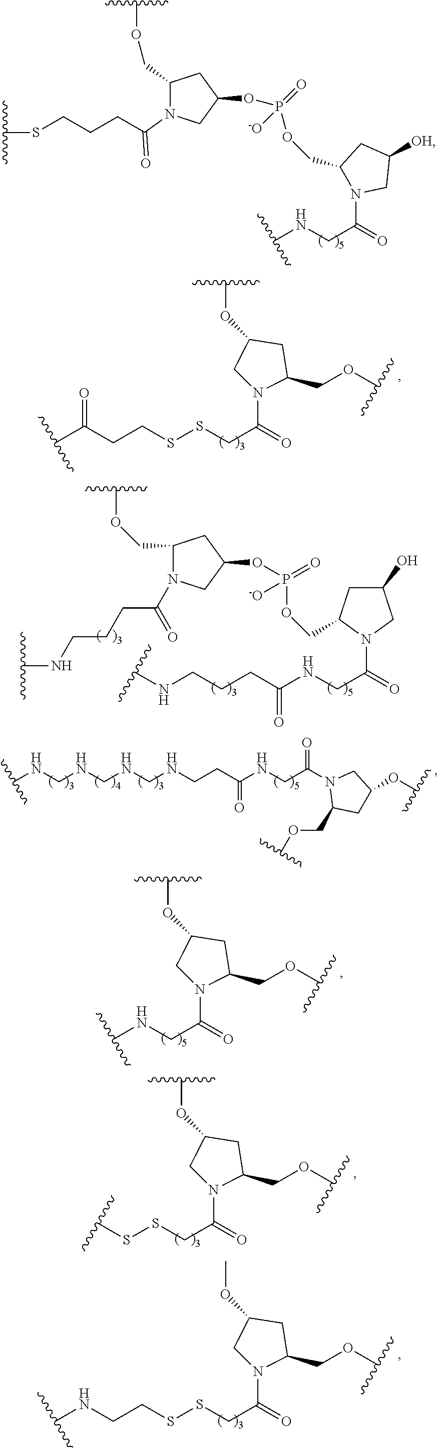

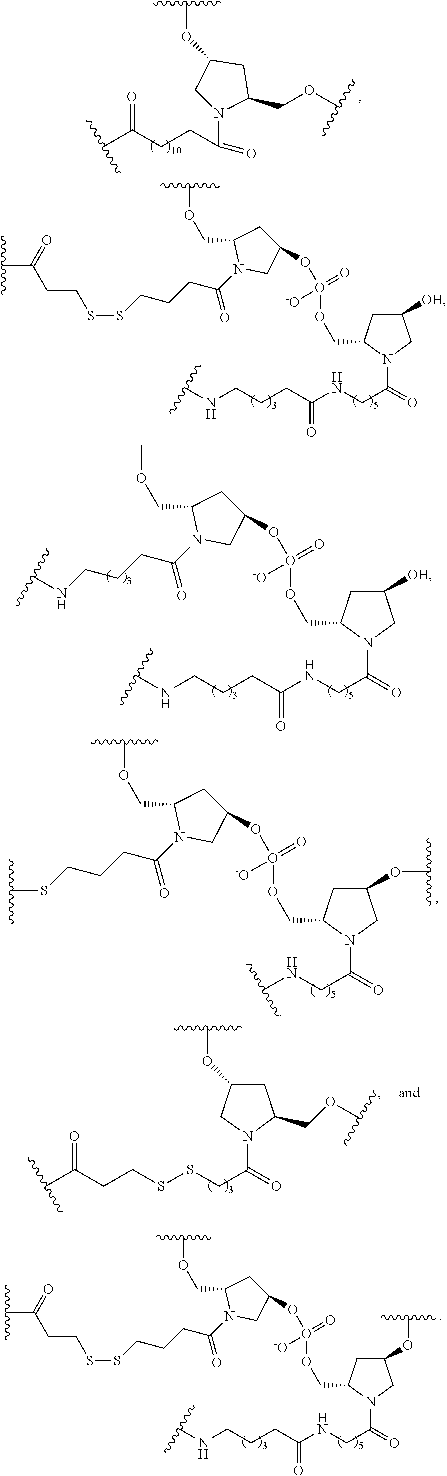

In certain embodiments, L₁, L₂, L₃, and L₄ (or L₁, L₂, L₃, L₄, L_{1A}, L_{2A}, L_{3A}, and L_{4A}) independently comprise or together comprise the structure selected from among:

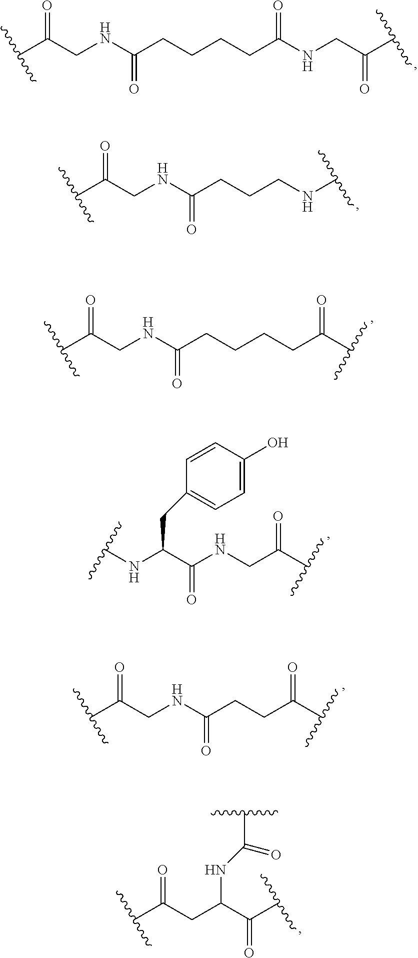

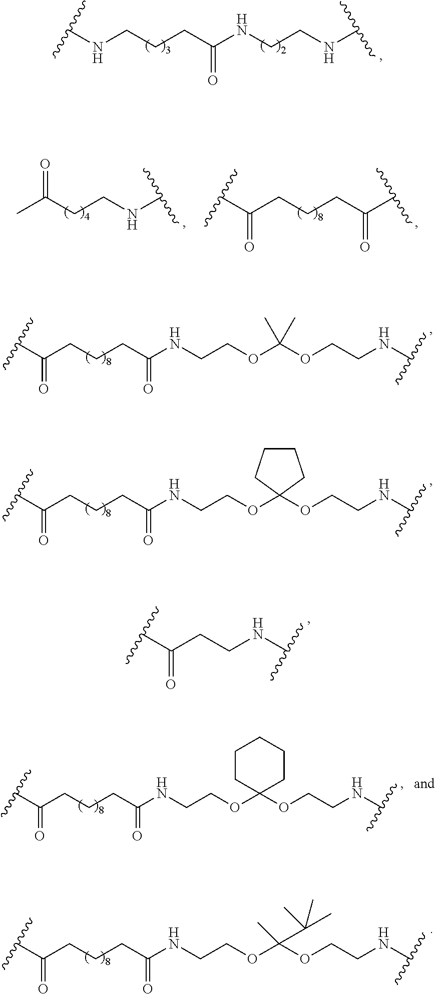

In certain embodiments, L₁, L₂, L₃, and L₄ (or L₁, L₂, L₃, L₄, L₁A, L_{2A}, L_{3A}, and L_{4A}) independently comprise or together comprise the structure selected from among:

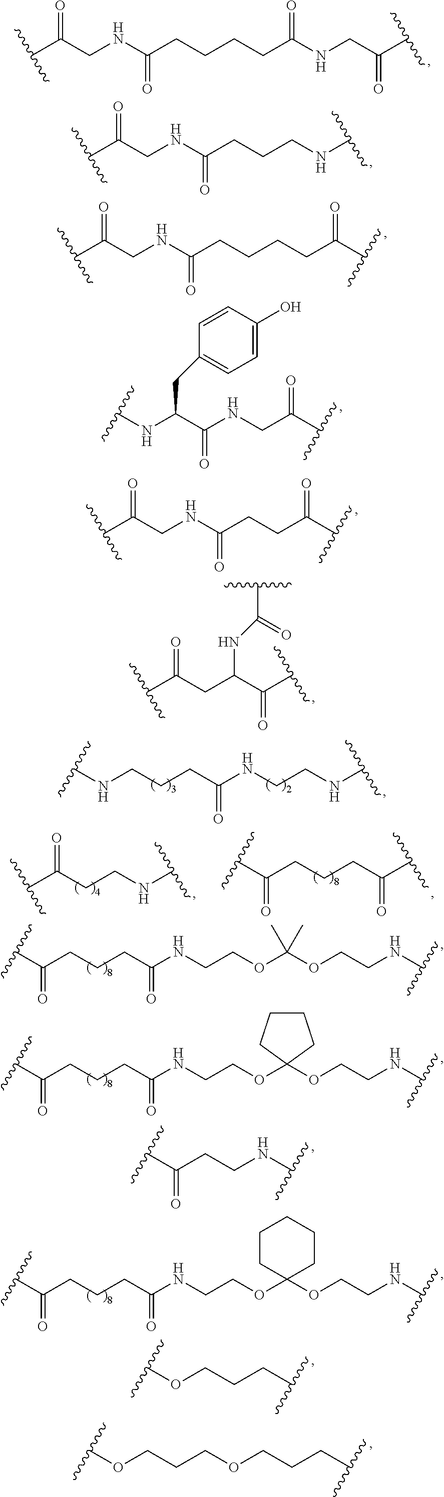

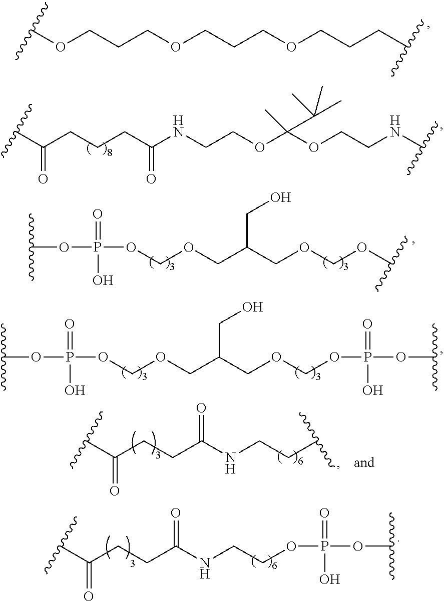

In certain embodiments, L₁, L₂, L₃, and L₄ independently comprise or together comprise the structure selected from among:

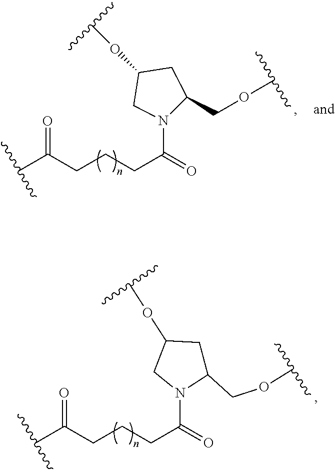

wherein n is from 1 to 20.

In certain embodiments, L₁, L₂, L₃, and L₄ (or L₁, L₂, L₃, L₄, L_{1A}, L_{2A}, L_{3A}, and L_{4A}) independently comprise or together comprise the structure selected from among:

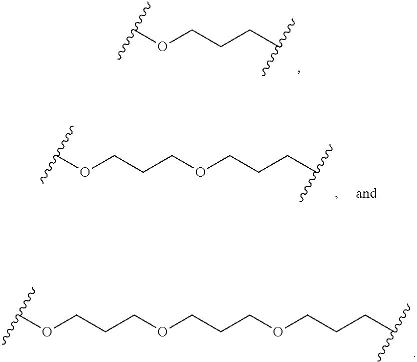 [SMILES: *CCCO* ; *CCCOCCCO* ; *CCCOCCCOCCCO*]

In certain embodiments, L₁, L₂, L₃, and L₄ (or L₁, L₂, L₃, L₄, L_{1A}, L_{2A}, L_{3A}, and L_{4A}) independently comprise or together comprise the structure selected from among:

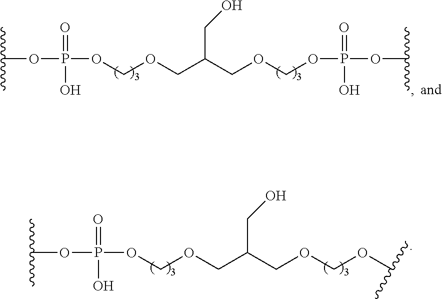

In certain embodiments, L₁, L₂, L₃, and L₄ (or L₁, L₂, L₃, L₄, L_{1A}, L_{2A}, L_{3A}, and L_{4A}) independently comprise or together comprise the structure selected from among:

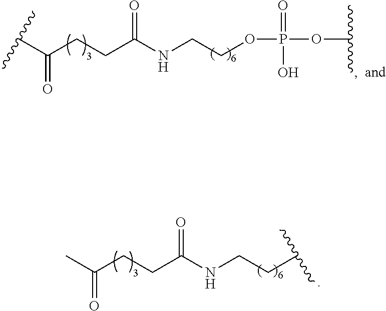

In certain embodiments, L₁, L₂, L₃, and L₄ (or L₁, L₂, L₃, L₄, L_{1A}, L_{2A}, L_{3A}, and L_{4A}) independently comprise or together have the structure:

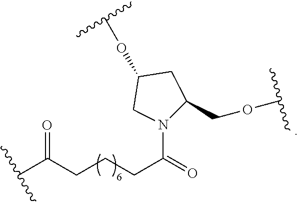

In certain embodiments, L₁, L₂, L₃, and L₄ (or L₁, L₂, L₃, L₄, L_{1A}, L_{2A}, L_{3A}, and L_{4A}) independently comprise or together have the structure:

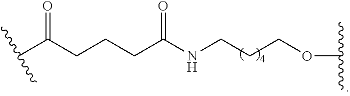

In certain embodiments, L₁, L₂, L₃, and L₄ (or L₁, L₂, L₃, L₄, L_{1A}, L_{2A}, L_{3A}, and L_{4A}) independently comprise or together comprise the structure selected from among:

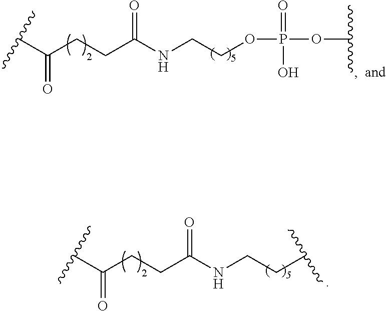

In certain embodiments, L₁, L₂, L₃, and L₄ (or L₁, L₂, L₃, L₄, L_{1A}, L_{2A}, L_{3A}, and L_{4A}) independently comprise or together comprise the structure selected from among:

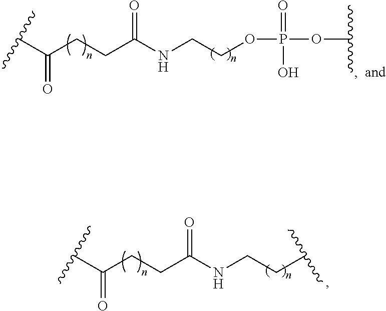

wherein each n is independently 0, 1, 2, 3, 4, 5, 6, or 7.

In some embodiments, any of L₁, L₂, L₃, and L₄ (or L₁, L₂, L₃, L₄, L_{1A}, L_{2A}, L_{3A}, and L_{4A}) may independently be a linker (e.g., an optionally substituted alkyl linker, an optionally substituted polyethylene glycol (PEG) linker, an optionally substituted heteroalkyl linker, or an optionally substituted heteroaryl linker). In some embodiments, any of L₁, L₂, L₃, and L₄ (or L₁, L₂, L₃, L₄, L_{1A}, L_{2A}, L_{3A}, and L_{4A}) may independently be a bond (e.g., a carbon-carbon bond, a phosphodiester bond, or a phosphorothioate bond). In some embodiments, any of L₁, L₂, L₃, and L₄ (or L₁, L₂, L₃, L₄, L_{1A}, L_{2A}, L_{3A}, and L_{4A}) may independently be absent.

In some embodiments, L₁ is a bond.

In some embodiments, L₂ is an optionally substituted PEG linker. In some embodiments, the PEG linker is two, three, four, five, six, seven, eight, nine, or ten PEG units in length. In certain embodiments, L₂ comprises the structure

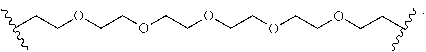 [SMILES: *CCOCCOCCOCCOCCOCC*]

In some embodiments, L₃ is an optionally substituted heteroaryl linker. In some embodiments, L₃ is an optionally substituted partially unsaturated heteroaryl linker. In certain

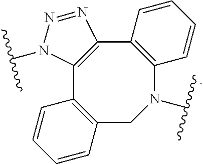 [SMILES: *N1Cc2ccccc2-c2c(nnn2*)-c2ccccc21]

embodiments, L₃ comprises the structure

In some embodiments, L₄ is an optionally substituted heteroalkyl linker. In some embodiments, the heteroalkyl linker is substituted with one or more ═O substituents. In some embodiments, the heteroalkyl linker comprises two substituents joined together to form an optionally substituted carbocyclyl ring. In certain embodiments, L₄ comprises the structure

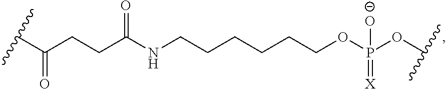

wherein X is O or S. In certain embodiments, L₄ comprises the structure

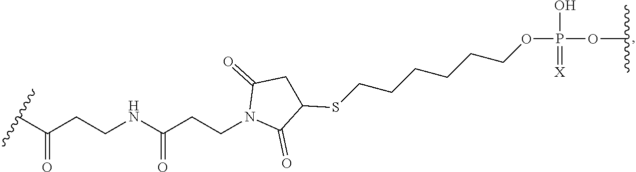

wherein X is

In some embodiments, L₁, L₂, L₃, and L₄ and/or L_{1A}, L_{2A}, L_{3A}, and L_{4A} together comprise the structure

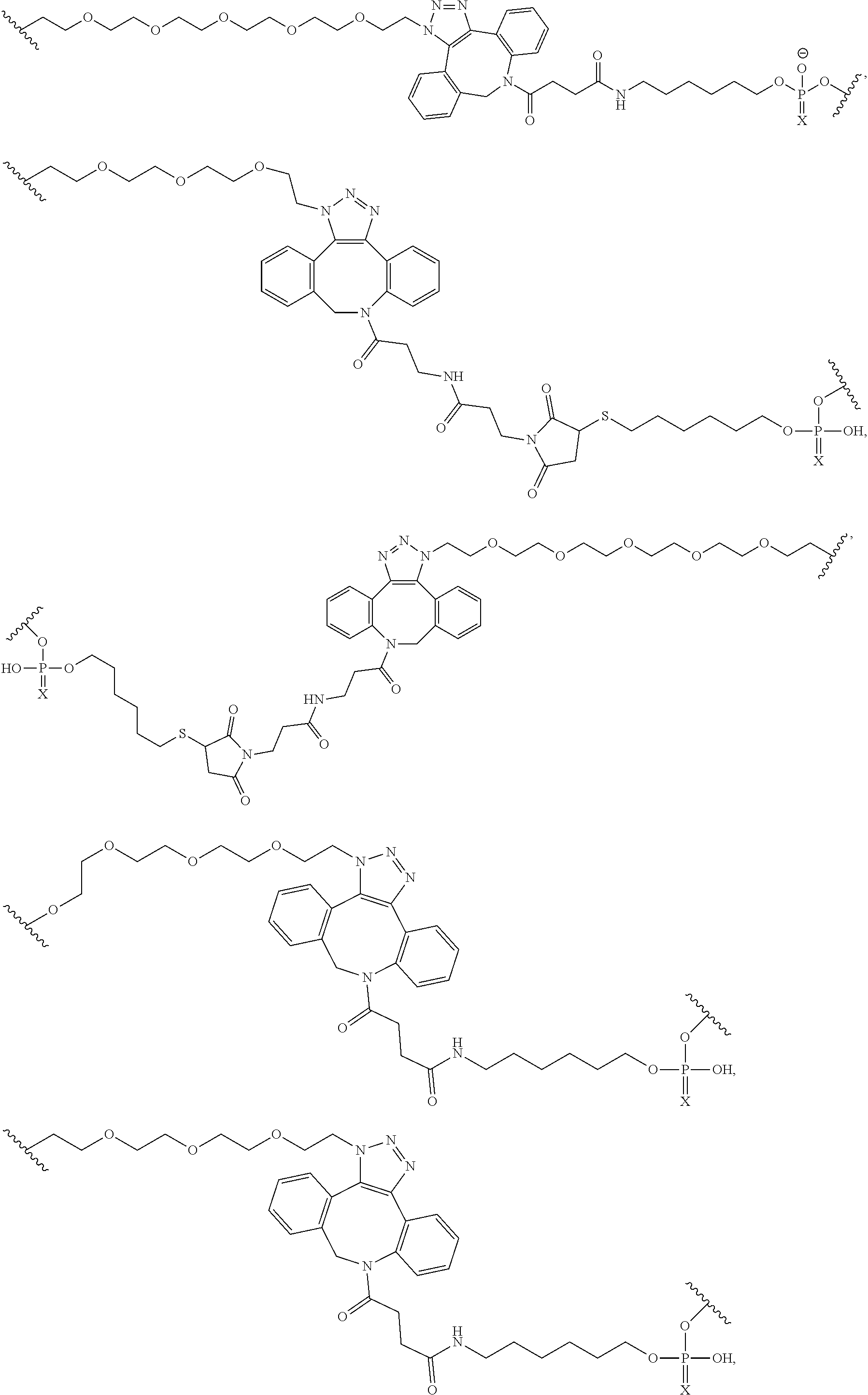

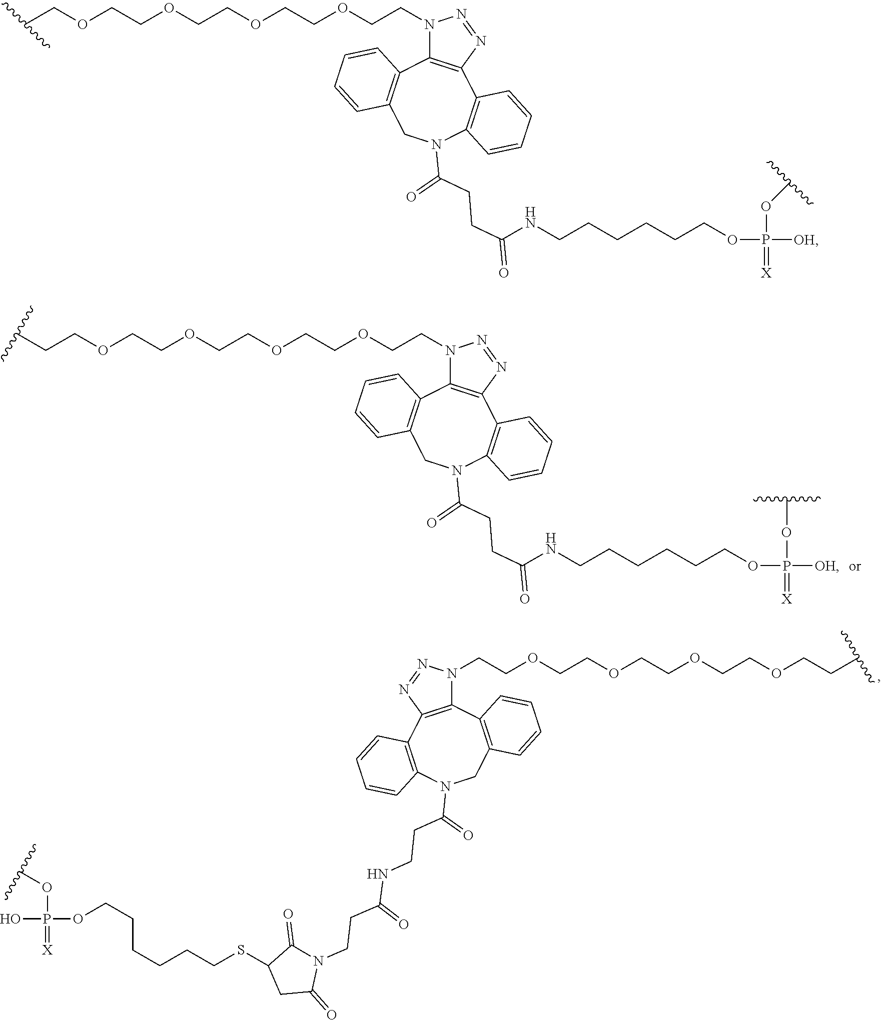

wherein X is O or S.,

### Methods of Making Compounds

In some aspects, the disclosure relates to methods of making the compounds and compositions comprising α₄β₁in integrin receptor ligands as disclosed herein.

Compounds of the present disclosure can be made by means known in the art of organic synthesis. Methods for optimizing reaction conditions, and minimizing competing by-products, if necessary, are known in the art. Reaction optimization and scale-up may advantageously utilize high-speed parallel synthesis equipment and computer-controlled microreactors (e.g., Design and Optimization in Organic Synthesis, 2^{nd} Edition, Carlson R, Ed, 2005; Elsevier Science Ltd.; Jahnisch, K et al., Angew. Chem. Int. Ed. Engl. 2004 43: 406; and references therein). Additional reaction schemes and protocols may be determined by the skilled artisan by use of commercially available structure-searchable database software, for instance, SciFinder@(CAS division of the American Chemical Society) and Reaxys® (Elsevier), or by appropriate keyword searching using an internet search engine such as Google® or keyword databases such as the U.S. Patent and Trademark Office text database.

As can be appreciated by the skilled artisan, methods of synthesizing the compounds of the formulae herein will be evident to those of ordinary skill in the art, including in the schemes and examples herein. Additionally, the various synthetic steps may be performed in an alternate sequence or order to give the desired compounds. In addition, the solvents, temperatures, reaction durations, etc. delineated herein are for purposes of illustration only and one of ordinary skill in the art will recognize that variation of the reaction conditions can produce the desired compounds of the present disclosure.

The compounds herein may also contain linkages (e.g., carbon-carbon bonds) wherein bond rotation is restricted about that particular linkage, e.g., restriction resulting from the presence of a ring or double bond. Accordingly, all cis/trans and E/Z isomers are expressly included in the present disclosure. The compounds herein may also be represented in multiple tautomeric forms; in such instances, the present disclosure expressly includes all tautomeric forms of the compounds described herein, even though only a single tautomeric form may be represented. All such isomeric forms of such compounds herein are expressly included in the present disclosure. All crystal forms and polymorphs of the compounds described herein are expressly included in the present disclosure. Also embodied are extracts and fractions comprising compounds of the present disclosure. The term “isomers” is intended to include diastereoisomers, enantiomers, regioisomers, structural isomers, rotational isomers, tautomers, and the like. For compounds which contain one or more stereogenic centers, e.g., chiral compounds, the methods of the present disclosure may be carried out with an enantiomerically enriched compound, a racemate, or a mixture of diastereomers. All isomers of compounds delineated herein are expressly included in the present disclosure.

Preferred enantiomerically enriched compounds have an enantiomeric excess of 50% or more. More preferably, the compound has an enantiomeric excess of 60%, 70%, 80%, 90%, 95%, 98%, 99%, or more. In preferred embodiments, only one enantiomer or diastereomer of a chiral compound of the present disclosure is administered to cells or a subject.

### Methods of Treatment

In one aspect, provided are methods of treating a subject suffering from or susceptible to a disorder or disease, comprising administering to the subject an effective amount of a compound or pharmaceutical composition described herein.

In other aspects, provided are methods of treating a subject suffering from or susceptible to a disorder or disease, wherein the subject has been identified as in need of modulation of the function of a protein, comprising administering to said subject in need thereof, an effective amount of a compound or pharmaceutical composition described herein, such that said subject is treated for said disorder.

In one aspect, provided are methods of delivering a therapeutic oligonucleotide to the brain of a subject, comprising contacting the subject with a compound or pharmaceutical composition described herein, in an amount and under conditions sufficient to target the brain. In some embodiments, the therapeutic oligonucleotide is delivered to one or more brain regions selected from the group consisting of the striatum, the cerebellum, the brain stem, the hippocampus, the frontal cortex, and the spinal cord.

In certain embodiments, provided are methods of treating a disease, disorder or symptom thereof, wherein the disease is a central nervous system (CNS) disease, disorder, or symptom thereof. In some embodiments, the disease is a neurodegenerative disease, disorder, or symptom thereof. In some embodiments, the disease is Alzheimer's disease, or a symptom thereof.

Exemplary CNS disorders include, but are not limited to, neurotoxicity and/or neurotrauma, stroke, multiple sclerosis, spinal cord injury, epilepsy, a mental disorder, a sleep condition, a movement disorder, nausea and/or emesis, amyotrophic lateral sclerosis, Alzheimer's disease, and drug addiction.

In certain embodiments, the CNS disorder is neurotoxicity and/or neurotrauma, e.g., for example, as a result of acute neuronal injury (e.g., traumatic brain injury (TBI), stroke, epilepsy) or a chronic neurodegenerative disorder (e.g., multiple sclerosis, Parkinson's disease, Huntington's disease, amyotrophic lateral sclerosis, Alzheimer's disease). In certain embodiments, the compounds of the present disclosure provide a neuroprotective effect, e.g., against an acute neuronal injury or a chronic neurodegenerative disorder.

In certain embodiments, the CNS disorder is stroke (e.g., ischemic stroke).

In certain embodiments, the CNS disorder is multiple sclerosis.

In certain embodiments, the CNS disorder is spinal cord injury.

In certain embodiments, the CNS disorder is epilepsy.

In certain embodiments, the CNS disorder is a mental disorder, e.g., for example, depression, anxiety or anxiety-related conditions, a learning disability, a somatic system disorder, schizophrenia, or schizoaffective disorder.

In certain embodiments, the CNS disorder is depression. “Depression” includes, but is not limited to, depressive disorders or conditions, such as, for example, major depressive disorders (e.g., unipolar depression), treatment-resistant depression, dysthymic disorders (e.g., chronic, mild depression), bipolar disorders (e.g., manic-depression), seasonal affective disorder, and/or depression associated with substance abuse or substance abuse disorder (e.g., withdrawal). The depression can be clinical or subclinical depression. The depression can be associated with or premenstrual syndrome and/or premenstrual dysphoric disorder.

In certain embodiments, the CNS disorder is anxiety. “Anxiety” includes, but is not limited to, anxiety and anxiety-related conditions, such as, for example, clinical anxiety, panic disorder, agoraphobia, generalized anxiety disorder (GAD), specific phobia, social phobia, obsessive-compulsive disorder, acute stress disorder, post-traumatic stress disorder, adjustment disorders with anxious features, anxiety disorder associated with depression, anxiety disorder due to general medical conditions, and substance-induced anxiety disorders, anxiety associated with substance abuse or substance use disorder (e.g., withdrawal, dependence, reinstatement) and anxiety associated with nausea and/or emesis. This treatment may also be to induce or promote sleep in a subject (e.g., for example, a subject with anxiety).

In certain embodiments, the CNS disorder is a learning disorder (e.g., attention deficit disorder (ADD)).

In certain embodiments, the CNS disorder is schizophrenia or schizoaffective disorder.

In certain embodiments, the CNS disorder is a sleep condition. “Sleep conditions” include, but are not limited to, insomnia, narcolepsy, sleep apnea, restless legs syndrome (RLS), delayed sleep phase syndrome (DSPS), periodic limb movement disorder (PLMD), hypopnea syndrome, rapid eye movement behavior disorder (RBD), shift work sleep condition (SWSD), and sleep problems (e.g., parasomnias) such as nightmares, night terrors, sleep talking, head banging, snoring, and clenched jaw and/or grinding of teeth (bruxism).

In certain embodiments, the CNS disorder is a movement disorder, e.g., basal ganglia disorders, such as, for example, Parkinson's disease, levodopa-induced dyskinesia, Huntington's disease, Gilles de Ia Tourette's syndrome, tardive dyskinesia, and dystonia.

In certain embodiments, the CNS disorder is Alzheimer's disease.

In certain embodiments, the CNS disorder is amyotrophic lateral sclerosis (ALS).

In certain embodiments, the CNS disorder is nausea and/or emesis.

In certain embodiments, the CNS disorder is drug addiction (e.g., for instance, addiction to opiates, nicotine, cocaine, psychostimulants, or alcohol).

The term “neurological disease” (including, e.g., “neurodegenerative diseases”) refers to any disease of the nervous system, including diseases that involve the central nervous system (brain, brainstem and cerebellum), the peripheral nervous system (including cranial nerves), and the autonomic nervous system (parts of which are located in both central and peripheral nervous system). Neurodegenerative diseases refer to a type of neurological disease marked by the loss of nerve cells, including, but not limited to, Alzheimer's disease, Parkinson's disease, amyotrophic lateral sclerosis, tauopathies (including frontotemporal dementia), and Huntington's disease. Examples of neurological diseases include, but are not limited to, headache, stupor and coma, dementia, seizure, sleep disorders, trauma, infections, neoplasms, neuro-ophthalmology, movement disorders, demyelinating diseases, spinal cord disorders, and disorders of peripheral nerves, muscle, and neuromuscular junctions. Substance abuse or substance use disorder (SUD) and mental illness, including, but not limited to, bipolar disorder, and schizophrenia, and schizoaffective disorder, are also included in the definition of neurological diseases. Further examples of neurological diseases include acquired epileptiform aphasia; acute disseminated encephalomyelitis; adrenoleukodystrophy; agenesis of the corpus callosum; agnosia; Aicardi syndrome; Alexander disease; Alpers' disease; alternating hemiplegia; Alzheimer's disease; amyotrophic lateral sclerosis; anencephaly; Angelman syndrome; angiomatosis; anoxia; aphasia; apraxia; arachnoid cysts; arachnoiditis; Arnold-Chiari malformation; arteriovenous malformation; Asperger syndrome; ataxia telangiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; brain injury; brain tumors (including glioblastoma multiforme); spinal tumor; Brown-Sequard syndrome; Canavan disease; carpal tunnel syndrome (CTS); causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy (CIDP); chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease (CIBD); cytomegalovirus infection; dancing eyes-dancing feet syndrome; Dandy-Walker syndrome; Dawson disease; De Morsier's syndrome; Dejerine-Klumpke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; frontotemporal dementia and other “tauopathies”; Gaucher's disease; Gerstmann's syndrome; giant cell arteritis; giant cell inclusion disease; globoid cell leukodystrophy; Guillain-Barre syndrome; HTLV-1 associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (see also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile; phytanic acid storage disease; Infantile Refsum disease; infantile spasms; inflammatory myopathy; intracranial cyst; intracranial hypertension; Joubert syndrome; Kearns-Sayre syndrome; Kennedy disease; Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; Lewy body dementia; lissencephaly; locked-in syndrome; Lou Gehrig's disease (also known as motor neuron disease or amyotrophic lateral sclerosis); lumbar disc disease; lyme disease-neurological sequelae; Machado-Joseph disease; macrencephaly; megalencephaly; Melkersson-Rosenthal syndrome; Meniere's disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neurone disease; moyamoya disease; mucopolysaccharidoses; multi-infarct dementia; multifocal motor neuropathy; multiple sclerosis and other demyelinating disorders; multiple system atrophy with postural hypotension; muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenita; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; Post-Polio syndrome; postherpetic neuralgia (PHN); postinfectious encephalomyelitis; postural hypotension; Prader-Willi syndrome; primary lateral sclerosis; prion diseases; progressive; hemifacial atrophy; progressive multifocal leukoencephalopathy; progressive sclerosing poliodystrophy; progressive supranuclear palsy; pseudotumor cerebri; Ramsay-Hunt syndrome (Type I and Type II); Rasmussen's Encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus Dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; stiff-person syndrome; stroke; Sturge-Weber syndrome; subacute sclerosing panencephalitis; subarachnoid hemorrhage; subcortical arteriosclerotic encephalopathy; sydenham chorea; syncope; syringomyelia; tardive dyskinesia; Tay-Sachs disease; temporal arteritis; tethered spinal cord syndrome; Thomsen disease; thoracic outlet syndrome; tic douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau Disease (VHL); Wallenberg's syndrome; Werdnig-Hoffman disease; West syndrome; whiplash; Williams syndrome; Wilson's disease; and Zellweger syndrome.

In certain embodiments, the subject is a mammal, preferably a primate or a human.

In another embodiment, provided are methods as described above, wherein the effective amount of the compounds provided herein is as described above.

In another embodiment, provided are methods as described above, wherein the compounds provided herein is administered intrathecally, intravenously, intramuscularly, subcutaneously, intracerebroventricularly, orally, or topically. In certain embodiments, the compound is administered intrathecally.

In other embodiments, provided are methods as described above, wherein the compound of any of the formulae provided herein is administered alone or in combination with one or more other therapeutics. In a further embodiment, the additional therapeutic agent is a central nervous system (CNS) disease agent.

Another object of the present disclosure is the use of a compound as described herein in the manufacture of a medicament for use in the treatment of a disorder or disease. Another object of the present disclosure is the use of a compound as described herein for use in the treatment of a disorder or disease.

### Pharmaceutical Compositions

In one aspect, provided are pharmaceutical compositions comprising any of the compounds described herein and a pharmaceutically acceptable carrier or pharmaceutically acceptable excipient.

A compound or composition, as described herein, can be administered in combination with one or more additional therapeutic agents (e.g., therapeutically and/or prophylactically active agents). The compounds or compositions can be administered in combination with additional therapeutic agents that improve their activity (e.g., activity (e.g., potency and/or efficacy) in treating a disease in a subject in need thereof, in preventing a disease in a subject in need thereof, and/or in reducing the risk to develop a disease in a subject in need thereof), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound described herein and an additional therapeutic agent exhibits a synergistic effect that is absent in a pharmaceutical composition including one of the compounds described herein or the additional therapeutic agent, but not both.

The compound or composition can be administered concurrently with, prior to, or subsequent to one or more additional therapeutic agents, which may be useful as, e.g., combination therapies. Therapeutic agents include therapeutically active agents. Therapeutic agents also include prophylactically active agents. Therapeutic agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional therapeutic agent is a therapeutic agent useful for treating and/or preventing a disease (e.g., CNS disorder). Each additional therapeutic agent may be administered at a dose and/or on a time schedule determined for that therapeutic agent. The additional therapeutic agents may also be administered together with each other and/or with the compound or composition described herein in a single dose or administered separately in different doses. The particular combination to employ in a regimen will take into account compatibility of the compound described herein with the additional therapeutic agent(s) and/or the desired therapeutic and/or prophylactic effect to be achieved. In general, it is expected that the additional therapeutic agent(s) in combination be utilized at levels that do not exceed the levels at which they are utilized individually. In some embodiments, the levels utilized in combination will be lower than those utilized individually.

In one aspect, provided are kits comprising an effective amount of a compound provided herein, in unit dosage form, together with instructions for administering the compound to a subject suffering from or susceptible to a disease or disorder.

The term “pharmaceutically acceptable salts” or “pharmaceutically acceptable carrier” is meant to include salts of the active compounds which are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydroiodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present disclosure contain both basic and acidic functionalities that allow the compounds to be converted into either base or acid addition salts. Other pharmaceutically acceptable carriers known to those of skill in the art are suitable for the present disclosure.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present disclosure.

In addition to salt forms, the present disclosure provides compounds which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

Certain compounds of the present disclosure can exist in unsolvated forms as well as solvated forms, including hydrated forms. In general, the solvated forms are equivalent to unsolvated forms and are intended to be encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

The present disclosure also provides a pharmaceutical composition, comprising an effective amount of a compound described herein and a pharmaceutically acceptable excipient. In an embodiment, a compound of any of the formulae provided herein is administered to a subject using a pharmaceutically acceptable formulation, e.g., a pharmaceutically-acceptable formulation that provides sustained delivery of the compound to a subject for at least 12 hours, 24 hours, 36 hours, 48 hours, one week, two weeks, three weeks, or four weeks after the pharmaceutically-acceptable formulation is administered to the subject.

Actual dosage levels and time course of administration of the active ingredients in the pharmaceutical compositions of the disclosure may be varied so as to obtain an amount of the active ingredient which is effective to achieve the desired therapeutic response for a particular subject, composition, and mode of administration, while being acceptably tolerant to the subject.

In use, at least one compound according to the present disclosure is administered in a pharmaceutically effective amount to a subject in need thereof in a pharmaceutical carrier by intravenous, intrathecal, intramuscular, subcutaneous, or intracerebroventricular injection or by oral administration or topical application. In accordance with the present disclosure, a compound of the disclosure may be administered alone or in conjunction with a second, different therapeutic. By “in conjunction with” is meant together, substantially simultaneously, or sequentially. In one embodiment, a compound of the disclosure is administered acutely. The compound of the disclosure may therefore be administered for a short course of treatment, such as for about 1 day to about 1 week. In another embodiment, the compound of the disclosure may be administered over a longer period of time to ameliorate chronic disorders, such as, for example, for about one week to several months depending upon the condition to be treated.

By “pharmaceutically effective amount,” as used herein, is meant an amount of a compound of the disclosure, high enough to significantly positively modify the condition to be treated but low enough to avoid serious side effects (at a reasonable benefit/risk ratio), within the scope of sound medical judgment. A pharmaceutically effective amount of a compound of the disclosure will vary with the particular goal to be achieved, the age and physical condition of the patient being treated, the severity of the underlying disease, the duration of treatment, the nature of concurrent therapy and the specific compound employed. For example, a therapeutically effective amount of a compound of the disclosure administered to a child or a neonate will be reduced proportionately in accordance with sound medical judgment. The effective amount of a compound of the disclosure will thus be the minimum amount which will provide the desired effect.

A decided practical advantage of the present disclosure is that the compound may be administered in a convenient manner such as by intrathecal, intravenous, intramuscular, subcutaneous, oral, or intra-cerebroventricular injection routes or by topical application, such as in creams or gels. Depending on the route of administration, the active ingredients which comprise a compound of the disclosure may be required to be coated in a material to protect the compound from the action of enzymes, acids and other natural conditions which may inactivate the compound. In order to administer a compound of the disclosure by a mode other than parenteral administration, the compound can be coated by, or administered with, a material to prevent inactivation.

The compound may be administered parenterally or intraperitoneally. Dispersions can also be prepared, for example, in glycerol, liquid polyethylene glycols, and mixtures thereof, and in oils.

Some examples of substances which can serve as pharmaceutical excipients, or pharmaceutical carriers (which terms are used interchangeably herein), are sugars, such as lactose, glucose and sucrose; starches such as corn starch and potato starch; cellulose and its derivatives such as sodium carboxymethycellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil, corn oil, and oil of theobroma; polyols such as propylene glycol, glycerine, sorbitol, mannitol, and polyethylene glycol; agar; alginic acids; pyrogen-free water; isotonic saline; and phosphate buffer solution; skim milk powder; as well as other non-toxic compatible substances used in pharmaceutical formulations such as Vitamin C, estrogen and echinacea, for example. Wetting agents and lubricants such as sodium lauryl sulfate, as well as coloring agents, flavoring agents, lubricants, excipients, tableting agents, stabilizers, antioxidants, and preservatives, can also be present. Solubilizing agents, including for example, cremaphore, and beta-cyclodextrins, can also be used in the pharmaceutical compositions herein.

Pharmaceutical compositions comprising the active compounds of the present disclosure (or derivatives or prodrugs thereof) can be manufactured by means of conventional mixing, dissolving, granulating, dragee-making levigating, emulsifying, encapsulating, entrapping, or lyophilization processes. The compositions can be formulated in conventional manner using one or more physiologically acceptable carriers, diluents, excipients, or auxiliaries, which facilitate processing of the active compounds into preparations that can be used pharmaceutically. The compositions herein can be made by combining (e.g., contacting, mixing, dissolving, granulating, dragee-making, levigating, emulsifying, encapsulating, entrapping, or lyophilizing) a compound delineated herein with one or more suitable carriers, diluents, excipients, or auxiliaries, including those described herein (e.g., for pharmaceutical, agricultural, or veterinary use).

Pharmaceutical compositions of the present disclosure can take a form suitable for virtually any mode of administration, including, for example, intrathecal, topical, ocular, oral, buccal, systemic, nasal, injection, transdermal, rectal, vaginal, and the like, or a form suitable for administration by inhalation or insufflation.

Systemic formulations include those designed for administration by injection, e.g., subcutaneous, intravenous, intramuscular, intrathecal, or intraperitoneal injection, as well as those designed for transdermal, transmucosal, oral, or pulmonary administration.

Useful injectable preparations include sterile suspensions, solutions, or emulsions of the active compound(s) in aqueous or oily vehicles. The compositions also can contain formulating agents, such as suspending, stabilizing and/or dispersing agent. The formulations for injection can be presented in unit dosage form (e.g., in ampules or in multidose containers) and can contain added preservatives.

Alternatively, the injectable formulation can be provided in powder form for reconstitution with a suitable vehicle, including but not limited to, sterile pyrogen free water, buffer, dextrose solution, and the like, before use. To this end, the active compound(s) can be dried by any art-known technique, such as lyophilization, and reconstituted prior to use.

For prolonged delivery, the active compound(s), or prodrug(s) can be formulated as a depot preparation for administration by implantation or intramuscular injection. The active ingredient can be formulated with suitable polymeric or hydrophobic materials (e.g., as an emulsion in an acceptable oil) or ion exchange resins, or as sparingly soluble derivatives, e.g., as a sparingly soluble salt.

Alternatively, other pharmaceutical delivery systems can be employed. Liposomes and emulsions are well-known examples of delivery vehicles that can be used to deliver active compound(s), oligonucleotide(s), or prodrug(s). Certain organic solvents such as dimethylsulfoxide (DMSO) also can be employed.

The pharmaceutical compositions can, if desired, be presented in a pack or dispenser device that can contain one or more unit dosage forms containing the active compound(s). The pack can, for example, comprise metal or plastic foil, such as a blister pack. The pack or dispenser device can be accompanied by instructions for administration.

The active compound(s), or prodrug(s) of the present disclosure, or compositions thereof, will generally be used in an amount effective to achieve the intended result, for example in an amount effective to treat or prevent the particular disease being treated. The compound(s) and oligonucleotide(s) can be administered therapeutically to achieve therapeutic benefit or prophylactically to achieve prophylactic benefit. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient can still be afflicted with the underlying disorder. Therapeutic benefit also includes halting or slowing the progression of the disease, regardless of whether improvement is realized.

For prophylactic administration, the compound can be administered to a patient at risk of developing one of the previously described diseases. A patient at risk of developing a disease can be a patient having characteristics placing the patient in a designated group of at-risk patients, as defined by an appropriate medical professional or group. A patient at risk may also be a patient that is commonly or routinely in a setting where development of the underlying disease could occur. In other words, an at-risk patient is one who is commonly or routinely exposed to the disease or illness causing conditions or may be acutely exposed for a limited time. Alternatively, prophylactic administration can be applied to avoid the onset of symptoms in a patient diagnosed with the underlying disorder.

The amount of compound administered will depend upon a variety of factors, including, for example, the particular indication being treated, the mode of administration, whether the desired benefit is prophylactic or therapeutic, the severity of the indication being treated, the age and weight of the patient, the bioavailability of the particular active compound, and the like. Determination of an effective dosage is well within the capabilities of those skilled in the art.

Effective dosages can be estimated initially from in vitro assays. For example, an initial dosage for use in animals can be formulated to achieve a circulating blood or serum concentration of active compound that is at or above an IC₅₀ of the particular compound as measured in an in vitro assay, such as an in vitro fungal MIC or MFC, and other in vitro assays. Calculating dosages to achieve such circulating blood or serum concentrations taking into account the bioavailability of the particular compound is well within the capabilities of skilled artisans. For guidance, see “General Principles,” In: Goodman and Gilman's The Pharmaceutical Basis of Therapeutics, Chapter 1, pp. 1-112, 13th ed., McGraw-Hill, and the references cited therein, which are incorporated herein by reference.

Initial dosages also can be estimated from in vivo data, such as animal models. Animal models useful for testing the efficacy of compounds to treat or prevent the various diseases described above are well-known in the art.

Dosage amounts will typically be in the range of from about 0.0001 or 0.001 or 0.01 mg/kg/day to about 100 mg/kg/day, but can be higher or lower, depending upon, among other factors, the activity of the compound, its bioavailability, the mode of administration, and various factors discussed above. Dosage amount and interval can be adjusted individually to provide plasma levels of the compound(s) that are sufficient to maintain therapeutic or prophylactic effect. In cases of local administration or selective uptake, such as local topical administration, the effective local concentration of active compound(s) cannot be related to plasma concentration. Skilled artisans will be able to optimize effective local dosages without undue experimentation.

Preferably, the compound(s) will provide therapeutic or prophylactic benefit and will have acceptable tolerability. Tolerability of the compound(s) and oligonucleotide(s) can be determined using standard pharmaceutical procedures. The dose ratio between non-tolerable and therapeutic (or prophylactic) effect is the therapeutic index. Compounds(s) that exhibit high therapeutic indices are preferred.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

### Additional Embodiments

Certain embodiments include embodiment P1 to embodiment P30 following:

Embodiment P1. A compound comprising the structure of Formula (I), or a salt thereof:

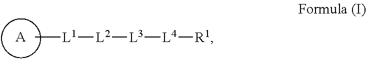

wherein

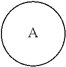

is an α₄β_{1/7} integrin ligand; each of L₁, L₂, L₃, and L₄ is independently a linker, a bond, or absent; and R¹ comprises one or more oligonucleotides, protecting groups, small molecules, proteins, antibodies, or peptides.

Embodiment P2. The compound, or salt thereof, of embodiment 1, wherein the α₄β_{1/7} integrin ligand is an α₄ in integrin agonist.

Embodiment P3. The compound, or salt thereof, of embodiment 1, wherein the α₄β_{1/7} integrin ligand is an α₄ in integrin antagonist.

Embodiment P4. The compound, or salt thereof, of embodiment 1, wherein the α₄β_{1/7} integrin ligand is selected from the group consisting of:

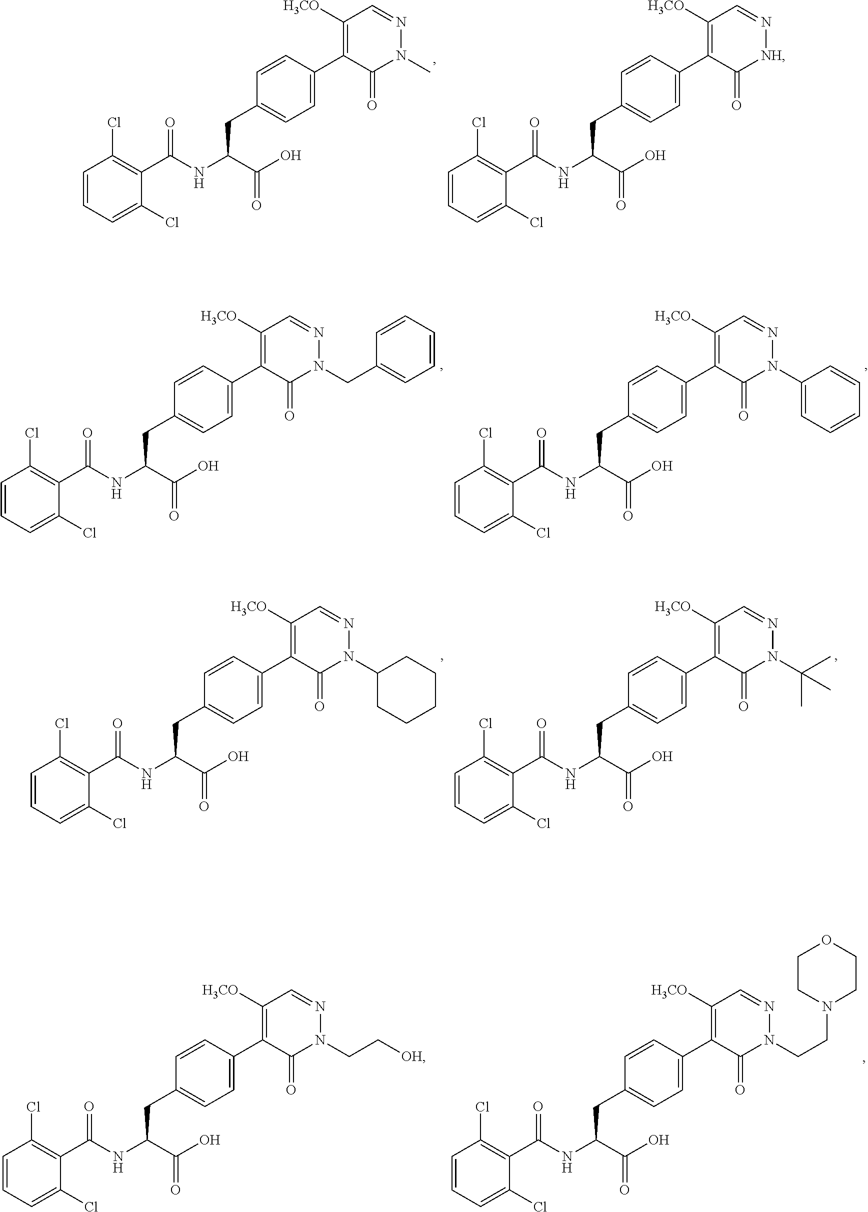

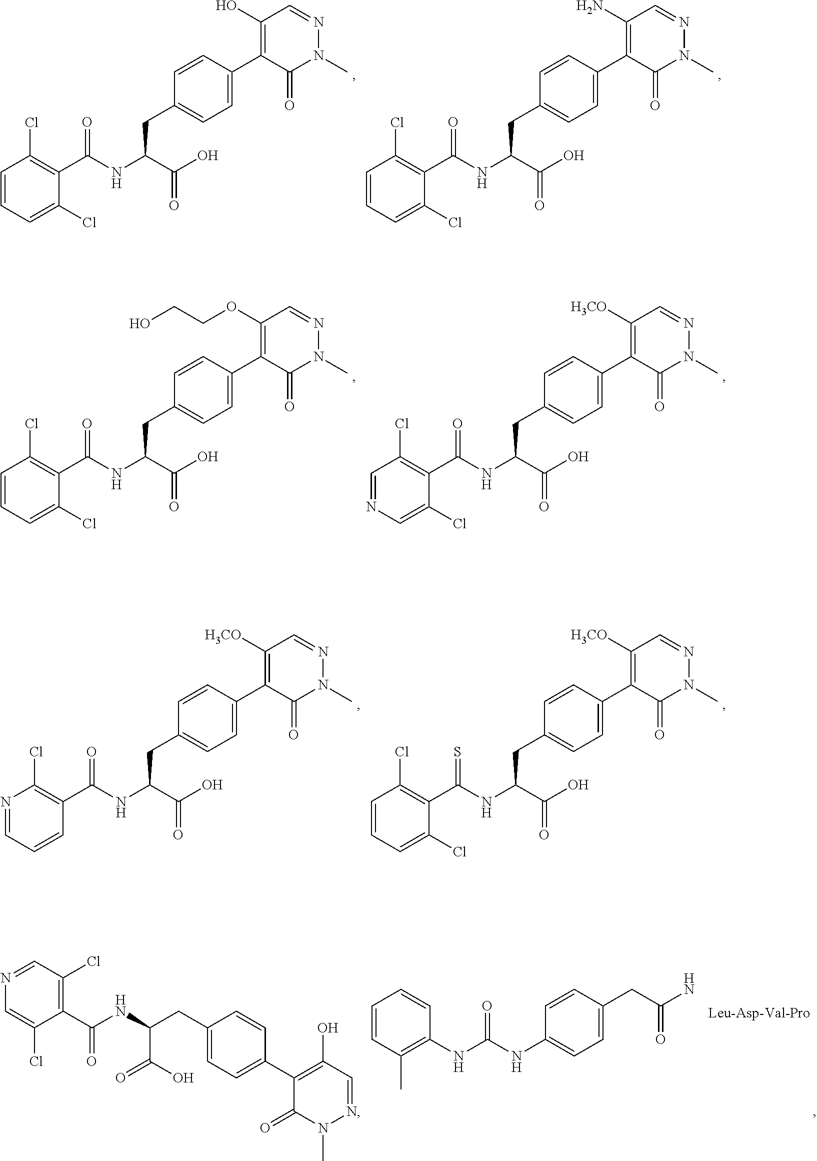

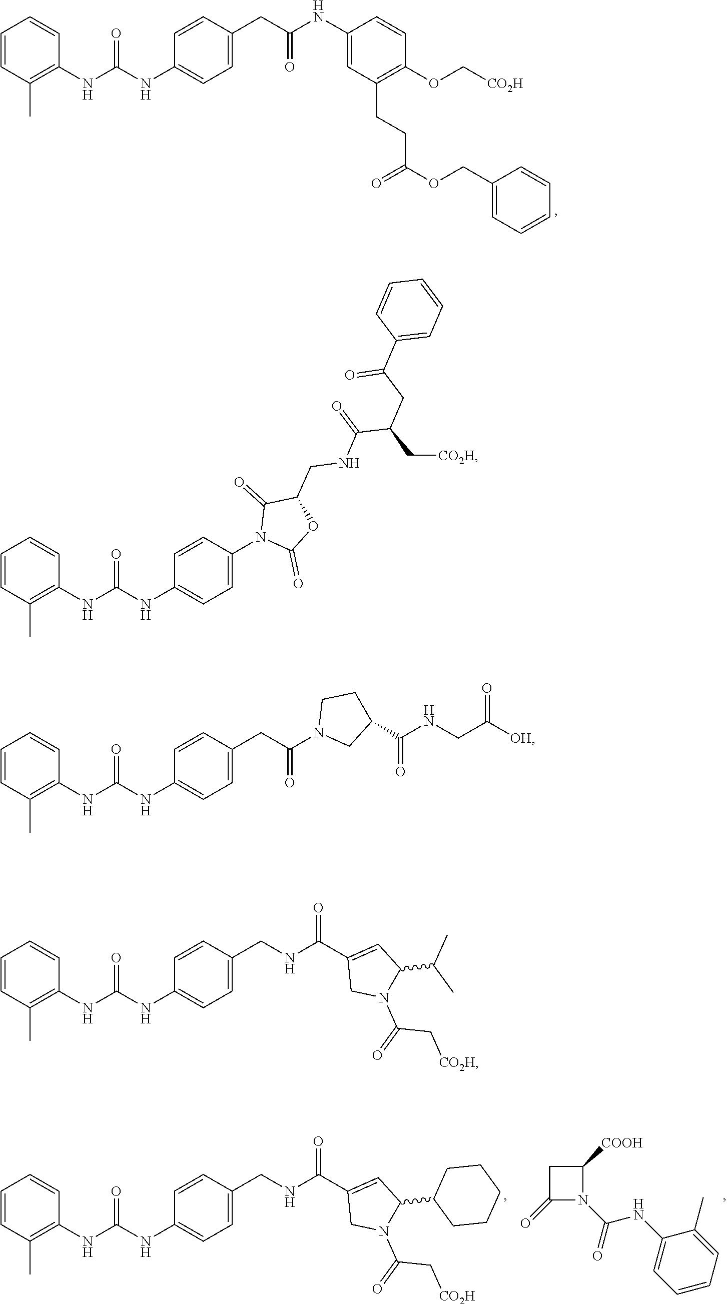

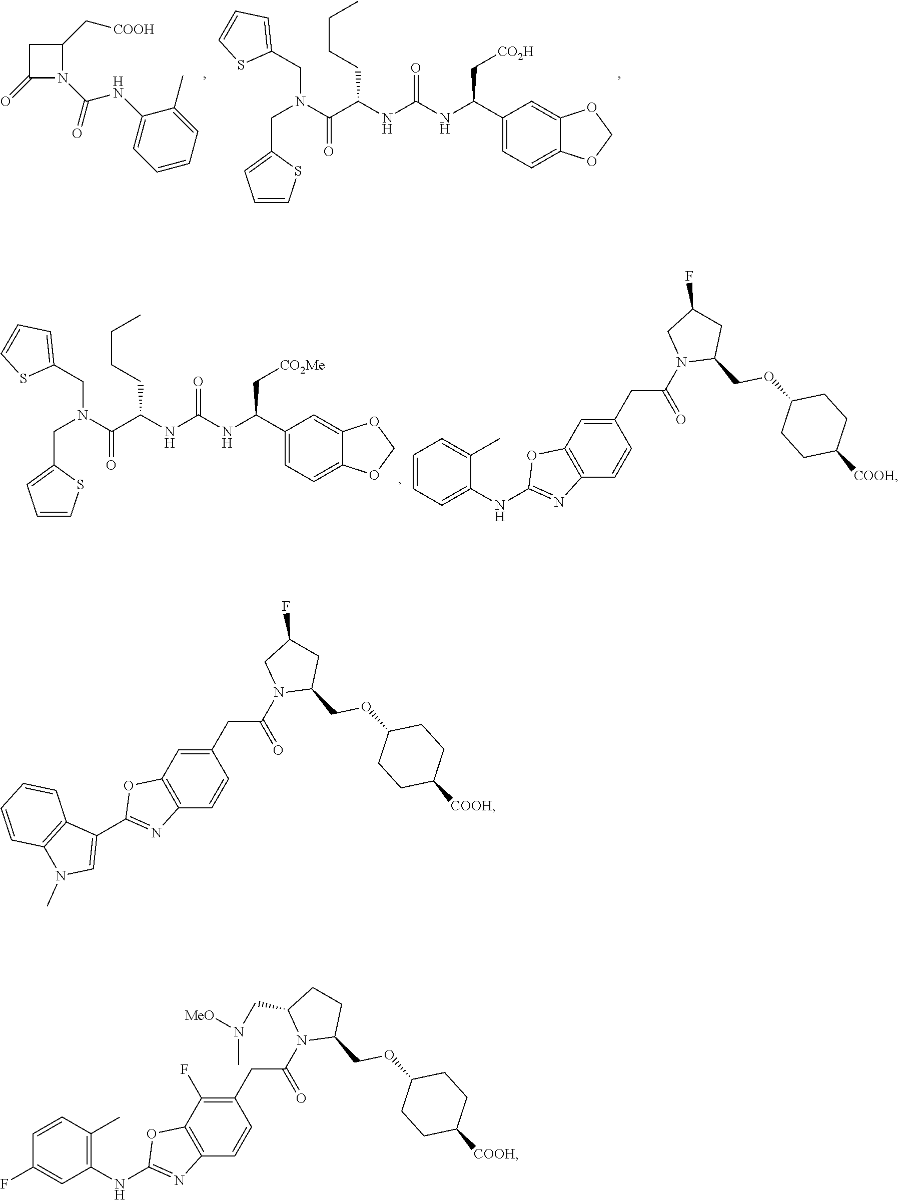

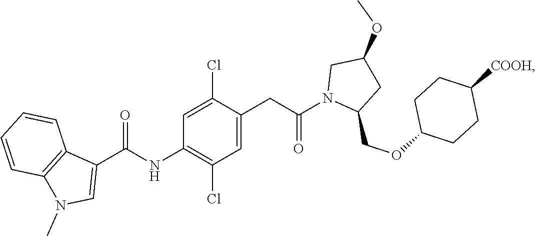 [SMILES: CO[C@H]1C[C@@H](CO[C@H]2CC[C@H](C(=O)O)CC2)N(C(=O)Cc2cc(Cl)c(NC(=O)c3cn(C)c4ccccc34)cc2Cl)C1]

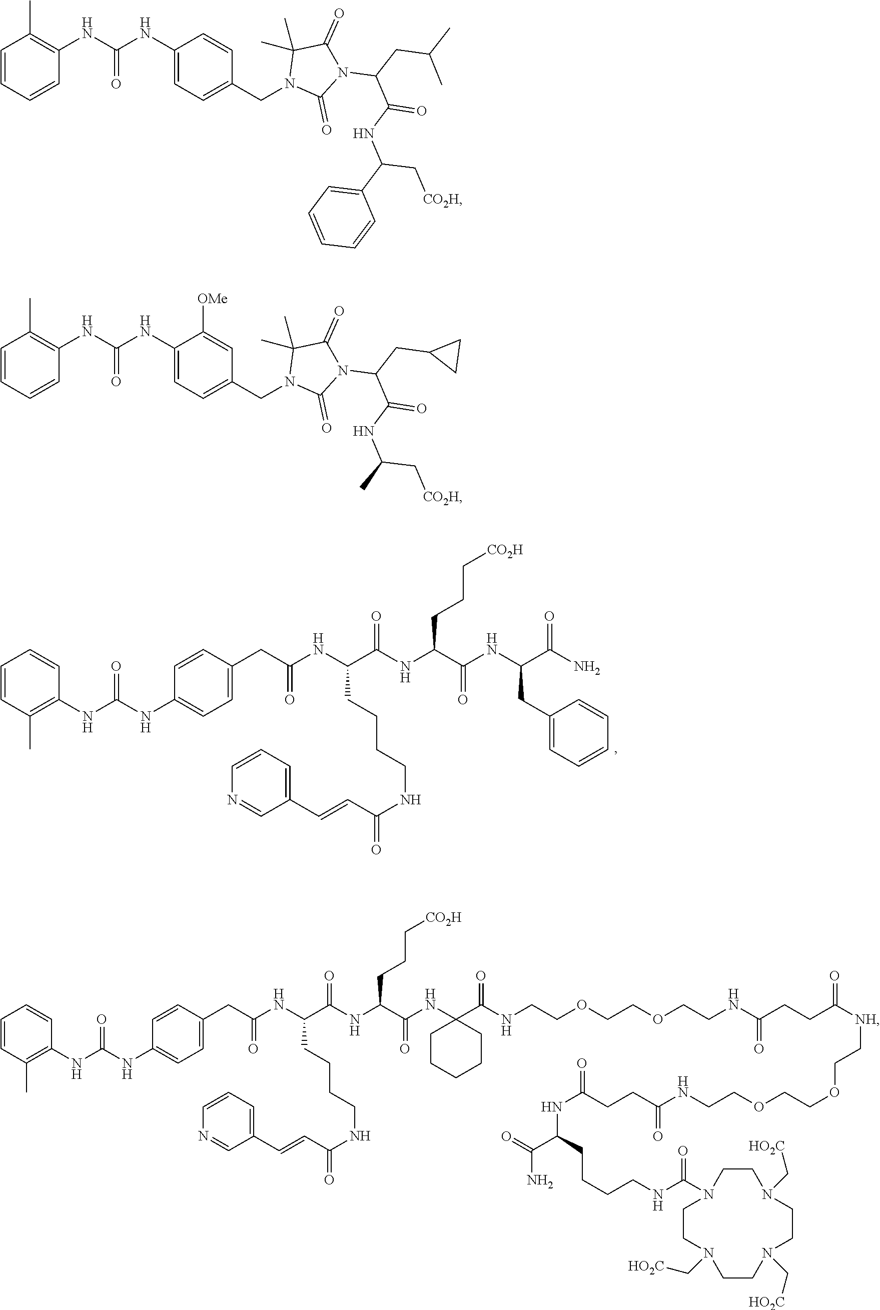

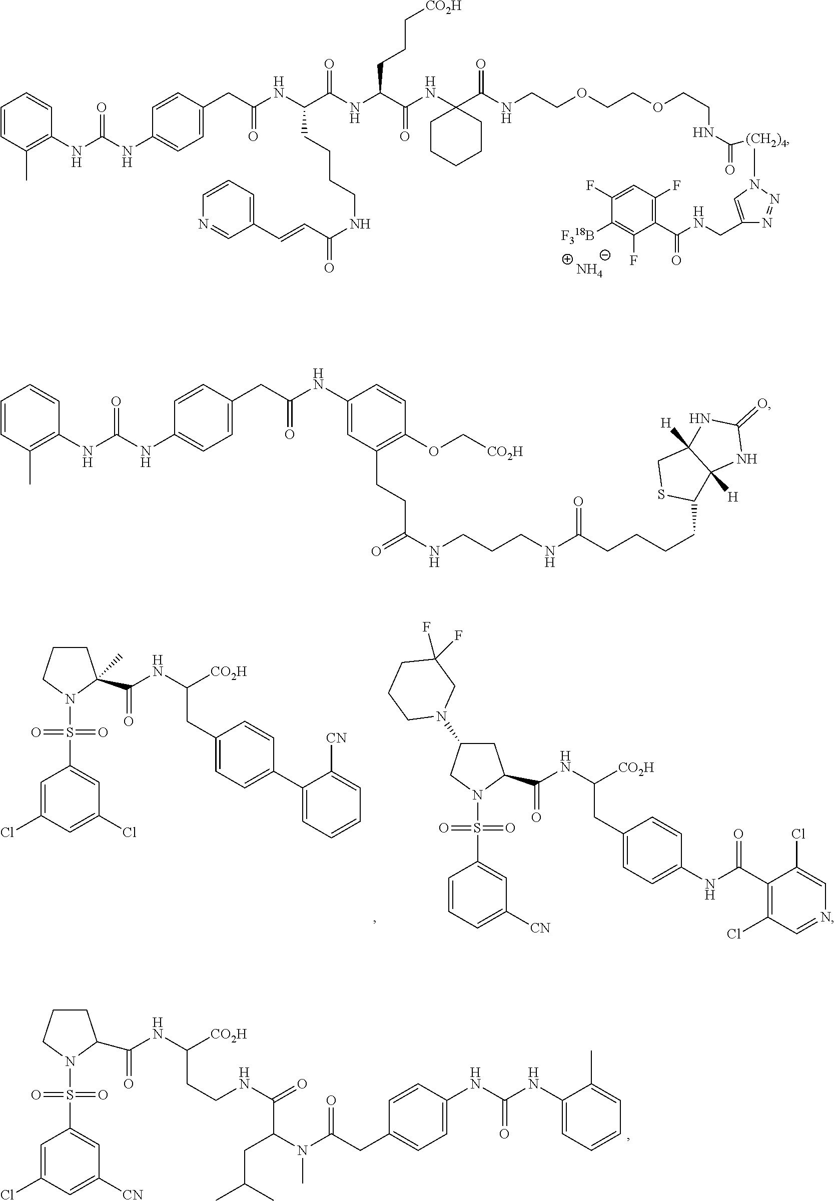

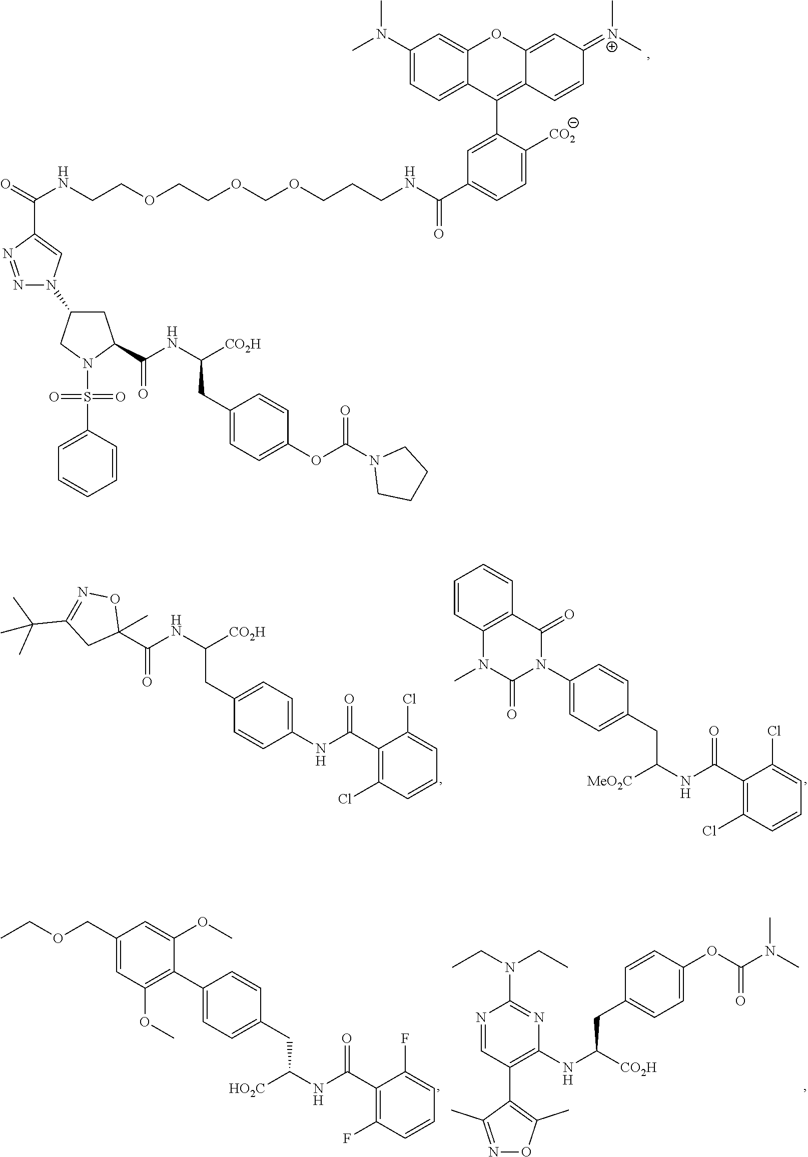

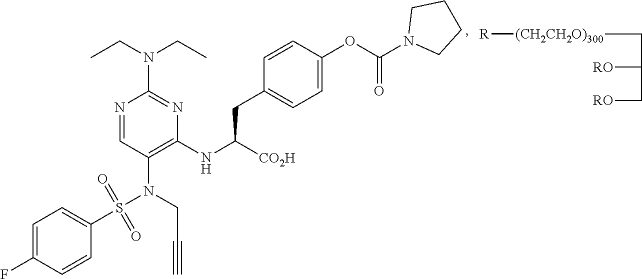

wherein each instance of

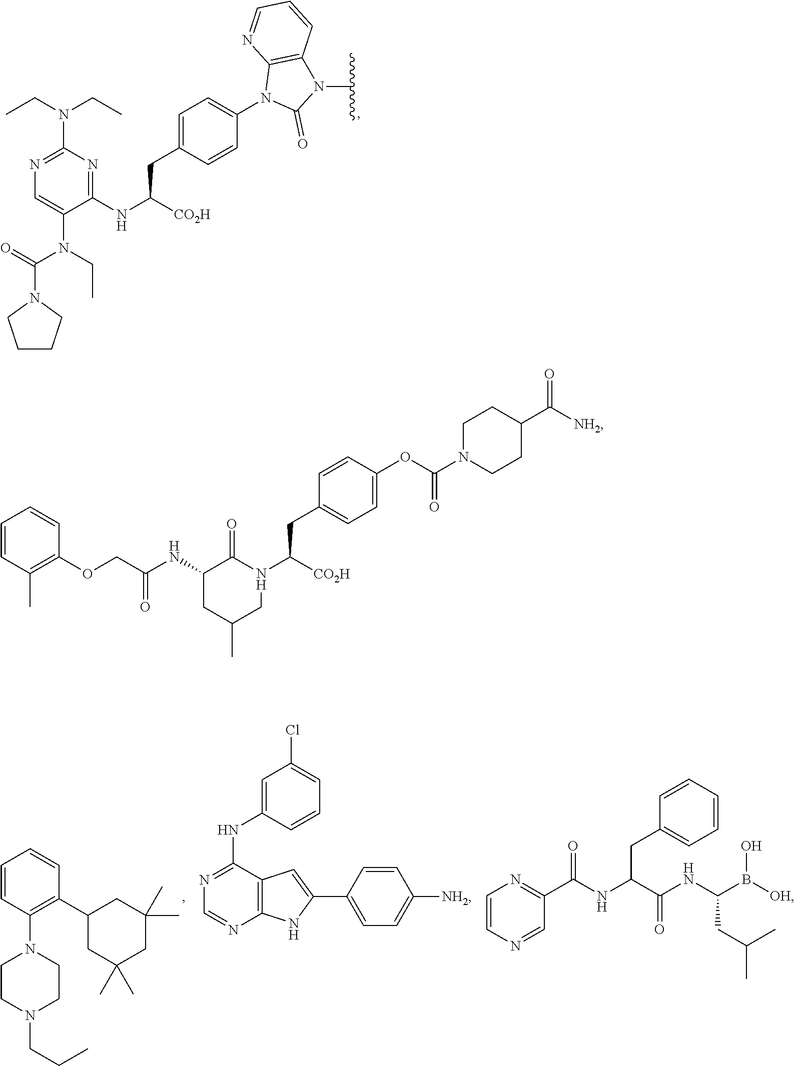

an anti-α₄β_{1/7} integrin antibody, and derivatives thereof.

Embodiment P5. The compound, or salt thereof, of embodiment 1, wherein the α₄β_{1/7} integrin ligand comprises the structure

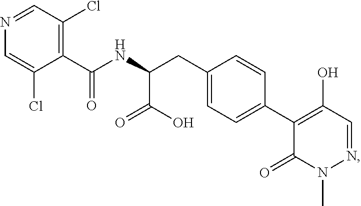 [SMILES: Cn1ncc(O)c(-c2ccc(C[C@H](NC(=O)c3c(Cl)cncc3Cl)C(=O)O)cc2)c1=O]

or a derivative thereof.

Embodiment P6. The compound, or salt thereof, of embodiment 1, wherein the compound comprises the structure of Formula (II):

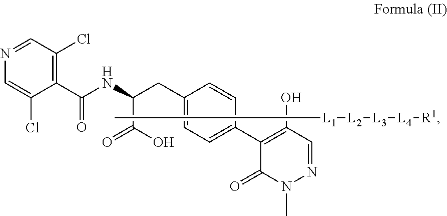

or a salt thereof.

Embodiment P7. The compound, or salt thereof, of embodiment 6, wherein the compound comprises the structure of Formula (II-a):

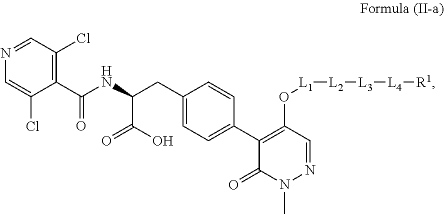

or a salt thereof.

Embodiment P8. The compound, or salt thereof, of any one of embodiments 1-7, wherein each of L₁, L₂, L₃, and L₄ is independently absent, a bond, an optionally substituted alkyl linker, an optionally substituted polyethylene glycol (PEG) linker, an optionally substituted heteroalkyl linker, an optionally substituted heteroaryl linker, a phosphodiester bond, or a phosphorothioate bond.

Embodiment P9. The compound, or salt thereof, of embodiment 8, wherein L₁ is a bond.

Embodiment P10. The compound, or salt thereof, of embodiment 8 or 9, wherein L₂ is an optionally substituted PEG linker.

Embodiment P11. The compound, or salt thereof, of embodiment 10, wherein the PEG linker is five PEG units in length.

Embodiment P12. The compound, or salt thereof, of any one of embodiments 8-11,

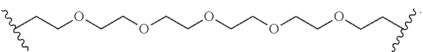 [SMILES: *CCOCCOCCOCCOCCOCC*]

wherein L₂ comprises the structure

Embodiment P13. The compound, or salt thereof, of any one of embodiments 8-12, wherein L₃ is an optionally substituted heteroaryl linker.

Embodiment P14. The compound, or salt thereof, of embodiment 13, wherein L₃ is an optionally substituted partially unsaturated heteroaryl linker.

Embodiment P15. The compound, or salt thereof, of embodiment 13 or 14, wherein L₃

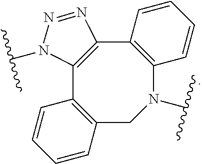 [SMILES: *N1Cc2ccccc2-c2c(nnn2*)-c2ccccc21]

comprises the structure

Embodiment P16. The compound, or salt thereof, of any one of embodiments 8-15, wherein L₄ is an optionally substituted heteroalkyl linker.

Embodiment P17. The compound, or salt thereof, of embodiment 16, wherein the heteroalkyl linker is substituted with one or more ═O substituents.

Embodiment P18. The compound, or salt thereof, of embodiment 16 or 17, wherein L₄

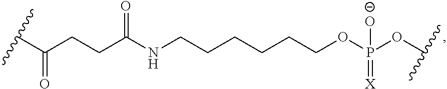

comprises the structure O H X wherein X is O or S.

Embodiment P19. The compound, or salt thereof, of any one of embodiments 8-18, wherein L₁, L₂, L₃, and L₄ together comprise the structure

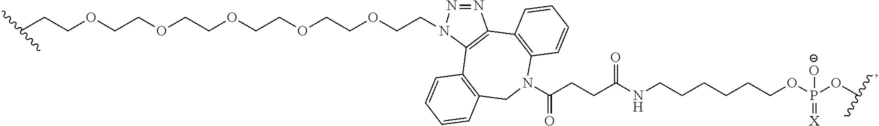

wherein X is O or S.

Embodiment P20. The compound, or salt thereof, of any one of embodiments 1-19, wherein the compound comprises the structure:

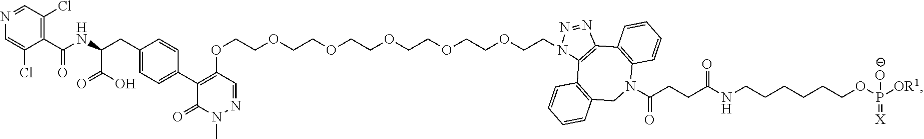

or a salt thereof, wherein X is O or S.

Embodiment P21. The compound, or salt thereof, of any one of embodiments 18-20, wherein X is O.

Embodiment P22. The compound, or salt thereof, of any one of embodiments 18-20, wherein X is S.

Embodiment P23. The compound, or salt thereof, of any one of embodiments 1-22, wherein R¹ comprises an oligonucleotide.

Embodiment P24. The compound, or salt thereof, of embodiment 23, wherein the oligonucleotide is attached at its 5′ end.

Embodiment P25. The compound, or salt thereof, of embodiment 23, wherein the oligonucleotide is attached at its 3′ end.

Embodiment P26. The compound, or salt thereof, of embodiment 23, wherein the oligonucleotide is attached at an internal position on the oligonucleotide.

Embodiment P27. The compound, or salt thereof, of embodiment 26, wherein the internal position is an internucleoside linkage.

Embodiment P28. The compound, or salt thereof, of any one of embodiments 1-27, wherein R¹ comprises an oligonucleotide conjugated to one or more additional α4β_{1/7} ligands.

Embodiment P29. The compound, or salt thereof, of embodiment 28, wherein the oligonucleotide is conjugated to two, three, four, five, or more than five additional α4β_{1/7} ligands.

Embodiment P30. The compound, or salt thereof, of embodiment 28 or 29, wherein the additional α₄β_{1/7} ligands are conjugated to the oligonucleotide at the 5′ end of the oligonucleotide, the 3′ end of the oligonucleotide, one or more internal positions on the oligonucleotide, or any combination thereof.

Embodiment P31. The compound, or salt thereof, of any one of embodiments 23-30, wherein the oligonucleotide is a modified oligonucleotide.

Embodiment P32. A composition comprising a compound, or salt thereof, of any one of embodiments 1-31, and a pharmaceutically acceptable excipient.

Embodiment P33. A method for delivering a therapeutic oligonucleotide to the brain of a subject, comprising administration of a compound, or salt thereof, of any one of embodiments 1-31, or a composition of embodiment 32, to the subject.

Embodiment P34. The method of embodiments 33, wherein the therapeutic oligonucleotide is delivered to one or more brain regions selected from the group consisting of the striatum, the cerebellum, the brain stem, the hippocampus, the frontal cortex, and the spinal cord.

Embodiment P35. A method for treating or ameliorating a disease, disorder, or symptom thereof in a subject, comprising administration of a compound, or salt thereof, of any one of embodiments 1-31, or a composition of embodiment 32, to the subject.

Embodiment P36. The method of embodiment 35, wherein the disease, disorder, or symptom thereof is a central nervous system (CNS) disease, disorder, or symptom thereof.

Embodiment P37. The method of embodiment 35 or 36, wherein the disease, disorder, or symptom thereof is Alzheimer's disease, or a symptom thereof.

Embodiment P38. The method of any one of embodiments 33-37, wherein the compound, or salt thereof, is administered to the subject intrathecally.

Embodiment P39. A method for making a compound, or salt thereof, of any one of embodiments 1-31, comprising one or more compounds and chemical transformations described herein, including Example 1.

Additional embodiments include embodiment 1 to embodiment 127 following:

Embodiment 1. A compound, or a stereoisomer, tautomer, prodrug, or salt thereof, comprising the structure of Formula (I′):

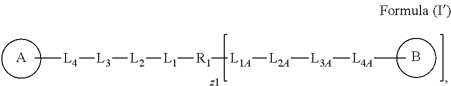

wherein:

each of

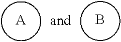

and is independently an α₄β_{1/7} integrin ligand;
each of L₁, L₂, L₃, L₄, L_{1A}, L_{2A}, L_{3A}, and L_{4A} is independently a linker, a bond, or absent;
R¹ comprises one or more oligonucleotides, protecting groups, small molecules, proteins, antibodies, and/or peptides; and
z1 is O or 1.

Embodiment 2. The compound, or a stereoisomer, tautomer, prodrug, or salt thereof, of embodiment 1, wherein the compound comprises the structure of Formula (I″):

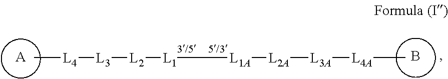

wherein:

[symbol] is an oligonucleotide.

Embodiment 3. The compound, or a stereoisomer, tautomer, prodrug, or salt thereof, of embodiment 1, wherein the compound comprises the structure of Formula (I):

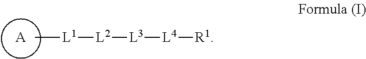

Embodiment 4. The compound, or a stereoisomer, tautomer, prodrug, or salt thereof, of any one of embodiments 1-3, wherein the α₄β_{1/7} integrin ligand is an α₄β_{1/7} integrin agonist.

Embodiment 5. The compound, or a stereoisomer, tautomer, prodrug, or salt thereof, of any one of embodiments 1-3, wherein the α₄β_{1/7} integrin ligand is an α₄β_{1/7} integrin antagonist.

Embodiment 6. The compound, or a stereoisomer, tautomer, prodrug, or salt thereof, of any one of embodiments 1-3, wherein the α₄β_{1/7} integrin ligand is selected from the group consisting of:

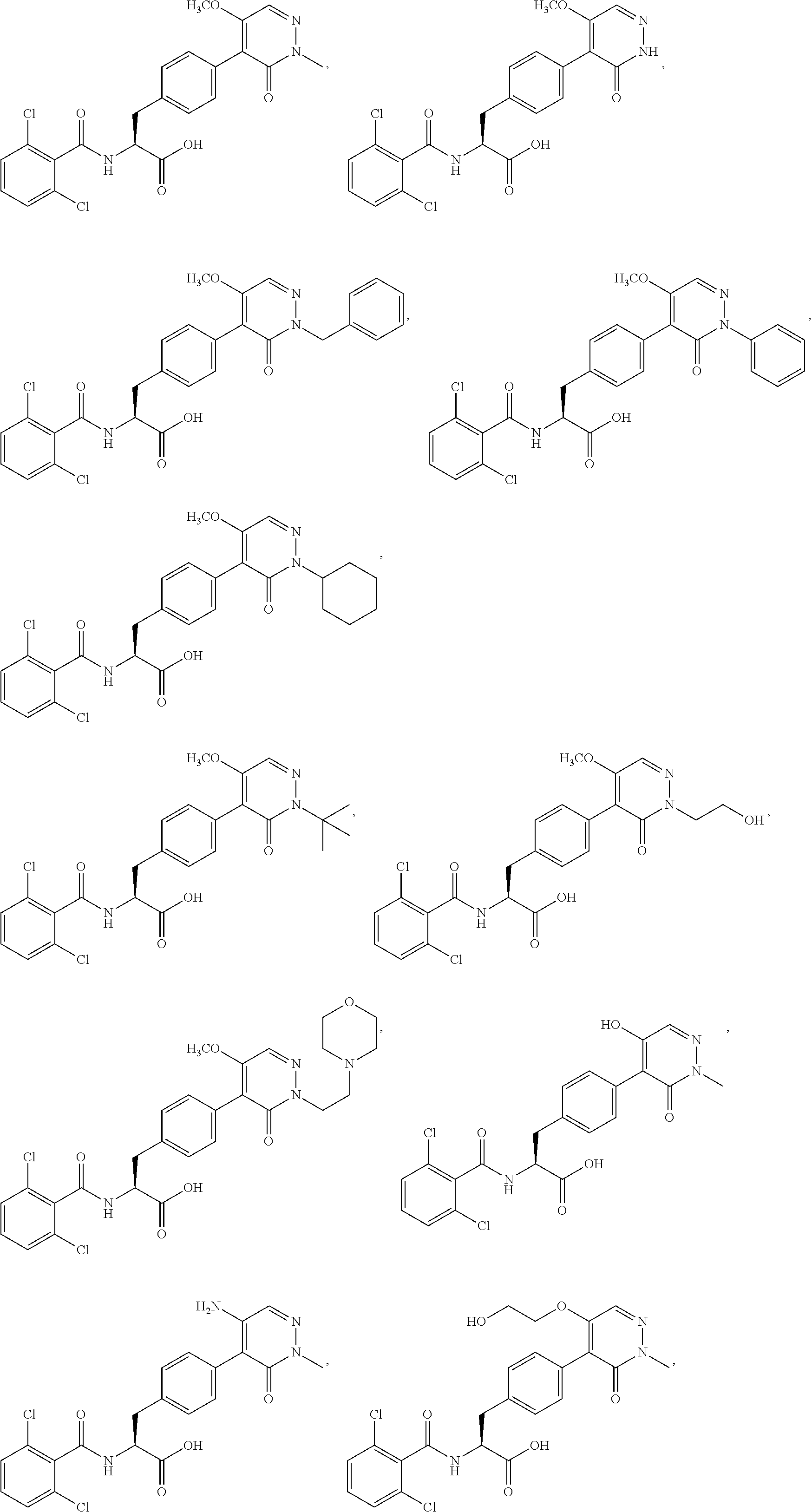

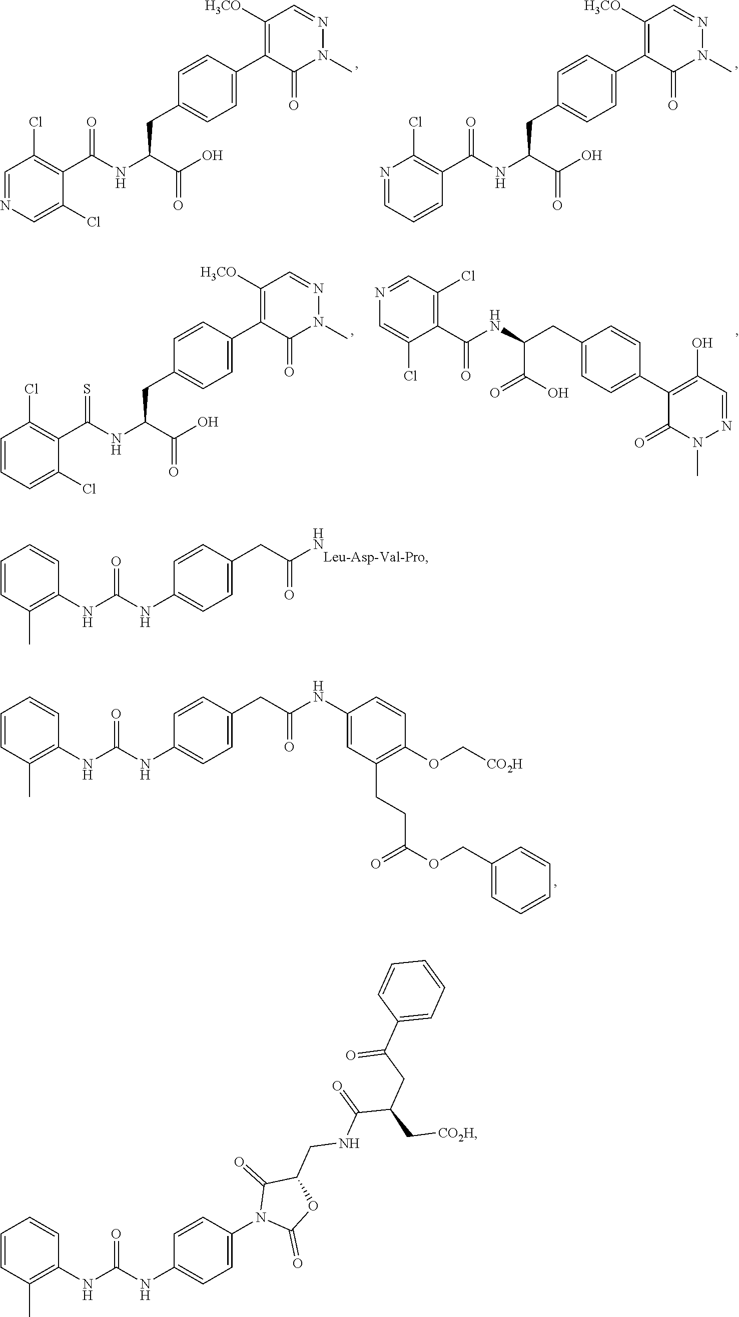

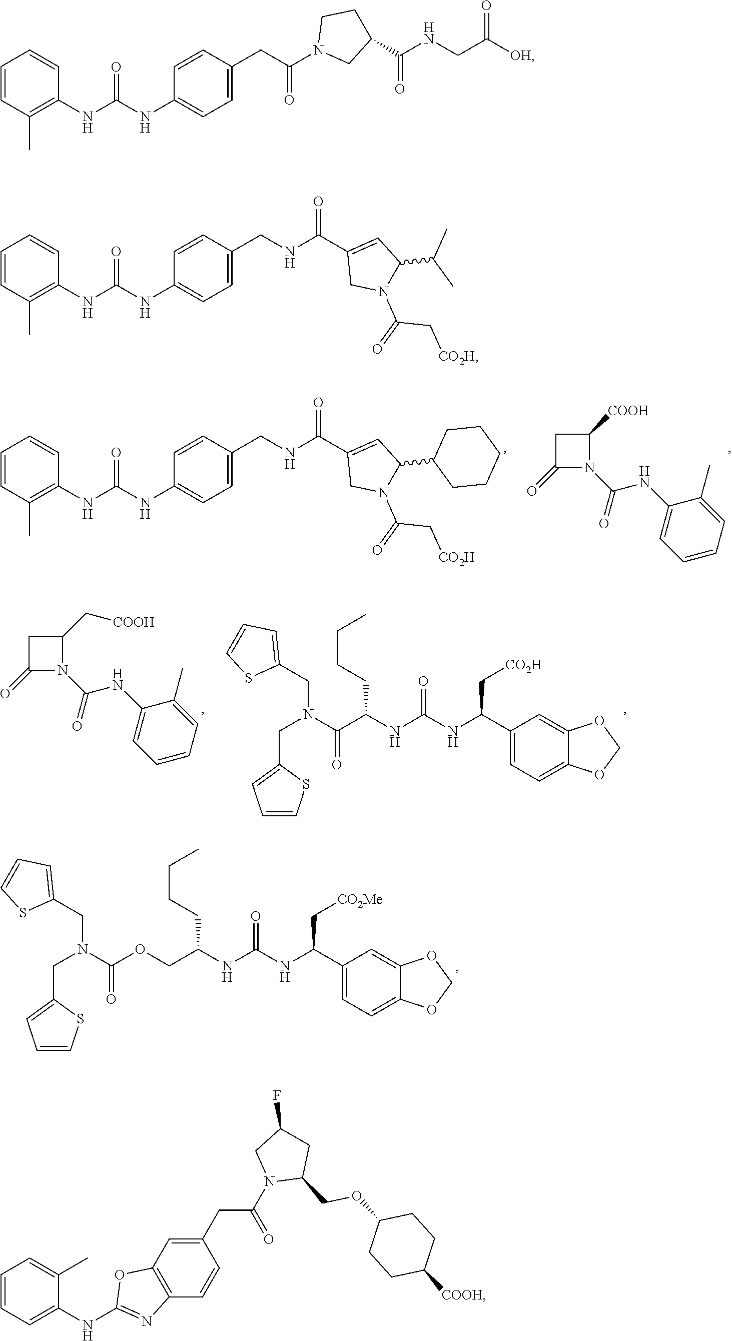

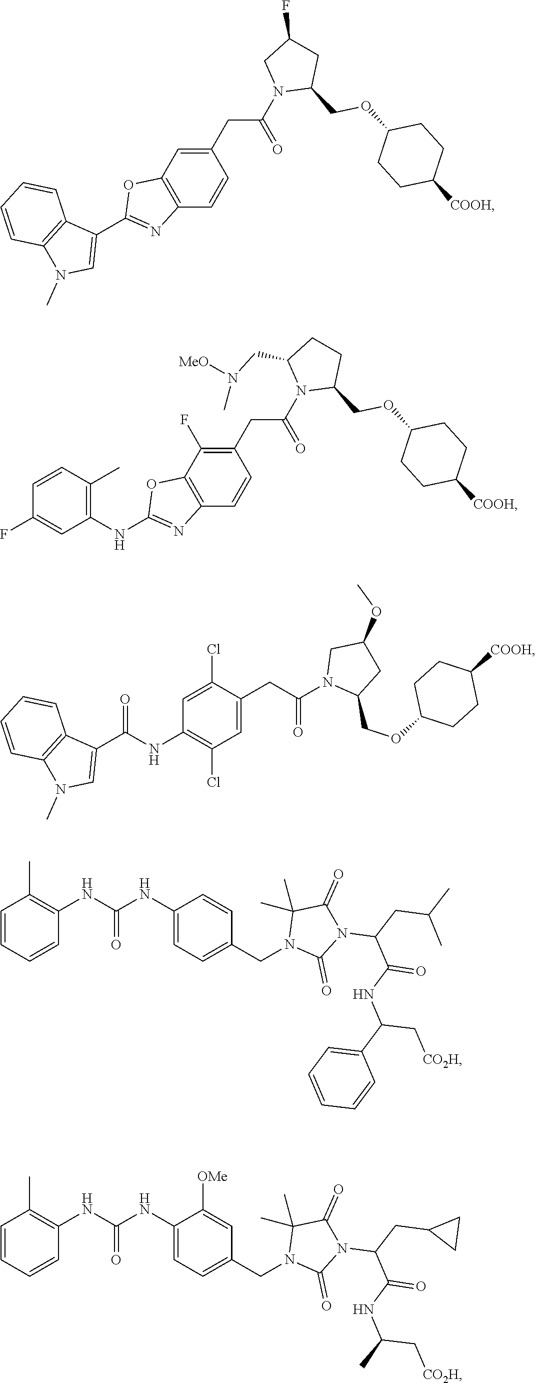

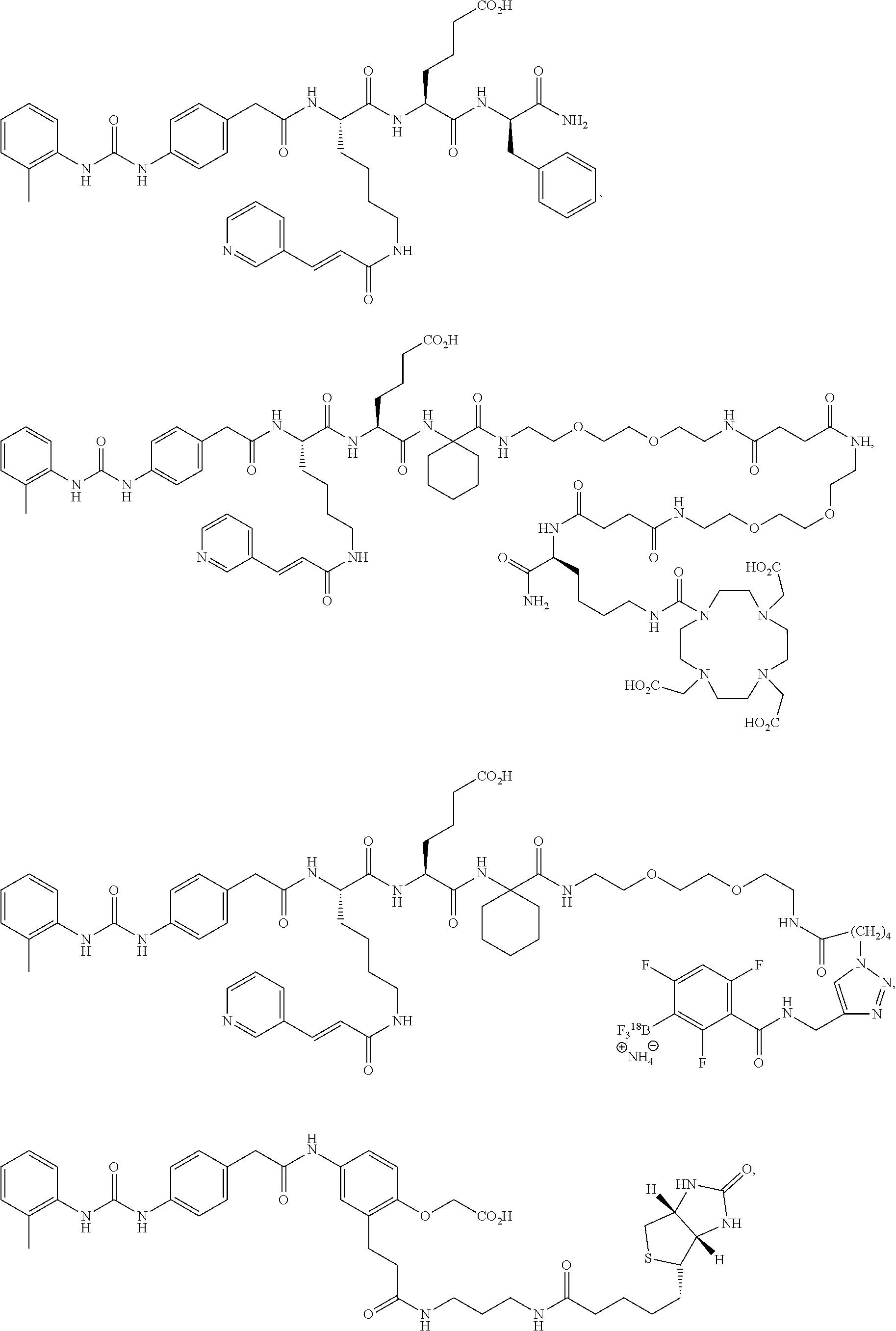

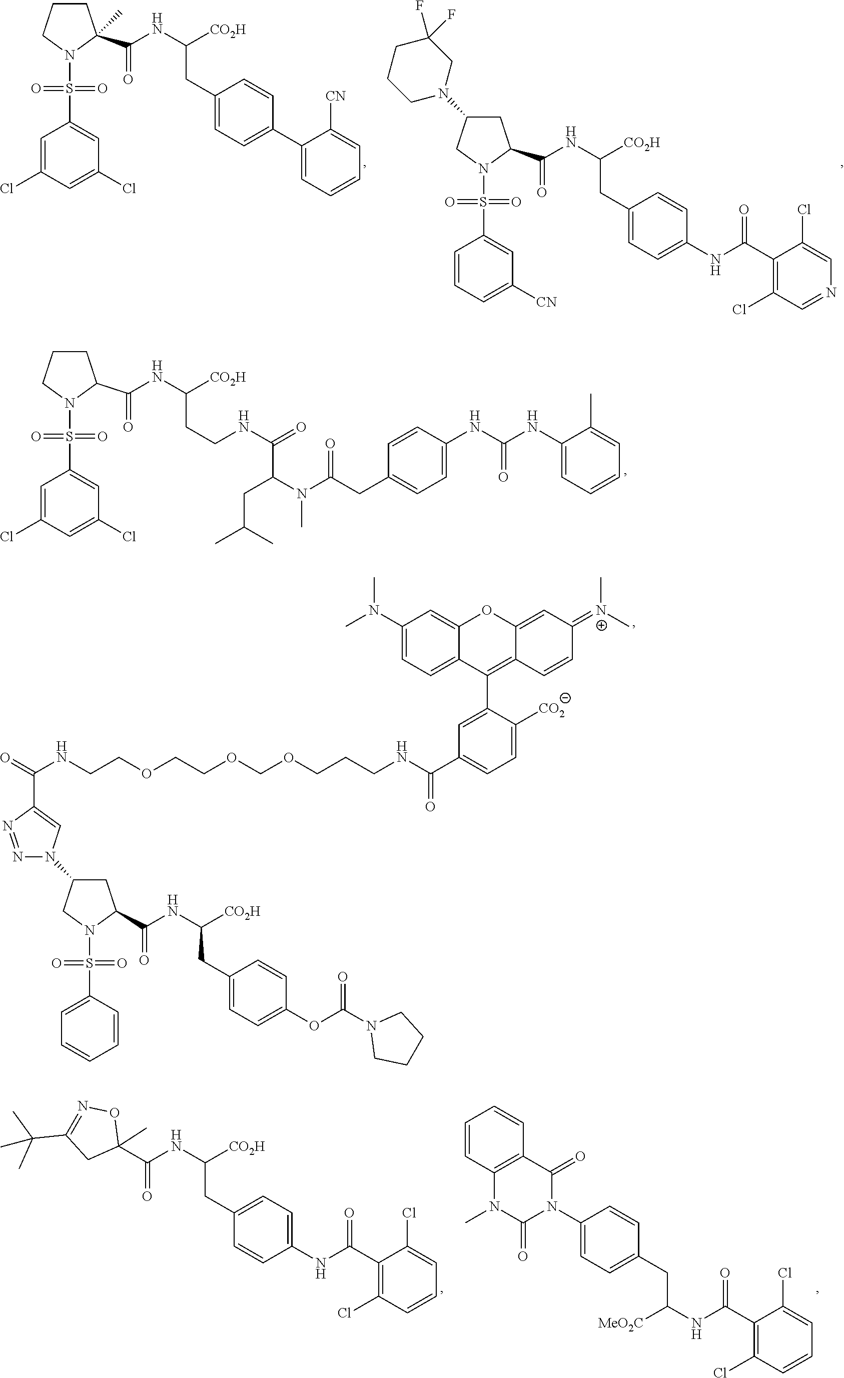

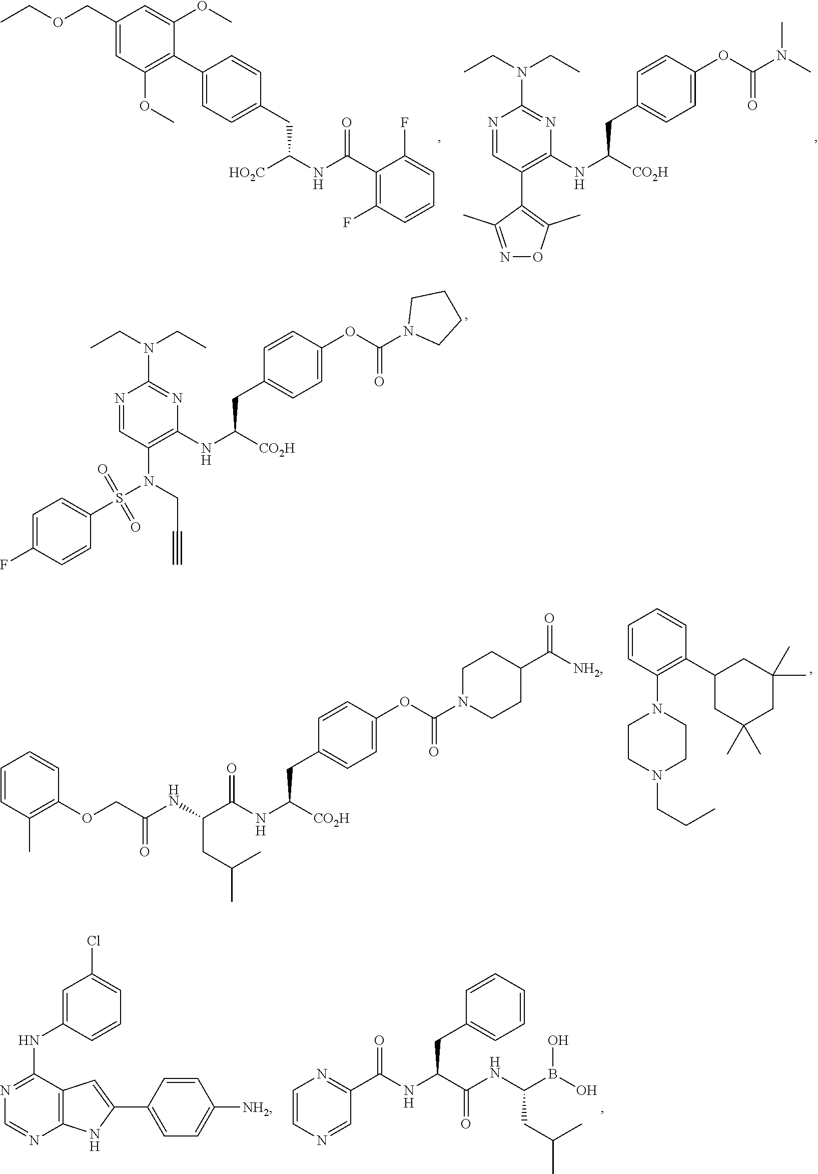

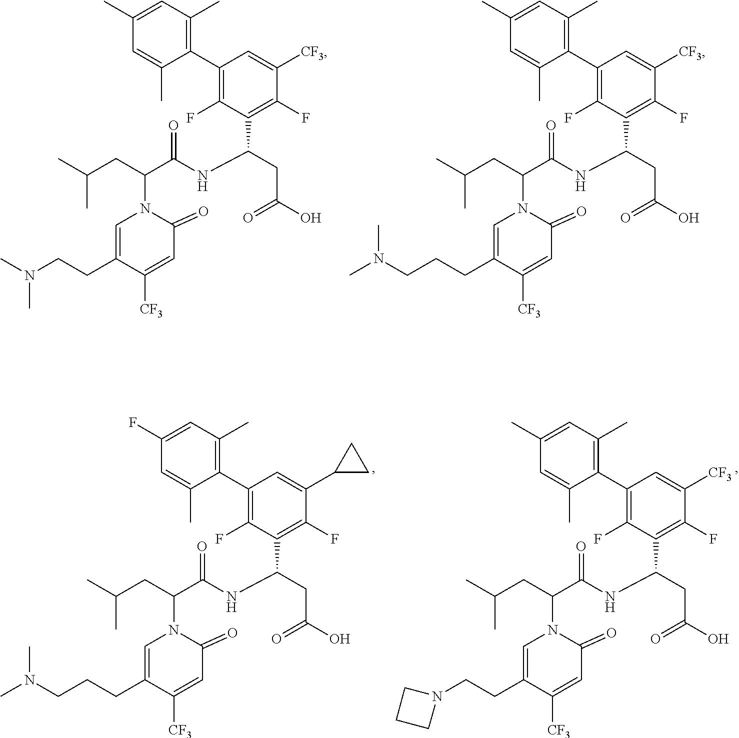 [SMILES: Cc1cc(C)c(-c2cc(C(F)(F)F)c(F)c([C@H](CC(=O)O)NC(=O)C(CC(C)C)n3cc(CCN(C)C)c(C(F)(F)F)cc3=O)c2F)c(C)c1 ; Cc1cc(C)c(-c2cc(C(F)(F)F)c(F)c([C@H](CC(=O)O)NC(=O)C(CC(C)C)n3cc(CCCN(C)C)c(C(F)(F)F)cc3=O)c2F)c(C)c1 ; Cc1cc(F)cc(C)c1-c1cc(C2CC2)c(F)c([C@H](CC(=O)O)NC(=O)C(CC(C)C)n2cc(CCCN(C)C)c(C(F)(F)F)cc2=O)c1F ; Cc1cc(C)c(-c2cc(C(F)(F)F)c(F)c([C@H](CC(=O)O)NC(=O)C(CC(C)C)n3cc(CCN4CCC4)c(C(F)(F)F)cc3=O)c2F)c(C)c1]

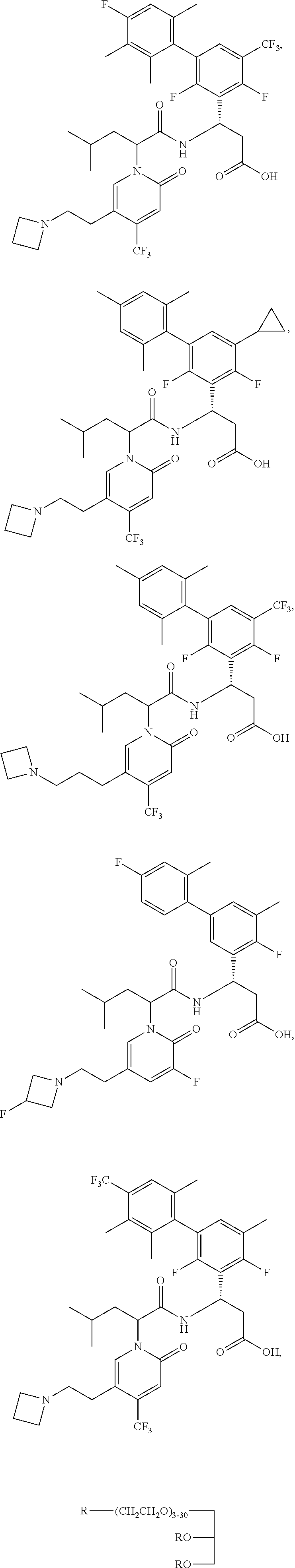

wherein each instance of R is

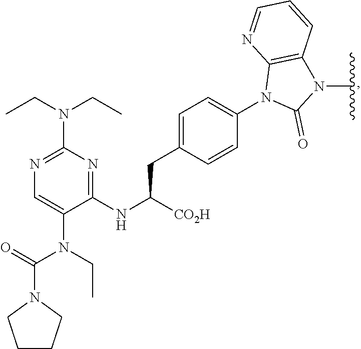 [SMILES: *n1c(=O)n(-c2ccc(C[C@H](Nc3nc(N(CC)CC)ncc3N(CC)C(=O)N3CCCC3)C(=O)O)cc2)c2ncccc21]

an anti-α₄β_{1/7} integrin antibody, and derivatives thereof.

Embodiment 7. The compound, or a stereoisomer, tautomer, prodrug, or salt thereof, of embodiment 1, wherein the compound comprises the structure of Formula (II′):

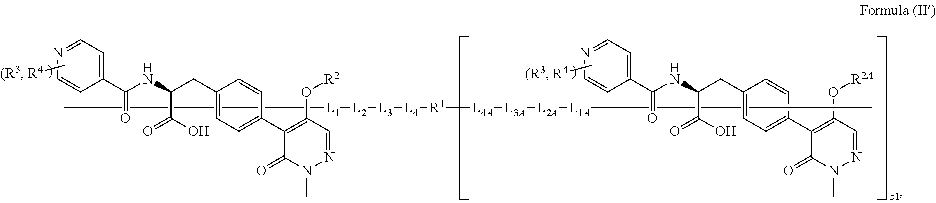

wherein
R² and R^{2A} are each independently H, polyethylene glycol (PEG), optionally substituted heteroalkyl, or optionally substituted heteroaryl; and
R³, R^{3A}, R⁴, and R^{4A} are each independently H, halogen, optionally substituted alkyl, or optionally substituted —O-alkyl.

Embodiment 8. The compound, or a stereoisomer, tautomer, prodrug, or salt thereof, of embodiment 7, wherein the compound comprises the structure of Formula (II″):

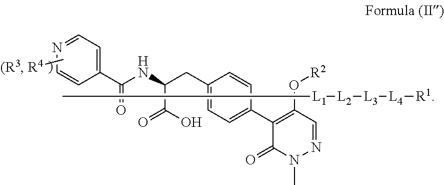

Embodiment 9. The compound, or a stereoisomer, tautomer, prodrug, or salt thereof, of embodiment 8, wherein the compound comprises the structure of Formula (II″-a):

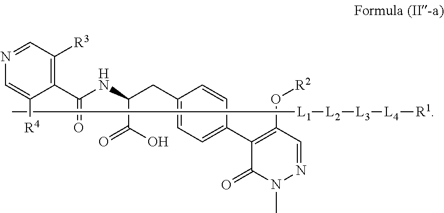

Embodiment 10. The compound, or a stereoisomer, tautomer, prodrug, or salt thereof, of embodiment 9, wherein the compound comprises the structure of Formula (II″-a-1):

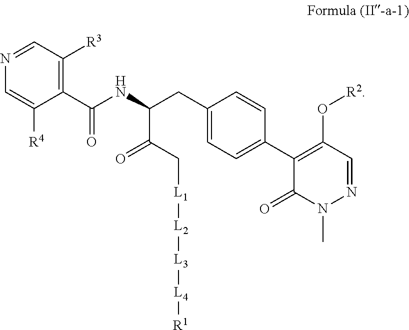

Embodiment 11. The compound, or a stereoisomer, tautomer, prodrug, or salt thereof, of embodiment 9, wherein the compound comprises the structure of Formula (II″-a-2):

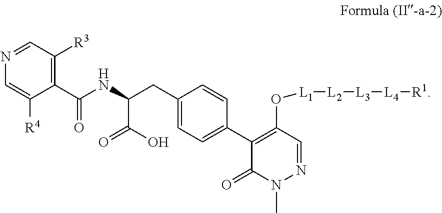

Embodiment 12. The compound, or a stereoisomer, tautomer, prodrug, or salt thereof, of any one of embodiments 1-11, wherein the α₄β_{1/7} integrin ligand comprises the structure

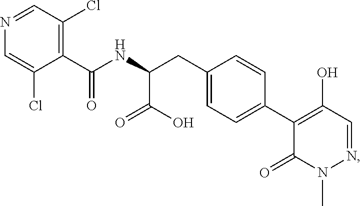 [SMILES: Cn1ncc(O)c(-c2ccc(C[C@H](NC(=O)c3c(Cl)cncc3Cl)C(=O)O)cc2)c1=O]

or a derivative thereof.

Embodiment 13. The compound, or a stereoisomer, tautomer, prodrug, or salt thereof, of embodiment 8, wherein the compound comprises the structure of Formula (II):

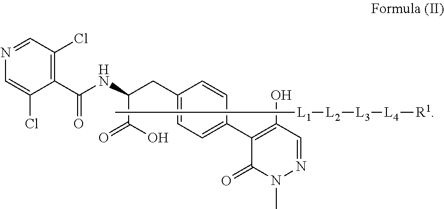

Embodiment 14. The compound, or a stereoisomer, tautomer, prodrug, or salt thereof, of embodiment 13, wherein the compound comprises the structure of Formula (II-a):

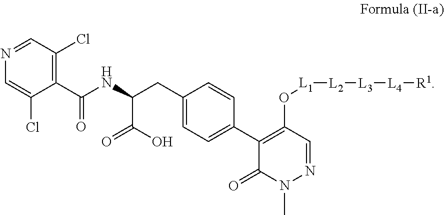

Embodiment 15. The compound, or a stereoisomer, tautomer, prodrug, or salt thereof, of embodiment 1, wherein the compound comprises the structure of Formula (III′):

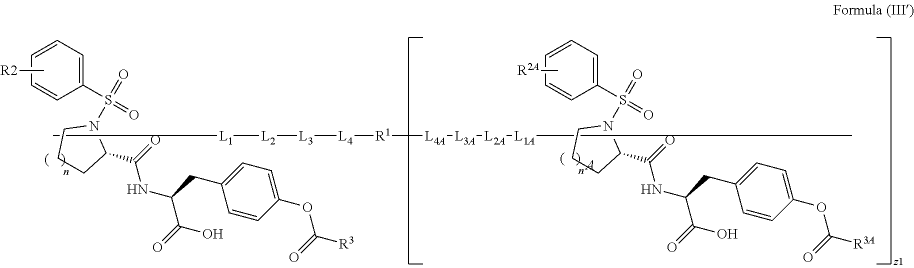

wherein
R² and R^{2A} are each independently H, halogen, polyethylene glycol (PEG), optionally substituted alkyl, optionally substituted heteroalkyl, optionally substituted heteroaryl, optionally substituted —O-alkyl, or optionally substituted cycloalkyl;
R³ and R^{3A} are each independently optionally substituted heteroalkyl or optionally substituted heterocyclyl; and
n and nA are each independently 1, 2, or 3.

Embodiment 16. The compound, or a stereoisomer, tautomer, prodrug, or salt thereof, of embodiment 15, wherein the compound comprises the structure of Formula (III):

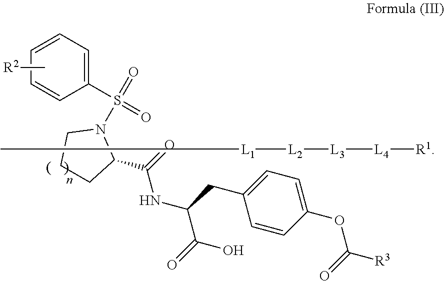

Embodiment 17. The compound, or a stereoisomer, tautomer, prodrug, or salt thereof, of embodiment 16, wherein the compound comprises the structure of Formula (III-a):

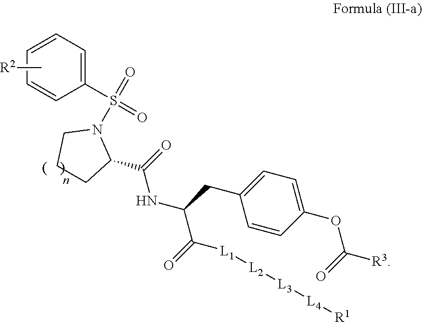

Embodiment 18. The compound, or a stereoisomer, tautomer, prodrug, or salt thereof, of embodiment 16, wherein the compound comprises the structure of Formula (III-b):

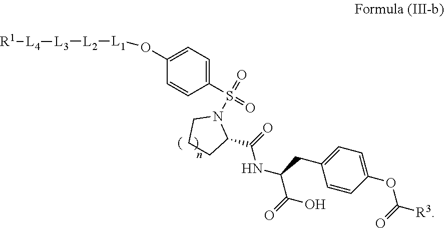

Embodiment 19. The compound, or a stereoisomer, tautomer, prodrug, or salt thereof, of embodiment 1, wherein the compound comprises the structure of Formula (IV′):

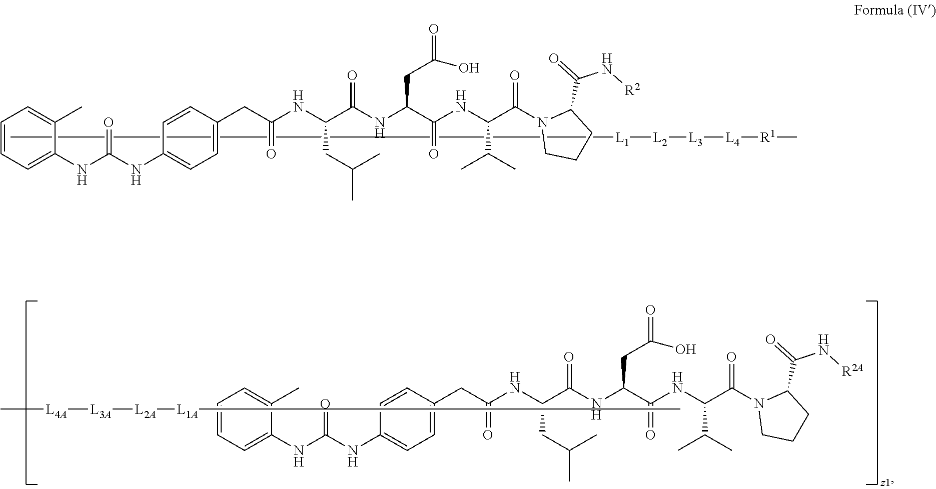

wherein
R² and R^{2A} are each independently H, —OH, —NH₂, —NHR³, —OR³, or absent; and each instance of R³ is independently H, polyethylene glycol, optionally substituted alkyl, optionally substituted heteroalkyl, optionally substituted cycloalkyl, or optionally substituted heteroaryl.

Embodiment 20. The compound, or a stereoisomer, tautomer, prodrug, or salt thereof, of embodiment 19, wherein the compound comprises the structure of Formula (IV):

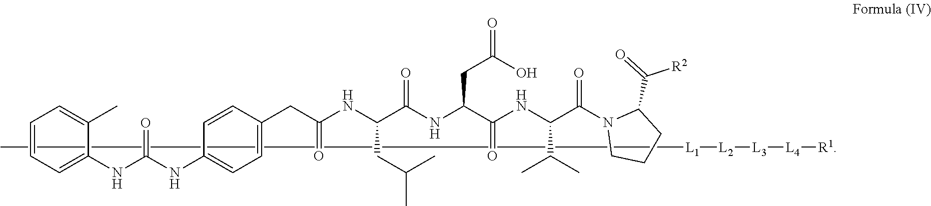

Embodiment 21. The compound, or a stereoisomer, tautomer, prodrug, or salt thereof, of embodiment 20, wherein the compound comprises the structure of Formula (IV-a):

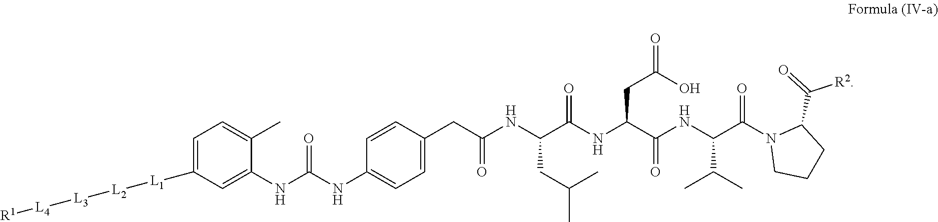

Embodiment 22. The compound, or a stereoisomer, tautomer, prodrug, or salt thereof, of embodiment 20, wherein the compound comprises the structure of Formula (IV-b):

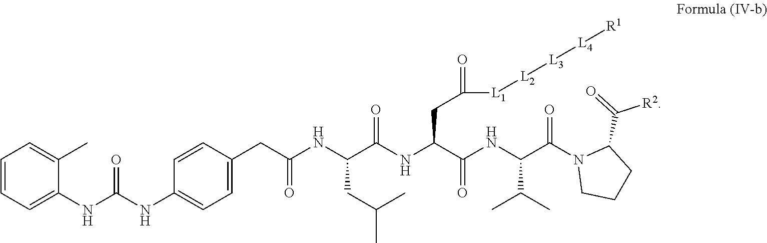

Embodiment 23. The compound, or a stereoisomer, tautomer, prodrug, or salt thereof, of embodiment 20, wherein the compound comprises the structure of Formula (IV-c):

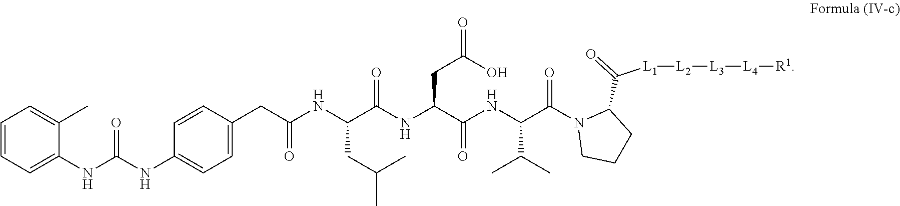

Embodiment 24. The compound, or a stereoisomer, tautomer, prodrug, or salt thereof, of embodiment 1, wherein the compound comprises the structure of Formula (V′):

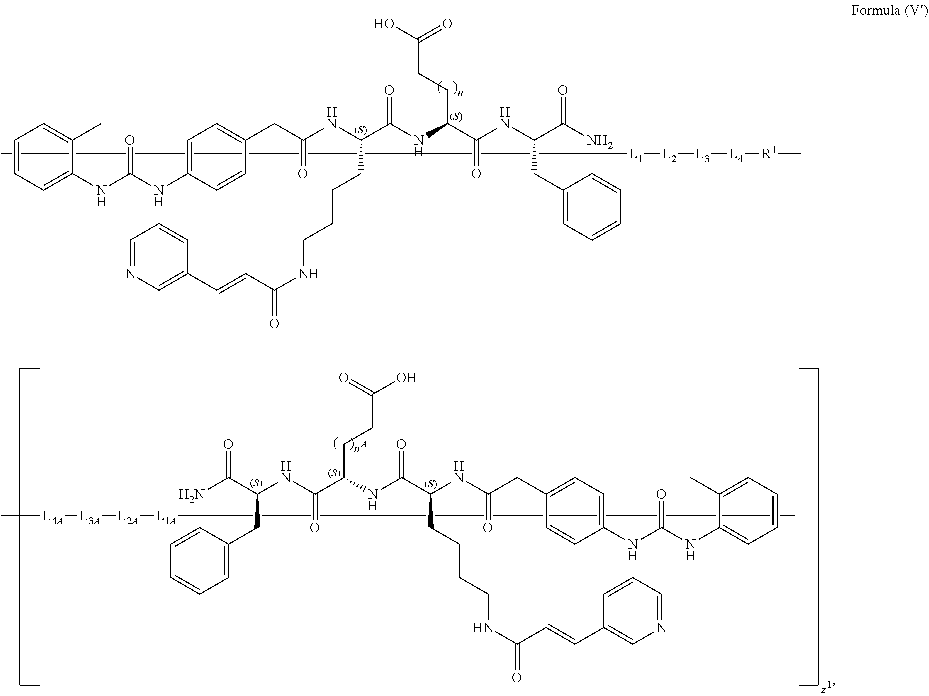

wherein
n and n^{A} are each independently 0, 1, 2, or, 3.

Embodiment 25. The compound, or a stereoisomer, tautomer, prodrug, or salt thereof, of embodiment 24, wherein the compound comprises the structure of Formula (V′-a):

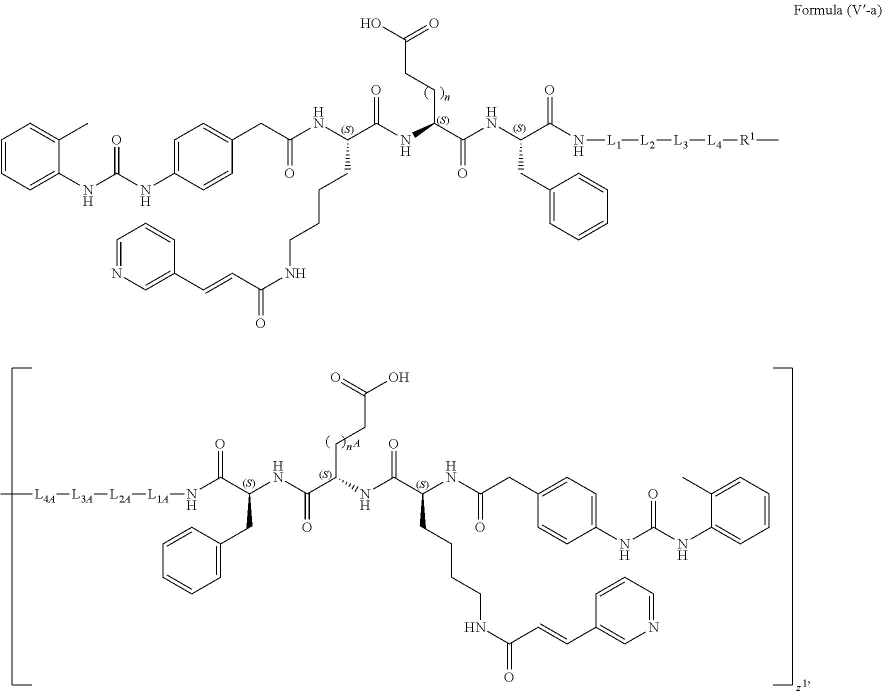

Embodiment 26. The compound, or a stereoisomer, tautomer, prodrug, or salt thereof, of embodiment 24, wherein the compound comprises the structure of Formula (V):

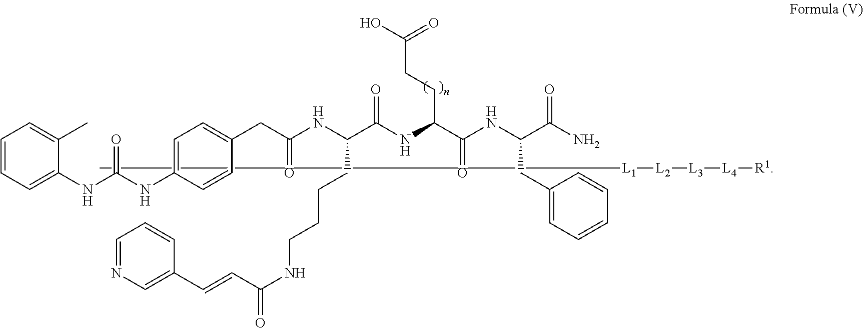

Embodiment 27. The compound, or a stereoisomer, tautomer, prodrug, or salt thereof, of embodiment 26, wherein the compound comprises the structure of Formula (V-a):

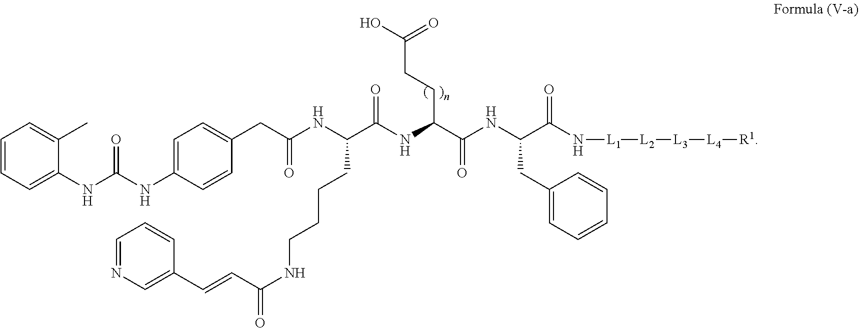

Embodiment 28. The compound, or a stereoisomer, tautomer, prodrug, or salt thereof, of embodiment 26, wherein the compound comprises the structure of Formula (V-b):

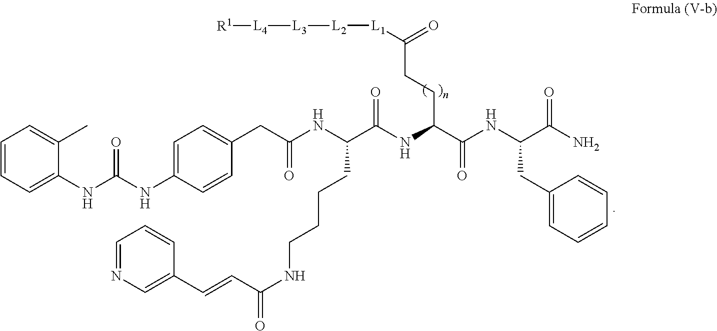

Embodiment 29. The compound, or a stereoisomer, tautomer, prodrug, or salt thereof, of embodiment 26, wherein the compound comprises the structure of Formula (V-c):

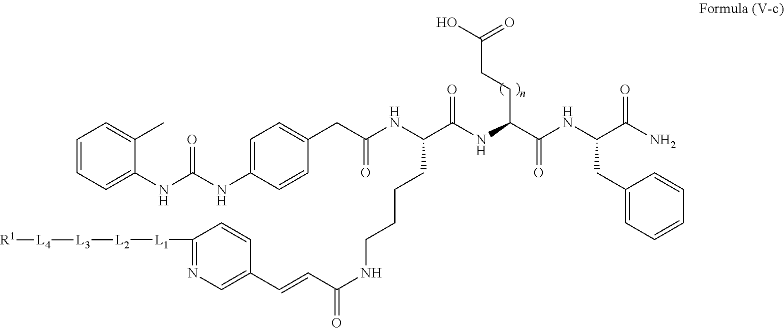

Embodiment 30. The compound, or a stereoisomer, tautomer, prodrug, or salt thereof, of embodiment 26, wherein the compound comprises the structure of Formula (V-d):

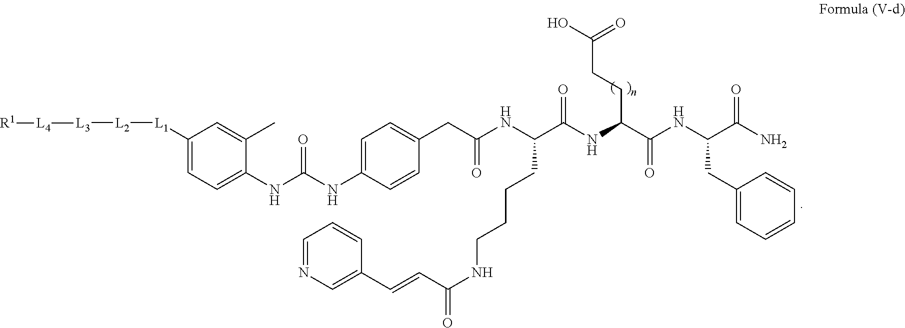

Embodiment 31. The compound, or a stereoisomer, tautomer, prodrug, or salt thereof, of embodiment 26, wherein the compound comprises the structure of Formula (V-e):

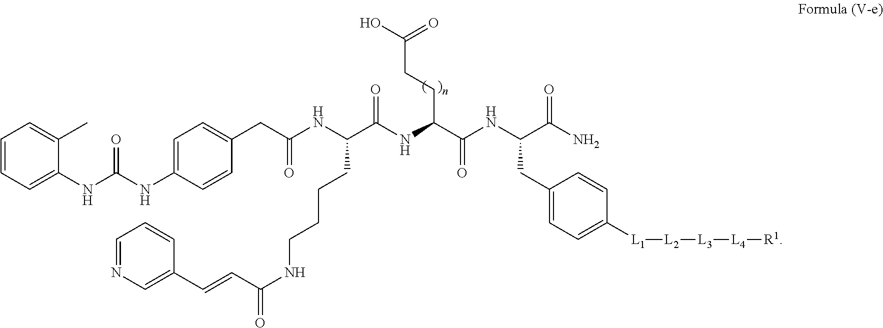

Embodiment 32. The compound, or a stereoisomer, tautomer, prodrug, or salt thereof, of embodiment 1, wherein the compound comprises the structure of Formula (VI′):

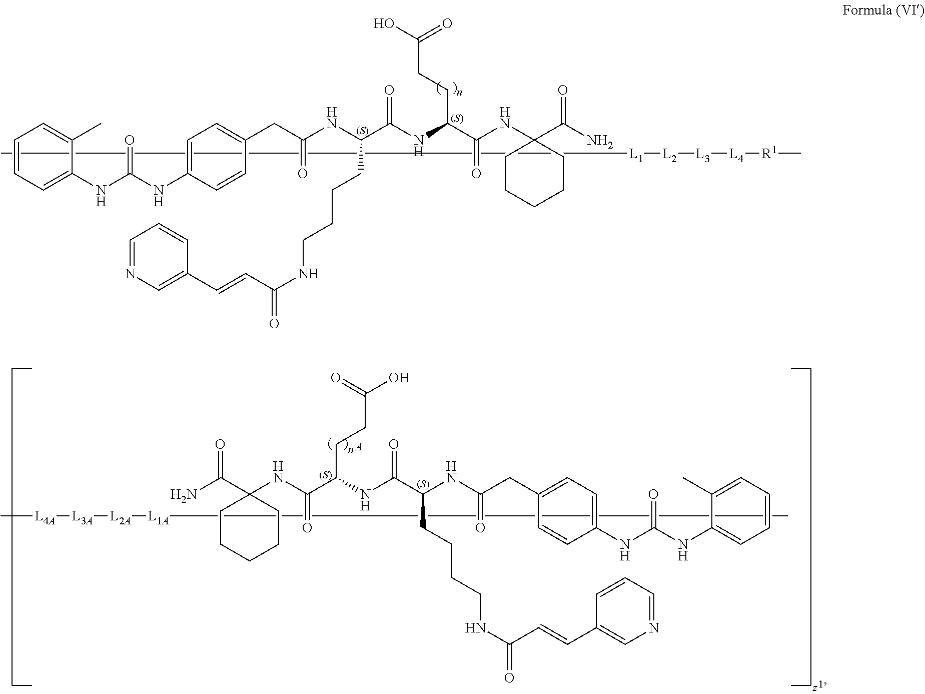

wherein
n and nA are each independently 0, 1, 2, or 3.

Embodiment 33. The compound, or a stereoisomer, tautomer, prodrug, or salt thereof, of embodiment 32, wherein the compound comprises the structure of Formula (VI′-a):

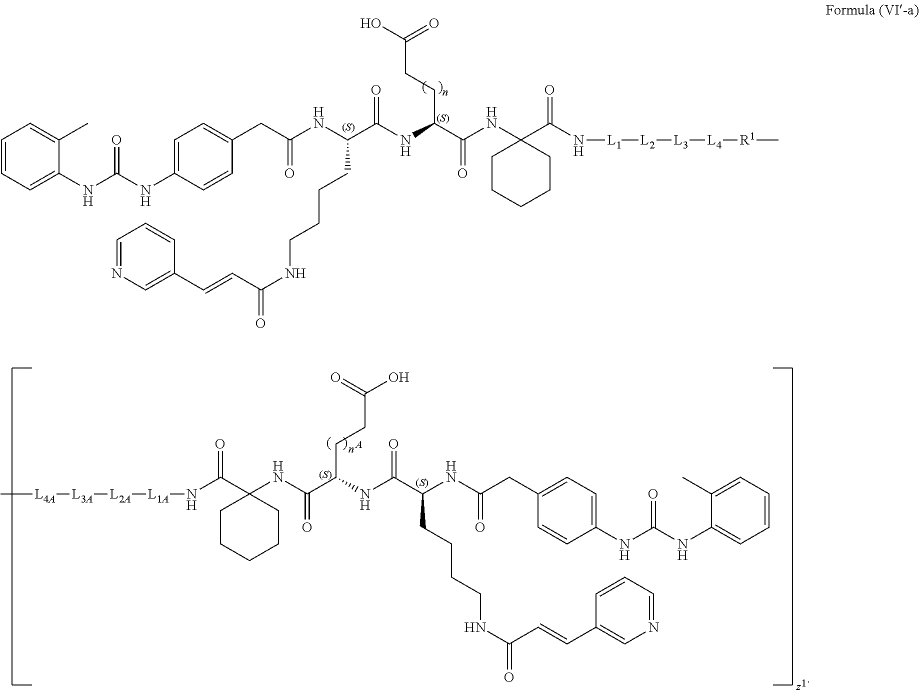

Embodiment 34. The compound, or a stereoisomer, tautomer, prodrug, or salt thereof, of embodiment 32, wherein the compound comprises the structure of Formula (VI):

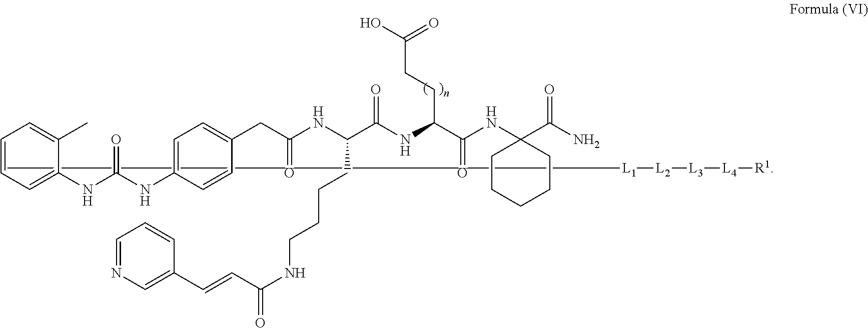

Embodiment 35. The compound, or a stereoisomer, tautomer, prodrug, or salt thereof, of embodiment 34, wherein the compound comprises the structure of Formula (VI-a):

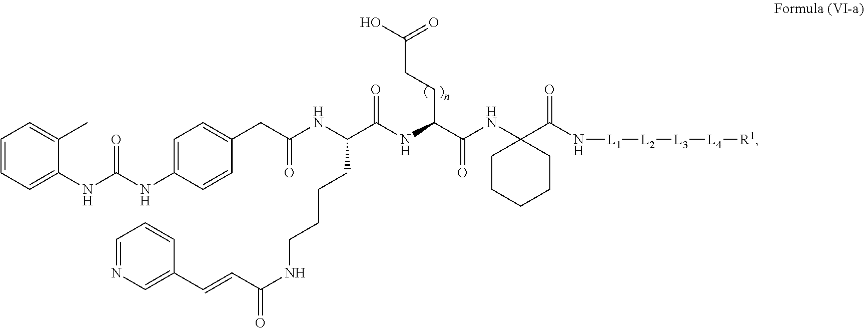

Embodiment 36. The compound, or a stereoisomer, tautomer, prodrug, or salt thereof, of embodiment 34, wherein the compound comprises the structure of Formula (VI-b):

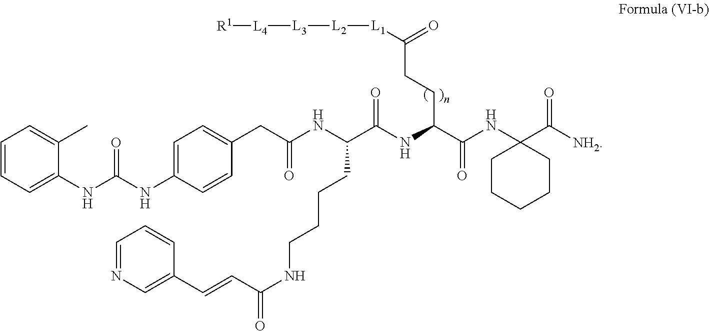

Embodiment 37. The compound, or a stereoisomer, tautomer, prodrug, or salt thereof, of embodiment 34, wherein the compound comprises the structure of Formula (VI-c):

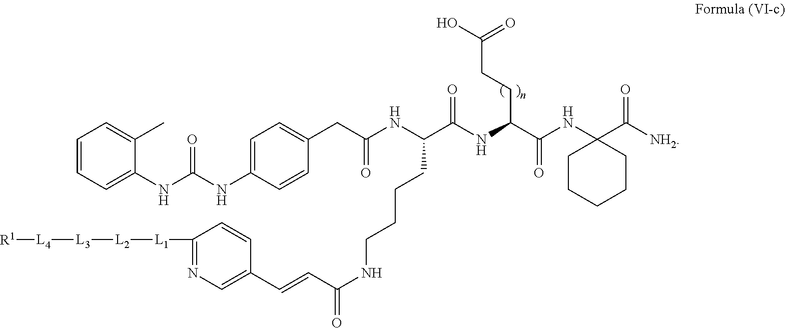

Embodiment 38. The compound, or a stereoisomer, tautomer, prodrug, or salt thereof, of embodiment 34, wherein the compound comprises the structure of Formula (VI-d):

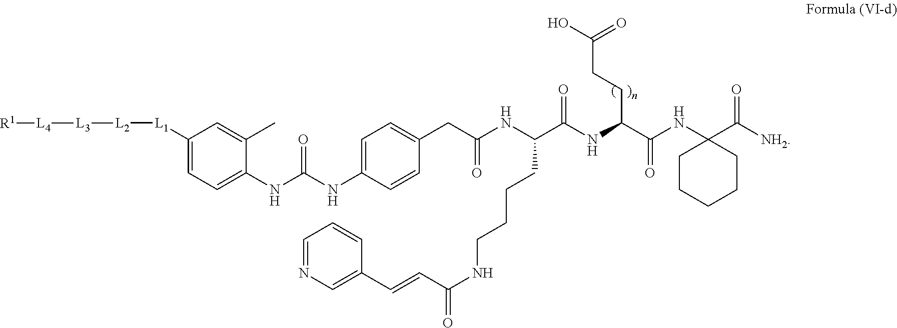

Embodiment 39. The compound, or a stereoisomer, tautomer, prodrug, or salt thereof, of embodiment 1, wherein the compound comprises the structure of Formula (VII′):

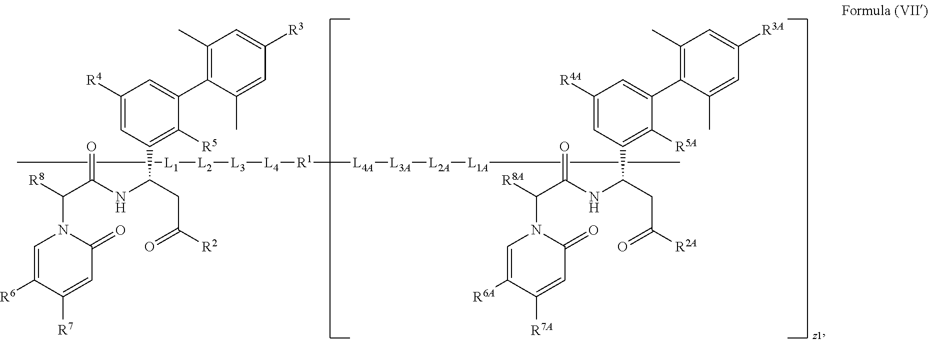

wherein
R², R^{2A}, R³, R^{3A}, R⁴ R^{4A} R⁵, and R^{5A} are each independently H, halogen, optionally substituted alkyl, optionally substituted —O-alkyl, cycloalkyl, or absent;
R⁸ and R^{8A} are each independently optionally substituted C₁-C₅ alkyl, optionally substituted C₁-C₅ alkylene-(C₃-C₆)-cycloalkyl, or optionally substituted (C₁-C₄)-alkylene-(C₁-C₄)-alkoxy;
R⁶, R^{6A}, R⁷, and R^{7A} are each independently H, halogen, alkyl, or optionally substituted alkyl, optionally substituted heteroalkyl,

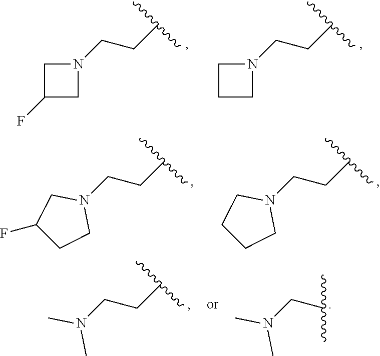

Embodiment 40. The compound, or a stereoisomer, tautomer, prodrug, or salt thereof, of embodiment 39, wherein the compound comprises the structure of Formula (VII′-a):

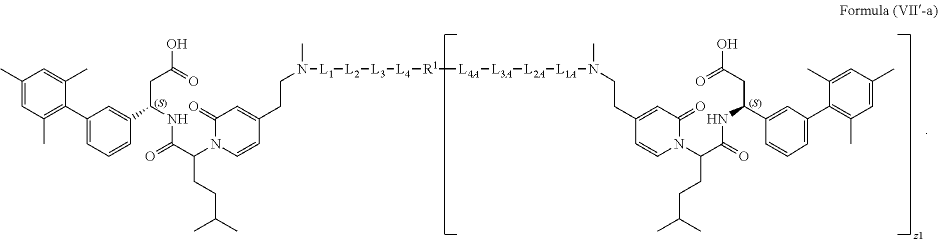

Embodiment 41. The compound, or a stereoisomer, tautomer, prodrug, or salt thereof, of embodiment 40, wherein the compound comprises the structure of Formula (VII′-a-1):

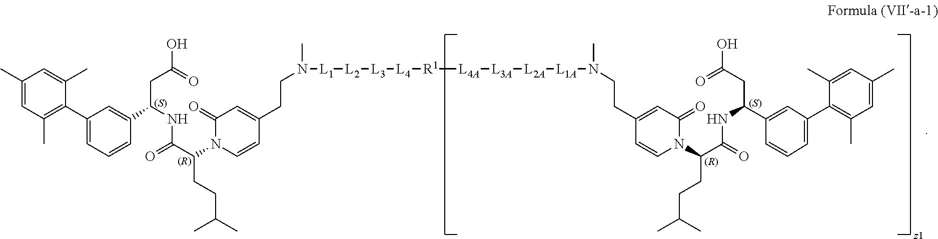

Embodiment 42. The compound, or a stereoisomer, tautomer, prodrug, or salt thereof, of embodiment 40, wherein the compound comprises the structure of Formula (VII′-a-2):

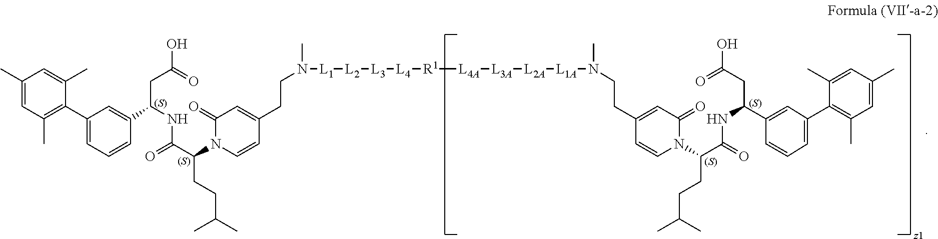

Embodiment 43. The compound, or a stereoisomer, tautomer, prodrug, or salt thereof, of embodiment 39, wherein the compound comprises the structure of Formula (VII):

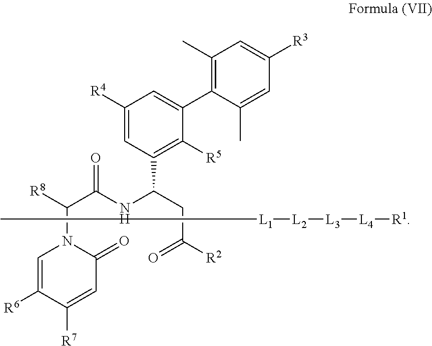

Embodiment 44. The compound, or a stereoisomer, tautomer, prodrug, or salt thereof, of embodiment 43, wherein the compound comprises the structure of Formula (VII-a):

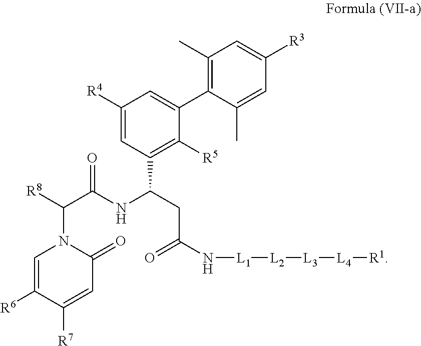

Embodiment 45. The compound, or a stereoisomer, tautomer, prodrug, or salt thereof, of embodiment 43, wherein the compound comprises the structure of Formula (VII-b):

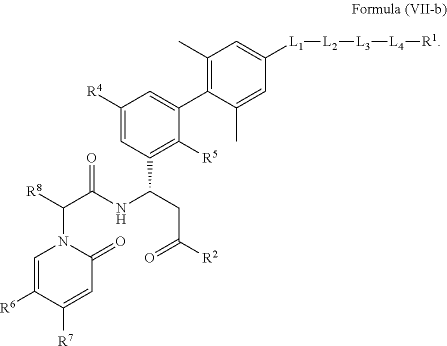

Embodiment 46. The compound, or a stereoisomer, tautomer, prodrug, or salt thereof, of embodiment 43, wherein the compound comprises the structure of Formula (VII-c):

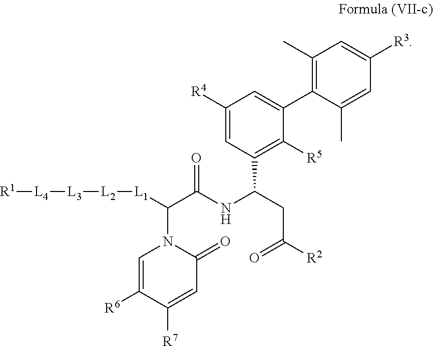

Embodiment 47. The compound, or a stereoisomer, tautomer, prodrug, or salt thereof, of embodiment 43, wherein the compound comprises the structure of Formula (VII-c-1):

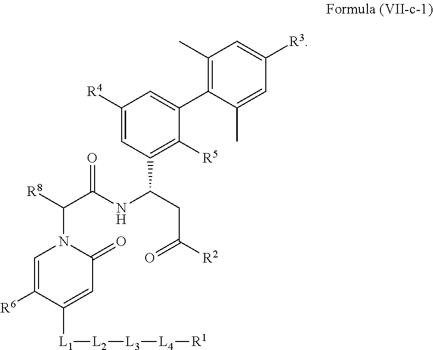

Embodiment 48. The compound, or a stereoisomer, tautomer, prodrug, or salt thereof, of embodiment 43, wherein the compound comprises the structure of Formula (VII-c-2):

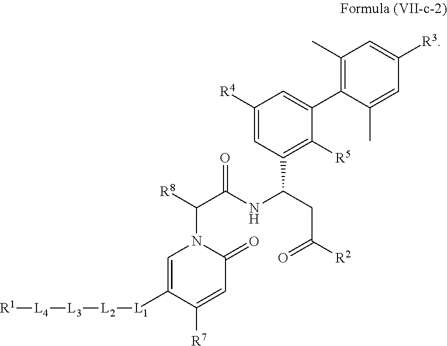

Embodiment 49. The compound, or a stereoisomer, tautomer, prodrug, or salt thereof, of embodiment 43, wherein the compound comprises the structure of Formula (VII-d):

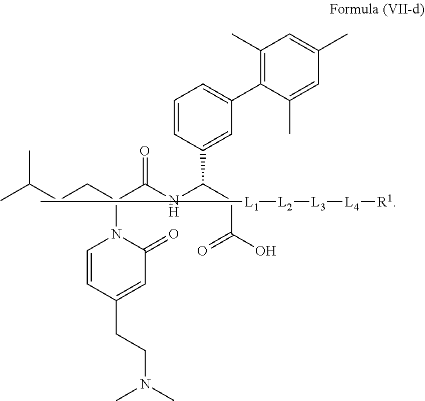

Embodiment 50. The compound, or a stereoisomer, tautomer, prodrug, or salt thereof, of embodiment 43, wherein the compound comprises the structure of Formula (VII-d-1):

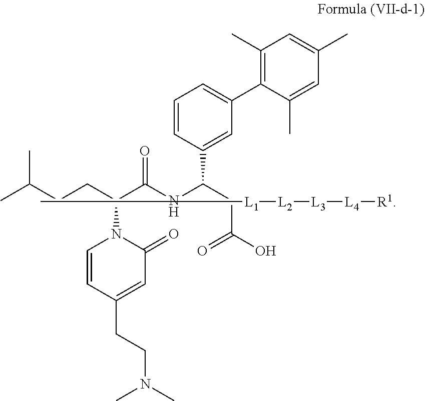

Embodiment 51. The compound, or a stereoisomer, tautomer, prodrug, or salt thereof, of embodiment 43, wherein the compound comprises the structure of Formula (VII-d-2):

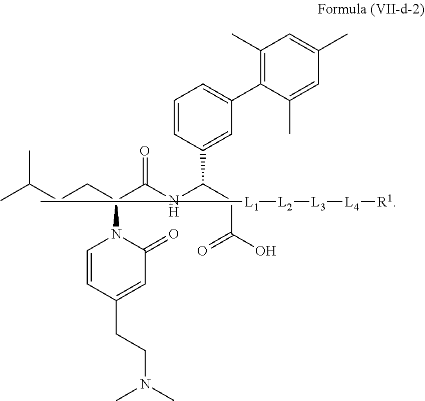

Embodiment 52. The compound, or a stereoisomer, tautomer, prodrug, or salt thereof, of embodiment 43, wherein the compound comprises the structure of Formula (VII-d-3):

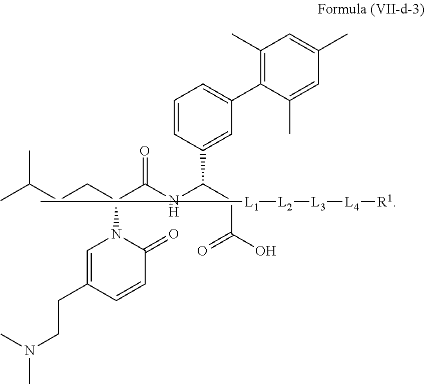

Embodiment 53. The compound, or a stereoisomer, tautomer, prodrug, or salt thereof, of embodiment 43, wherein the compound comprises the structure of Formula (VII-d-4):

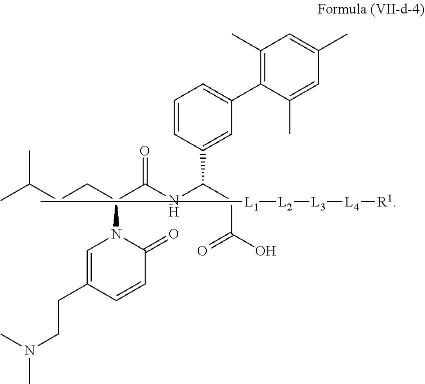

Embodiment 54. The compound, or a stereoisomer, tautomer, prodrug, or salt thereof, of embodiment 46, wherein the compound comprises the structure of Formula (VII-d-5):

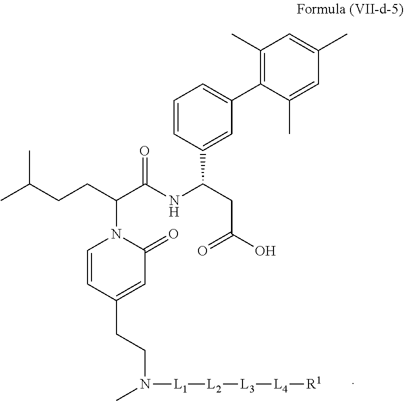

Embodiment 55. The compound, or a stereoisomer, tautomer, prodrug, or salt thereof, of embodiment 46, wherein the compound comprises the structure of Formula (VII-d-6):

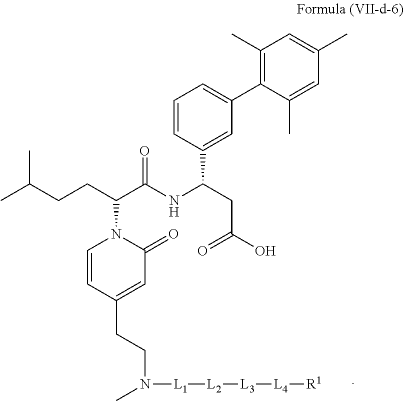

Embodiment 56. The compound, or a stereoisomer, tautomer, prodrug, or salt thereof, of embodiment 46, wherein the compound comprises the structure of Formula (VII-d-7):

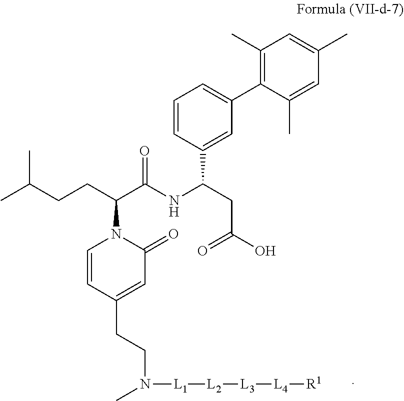

Embodiment 57. The compound, or a stereoisomer, tautomer, prodrug, or salt thereof, of embodiment 46, wherein the compound comprises the structure of Formula (VII-d-8):

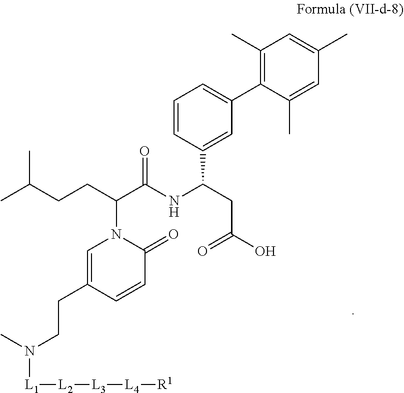

Embodiment 58. The compound, or a stereoisomer, tautomer, prodrug, or salt thereof, of embodiment 46, wherein the compound comprises the structure of Formula (VII-d-9):

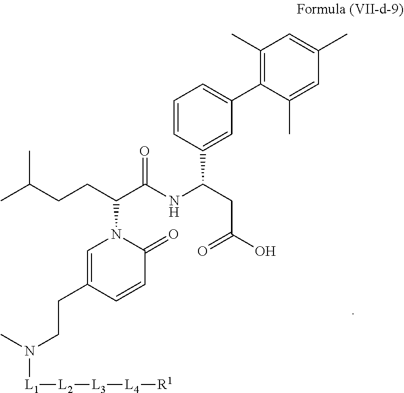

Embodiment 59. The compound, or a stereoisomer, tautomer, prodrug, or salt thereof, of embodiment 46, wherein the compound comprises the structure of Formula (VII-d-10):

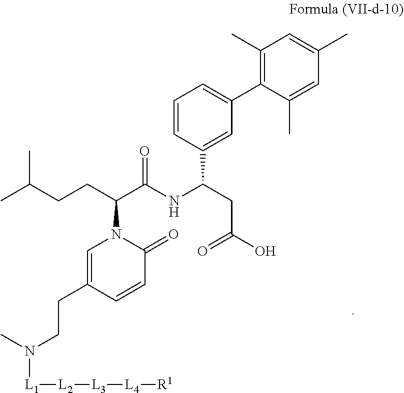

Embodiment 60. The compound of embodiment 39, wherein R⁶ is F, CF₃, or CH₃, and R⁷ is

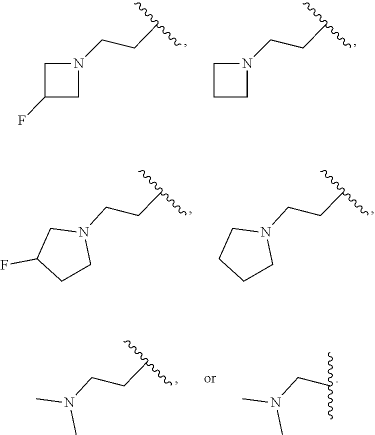

Embodiment 61. The compound of embodiment 39, wherein R⁷ is F, CF₃, or CH₃, and R⁶

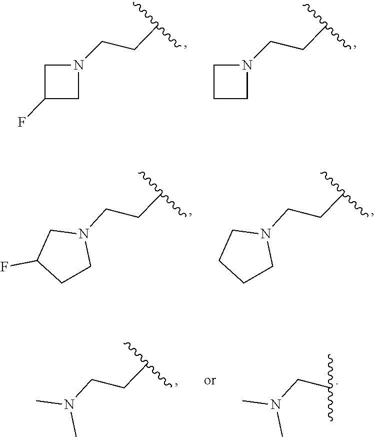

Embodiment 62. The compound, or a stereoisomer, tautomer, prodrug, or salt thereof, of embodiment 1, wherein the compound comprises the structure of Formula (VIII′):

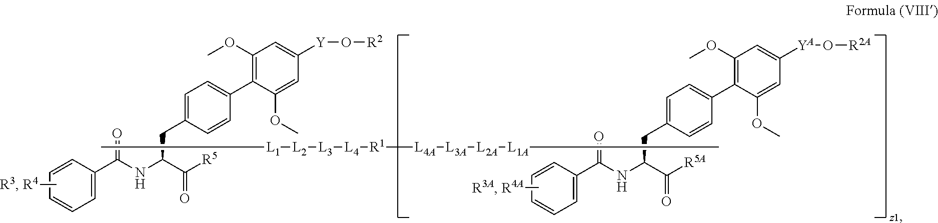

wherein
R² and R^{2A} are each independently H, polyethylene glycol, optionally substituted alkyl, optionally substituted heteroalkyl, optionally substituted cycloalkyl, optionally substituted heteroaryl, or absent;
R³, R^{3A}, R⁴, and R^{4A}, are each independently H, halogen, optionally substituted alkyl, or optionally substituted —O-alkyl;
R⁵ and R^{5A} are each independently —OH or absent; Y and Y^{A} are each independently —CH₂— or —(CH₂)₂—.

Embodiment 63. The compound, or a stereoisomer, tautomer, prodrug, or salt thereof, of embodiment 62, wherein the compound comprises the structure of Formula (VIII′-a):

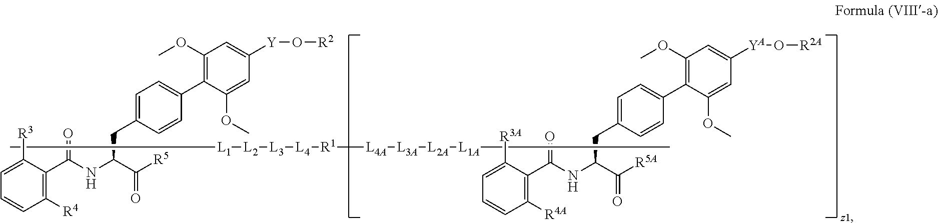

Embodiment 64. The compound, or a stereoisomer, tautomer, prodrug, or salt thereof, of embodiment 62, wherein the compound comprises the structure of Formula (VIII):

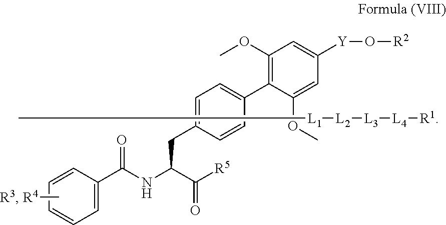

Embodiment 65. The compound, or a stereoisomer, tautomer, prodrug, or salt thereof, of embodiment 64, wherein the compound comprises the structure of Formula (VIII-a):

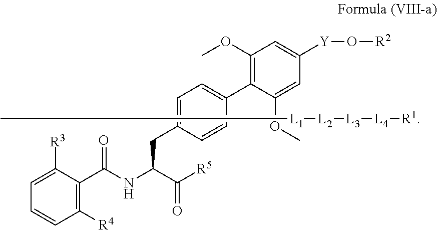

Embodiment 66. The compound, or a stereoisomer, tautomer, prodrug, or salt thereof, of embodiment 65, wherein the compound comprises the structure of Formula (VIII-a-1):

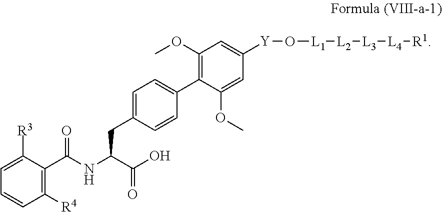

Embodiment 67. The compound, or a stereoisomer, tautomer, prodrug, or salt thereof, of embodiment 65, wherein the compound comprises the structure of Formula (VIII-a-2):

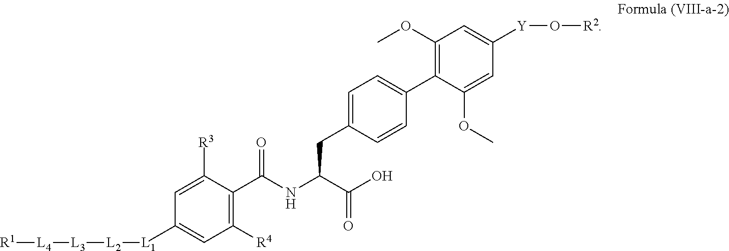

Embodiment 68. The compound, or a stereoisomer, tautomer, prodrug, or salt thereof, of embodiment 65, wherein the compound comprises the structure of Formula (VIII-a-3):

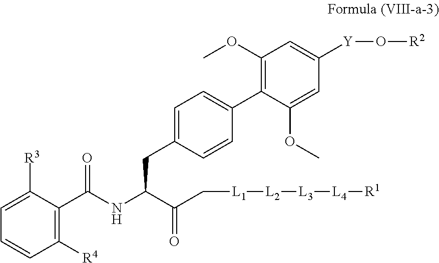

Embodiment 69. The compound, or a stereoisomer, tautomer, prodrug, or salt thereof, of embodiment 1, wherein the compound comprises the structure of Formula (IX′):

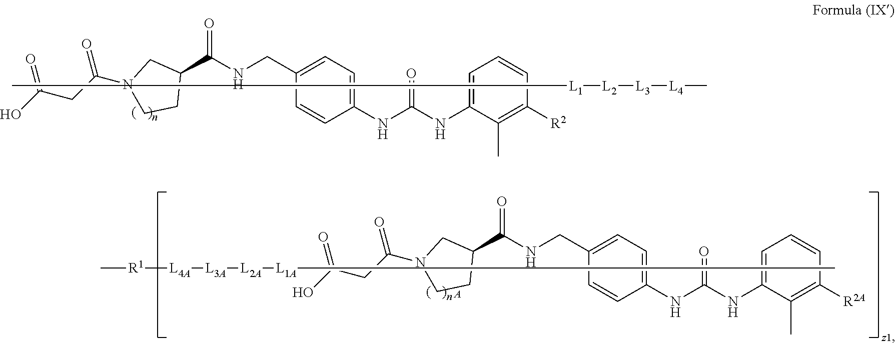

wherein
each of R² and R^{2A} is independently H, —OH, —NH₂, —NHR³, —OR³, or —CONHR³;
each instance of R³ is independently H, polyethylene glycol, optionally substituted alkyl, optionally substituted heteroalkyl, optionally substituted cycloalkyl, or optionally substituted heteroaryl; and
each of n and n^{A} is independently 1 or 2.

Embodiment 70. The compound, or a stereoisomer, tautomer, prodrug, or salt thereof, of embodiment 69, wherein the compound comprises the structure of Formula (IX):

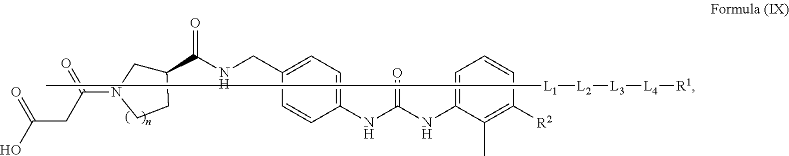

Embodiment 71. The compound, or a stereoisomer, tautomer, prodrug, or salt thereof, of embodiment 70, wherein the compound comprises the structure of Formula (IX-a):

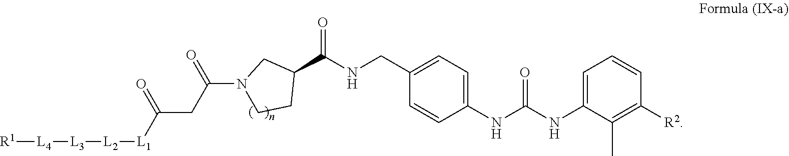

Embodiment 72. The compound, or a stereoisomer, tautomer, prodrug, or salt thereof, of embodiment 70, wherein the compound comprises the structure of Formula (IX-b):

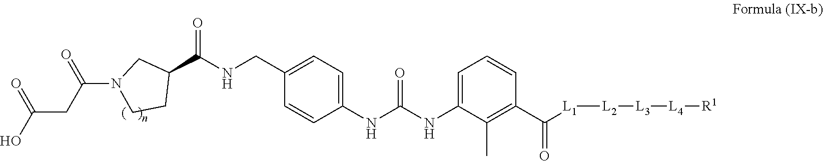

Embodiment 73. The compound, or a stereoisomer, tautomer, prodrug, or salt thereof, of embodiment 1, wherein the compound comprises the structure of Formula (X′):

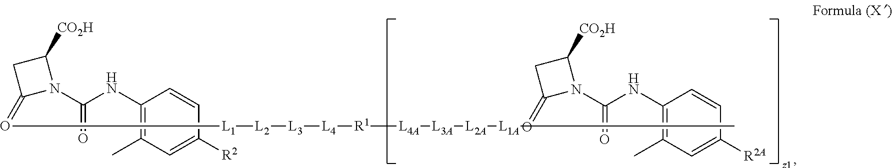

wherein
R² and R^{2A} are each independently H, —CH₂OR³, —(CH₂)₂OR³, —CH₂NHCOR³, or —OR³; and
each instance of R³ is independently H, polyethylene glycol, optionally substituted alkyl, optionally substituted heteroalkyl, optionally substituted cycloalkyl, or optionally substituted heteroaryl.

Embodiment 74. The compound, or a stereoisomer, tautomer, prodrug, or salt thereof, of embodiment 73, wherein the compound comprises the structure of Formula (X):

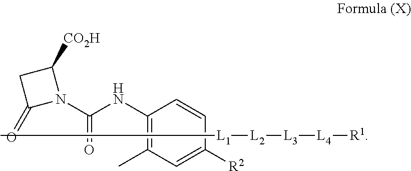

Embodiment 75. The compound, or a stereoisomer, tautomer, prodrug, or salt thereof, of embodiment 74, wherein the compound comprises the structure of Formula (X-a):

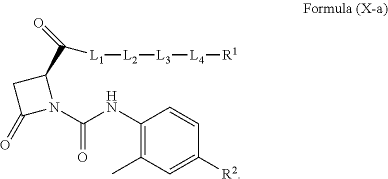

Embodiment 76. The compound, or a stereoisomer, tautomer, prodrug, or salt thereof, of embodiment 74, wherein the compound comprises the structure of Formula (X-b):

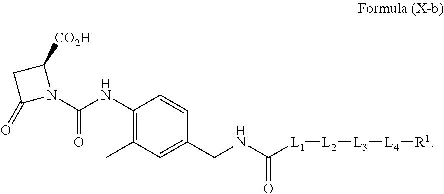

Embodiment 77. The compound, or a stereoisomer, tautomer, prodrug, or salt thereof, of embodiment 1, wherein the compound comprises the structure of Formula (XI′):

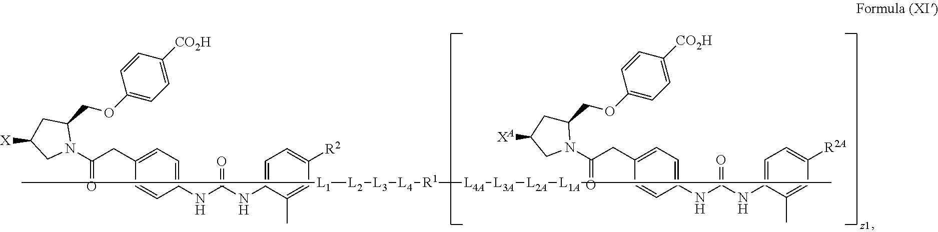

wherein
each of R² and R^{2A} is independently H, —CONHR³, —CH₂OR³, —(CH₂)₂OR³, —CH₂NHCOR³, or —OR³;
each instance of R³ is independently H, polyethylene glycol, optionally substituted alkyl, optionally substituted heteroalkyl, optionally substituted cycloalkyl, or optionally substituted heteroaryl; and
each of X and X^{A} are independently H or halogen.

Embodiment 78. The compound, or a stereoisomer, tautomer, prodrug, or salt thereof, of embodiment 77, wherein the compound comprises the structure of Formula (XI):

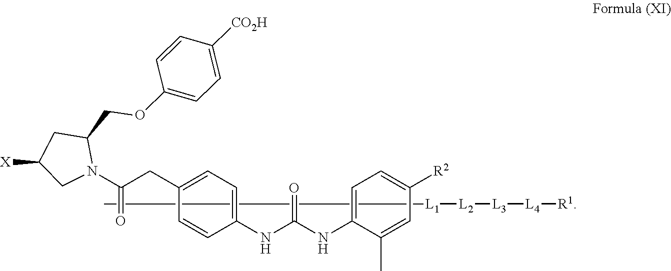

Embodiment 79. The compound, or a stereoisomer, tautomer, prodrug, or salt thereof, of embodiment 78, wherein the compound comprises the structure of Formula (XI-a):

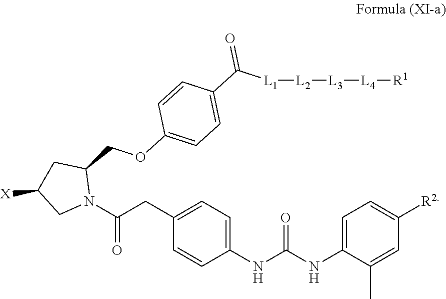

Embodiment 80. The compound, or a stereoisomer, tautomer, prodrug, or salt thereof, of embodiment 78, wherein the compound comprises the structure of Formula (XI-b):

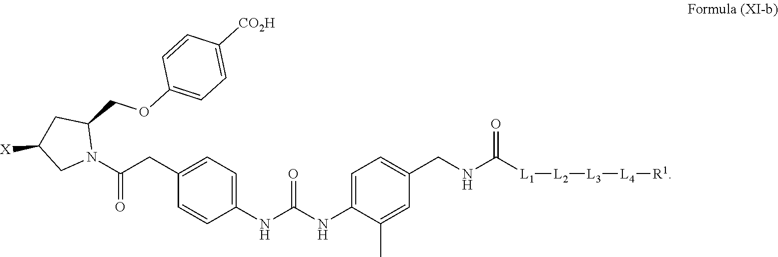

Embodiment 81. The compound, or a stereoisomer, tautomer, prodrug, or salt thereof, of embodiment 1, wherein the compound comprises the structure of Formula (XII′):

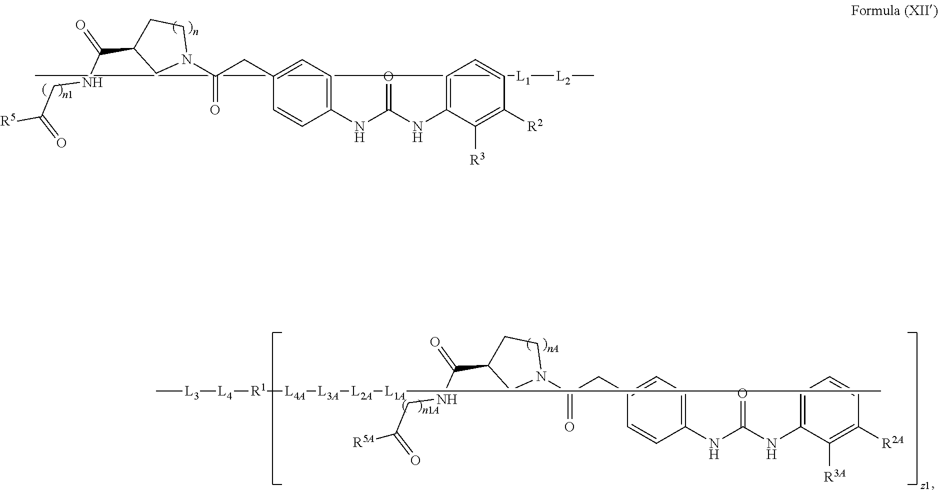

wherein
each of R² and R^{2A} is independently H, —CONHR⁴, —CH₂OR⁴, —(CH₂)₂OR⁴, —CH₂NHCOR⁴, or —OR⁴;
each of R³ and R^{3A} is independently H, optionally substituted alkyl, or optionally substituted cycloalkyl;
each instance of R⁴ is independently H, polyethylene glycol, optionally substituted alkyl, optionally substituted heteroalkyl, optionally substituted cycloalkyl, or optionally substituted heteroaryl;
each of R⁵ and R^{5A} is independently —OH or absent;
each instance of n and n^{A} is independently 0, 1, 2, or 3; and
each instance of n1 and n1^{A} is independently 1, 2, or 3.

Embodiment 82. The compound, or a stereoisomer, tautomer, prodrug, or salt thereof, of embodiment 81, wherein the compound comprises the structure of Formula (XII):

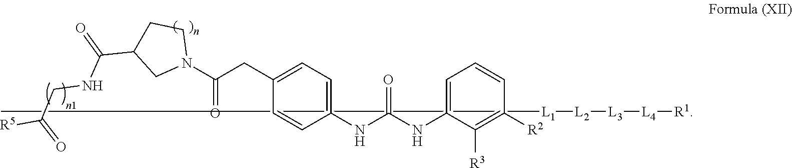

Embodiment 83. The compound, or a stereoisomer, tautomer, prodrug, or salt thereof, of embodiment 82, wherein the compound comprises the structure of Formula (XII-a):

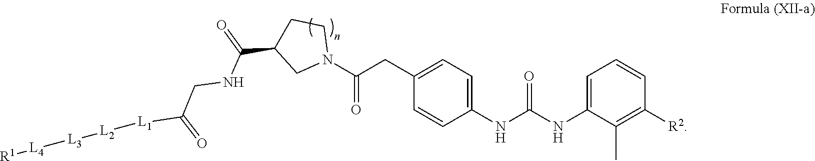

Embodiment 84. The compound, or a stereoisomer, tautomer, prodrug, or salt thereof, of embodiment 82, wherein the compound comprises the structure of Formula (XII-b):

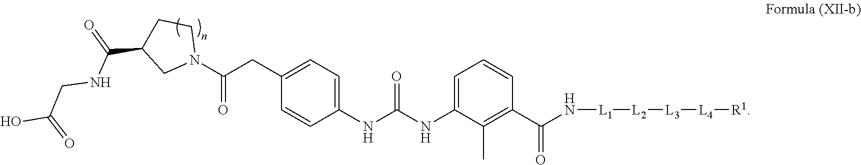

Embodiment 85. The compound, or a stereoisomer, tautomer, prodrug, or salt thereof, of embodiment 1, wherein the compound comprises the structure of Formula (XIII′):

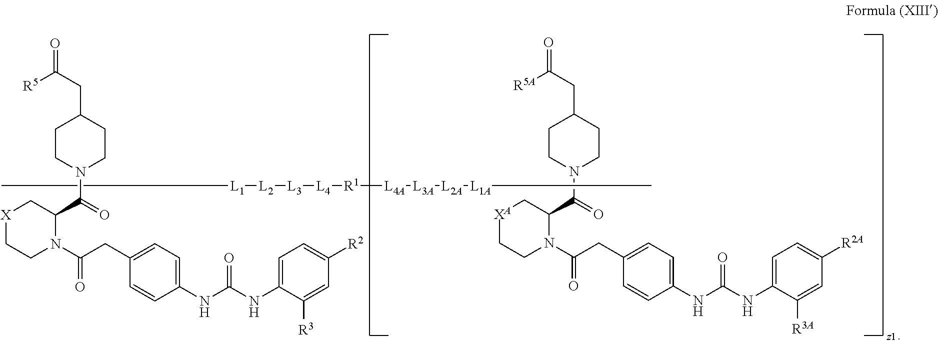

wherein
each of R² and R^{2A} is independently H, —CONHR⁴, —CH₂OR⁴, —(CH₂)₂OR⁴, —CH₂NHCOR⁴, or —OR⁴;
each of R³ and R^{3A} is independently H, optionally substituted alkyl, or optionally substituted cycloalkyl;
each instance of R⁴ is independently H, polyethylene glycol, optionally substituted alkyl, optionally substituted heteroalkyl, optionally substituted cycloalkyl, or optionally substituted heteroaryl;
each of R⁵ and R^{5A} is independently —OH or absent; and each of X and X^{A} is independently H, optionally substituted CH₂, optionally substituted NH, or cycloalkyl.

Embodiment 86. The compound, or a stereoisomer, tautomer, prodrug, or salt thereof, of embodiment 85, wherein the compound comprises the structure of Formula (XIII):

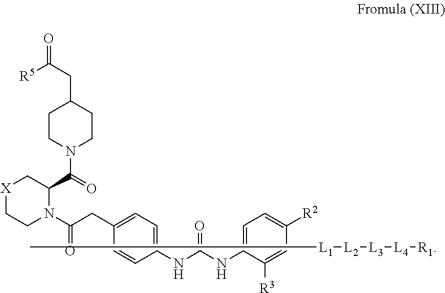

Embodiment 87. The compound, or a stereoisomer, tautomer, prodrug, or salt thereof, of embodiment 86, wherein the compound comprises the structure of Formula (XIII-a):

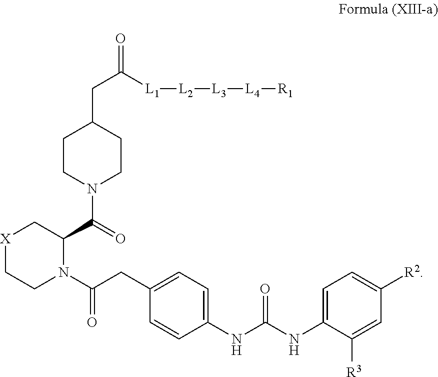

Embodiment 88. The compound, or a stereoisomer, tautomer, prodrug, or salt thereof, of embodiment 86, wherein the compound comprises the structure of Formula (XIII-b):

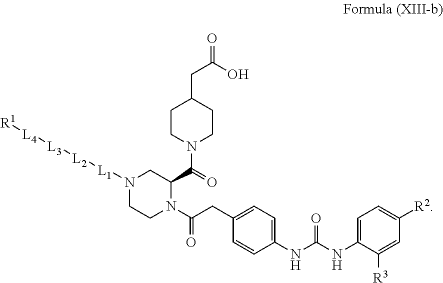

Embodiment 89. The compound, or a stereoisomer, tautomer, prodrug, or salt thereof, of embodiment 86, wherein the compound comprises the structure of Formula (XIII-c):

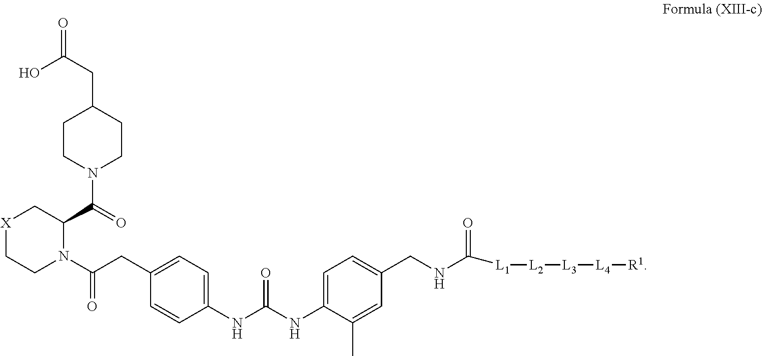

Embodiment 90. The compound, or a stereoisomer, tautomer, prodrug, or salt thereof, of embodiment 1, wherein the compound comprises the structure of Formula (XIV′):

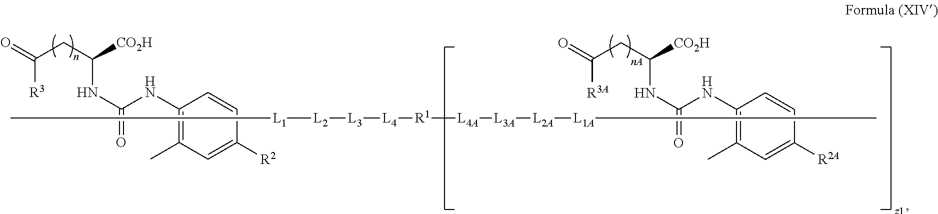

wherein
each of R² and R^{2A} is independently H, —CH₂OR⁴, —(CH₂)₂OR⁴, —CH₂NHCOR⁴, or —OR⁴;
each of R³ and R^{3A} is independently H, —OH, —NH₂, —NHR⁵, or —OR⁵;
each instance of R⁴ is independently H, polyethylene glycol, optionally substituted alkyl, optionally substituted heteroalkyl, optionally substituted cycloalkyl, or optionally substituted heteroaryl;
each instance of R⁵ is independently H, polyethylene glycol, optionally substituted alkyl, optionally substituted heteroalkyl, optionally substituted cycloalkyl, or optionally substituted heteroaryl; and
each of n and n^{A} is independently 1, 2, or 3.

Embodiment 91. The compound, or a stereoisomer, tautomer, prodrug, or salt thereof, of embodiment 90, wherein the compound comprises the structure of Formula (XIV):

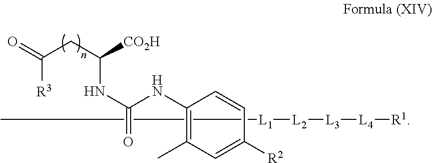

Embodiment 92. The compound, or a stereoisomer, tautomer, prodrug, or salt thereof, of embodiment 91, wherein the compound comprises the structure of Formula (XIV-a):

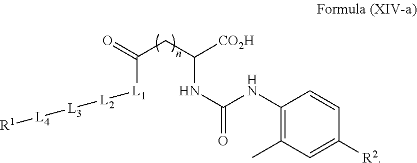

Embodiment 93. The compound, or a stereoisomer, tautomer, prodrug, or salt thereof, of embodiment 91, wherein the compound comprises the structure of Formula (XIV-b):

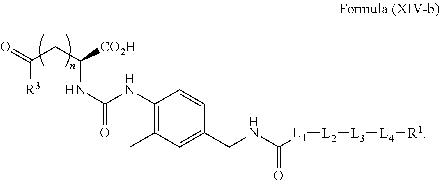

Embodiment 94. The compound, or a stereoisomer, tautomer, prodrug, or salt thereof, of any one of embodiments 1-93, wherein each of L₁, L₂, L₃, L₄, L_{1A}, L_{2A}, L_{3A}, and L_{4A} is independently absent, a bond, an optionally substituted alkyl linker, an optionally substituted polyethylene glycol (PEG) linker, an optionally substituted heteroalkyl linker, an optionally substituted heteroaryl linker, an optionally substituted saturated or partially unsaturated heterocycloalkyl linker, oxygen, optionally substituted nitrogen, an amide, a phosphodiester bond, or a phosphorothioate bond.

Embodiment 95. The compound, or a stereoisomer, tautomer, prodrug, or salt thereof, of embodiment 94, wherein L₁ and/or L_{1A} is a bond.

Embodiment 96. The compound, or a stereoisomer, tautomer, prodrug, or salt thereof, of embodiment 94 or 95, wherein L₂ and/or L_{2A} is an optionally substituted PEG linker.

Embodiment 97. The compound, or a stereoisomer, tautomer, prodrug, or salt thereof, of embodiment 96, wherein the PEG linker is two, three, four, five, six, seven, eight, nine, or ten PEG units in length.

Embodiment 98. The compound, or a stereoisomer, tautomer, prodrug, or salt thereof, of any one of embodiments 94-97, wherein L₂ and/or L_{2A} comprises the structure

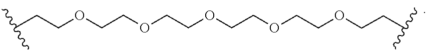 [SMILES: *CCOCCOCCOCCOCCOCC*]

Embodiment 99. The compound, or a stereoisomer, tautomer, prodrug, or salt thereof, of any one of embodiments 94-98, wherein L₃ and/or L_{3A} is an optionally substituted heteroaryl linker.

Embodiment 100. The compound, or a stereoisomer, tautomer, prodrug, or salt thereof, of embodiment 99, wherein L₃ and/or L_{3A} is an optionally substituted partially unsaturated heteroaryl linker.

Embodiment 101. The compound, or a stereoisomer, tautomer, prodrug, or salt thereof, of

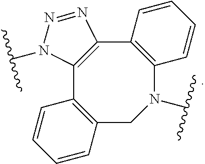 [SMILES: *N1Cc2ccccc2-c2c(nnn2*)-c2ccccc21]

embodiment 99 or 100, wherein L₃ and/or L_{3A} comprises the structure

Embodiment 102. The compound, or a stereoisomer, tautomer, prodrug, or salt thereof, of any one of embodiments 94-101, wherein L₄ and/or L_{4A} is an optionally substituted heteroalkyl linker.

Embodiment 103. The compound, or a stereoisomer, tautomer, prodrug, or salt thereof, of embodiment 102, wherein the heteroalkyl linker is substituted with one or more ═O substituents.

Embodiment 104. The compound, or a stereoisomer, tautomer, prodrug, or salt thereof, of embodiment 102 or 103, wherein the heteroalkyl linker comprises two substituents joined together to form an optionally substituted carbocyclyl ring.

Embodiment 105. The compound, or a stereoisomer, tautomer, prodrug, or salt thereof, of embodiment 102 or 103, wherein L₄ and/or L_{4A} comprises the structure

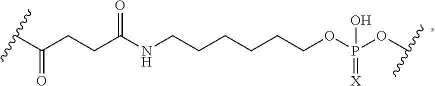

wherein X is O or S.

Embodiment 106. The compound, or a stereoisomer, tautomer, prodrug, or salt thereof, of any one of embodiments 102-104, wherein L₄ and/or L_{4A} comprises the structure

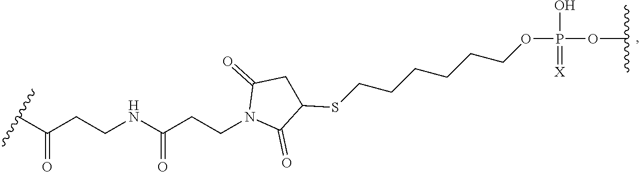

wherein X is O or S.

Embodiment 107. The compound, or a stereoisomer, tautomer, prodrug, or salt thereof, of any one of embodiments 94-106, wherein L₁, L₂, L₃, and L₄ and/or L_{1A}, L_{2A}, L_{3A}, and L_{4A} together comprise the structure

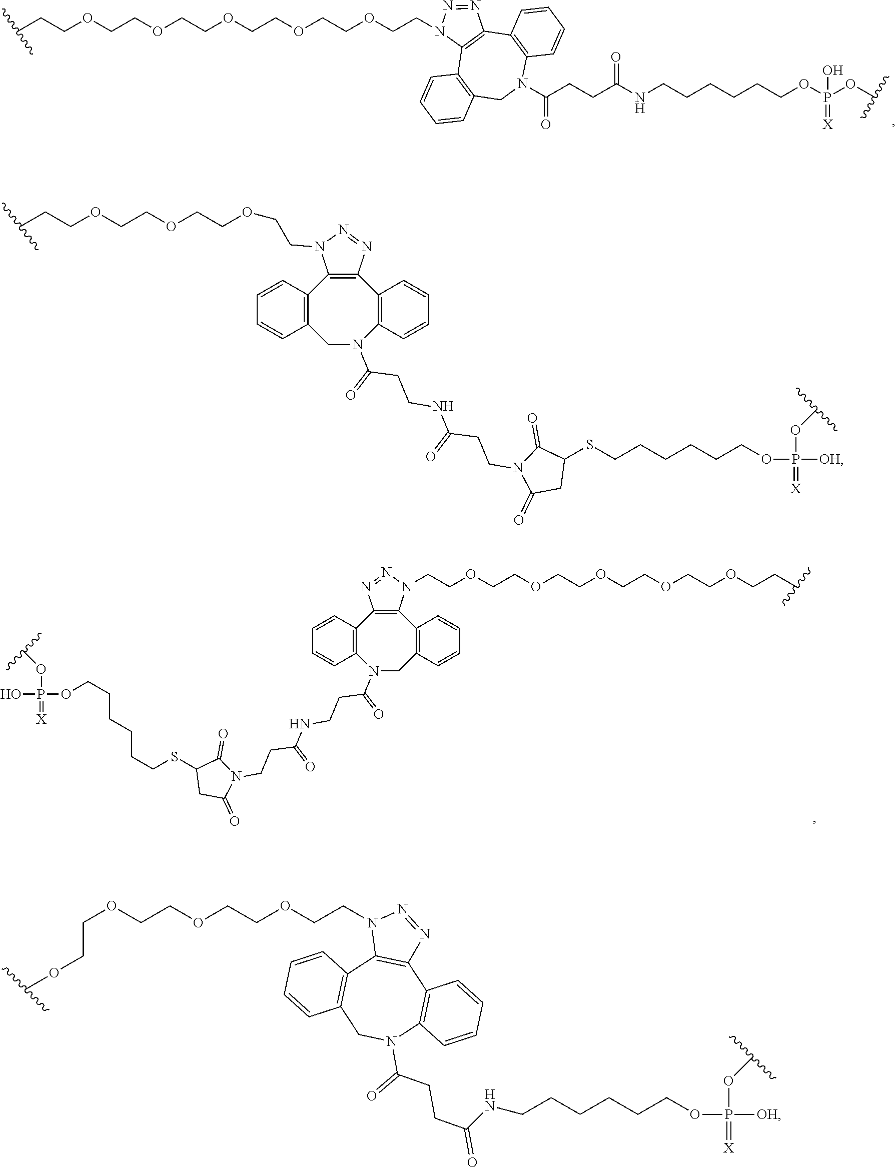

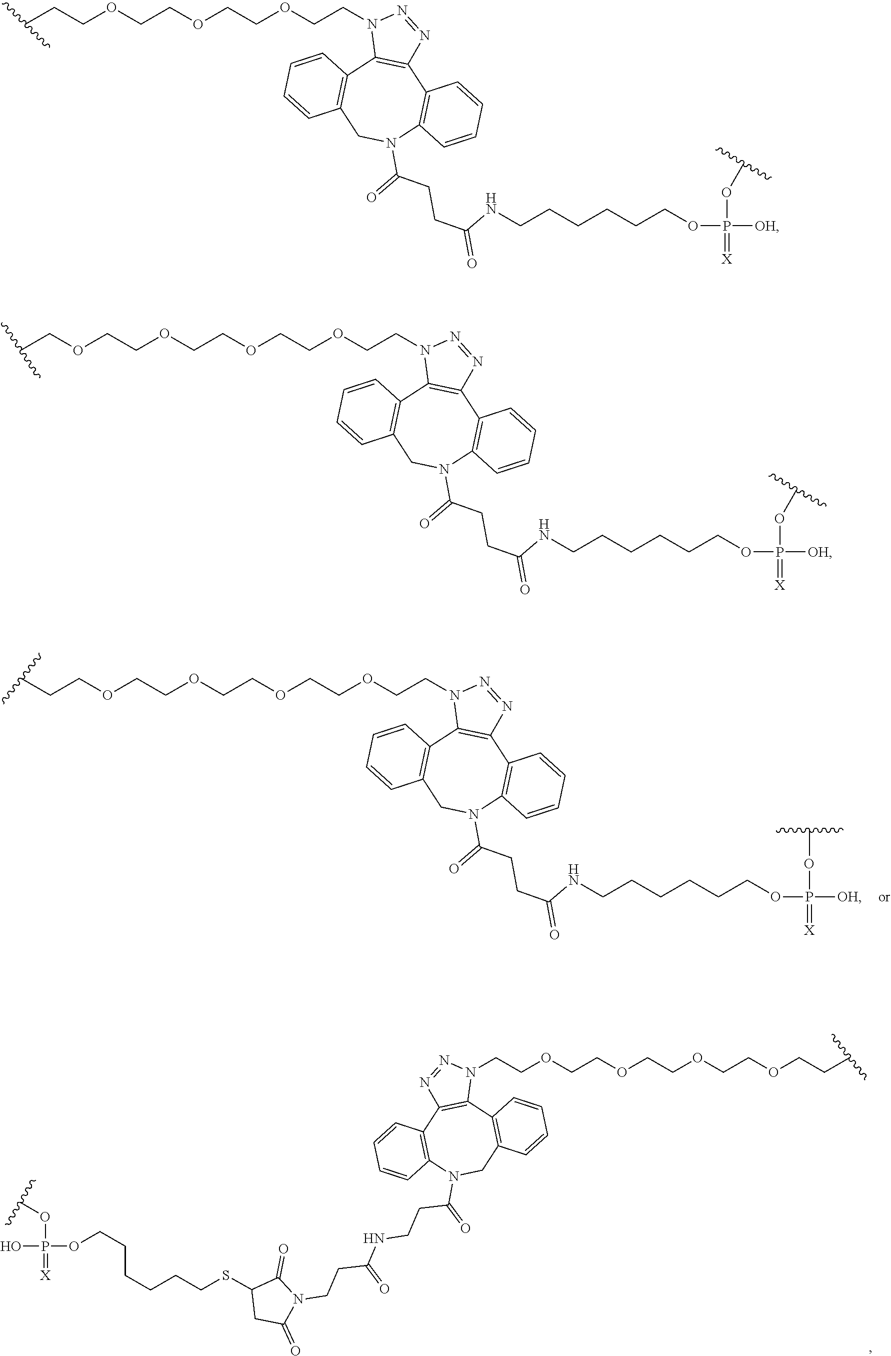

wherein X is O or S.

Embodiment 108. The compound, or a stereoisomer, tautomer, prodrug, or salt thereof, of any one of embodiments 1-107, wherein the compound comprises the structure:

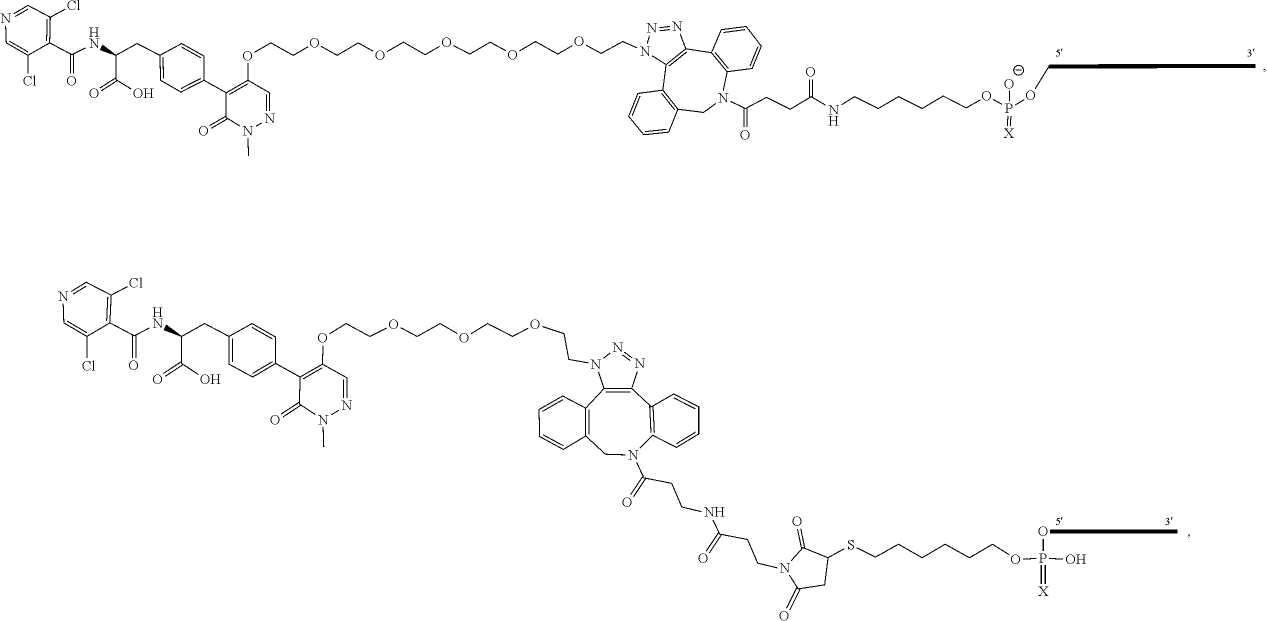

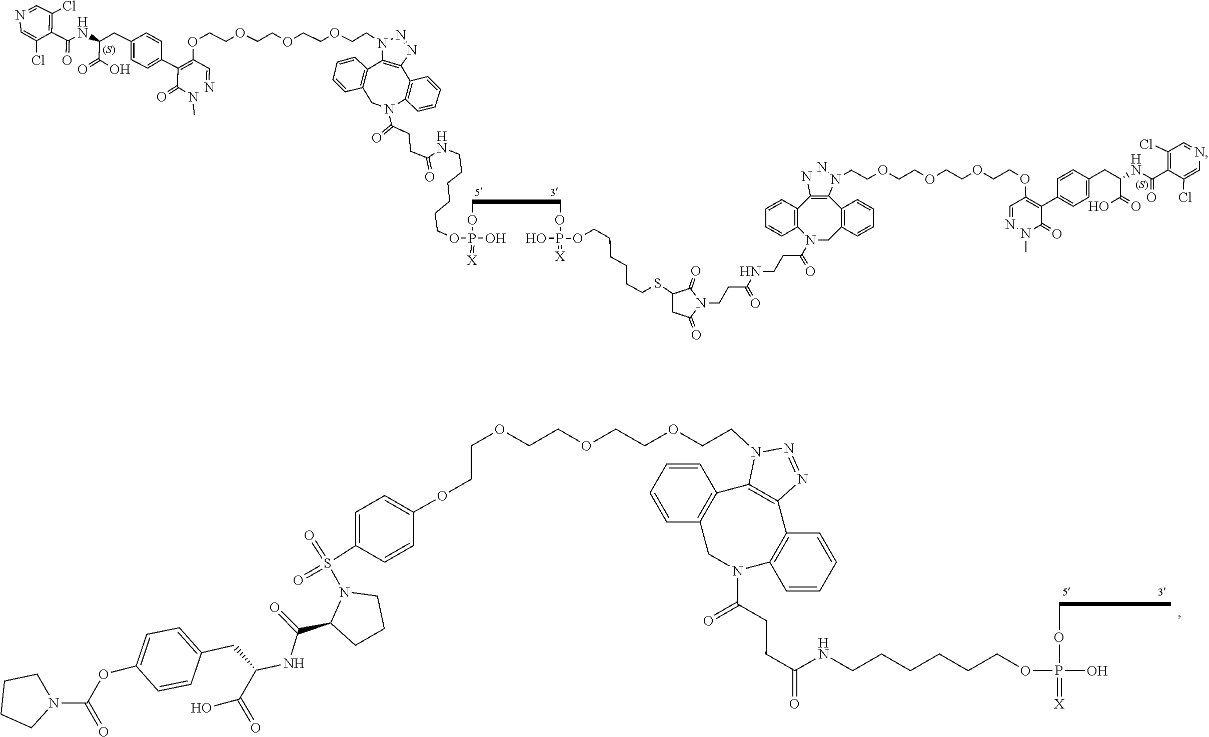

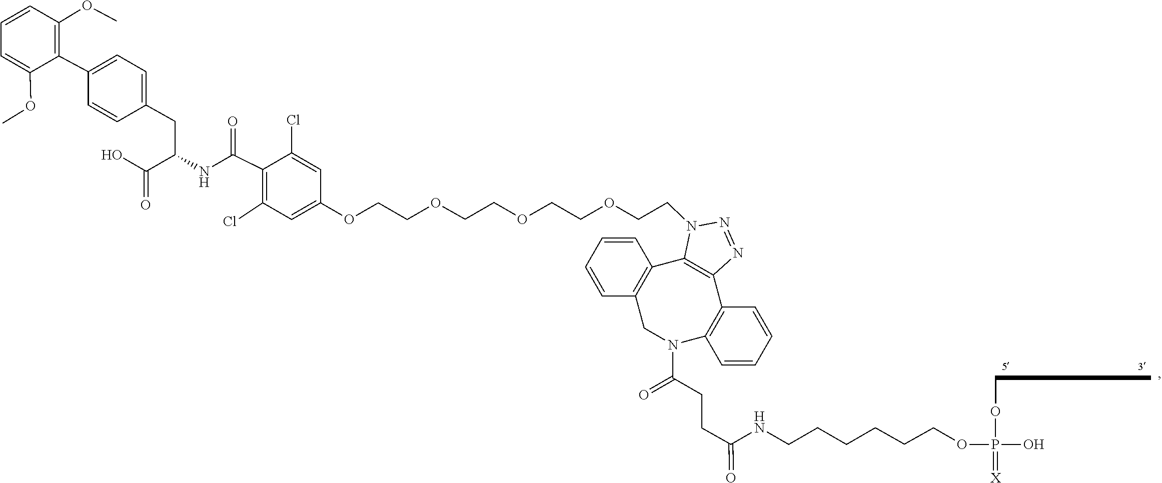

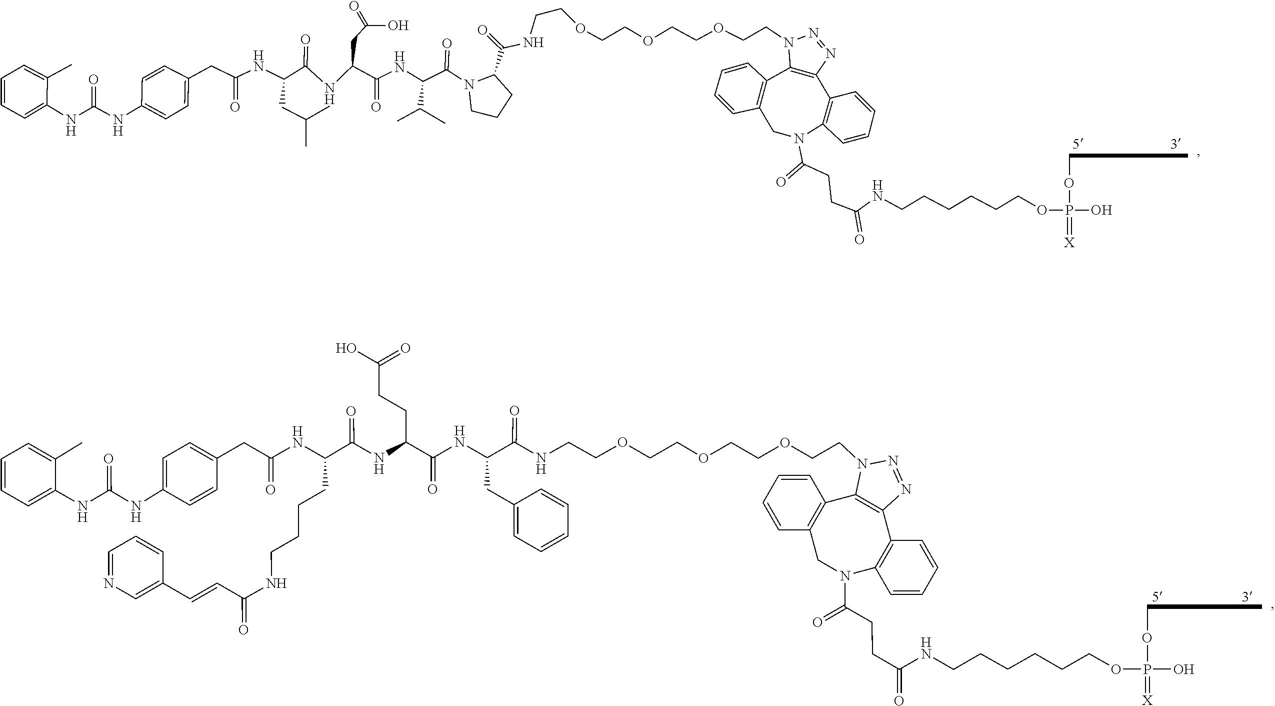

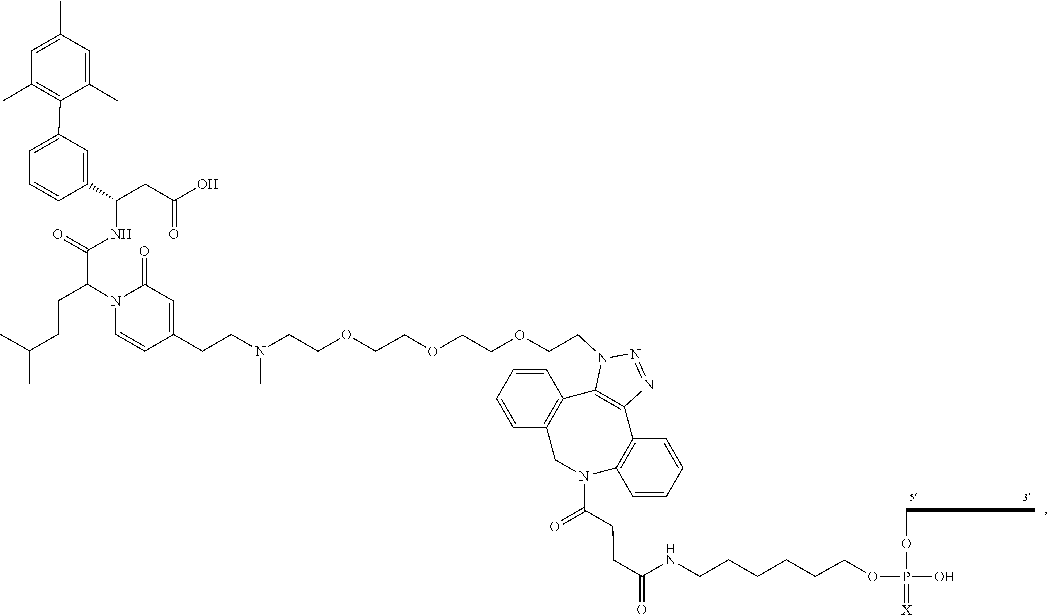

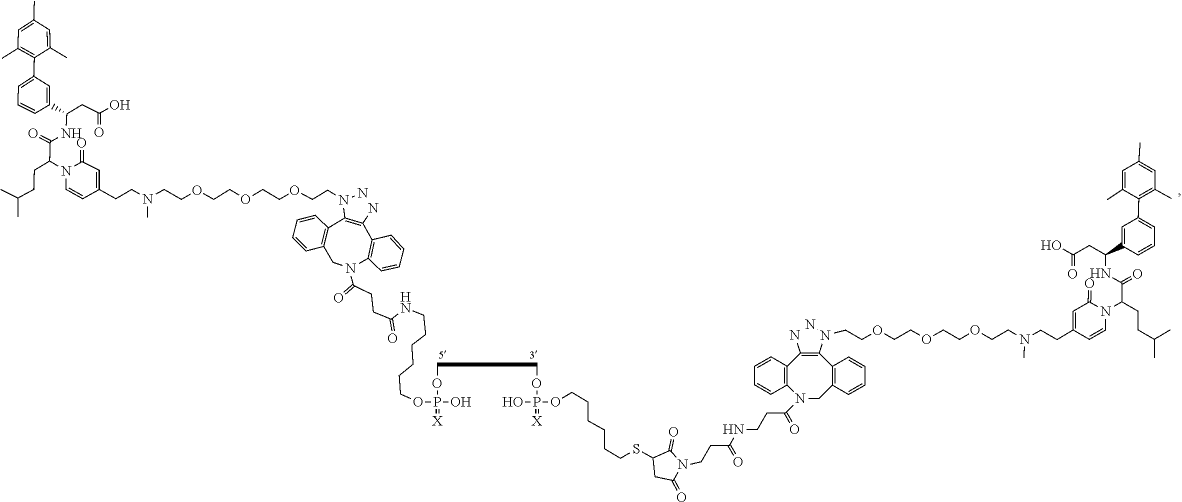

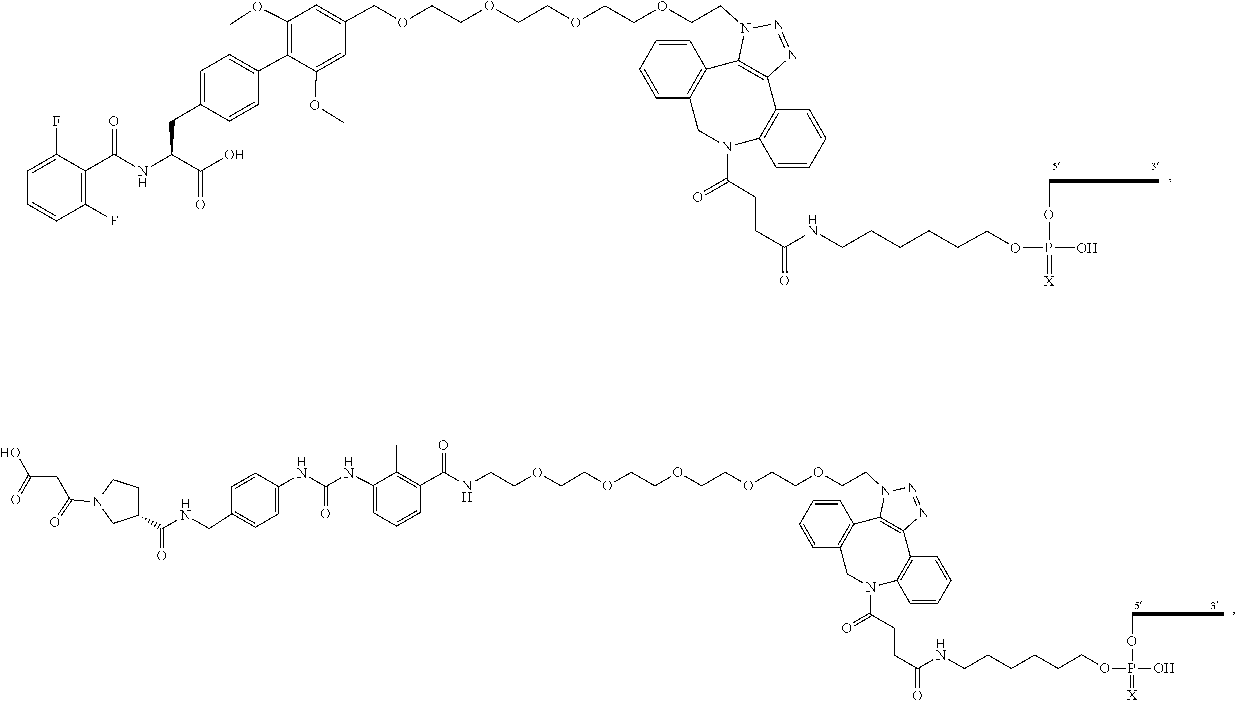

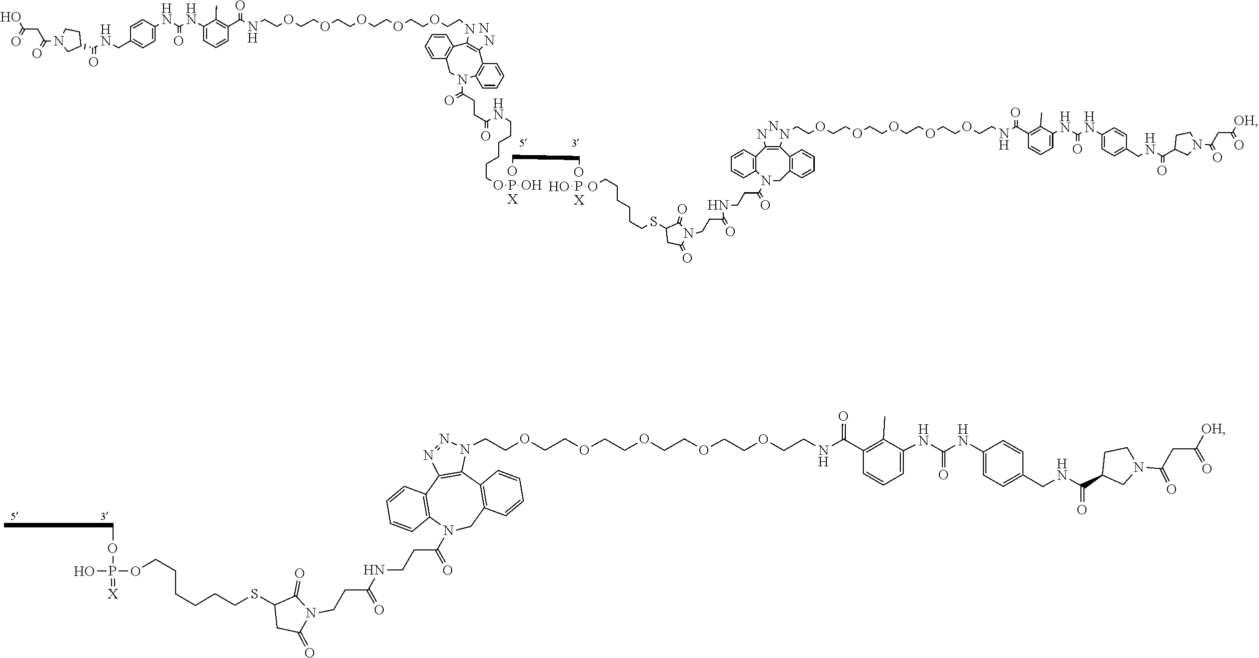

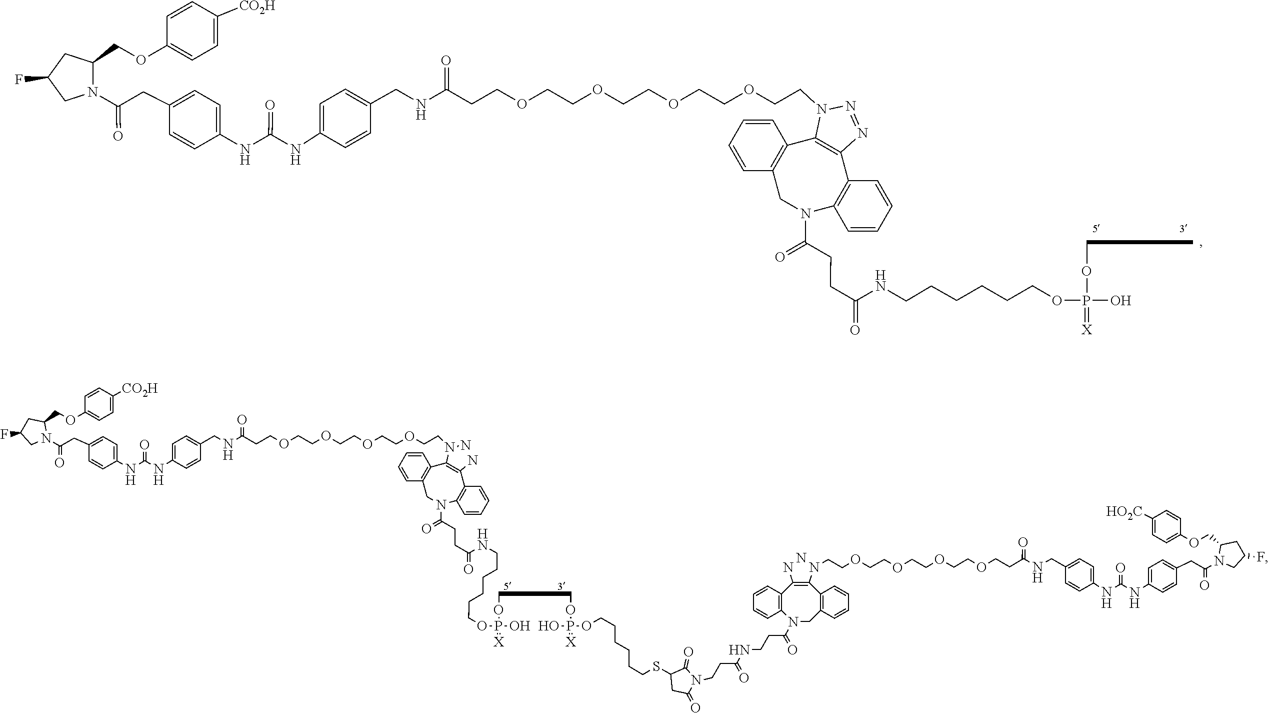

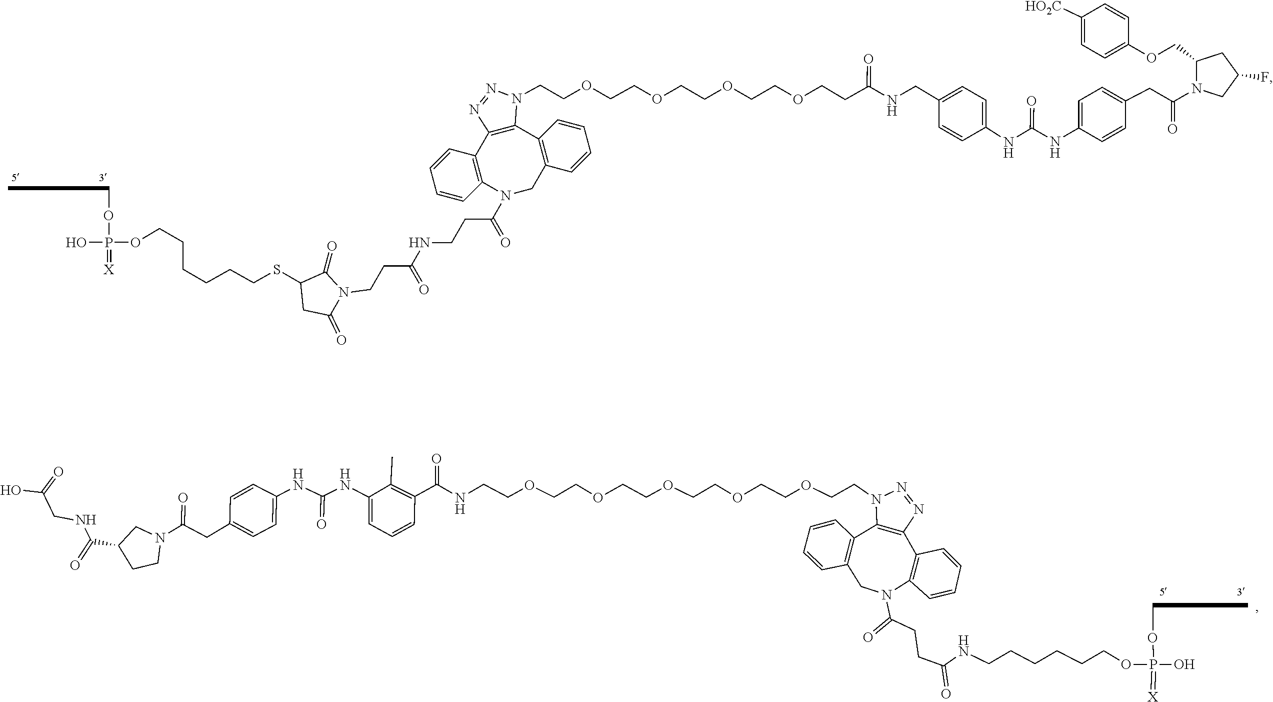

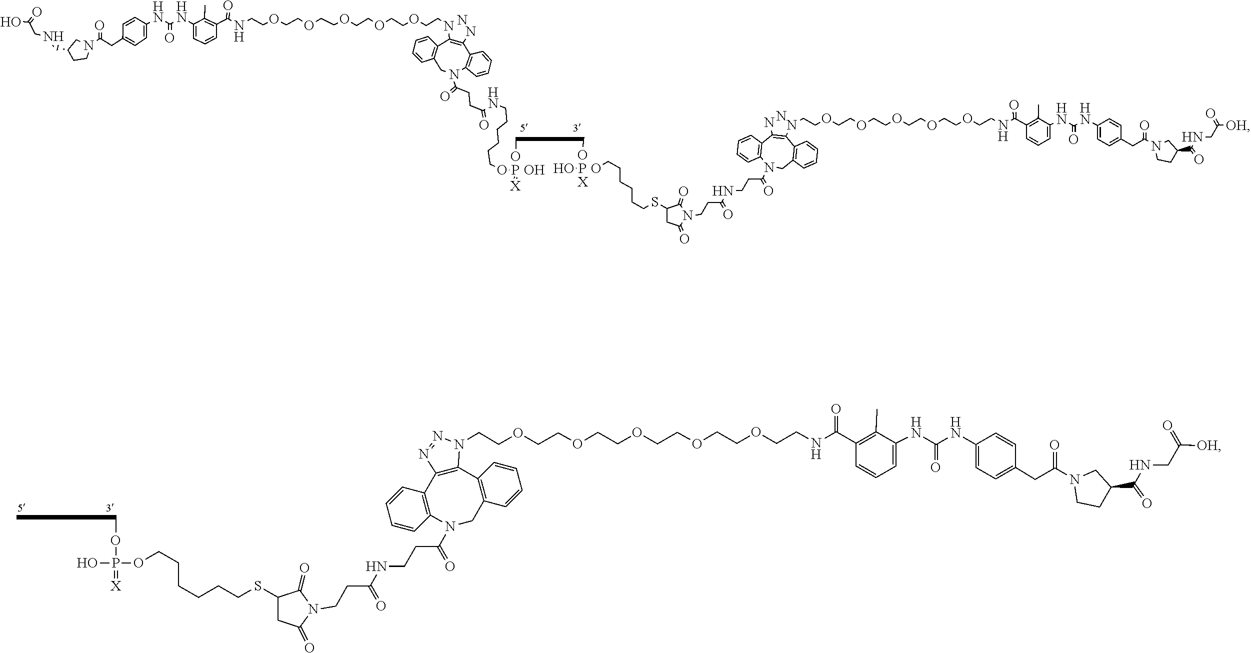

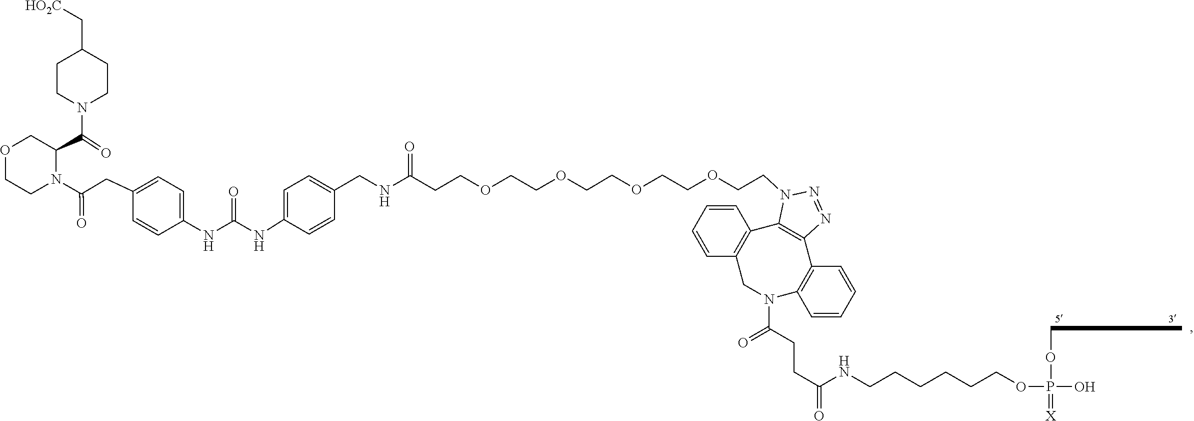

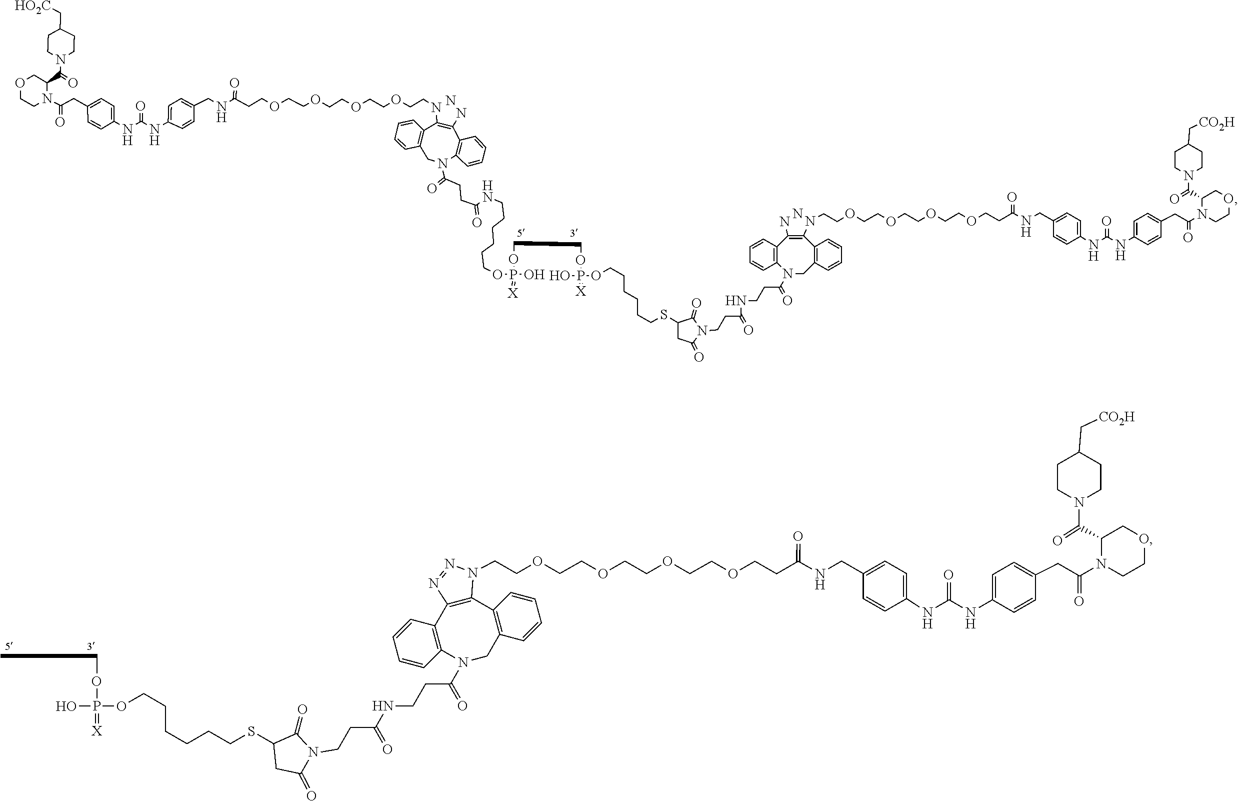

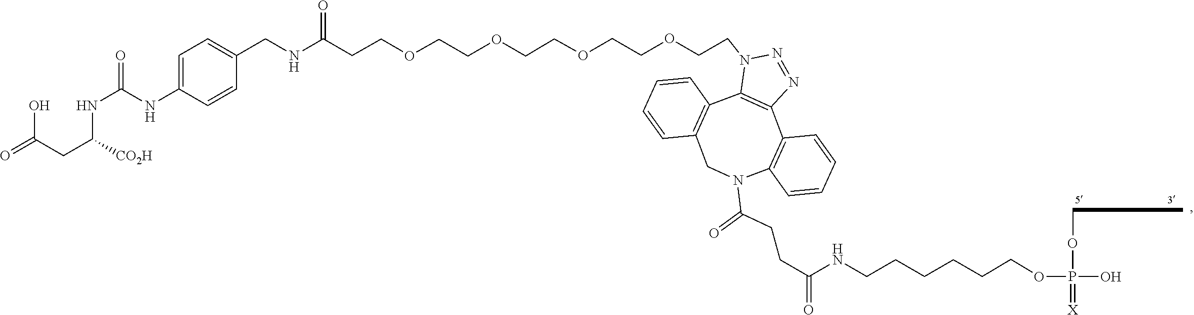

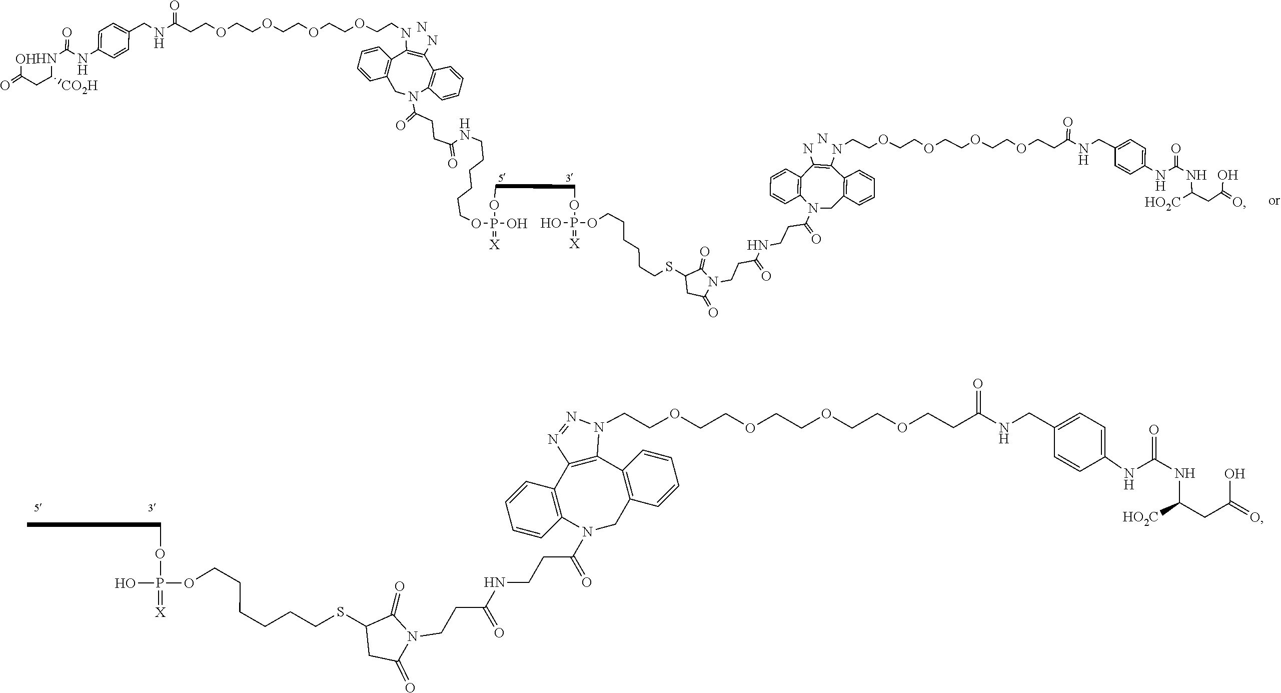

wherein X is O or S.

Embodiment 109. The compound, or a stereoisomer, tautomer, prodrug, or salt thereof, of any one of embodiments 105-108, wherein X is O.

Embodiment 110. The compound, or a stereoisomer, tautomer, prodrug, or salt thereof, of any one of embodiments 105-108, wherein X is S.

Embodiment 111. The compound, or a stereoisomer, tautomer, prodrug, or salt thereof, of any one of embodiments 1-110, wherein R¹ comprises an oligonucleotide.

Embodiment 112. The compound, or a stereoisomer, tautomer, prodrug, or salt thereof, of embodiment 111, wherein the oligonucleotide is attached at its 5′ end.

Embodiment 113. The compound, or a stereoisomer, tautomer, prodrug, or salt thereof, of embodiment 111, wherein the oligonucleotide is attached at its 3′ end.

Embodiment 114. The compound, or a stereoisomer, tautomer, prodrug, or salt thereof, of embodiment 111, wherein the oligonucleotide is attached at an internal position on the oligonucleotide.

Embodiment 115. The compound, or a stereoisomer, tautomer, prodrug, or salt thereof, of embodiment 114, wherein the internal position is an internucleoside linkage.

Embodiment 116. The compound, or a stereoisomer, tautomer, prodrug, or salt thereof, of any one of embodiments 1-115, wherein R¹ comprises an oligonucleotide conjugated to one or more additional α₄β_{1/7} ligands.

Embodiment 117. The compound, or a stereoisomer, tautomer, prodrug, or salt thereof, of embodiment 116, wherein the oligonucleotide is conjugated to two, three, four, five, or more than five additional α₄β_{1/7} ligands.

Embodiment 118. The compound, or a stereoisomer, tautomer, prodrug, or salt thereof, of embodiment 116 or 117, wherein the additional α₄β_{1/7} ligands are conjugated to the oligonucleotide at the 5′ end of the oligonucleotide, the 3′ end of the oligonucleotide, one or more internal positions on the oligonucleotide, or any combination thereof.

Embodiment 119. The compound, or a stereoisomer, tautomer, prodrug, or salt thereof, of any one of embodiments 111-119, wherein the oligonucleotide is a modified oligonucleotide.

Embodiment 120. A composition comprising a compound, or a stereoisomer, tautomer, prodrug, or salt thereof, of any one of embodiments 1-119, and a pharmaceutically acceptable excipient.

Embodiment 121. A method for delivering a therapeutic oligonucleotide to the brain of a subject, comprising administration of a compound, or a stereoisomer, tautomer, prodrug, or salt thereof, of any one of embodiments 1-119, or a composition of embodiment 120, to the subject.

Embodiment 122. The method of embodiment 121, wherein the therapeutic oligonucleotide is delivered to one or more brain regions selected from the group consisting of the striatum, the cerebellum, the brain stem, the hippocampus, the frontal cortex, and the spinal cord.

Embodiment 123. A method for treating or ameliorating a disease, disorder, or symptom thereof in a subject, comprising administration of a compound, or a stereoisomer, tautomer, prodrug, or salt thereof, of any one of embodiments 1-119, or a composition of embodiment 120, to the subject.

Embodiment 124. The method of embodiment 123, wherein the disease, disorder, or symptom thereof is a central nervous system (CNS) disease, disorder, or symptom thereof.

Embodiment 125. The method of embodiment 123 or 124, wherein the disease, disorder, or symptom thereof is Alzheimer's disease, or a symptom thereof.

Embodiment 126. The method of any one of embodiments 123-125, wherein the compound, or a stereoisomer, tautomer, prodrug, or salt thereof, is administered to the subject intrathecally.

Embodiment 127. A method for making a compound, or a stereoisomer, tautomer, prodrug, or salt thereof, of any one of embodiments 1-119, comprising one or more compounds and chemical transformations described herein, including Examples 1-13.

### EXAMPLES

In order that the embodiments described herein may be more fully understood, the following examples are set forth. The examples described in this application are offered to illustrate the compounds, compositions, and methods provided herein and are not to be construed in any way as limiting their scope.

### General Experimental Procedures

Definitions of variables in the structures in schemes herein are commensurate with those of corresponding positions in the formulae delineated herein.

### Common Abbreviations

ACN acetonitrile
br broad
CDI carbonyldiimidazole
d doublet
DCM dichloromethane
dd doublet of doublets
dba dibenzylideneacetone
DBCO Azadibenzocyclooctyne
DFAA difluoroacetic anhydride
DIPEA or DIEA diisopropylethylamine
DMF dimethylformamide
DMSO dimethyl sulfoxide
dppf 1,1′-ferrocenediyl-bis(diphenylphosphine)
EDCI 1-ethyl-3-(3-dimethylaminopropyl)carbodiimide
EtOAc ethyl acetate
h hour(s)
HBTU hexafluorophosphate benzotriazole tetramethyl uronium
HRMS high resolution mass spectrometry
HPLC high performance liquid chromatography
LCMS liquid chromatography and mass spectrometry
MS mass spectrometry
MW microwave
m multiplet
MeOH methanol
min minutes
mL milliliter(s)
MWCO molecular weight cut off
m/z mass to charge ratio
NMP N-methyl-2-pyrrolidone
NMR nuclear magnetic resonance
ppm parts per million
rt or RT room temperature
s singlet
t triplet
TFA trifluoroacetic acid
TFAA trifluoroacetic anhydride
TLC thin layer chromatography

### Example 1A-1B: Synthesis of α₄β_{1/7} integrin ligand-conjugated oligonucleotides

Exemplary compounds falling within the scope of the present disclosure can be synthesized according to the following procedures:

### Example 1A

General Procedure I: 5′-conjugated sense strand

Step 1: To an aqueous solution of 5′-amine functionalized sense strand (I) was added 10% V/V 1M Sodium Phosphate buffer (pH=7) and 20% -50% V/V CH₃CN. A solution of DBCO-NHS (II) (1.5-3 eq) in DMSO or CH₃CN was added to the reaction. The reaction is monitored by LCMS and HPLC. Upon completion, precipitate was removed using centrifugation and the aqueous solution was purified by reverse phase HPLC. The product fractions were combined and dried by lyophilization. The dried N-DBCO modified sense strand (III) was reconstituted in RNase free water for step 2.

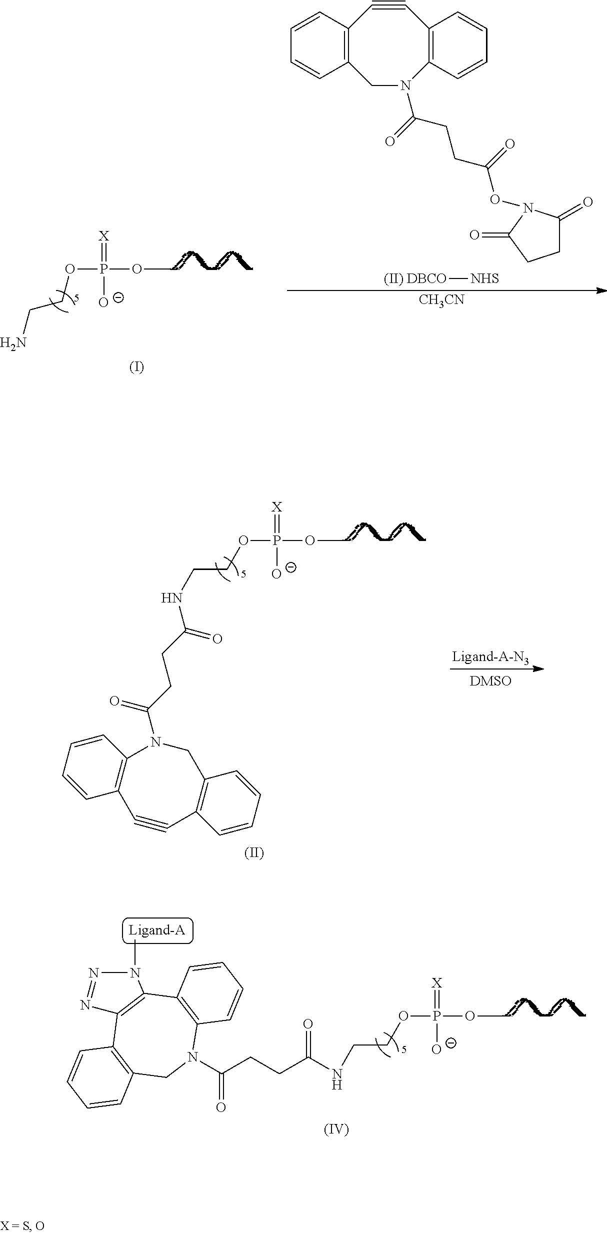

Step 2: To a solution of 5′-DBCO modified sense strand (III) (1 eq) was added a solution of ligand-A-N₃ (2 eq) in DMSO or THF. The reaction was monitored by HPLC and LCMS. Upon completion, the 5′-conjugated sense strand (IV) was purified by reverse phase HPLC or molecular weight cut-off with Amicon* Ultra-15 Centrifugal filter (3K, 5 times).

General Procedure II Type A: bis-conjugated sense strand

Step 1: To an aqueous solution of 5′ amine functionalized sense strand (I) was added 10% V/V 1M Sodium Phosphate buffer (pH=7) and 20% -50% V/V CH₃CN. A solution of DBCO-NHS (II) (1.5-3 eq) in DMSO or CH₃CN was added to the reaction. The reaction was monitored by LCMS and HPLC. Upon completion, precipitate was removed by centrifugation and the aqueous solution was purified by reverse phase HPLC. The product fractions were combined and dried by lyophilization. The 5′-DBCO modified sense strand (III) was reconstituted in RNase free water for step 2.

Step 2: To an aqueous solution of 5′-DBCO modified, 3′-(C₆-SS-C₆)-mC functionalized sense strand (III) was added 10% V/V 1M Sodium Phosphate buffer (pH=7). Tris(2-carboxyethyl)phosphine hydrochloride (TCEP) (25 eq) was dissolved in H2O and the PH of the solution was adjusted to 7 using 10 M NaOH. The aqueous TCEP solution was added to the solution of sense strand (III). The reaction was monitored by HPLC and LCMS. Upon completion, excess TCEP was removed by MWCO with 100 mM sodium phosphate buffer (pH=7) (3x). To the disulfide-reduced sense strand solution was added a solution of DBCO-MAL (V) (3 eq) in DMSO. The reaction was monitored by LCMS and HPLC. Upon completion, the solid was removed by centrifugation and the solution was purified by reverse phase HPLC and dried by lyophilization. The dried bis-DBCO modified sense strand (VI) was reconstituted in RNase free water for step 3.

Step 3: To a solution of 5′-, 3′-DBCO functionalized sense strand (VI) (1 eq) was added a solution of ligand-A-N₃ (3 eq) in DMSO or THF. The reaction was monitored by HPLC and LCMS. Upon completion, the 5′-, 3′-bis-conjugated sense strand (VII) was purified by reverse phase HPLC or molecular weight cut-off with Amicon* Ultra-15 Centrifugal filter (3K, 5 times). The product was confirmed by HPLC and LCMS.

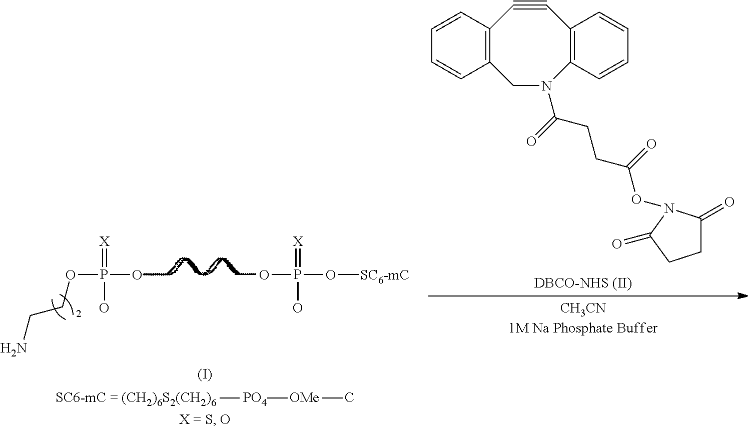

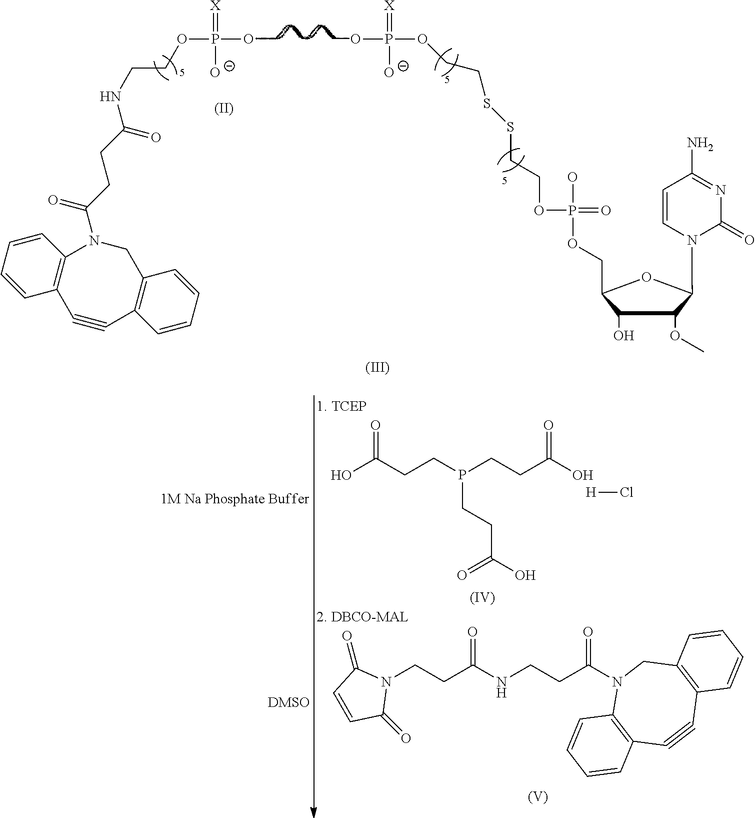

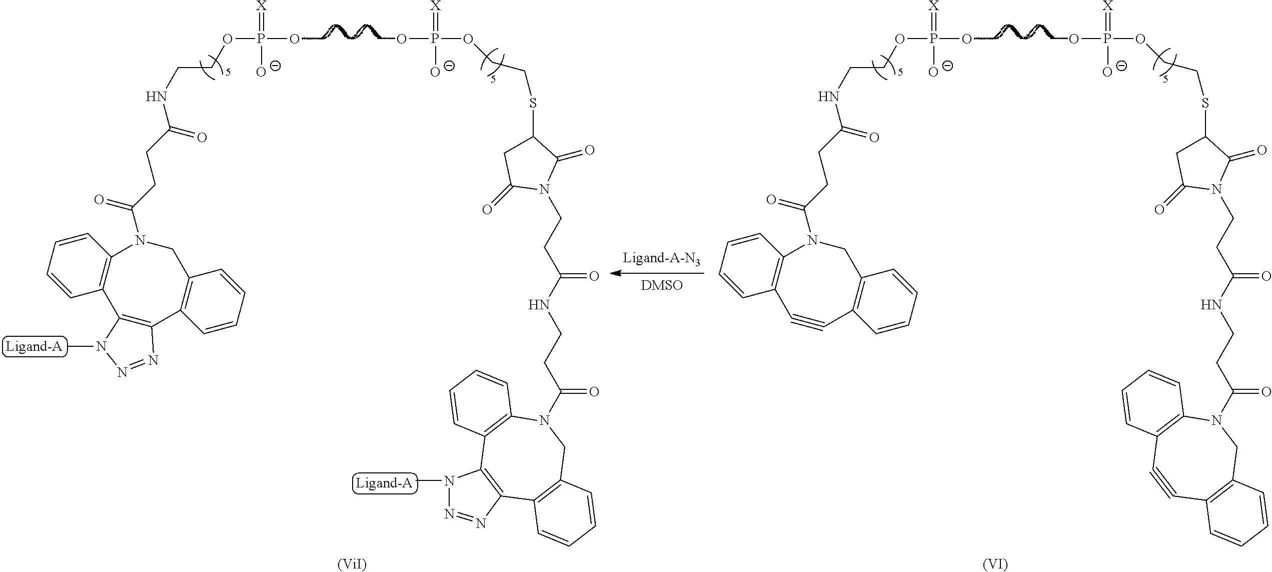

General Procedure II Type B: 3′,5′-conjugated sense strand

Step 1: To an aqueous solution of 5′-amine, 3′-(C₆-SS-C₆)-mC functionalized sense strand (I) was added 10% V/V 1M Sodium Phosphate buffer (pH=7) and 20% -50% V/V CH₃CN. A solution of DBCO-NHS (II) (1.5-3 eq) in DMSO or CH₃CN was added to the reaction. The reaction was monitored by LCMS and HPLC. Upon completion, precipitate was removed by centrifugation and the aqueous solution was purified by reverse phase HPLC. The combined product fractions were collected and dried by lyophilization. The 5′-DBCO modified sense strand (III) was reconstituted in RNase free water for step 2.

Step 2: To an aqueous solution of 5′-DBCO modified sense strand (III) (1 eq) was added a solution of ligand-A-N₃ (2 eq) in DMSO. The reaction is monitored by HPLC and LCMS. Upon completion, the 5′-conjugated sense strand (IV) was purified by reverse phase HPLC or molecular weight cut-off with Amicon* Ultra-15 Centrifugal filter (3K, 5 times).

Step 3: To a solution of 5′-conjugated, 3′-(C₆-SS-C₆)-mC functionalized sense strand (IV) (1 eq) in H₂O was added 10% V/V 1M sodium phosphate buffer (pH=7). TCEP (V) (20 eq) was dissolved in H₂O and the PH of the solution was adjusted to 7 using 10 M NaOH. The aqueous TCEP solution was added to the solution of sense strand (IV). The reaction was monitored by HPLC and LCMS. Upon completion, excess TCEP was removed with 100 mM sodium phosphate buffer (pH=7) using MWCO (3x). A solution of DBCO-MAL (VI) (3 eq) in DMSO was added to the disulfide-reduced sense strand solution. The reaction was monitored by HPLC and LCMS. Upon completion, the aqueous solution was purified by reverse phase HPLC. The product fractions were collected and dried by lyophilization. The 5′-conjugated, 3′-DBCO modified sense strand (VII) was reconstituted in 100 mM Sodium Phosphate buffer for the next step.

Step 4: To an aqueous solution of 5′-conjugated, 3′-DBCO functionalized sense strand (VII) (1 eq) was added a solution of ligand-B-N₃ (2 eq) in DMSO. The reaction was monitored by HPLC and LCMS. Upon completion, the 5′-, 3′-conjugated sense strand (VIII) was purified by reverse phase HPLC or molecular weight cut-off with Amicon* Ultra-15 Centrifugal filter (3K, 5 times).

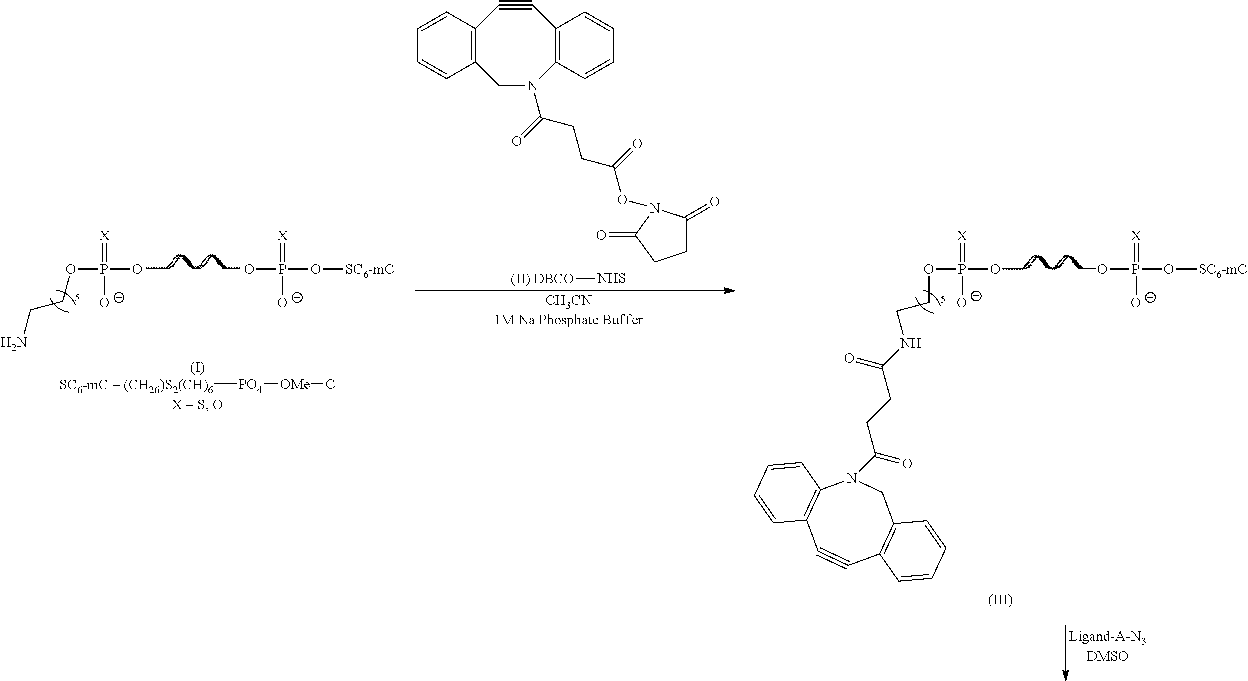

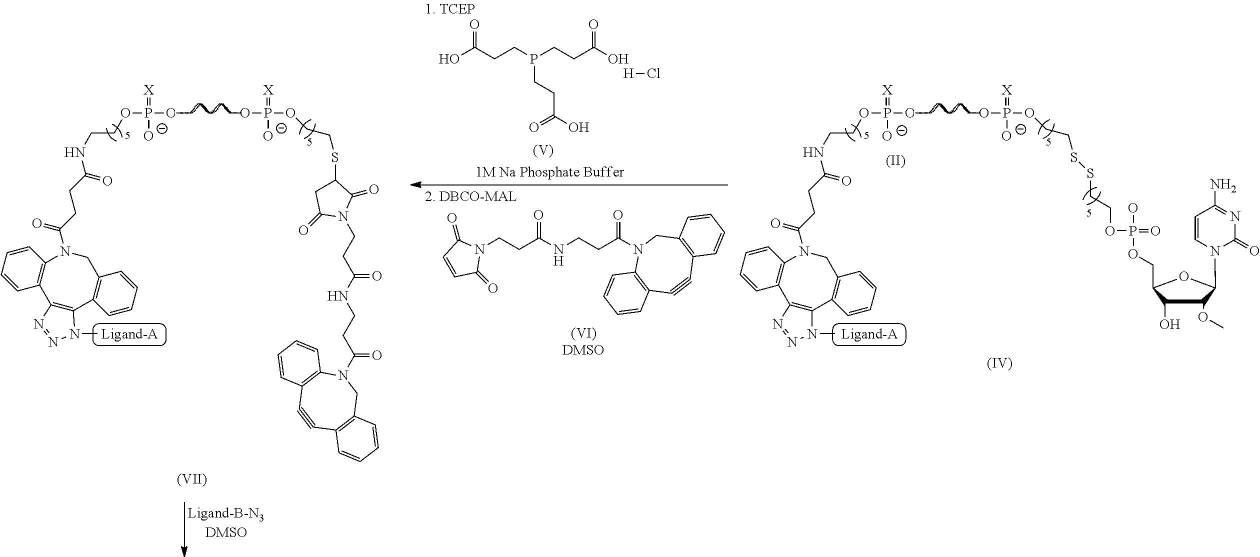

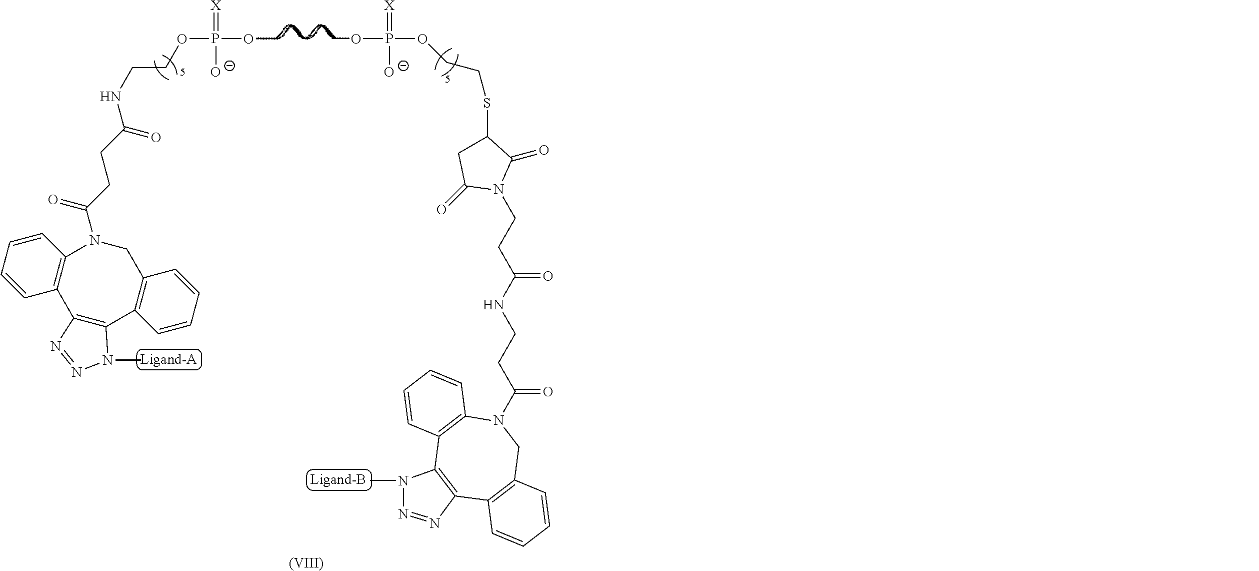

General Procedure III: 3′-conjugated sense strand

Step 1: To an aqueous solution of 3′-(C₆-SS-C₆)-mC functionalized sense strand (I) was added 10% V/V 1M Sodium Phosphate buffer (pH=7). TCEP (II) (20 eq) was dissolved in H₂O and the PH of the solution was adjusted to 7 using 10 M NaOH. The aqueous TCEP solution was added to the solution of sense strand (I). The reaction was monitored by HPLC and LCMS. Upon completion, excess TCEP was removed by MWCO with 100 mM sodium phosphate buffer (pH=7) (3x). To the disulfide-reduced sense strand solution was added a solution of DBCO-MAL (III) (3 eq) in DMSO. The reaction was monitored by LCMS and HPLC. Upon completion, the solid was removed by centrifugation and the solution was purified by reverse phase HPLC. The product fractions were collected and dried by lyophilization. The dried 3′-DBCO modified sense strand (IV) was reconstituted in 100 mM Sodium Phosphate buffer for step 3.

Step 3: To an aqueous solution of 3′-DBCO functionalized sense strand (IV) (1 eq) was added a solution of ligand-A-N₃ (3 eq) in DMSO. The reaction is monitored by HPLC and LCMS. Upon completion, the 3′-conjugated sense strand (V) was purified by reverse phase HPLC or molecular weight cut-off with Amicon* Ultra-15 Centrifugal filter (3K, 5x). The product was confirmed by HPLC and LCMS.

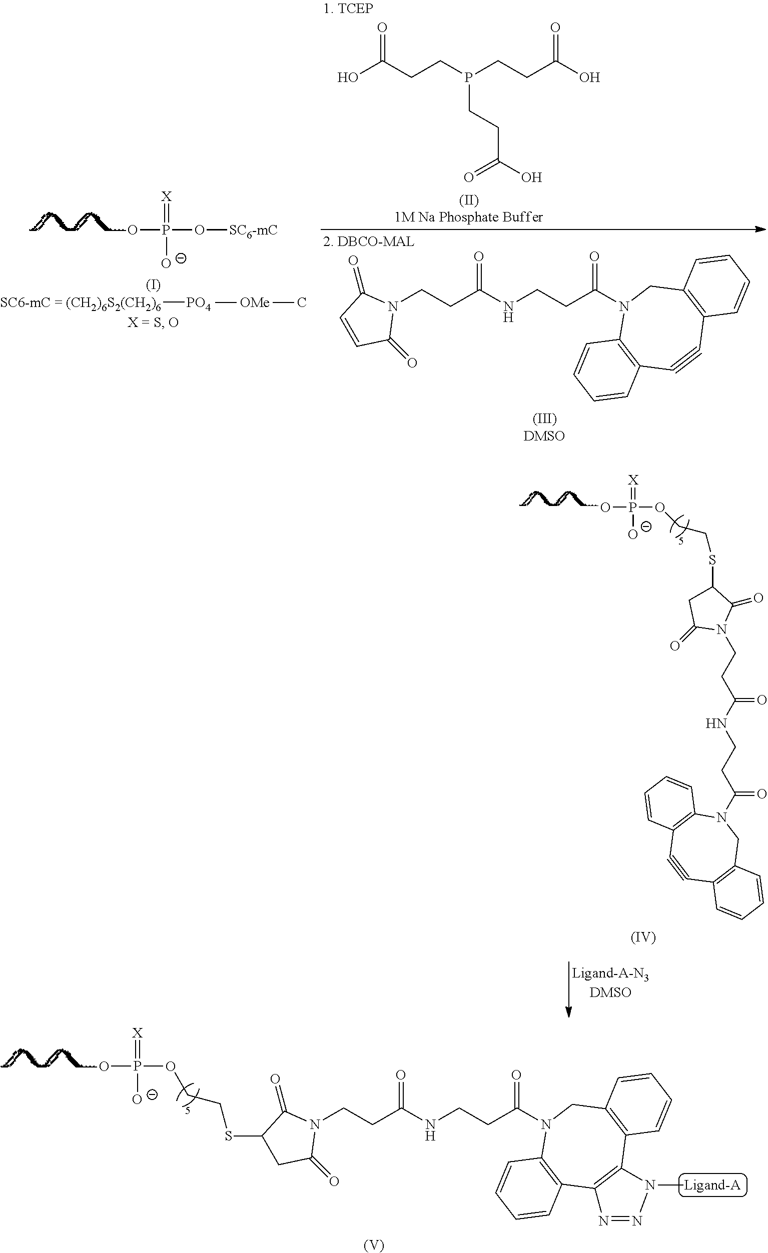

### Example 1B: Synthesis of α₄β_{1/7} integrin ligand-conjugated oligonucleotides General Scheme

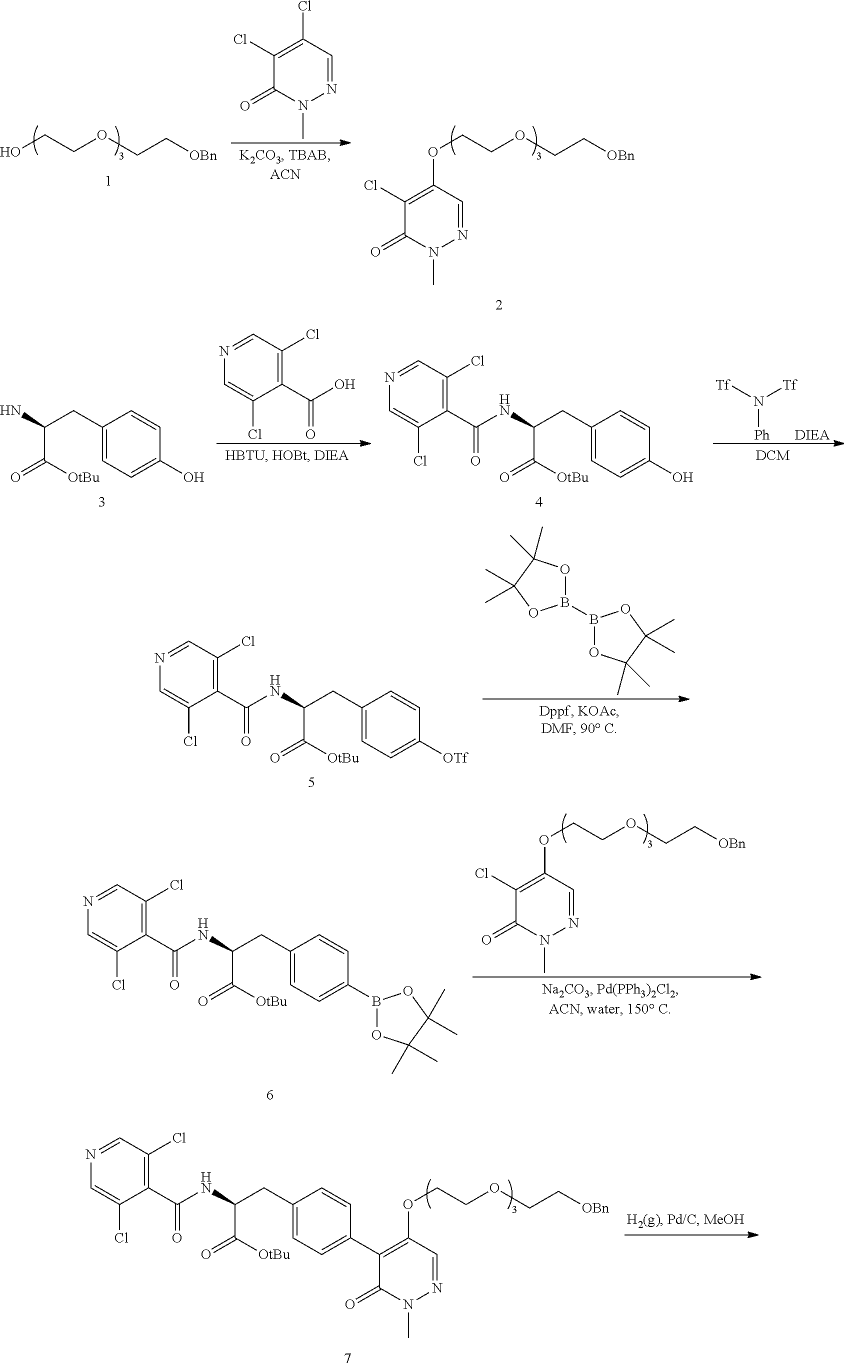

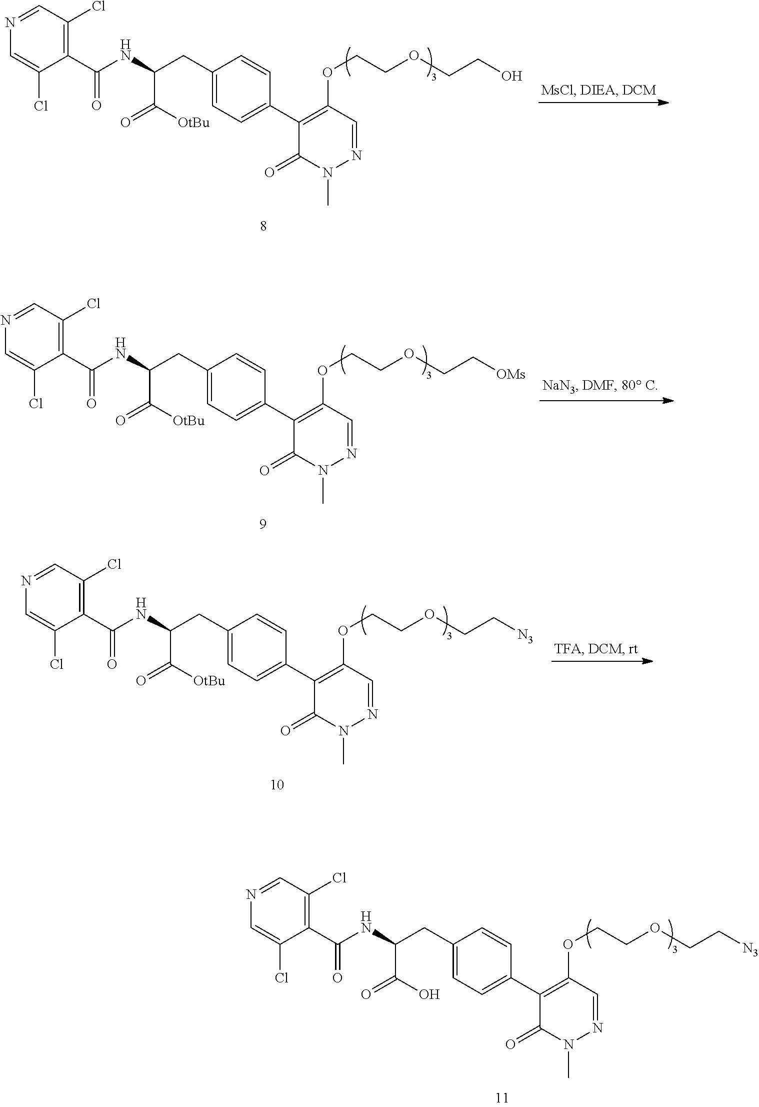

### Synthetic Procedures:

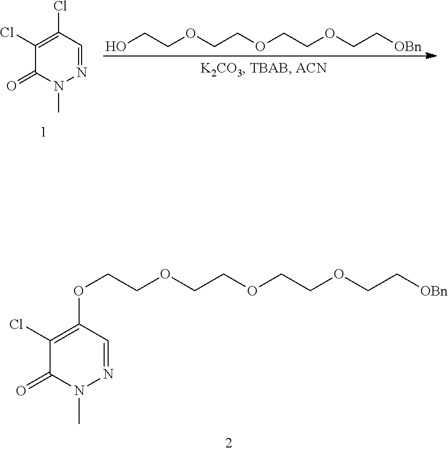

To a mixture of pyridazinone (2 g, 11.173 mmol, 1 eq), finely ground K₂CO₃ (6.177 g, 44.693 mmol, 4 eq) and Tetrabutylammonium bromide (0.36 g, 1.117 mmol, 0.1 eq) in dry acetonitrile (11 ml) was added BnO-(PEG)₄—OH (4.766 g, 16.76 mmol, 4.332 ml, 1.5 eq) over 10 mins at rt. The mixture was stirred overnight at rt, then the inorganics were filtered off, and the filtrate was concentrated under reduced pressure. The residue obtained was purified by reverse-phase column chromatography using a gradient 0-40% acetonitrile in water (+0.1% formic acid) to afford the desired product in 74% yield (3.52 g). MS ESI+m/z=449.0 [MNa]+.

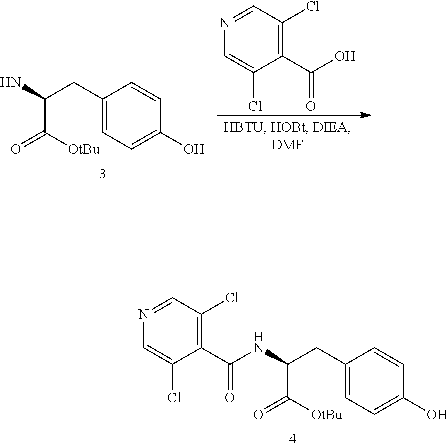 [SMILES: CC(C)(C)OC(=O)[C@@H](N)Cc1ccc(O)cc1 ; O=C(O)c1c(Cl)cncc1Cl ; CC(C)(C)OC(=O)[C@H](Cc1ccc(O)cc1)NC(=O)c1c(Cl)cncc1Cl]

To a solution of 3,5-Dichloroisonicotinic acid (7 g, 36.458 mmol, 1 eq) in DMF (102 ml) were successively added HBTU (15.209 g, 40.104 mmol, 1.1 eq) and HOBt (1.159 g, 7.292 mmol, 0.2 eq), followed by DIEA (14.137 g, 109.375 mmol, 19.104 ml, 3 eq) dropwise at 0° C. The solution was stirred for 20 minutes, then tert-butyl tyrosine (8.219 g, 34.635 mmol, 0.95 eq) was added, and the reaction was stirred at room temp for 3 h. The reaction was taken up in EtOAc and washed with water, then brine. The EtOAc layer was dried, filtered, and concentrated. The residue obtained was purified by silica-gel column chromatography using a gradient 0-55% EtOAc in hexanes to afford the desired product in 38% yield (5.72 g). MS ESI+m/z=411.4 [MH]+.

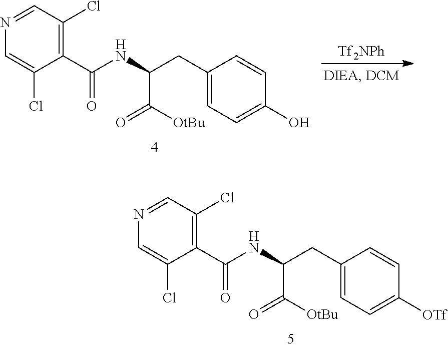 [SMILES: "Tf2NPh" ; CC(C)(C)OC(=O)[C@H](Cc1ccc(O)cc1)NC(=O)c1c(Cl)cncc1Cl ; CC(C)(C)OC(=O)[C@H](Cc1ccc(OS(=O)(=O)C(F)(F)F)cc1)NC(=O)c1c(Cl)cncc1Cl]

To a solution of Phenol (5.42 g, 13.178 mmol, 1 eq) in DCM (14.5 mL) was added Tf₂NPh (6.12 g, 17.132 mmol, 1.3 eq) followed by DIEA (5.1 g, 39.535 mmol, 6.892 ml, 3 eq) at 0° C. The mixture was stirred at rt for 15 h. The reaction mixture was concentrated under reduced pressure, diluted with water, and extracted with EtOAc. The combined organic layers were washed with aqueous saturated NaHCO₃ and brine, dried over Na₂SO₄, filtered, and then concentrated under reduced pressure. The residue obtained was purified by silica-gel column chromatography using a gradient 0-20% ethyl acetate in hexanes to afford the desired product in 87% yield (6.2 g). MS ESI+m/z=543.3 [MH]+.

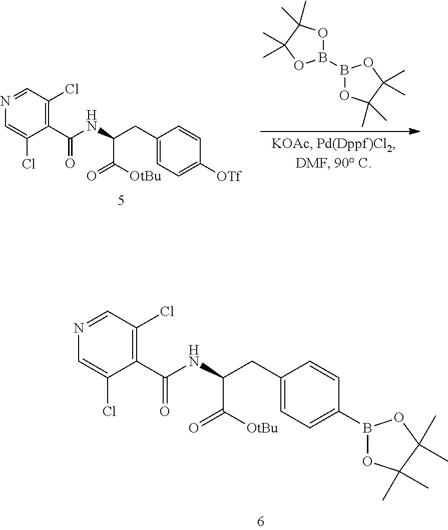

A solution of triflate (3.3 g, 6.07 mmol, 1 eq), diboron pinacol (2.31 g, 9.11 mmol, 1.5 eq) and KOAc (2.08 g, 21.25 mmol, 3.5 eq) in anhydrous DMF (33. ml) was degassed by sparging argon under stirring for 10 mins. Pd(Dppf)Cl₂ (889 mg, 1.21 mmol, 0.2 eq) was added under argon, and the mixture was degassed for another 5 mins. The vial was sealed and stirred at 90° C. for 10 h. The mixture was cooled down, and the catalyst was filtered off using celite. The filtrate was concentrated, and the residue obtained was partitioned between EtOAc and brine. The organic phase was dried, filtered, concentrated, and then purified by silica-gel column chromatography using 0-30% EtOAc in hexanes to afford the desired product in 84% yield (2.67 g). MS ESI+m/z=521.5 [MH]+.

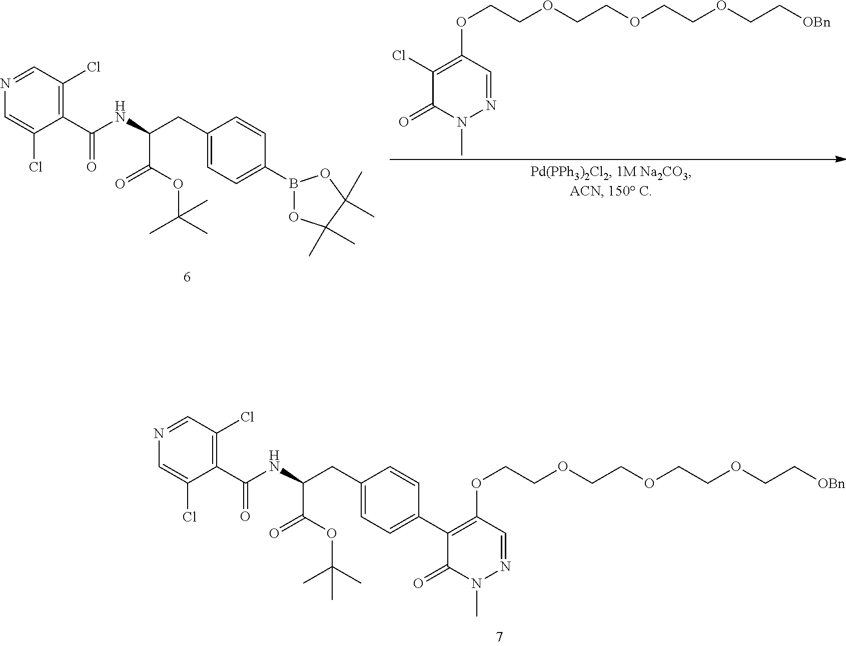 [SMILES: CC(C)(C)OC(=O)[C@H](Cc1ccc(B2OC(C)(C)C(C)(C)O2)cc1)NC(=O)c1c(Cl)cncc1Cl ; Cn1ncc(OCCOCCOCCOCCOCc2ccccc2)c(Cl)c1=O ; Cn1ncc(OCCOCCOCCOCCOCc2ccccc2)c(-c2ccc(C[C@H](NC(=O)c3c(Cl)cncc3Cl)C(=O)OC(C)(C)C)cc2)c1=O]

A mixture composed of Boronic ester (2.2 g, 4.221 mmol, 1 eq), Aryl chloride (1.802 g, 4.221 mmol, 1 eq) and 1M Na₂CO₃ (1.118 g, 10.552 mmol, 2.5 eq) in acetonitrile (10.5 ml) was degassed by sparging argon for 10 mins under stirring. Pd(PPh₃)₂C₁₂ (0.593 g, 0.844 mmol, 0.2 eq) was added, and the mixture was degassed for another 5 mins. The vial was sealed, stirred at 150° C. for 4 h, and then cooled down to rt. The catalyst was filtered off through celite, and the filtrate was concentrated. The residue obtained was purified by reverse-phase column chromatography (C18) using a gradient 0-60% acetonitrile in water (+0.1% formic acid) to afford the desired product in 53% yield (1.75 g). MS: ESI+m/z=785.6 [MH]+.

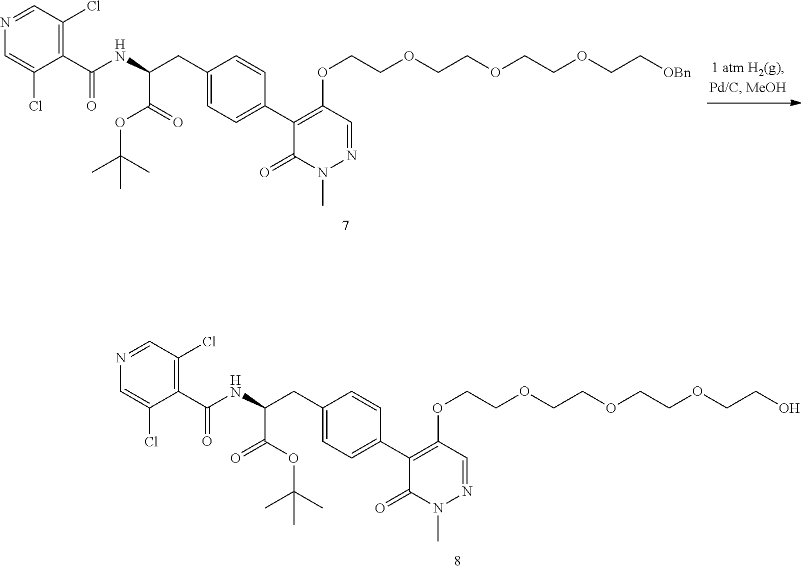 [SMILES: Cn1ncc(OCCOCCOCCOCCOCc2ccccc2)c(-c2ccc(C[C@H](NC(=O)c3c(Cl)cncc3Cl)C(=O)OC(C)(C)C)cc2)c1=O ; Cn1ncc(OCCOCCOCCOCCO)c(-c2ccc(C[C@H](NC(=O)c3c(Cl)cncc3Cl)C(=O)OC(C)(C)C)cc2)c1=O]

A solution of Benzyl ether (308 mg, 0.39 mmol) in methanol (6.5 ml) was stirred under 1 atm of H2(g) in the presence of Pd/C (42 mg, 0.39 mmol, 1 eq) for 6.5 h. The catalyst was filtered off, and the filtrate was concentrated. The residue obtained was purified by silica-gel column chromatography using a gradient of 0-5% methanol in ethyl acetate to afford the desired product in 77% yield (209 mg). MS ESI+m/z=695.5 [MH]+.

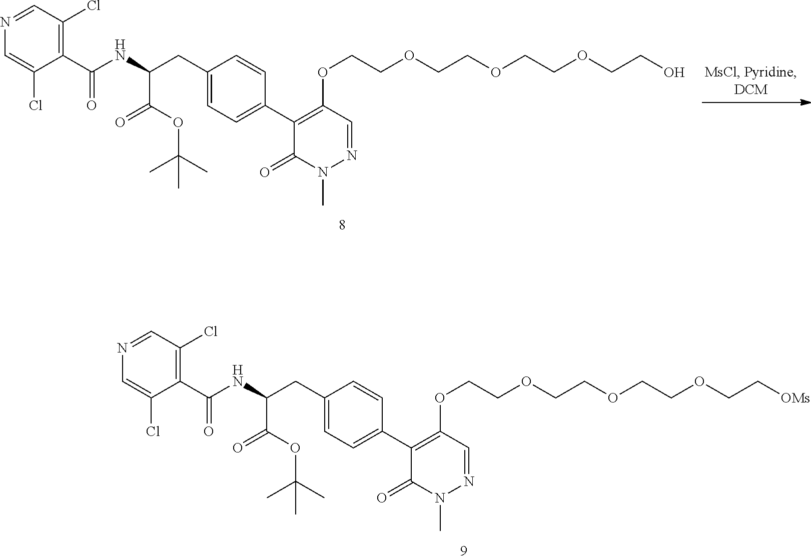 [SMILES: Cn1ncc(OCCOCCOCCOCCO)c(-c2ccc(C[C@H](NC(=O)c3c(Cl)cncc3Cl)C(=O)OC(C)(C)C)cc2)c1=O ; Cn1ncc(OCCOCCOCCOCCOS(C)(=O)=O)c(-c2ccc(C[C@H](NC(=O)c3c(Cl)cncc3Cl)C(=O)OC(C)(C)C)cc2)c1=O]

MsCl (0.145 g, 1.266 mmol, 0.098 ml, 1.5 eq) in DCM (8.5 ml) was added dropwise to a solution of alcohol (0.587 g, 0.844 mmol, 1 eq) in pyridine (8.5 ml) at 0° C. The reaction mixture was stirred at room temperature for 16 h, then quenched by addition of water and extracted with DCM. The organic layer was washed with a saturated solution of NaHCO₃ and brine. The organic layer was dried, then concentrated. The residue obtained (649 mg, 99%) was used for the following step without further purification. MS ESI+m/z=773.4 [MH]+.

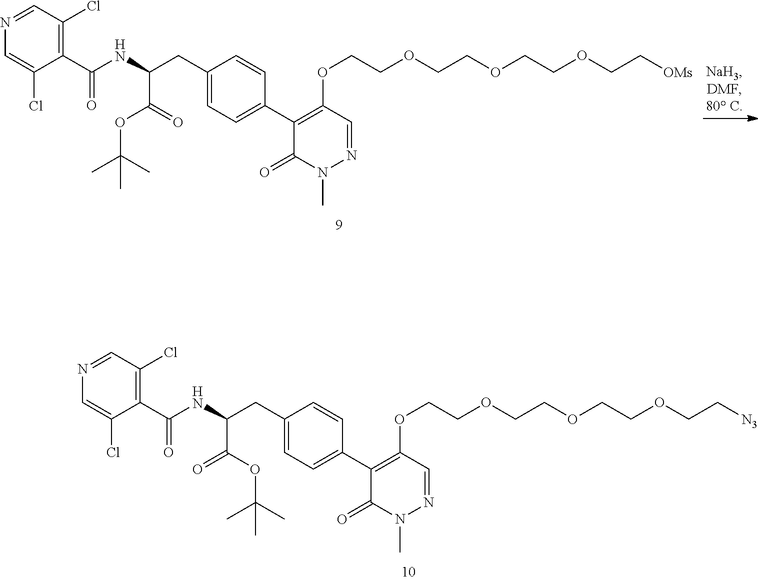 [SMILES: Cn1ncc(OCCOCCOCCOCCOS(C)(=O)=O)c(-c2ccc(C[C@H](NC(=O)c3c(Cl)cncc3Cl)C(=O)OC(C)(C)C)cc2)c1=O ; Cn1ncc(OCCOCCOCCOCCN=[N+]=[N-])c(-c2ccc(C[C@H](NC(=O)c3c(Cl)cncc3Cl)C(=O)OC(C)(C)C)cc2)c1=O]

To a solution of Mesylate (0.5 g, 0.646 mmol, 1 eq) in DMF (10 ml) was added NaN₃ (0.092 g, 1.422 mmol, 2.2 eq), and the mixture was stirred overnight at 80° C., then cooled down to rt and partitioned between EtOAc and water. The organic phase was separated, and the aqueous phase was extracted twice with ethyl acetate. The combined organic layers were washed with brine twice, dried, filtered, and concentrated. The residue obtained was purified by silica-gel column chromatography using a gradient 0-80% EtOAc in hexanes to afford the desired product in 67% yield (313 mg). MS ESI+m/z=720.5 [MH]+.

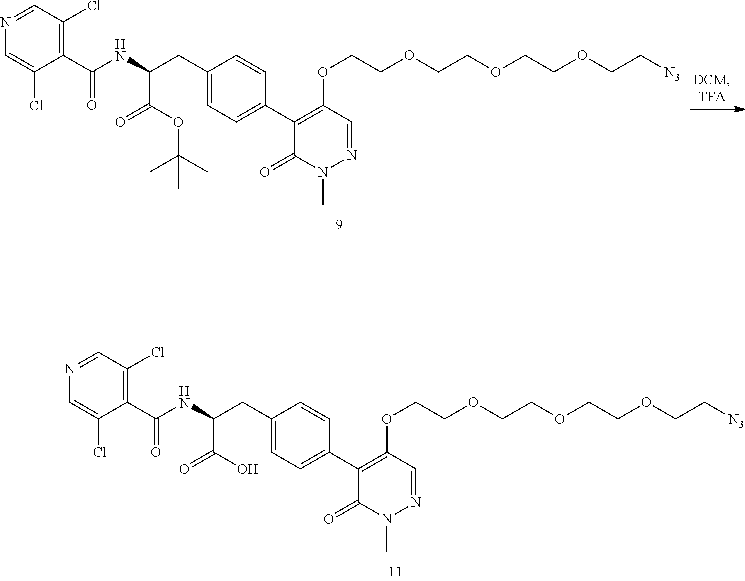 [SMILES: Cn1ncc(OCCOCCOCCOCCN=[N+]=[N-])c(-c2ccc(C[C@H](NC(=O)c3c(Cl)cncc3Cl)C(=O)OC(C)(C)C)cc2)c1=O ; Cn1ncc(OCCOCCOCCOCCN=[N+]=[N-])c(-c2ccc(C[C@H](NC(=O)c3c(Cl)cncc3Cl)C(=O)O)cc2)c1=O]

TFA (0.96 ml) was added dropwise at 0° C. to a solution of tert-butyl ester (0.17 g, 0.236 mmol, 1 eq) in DCM (1.9 ml). The cooling bath was removed, and the mixture was stirred overnight at rt, then concentrated under reduced pressure. The residue obtained was purified by C18 reverse-phase column chromatography using a gradient 0-100% acetonitrile in water (+0.1% formic acid). The residue obtained was re-purified by silica-gel column chromatography using a gradient of 0-20% methanol in EtOAc to afford the desired product in 44% yield (69 mg). MS ESI+m/z=664.1 [MH]+.

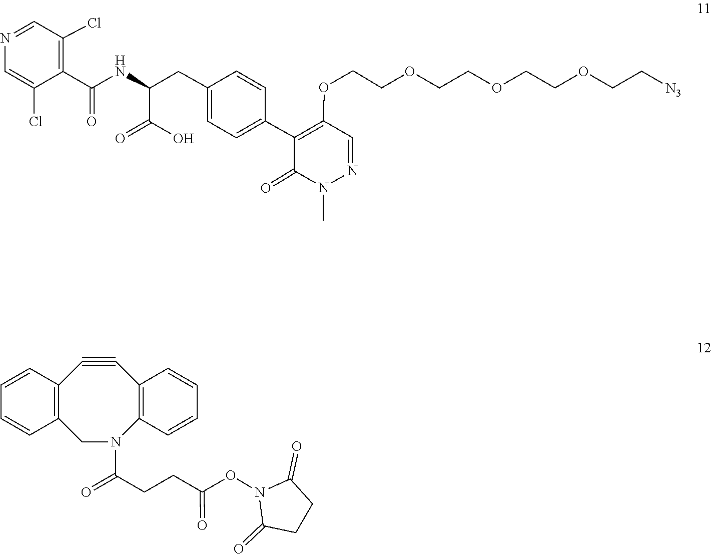 [SMILES: Cn1ncc(OCCOCCOCCOCCN=[N+]=[N-])c(-c2ccc(C[C@H](NC(=O)c3c(Cl)cncc3Cl)C(=O)O)cc2)c1=O ; O=C(CCC(=O)N1Cc2ccccc2C#Cc2ccccc21)ON1C(=O)CCC1=O]

To an aqueous solution of AC6 functionalized sense strand was added a solution of DCBO-NHS (12) (3 eq) in CH₃CN. The reaction was monitored by LCMS and HPLC. Once the reaction was complete, the solid was filtered off, and the solution was purified by HPLC and dried by lyophilization. The dried DCBO modified sense strand was reconstituted in RNase free water and ligand 11 (2 eq) in THF was added. After the reaction was complete, the conjugate was purified by MWCO (5X).

### General Procedure for Annealing:

The concentrations of both sense strand and antisense strand were determined by Nanodrop. The double-stranded siRNA was prepared by mixing equimolar of sense stand and antisense strand. The annealing process was monitored by RP-HPLC, non-denaturing method. After annealing, no more that 5% of antisense strand was in the duplex mixture. Duplex concentration was determined by measuring the solution absorbance on Nanodrop.

### Example 2: BA-128 Conjugates

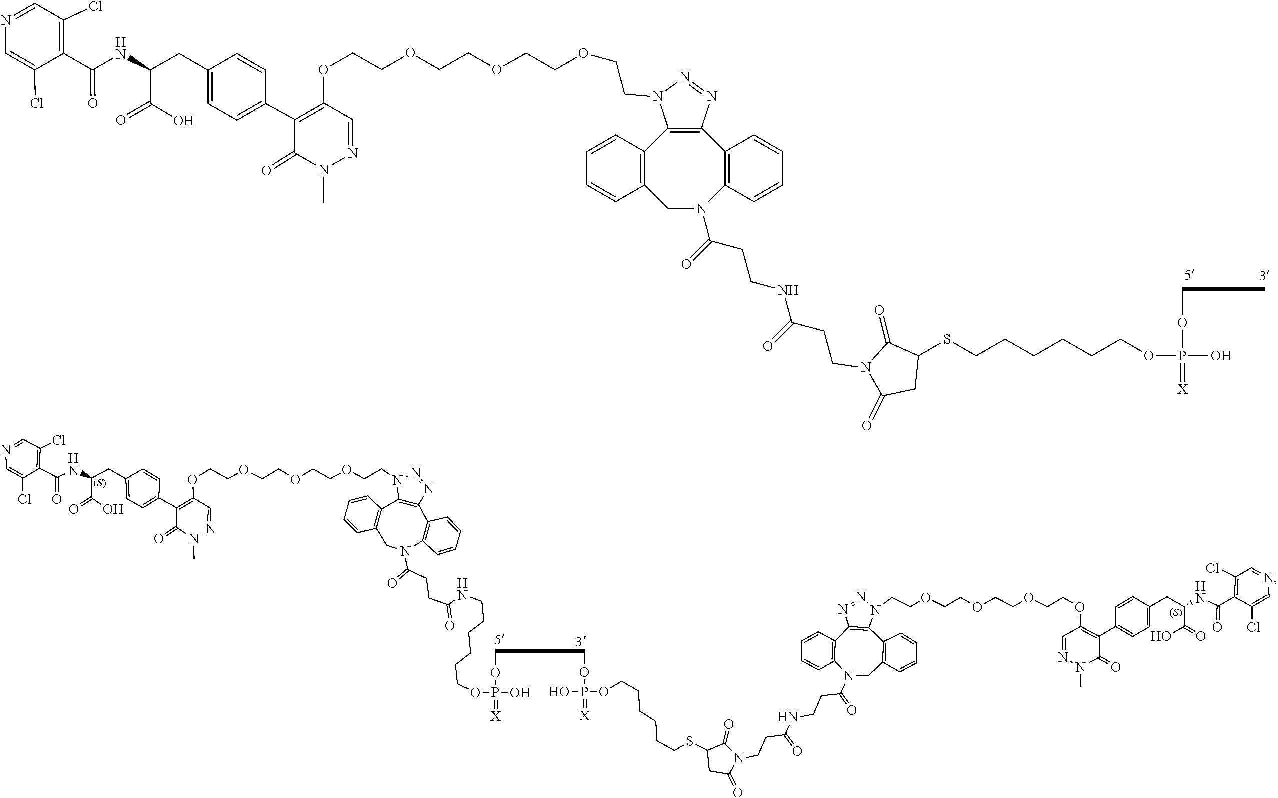

wherein X is O or S.

### BA-128: (S)-3-(4-(5-(2-(2-(2-(2-azidoethoxy)ethoxy)ethoxy)ethoxy)-2-methyl-3-oxo-2,3-dihydropyridazin-4-yl)phenyl)-2-(3,5-dichloroisonicotinamido)propanoic acid

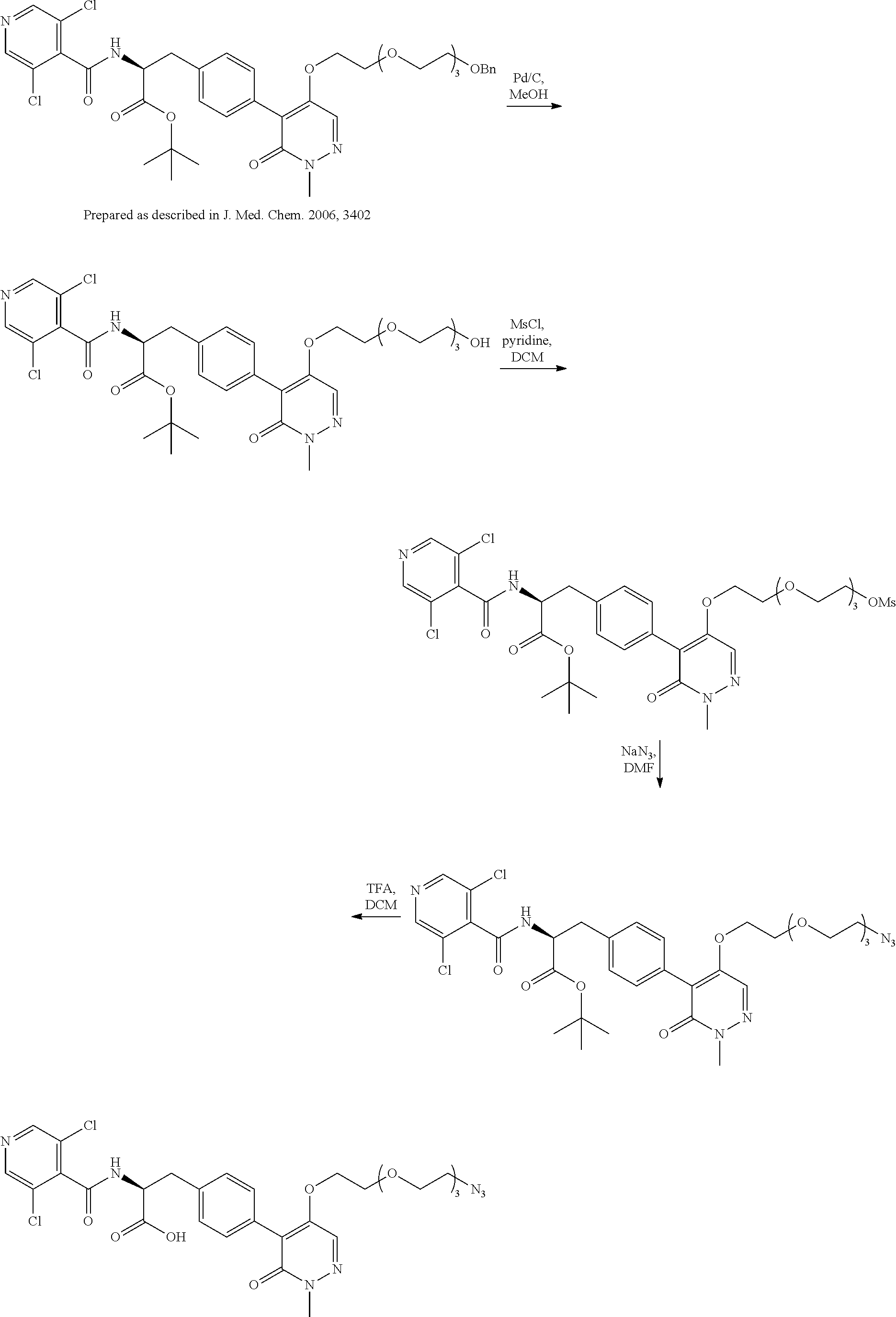

### Step 1: tert-butyl (S)-2-(3,5-dichloroisonicotinamido)-3-(4-(5-(2-(2-(2-(2-h. droxvetho) ethoxy)ethoxy)ethoxy)-2-methyl-3-oxo-2,3-dihydropyridazin-4-yl)phenyl)propanoate

A solution of tert-butyl (S)-2-(3,5-dichloroisonicotinamido)-3-(4-(2-methyl-3-oxo-5-((1-phenyl-2,5,8,11-tetraoxatridecan-13-yl)oxy)-2,3-dihydropyridazin-4-yl)phenyl)propanoate (0.81 g, 1.02 mmol) in MeOH (17 ml) was stirred under 1 atm of H₂(g) in the presence of Pd/C (0.11 g, 1.02 mmol) for 2 hours. The catalyst was filtered off and the filtrate was concentrated. The crude material was purified by silica gel column chromatography using a 0-5% MeOH in DCM to obtain the titled compound (0.47 g, 66%). MS (ESI) m/z 696.6 [M+H]⁺.

### Step 2: tert-butyl (S)-2-(3,5-dichloroisonicotinamido)-3-(4-(2-methyl-5-(2-(2-(2-(2-((methylsulfonyl)oxy)ethoxy)ethoxy)ethoxy)ethoxy)-3-oxo-2,3-dihydropyridazin-4-yl)phenyl)propanoate

Methanesulfonyl chloride (17 ul, 0.216 mmol) in DCM (1.4 ml) was added dropwise to a solution of tert-butyl (S)-2-(3,5-dichloroisonicotinamido)-3-(4-(5-(2-(2-(2-(2-hydroxyethoxy) ethoxy)ethoxy)ethoxy)-2-methyl-3-oxo-2,3-dihydropyridazin-4-yl)phenyl)propanoate (0.10 g, 0.144 mmol) in pyridine (1.4 ml) at 0° C. The reaction mixture was stirred at room temperature for 16 h then quenched by addition of water (10 ml) and extracted with DCM (3×10 ml). The organic layer was washed with a saturated solution of NaHCO₃ (10 ml) and brine (10 ml). The combined organic extracts were dried over anhydrous Na₂SO₄ and concentrated under reduced pressure to obtain the titled compound as a brown oil (0.11 g, quant.). MS (ESI) m/z 773.5 [M+H]⁺.

### Step 3: tert-butyl (S)-3-(4-(5-(2-(2-(2-(2-azidoethoxy)ethoxy)ethoxy)ethoxy)-2-methyl-3-oxo-2,3-dihydropyridazin-4-yl)phenyl)-2-(3,5-dichloroisonicotinamido)propanoate

To a solution of tert-butyl (S)-2-(3,5-dichloroisonicotinamido)-3-(4-(2-methyl-5-(2-(2-(2-(2-((methylsulfonyl)oxy)ethoxy)ethoxy)ethoxy)ethoxy)-3-oxo-2,3-dihydropyridazin-4-yl)phenyl)propanoate (0.11 mg, 0.143 mmol) in DMF (2.3 ml) was added NaN₃ (21 mg, 0.316 mmol) followed by 1 drop of water. The mixture was stirred for 6 hours at 80° C. then cooled down to room temperature and partitioned between EtOAc (20 ml) and water (20 ml). The organic phase was separated, and the aqueous phase was extracted twice with EtOAc (2×15 ml). The combined organic layers were washed with brine twice, dried, filtered, and concentrated. The obtained residue was purified by silicas-gel column chromatography using a gradient 0-100% EtOAc in hexane to afford the titled compound as a clear oil (85 mg, 82%). MS (ESI) m/z 721.6 [M+H]⁺.

### Step 4: (S)-3-(4-(5-(2-(2-(2-(2-azidoethoxy)ethoxy)ethoxy)ethoxy)-2-methyl-3-oxo-2,3-dihydropyridazin-4-yl)phenyl)-2-(3,5-dichloroisonicotinamido)propanoic acid

TFA (0.14 ml) was added dropwise at 0° C. to a solution of tert-butyl (S)-3-(4-(5-(2-(2-(2-(2-azidoethoxy)ethoxy)ethoxy)ethoxy)-2-methyl-3-oxo-2,3-dihydropyridazin-4-yl)phenyl)-2-(3,5-dichloroisonicotinamido)propanoate (25 mg, 0.035 mmol) in DCM (0.3 ml). The cooling bath was removed, and the mixture was stirred 17 hours at room temperature and concentrated under reduced pressure. The residue obtained was purified by silica-gel column chromatography using a gradient 0-100% methanol in DCM to afford the desired product (16 mg, 69%). MS (ESI) m/z 687.8 [M+Na]⁺. ¹H NMR (500 MHz, DMSO-d₆) δ 12.95 (s, 1H), 9.31 (d, J=10 Hz, 1H), 8.63 (s, 2H), 8.20 (s, 1H), 7.42 (d, J=10 Hz, 2H), 7.29 (d, J=10 Hz, 2H), 4.78-4.72 (m, 1H), 4.36-4.32 (m, 2H), 3.66 (s, 5H), 3.57 (t, J=5 Hz, 2H), 3.36 (t, J=5 Hz, 2H), 3.53-3.47 (m, 8H), 3.20 (dd, J=10, 5 Hz, 1H), 2.93 (q, J=10 Hz, 1H).

BA-128 was conjugated to an oligo sense strand according to general procedure type I. The product (MW: 8336.82 g/mol) was made with 98% purity and confirmed by HPLC and LCMS (m/z: 8335.56).

BA-128 was bis-conjugated to an oligo sense strand according to general procedure type JIB. The product (MW: 9483.37 g/mol) was made with 96% purity and confirmed with HPLC and LCMS (m/z: 9482.44).

### Example 3: BA-148 Conjugate

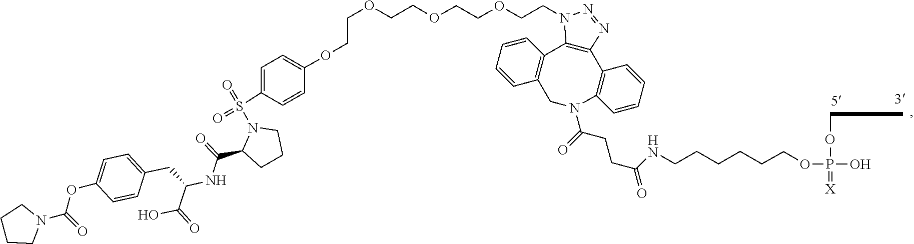

wherein X is O or S.

### BA-148: (S)-2-((S)-1-((4-(4-(2-(2-(azidooxy)ethoxy)ethoxy)butoxy)phenyl)sulfonyl)pyrrolidine-2-carboxamido)-3-(4-((pyrrolidine-1-carbonyl)oxy)phenyl)propanoic acid

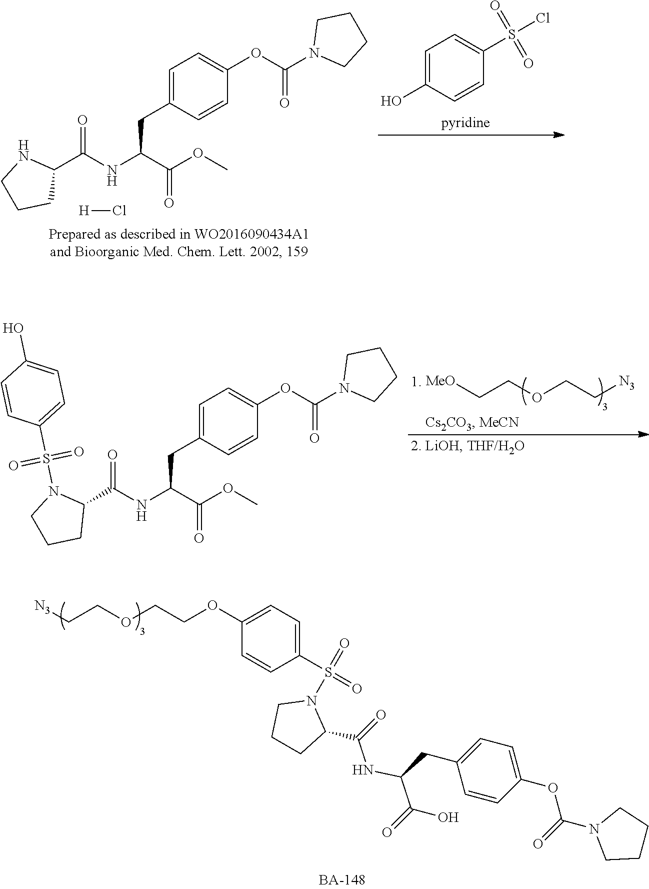

### Step 1: 4-((S)-2-((S)-1-((4-hydroxyphenyl)sulfonyl)pyrrolidine-2-carboxamido)-3-methoxy-3-oxopropyl)phenyl pyrrolidine-1-carboxylate

To a solution of 4-((S)-3-methoxy-3-oxo-2-((S)-pyrrolidine-2-carboxamido)propyl)phenyl pyrrolidine-1-carboxylate hydrochloride (0.43 g, 1 mmol) in pyridine (3 ml), 4-hydroxybenzenesulfonyl chloride (0.23 g, 1.2 mmol). The reaction mixture was stirred at room temperature under inert atmosphere for 17 hours. The excess pyridine was removed under vacuum. The residue was dissolved in EtOAc (60 ml) and washed with saturated ammonium chloride (20 ml), water and brine (10 ml). The organic layer was dried over anhydrous Na₂SO₄ and concentrated under reduced pressure. The crude material was purified by silica gel column chromatography using a gradient 50-100% EtOAc in hexane to afford the titled compound as a yellow oil (0.16 g, 29%). MS (ESI) m/z 546.2 [M+H]⁺.

### Step 2: 4-((S)-2-((S)-1-((4-(4-(2-(2-(azidooxy)ethoxy)ethoxy)butoxy)phenyl)sulfonyl)pyrrolidine -2-carboxamido)-3-methoxy-3-oxopropyl)phenyl pyrrolidine-1-carboxylate

To a solution of 4-((S)-2-((S)-1-((4-hydroxyphenyl)sulfonyl)pyrrolidine-2-carboxamido)-3-methoxy-3-oxopropyl)phenyl pyrrolidine-1-carboxylate (0.16 g, 0.293 mmol) in anhydrous MeCN (5 ml), 2-(2-(2-(2-azidoethoxy)ethoxy)ethoxy)ethyl methanesulfonate (0.11 g, 0.367 mmol), cesium carbonate (0.19 g, 0.587 mmol) were added. The reaction mixture was stirred at 60° C. under inert atmosphere for 16 hours. Reaction mixture was diluted with water (20 ml) and extracted with EtOAc (3×25 ml). The combined organic extracts were washed with brine (10 ml), dried over anhydrous Na₂SO₄ and concentrated under reduced pressure. The crude material was purified by silica gel column chromatography using a gradient 0-40% MeOH in EtOAc to afford the titled compound as a colorless oil (52 mg, 24%). MS (ESI) m/z 769.5 [M+Na]⁺.

### Step 3: (S)-2-((S)-1-((4-(4-(2-(2-(azidooxy)ethoxy)ethoxy)butoxy)phenyl)sulfonyl)pyrrolidine-2-carboxamido)-3-(4-((pyrrolidine-1-carbonyl)oxy)phenyl)propanoic acid

To a solution of 4-((S)-2-((S)-1-((4-hydroxyphenyl)sulfonyl)pyrrolidine-2-carboxamido)-3-methoxy-3-oxopropyl)phenyl pyrrolidine-1-carboxylate (50 mg, 0.70 mmol) in a mixture of THF (3 ml), MeOH (2 ml) and H₂O (0.5 ml), LiGH (11 mg, 0.27 mmol) was added and stirred at room temperature for 15 hours. The solvents were evaporated under vacuum. The residue was treated with 10% citric acid solution and purified by reverse-phase column chromatography (C18) using a gradient 0-40% ACN in water (+0.1% formic acid) to afford the titled compound as a white solid (43 mg, 88%). MS (ESI) m/z 755.9 [M+Na]⁺. ¹H NMR (400 MHz, DMSO-d₆) δ 7.81 -7.75 (m, 2H), 7.56 (m, 1H), 7.19-7.05 (m, 4H), 6.89 (dd, J=8.5, 3.5 Hz, 2H), 4.22-4.16 (m, 2H), 4.13-3.91 (m, 3H), 3.76 (dq, J=4.8, 2.4 Hz, 2H), 3.63-3.25 (m, 22H), 3.16-2.87 (m, 3H), 1.91-1.79 (m, 4H), 1.38 (m, 2H).

BA-148 was conjugated to an oligo sense strand according to general procedure type I. The product (MW: 8032.88 g/mol) was made with 98% purity and confirmed by HPLC and LCMS (m/z: 8031.37).

### Example 4: BA-149Conjugate

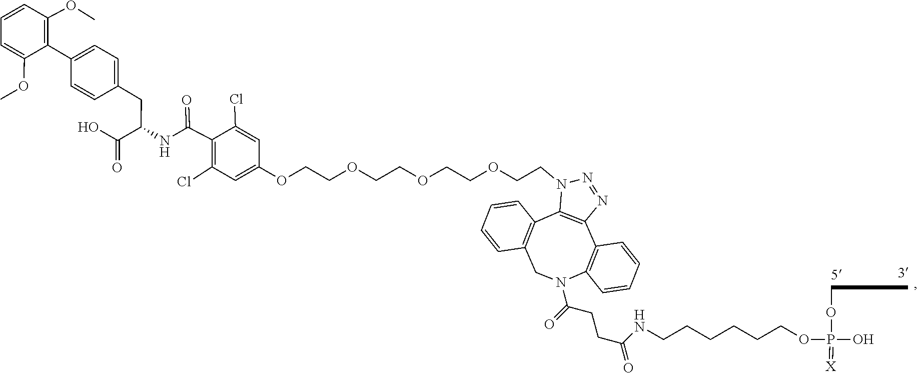

wherein X is O or S.

### BA-149: (S)-2-(4-(4-(2-(2-(azidooxy)ethoxy)ethoxy)butoxy)-2,6-dichlorobenzamido)-3-(2′,6′-dimethoxy-[1,1′-biphenyl]-4-yl)propanoic acid

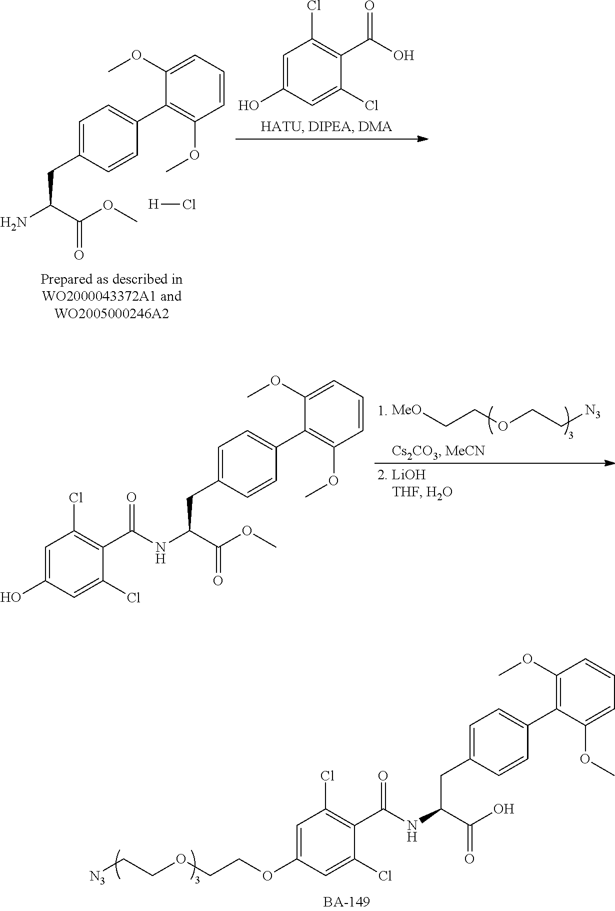

### Step 1: methyl (S)-2-(2,6-dichloro-4-hydroxybenzamido)-3-(2′,6′-dimethoxy-[1,1′-biphenyll-4-yl)propanoate

To a solution of methyl (S)-2-amino-3-(2′,6′-dimethoxy-[1,1′-biphenyl]-4-yl)propanoate (0.51 g, 1.45 mmol) in anhydrous DMA (3 ml), 2,6-dichloro-4-hydroxybenzoic acid (0.25 g, 1.2 mmol), HATU (0.69 g, 1.81 mmol) and DIPEA (0.42 ml, 2.42 mmol) were added. The reaction mixture was stirred at room temperature under inert atmosphere for 17 hours. Reaction mixture was diluted with water (200 ml) and a beige precipitate was formed. The solids were collected by filtration, washed with water, and air-dried to obtain the titled compound (0.38 g, 62%) as an off-white solid. MS (ESI) m/z 505.4 [M+H]⁺.

### Step 2: methyl (S)-2-(4-(4-(2-(2-(azidooxy)ethoxy)ethoxy)butoxy)-2,6-dichlorobenzamido)-3-(2′,6′-dimethoxy-[1,1′-biphenyll-4-yl)propanoate

To a solution of methyl (S)-2-(2,6-dichloro-4-hydroxybenzamido)-3-(2′,6′-dimethoxy-[1,1′-biphenyl]-4-yl)propanoate (0.19 g, 0.387 mmol) in anhydrous MeCN (5 ml), 2-(2-(2-(2-azidoethoxy)ethoxy)ethoxy)ethyl methanesulfonate (0.13 g, 0.425 mmol), cesium carbonate (0.25 g, 0.773 mmol) were added. The reaction mixture was stirred at 60° C. under inert atmosphere for 16 hours. Reaction mixture was diluted with water (20 ml) and extracted with EtOAc (3×25 ml). The combined organic extracts were washed with brine (10 ml), dried over anhydrous Na₂SO₄ and concentrated under reduced pressure. The crude material was purified by silica gel column chromatography using a gradient 50-100% EtOAc in hexane to afford the titled compound as a colorless oil (0.18 g, 65%). MS (ESI) m/z 706.7 [M+H]⁺.

### Step 3: (S)-2-(4-(4-(2-(2-(azidooxy)ethoxy)ethoxy)butoxy)-2,6-dichlorobenzamido)-3-(2′,6′-dimethoxy-[1,1′-biphenyll-4-yl)propanoic acid

To a solution of methyl (S)-2-(4-(4-(2-(2-(azidooxy)ethoxy)ethoxy)butoxy)-2,6-dichlorobenzamido)-3-(2′,6′-dimethoxy-[1,1′-biphenyl]-4-yl)propanoate (0.175 g, 0.248 mmol) in a mixture of THF (4 ml), MeOH (3 ml) and H₂O (0.5 ml), LiGH (41 mg, 1 mmol) was added and stirred at room temperature for 15 hours. The solvents were evaporated in vacuo. The residue was treated with 10% citric acid solution and purified by reverse-phase column chromatography (C18) using a gradient 0-40% ACN in water (+0.1% formic acid) to afford the titled compound as a clear colorless oil (96 mg, 56%). MS (ESI) m/z 692.6 [M+H]⁺. 1H NMR data (500 MHz, DMSO-d₆) δ 12.77 (s, 1H), 8.98 (d, J=8.3 Hz, 1H), 7.31-7.24 (m, 3H), 7.11-7.06 (m, 2H), 7.05 (s, 2H), 6.72 (d, J=8.5 Hz, 2H), 4.69 (ddd, J=10.4, 8.3, 4.4 Hz, 1H), 4.18-4.12 (m, 2H), 3.74-3.69 (m, 2H), 3.65 (s, 6H), 3.62-3.50 (m, 10H), 3.38 (dd, J=5.5, 4.3 Hz, 2H), 3.16 (dd, J=14.1, 4.4 Hz, 1H), 2.92 (dd, J=14.1, 10.5 Hz, 1H).

BA-149 was conjugated to an oligo sense strand according to general procedure type I. The product (MW: 7991.63 g/mol) was made with 87% purity and confirmed by HPLC and LCMS (m/z: 7990.46).

### Example 5: BA-154 Conjugate

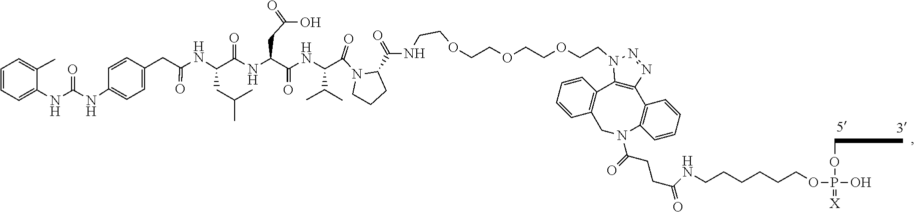

wherein X is O or S.

### BA-154: (S)-4-(((S)-1-((S)-2-((2-(2-(2-(2-azidoethoxy)ethoxy)ethoxy)ethyl)carbamoyl) pyrrolidin-1-yl)-3-methyl-1-oxobutan-2-yl)amino)-3-((S)-4-methyl-2-(2-(4-(3-(o-tolyl)ureido) phenyl)acetamido)pentanamido)-4-oxobutanoic acid

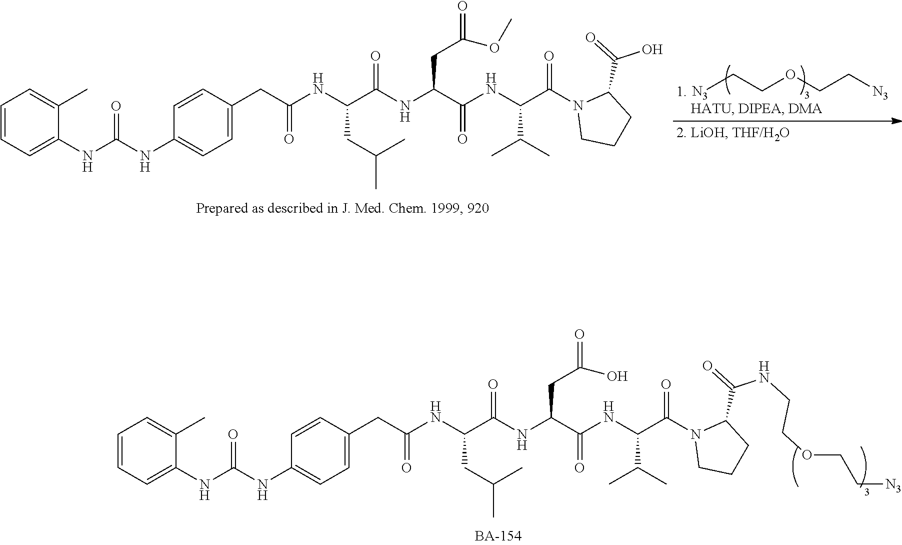

### Step 1: methyl (S)-4-(((S)-1-((S)-2-((4-(2-(2-(azidooxy)ethoxy)ethoxy)butyl)carbamoyl) pyrrolidin-1-yl)-3-methyl-1-oxobutan-2-yl)amino)-3-((S)-4-methyl-2-(2-(4-(3-(o-tolyl)ureido) phenyl)acetamido)pentanamido)-4-oxobutanoate

To a solution of ((S)-4-methoxy-2-((S)-4-methyl-2-(2-(4-(3-(o-tolyl)ureido)phenyl)acetamido) pentanamido)-4-oxobutanoyl)-L-valyl-L-proline (0.30 g, 0.415 mmol) in anhydrous DMA (5 ml), 4-(2-(2-(azidooxy)ethoxy)ethoxy)butan-1-amine (0.11 g, 0.519 mmol), HATU (0.32 g, 0.83 mmol) and DIPEA (0.22 mL, 1.25 mmol) were added. The reaction mixture was stirred at room temperature under inert atmosphere for 15 hours. Reaction mixture was diluted with water (150 ml) and an off-white precipitate was formed. The solids were collected by filtration, washed with water, and air-dried. The crude was redissolved in DCM and purified by silica gel column chromatography using a gradient 0-20% MeOH in EtOAc to afford the titled compound as a white solid (0.13 g, 34%). MS (ESI) m/z 924.1 [M+H]⁺.

### Step 2: (S)-4-(((S)-1-((S)-2-((4-(2-(2-(azidooxy)ethoxy)ethoxy)butyl)carbamoyl)pyrrolidin-1-l)-3-methyl-1-oxobutan-2-yl)amino)-3-((S)-4-methyl-2-(2-(4-(3-(o-tolyl)ureido)phenyl) acetamido)pentanamido)-4-oxobutanoic acid

To a solution of methyl (S)-4-(((S)-1-((S)-2-((4-(2-(2-(azidooxy)ethoxy)ethoxy)butyl) carbamoyl)pyrrolidin-1-yl)-3-methyl-1-oxobutan-2-yl)amino)-3-((S)-4-methyl-2-(2-(4-(3-(o-tolyl)ureido) phenyl)acetamido)pentanamido)-4-oxobutanoate (0.18 g, 0.195 mmol) in a mixture of THF (8 ml), MeOH (15 ml) and H₂O (2 ml), LiGH (12 mg, 0.49 mmol) was added and stirred at room temperature for 15 hours. The solvents were evaporated under reduced pressure. The residue was treated with HCl (1N, 5 ml). The resulting white precipitate was collected by filtration, washed with water, and air-dried to obtain the titled compound as an off-white glassy solid (0.17 g, 98%). MS (ESI) m/z 932.1 [M+Na]⁺. 1H NMR (500 MHz, DMSO-d₆) δ 12.34 (s, 1H), 9.09 (d, J=2.9 Hz, 1H), 8.36 (d, J=7.9 Hz, 1H), 8.15 (ddd, J=20.1, 15.8, 8.4 Hz, 2H), 7.98 (s, 1H), 7.82 (dd, J=10.4, 6.9 Hz, 2H), 7.52 (d, J=8.6 Hz, OH), 7.40-7.29 (m, 2H), 7.15 (q, J=6.3, 5.6 Hz, 4H), 6.93 (t, J=7.4 Hz, 1H), 4.51 (dq, J=30.1, 6.9 Hz, 1H), 4.38-4.19 (m, 3H), 3.75-3.35 (m, 19H), 3.24 (dt, J=12.3, 6.3 Hz, 1H), 3.18-3.06 (m, 1H), 2.74-2.56 (m, 1H), 2.24 (s, 3H), 2.08-1.68 (m, 3H), 1.56 (tt, J=13.2, 6.7 Hz, 1H), 1.44 (ddt, J=14.2, 10.3, 6.5 Hz, 2H), 0.92-0.73 (m, 12H).

### BA-154 was conjugated to an oligo sense strand according to general procedure type I. The product (MW: 8209.13 g/mol) was made with 99% purity and confirmed by HPLC and LCMS (m/z: 8207.66)

### Example 6: BA-161 Conjugate

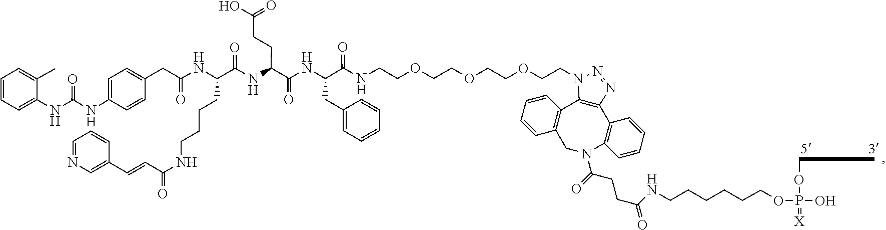

wherein X is O or S.

### BA-161: (14S,17S)-1-azido-14-benzyl-13,16-dioxo-17-((S)-6-((E)-3-(pyridin-3-yl)acrylamido)-2-(2-(4-(3-(o-tolyl)ureido)phenyl)acetamido)hexanamido)-3,6,9-trioxa-12,15-diazaicosan-20-oic acid

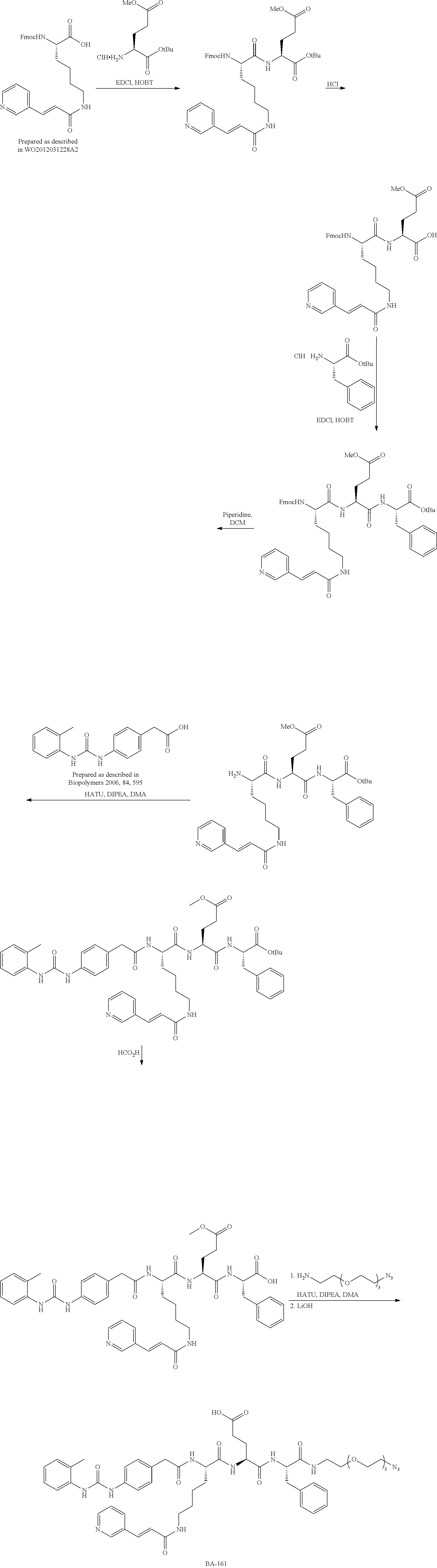

### Step 1: 1-(tert-butyl) 5-methyl N2-(((9H-fluoren-9-yl)methoxy)carbonyl)-N6-((E)-3-(pyridin-3-yl)acryloyl)-L-lysyl-L-glutamate

A solution of (E)-N2-(((9H-fluoren-9-yl)methoxy)carbonyl)-N6-(3-(pyridin-3-yl)acryloyl)-L-lysine (2.90 g, 5.805 mmol) and 1-tert-butyl 5-methyl (2S)-2-aminopentanedioate hydrochloride (1.47 g, 5.805 mmol) in DMF (29 ml) was treated with HATU (3.31 g, 8.707 mmol), followed by the addition of DIPEA (2.25 g, 17.415 mmol) dropwise at room temperature. The resulting mixture was stirred for 3 hours at room temperature under nitrogen atmosphere. The reaction was quenched with water at room temperature. The resulting mixture was extracted with EtOAc. The combined organic layers were washed with water and brine, dried over anhydrous Na₂SO₄. After filtration, the filtrate was concentrated under reduced pressure. This resulted 1-tert-butyl 5-methyl (2S)-2-[(2S)-2-{[(9H-fluoren-9-ylmethoxy)carbonyl]aminoI-6-[(2E)-3-(pyridin-3-yl)prop-2-enamido]hexanamido]pentanedioate (3.9 g, 96%) as a white solid. MS (ESI) m/z 699.9 [M+H]⁺.

### Step 2: (S)-2-((S)-2-((((9H-fluoren-9-yl)methoxy)carbonyl)amino)-6-((E)-3-(pyridin-3-yl) acrylamido)hexanamido)-5-methoxy-5-oxopentanoic acid

A solution of 1-tert-butyl 5-methyl (2S)-2-[(2S)-2-{[(9H-fluoren-9-ylmethoxy)carbonyl]amino}-6- [(2E)-3-(pyridin-3-yl)prop-2-enamido]hexanamido]pentanedioate (300 mg, 0.429 mmol) in DCM (1 ml) was treated with hydrogen chloride (3 mL) in dioxane for 3 hours at room temperature. The resulting mixture was concentrated under reduced pressure. The crude product was used in the next step directly without further purification. MS (ESI) m/z 643.6 [M+H]⁺.

### Step 3: tert-butyl (5S,8S,11S)-11-benzyl-1-(9H-fluoren-9-yl)-8-(3-methoxy-3-oxopropyl)-3,6,9-trioxo-5-(4-((E)-3-(pyridin-3-yl)acrylamido)butyl)-2-oxa-4,7,10-triazadodecan-12-oate

A solution of (2S)-2-[(2S)-2-{[(9H-fluoren-9-ylmethoxy)carbonyl]amino1-6-[(2E)-3-(pyridin-3-yl)prop-2-enamido]hexanamido]-5-methoxy-5-oxopentanoic acid (3 g, 4.67 mmol) and tert-butyl (2S)-2-amino-3-phenylpropanoate hydrochloride (2.41 g, 9.34 mmol) in DMF (30 ml) was treated with HATU (3.55 g, 9.34 mmol) at room temperature under nitrogen atmosphere followed by the addition of DIPEA (6.03 g, 46.65 mmol) dropwise at room temperature. The reaction was quenched with water/Ice at room temperature. The resulting mixture was extracted with EtOAc. The combined organic layers were washed with brine, dried over anhydrous Na₂SO₄.The residue was purified by trituration with diethyl ether. The precipitated solids were collected by filtration and washed with diethyl ether to afford the titled compound (3.8 g, 96%) as an off-white solid. MS (ESI) m/z 847.1 [M+H]⁺.

### Step 4: methyl (S)-4-((S)-2-amino-6-((E)-3-(pyridin-3-yl)acrylamido)hexanamido)-5-(((S)-1-(tert-butoxy)-1-oxo-3-phenylpropan-2-yl)amino)-5-oxopentanoate

A solution of methyl (4S)-4-{[(2S)-1-(tert-butoxy)-1-oxo-3-phenylpropan-2-yl]carbamoyl}-4-[(2S)-2-{[(9H-fluoren-9-ylmethoxy)carbonyl]amino}-6-[(2E)-3-(pyridin-3-yl)prop-2-enamido]hexanamido]butanoate (500 mg, 0.591 mmol) in DCM (20 ml) was treated with piperidine (4 ml).The resulting mixture was stirred for 2 hours at room temperature. The resulting mixture was concentrated under reduced pressure. The residue was purified by reverse flash chromatography with the following conditions: column, C18 silica gel; mobile phase, MeCN in Water, 10% to 90% gradient in 35 min; detector, UV 254 nm. The resulting product was extracted with DCM (2×150 ml). The combined organic layers were washed with brine (2×30 ml), dried over anhydrous Na₂SO₄. After filtration, the filtrate was concentrated under reduced pressure. This resulted in methyl (4S)-4-[(2S)-2-amino-6-[(2E)-3-(pyridin-3-yl)prop-2-enamido]hexanamido]-4-{[(2S)-1-(tert-butoxy)-1-oxo-3-phenylpropan-2-yl]carbamoyl}butanoate (680 mg, 44%) as an off-white solid. MS (ESI) m/z 624.6 [M+H]⁺.

### Step 5: methyl (S)-5-(((S)-1-(tert-butoxy)-1-oxo-3-phenylpropan-2-yl)amino)-5-oxo-4-((S)-6-((E)-3-(pyridin-3-yl)acrylamido)-2-(2-(4-(3-(o-tolyl)ureido)phenyl)acetamido) hexanamido) pentanoate

To a solution of methyl (S)-4-((S)-2-amino-6-((E)-3-(pyridin-3-yl)acrylamido)hexanamido)-5-(((S)-1-(tert-butoxy)-1-oxo-3-phenylpropan-2-yl)amino)-5-oxopentanoate (0.33 g, 0.523 mmol) in anhydrous DMA (3 ml), 2-(4-(3-(o-tolyl)ureido)phenyl)acetic acid (0.14 g, 0.475 mmol), HATU (0.18 g, 0.475 mmol) and DIPEA (0.083 ml, 0.475 mmol) were added. The reaction mixture was stirred at room temperature under inert atmosphere for 3 hours. Reaction mixture was diluted with water (120 ml). The resulting precipitate was filtered off, washed with water, and air-dried to obtain the titled compound as an off-white solid (0.32 g, 76%). MS (ESI) m/z 891.1 [M+H]⁺. ¹H NMR (500 MHz, DMSO-d₆) δ 8.94 (s, 1H), 8.78 (d, J=2.2 Hz, 1H), 8.58 (dd, J=4.8, 1.6 Hz, 1H), 8.25 (d, J=7.3 Hz, 1H), 8.19-8.10 (m, 2H), 8.05 (d, J=8.1 Hz, 1H), 8.00 (d, J=8.1 Hz, 1H), 7.88 (s, 1H), 7.83 (dd, J=8.2, 1.3 Hz, 1H), 7.56-7.43 (m, 2H), 7.43-7.30 (m, 2H), 7.30-7.08 (m, 9H), 6.93 (td, J=7.4, 1.3 Hz, 1H), 6.74 (d, J=15.9 Hz, 1H), 4.38-4.17 (m, 3H), 3.58 (s, 3H), 3.43 (s, 2H), 3.21-3.02 (m, 2H), 3.02-2.86 (m, 3H), 2.29 (ddd, J=12.7, 6.2, 3.1 Hz, 2H), 2.23 (s, 3H), 1.99-1.84 (m, 1H), 1.82-1.68 (m, 1H), 1.61 (s, 1H), 1.51 (dd, J=9.3, 4.9 Hz, 1H), 1.43 (s, 2H), 1.29 (s, 11H).

### Step 6: ((S)-5-methoxy-5-oxo-2-((S)-6-((E)-3-(pyridin-3-yl)acrylamido)-2-(2-(4-(3-(o-tolyl)ureido)phenyl)acetamido)hexanamido)pentanoyl)-L-phenylalanine

To a solution of methyl (S)-5-(((S)-1-(tert-butoxy)-1-oxo-3-phenylpropan-2-yl)amino)-5-oxo-4-((S)-6-((E)-3-(pyridin-3-yl)acrylamido)-2-(2-(4-(3-(o-tolyl)ureido)phenyl)acetamido) hexanamido) pentanoate (0.3 g, 0.34 mmol) in trifluoroethanol (3 ml), formic acid (0.35 ml) was added. The reaction mixture was heated at 60° C. for 15 hours. The reaction mixture was concentrated to dryness to obtain the titled compound as a yellow oil quantitatively. MS (ESI) m/z 834.7 [M+H]⁺, 856.7 [M+Na]⁺.

### Step 7: methyl (14S,17S)-1-azido-14-benzyl-13,16-dioxo-17-((S)-6-((E)-3-(pyridin-3-yl)acrylamido)-2-(2-(4-(3-(o-tolyl)ureido)phenyl)acetamido)hexanamido)-3,6,9-trioxa-12,15-diazaicosan-20-oate

To a solution of ((S)-5-methoxy-5-oxo-2-((S)-6-((E)-3-(pyridin-3-yl)acrylamido)-2-(2-(4-(3-(o-tolyl)ureido)phenyl)acetamido)hexanamido)pentanoyl)-L-phenylalanine (0.28 g, 0.34 mmol) in anhydrous DMA (3 ml), 4-(2-(2-(azidooxy)ethoxy)ethoxy)butan-1-amine (0.15 g, 0.67 mmol), HATU (0.32 g, 0.84 mmol) and DIPEA (0.24 ml, 1.34 mmol) were added. The reaction mixture was stirred at room temperature under inert atmosphere for 18 hours. Reaction mixture was diluted with water (100 ml) and extracted with EtOAc (3×25 ml). The combined organic extracts were washed with brine (15 ml), dried over anhydrous Na₂SO₄ and concentrated under reduced pressure to obtain the titled compound as a beige solid (0.25 g, 71%). MS (ESI) m/z 1035.2 [M+H]⁺.

### Step 8: (14S,17S)-1-azido-14-benzyl-13,16-dioxo-17-((S)-6-((E)-3-(pyridin-3-yl)acrylamido)-2-(2-(4-(3-(o-tolyl)ureido)phenyl)acetamido)hexanamido)-3,6,9-trioxa-12,15-diazaicosan-20-oic acid

To a solution of methyl (14S,17S)-1-azido-14-benzyl-13,16-dioxo-17-((S)-6-((E)-3-(pyridin-3-yl)acrylamido)-2-(2-(4-(3-(o-tolyl)ureido)phenyl)acetamido)hexanamido)-3,6,9-trioxa-12,15-diazaicosan-20-oate (0.24 g, 0.23 mmol) in a methanol/water/THF (6 ml, 1:1:1) mixture, LiGH (17 mg, 0.70 mmol) was added and stirred at room temperature for 5 hours. The solvents were evaporated under reduced pressure. The residue was treated with 10% citric acid solution and purified by reverse-phase column chromatography (C18) using a gradient 0-40% ACN in water (+0.1% formic acid) to afford the titled compound as a white spongy solid (0.13 g, 55%). MS (ESI) m/z 1021.2 [M+H]⁺, 1043.1 [M+Na]⁺. ¹H NMR (500 MHz, DMSO-d₆) δ 9.15 (s, 1H), 8.74 (d, J=2.2 Hz, 1H), 8.53 (dd, J=4.7, 1.6 Hz, 1H), 8.26 (s, 4H), 8.23-8.14 (m, 2H), 8.14-7.92 (m, 1H), 7.89 (d, J=8.2 Hz, 1H), 7.81 (d, J=8.2 Hz, 1H), 7.50-7.32 (m, 4H), 7.27-7.08 (m, 8H), 6.93 (td, J=7.5, 1.4 Hz, 1H), 6.73 (d, J=15.9 Hz, 1H), 4.45 (td, J=8.3, 5.3 Hz, 1H), 4.19 (dq, J=13.1, 7.8 Hz, 2H), 3.63-3.29 (m, 18H), 3.21 (dt, J=11.7, 6.3 Hz, 2H), 3.17-3.07 (m, 2H), 3.02-2.90 (m, 1H), 2.23 (s, 3H), 2.14 (tt, J=16.6, 8.5 Hz, 2H), 1.83 (dt, J=15.7, 6.9 Hz, 1H), 1.67 (ddd, J=31.5, 16.3, 8.3 Hz, 2H), 1.43 (s, 2H), 1.36-1.13 (m, 2H).

BA-161 was conjugated to an oligo sense strand according to general procedure type I. The product (MW: 8367.17 g/mol) was made with 98% purity and confirmed by HPLC and LCMS (m/z: 8365.37).

### Example 7: BA-171 Conjugates

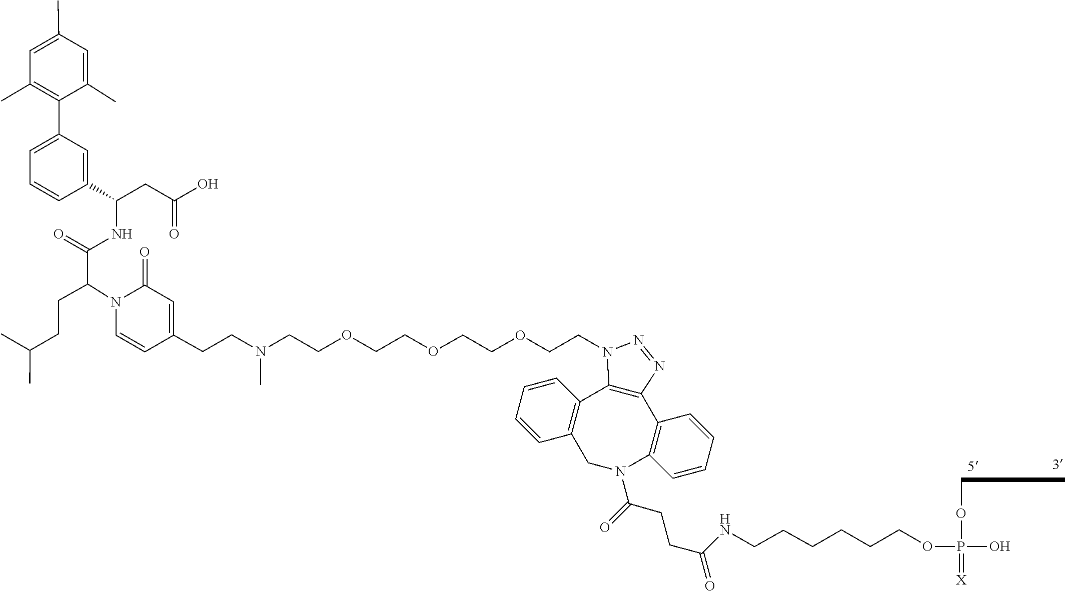

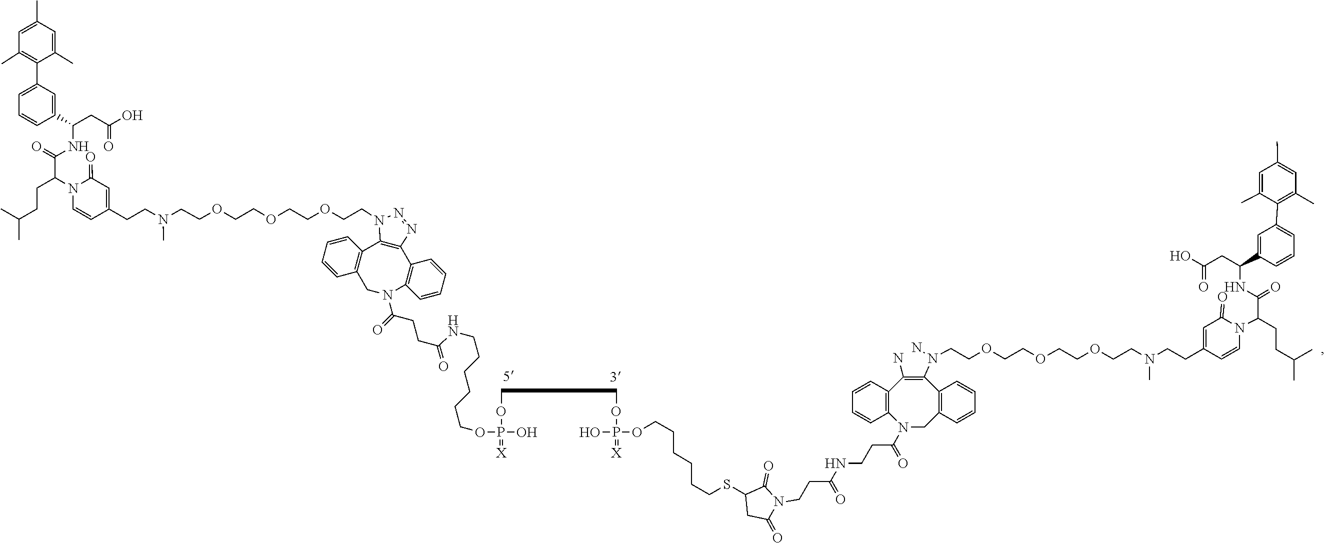

wherein X is O or S.

### BA-171: (3S)-3-(2-(4-(14-azido-3-methyl-6,9,12-trioxa-3-azatetradecyl)-2-oxopyridin-1(2H)-yl)-5-methylhexanamido)-3-(2′,4′,6′-trimethyl-[1,1′-biphenyl]-3-yl)propanoic acid

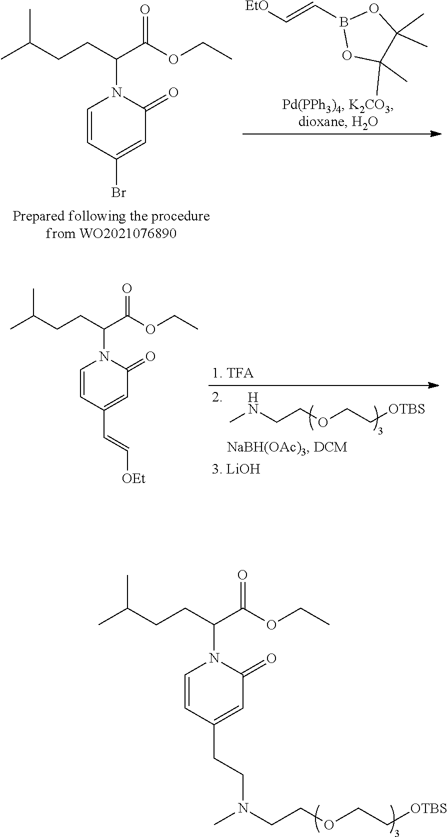

### Step 1: ethyl (E)-2-(4-(2-ethoxyvinyl)-2-oxopyridin-1(2H)-yl)-5-methylhexanoate

To a stirred solution of ethyl 2-(4-bromo-2-oxopyridin-1-yl)-5-methylhexanoate (4.8 g, 14.54 mmol) and 2-[(E)-2-ethoxyethenyl]-4,4,5,5-tetramethyl-1,3,2-dioxaborolane (5.76 g, 29.081 mmol) in dioxane (50 ml) and H₂O (5 ml) were added K₂CO₃ (4.02 g, 29.10 mmol) and Pd(PPh₃)₄(1.68 g, 1.45 mmol) at room temperature under nitrogen atmosphere. The resulting mixture was stirred for overnight at 70° C. under nitrogen atmosphere. The mixture was allowed to cool down to room temperature. The reaction was quenched by the addition of water at room temperature. The resulting mixture was extracted with EtOAc. The combined organic layers were washed with brine, dried over anhydrous Na₂SO₄. After filtration, the filtrate was concentrated under reduced pressure. The residue was purified by silica gel column chromatography, eluted with PE/EA (1:1) to afford product (Pinacol containing). The residue was further purified by reversed-phase flash chromatography with the following conditions: column, C18 silica gel; mobile phase, MeCN in Water, 10-100% gradient in 25 min; detector, UV 254 nm. This resulted in ethyl 2-(4-[(E)-2-ethoxyethenyl]-2-oxopyridin-1-yl-5-methylhexanoate (4 g, 86%) as a brown oil. MS (ESI) m/z 322.1 [M+H]⁺. ¹H NMR (300 MHz, CDCl₃) δ 7.16 (m, J=19.4, 10.1 Hz, 2H), 6.37 (s, 1H), 6.19 (d, J=7.4 Hz, 1H), 5.60 (d, J=13.0 Hz, 1H), 5.54 (m, J=10.2, 5.6 Hz, 1H), 4.19 (q, J=7.2 Hz, 2H), 3.94 (q, J=7.1 Hz, 2H), 2.29-2.03 (m, 1H), 1.86 (d, J=12.3 Hz, 1H), 1.57 (m, J=13.3, 6.8 Hz, 1H), 1.35 (t, J=7.0 Hz, 3H), 1.24 (m, J=8.5, 7.8 Hz, 4H), 1.14-1.04 (m, 1H), 0.87 (m, J=6.8, 3.0 Hz, 6H).

### Step 2: ethyl 5-methyl-2-(2-oxo-4-(2-oxoethyl)pyridin-1(2H)-yl)hexanoate

Into a 250 mL round-bottom flask were added ethyl 2-(4-[(E)-2-ethoxyethenyl]-2-oxopyridin-1-yl-5-methylhexanoate (3.9 g, 12.134 mmol) and TFA (40 ml) at room temperature. The resulting mixture was stirred for overnight at room temperature. The resulting mixture was concentrated under reduced pressure. The residue was purified by silica gel column chromatography, eluted with PE/EA (1:2) to afford ethyl 5-methyl-2-[2-oxo-4-(2-oxoethyl)pyridin-1-yl]hexanoate (1.9 g, 53%) as a yellow oil. MS (ESI) m/z 294.2. ¹H NMR (300 MHz, Chloroform-d) δ 9.76 (t, J=2.0 Hz, 1H), 7.35 (d, J=7.1 Hz, 1H), 6.54-6.47 (m, 1H), 6.14 (m, J=7.2, 2.0 Hz, 1H), 5.56 (m, J=10.1, 5.6 Hz, 1H), 4.21 (q, J=7.1 Hz, 2H), 3.56 (d, J=2.0, 0.8 Hz, 2H), 2.19 (m, J=14.0, 11.1, 5.5 Hz, 1H), 1.88 (m, J=18.8, 10.4, 4.8 Hz, 1H), 1.59 (m, J=13.3, 6.7 Hz, 1H), 1.34-1.16 (m, 4H), 1.16-0.98 (m, 1H), 0.88 (q, J=6.6, 3.1 Hz, 6H).

### Step 3: ethyl 5-methyl-2-(2-oxo-4-(2,2,3,3,16-pentamethyl-4,7,10,13-tetraoxa-16-aza-3-silaoctadecan-i8-yl)pyridin-1(2H)-yl)hexanoate

To a stirred solution of ethyl 5-methyl-2-[2-oxo-4-(2-oxoethyl)pyridin-1-yl]hexanoate (1.8 g, 6.136 mmol) in DCM (27 ml) was added 15,15,16,16-tetramethyl-5,8,11,14-tetraoxa-2-aza-15-silaheptadecane (1.97 g, 6.127 mmol), STAB (2.60 g, 12.268 mmol) at room temperature. The resulting mixture was stirred for 2 hours at room temperature. The reaction was quenched by the addition of water (100 ml) at room temperature. The resulting mixture was extracted with DCM (3×120 ml). The combined organic layers were washed with brine (2×50 ml), dried over anhydrous Na₂SO₄. After filtration, the filtrate was concentrated under reduced pressure. The residue was purified by silica gel column chromatography, eluted with DCM/MeOH (10:1) to afford ethyl 5-methyl-2-[2-oxo-4-(2,2,3,3,16-pentamethyl-4,7,10,13-tetraoxa-16-aza-3-silaoctadecan-18-yl)pyridin-1-yl]hexanoate (1.6 g, 44%) as a brown yellow oil. MS(ESI) m/z 599.3 [M+H]⁺. ¹HNMR (300 MHz, Acetonitrile-d3) 6 7.25 (d, J=7.1 Hz, 1H), 6.20 (s, 1H), 6.11 (m, J=7.1, 2.0 Hz, 1H), 5.08 (m, J=10.2, 5.4 Hz, 1H), 4.08 (q, J=7.1 Hz, 2H), 3.66 (m, J=5.7, 4.4 Hz, 2H), 3.57-3.47 (m, 11H), 3.47-3.37 (m, 3H), 2.68-2.48 (m, 4H), 2.24 (s, 3H), 2.15-1.99 (m, 1H), 1.90 (m, 1H), 1.48 (m, J=13.4, 6.8 Hz, 1H), 1.25-1.11 (m, 5H), 1.07-0.88 (m, 1H), 0.83 (s, 9H), 0.79 (m, J=6.6, 3.0 Hz, 6H), 0.02 (s, 6H).

### Step 4: 5-methyl-2-(2-oxo-4-(2,2,3,3,16-pentamethyl-4,7,10,13-tetraoxa-16-aza-3-silaoctadecan-18-yl)pyridin-1(2H)-yl)hexanoic acid

To a stirred solution of ethyl 5-methyl-2-[2-oxo-4-(2,2,3,3,16-pentamethyl-4,7,10,13-tetraoxa-16-aza-3-silaoctadecan-18-yl)pyridin-1-yl]hexanoate (1.4 g, 2.338 mmol) in THF (14 ml) and H₂O (2.8 ml) were added LiOH (223.95 mg, 9.352 mmol) at room temperature. The resulting mixture was stirred for 2 hours at room temperature. To the above mixture was added AcOH (0.71 g, 11.688 mmol) in H₂O (14 ml) dropwise at 0° C. The resulting mixture was extracted with CH₃C₁ (3×100 ml). The combined organic layers were washed with brine (2×40 ml), dried over anhydrous MgSO₄. After filtration, the filtrate was concentrated under reduced pressure. The crude product was used in the next step directly without further purification. This resulted in 5-methyl-2-[2-oxo-4-(2,2,3,3,16-pentamethyl-4,7,10,13-tetraoxa-16-aza-3-silaoctadecan-18-yl)pyridin-1-yl]hexanoic acid (0.90 g, 67%) as a yellow semi-solid. MS(ESI) m/z 571.3 [M+H]⁺.

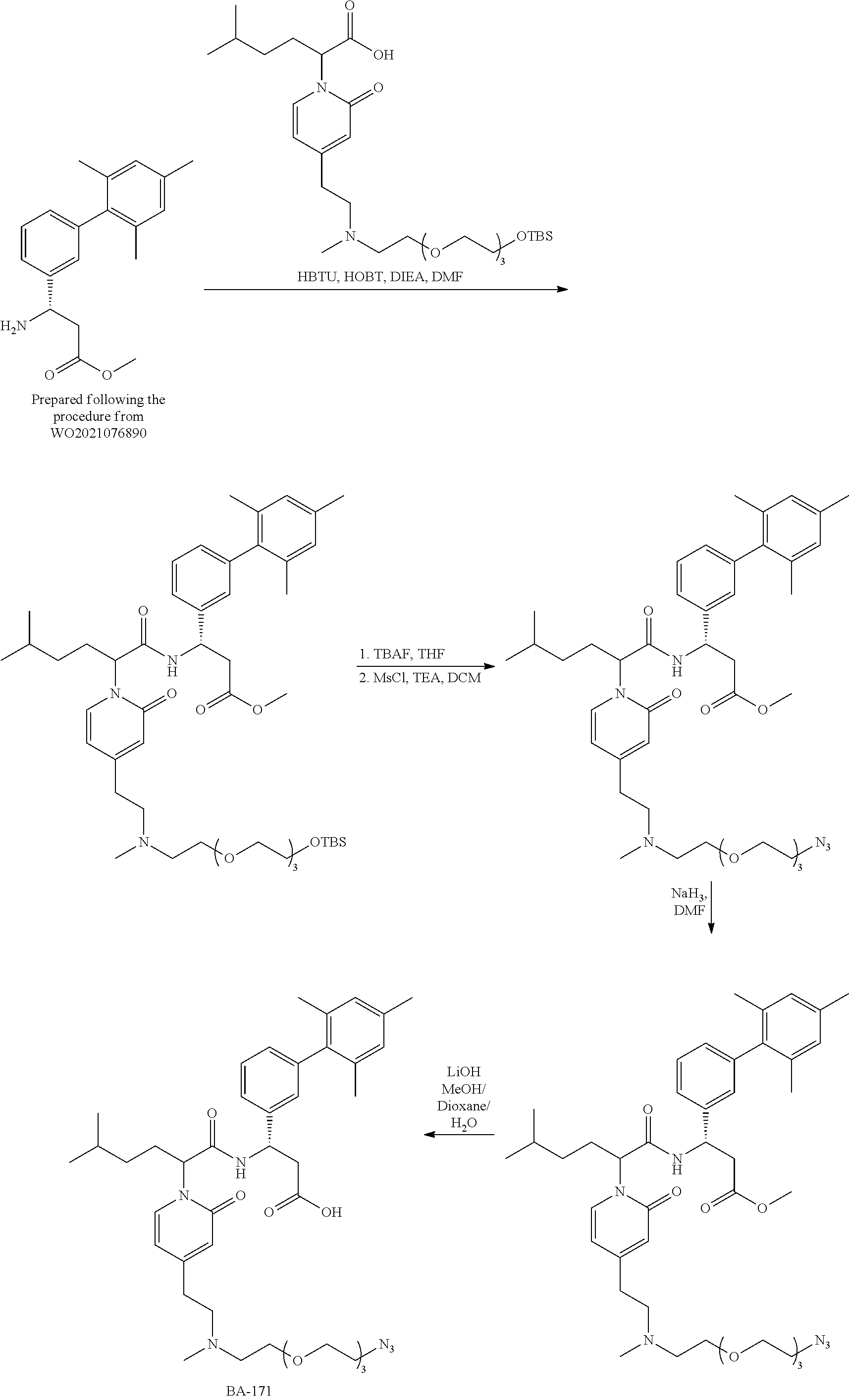

### Step 5: methyl (3S)-3-(5-methyl-2-(2-oxo-4-(2,2,3,3,16-pentamethyl-4,7,10,13-tetraoxa-16-aza-3-silaoctadecan-18-yl)pyridin-1(2H)-yl)hexanamido)-3-(2′,4′,6′-trimethyl-[1,1′-biphenyl]-3-yl)propanoate

To a stirred mixture of methyl (3S)-3-amino-3-(2′,4′,6′-trimethyl-[1,1′-biphenyl]-3-ylpropanoate (600 mg, 2.017 mmol) and 5-methyl-2-[2-oxo-4-(2,2,3,3,16-pentamethyl-4,7,10,13-tetraoxa-16-aza-3-silaoctadecan-18-yl)pyridin-1-yl]hexanoic acid (1.15 g, 2.017 mmol) in DMF (12 ml) were added DIEA (782.27 mg, 6.051 mmol), HBTU (765.13 mg, 2.017 mmol) and HOBT (27.26 mg, 0.202 mmol) at room temperature under nitrogen atmosphere. The resulting mixture was stirred for 2 h at room temperature under nitrogen atmosphere. The residue was purified by reversed-phase flash chromatography with the following conditions: column, C18 silica gel; mobile phase, MeCN in Water, 40-100% gradient in 10 min; 100% gradient in 20 min; detector, UV 220 nm/305 nm. This resulted in methyl (3S)-3-(5-methyl-2-[2-oxo-4-(2,2,3,3,16-pentamethyl-4,7,10,13-tetraoxa-16-aza-3-silaoctadecan-18-yl)pyridin-1-yl]hexanamido-3-(2′,4′,6′-trimethyl-[1,1′-biphenyl]-3-ylpropanoate (509.0 mg, 29%) as a brown oil. MS(ESI) m/z 850.6 [M+H]⁺. ¹H NMR (300 MHz, DMSO-d₆) δ 8.95 (m, J=27.0, 8.5, 5.6 Hz, 1H), 7.57 (m, J=12.3, 7.3, 5.2 Hz, 1H), 7.45-7.19 (m, 2H), 7.12-6.86 (m, 4H), 6.18 (m, J=21.3, 11.5, 5.6 Hz, 2H), 5.77 (d, J=5.1 Hz, 1H), 5.34 (m, J=61.7, 13.7, 5.9 Hz, 2H), 3.68 (q, J=5.2 Hz, 2H), 3.63-3.39 (m, 15H), 3.32 (s, 2H), 2.83 (t, J=6.5 Hz, 2H), 2.24 (m, J=12.7, 5.5 Hz, 6H), 1.95-1.80 (m, 7H), 1.70-1.57 (m, 1H), 1.57-1.47 (m, 1H), 1.47-1.34 (m, 1H), 1.24 (d, J=5.5 Hz, 2H), 1.15-0.96 (m, 1H), 0.93-0.79 (m, 13H), 0.72 (t, J=6.0 Hz, 4H), 0.08-0.01 (m, 6H).

### Step 6: methyl (3S)-3-(2-(4-(14-hydroxy-3-methyl-6,9,12-trioxa-3-azatetradecyl)-2-oxopyridin-1(2H)-yl)-5-methylhexanamido)-3-(2′,4′,6′-trimethyl-[1,1′-biphenyll-3-yl)propanoate

To a solution of methyl (3S)-3-(5-methyl-2-(2-oxo-4-(2,2,3,3,16-pentamethyl-4,7,10,13-tetraoxa-16-aza-3-silaoctadecan-18-yl)pyridin-1(2H)-yl)hexanamido)-3-(2′,4′,6′-trimethyl-[1,1′-biphenyl]-3-yl)propanoate (0.25 g, 0.294 mmol) in THF (2 ml), TBAF (0.35 ml, 0.35 mmol, 1M in THF) was added dropwise. The reaction mixture was stirred at room temperature under inert atmosphere for 2 hours. Reaction mixture was diluted with saturated ammonium chloride solution (15 ml) and extracted with EtOAc (3×15 ml). The combined organic extracts were washed with brine (10 mL), dried over anhydrous Na₂SO₄ and concentrated under reduced pressure. The crude residue was purified by column chromatography using 0-15% MeOH in DCM to obtain the titled product as a clear oil (0.12 mg, 55%). MS (ESI) m/z 759.1 [M+Na]⁺.

### Step 7: methyl (3S)-3-(5-methyl-2-(4-(3-methyl-i4-((methylsulfonyl)oxy)-6,9,12-trioxa-3-azatetradecyl)-2-oxopyridin-1(2H)-yl)hexanamido)-3-(2′,4′,6′-trimethyl-[1,1′-biphenyll-3-yl)propanoate

To a solution of methyl (3S)-3-(2-(4-(14-hydroxy-3-methyl-6,9,12-trioxa-3-azatetradecyl)-2-oxopyridin-1(2H)-yl)-5-methylhexanamido)-3-(2′,4′,6′-trimethyl-[1,1′-biphenyl]-3-yl)propanoate (0.12 g, 0.163 mmol) in DCM (1 ml), methanesulfonyl chloride (0.016 ml, 0.212 mmol) and Et₃N (0.045 ml, 0.33 mmol) were added. The reaction mixture was stirred at 0° C. for 2 hours. Reaction mixture was diluted with saturated aqueous sodium bicarbonate (10 ml) and extracted with DCM (3×20 ml). The combined organic extracts were washed with brine (10 ml), dried over anhydrous Na₂SO₄ and concentrated under reduced pressure to obtain titled product as a brown oil (0.11 g, 83%). MS (ESI) m/z 815.1 [M+H]⁺.

### Step 8: methyl (3S)-3-(2-(4-(14-azido-3-methyl-6,9,12-trioxa-3-azatetradecyl)-2-oxopyridin-1(2H)-yl)-5-methylhexanamido)-3-(2′,4′,6′-trimethyl-[1,1′-biphenyll-3-yl)propanoate

To a solution of methyl (3S)-3-(5-methyl-2-(4-(3-methyl-14-((methylsulfonyl)oxy)-6,9,12-trioxa-3-azatetradecyl)-2-oxopyridin-1(2H)-yl)hexanamido)-3-(2′,4′,6′-trimethyl-[1,1′-biphenyl]-3-yl)propanoate (0.11 g, 0.135 mmol) in DMF (1 ml), sodium azide (24 mg, 0.37 mmol) was added. The reaction mixture was heated at 65° C. for 2 hours. Reaction mixture was diluted with water (15 ml) and extracted with EtOAc (3×15 ml). The combined organic extracts were washed with brine (10 ml), dried over anhydrous Na₂SO₄ and concentrated under reduced pressure. The crude material was purified by silica gel column chromatography using a gradient 0-15% MeOH in DCM to obtain the titled compound as a clear oil (45 mg, 44%). MS (ESI) m/z 762.2 [M+H]⁺.

### Step 9: (3S)-3-(2-(4-(14-azido-3-methyl-6,9,12-trioxa-3-azatetradecyl)-2-oxopyridin-1(2H)-yl)-5-methylhexanamido)-3-(2′,4′,6′-trimethyl-[1,1′-biphenyll-3-yl)propanoic acid

To a solution of methyl (3S)-3-(2-(4-(14-azido-3-methyl-6,9,12-trioxa-3-azatetradecyl)-2-oxopyridin-1(2H)-yl)-5-methylhexanamido)-3-(2′,4′,6′-trimethyl-[1,1′-biphenyl]-3-yl)propanoate (45 mg, 0.059 mmol) in a mixture of methanol/water/dioxane (1.5 ml, 1:1:1), LiGH (4 mg, 0.177 mmol) was added and stirred at room temperature for 2 hours. The solvents were evaporated in vacuo. The residue was treated with 10% citric acid solution and extracted with EtOAc (3×15 ml). The combined organic extracts were washed with brine (5 ml), dried over anhydrous Na₂SO₄ and concentrated under reduced pressure to obtain the titled compound as a clear oil (30 mg, 75%). MS (ESI) m/z 748.1 [M+H]⁺. ¹H NMR (500 MHz, DMSO-d₆) δ 12.35 (bs, 1H), 9.00 (d, J=10 Hz, 1H), 7.69 (d, J=10 Hz, 1H), 7.37 (t, J=10 Hz, 1H), 7.27 (d, J=5 Hz, 1H), 7.06 (s, 1H), 6.99 (d, J=5 Hz, 1H), 6.91 (s, 2H), 6.29 (s, 1H), 6.18 (d, J=5 Hz, 1H), 5.47 (q, J=5 Hz, 1H), 5.17 (q, J=5 Hz, 1H), 4.09 (m, 3H), 2.64 (t, 5 Hz, 2H), 3.61-3.57 (m, 13H), 3.16 (s, 6H), 2.26 (s, 3H), 1.90 (s, 6H), 1.87-1.79 (m, 1H), 1.68-1.64 (m, 1H), 1.41-1.35 (m, 1H), 0.95-0.82 (m, 3H), 6.60 (d, J=10 Hz, 6H).

BA-171 was conjugated to an oligo sense strand according to general procedure type I. The product (MW: 8458.36 g/mol) was made with 98% purity and confirmed by HPLC and LCMS (m/z: 8456.9).

BA-171 was bis-conjugated to an oligo sense strand according to general procedure type IIA. The product (MW: 9666.87 g/mol) was made with 89% purity and confirmed with HPLC and LCMS (m/z: 9664.94).

### Example 8: BA-172 Conjugate

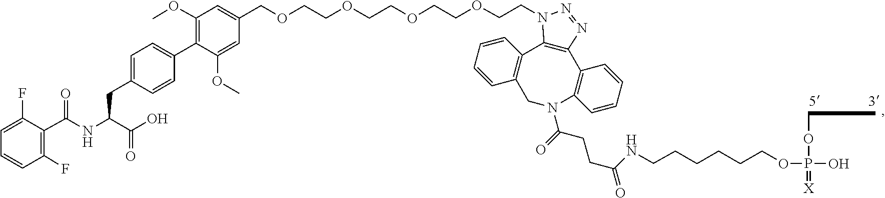

wherein X is O or S.

BA-172: (S)-3-(4′-(13-azido-2,5,8,11-tetraoxatridecyl)-2′,6′-dimethoxy-[1,1′-biphenyl]-4-yl)-2-(2,6-difluorobenzamido)propanoic acid

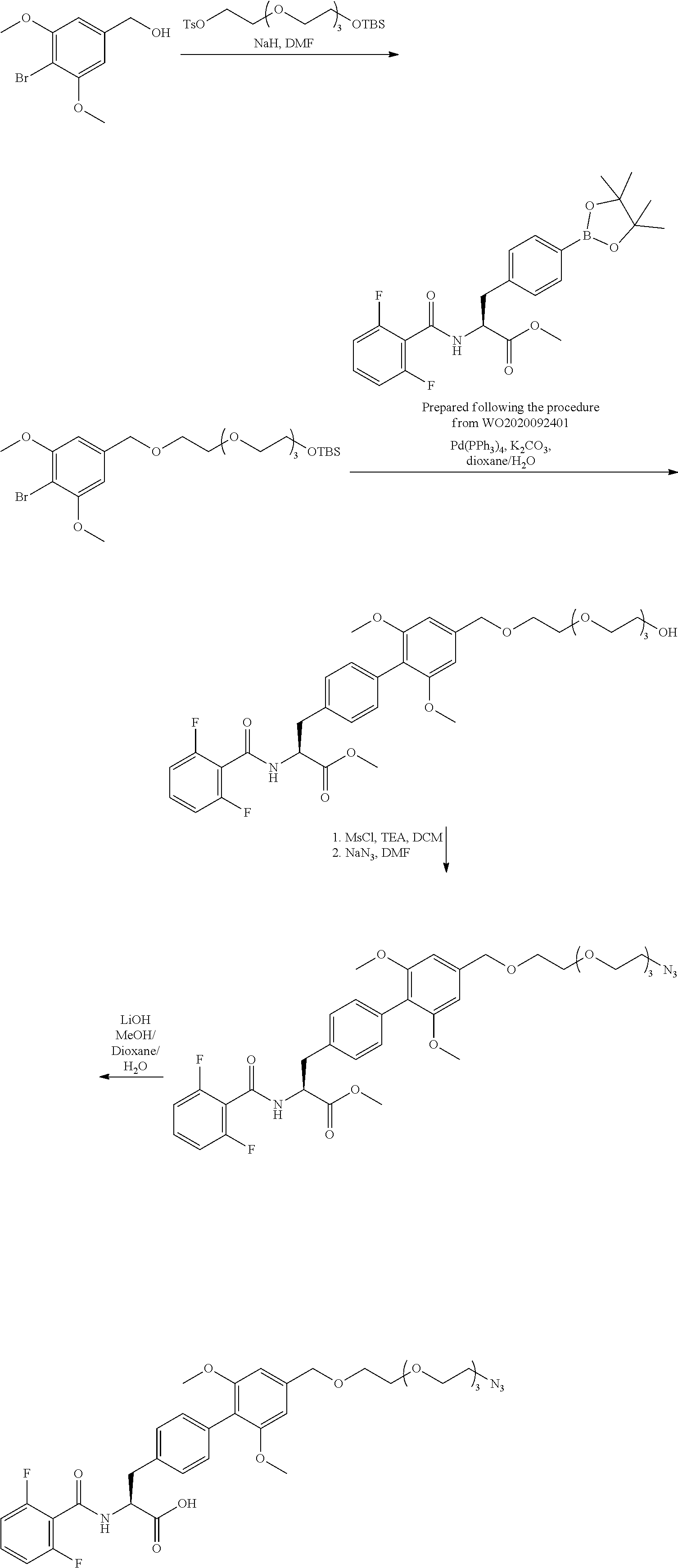

### Step 1: 1-(4-bromo-3,5-dimethoxyphenyl)-15,15,16,16-tetramethyl-2,5,8,11,14-pentaoxa-15-silaheptadecane

To a stirred solution of (4-bromo-3,5-dimethoxyphenyl)methanol (1 g, 4.05 mmol) in DMF (10.00 ml) was treated with NaH (0.24 g, 6.070 mmol, 60%) dropwise at 0° C. at N2 atmosphere. The resulting mixture was stirred for 5 min at 0° C. at N2 atmosphere. To the above mixture was added 2,2,3,3-tetramethyl-4,7,10,13-tetraoxa-3-silapentadecan-15-yl 4-methylbenzenesulfonate (2.25 g, 4.856 mmol) in portions at 0° C. The resulting solution was allowed to react at room temperature for overnight at N2 atmosphere. The reaction was quenched with water at room temperature. The reaction was quenched by the addition of water (100 ml) at room temperature. The resulting mixture was extracted with EA (3×100 ml). The combined organic layers were washed with water (3×100 ml) and saturated NaCl solution (100 ml), dried over Na₂SO₄. After filtration, the filtrate was concentrated under reduced pressure. The residue was purified by silica gel column chromatography, eluted with PE/EA to afford 1-(4-bromo-3,5-dimethoxyphenyl)-2,5,8,11-tetraoxatridecan-13-ol (800 mg, 47%) as a yellow oil. MS(ESI) m/z 537.2 [M+Na]⁺.

### Step 2: methyl (S)-2-(2,6-difluorobenzamido)-3-(2′,6′-dimethoxy-4′-(15,15,16,16-tetramethyl-2,5,8,11,14-pentaoxa-15-silaheptadecyl)-[1,1′-biphenyll-4-yl)propanoate

A solution of 1-(4-bromo-3,5-dimethoxyphenyl)-2,5,8,11-tetraoxatridecan-13-ol (670 mg, 1.583 mmol) and methyl (2S)-2-[(2,6-difluorophenyl)formamido]-3-[4-(4,4,5,5-tetramethyl-1,3,2-dioxaborolan-2-yl)phenyl]propanoate (1.06 g, 2.374 mmol) in 1,4-dioxane(10 ml) was treated with Pd(PPh₃)₄(182.91 mg, 0.158 mmol) and K₂CO₃ (656.25 mg, 4.749 mmol) and H₂O (2.3 ml, 127.671 mmol) dropwise at rt at N2 atmosphere. The reaction under nitrogen protection at 90° C. for 5 hours. The reaction was quenched with water at room temperature. The aqueous layer was extracted with EtOAc (3×100 ml). The resulting mixture was concentrated under vacuum. The residue was purified by reversed-phase flash chromatography with the following conditions: column, C18 silica gel; mobile phase, MeCN in Water (0.1% FA), 10-50% gradient in 10 min; detector, UV 254 nm. This resulted in methyl (2S)-2-[(2,6-difluorophenyl)formamido]-3-[4′-(13-hydroxy-2,5,8,11-tetraoxatridecan-1-yl)-2′,6′-dimethoxy-[1,1′-biphenyl]-4-yl]propanoate (440 mg, 42.01%) as a yellow oil. MS(ESI) m/z 662.3 [M+H]⁺. 1H NMR (300 MHz, DMSO-d₆) δ 9.33 (d, J=7.6 Hz, 1H), 7.87-7.29 (m, 3H), 7.26-7.09 (m, 6H), 6.70 (s, 2H), 4.67 (td, J=8.6, 5.3 Hz, 1H), 4.58 (t, J=5.4 Hz, 1H), 4.52 (s, 2H), 3.66 (d, J=6.1 Hz, 9H), 3.60 (s, 4H), 3.44 (dt, J=20.2, 5.3 Hz, 8H), 3.43 (d, J=52.7 Hz, 2H), 3.08-2.98 (m, 2H), 2.98 (s, 2H).

### Step 3: methyl (S)-2-(2,6-difluorobenzamido)-3-(2′,6′-dimethoxy-4′-(13-((methylsulfonyl)oxy)-2,5,8,11-tetraoxatridecyl)-[1,1′-biphenyll-4-yl)propanoate

To a solution of methyl (S)-2-(2,6-difluorobenzamido)-3-(4′-(13-hydroxy-2,5,8,11-tetraoxatridecyl)-2′,6′-dimethoxy-[1,1′-biphenyl]-4-yl)propanoate (0.17 g, 0.257 mmol) in DCM (1.5 ml), methanesulfonyl chloride (0.023 ml, 0.334 mmol) and Et₃N (0.063 ml, 0.45 mmol) were added. The reaction mixture was stirred at 0° C. for 2 hours. Reaction mixture was diluted with saturated aqueous sodium bicarbonate (10 ml) and extracted with DCM (3×20 ml). The combined organic extracts were washed with brine (10 ml), dried over anhydrous Na₂SO₄ and concentrated under reduced pressure to obtain titled product as a brown oil (0.18 g, 95%). MS (ESI) m/z 740.8 [M+H]⁺.

### Step 4: methyl (S)-3-(4′-(13-azido-2,5,8,11-tetraoxatridecyl)-2′,6′-dimethoxy-[1,1′-biphenyll-4-yl)-2-(2,6-difluorobenzamido)propanoate

To a solution of methyl (S)-2-(2,6-difluorobenzamido)-3-(2′,6′-dimethoxy-4′-(13-((methylsulfonyl)oxy)-2,5,8,11-tetraoxatridecyl)-[1,1′-biphenyl]-4-yl)propanoate (0.18 g, 0.243 mmol) in DMF (1.5 ml), sodium azide (24 mg, 0.37 mmol) was added. The reaction mixture was heated at 65° C. for 2 hours. Reaction mixture was diluted with water (20 ml) and extracted with EtOAc (3×15 ml). The combined organic extracts were washed with brine (10 ml), dried over anhydrous Na₂SO₄ and concentrated under reduced pressure. The crude material was purified by silica gel column chromatography using a gradient 20-80% EtOAc in hexane to obtain the titled compound as a clear oil (0.11 g, 65%). MS (ESI) m/z 687.8 [M+H]⁺.

### Step 5: (S)-3-(4′-(13-azido-2,5,8,11-tetraoxatridecyl)-2′,6′-dimethoxy-[1,1′-biphenyll-4-yl)-2-(2,6-difluorobenzamido)propanoic acid

To a solution of methyl (S)-3-(4′-(13-azido-2,5,8,11-tetraoxatridecyl)-2′,6′-dimethoxy-[1,1-biphenyl]-4-yl)-2-(2,6-difluorobenzamido)propanoate (0.10 g, 0.146 mmol) in a mixture of methanol/water/dioxane (1.5 ml, 1:1:1), LiGH (11 mg, 0.44 mmol) was added and stirred at room temperature for 5 hours. The solvents were evaporated under reduced pressure. The residue was treated with 10% citric acid solution and extracted with EtOAc (3×15 ml). The combined organic extracts were washed with brine (5 ml), dried over anhydrous Na₂SO₄ and concentrated to obtain the titled compound as a clear oil (80 mg, 83%). MS (ESI) m/z 695.7 [M+Na]⁺. ¹H NMR (500 MHz, DMSO-d₆) δ 9.15 (d, J=10 Hz, 1H), 7.50 (quint, J=5 Hz, 1H), 7.25 (d, J=10 Hz, 2H), 7.16-7.10 (m, 4H), 6.69 (s, 2H), 4.63-4.59 (m, 1H), 4.52 (s, 2H), 4.03 (q, J=5 Hz, 1H), 3.65 (s, 6H), 3.60 (s, 6H), 3.55-3.51 (m, 4H), 3.38-3.33 (m, 3H), 3.15 (dd, , J=15, 5 Hz, 1H), 2.99 (dd, J=15, 5 Hz, 1H), 1.17 (t, J=10 Hz, 2H).

BA-172 was conjugated to an oligo sense strand according to general procedure type I. The product (MW: 8384.09 g/mol) was made with 98% purity and confirmed by HPLC and LCMS (m/z: 8382.58).

### Example 9: BA-202 Conjugates

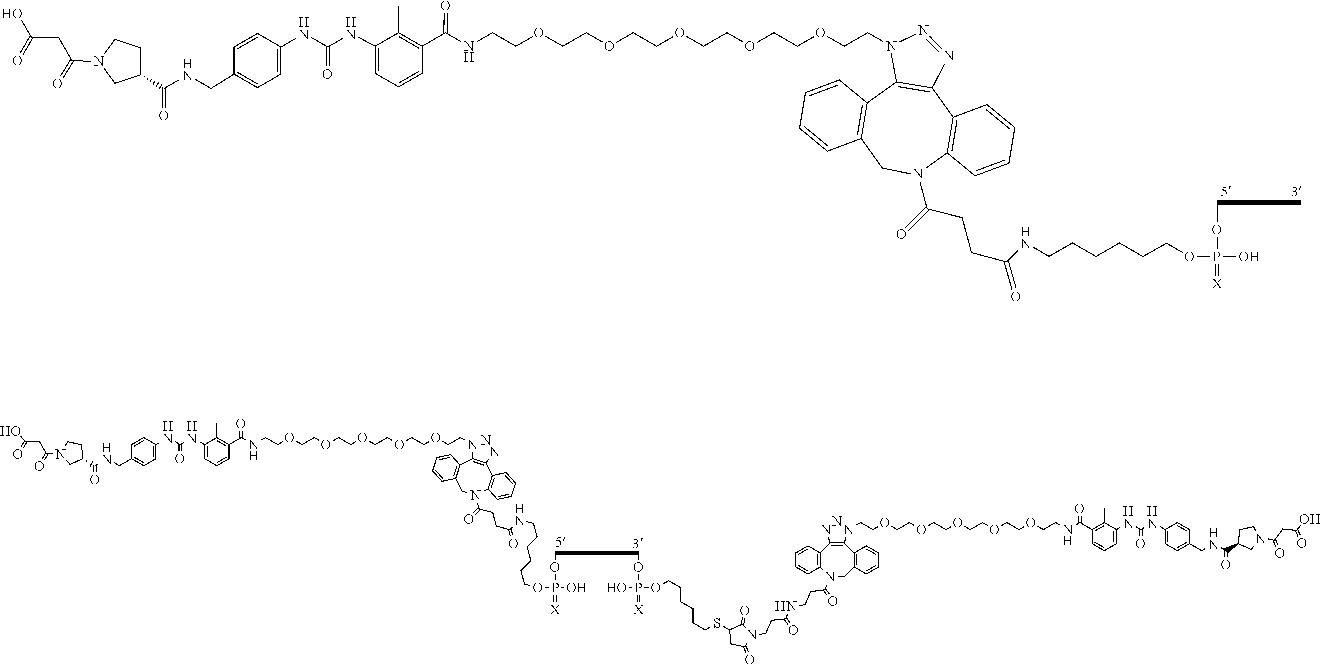

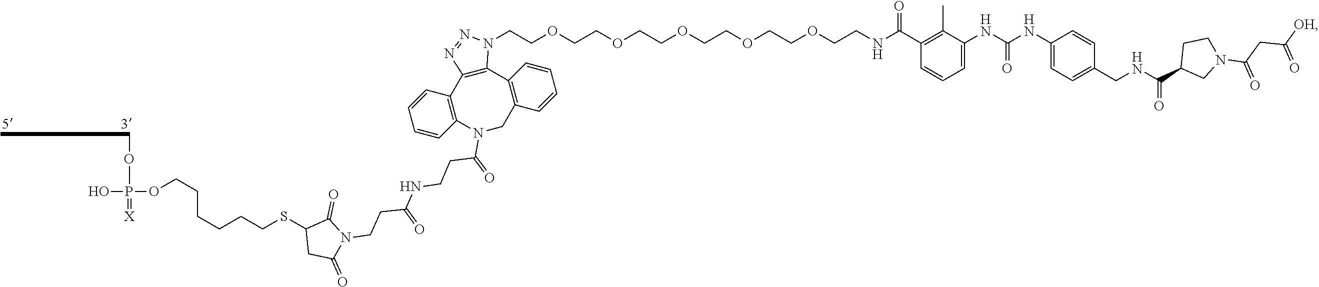

wherein X is O or S. BA-202: (S)-3-(3-((4-(3-(3-((17-azido-3,6,9,12,15-pentaoxaheptadecyl)carbamoyl)-2-methylphenyl) ureido) benzyl)carbamoyl)pyrrolidin-1-yl)-3-oxopropanoic acid

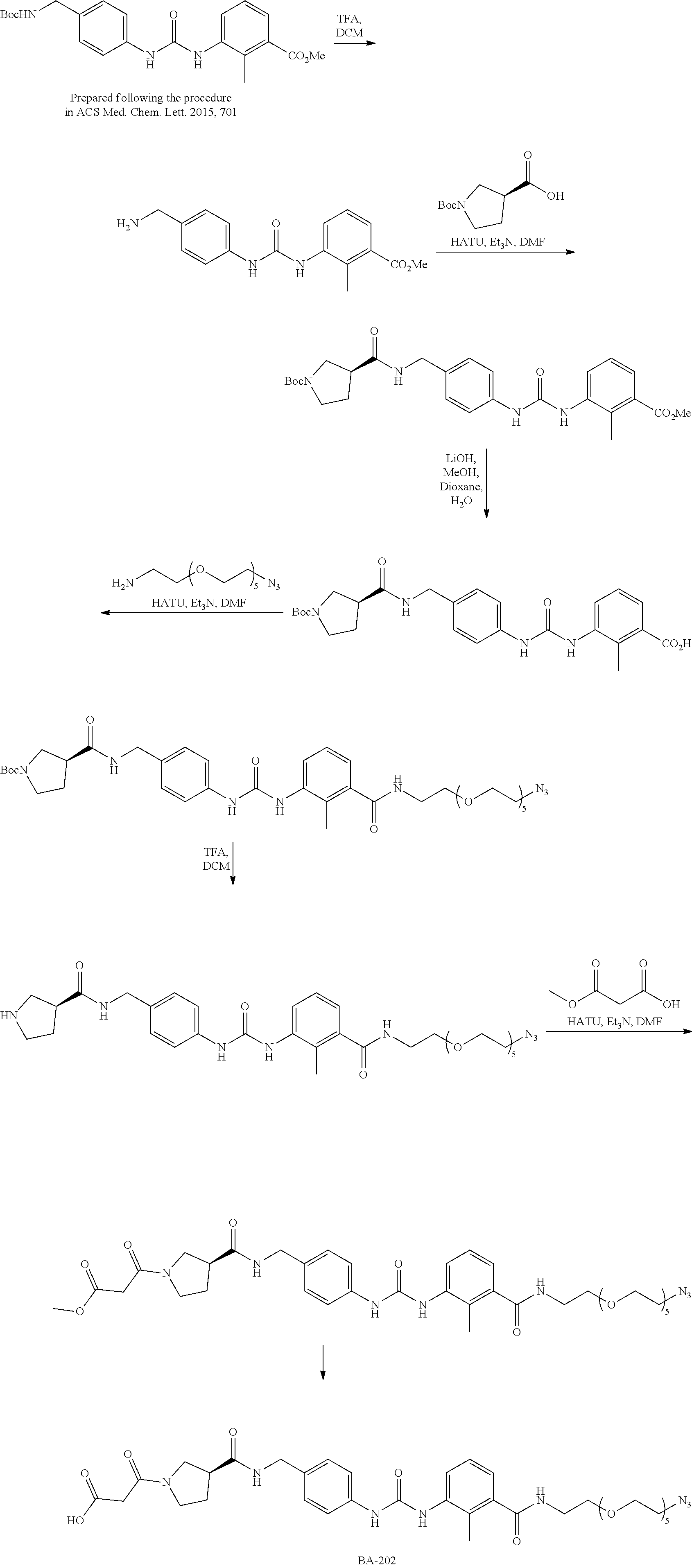

### Step 1: methyl 3-(3-(4-(aminomethyl)phenyl)ureido)-2-methylbenzoate

TFA (1 ml) was added dropwise at 0° C. to a solution of methyl 3-(3-(4-(((tert-butoxycarbonyl) amino) methyl)phenyl)ureido)-2-methylbenzoate (0.56 g, 1.355 mmol) in DCM (10 ml). The cooling bath was removed, and the mixture was stirred for 16 hours at room temperature, then concentrated to afford quantitatively the titled compound as a yellow oil. MS (ESI) m/z 314.4 [M+H]⁺.

### Step 2: tert-butyl (S)-3-((4-(3-(3-(methoxycarbonyl)-2-methylphenyl)ureido)benzyl) carbamoyl) pyrrolidine-1-carboxylate

To a solution of methyl 3-(3-(4-(aminomethyl)phenyl)ureido)-2-methylbenzoate (0.45 g, 1.09 mmol) in anhydrous DMF (5 ml), (S)-1-(tert-butoxycarbonyl)pyrrolidine-3-carboxylic acid (0.26 g, 1.2 mmol), HATU (0.54 g, 1.41 mmol) and Et₃N (0.31 mL, 2.18 mmol) were added. The reaction mixture was stirred at room temperature under inert atmosphere for 4 hours. Reaction mixture was diluted with water (40 ml) and extracted with DCM (3×20 mL). The combined organic extracts were washed with brine (25 ml), dried over anhydrous Na₂SO₄ and concentrated under reduced pressure. The crude material was purified by silica gel column chromatography using a gradient 50-100% EtOAc in hexane to afford the titled compound (0.20 g, 37%) as a yellow oil. MS (ESI) m/z 534.1 [M+Na]⁺.

### Step 3: 3-(3-(4-(((S)-1-(tert-butoxycarbonyl)pyrrolidine-3-carboxamido)methyl)cyclohexa-2,4-dien-1-yl)ureido)-2-methylbenzoic acid

To a solution of tert-butyl (S)-3-((4-(3-(3-(methoxycarbonyl)-2-methylphenyl)ureido)benzyl) carbamoyl) pyrrolidine-1-carboxylate (0.20 g, 0.392 mmol) in a methanol/water/dioxane (3 ml, 1:1:1) mixture, LiOH (56 mg, 2.35 mmol) was added and stirred at room temperature for 17 hours. The reaction mixture was concentrated under reduced pressure. The residue was treated with 10% citric acid solution and extracted with DCM (3×20 mL). The combined organic extracts were washed with brine (5 ml), dried over anhydrous Na₂SO₄ and concentrated under reduced pressure to obtain the title compound as a white solid (0.15 g, 77%). MS (ESI) m/z 521.5 [M+Na]⁺.

### Step 4: tert-butyl (S)-3-((4-(3-(3-((17-azido-3,6,9,12,15-pentaoxaheptadecyl)carbamoyl)-2-methylphenyl) ureido)benzyl)carbamoyl)pyrrolidine-1-carboxylate

To a solution of 3-(3-(4-(((S)-1-(tert-butoxycarbonyl)pyrrolidine-3-carboxamido)methyl) cyclohexa-2,4-dien-1-yl)ureido)-2-methylbenzoic acid (0.15 g, 0.306 mmol) in anhydrous DMF (1.5 ml), 17-azido-3,6,9,12,15-pentaoxaheptadecan-1-amine (0.10 g, 0.33 mmol), HATU (0.17 g, 0.45 mmol) and Et₃N (0.085 ml, 0.612 mmol) were added. The reaction mixture was stirred at room temperature under inert atmosphere for 3 hours. Reaction mixture was diluted with water (10 ml) and extracted with DCM (3×20 ml). The combined organic extracts were washed with brine (10 ml), dried over anhydrous Na₂SO₄ and concentrated under reduced pressure. The crude material was purified by silica gel column chromatography using a gradient 0-10% MeOH in DCM to obtain the title compound (0.18 g 76%) as a clear oil. MS (ESI) m/z 786.1 [M+H]⁺.

### Step 5: (S)-N-(4-(3-(3-((17-azido-3,6,9,12,15-pentaoxaheptadecyl)carbamoyl)-2-methylphenyl)ureido )benzyl)pyrrolidine-3-carboxamide

TFA (0.4 ml) was added dropwise at 0° C. to a solution of tert-butyl (S)-3-((4-(3-(3-((17-azido-3,6,9,12,15-pentaoxaheptadecyl)carbamoyl)-2-methylphenyl) ureido)benzyl)carbamoyl) pyrrolidine-1-carboxylate (0.18 g, 0.229 mmol) in DCM (2 ml). The cooling bath was removed, and the mixture was stirred for 3 hours at room temperature, then concentrated to afford the titled compound quantitatively as a yellow oil. MS (ESI) m/z 685.9 [M+H]⁺.

### Step 6: methyl (S)-3-(3-((4-(3-(3-((17-azido-3,6,9,12,15-pentaoxaheptadecyl)carbamoyl)-2-methylphenyl) ureido)benzyl)carbamoyl)pyrrolidin-1-yl)-3-oxopropanoate

To a solution of (S)-N-(4-(3-(3-((17-azido-3,6,9,12,15-pentaoxaheptadecyl)carbamoyl)-2-methylphenyl) ureido)benzyl)pyrrolidine-3-carboxamide (0.16 g, 0.229 mmol) in anhydrous DMF (1.5 ml), 3-methoxy-3-oxopropanoic acid (54 mg, 0.46 mmol), HATU (0.19 g, 0.50 mmol) and Et₃N (0.1 mL, 0.69 mmol) were added. The reaction mixture was stirred at room temperature under inert atmosphere for 4 hours. Reaction mixture was diluted with water (15 ml) and extracted with EtOAc (3×20 ml). The combined organic extracts were washed with brine (10 ml), dried over anhydrous Na₂SO₄ and concentrated under reduced pressure. The crude material was purified by silica gel column chromatography using a gradient 0-20% MeOH in DCM to afford the titled compound (0.10 g, 56%) as a yellow oil. MS (ESI) m/z 786.1 [M+H]⁺.

### Step 7: (S)-3-(3-((4-(3-(3-((17-azido-3,6,9,12,15-pentaoxaheptadecyl)carbamoyl)-2-methylphenyl)ureido) benzyl)carbamoyl)pyrrolidin-1-yl)-3-oxopropanoic acid

To a solution of methyl (S)-3-(3-((4-(3-(3-((17-azido-3,6,9,12,15-pentaoxaheptadecyl) carbamoyl)-2-methylphenyl) ureido)benzyl)carbamoyl)pyrrolidin-1-yl)-3-oxopropanoate (0.10 g, 0.127 mmol) in a methanol/water/dioxane (1.5 mL, 1:1:1) mixture, LiGH (10 mg, 0.382 mmol) was added and stirred at room temperature for 1 hour. The reaction mixture was concentrated under reduced pressure. The residue was treated with 10% citric acid solution and extracted with DCM (3×20 ml). The combined organic extracts were washed with brine (5 ml), dried over anhydrous Na₂SO₄ and concentrated. The crude material was purified by silica gel column chromatography using a gradient 0-20% MeOH in DCM to afford the titled compound (60 mg, 63%) as a white solid. MS (ESI) m/z 771.9 [M+H]⁺. ¹H NMR (500 MHz, DMSO-d₆) δ 6 12.59 (s, 1H), 9.31 (d, J=10.2 Hz, 1H), 8.49 (dt, J=16.1, 5.8 Hz, 1H), 8.25 (t, J=5.7 Hz, 1H), 8.20 (d, J=6.1 Hz, 1H), 7.84 (d, J=8.1 Hz, 1H), 7.44-7.39 (m, 2H), 7.17-7.13 (m, 3H), 6.95 (dd, J=7.6, 1.3 Hz, 1H), 4.22 (d, J=5.8 Hz, 2H), 4.10 (d, J=5.7 Hz, 1H), 3.67 (dd, J=10.2, 7.9 Hz, 1H), 3.62-3.54 (m, 4H), 3.54-3.46 (m, 12H), 3.46-3.33 (m, 8H), 3.30-3.21 (m, 1H), 3.17 (d, J=3.7 Hz, 1H), 3.10-3.00 (m, 1H), 2.97 (q, J=7.8 Hz, 1H), 2.22 (s, 3H), 2.14-1.97 (m, 2H), 1.97-1.87 (m, 1H).

BA-202 will be conjugated to an oligo sense strand according to general procedure type I, IIA/B, and/or III.

### Example 10: BA-210 Conjugates

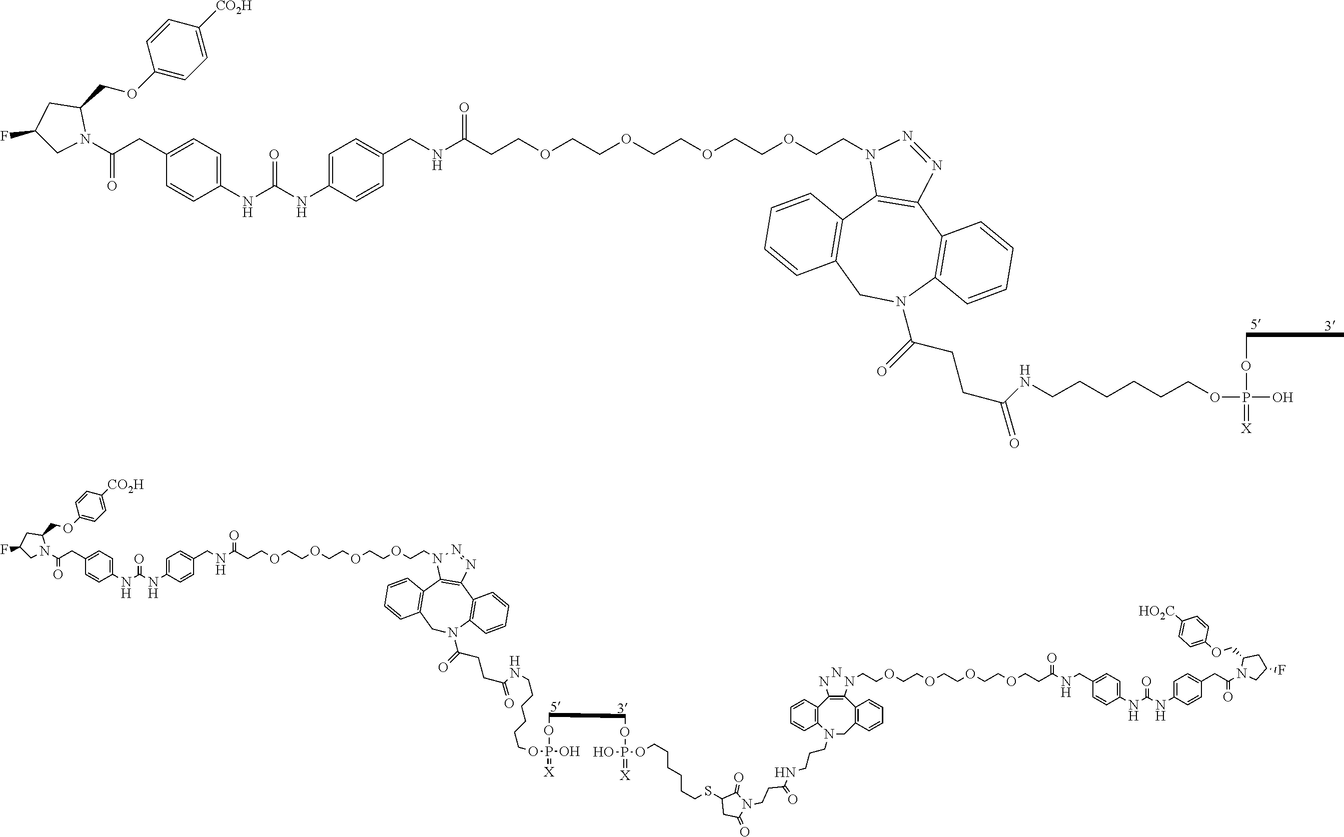

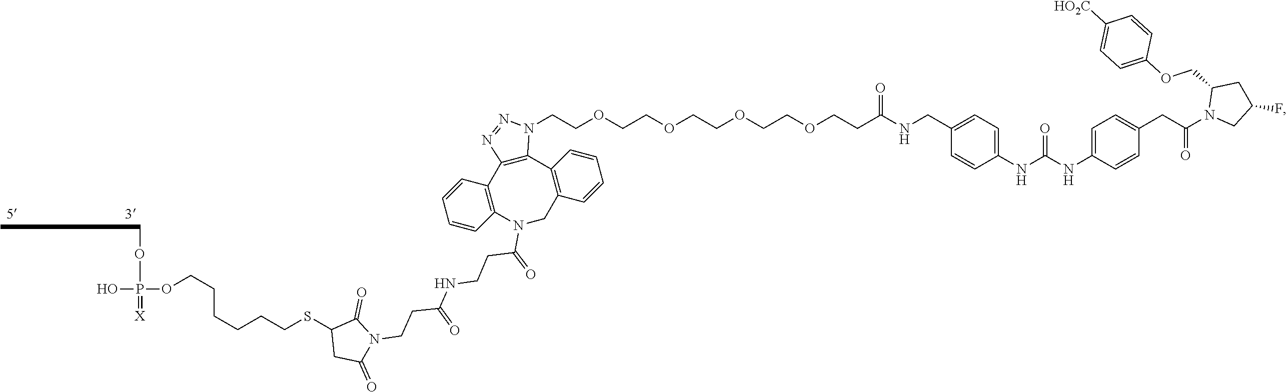

wherein X is O or S.

### BA-210: 4-(((2S,4S)-1-(2-(4-(3-(4-(17-azido-3-oxo-6,9,12,15-tetraoxa-2-azaheptadecyl)phenyl) ureido)phenyl)acetyl)-4-fluoropyrrolidin-2-yl)methoxy)benzoic acid

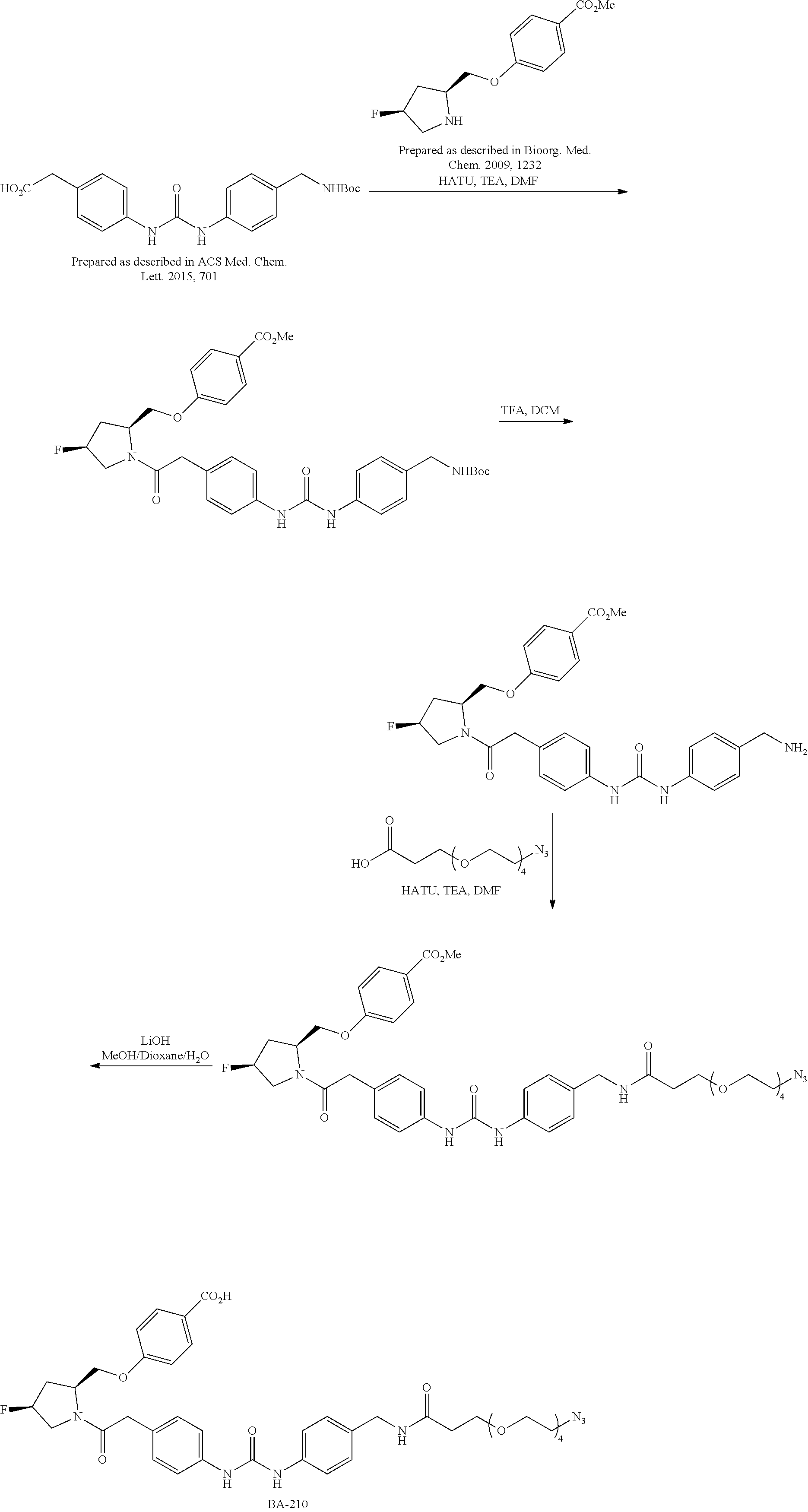

### Step 1: methyl 4-(((2S,4S)-1-(2-(4-(3-(4-(((tert-butoxycarbonyl)amino)methyl)phenyl)ureido) phenyl) acetyl)-4-fluoropyrrolidin-2-yl)methoxy)benzoate

To a solution of 2-(4-(3-(4-(((tert-butoxycarbonyl)amino)methyl)phenyl)ureido)phenyl)acetic acid (0.25 g, 0.626 mmol) in anhydrous DMF (3 ml), methyl 4-(((2S,4S)-4-fluoropyrrolidin-2-yl)methoxy)benzoate (0.23 g, 0.626 mmol), HATU (0.31 g, 0.814 mmol) and Et₃N (0.20 ml, 1.25 mmol) were added. The reaction mixture was stirred at room temperature under inert atmosphere for 3 hours. Reaction mixture was diluted with water (15 ml) and extracted with DCM (3×20 ml). The combined organic extracts were washed with brine (10 ml), dried over anhydrous Na₂SO₄ and concentrated under reduced pressure. The crude material was purified by silica gel column chromatography using a gradient 50-100% EtOAc in hexane to afford the titled compound (0.30 g, 76%) as a yellow oil. MS (ESI) m/z 657.8 [M+Na]⁺.

### Step 2: methyl 4-(((2S,4S)-1-(2-(4-(3-(4-(aminomethyl)phenyl)ureido)phenyl)acetyl)-4-fluoropyrrolidin-2-yl)methoxy)benzoate

TFA (0.3 ml) was added dropwise at 0° C. to a solution of methyl 4-(((2S,4S)-1-(2-(4-(3-(4-(((tert-butoxycarbonyl)amino)methyl)phenyl)ureido) phenyl) acetyl)-4-fluoropyrrolidin-2-yl)methoxy)benzoate (0.30 g, 0.473 mmol) in DCM (3 ml). The cooling bath was removed, and the mixture was stirred for 2 hours at room temperature, then concentrated to afford quantitatively the titled compound as a yellow oil. MS (ESI) m/z 1069.3 [2M]⁺.

### Step 3: methyl 4-(((2S,4S)-1-(2-(4-(3-(4-(17-azido-3-oxo-6,9,12,15-tetraoxa-2-azaheptadecyl) phenyl)ureido)phenyl)acetyl)-4-fluoropyrrolidin-2-yl)methoxy)benzoate

To a solution of methyl 4-(((2S,4S)-1-(2-(4-(3-(4-(aminomethyl)phenyl)ureido)phenyl)acetyl)-4-fluoropyrrolidin-2-yl)methoxy)benzoate (0.28 g, 0.43 mmol) in anhydrous DMF (3 ml), 1-azido-3,6,9,12-tetraoxapentadecan-15-oic acid (0.125 g, 0.43 mmol), HATU (0.21 g, 0.56 mmol) and Et₃N (0.18 ml, 1.29 mmol) were added. The reaction mixture was stirred at room temperature under inert atmosphere for 3 hours. Reaction mixture was diluted with water (20 ml) and extracted with DCM (3×20 ml). The combined organic extracts were washed with brine (10 ml), dried over anhydrous Na₂SO₄ and concentrated under reduced pressure. The crude material was purified by silica gel column chromatography using a gradient 50-100% EtOAc in hexane to afford the titled compound (0.20 g, 57%) as a yellow oil. MS (ESI) m/z 808.9 [M+H]⁺.

### Step 4: 4-(((2S,4S)-1-(2-(4-(3-(4-(17-azido-3-oxo-6,9,12,15-tetraoxa-2-azaheptadecyl)phenyl)ureido) phenyl)acetyl)-4-fluoropyrrolidin-2-yl)methoxy)benzoic acid

To a solution of methyl 4-(((2S,4S)-1-(2-(4-(3-(4-(17-azido-3-oxo-6,9,12,15-tetraoxa-2-azaheptadecyl) phenyl)ureido)phenyl)acetyl)-4-fluoropyrrolidin-2-yl)methoxy)benzoate (0.20 g, 0.248 mmol) in a methanol/water/dioxane (3 ml, 1:1:1) mixture, LiGH (20 mg, 0.744 mmol) was added and stirred at room temperature for 18 hours. The reaction mixture was concentrated under reduced pressure. The residue was treated with 10% citric acid solution and extracted with DCM (3×20 ml). The combined organic extracts were washed with brine (5 ml), dried over anhydrous Na₂SO₄ and concentrated to obtain the titled compound (0.19 g, 97%) as a white solid. MS (ESI) m/z 816.9 [M+Na]⁺. ¹H NMR (500 MHz, DMSO-d₆) δ 12.32 (s, 1H), 8.60 (s, 1H), 8.59 (s, 1H), 8.28 (t, J=5.9 Hz, 1H), 7.89-7.85 (m, 1H), 7.38 (d, J=8.5 Hz, 4H), 7.15 (d, J=8.4 Hz, 2H), 7.10-7.06 (m, 1H), 4.44-4.35 (m, 1H), 4.20 (d, J=5.9 Hz, 2H), 3.94-3.76 (m, 3H), 3.67-3.46 (m, 17H), 3.40-3.35 (m, 3H), 2.75 (d, J=15.4 Hz, 2H), 2.65 (d, J=15.4 Hz, 2H), 2.37 (t, J=6.4 Hz, 2H), 2.32-2.22 (m, 2H), 1.91 (s, 2H).

### BA-210 will be conjugated to an oligo sense strand according to general procedure type I, IIA/B, and/or III

### Example 11: BA-211 Conjugates

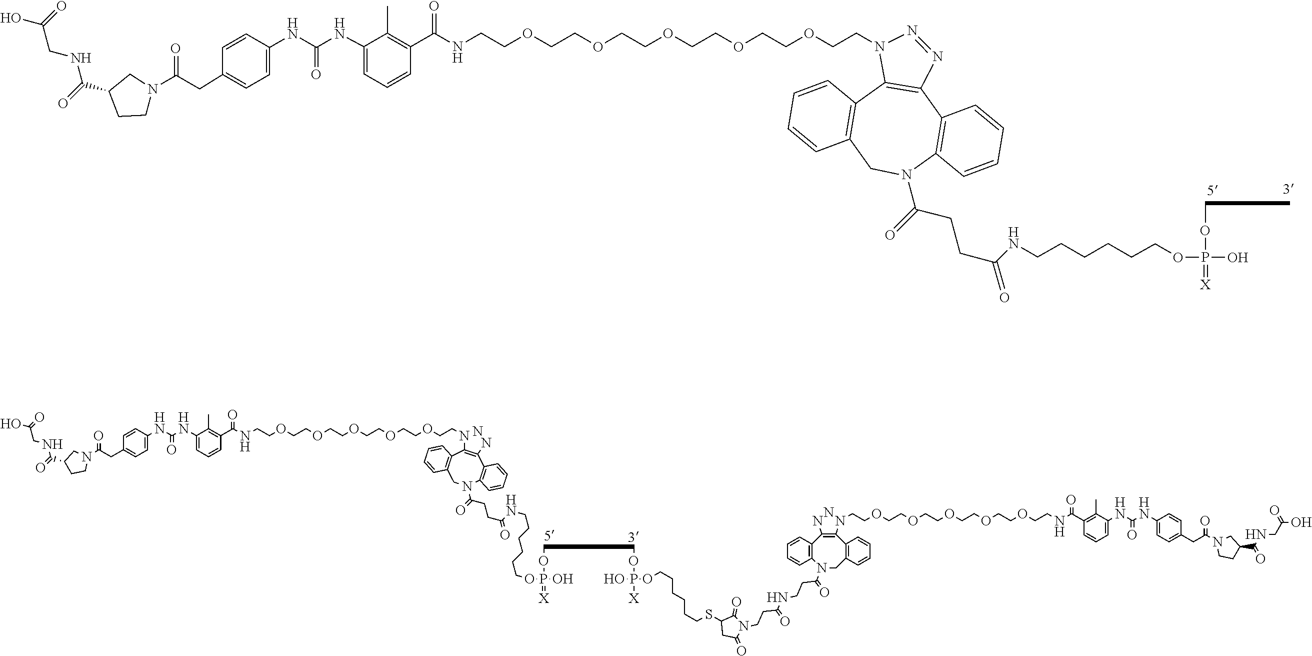

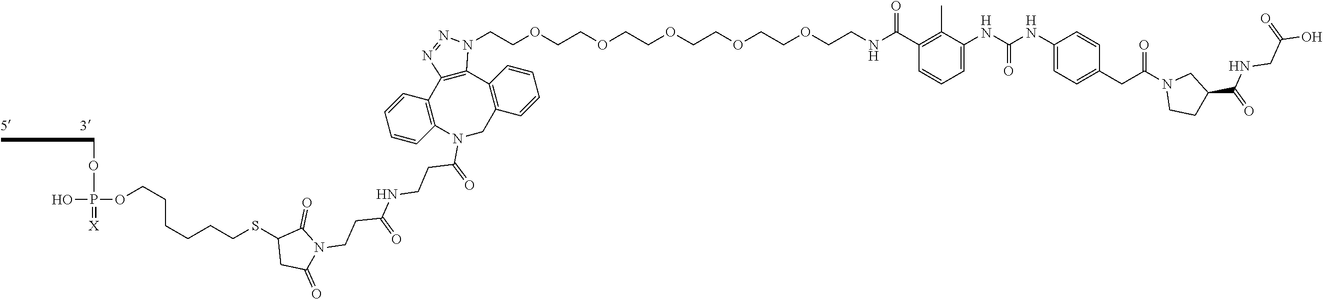

wherein X is O or S.

### BA-211: (S)-(1-(2-(4-(3-(3-((17-azido-3,6,9,12,15-pentaoxaheptadecyl)carbamoyl)-2-methylphenyl) ureido)phenyl)acetyl)pyrrolidine-3-carbonyl)glycine

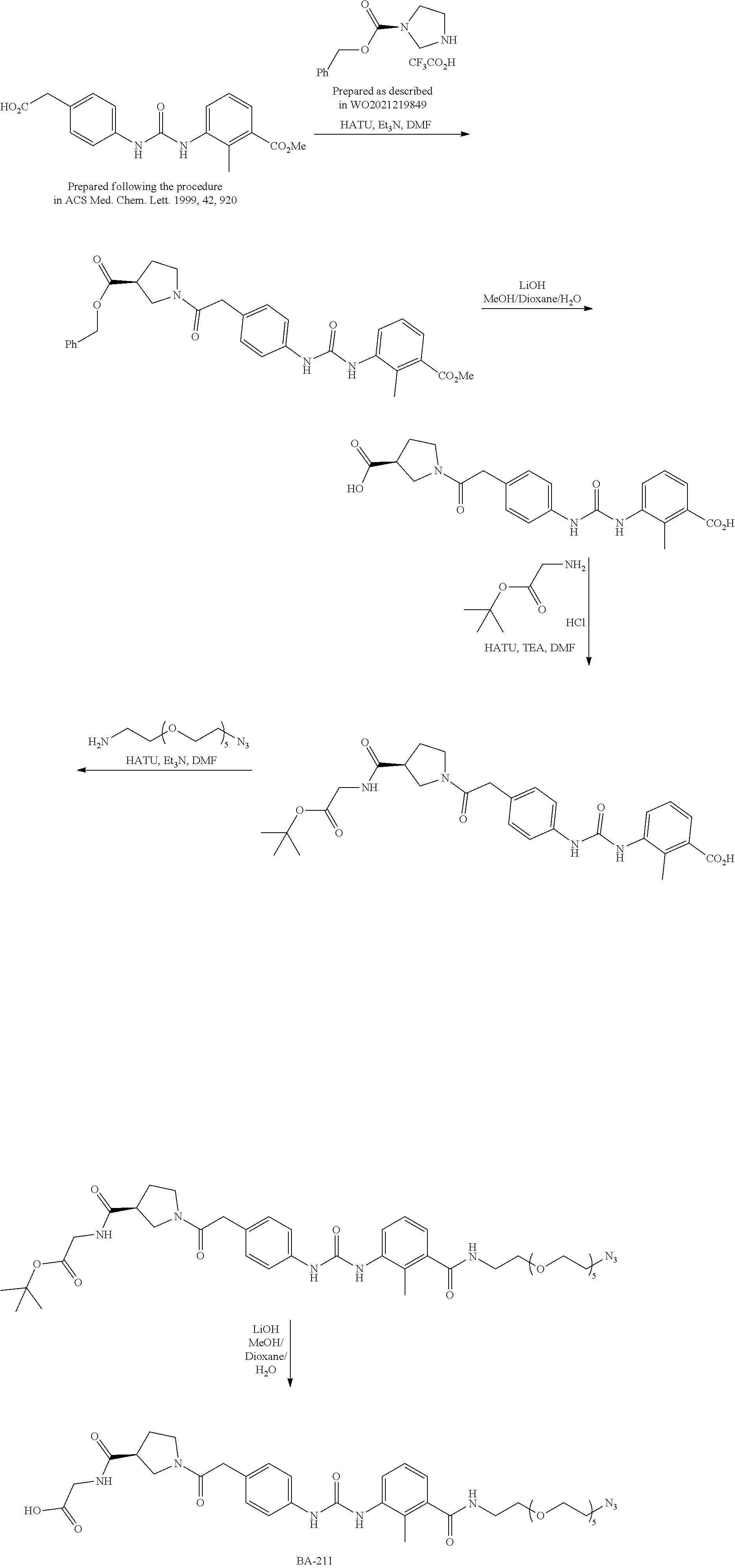

### Step 1: benzyl (S)-1-(2-(4-(3-(3-(methoxycarbonyl)-2-methylphenyl)ureido)phenyl)acetyl) pyrrolidine-3-carboxylate

To a solution of 2-(4-(3-(3-(methoxycarbonyl)-2-methylphenyl)ureido)phenyl)acetic acid (0.71 g, 2.074 mmol) in anhydrous DMF (10 ml), benzyl imidazolidine-1-carboxylate (0.66 g, 2.10 mmol), HATU (1 g, 2.7 mmol) and Et₃N (0.60 ml, 4.15 mmol) were added. The reaction mixture was stirred at room temperature under inert atmosphere for 3 hours. Reaction mixture was diluted with water (50 ml) and extracted with DCM (3×20 ml). The combined organic extracts were washed with brine (10 ml), dried over anhydrous Na₂SO₄ and concentrated under reduced pressure. The crude material was purified by silica gel column chromatography using a gradient 0-10% MeOH in DCM to afford the titled compound as a yellow solid (0.80 g, 70%). MS (ESI) m/z 530.7 [M+H]⁺.

### Step 2: (S)-1-(2-(4-(3-(3-carboxy-2-methylphenyl)ureido)phenyl)acetyl)pyrrolidine-3-carboxylic acid

To a solution of benzyl (S)-1-(2-(4-(3-(3-(methoxycarbonyl)-2-methylphenyl)ureido) phenyl) acetyl) pyrrolidine-3-carboxylate (0.70 g, 1.32 mmol) in a methanol/water/dioxane (4.5 ml, 1:1:1) mixture, LiOH (95 mg, 3.97 mmol) was added and stirred at room temperature for 2 hours. The reaction mixture was concentrated under reduced pressure. The residue was treated with 10% citric acid solution and purified by reverse-phase column chromatography (C18) using a gradient 0-40% ACN in water (+0.1% formic acid) to afford the titled compound as a white sticky solid (72 mg, 13%). MS (ESI) m/z 448.5 [M+Na]⁺.

### Step 3: (S)-3-(3-(4-(2-(3-((2-(tert-butoxy)-2-oxoethyl)carbamoyl)pyrrolidin-1-l)-²-oxoethyl)phenyl) ureido)-2-methylbenzoic acid

To a solution of (S)-1-(2-(4-(3-(3-carboxy-2-methylphenyl)ureido)phenyl)acetyl)pyrrolidine-3-carboxylic acid (72 mg, 0.169 mmol) in anhydrous DMF (1.5 ml), tert-butyl glycinate hydrochloride (28 mg, 0.169 mmol), HATU (83 mg, 0.22 mmol) and Et₃N (0.05 ml, 0.34 mmol) were added. The reaction mixture was stirred at room temperature under inert atmosphere for 3 hours. Reaction mixture was diluted with water (15 ml) and extracted with DCM (3×15 ml). The combined organic extracts were washed with brine (10 ml), dried over anhydrous Na₂SO₄ and concentrated under reduced pressure. The crude material was purified by silica gel column chromatography using a gradient 0-20% MeOH in DCM to obtain the titled compound as a yellow solid (70 mg, 79%). MS (ESI) m/z 561.7 [M+Na]⁺.

### Step 4: tert-butyl (S)-(1-(2-(4-(3-(3-((17-azido-3,6,9,12,15-pentaoxaheptadecyl)carbamoyl)-2-methyl phenyl)ureido)phenyl)acetyl)pyrrolidine-3-carbonyl)glycinate

To a solution of acid (70 mg, 0.13 mmol) in anhydrous DMF (1.5 ml), 17-azido-3,6,9,12,15-pentaoxa heptadecan-1-amine (40 mg, 0.13 mmol), HATU (64 mg, 0.169 mmol) and Et₃N (0.04 ml, 0.26 mmol) were added. The reaction mixture was stirred at room temperature under inert atmosphere for 3 hours. Reaction mixture was diluted with water (15 ml) and extracted with DCM (3×15 ml). The combined organic extracts were washed with brine (5 ml), dried over anhydrous Na₂SO₄ and concentrated under reduced pressure. The crude material was purified by silica gel column chromatography using a gradient 50-100% EtOAc in hexane to afford the titled compound as a yellow oil (0.11 g, 92%). MS (ESI) m/z 828.1 [M+H]⁺.

### Step 5: (S)-(1-(2-(4-(3-(3-((17-azido-3,6,9,12,15-pentaoxaheptadecyl)carbamoyl)-2-methylphenyl)ureido) phenyl)acetyl)pyrrolidine-3-carbonyl)glycine

To a solution of tert-butyl (S)-(1-(2-(4-(3-(3-((17-azido-3,6,9,12,15-pentaoxaheptadecyl) carbamoyl)-2-methylphenyl)ureido)phenyl)acetyl)pyrrolidine-3-carbonyl)glycinate (80 mg, 0.097 mmol) in a methanol/water/dioxane (1.5 mL, 1:1:1) mixture, LiGH (7 mg, 0.29 mmol) was added and stirred at room temperature for 5 hours. The reaction mixture was concentrated under reduced pressure. The residue was treated with 10% citric acid solution and purified by reverse-phase column chromatography (C18) using a gradient 0-40% ACN in water (+0.1% formic acid) to afford the titled compound as a white solid (42 mg, 56%). MS (ESI) m/z 771.9 [M+H]⁺. ¹H NMR (500 MHz, DMSO-d₆) δ 12.50 (s, 1H), 9.01 (s, 1H), 8.25 (t, J=5.7 Hz, 2H), 8.02 (s, 1H), 7.88-7.81 (m, 1H), 7.38 (dd, J=8.5, 3.9 Hz, 2H), 7.22-7.10 (m, 3H), 6.96 (dd, J=7.6, 1.3 Hz, 1H), 3.85 (d, J=6.0 Hz, 1H), 3.72 (dd, J=5.8, 2.2 Hz, 2H), 3.69-3.65 (m, 1H), 3.65-3.58 (m, 3H), 3.58-3.55 (m, 2H), 3.55-3.49 (m, 4H), 3.49-3.42 (m, 4H), 3.38 (ddd, J=8.1, 4.8, 2.6 Hz, 10H), 3.30-3.18 (m, 2H), 3.07 (p, J=7.7 Hz, 1H), 2.97 (h, J=7.2 Hz, 1H), 2.25 (s, 1H), 2.20 (s, 3H), 2.10-1.96 (m, 2H), 1.93-1.82 (m, 1H).

### BA-211 will be conjugated to an oligo sense strand according to general procedure type I, IIA/B, and/or III

### Example 12: BA-215 Conjugate

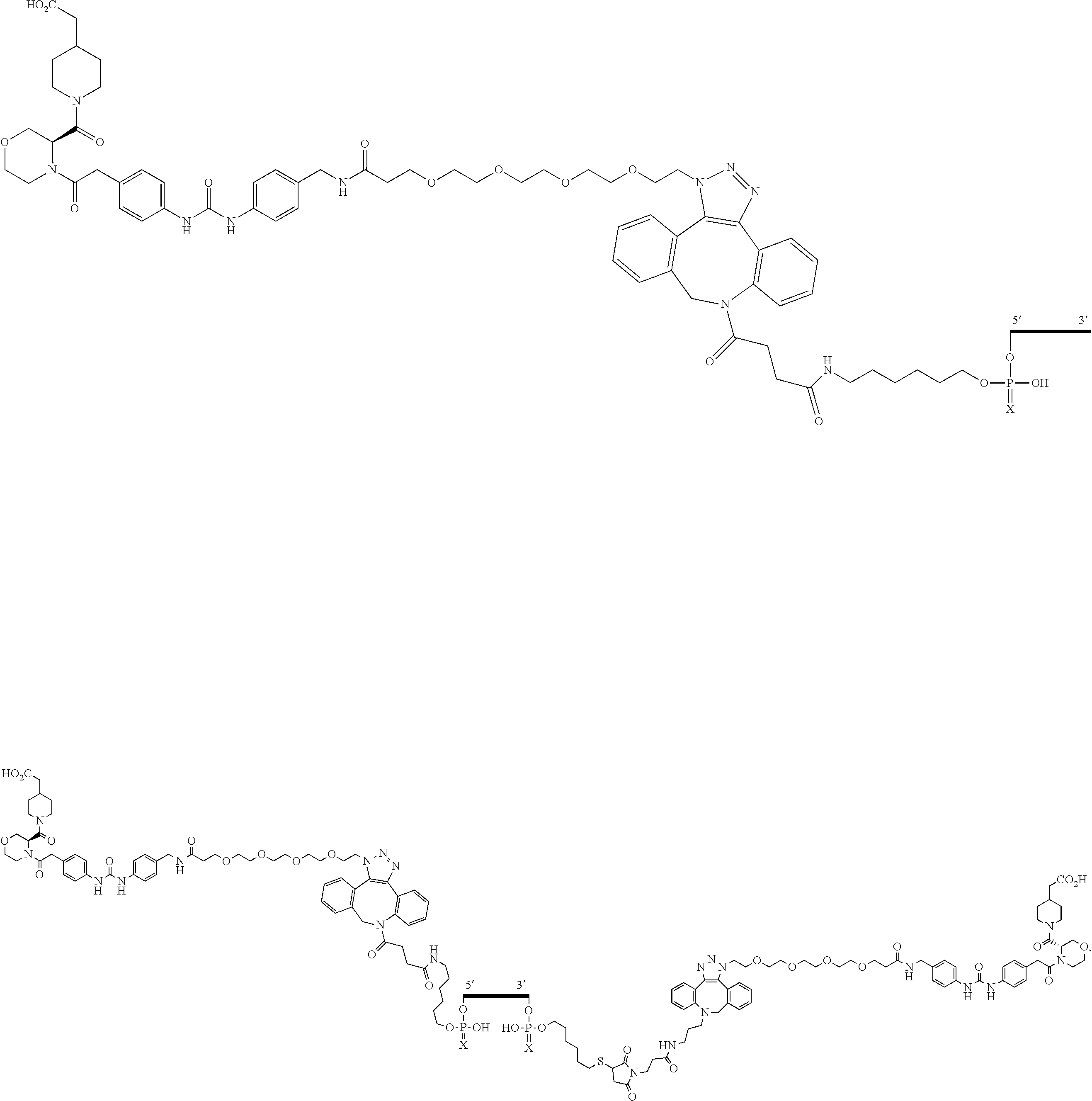

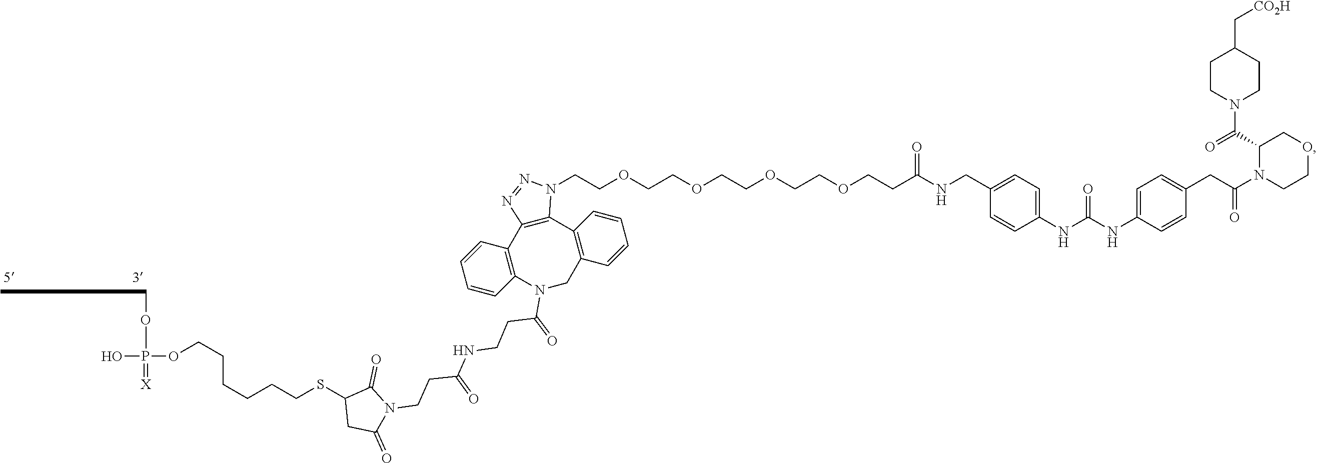

wherein X is O or S.

### BA-215: (S)-2-(1-(4-(2-(4-(3-(4-(17-azido-3-oxo-6,9,12,15-tetraoxa-2-azaheptadecyl)phenyl) ureido) phenyl)acetyl)morpholine-3-carbonyl)piperidin-4-yl)acetic acid

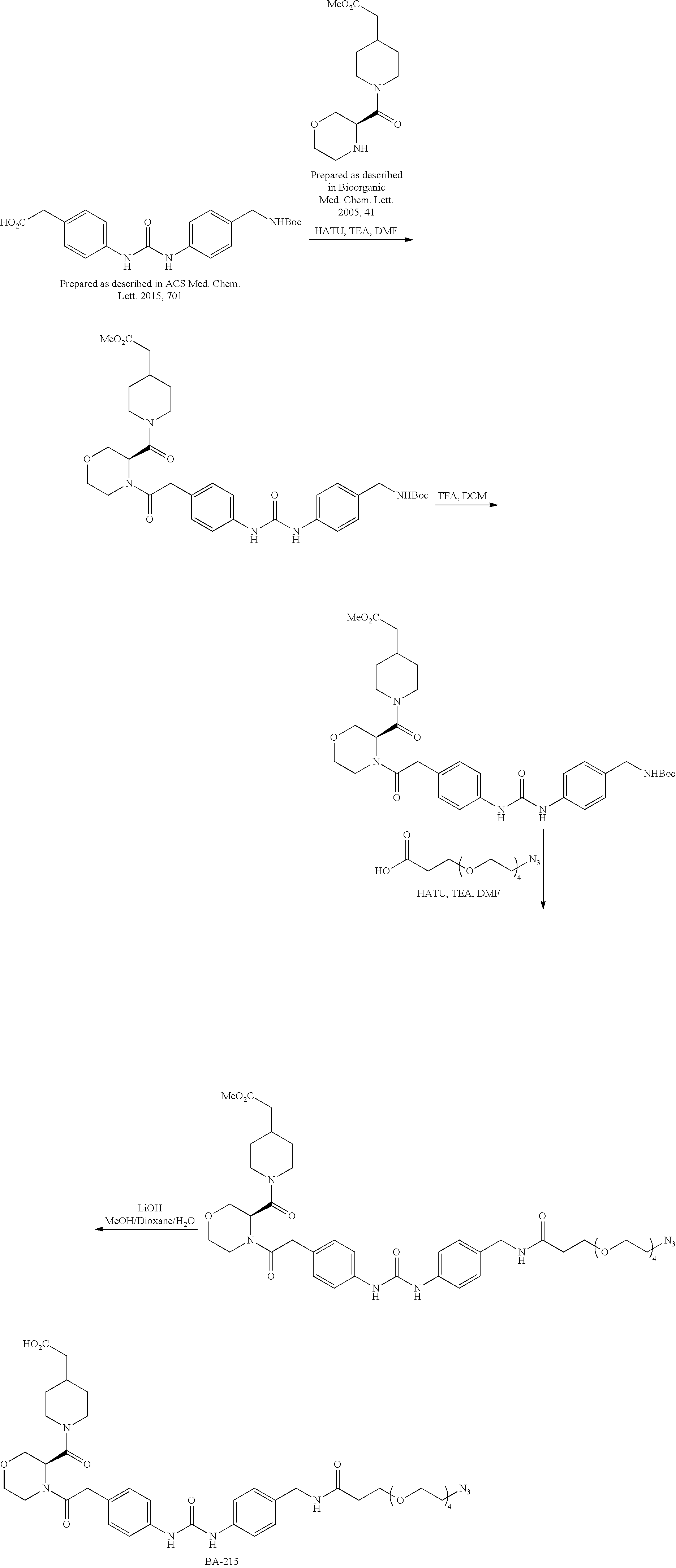

### Step 1: methyl (S)-2-(1-(4-(2-(4-(3-(4-(((tert-butoxycarbonyl)amino)methyl)phenyl)ureido) phenyl)acetyl) morpholine-3-carbonyl)piperidin-4-yl)acetate

To a solution of 2-(4-(3-(4-(((tert-butoxycarbonyl)amino)methyl)phenyl)ureido)phenyl)acetic acid (0.15 g, 0.36 mmol) in anhydrous DMF (3 ml), methyl (S)-2-(1-(morpholine-3-carbonyl)piperidin-4-yl)acetate (97 mg, 0.36 mmol), HATU (0.18 g, 0.47 mmol) and Et₃N (0.10 ml) were added. The reaction mixture was stirred at room temperature under inert atmosphere for 3 hours. Reaction mixture was diluted with water (20 ml) and extracted with EtOAc (3×20 ml). The combined organic extracts were washed with brine (25 ml), dried over anhydrous Na₂SO₄ and concentrated under reduced pressure. The crude material was purified by silica gel column chromatography using a gradient 0-20% MeOH in DCM to afford the titled compound (0.18 g, 57%) as a yellow oil. MS (ESI) m/z 652.6 [M+H]⁺.

### Step 2: methyl (S)-2-(1-(4-(2-(4-(3-(4-(aminomethyl)phenyl)ureido)phenyl)acetyl)morpholine-3-carbonyl)piperidin-4-yl)acetate

TFA (0.3 ml) was added dropwise at 0° C. to a solution of methyl (S)-2-(1-(4-(2-(4-(3-(4-(((tert-butoxycarbonyl)amino)methyl)phenyl)ureido)phenyl)acetyl) morpholine-3-carbonyl)piperidin-4-yl)acetate (0.18 g, 0.276 mmol) in DCM (2 ml). The cooling bath was removed, and the mixture was stirred for 5 hours at room temperature, then concentrated to afford quantitatively the titled compound as a yellow oil. MS (ESI) m/z 552.6 [M+H]⁺.

### Step 3: methyl (S)-2-(1-(4-(2-(4-(3-(4-(17-azido-3-oxo-6,9,12,15-tetraoxa-2-azaheptadecyl)phenyl) ureido)phenyl)acetyl)morpholine-3-carbonyl)piperidin-4-yl)acetate

To a solution of methyl (S)-2-(1-(4-(2-(4-(3-(4-(aminomethyl)phenyl)ureido)phenyl)acetyl) morpholine-3-carbonyl)piperidin-4-yl)acetate (0.18 g, 0.276 mmol) in anhydrous DMF (1.5 ml), 1-azido-3,6,9,12-tetraoxapentadecan-15-oic acid (80 mg, 0.276 mmol), HATU (0.14 g, 0.36 mmol) and Et₃N (0.20 ml) were added. The reaction mixture was stirred at room temperature under inert atmosphere for 3 hours. Reaction mixture was diluted with water (20 ml) and extracted with DCM (3×20 ml). The combined organic extracts were washed with brine (15 ml), dried over anhydrous Na₂SO₄ and concentrated under reduced pressure. The crude material was purified by silica gel column chromatography using a gradient 0-20% MeOH in DCM to obtain the title compound (0.11 g, 48%) as a yellow oil. MS (ESI) m/z 825.5 [M+H]⁺.

### Step 4: (S)-2-(1-(4-(2-(4-(3-(4-(17-azido-3-oxo-6,9,12,15-tetraoxa-2-azaheptadecyl)phenyl)ureido) phenyl)acetyl)morpholine-3-carbonyl)piperidin-4-yl)acetic acid

To a solution of methyl (S)-2-(1-(4-(2-(4-(3-(4-(17-azido-3-oxo-6,9,12,15-tetraoxa-2-azaheptadecyl) phenyl)ureido)phenyl)acetyl)morpholine-3-carbonyl)piperidin-4-yl)acetate (0.11 g, 0.248 mmol) in a mixture of methanol/water/dioxane (1.5 ml, 1:1:1), LiGH (10 mg, 0.40 mmol) was added and stirred at room temperature for 5 hours. The solvents were evaporated in vacuo. The residue was treated with 10% citric acid solution and purified by reverse-phase column chromatography (C18) using a gradient 0-40% ACN in water (+0.1% formic acid) to afford the titled compound as a white solid (56 mg, 51%). MS (ESI) m/z 811.9 [M+H]⁺. ¹H NMR (500 MHz, DMSO-d₆) δ 12.13 (s, 1H) 8.64 (s, 1H), 8.61 (s, 1H), 8.28 (t, J=5.9 Hz, 1H), 7.41-7.32 (m, 4H), 7.18-7.09 (m, 4H), 5.12 (s, 1H) 4.20 (d, J=5.8 Hz, 2H), 3.87-3.72 (m, 4H), 3.68 -3.52 (m, 13H), 3.52-3.46 (m, 10H), 3.38 (dd, J=5.6, 4.3 Hz, 3H), 2.57 (s, 1H), 2.37 (t, J=6.4 Hz, 2H), 2.21-2.08 (m, 2H), 2.15 (s, 2H), 1.68 (s, 2H).

### BA-215 will be conjugated to an oligo sense strand according to general procedure type I, IIA/B, and/or III

### Example 13: BA-219 Conjugate

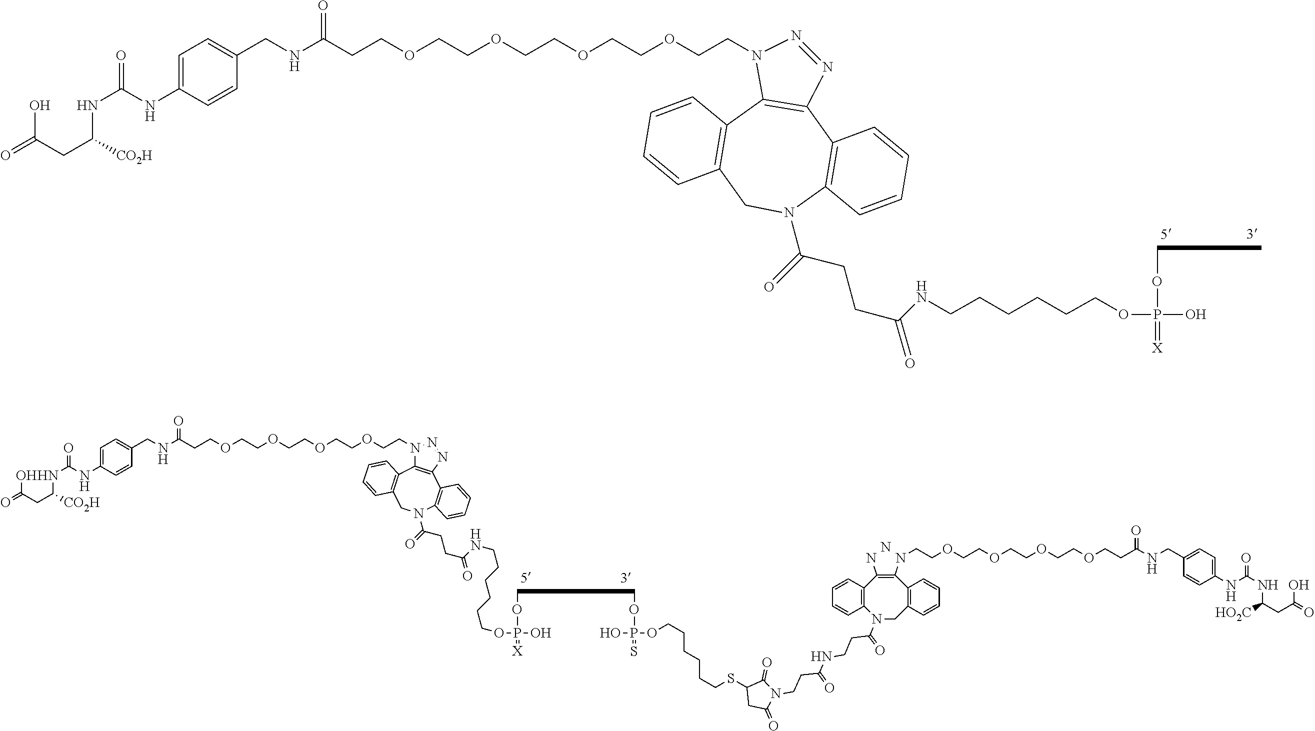

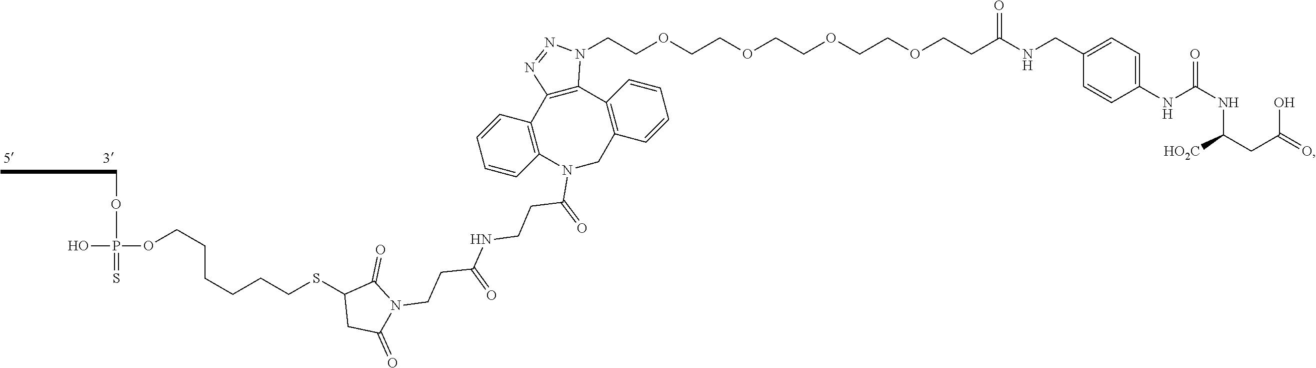

wherein X is O or S.

### BA-219: ((4-(17-azido-3-oxo-6,9,12,15-tetraoxa-2-azaheptadecyl)phenyl)carbamoyl)-L-aspartic acid

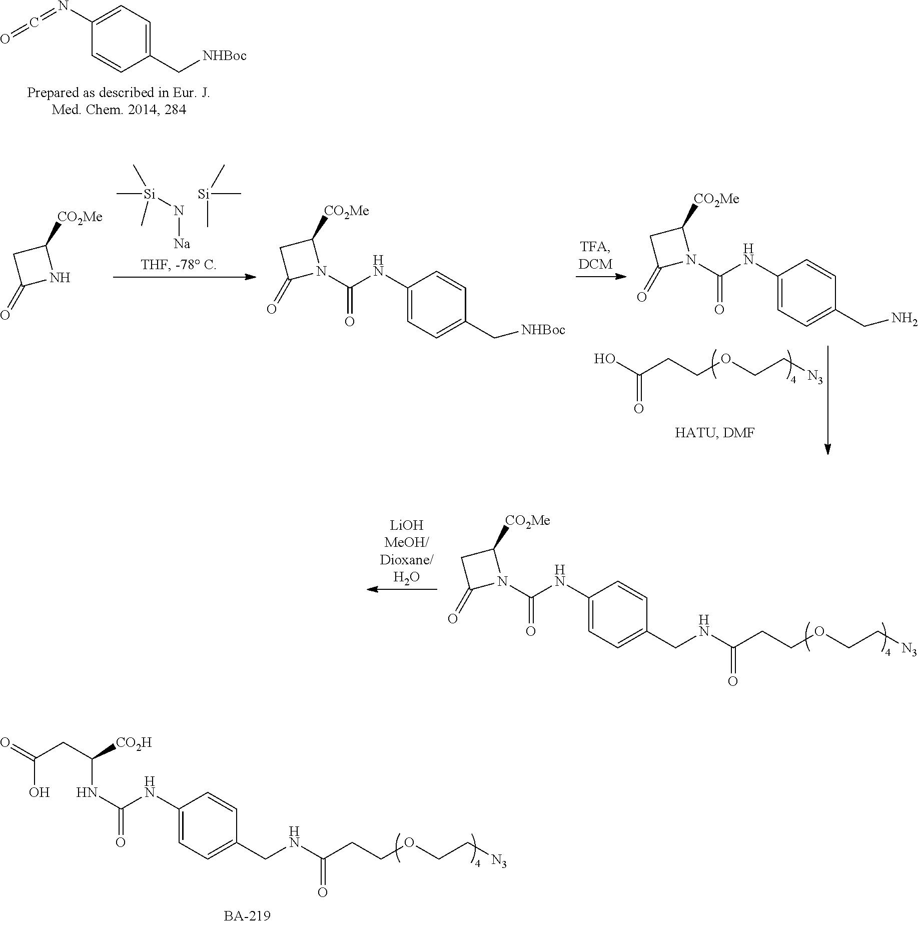

### Step 1: methyl (S)-1-((4-(((tert-butoxycarbonyl)amino)methyl)phenyl)carbamoyl)-4-oxoazetidine-2-carboxylate

A solution of sodium bis(trimethylsilyl)amide (2.98 ml, 2.98 mmol, 1.0 M in THF) was added dropwise to a solution of methyl (S)-4-oxoazetidine-2-carboxylate (0.35 g, 2.71 mmol) in anhydrous THF (30 ml) at −78° C. under a nitrogen atmosphere. The mixture was vigorously stirred for 25 min, then a solution of freshly prepared tert-butyl (4-isocyanatobenzyl)carbamate (0.87 g, 3.52 mmol ) in anhydrous THF (5 ml) was added dropwise. The reaction mixture was stirred at −78° C. for 2 hours and then quenched with a saturated solution of NH₄Cl (15 ml). Extracted with EtOAc (3×20 ml). The combined organic extracts were dried over sodium sulfate and concentrated. The crude residue was purified by silica gel column chromatography using a gradient 20-80% ethyl acetate in hexane to obtain the titled compound as a yellow oil (0.50 g 49 %). MS (ESI) m/z 400.4 [M+Na]⁺.

### Step 2. methyl (S)-1-((4-(aminomethyl)phenyl)carbamoyl)-4-oxoazetidine-2-carboxylate

TFA (1 ml) was added dropwise at 0° C. to a solution of methyl (S)-1-((4-(((tert-butoxycarbonyl)amino)methyl)phenyl)carbamoyl)-4-oxoazetidine-2-carboxylate (0.50 g, 1.32 mmol) in DCM (10 ml). The cooling bath was removed, and the mixture was stirred for 17 hours at room temperature, then concentrated to afford quantitatively the titled compound as a yellow oil. MS (ESI) m/z 278.4[M+H]⁺.

### Step 3: methyl (S)-1-((4-(17-azido-3-oxo-6,9,12,15-tetraoxa-2-azaheptadecyl)phenyl)carbamoyl) -4-oxoazetidine-2-carboxylate

To a solution of methyl (S)-1-((4-(aminomethyl)phenyl)carbamoyl)-4-oxoazetidine-2-carboxylate 2,2,2-trifluoroacetate (0.52 g, 1.33 mmol) in anhydrous DMF (6 ml), 1-azido-3,6,9,12-tetraoxapentadecan-15-oic acid (0.39 g, 1.33 mmol), HATU (0.66 g, 1.72 mmol) and Et₃N (0.37 ml, 2.66 mmol) were added. The reaction mixture was stirred at room temperature under inert atmosphere for 3 hours. Reaction mixture was diluted with water (40 ml) and extracted with DCM (3×25 ml). The combined organic extracts were washed with brine (10 ml), dried over anhydrous Na₂SO₄ and concentrated under reduced pressure. The crude material was purified by silica gel column chromatography using a gradient 50-100% EtOAc in hexane to afford the titled compound (0.46 g, 63%) as a yellow oil. MS (ESI) m/z 551.7 [M+H]⁺.

### Step 4: ((4-(17-azido-3-oxo-6,9,12,15-tetraoxa-2-azaheptadecyl)phenyl)carbamoyl)-L-aspartic acid

To a solution of methyl (S)-1-((4-(17-azido-3-oxo-6,9,12,15-tetraoxa-2-azaheptadecyl)phenyl) carbamoyl) -4-oxoazetidine-2-carboxylate (0.20 g, 0.392 mmol) in a methanol/water/dioxane (1.5 mL, 1:1:1) mixture, LiGH (28 mg, 1.17 mmol) was added and stirred at room temperature for 3 hours. The solvents were evaporated under vacuum. The residue was treated with 10% citric acid solution and purified by reverse-phase column chromatography (C18) using a gradient 0-40% ACN in water (+0.1% formic acid) to afford the titled compound as a white solid (190 mg, 97%). MS (ESI) m/z 555.6 [M+H]⁺. ¹H NMR (500 MHz, DMSO-d₆) δ 12.32 (bs, 2H), 8.60 (s, 1H), 7.93 (s, 1H), 7.34-7.28 (m, 2H), 7.14-7.09 (m, 2H), 6.39 (d, J=8.3 Hz, 1H), 4.51 (t, J=6.4 Hz, 1H), 4.20 (d, J=5.9 Hz, 2H), 3.71-3.60 (m, 4H), 3.60-3.48 (m, 10H), 3.39 (t, J=5.1 Hz, 2H), 3.20 (s, 2H), 2.79-2.65 (m, 2H), 2.38 (t, J=6.5 Hz, 2H).

### BA-219 will be conjugated to an oligo sense strand according to general procedure type I, IIA/B, and/or III

### Example 14: Effect of RD2841 targeting rat CTNNB1 (Catenin Beta 1)

The compound RD2841 has the following structure attached to an siRNA targeting CTNNB1:

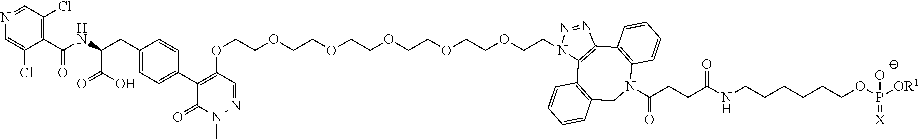

wherein X is O or S, and R¹ is the siRNA targeting CTNNB1 (i.e., X is O and R¹ is the covalently bound structure of compound RD2540 described below).

RD2841 was evaluated in an in vivo rat PD study. Six animals received a single 0.9 mg (3 mg/kg) dose via intrathecal injection on day 1. Animals were observed every day for behavioral changes. Frontal Cortex was collected from half the animals on day 15 and the other half on day 29. Tissue was immediately placed in homogenizing tube, snap frozen, then kept at −80° C. for gene expression analysis. The remainder of the cortex was collected for PK analysis.

RNA Isolation was performed according to the RNeasy Micro Kit (Qiagen Cat #74004) instructions. Following RNA isolation, a 96-well plate was placed on ice while the qRT-PCR reaction was prepared. 2 μl of RNA was added to the reaction mixture containing 5 pl TaqMan Fast Virus 1-Step Master Mix (Thermo Fisher #44444432), 1 pl CTNNB1 TaqMan Gene Expression Assay (Thermo Fisher: Rn00584431_g1, FAM), 1 pl ACTB (VIC) TaqMan Gene Expression Assay (Thermo Fisher:Rn00667869_m1, VIC), and 11 pl RT-PCR grade nuclease-free water in a MicroAmp Optical 96-well plate (0.2 mL). qPCR was performed using a QuantStudio3 qPCR machine with the following cycles: 50° C. for 1 minute, 95° C. for 20 seconds, 40 cycles at 95° C. for 15 seconds, and 60° C. for 1 minute. Results are presented in the table below as percent inhibition of CTNNB1, relative to vehicle control.

TABLE 1
Average CTNNB1 Inhibition

| Compound | Day 15 | Day 29 |
|---|---|---|
| RD2841 | 64% | 39% |

### Example 15: Effect of RD2841 targeting rat CTNNB1 in various brain regions

The compound RD2841 as described above was evaluated in an in vivo rat PD study. Ten animals received a single 0.9 mg (3 mg/kg) dose via intrathecal injection on day 1. Animals were observed every day for behavioral changes. Brain regions were collected from half the animals on day 15 and the other half on day 29. Tissue was immediately placed in homogenizing tube, snap frozen, then kept at −80° C. for gene expression analysis. The remainder of the cortex was collected for PK analysis.

RNA Isolation and qPCR was performed as described in Example 14 above. Results for Day 15 are presented in the table below as percent inhibition of CTNNB1, relative to vehicle control.

TABLE 2
Average CTNNB1 Inhibition by Compound RD2841

| Site | Day 15 | Day 29 |
|---|---|---|
| Striatum | 50% | 39% |
| Cerebellum | 58% | 53% |
| Brain Stem | 63% | 63% |
| Hippocampus | 71% | 64% |
| Frontal Cortex | 54% | 40% |
| Spinal Cord | 67% | 67% |

### Example 16: Effect of RD2540 targeting rat CTNNB1

The compound RD2540 has the structure described in the tables below. RD2540 is the siRNA used in Examples 14 and 15 above and is not conjugated to a targeting ligand. The activity of RD2540 was tested as a comparison to the targeting ligand-conjugated compound tested above.

TABLE 3
Chemical Nomenclature

| Abbreviation | Structure |
|---|---|
| v | 5′-vinylphosphonate modification |
| ‘m’ | 2′-O-methyl sugar modification (e.g., mA, mG, mC, mU) |
| ‘f’ | 2′-F sugar modification (e.g., fA, fG, fC, fU) |
| ‘*’ | Phosphorothioate internucleoside linkage |
| ‘dQ’ | Inverted abasic deoxyribose |

TABLE 4
Unconjugated Parent Compound

| | | Ref ID | SEQ ID |
|---|---|---|---|
| Compound | Modified Strands (5′-3′) | NO: | NO: |
| RD2540 | (vU)*(fU)*(mU)(mC)(mG)(fA)(mA)(fU)(fC)(mA) | IA0883 | 1 |
| | (mA)(mU)(mC)(fC)(mA)(fA)(mC)(mA)(mG)(mU) | | |
| | (mA)*(mG)*(mC) | | |
| | (mU)*(mA)*(mC)(mU)(mG)(mU)(fU)(mG)(fG)(fA) | IS1096 | 2 |
| | (fU)(mU)(mG)(mA)(mU)(mU)(mC)(mG)(mA) | | |
| | (mA)*(mA)*(dQ) | | |

RD2540 was evaluated in an in vivo rat PD study carried out as described in Example 14. Results are presented in the table below as percent inhibition of CTNNB1, relative to vehicle control.

TABLE 5
Average CTNNB1 Inhibition

| Compound | Day 15 | Day 29 |
|---|---|---|
| RD2540 | 31% | 13% |

### Example 17 (Target A rat #3, BA-128): Effect of Compound 1 targeting rat Target A in various brain regions

Compound 1 has an siRNA targeting Target A attached to an Integrin ligand as follows:

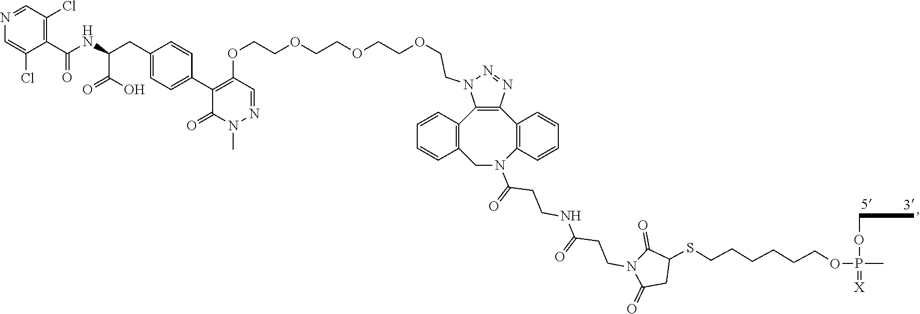

wherein X is S and R is the siRNA targeting Target A (i.e., R is the covalently bound structure of the siRNA).

Compound 1 was evaluated in an in vivo rat PD study. The animals received a single vehicle or 0.9 mg (3 mg/kg) dose via intrathecal injection on day 1 (n=3/group). Animals were observed every day for behavioral changes. Brain regions were collected on day 15, day 29, or day 43, and tissue was immediately placed in homogenizing tube, snap frozen, then kept at −80° C. for gene expression analysis.

RNA Isolation and qPCR was performed as described in Example 14 above, with the exception that instead of rat CTNNB1 TaqMan Gene Expression Assay, the rat Target A TaqMan Gene Expression Assay (Thermo Fisher) was used. Results are presented in Table 6 below as percent inhibition of Target A, relative to vehicle control.

TABLE 6
Average Target A Inhibition by Compound 1

| | Day | Day | Day |
|---|---|---|---|
| Brain Region | 15 | 29 | 43 |
| Striatum | 49% | 47% | 30% |
| Hippocampus | 68% | 80% | 80% |
| Frontal Cortex | 85% | 77% | 74% |
| Temporal Cortex | 84% | 66% | 75% |
| Cerebellum | 72% | 77% | 74% |
| Brainstem | 81% | 88% | 91% |
| Cervical Spinal Cord | 85% | 81% | 85% |
| Thoracic Spinal Cord | 84% | 83% | 84% |
| Lumbar Spinal Cord | 74% | 85% | 84% |

### Example 18 (Target A rat #7, BA-148): Effect of Compound 2 targeting rat Target A in various brain regions

Compound 2 has an siRNA targeting Target A attached to an Integrin ligand as follows:

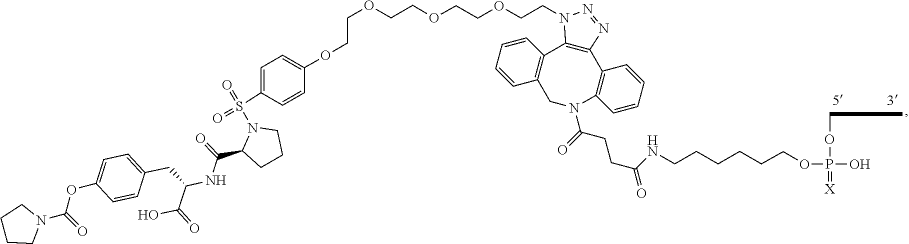

wherein X is S and R is the siRNA targeting Target A (i.e., R is the covalently bound structure of the siRNA).

Compound 2 was evaluated in an in vivo rat PD study carried out as described in Example 17. Results are presented in Table below as percent inhibition of Target A, relative to vehicle control.

TABLE 7
Average Target A Inhibition by Compound 2

| Brain Region | Day 15 |
|---|---|
| Striatum | 68% |
| Hippocampus | 87% |
| Frontal Cortex | 72% |

### Example 19 (Target A rat #7, BA-149): Effect of Compound 3 targeting rat Target A in various brain regions

Compound 3 has an siRNA targeting Target A attached to an Integrin ligand as follows:

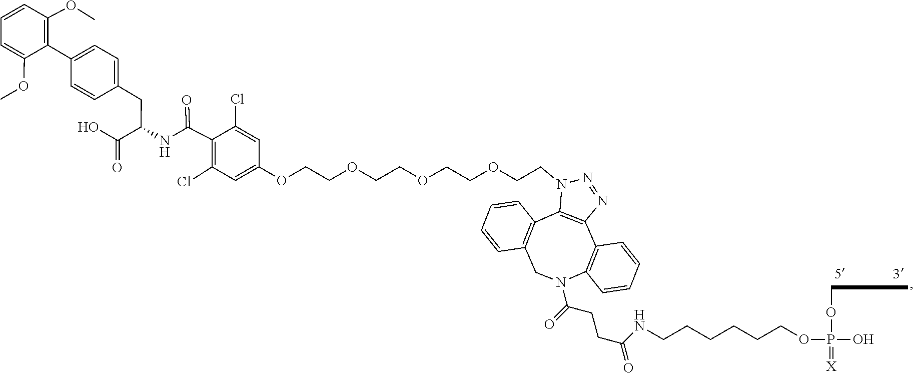

wherein X is S and R is the siRNA targeting Target A (i.e., R is the covalently bound structure of the siRNA).

Compound 3 was evaluated in an in vivo rat PD study carried out as described in Example 17. Results are presented in Table below as percent inhibition of Target A, relative to vehicle control.

TABLE 8
Average Target A Inhibition by Compound 3

| Brain Region | Day 15 |
|---|---|
| Striatum | 63% |
| Hippocampus | 88% |
| Frontal Cortex | 75% |

### Example 20 (Target A rat #7, BA-154): Effect of Compound 4 targeting rat Target A in various brain regions

Compound 4 has an siRNA targeting Target A attached to an Integrin ligand as follows:

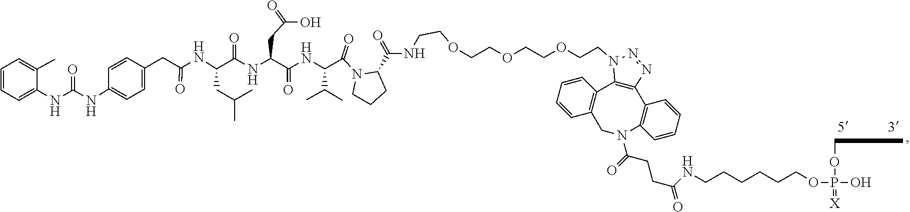

wherein X is S and R is the siRNA targeting Target A (i.e., R is the covalently bound structure of the siRNA).

Compound 4 was evaluated in an in vivo rat PD study carried out as described in Example 17. Results are presented in Table below as percent inhibition of Target A, relative to vehicle control.

TABLE 9
Average Target A Inhibition by Compound 4

| Brain Region | Day 15 |
|---|---|
| Striatum | 54% |
| Hippocampus | 84% |
| Frontal Cortex | 57% |

### Example 21 (Target B mouse #24, BA-171): Effect of Compound 5 targeting human Target B in various brain regions

Compound 5 has an siRNA targeting human Target B attached to an Integrin ligand as follows:

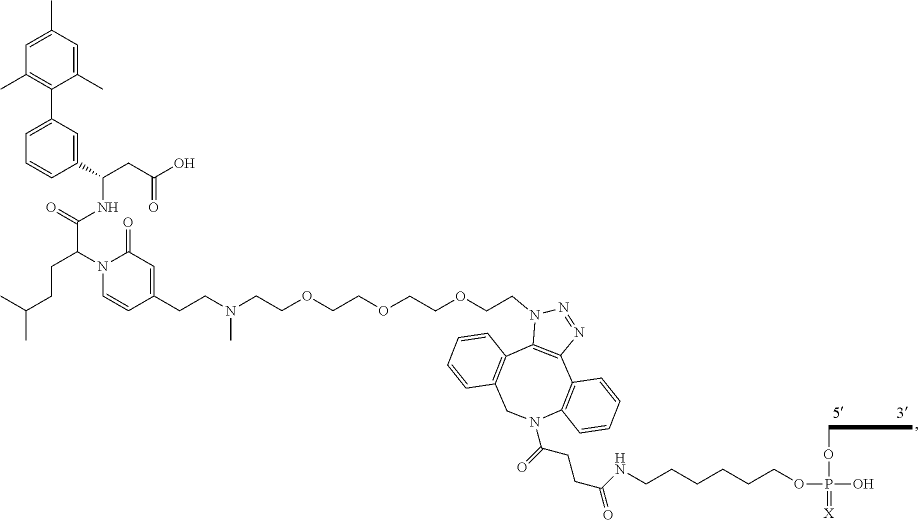

wherein X is S and R is the siRNA targeting Target B (i.e., R is the covalently bound structure of the siRNA).

Compound 5 was evaluated in an in vivo human Target B transgenic mice PD study. The animals received a single vehicle or 0.2 mg (10 mg/kg) dose via intracerebroventricular injection on day 1 (n=3/group). Animals were observed every day for behavioral changes. Brain regions were collected on day 15, and tissue was immediately placed in homogenizing tube, snap frozen, then kept at −80° C. for gene expression analysis.

RNA Isolation and qPCR was performed as described in Example 14 above, with the exceptions that the instead of rat CTNNB1 TaqMan Gene Expression Assay, the human Target B TaqMan Gene Expression Assay (Thermo Fisher) was used; instead of rat ACTB (VIC) TaqMan Gene Expression Assay, the mouse GAPDH TaqMan Gene Expression Assay (Thermo Fisher: Mm99999915_gI, VIC) was used. Results are presented in table below as percent inhibition of Target B, relative to vehicle control.

TABLE 10
Average Target B Inhibition by Compound 5

| Brain Region | Day 15 |
|---|---|
| Cortex | 69% |
| Hippocampus | 75% |
| Cerebellum | 52% |
| Striatum | 74% |
| Cervical Spinal Cord | 71% |

### Example 22 (Target B mouse #33, BA-128/BA-128): Effect of Compound 6 targeting human Target B in various brain regions

Compound 6 has an siRNA targeting human Target B attached to an Integrin ligand as follows:

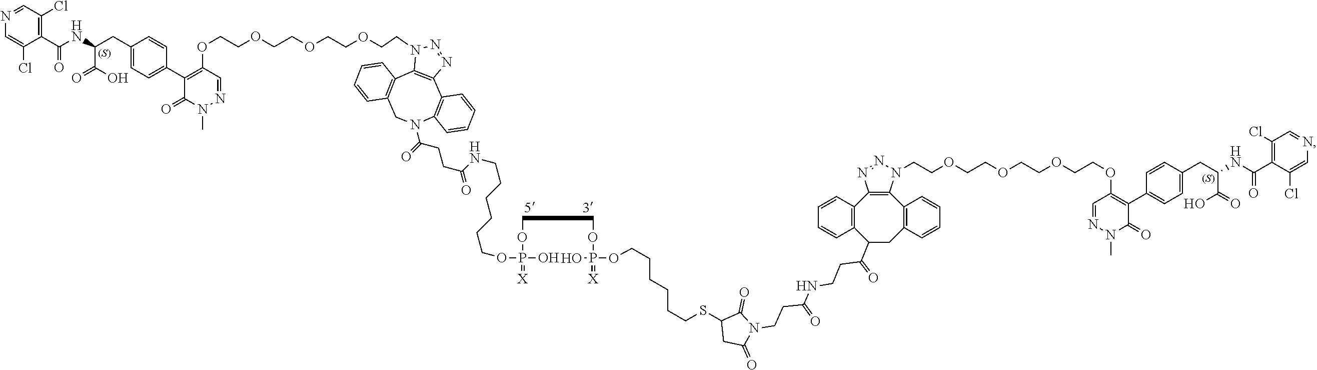

wherein X is S and R is the siRNA targeting Target B (i.e., R is the covalently bound structure of the siRNA).

Compound 6 was evaluated in an in vivo human Target B transgenic mice PD study carried out as described in Example 21. Results are presented in table below as percent inhibition of Target B, relative to vehicle control.

TABLE 11
Average Target B Inhibition by Compound 6

| Brain Region | Day 15 |
|---|---|
| Cortex | 56% |
| Hippocampus | 54% |
| Cerebellum | 36% |
| Striatum | 61% |
| Cervical Spinal Cord | 64% |

### Example 23 (Target B mouse #24, BA-172): Effect of Compound 7 targeting human Target B in various brain regions

Compound 7 has an siRNA targeting human Target B attached to the following Integrin ligand:

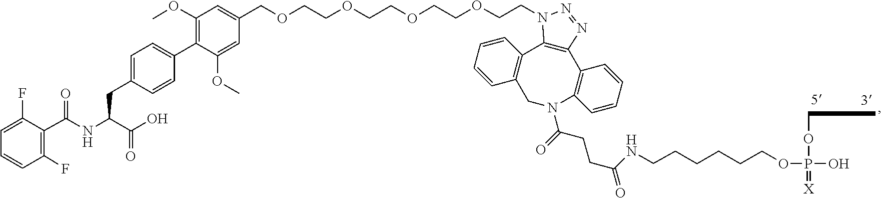

wherein X is S and R is the siRNA targeting Target B (i.e., R is the covalently bound structure of the siRNA).

Compound 7 was evaluated in an in vivo human Target B transgenic mice PD study carried out as described in Example 21. Results are presented in table below as percent inhibition of Target B, relative to vehicle control.

TABLE 12
Average Target B Inhibition by Compound 7

| Brain Region | Day 15 |
|---|---|
| Cortex | 52% |
| Hippocampus | 63% |
| Cerebellum | 47% |
| Striatum | 69% |
| Cervical Spinal Cord | 69% |

### Example 24 (Target B mouse #39, BA-128): Effect of Compound 8 targeting human Target B in various brain regions

Compound 8 has an siRNA targeting human Target B attached to an Integrin ligand as follows:

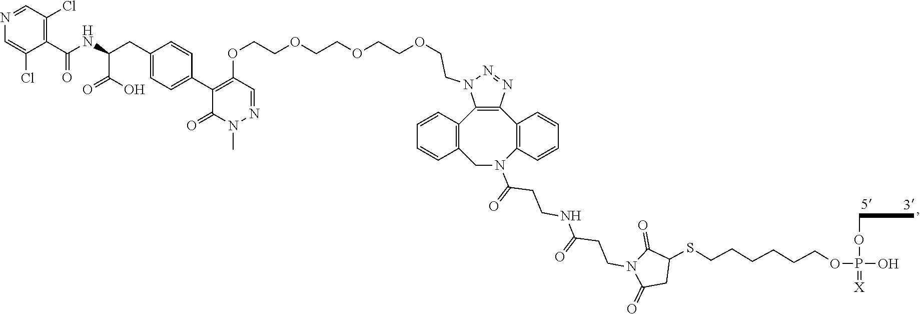

wherein X is S and R is the siRNA targeting Target B (i.e., R is the covalently bound structure of the unconjugated siRNA).

Compound 8 was evaluated in an in vivo human Target B transgenic mice PD study carried out as described in Example 21. Results are presented in table below as percent inhibition of Target B, relative to vehicle control.

TABLE 13
Average Target B Inhibition by Compound 8

| Brain Region | Day 15 |
|---|---|
| Cortex | 37% |
| Hippocampus | 39% |
| Cerebellum | 53% |
| Striatum | 51% |
| Cervical Spinal Cord | 57% |

### Example 25 (Target B mouse #57, BA-171/BA-171): Effect of Compound 9 targeting human Target B in various brain regions

Compound 9 has an siRNA targeting human Target B attached to an Integrin ligand as follows:

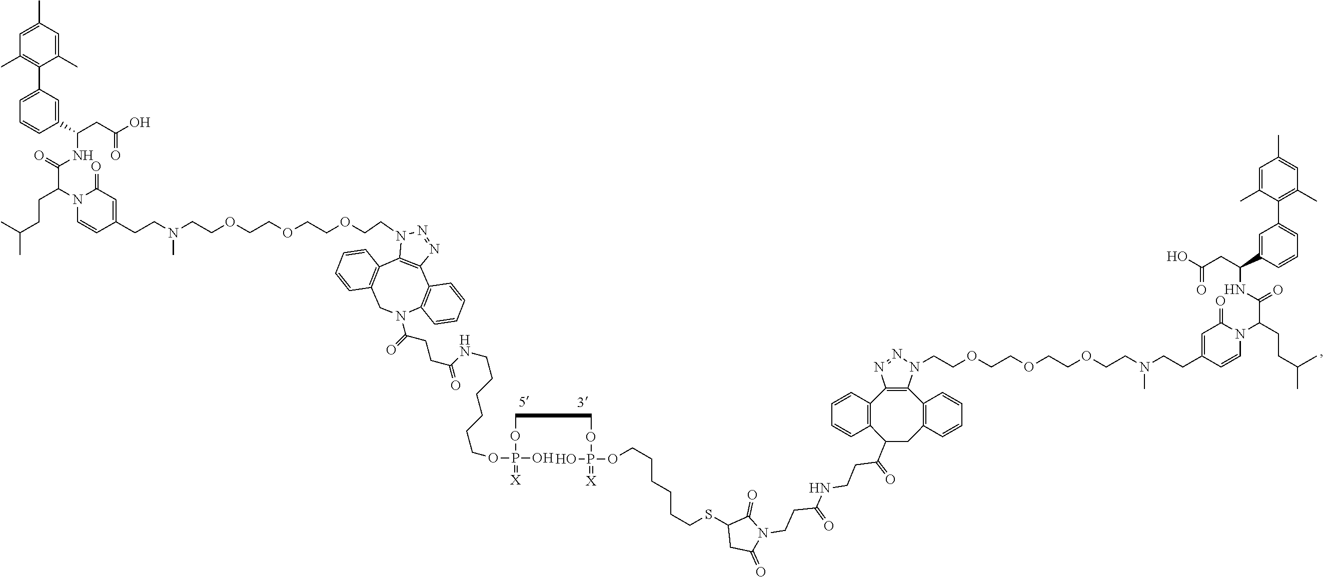

wherein X is S and R is the siRNA targeting Target B (i.e., R is the covalently bound structure of the siRNA).

Compound 9 was evaluated in an in vivo human Target B transgenic mice PD study carried out as described in Example 21. Results are presented in table below as percent inhibition of Target B, relative to vehicle control.

TABLE 14
Average Target B Inhibition by Compound 9

| Brain Region | Day 15 |
|---|---|
| Cortex | 72% |
| Hippocampus | 72% |
| Cerebellum | 52% |
| Striatum | 75% |
| Cervical Spinal Cord | 76% |

### Example 26 (Target B mouse #23, BA-161): Effect of Compound 10 targeting human Target B in various brain regions

Compound 10 has an siRNA targeting human Target B attached to an Integrin ligand as follows:

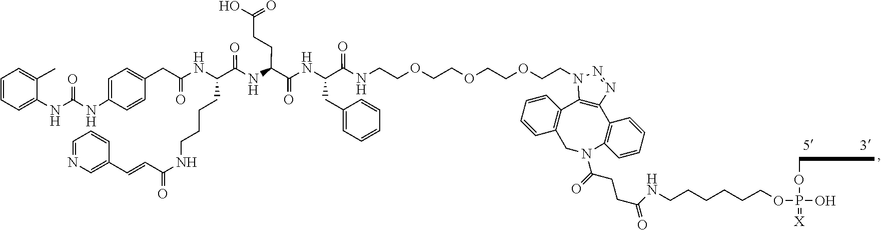

wherein X is S and R is the siRNA targeting Target B (i.e., R is the covalently bound structure of the siRNA).

Compound 10 was evaluated in an in vivo human Target B transgenic mice PD study carried out as described in Example 21, except that instead of intracerebroventricular injection, intracisternal magna injection was performed. Results are presented in table below as percent inhibition of Target B, relative to vehicle control.

TABLE 15
Average Target B Inhibition by Compound 10

| Brain Region | Day 15 |
|---|---|
| Cortex | 19% |
| Hippocampus | 29% |
| Cerebellum | 39% |
| Striatum | 24% |
| Cervical Spinal Cord | 72% |

### INCORPORATION BY REFERENCE

The contents of all references (including literature references, issued patents, published patent applications, and co-pending patent applications) cited throughout this application are hereby expressly incorporated herein in their entireties by reference.

### EQUIVALENTS

Those skilled in the art will recognize, or be able to ascertain using no more than routine experimentation, many equivalents of the specific embodiments described herein. Such equivalents are intended to be encompassed by the following claims.

1. A compound, or a stereoisomer, tautomer, prodrug, or salt thereof, comprising the structure of Formula (I′): 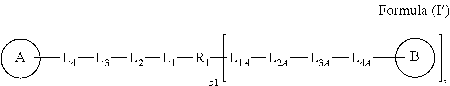
wherein:
each of 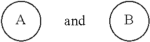
is independently an α₄β_{1/7} integrin ligand;
each of L₁, L₂, L₃, L₄, L_{1A}, L_{2A}, L_{3A}, and L_{4A} is independently a linker, a bond, or absent, wherein at least one of L₁, L₂, L₃, and L₄ is a bond or a linker;
R¹ comprises one or more oligonucleotides, protecting groups, small molecules, proteins, antibodies, and/or peptides; and
z1 is0or 1.

2-5. (canceled)

6. The compound, or a stereoisomer, tautomer, prodrug, or salt thereof, of claim 1, wherein each α₄β_{1/7} integrin ligand is independently selected from the group consisting of: 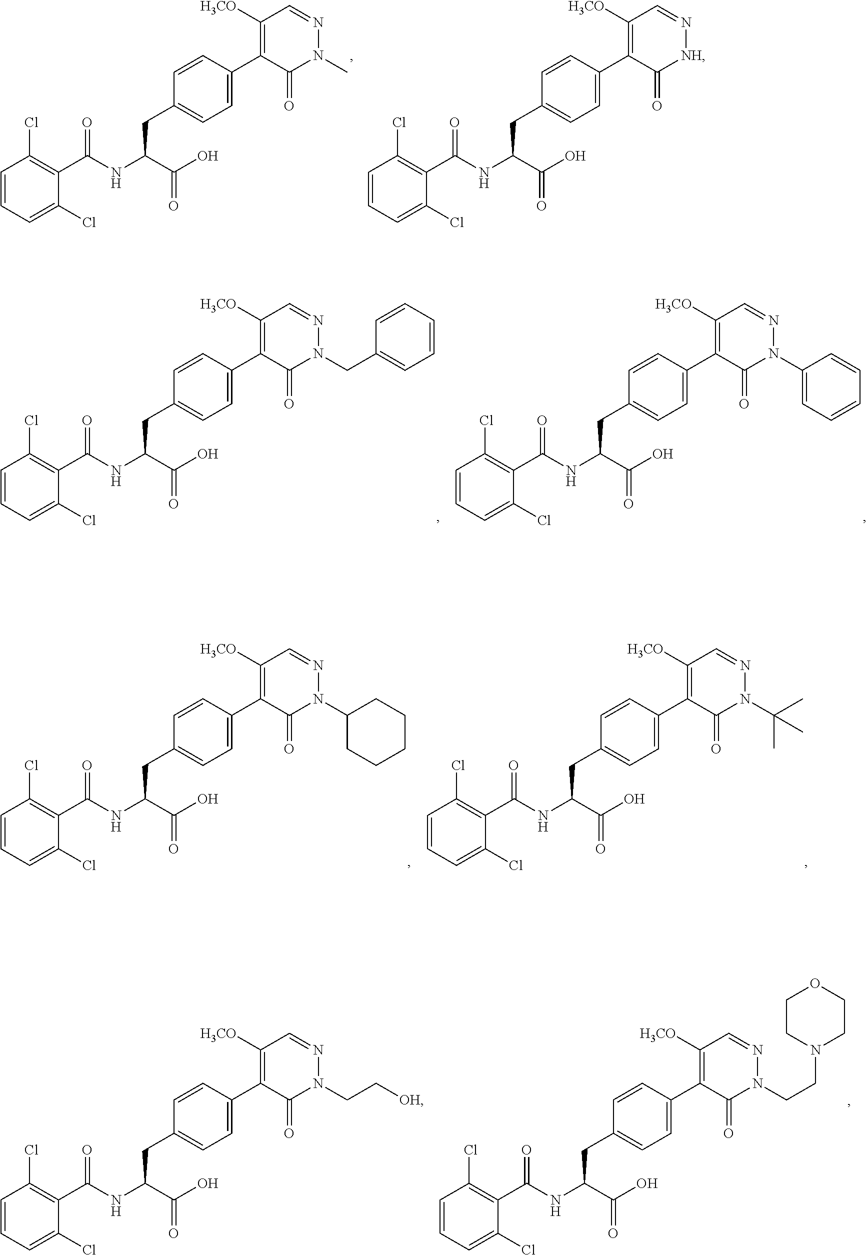 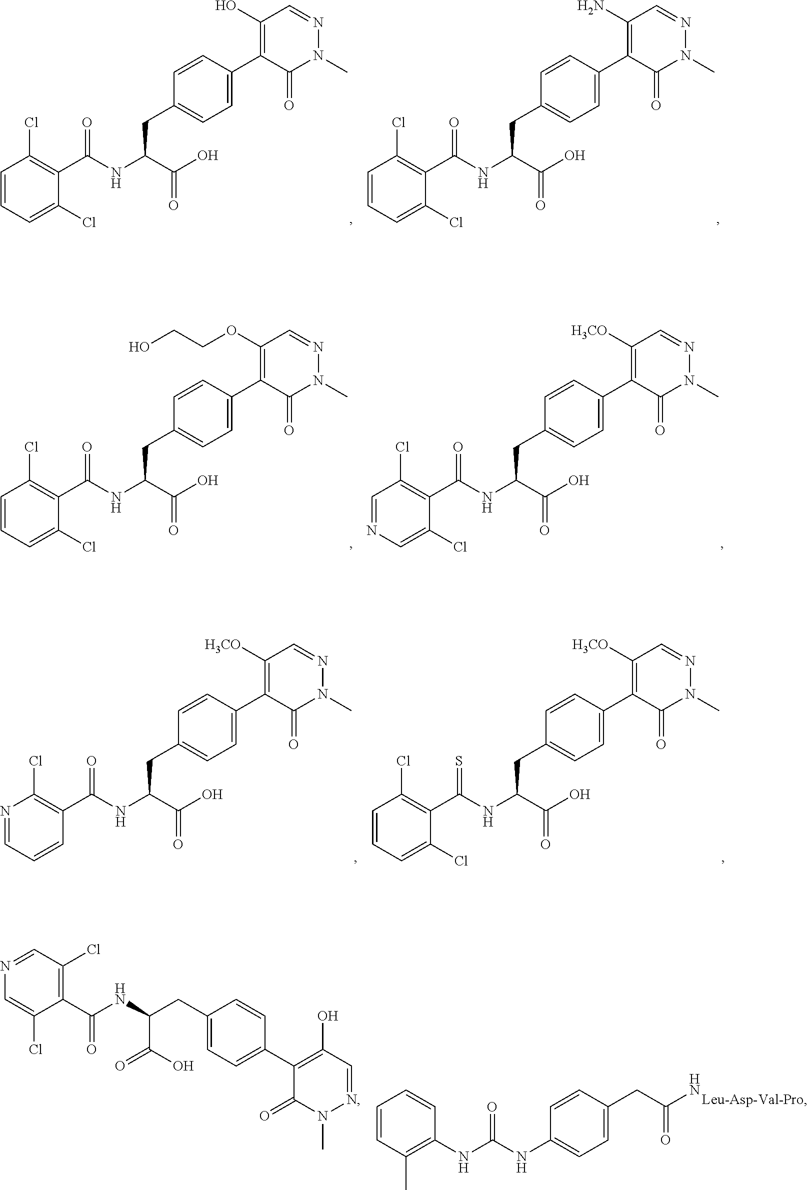 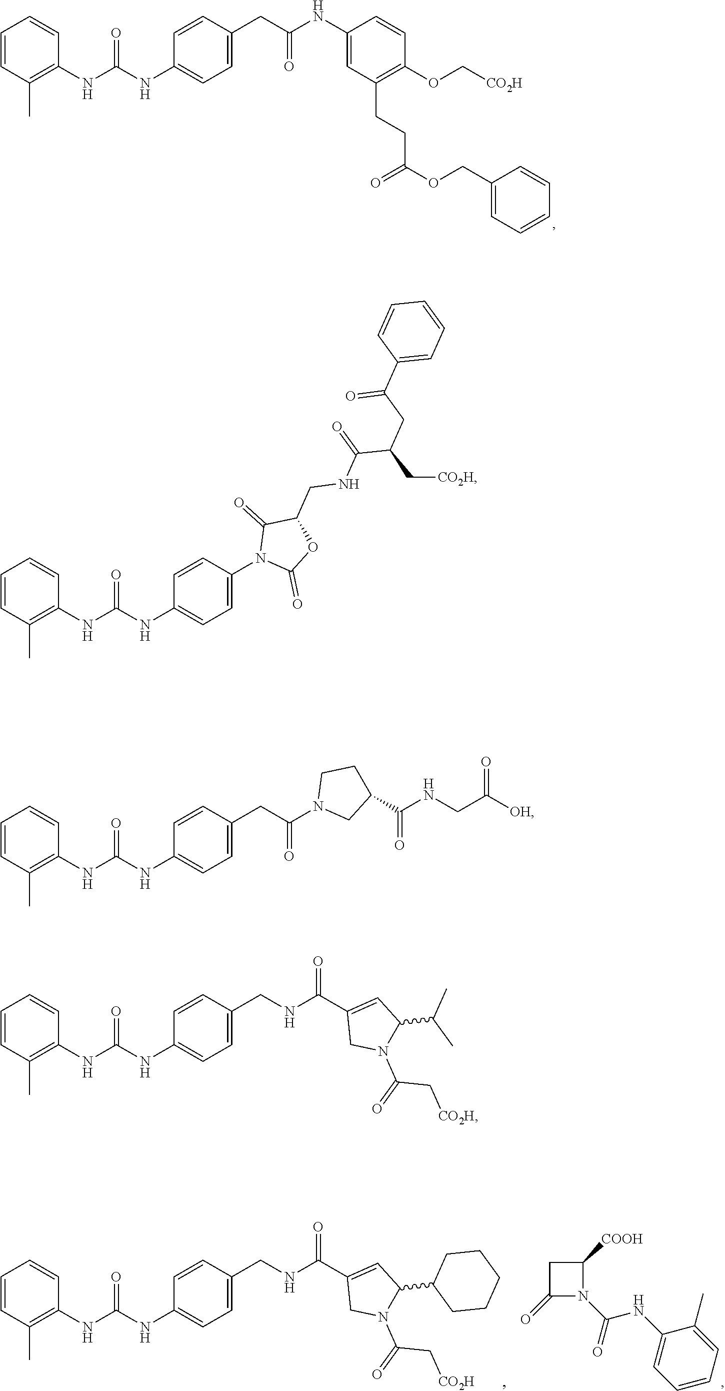 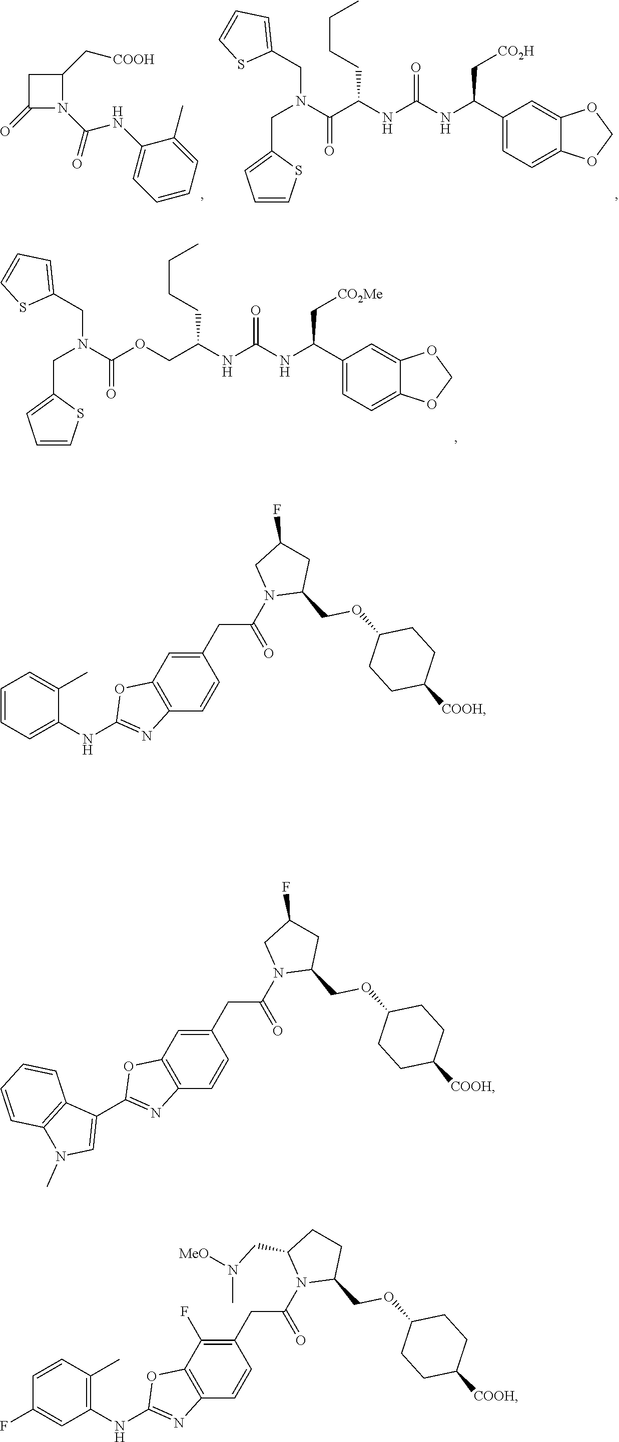 [SMILES: Cc1ccccc1NC(=O)N1C(=O)CC1CC(=O)O ; CCCC[C@H](NC(=O)N[C@@H](CC(=O)O)c1ccc2c(c1)OCO2)C(=O)N(Cc1cccs1)Cc1cccs1 ; CCCC[C@@H](COC(=O)N(Cc1cccs1)Cc1cccs1)NC(=O)N[C@@H](CC(=O)OC)c1ccc2c(c1)OCO2 ; Cc1ccccc1Nc1nc2ccc(CC(=O)N3C[C@@H](F)C[C@H]3CO[C@H]3CC[C@H](C(=O)O)CC3)cc2o1 ; Cn1cc(-c2nc3ccc(CC(=O)N4C[C@@H](F)C[C@H]4CO[C@H]4CC[C@H](C(=O)O)CC4)cc3o2)c2ccccc21 ; CON(C)C[C@@H]1CC[C@@H](CO[C@H]2CC[C@H](C(=O)O)CC2)N1C(=O)Cc1ccc2nc(Nc3cc(F)ccc3C)oc2c1F] 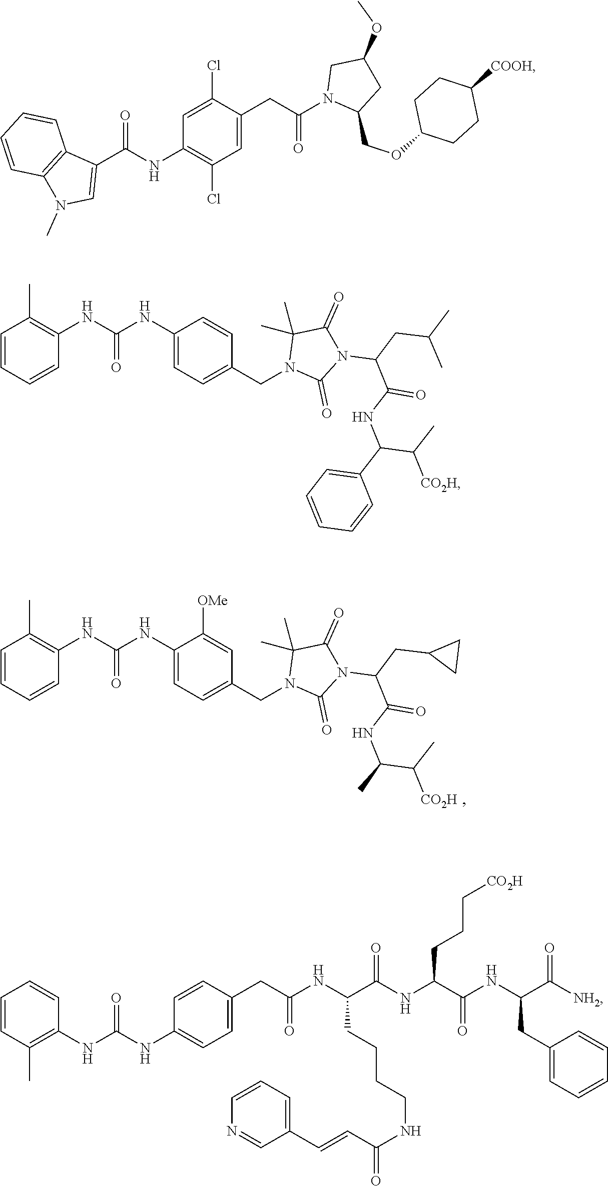 [SMILES: CO[C@H]1C[C@@H](CO[C@H]2CC[C@H](C(=O)O)CC2)N(C(=O)Cc2cc(Cl)c(NC(=O)c3cn(C)c4ccccc34)cc2Cl)C1 ; Cc1ccccc1NC(=O)Nc1ccc(CN2C(=O)N(C(CC(C)C)C(=O)NC(c3ccccc3)C(C)C(=O)O)C(=O)C2(C)C)cc1 ; COc1cc(CN2C(=O)N(C(CC3CC3)C(=O)N[C@H](C)C(C)C(=O)O)C(=O)C2(C)C)ccc1NC(=O)Nc1ccccc1C ; Cc1ccccc1NC(=O)Nc1ccc(CC(=O)N[C@@H](CCCCNC(=O)/C=C/c2cccnc2)C(=O)N[C@@H](CCCC(=O)O)C(=O)N[C@H](Cc2ccccc2)C(N)=O)cc1] 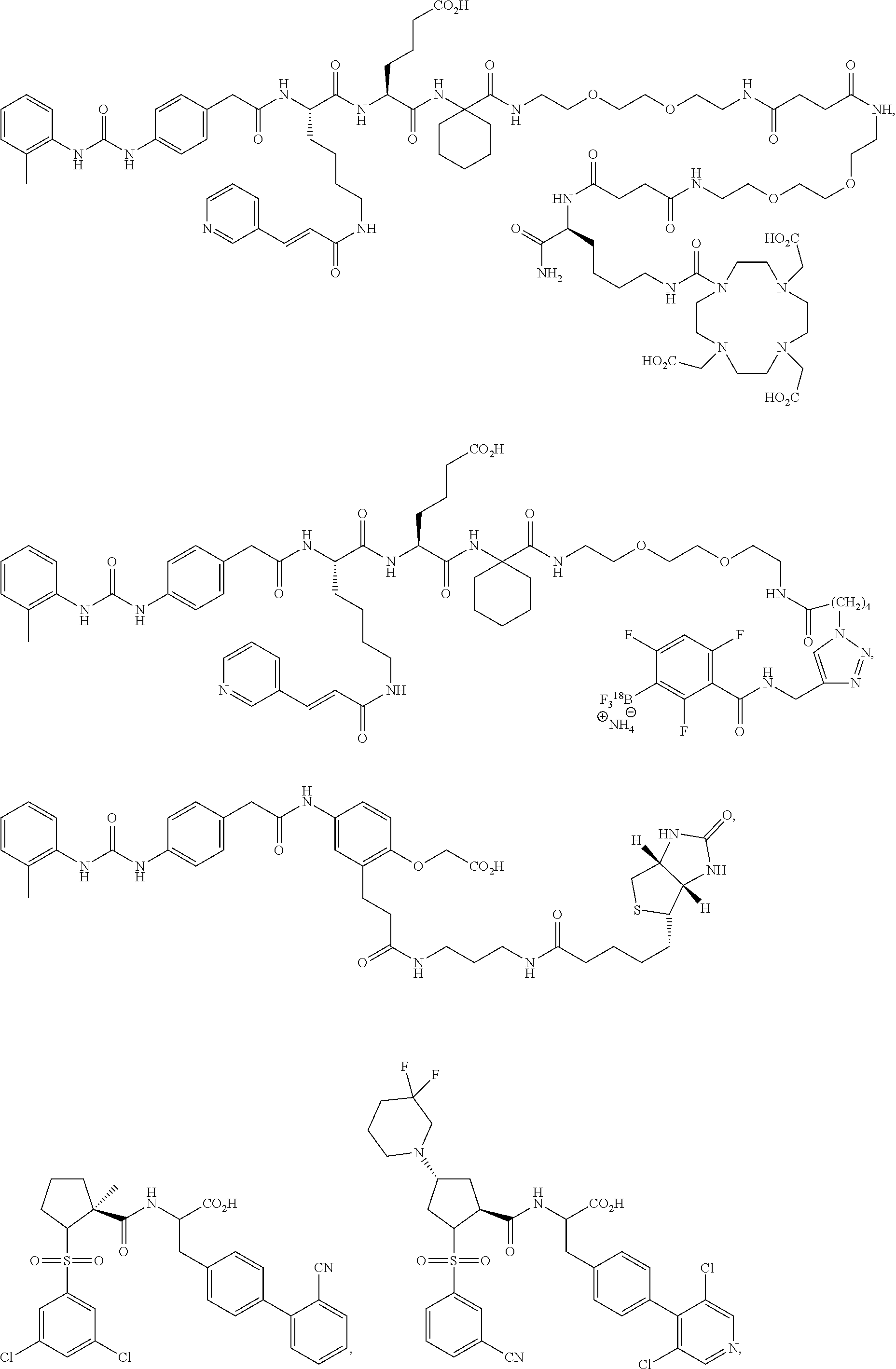 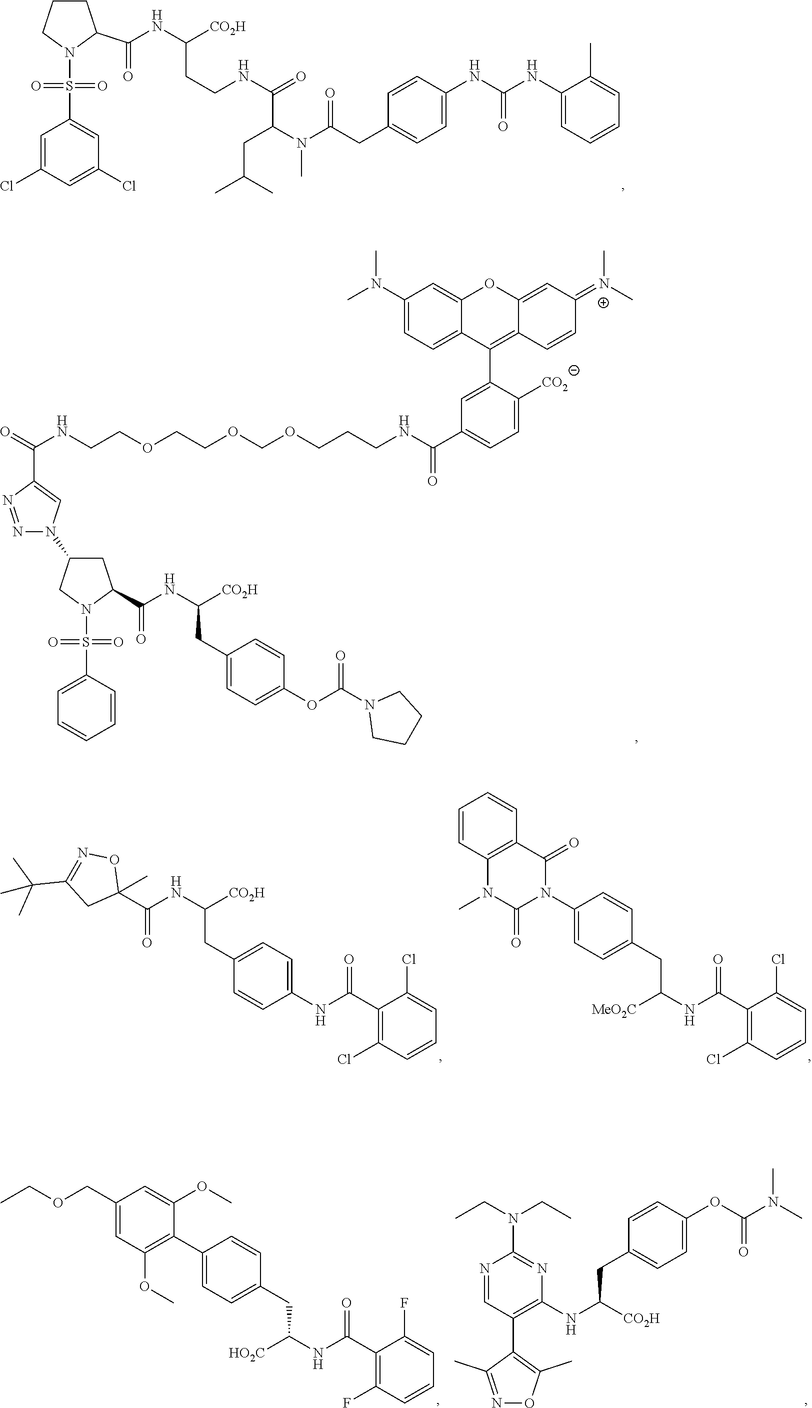 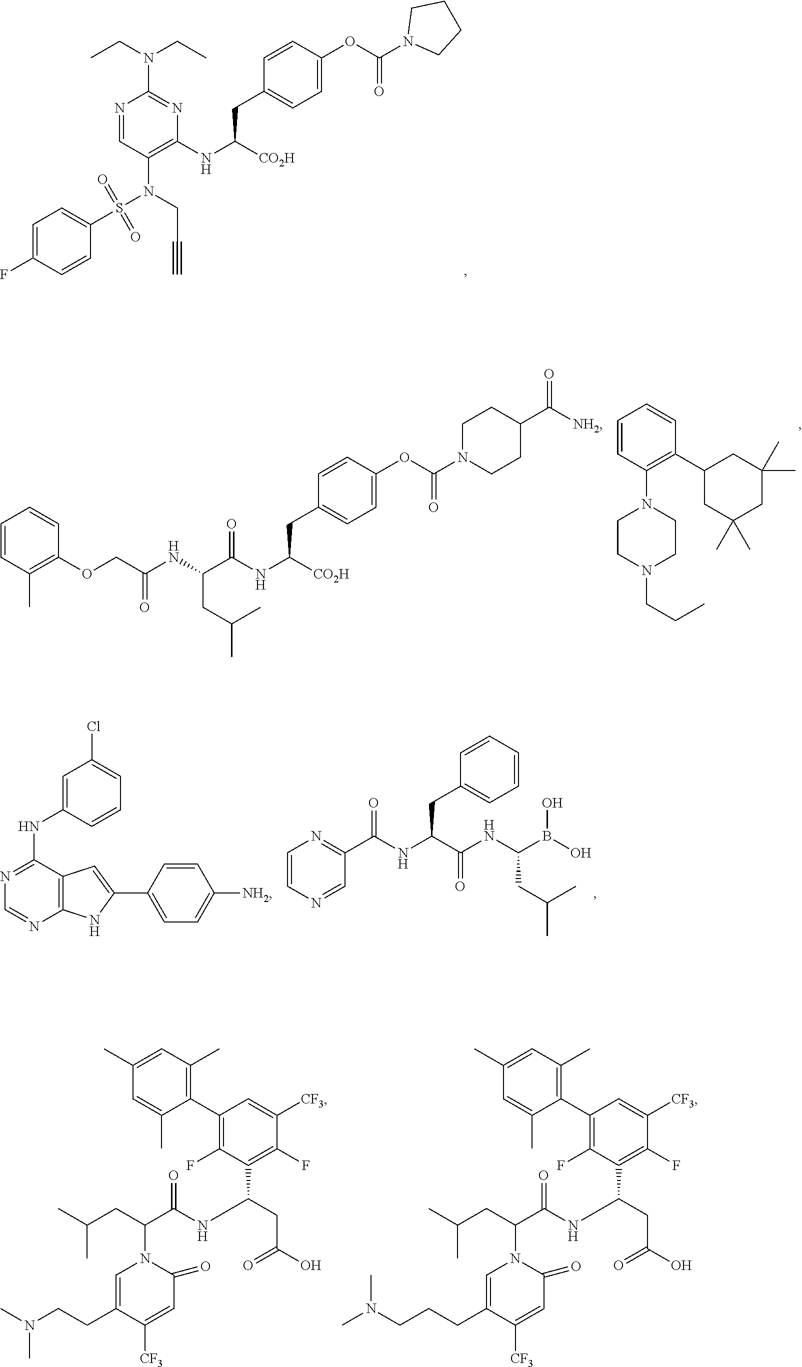 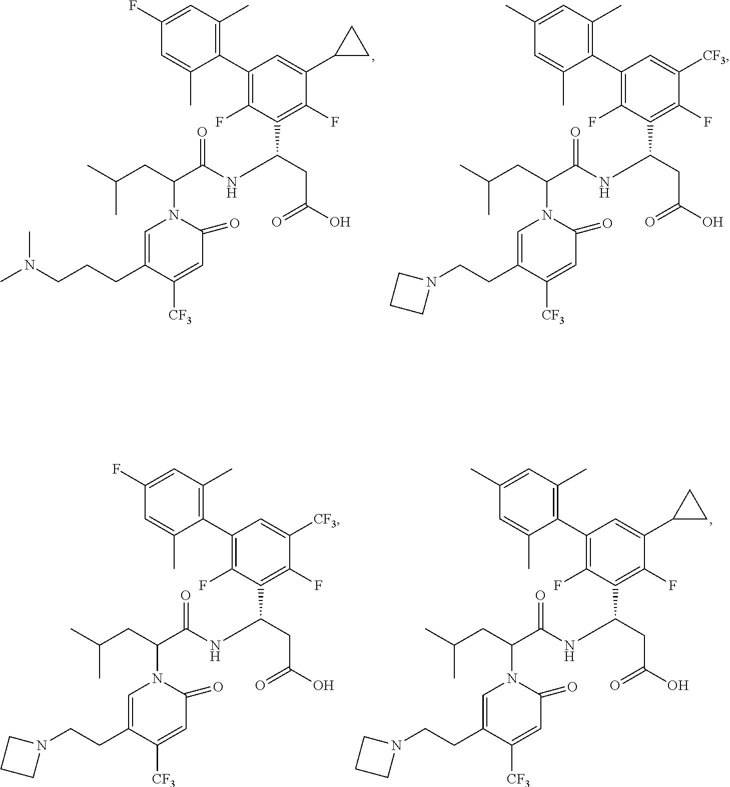 [SMILES: Cc1cc(F)cc(C)c1-c1cc(C2CC2)c(F)c([C@H](CC(=O)O)NC(=O)C(CC(C)C)n2cc(CCCN(C)C)c(C(F)(F)F)cc2=O)c1F ; Cc1cc(C)c(-c2cc(C(F)(F)F)c(F)c([C@H](CC(=O)O)NC(=O)C(CC(C)C)n3cc(CCN4CCC4)c(C(F)(F)F)cc3=O)c2F)c(C)c1 ; Cc1cc(F)cc(C)c1-c1cc(C(F)(F)F)c(F)c([C@H](CC(=O)O)NC(=O)C(CC(C)C)n2cc(CCN3CCC3)c(C(F)(F)F)cc2=O)c1F ; Cc1cc(C)c(-c2cc(C3CC3)c(F)c([C@H](CC(=O)O)NC(=O)C(CC(C)C)n3cc(CCN4CCC4)c(C(F)(F)F)cc3=O)c2F)c(C)c1] 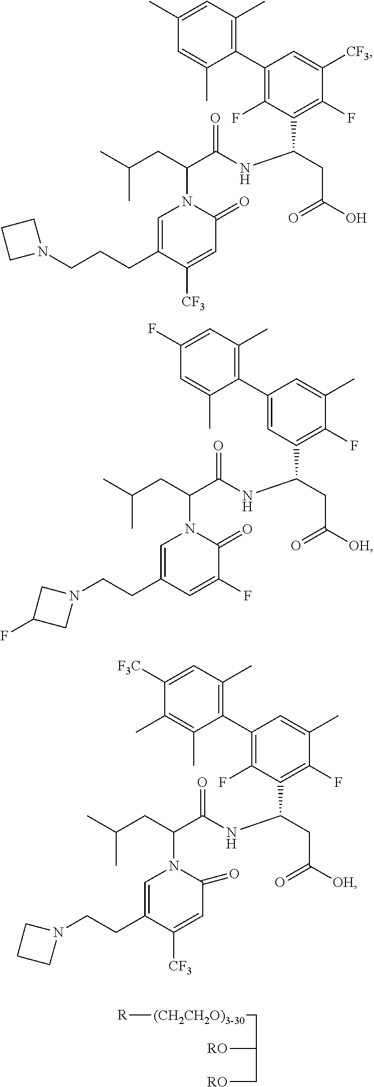
wherein each instance of R is 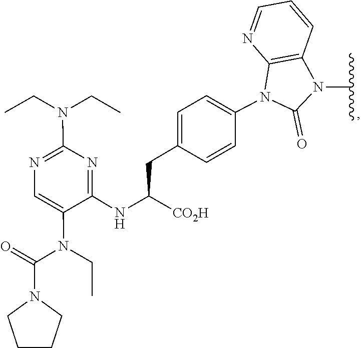 [SMILES: *n1c(=O)n(-c2ccc(C[C@H](Nc3nc(N(CC)CC)ncc3N(CC)C(=O)N3CCCC3)C(=O)O)cc2)c2ncccc21]
an anti-α₄β_{1/7} integrin antibody, and derivatives thereof.

7. The compound, or a stereoisomer, tautomer, prodrug, or salt thereof, of claim 1, wherein the compound comprises the structure of Formula (II′): 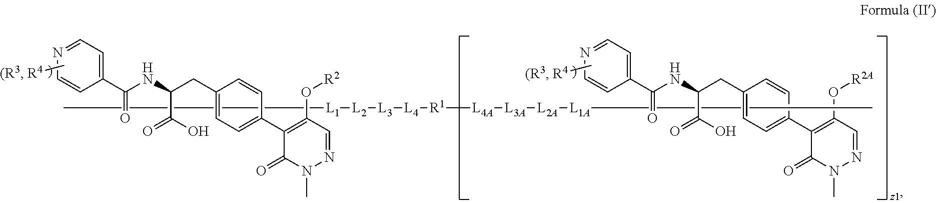
wherein:
R² and R^{2A} are each independently H, polyethylene glycol (PEG), optionally substituted heteroalkyl, or optionally substituted heteroaryl; and
R³, R^{3A}, R⁴, and R^{4A} are each independently H, halogen, optionally substituted alkyl, or optionally substituted —O-alkyl.

8-14. (canceled)

15. The compound, or a stereoisomer, tautomer, prodrug, or salt thereof, of claim 1, wherein the compound comprises the structure of Formula (III′): 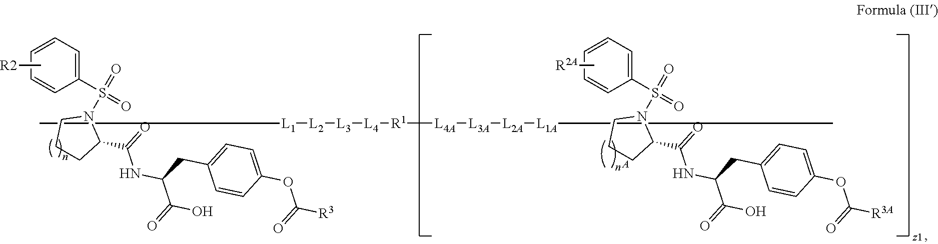
wherein:
R² and R^{2A} are each independently H, halogen, polyethylene glycol (PEG), optionally substituted alkyl, optionally substituted heteroalkyl, optionally substituted heteroaryl, optionally substituted —O-alkyl, or optionally substituted cycloalkyl;
R³ and R^{3A} are each independently optionally substituted heteroalkyl or optionally substituted heterocyclyl; and
n and n^{A} are each independently 1, 2, or 3.

16-18. (canceled)

19. The compound, or a stereoisomer, tautomer, prodrug, or salt thereof, of claim 1, wherein the compound comprises the structure of Formula (IV′): 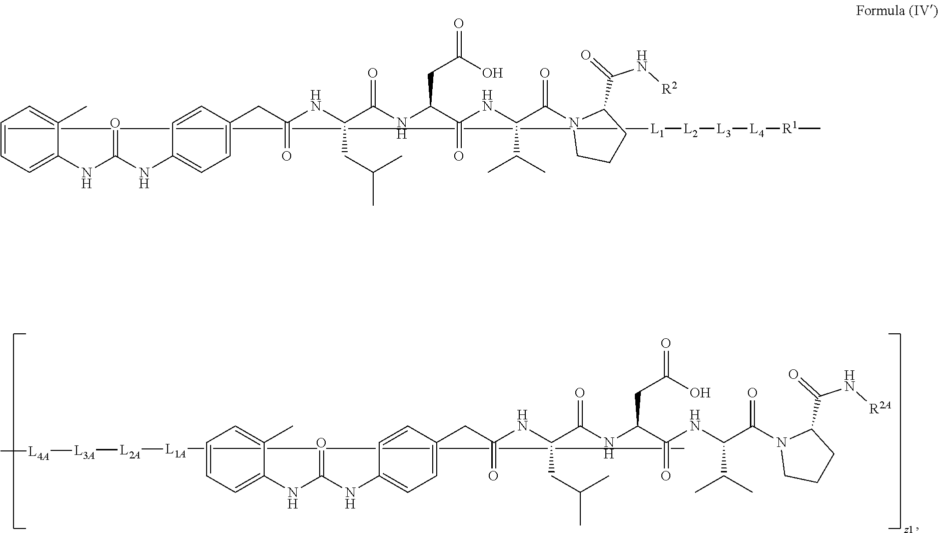
wherein:
R² and R^{2A} are each independently H, —OH, —NH₂, —NHR³, —OR³, or absent; and
each instance of R³ is independently H, polyethylene glycol, optionally substituted alkyl, optionally substituted heteroalkyl, optionally substituted cycloalkyl, or optionally substituted heteroaryl.

20-23. (canceled)

24. The compound, or a stereoisomer, tautomer, prodrug, or salt thereof, of claim 1, wherein the compound comprises the structure of Formula (V′): 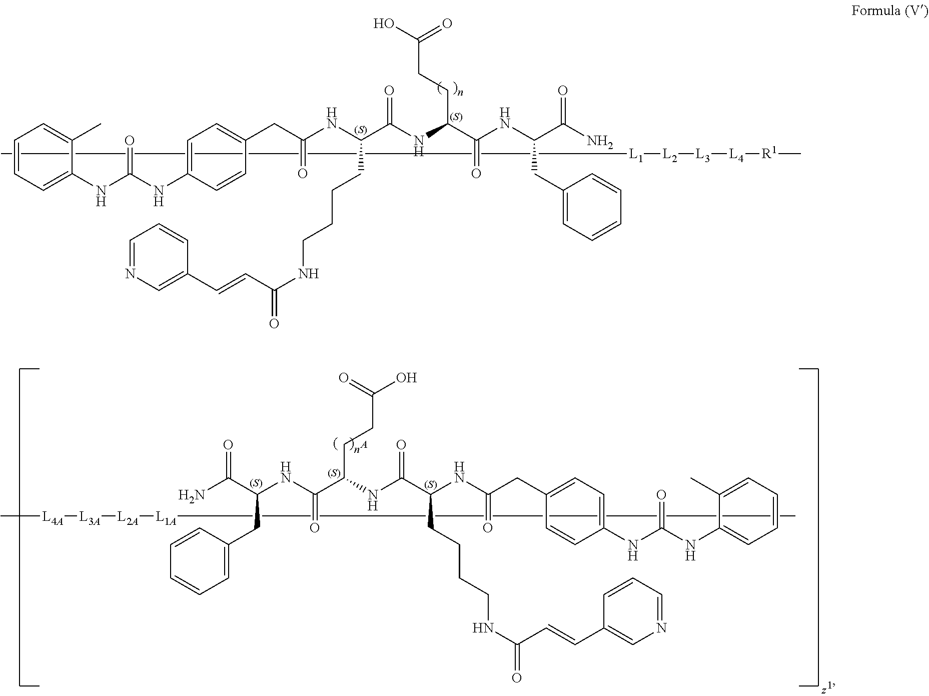
wherein:
n and n^{A} are each independently 0, 1, 2, or3.

25-31. (canceled)

32. The compound, or a stereoisomer, tautomer, prodrug, or salt thereof, of claim 1, wherein the compound comprises the structure of Formula (VI′): 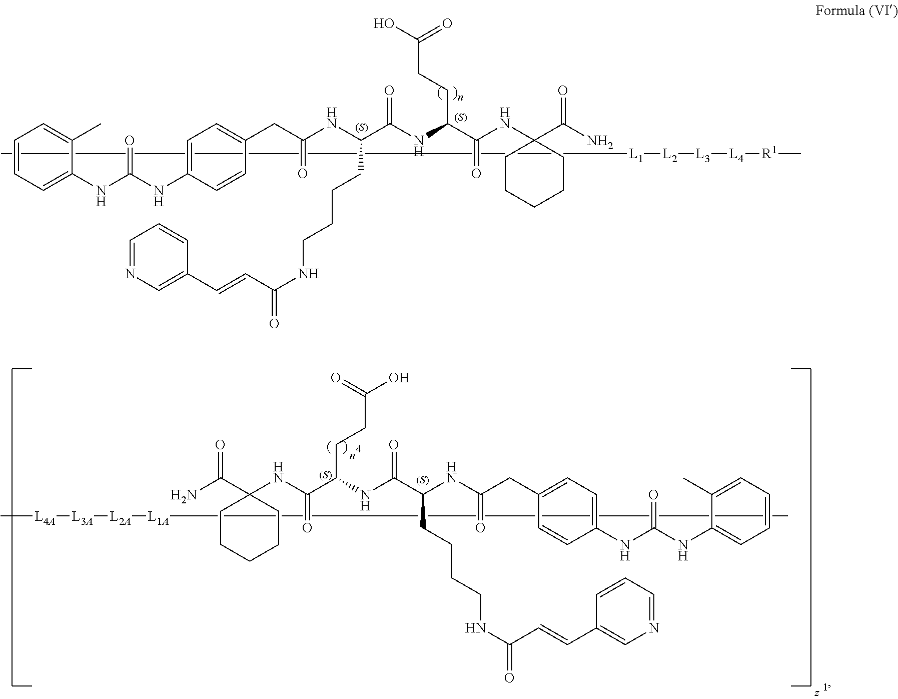
wherein:
n and n^{A} are each independently 0, 1, 2, or 3.

33-38. (canceled)

39. The compound, or a stereoisomer, tautomer, prodrug, or salt thereof, of claim 1, wherein the compound comprises the structure of Formula (VII′): 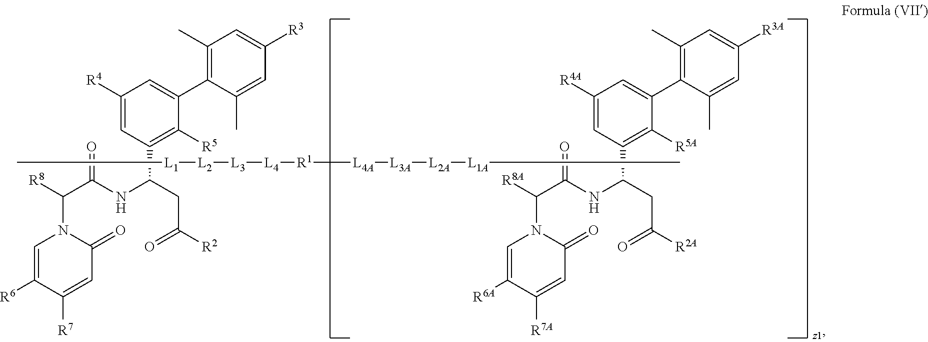
wherein:
R², R^{2A}, R³, R^{3A}, R⁴, R^{4A}, R⁵, and R^{5A} are each independently H, halogen, optionally substituted alkyl, optionally substituted —O-alkyl, cycloalkyl, or absent;
R⁸ and R^{8A} are each independently optionally substituted C₁-C₅ alkyl, optionally substituted C₁-C₅ alkylene-(C₃-C₆)-cycloalkyl, or optionally substituted (C₁-C₄)-alkylene-(C₁-C₄)-alkoxy; and
R⁶, R^{R6A}, R⁷, and R^{7A} are each independently H, halogen, alkyl, optionally substituted alkyl, optionally substituted heteroalkyl,

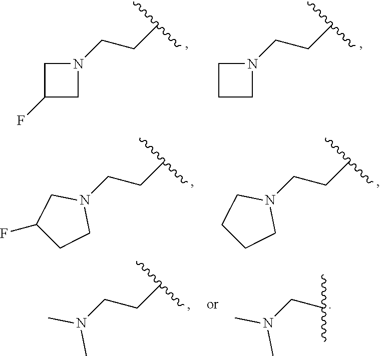

40-61. (canceled)

62. The compound, or a stereoisomer, tautomer, prodrug, or salt thereof, of claim 1, wherein the compound comprises the structure of Formula (VIII′): 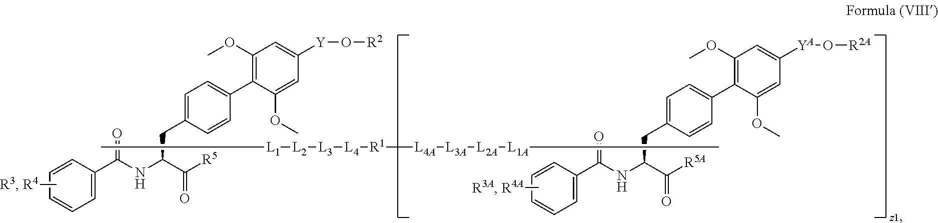
wherein:
R² and R^{2A} are each independently H, polyethylene glycol, optionally substituted alkyl, optionally substituted heteroalkyl, optionally substituted cycloalkyl, optionally substituted heteroaryl, or absent;
R³, R^{3A}, R⁴, and R^{4A}are each independently H, halogen, optionally substituted alkyl, or optionally substituted —O-alkyl;
R⁵ and R^{5A} are each independently —OH or absent; and
Y and Y^{A} are each independently —CH₂— or —(CH₂)₂—.

63-68. (canceled)

69. The compound, or a stereoisomer, tautomer, prodrug, or salt thereof, of claim 1, wherein the compound comprises the structure of Formula (IX′): 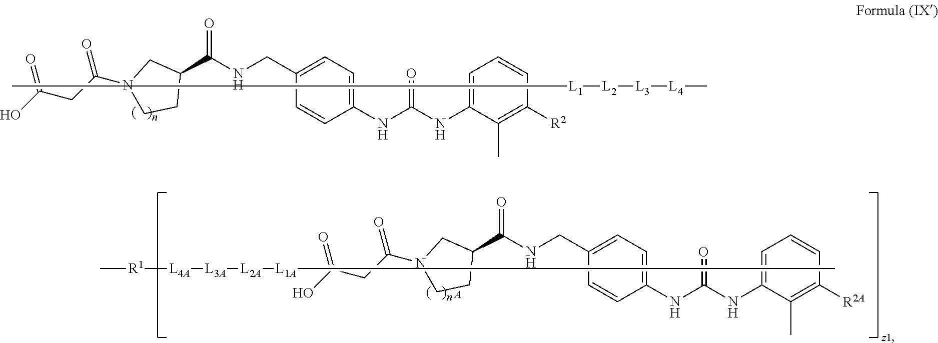
wherein:
each of R² and R^{2A} is independently H, —OH, —NH₂, —NHR³, —OR³, or —CONHR³;
each instance of R³ is independently H, polyethylene glycol, optionally substituted alkyl, optionally substituted heteroalkyl, optionally substituted cycloalkyl, or optionally substituted heteroaryl; and
each of n and n^{A} is independently 1 or 2.

70-72. (canceled)

73. The compound, or a stereoisomer, tautomer, prodrug, or salt thereof, of claim 1, wherein the compound comprises the structure of Formula (X′): 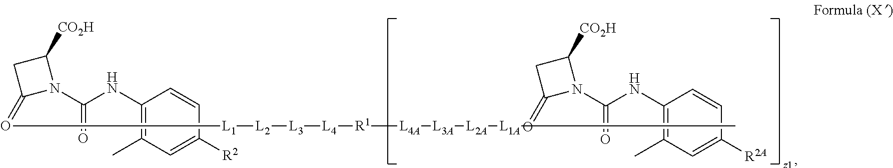
wherein:
R² and R^{2A} are each independently H, —CH₂OR³, —(CH₂)₂OR³, —CH₂NHCOR³, or —OR³; and
each instance of R³ is independently H, polyethylene glycol, optionally substituted alkyl, optionally substituted heteroalkyl, optionally substituted cycloalkyl, or optionally substituted heteroaryl.

74-76. (canceled)

77. The compound, or a stereoisomer, tautomer, prodrug, or salt thereof, of claim 1, wherein the compound comprises the structure of Formula (XI′): 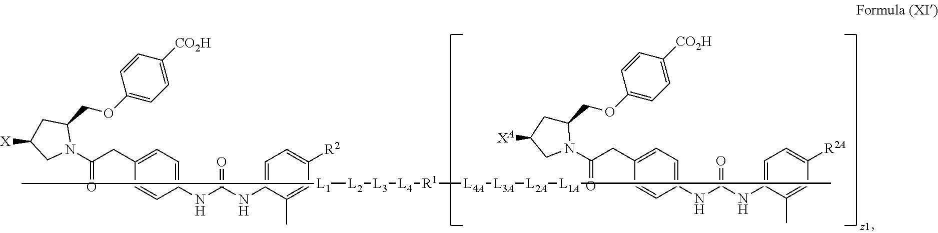
wherein:
each of R² and R^{2A} is independently H, —CONHR³, —CH₂OR³, —(CH₂)₂OR³, —CH₂NHCOR³, or —OR³;
each instance of R³ is independently H, polyethylene glycol, optionally substituted alkyl, optionally substituted heteroalkyl, optionally substituted cycloalkyl, or optionally substituted heteroaryl; and
each of X and X^{A} are independently H or halogen.

78-80. (canceled)

81. The compound, or a stereoisomer, tautomer, prodrug, or salt thereof, of claim 1, wherein the compound comprises the structure of Formula (XII′): 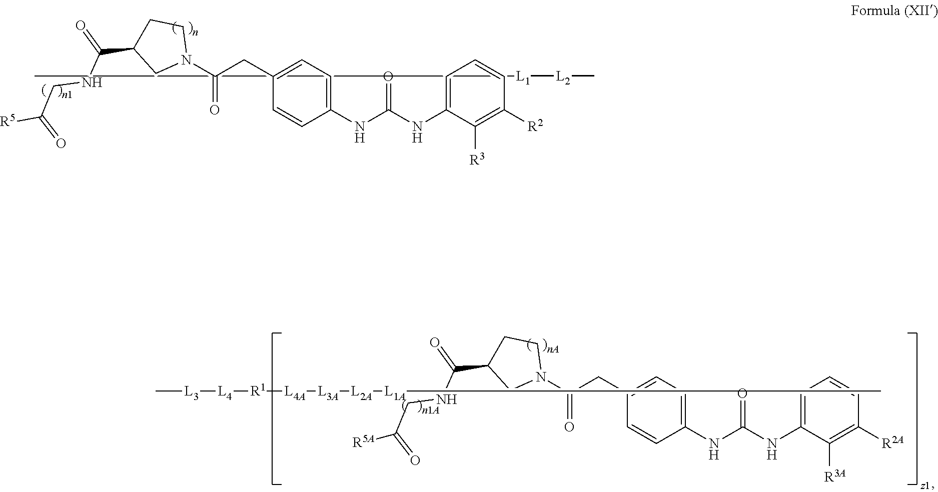
wherein:
each of R² and R^{2A} is independently H, —CONHR⁴, —CH₂OR⁴, —(CH₂)₂OR⁴, —CH₂NHCOR⁴, or —OR⁴;
each of R³ and R^{3A} is independently H, optionally substituted alkyl, or optionally substituted cycloalkyl;
each instance of R⁴ is independently H, polyethylene glycol, optionally substituted alkyl, optionally substituted heteroalkyl, optionally substituted cycloalkyl, or optionally substituted heteroaryl;
each of R⁵ and R^{5A} is independently —OH or absent;
each instance of n and n^{A} is independently 0, 1, 2, or 3; and
each instance of n1 and n1^{A} is independently 1, 2, or 3.

82-84. (canceled)

85. The compound, or a stereoisomer, tautomer, prodrug, or salt thereof, of claim 1, wherein the compound comprises the structure of Formula (XIII′): 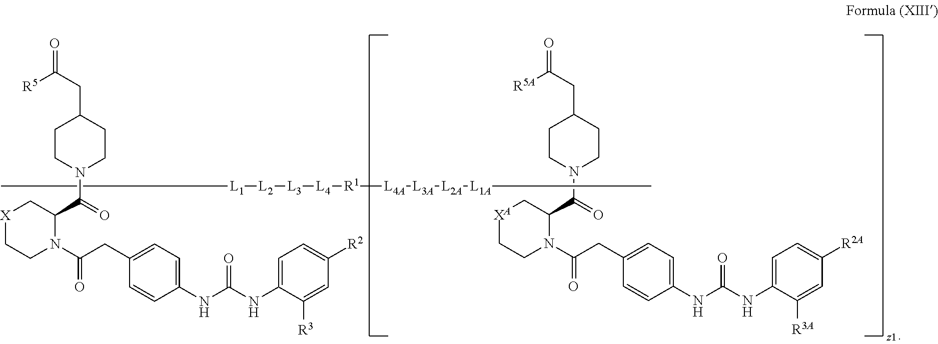
wherein:
each of R² and R^{2A} is independently H, —CONHR⁴, —CH₂OR⁴, —(CH₂)₂OR⁴, —CH₂NHCOR⁴, or —OR⁴;
each of R³ and R^{3A} is independently H, optionally substituted alkyl, or optionally substituted cycloalkyl;
each instance of R⁴ is independently H, polyethylene glycol, optionally substituted alkyl, optionally substituted heteroalkyl, optionally substituted cycloalkyl, or optionally substituted heteroaryl;
each of R⁵ and R^{5A} is independently —OH or absent; and
each of X and X^{A} is independently H, optionally substituted CH₂, optionally substituted NH, or cycloalkyl.

86-89. (canceled)

90. The compound, or a stereoisomer, tautomer, prodrug, or salt thereof, of claim 1, wherein the compound comprises the structure of Formula (XIV′): 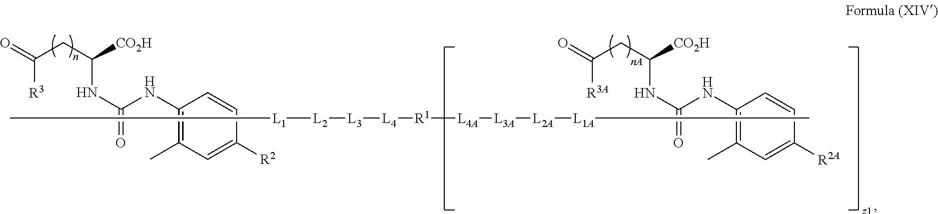
wherein:
each of R² and R^{2A} is independently H, —CH₂OR⁴, —(CH₂)₂OR⁴, —CH₂NHCOR⁴, or —OR⁴;
each of R³ and R^{3A} is independently H, —OH, —NH₂, —NHR⁵, or —OR⁵;
each instance of R⁴ is independently H, polyethylene glycol, optionally substituted alkyl, optionally substituted heteroalkyl, optionally substituted cycloalkyl, or optionally substituted heteroaryl;
each instance of R⁵ is independently H, polyethylene glycol, optionally substituted alkyl, optionally substituted heteroalkyl, optionally substituted cycloalkyl, or optionally substituted heteroaryl; and
each of n and n^{A} is independently 1, 2, or 3.

91-107. (canceled)

108. The compound, or a stereoisomer, tautomer, prodrug, or salt thereof, of claim 1, wherein the compound comprises the structure: 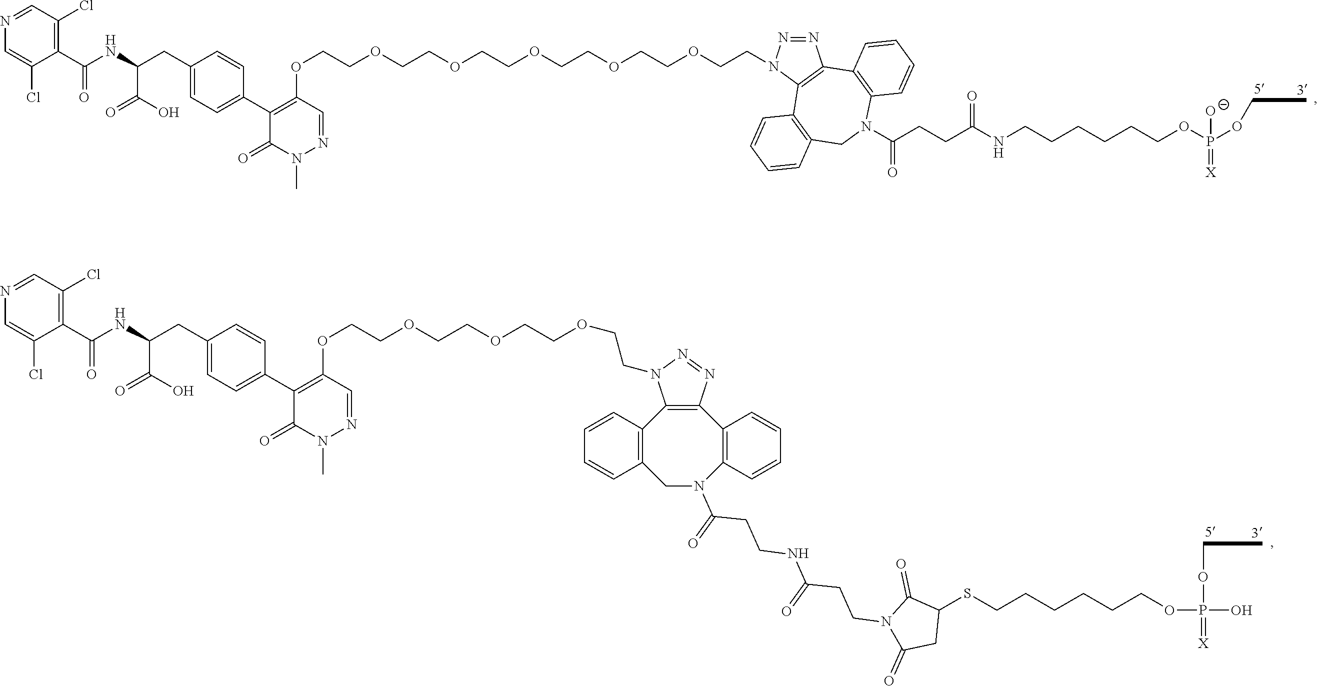 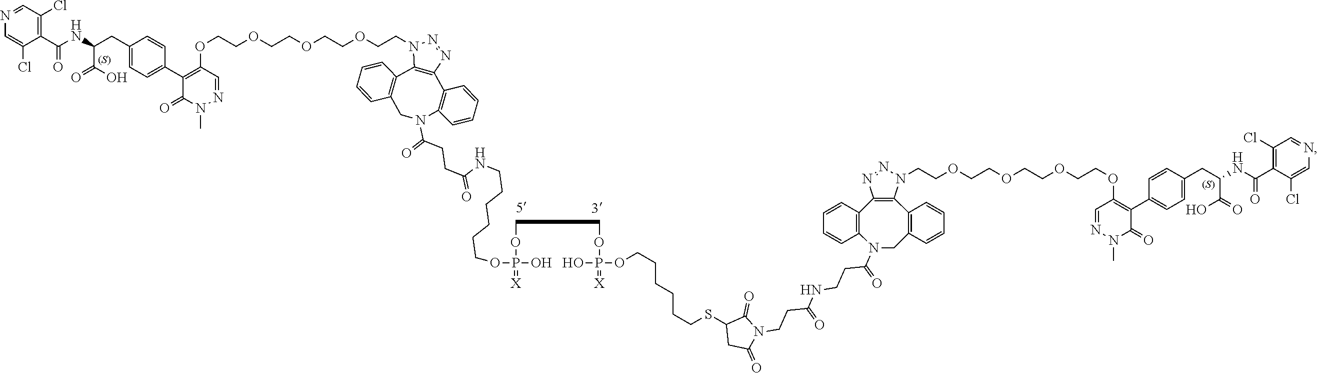 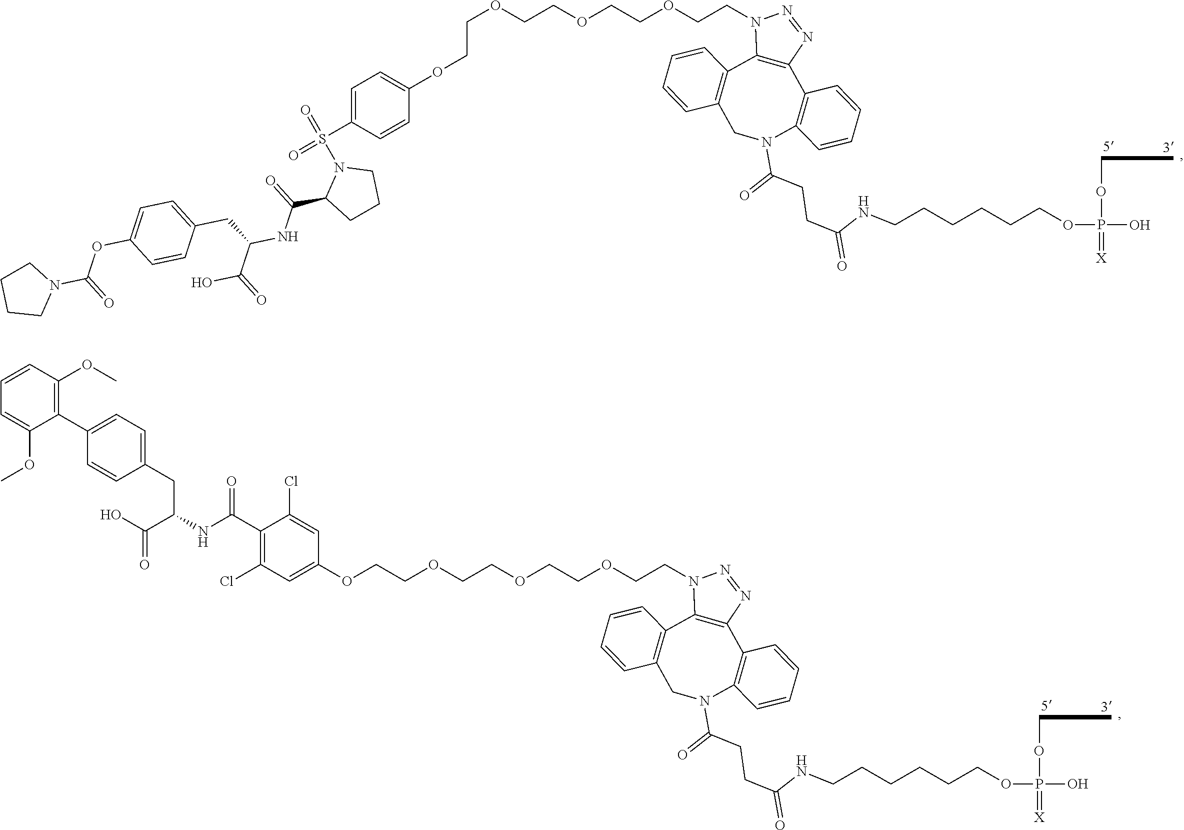 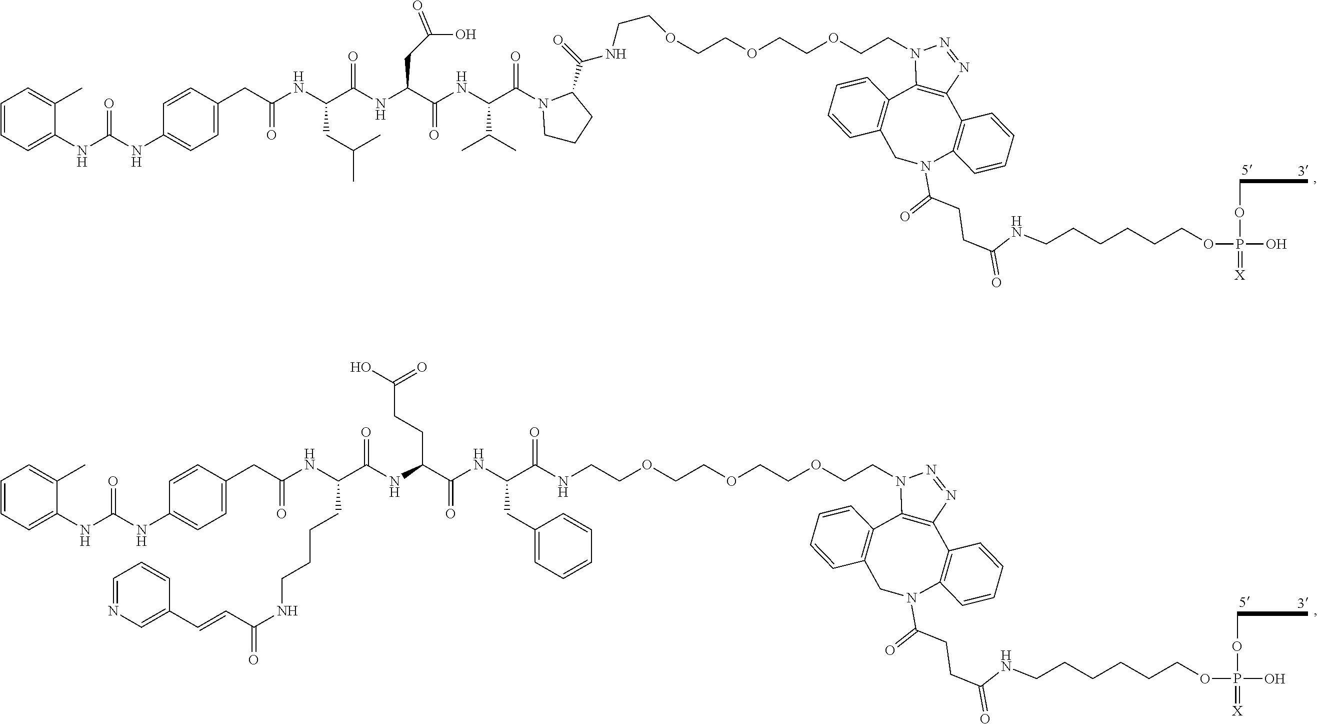 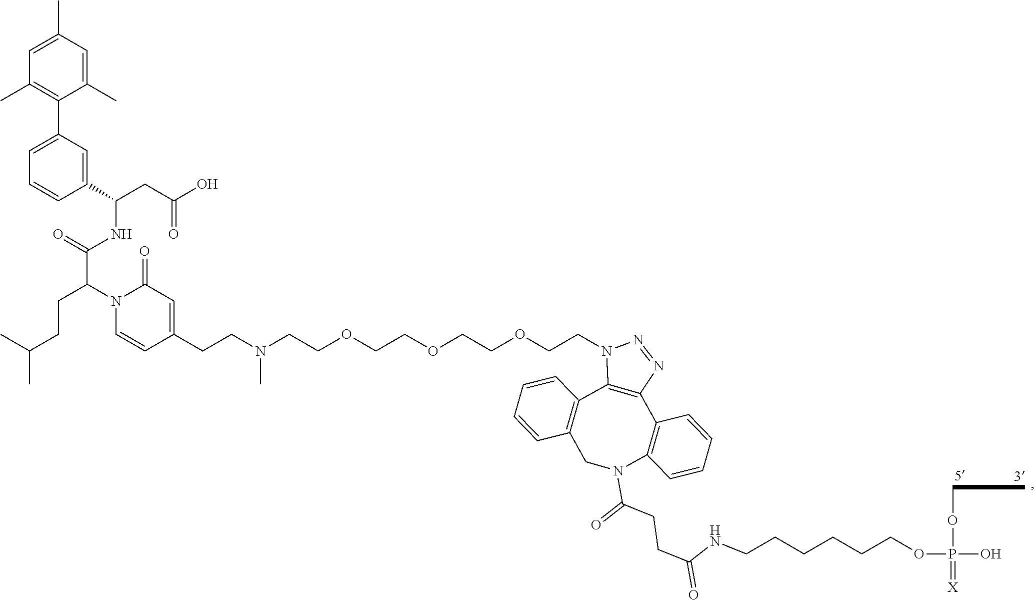 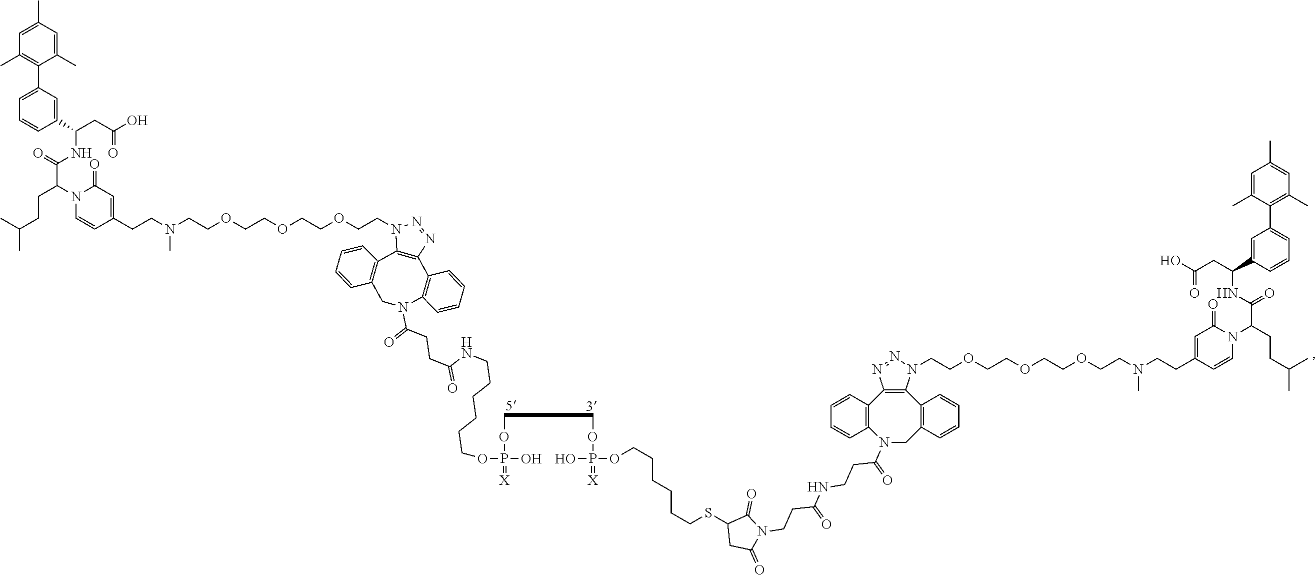 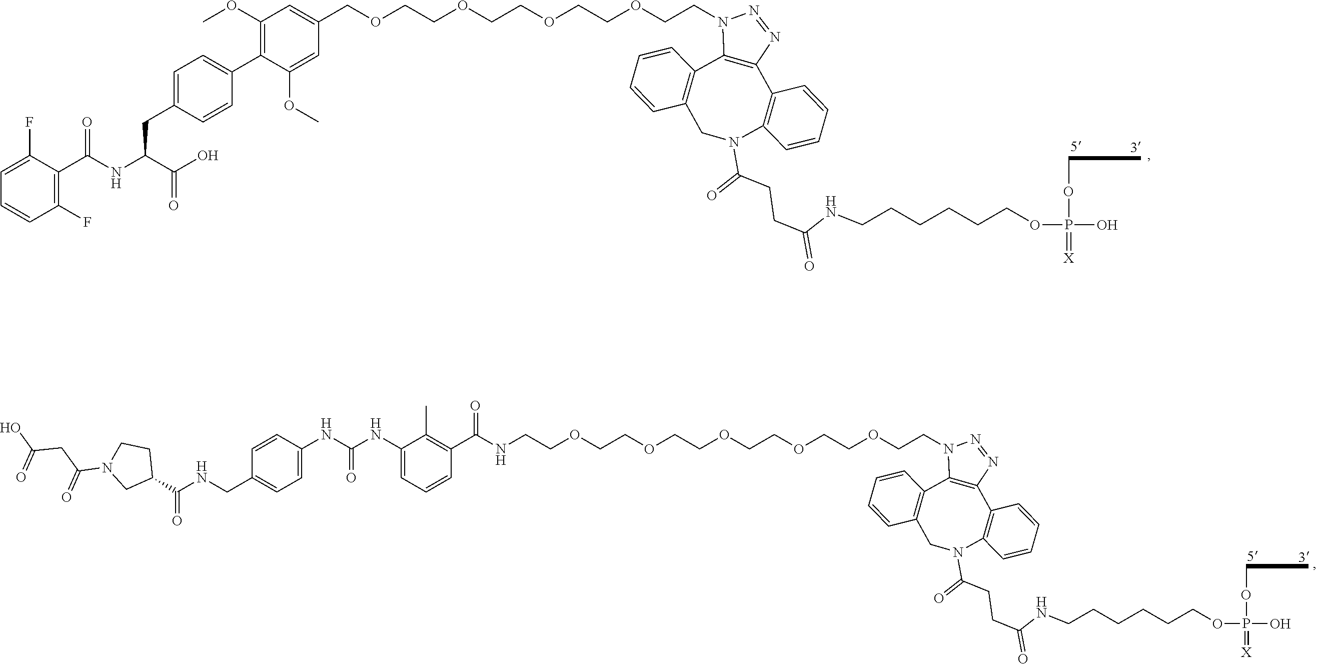 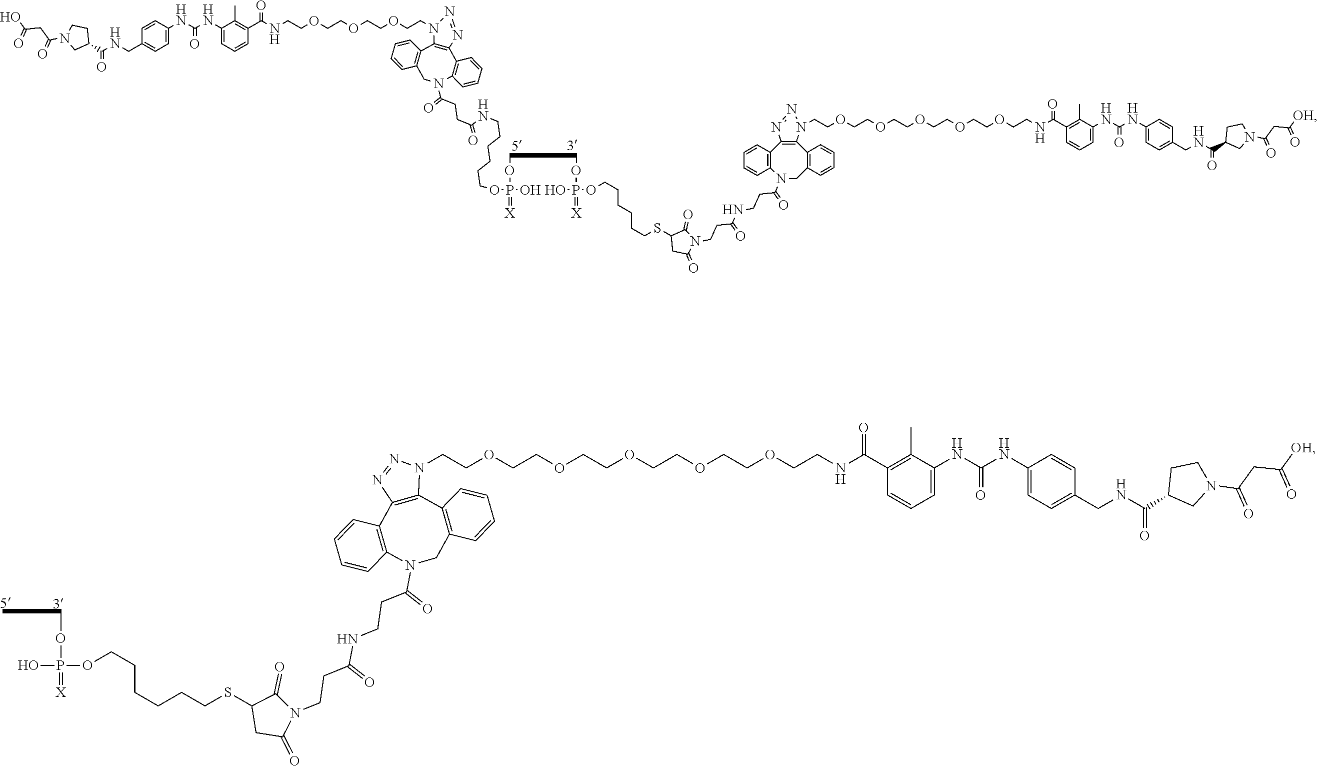 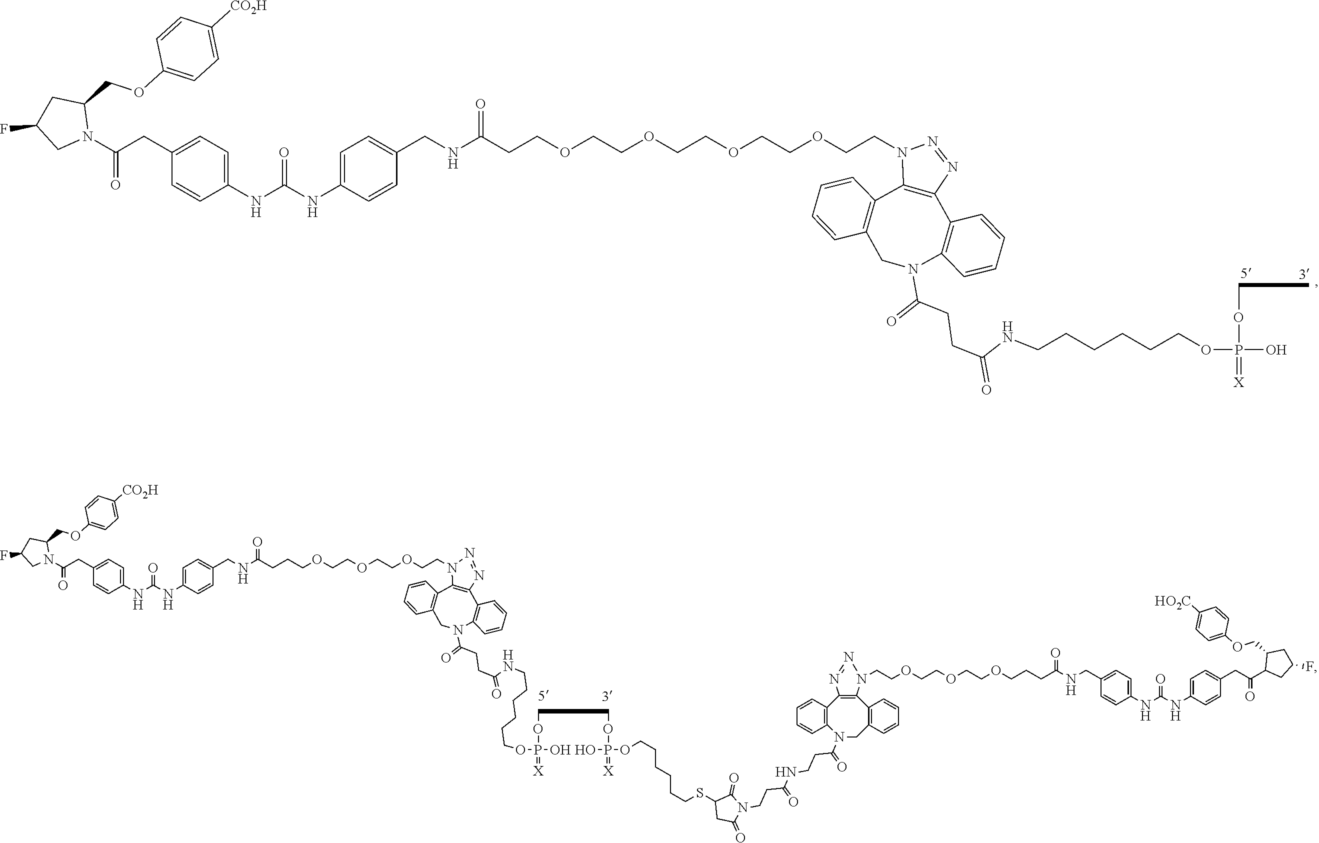 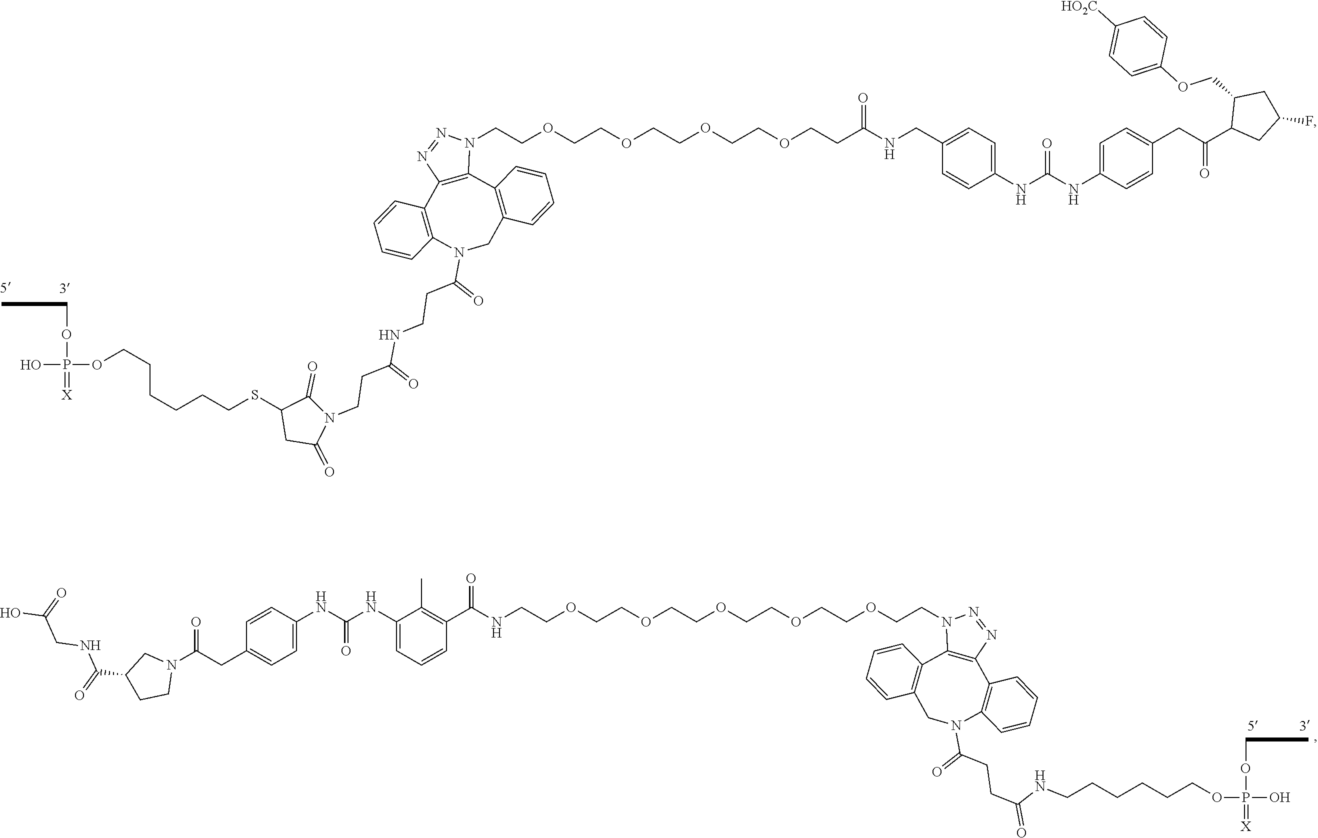 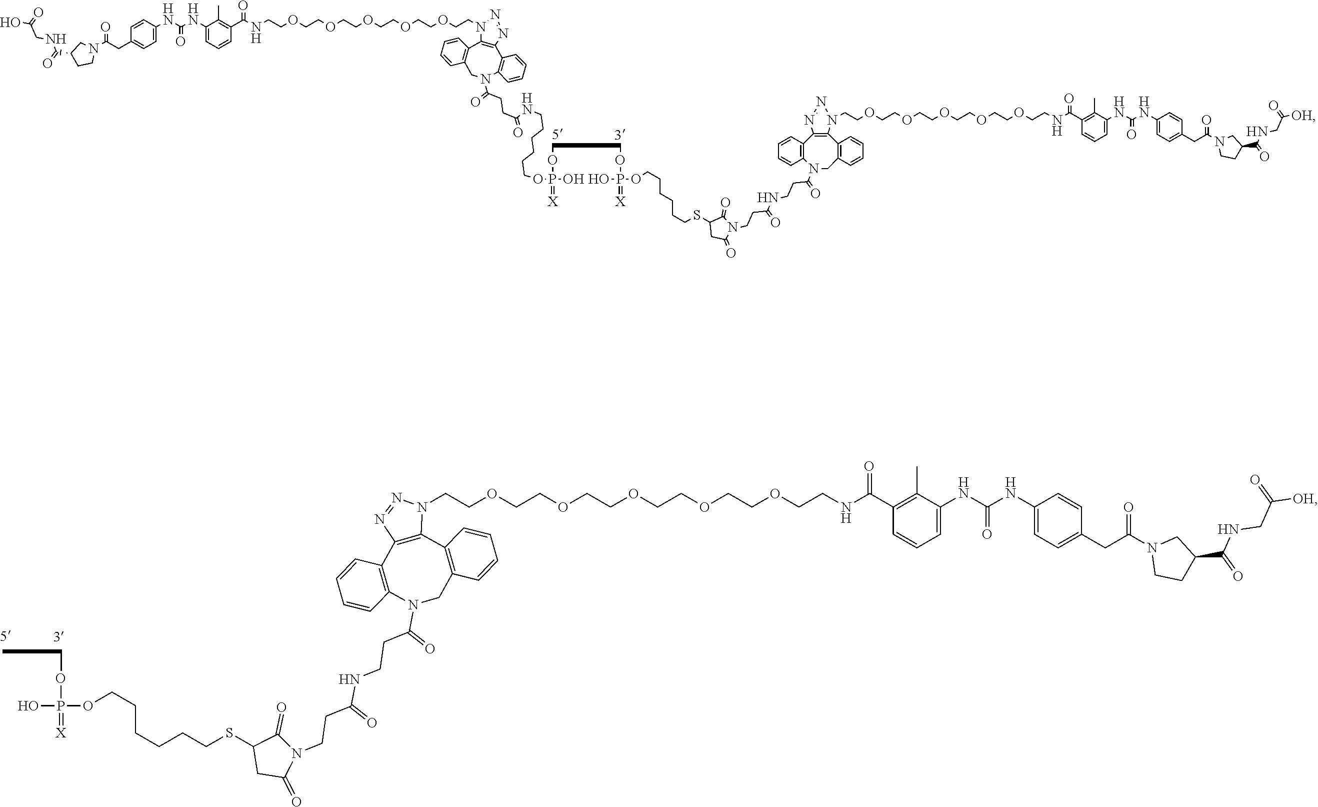 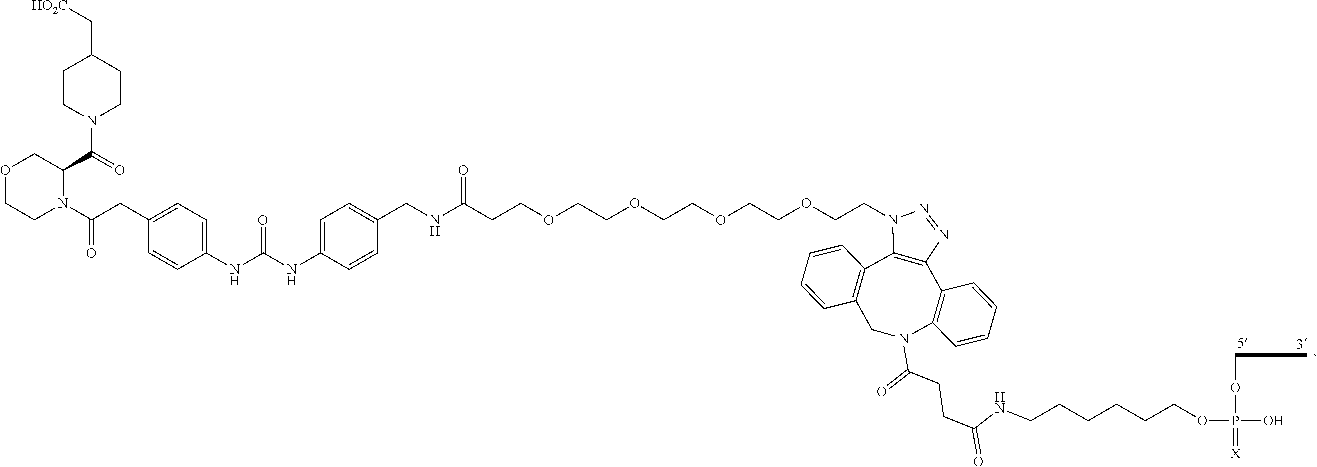 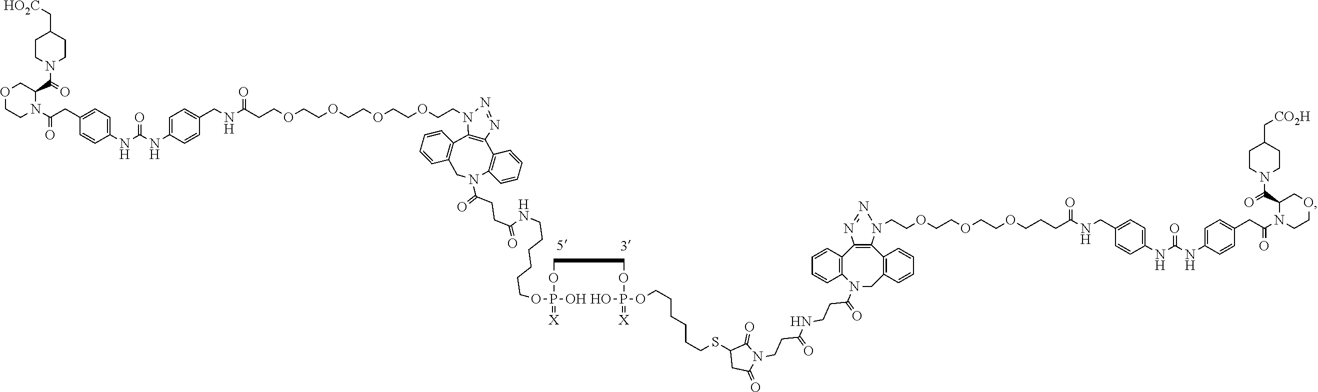 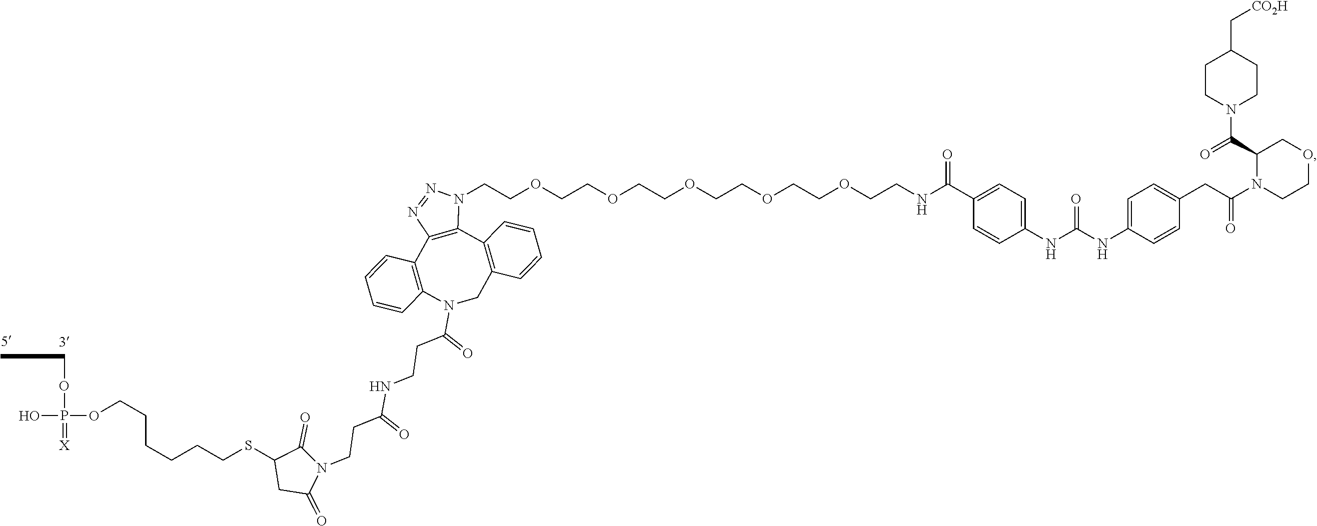 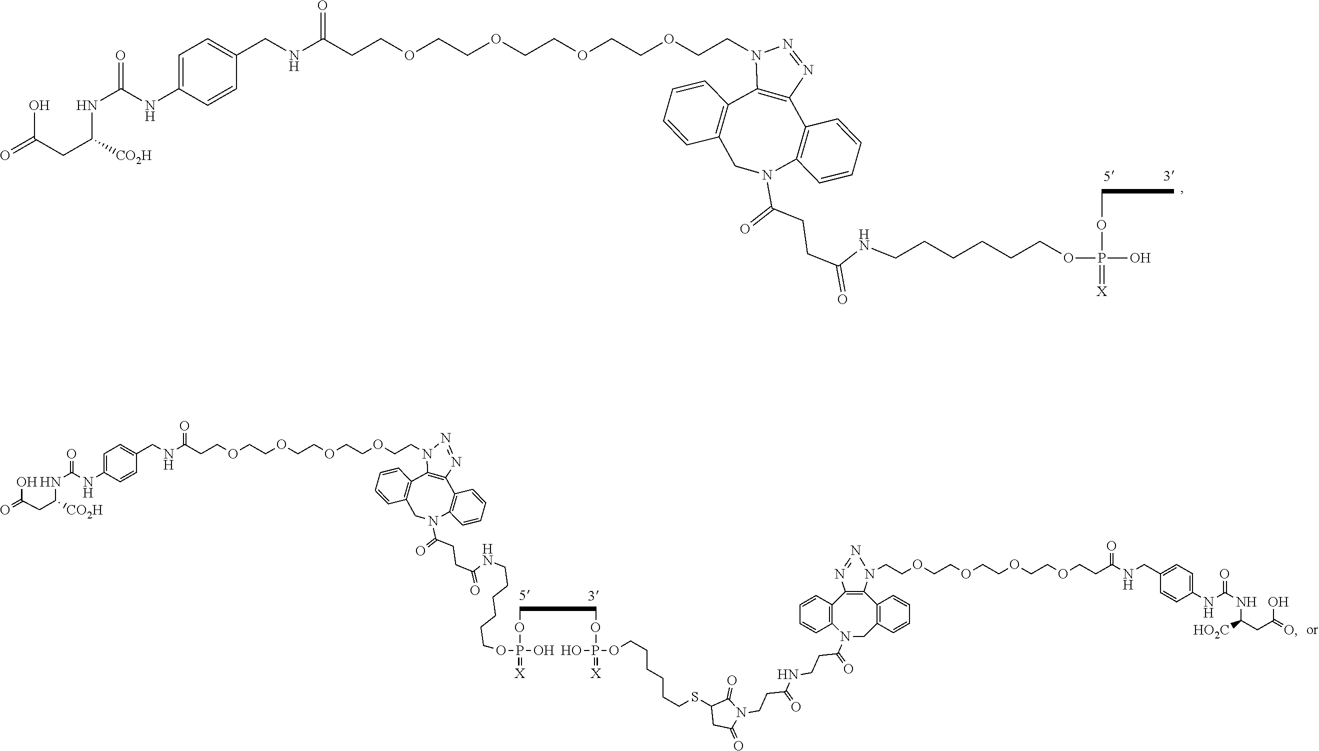 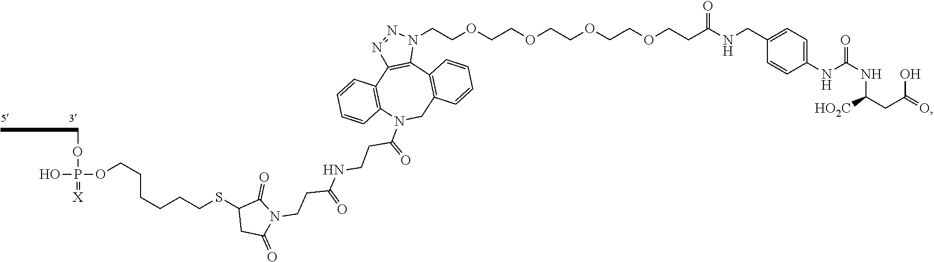
wherein:
R¹ comprises an oligonucleotide; [symbol]
is the oligonucleotide; and
X is 0 or S.

109-110. (canceled)

111. The compound, or a stereoisomer, tautomer, prodrug, or salt thereof, of claim 1, wherein R¹ comprises a modified oligonucleotide.

112-119. (canceled)

120. A composition comprising a compound, or a stereoisomer, tautomer, prodrug, or salt thereof, of claim 1, and a pharmaceutically acceptable excipient.

121-122. (canceled)

123. A method for treating or ameliorating a central nervous system (CNS) disease, disorder, or symptom thereof in a subject, comprising administration of the compound, or a stereoisomer, tautomer, prodrug, or salt thereof, of claim 1, to the subject.

124. (canceled)

125. The method of claim 123, wherein the CNS disease, disorder, or symptom thereof is Alzheimer's disease, or a symptom thereof.

126-127. (canceled)
